(19) United States
(12) Patent Application Publication
(10) Pub. No.: US 2023/0227829 A1
(43) Pub. Date: Jul. 20, 2023

(54) METHODS AND COMPOSITIONS FOR TREATING EPILEPSY

(71) Applicants: INSERM (INSTITUT NATIONAL DE LA SANTÉ ET DE LA RECHERCHE MÉDICALE), Paris (FR); UNIVERSITÉ D'AIX-MARSEILLE, Marseille (FR); UNIVERSITÉ DE BORDEAUX, Bordeaux (FR); REGENXBIO INC., Rockville, MD (US); CENTRE NATIONAL DE LA RECHERCHE SCIENTIFIQUE, Paris (FR); CORLIEVE THERAPEUTICS, Illkirch-Graffenstaden (FR)

(72) Inventors: Valérie CREPEL, Marseille (FR); Christophe MULLE, Pessac (FR); Céline BOILEAU, Marseille (FR); Séverine DEFORGES, Bruges (FR); Olivier DANOS, Rockville, MD (US); Andrew MERCER, Rockville, MD (US); Richard PORTER, Binningen (CH); April R. TEPE, Rockville, MD (US)

(21) Appl. No.: 18/154,241

(22) Filed: Jan. 13, 2023

Related U.S. Application Data
(60) Provisional application No. 63/299,758, filed on Jan. 14, 2022.

(51) Int. Cl. C12N 15/113; C12N 15/86; A61P 25/08

(52) U.S. Cl. CPC C12N 15/113; C12N 15/86; A61P 25/08; C12N 2310/11; C12N 2750/14143

(57) ABSTRACT

The present disclosure provides methods and compositions relating to gene therapy for treating epilepsy, such as a temporal lobe epilepsy, in a subject in need thereof by targeting Grik2 mRNA. In particular, the present disclosure provides inhibitory RNA constructs capable of inhibiting expression of GluK2 protein and inhibiting epileptiform discharges in hyperexcitable neuronal circuit.

### SEQUENCE LISTING

The instant application contains a Sequence Listing which has been submitted electronically in XML format and is hereby incorporated by reference in its entirety. Said XML copy, created on Jan. 12, 2023, is named “51460-010002_Sequence_Listing_1_12 _23” and is 892,424 bytes in size.

### FIELD OF THE DISCLOSURE

The disclosure is in the field of epilepsy. In particular, the disclosure relates to methods and compositions for treating an epilepsy, such as, e.g., temporal lobe epilepsy.

### BACKGROUND

Temporal lobe epilepsy (TLE) is the most common form of partial epilepsy in adults (30-40% of all forms of epilepsies). It is well established that the hippocampus plays a key role in the pathophysiology of TLE. In human patients and animal models of TLE, an aberrant rewiring of neuronal circuits occurs. One of the best examples of network reorganization (“reactive plasticity”) is the sprouting of recurrent mossy fibers (rMF) that establish novel pathophysiological glutamatergic synapses onto dentate granule cells (DGCs) in the hippocampus (Tauck and Nadler, 1985; Represa et al., 1989a, 1989b; Sutula et al., 1989; Gabriel et al., 2004) leading to a recurrent excitatory loop. rMF synapses operate through ectopic kainate receptors (KARs) (Epsztein et al., 2005; Artinian et al., 2011, 2015). KARs are tetrameric glutamate receptors assembled from GluK1-GluKsubunits. In heterologous expression systems, GluK1, GluK2, and GluK3 may form homomeric receptors, while GluK4 and GluK form heteromeric receptors in conjunction with GluK1-3 subunits. Native KARs are widely distributed in the brain with high densities of receptors found in the hippocampus (Carta et al, 2016, EJN), a key structure involved in TLE. Prior studies by the present inventors have established that epileptic activities including interictal spikes and ictal discharges were markedly reduced in mice lacking the GluK2 KAR subunit. Moreover, epileptiform activities were strongly reduced following the use of pharmacological small molecule antagonists of GluK2/GluK5-containing KARs, which block ectopic synaptic KARs (Peret et al., 2014). These data show that KARs ectopically expressed at rMFs in DGCs play a major role in chronic seizures in TLE. Therefore, aberrant KARs expressed in DGCs and composed of GluK2/GluK5 are considered to represent a promising target for the treatment of pharmaco-resistant TLE.

RNA interference (RNAi) strategies have been proposed for many disease targets. Successful application of RNAi-based therapies has been limited. RNAi therapeutics face multiple challenges such as prediction of susceptible off-target domains to inform RNA design, variable in vivo gene silencing efficacies, and reduction of off-target effects, especially where complex gene expression patterns exist, as is the case in the central nervous system (CNS). However, available RNAi-based gene therapies for the treatment of intractable TLE are limited. Therefore, there exists an urgent need for new therapeutic modalities for the treatment of seizure disorders, such as, e.g., TLE (e.g., TLE refractory to treatment).

### SUMMARY OF THE DISCLOSURE

The present disclosure provides compositions and methods for the treatment or prevention of an epilepsy, such as, e.g., a temporal lobe epilepsy (TLE), in a subject (e.g., a human) in need thereof. The disclosed methods include administration of a therapeutically effective amount of an inhibitory RNA (e.g., an antisense oligonucleotide (ASO, shRNA, siRNA, microRNA, or shmiRNA) that targets an mRNA encoded by a glutamate ionotropic receptor kainate type subunit 2 (Grik2) gene, or a nucleic acid vector encoding the same (e.g., a lentiviral vector or an adeno-associated viral (AAV) vector, such as, e.g., an AAV9 vector), to a subject diagnosed as having or at risk of developing an epilepsy. The disclosure also features pharmaceutical compositions containing one or more of the disclosed ASO agents or nucleic acid vectors encoding the same.

In a first aspect, the disclosure provides an isolated polynucleotide having a length of no more than 800 (e.g., no more than 800, 750, 700, 650, 600, 550, 500, 450, 400, 350, 300, 250, 200, 150, 100, 10 90, 80, 70, 60, 50, 40, 30, 29, 28, 27, 26, 25, 24, 23, 22, 21, 20, or 19) nucleotides that specifically hybridizes within a single-stranded region of a Grik2 mRNA, wherein the hybridized polynucleotide has a Target Opening Energy of less than 18 kcal/mol (e.g., less than 17 kcal/mol, 16 kcal/mol, kcal/mol, 14 kcal/mol, 13 kcal/mol, 12 kcal/mol, 11 kcal/mol, kcal/mol, 9 kcal/mol, 8 kcal/mol, 7 kcal/mol, 6 kcal/mol, kcal/mol, 4 kcal/mol, 3 kcal/mol, 2 kcal/mol, or 1 kcal/mol or less), and wherein: (a) the polynucleotide does not include the nucleic acid sequence of any one of SEQ ID NOs: 772-774 (i.e., SEQ ID NOs: 1-3 of European Patent Application No.: EP19185533.7); (b) the polynucleotide comprises the nucleic acid sequence of SEQ ID NO: 68 or SEQ ID NO: 68 and SEQ ID NO: 649; or (c) the polynucleotide does not have a Total Opening energy that is between 5.53 kcal/mol and 5.55 kcal/mol (e.g., 5.4 kcal/mol).

In some embodiments of the foregoing aspect, the polynucleotide does not include the sequence of any one of SEQ ID NOs: 772-774. In some embodiments, the polynucleotide does not include the sequence of any one of SEQ ID NOs: 772-774 in combination with any one of the sequences of SEQ ID NOs: 1-771. In some embodiments, the nucleic acid sequence of any one of SEQ ID NOs: 772-774 has a Total Opening Energy that is between 5.53 kcal/mol and 5.55 kcal/mol (e.g., 5.4 kcal/mol).

In another aspect, the disclosure provides an isolated RNA polynucleotide having a length of no more than 23 nucleotides that specifically hybridizes within a single-stranded region of a Grik2 mRNA, wherein the hybridized polynucleotide has a Total Opening Energy of less than 18 kcal/mol (e.g., less than 17 kcal/mol, 16 kcal/mol, kcal/mol, 14 kcal/mol, 13 kcal/mol, 12 kcal/mol, 11 kcal/mol, kcal/mol, 9 kcal/mol, 8 kcal/mol, 7 kcal/mol, 6 kcal/mol, kcal/mol, 4 kcal/mol, 3 kcal/mol, 2 kcal/mol, or 1 kcal/mol or less), wherein the polynucleotide does not include the nucleic acid sequence of any one of SEQ ID NOs: 772-774.

In some embodiments of the foregoing aspects, the hybridized polynucleotide does not have a Total Opening energy that is between 5.53 kcal/mol and 5.55 kcal/mol. In some embodiments, the hybridized polynucleotide has a Total Opening Energy that is less than 5.54 kcal/mol. In some embodiments, the hybridized polynucleotide has a Total Opening Energy that is greater than 5.54 kcal/mol. In some embodiments, the hybridized polynucleotide has a Total Opening Energy that is less than 5.54 kcal/mol or greater than 5.54 kcal/mol.

In some embodiments of the foregoing aspects, the hybridized polynucleotide has an Energy of/from Duplex Formation that is greater than −kcal/mol (e.g., greater than −kcal/mol, −kcal/mol, −kcal/mol, −kcal/mol, −kcal/mol, −kcal/mol, −4 kcal/mol, −3 kcal/mol, −2 kcal/mol, −1 kcal/mol or greater). In some embodiments, the hybridized polynucleotide does not have an Energy of Duplex

Formation that is between −36.7 kcal/mol and −36.5 kcal/mol. In some embodiments, the hybridized polynucleotide has an Energy of Duplex Formation that is greater than −36.61 kcal/mol. In some embodiments, the hybridized polynucleotide has an Energy of Duplex Formation that is less than −36.61 kcal/mol.

In some embodiments, the hybridized polynucleotide has a Total Energy of Binding of greater than −24 kcal/mol (e.g., greater than −kcal/mol, −kcal/mol, −kcal/mol, −kcal/mol, −4 kcal/mol, −3 kcal/mol, −2 kcal/mol, −1 kcal/mol or greater). In some embodiments, the hybridized polynucleotide does not have a Total Energy of Binding that is between −29.5 kcal/mol and −29.3 kcal/mol. In some embodiments, the hybridized polynucleotide has a Total Energy of Binding that is greater than −29.4 kcal/mol. In some embodiments, the hybridized polynucleotide has a Total Energy of Binding that is less than −29.4 kcal/mol.

In some embodiments, the hybridized polynucleotide has a GC content that is less than 50% (e.g., less than 45%, 40%, 35%, 30%, 25%, 20%, 15%, 10%, 5%, 4%, 3%, 2%, 1%, or less). In some embodiments, the hybridized polynucleotide does not have a GC content that is between 42.7% and 47.6%. In some embodiments, the hybridized polynucleotide has a GC content that is less than 42.9%. In some embodiments, the hybridized polynucleotide has a GC content that is greater than 42.9%. In some embodiments the GC content is determined for the polynucleotide. In some embodiments, the GC content is determined for a sequence that is substantially complementary (e.g., having no more than 10, 9, 8, 7, 6, 5, 4, 3, 2, or 1 mismatches) to the polynucleotide. In some embodiments, the GC content is determined for a duplex formed by hybridization between the polynucleotide and a sequence that is substantially complementary to the polynucleotide.

In some embodiments, the polynucleotide does not include the sequence of any one of SEQ ID NOs: 772-774 in combination with any one or more of the sequences of SEQ ID NOs: 1-771. In some embodiments, the polynucleotide does not include the sequence of any one of SEQ ID NOs: 772-774 in combination with the sequence of SEQ ID NO: 68. In some embodiments, the polynucleotide does not include the sequence of any one of SEQ ID NOs: 772-774 in combination with the sequence of SEQ ID NO: 68 and 649.

In another aspect, the present disclosure provides an isolated polynucleotide having a length of no more than 800 nucleotides that specifically hybridizes within a single-stranded region of a Grik2 mRNA, wherein the hybridized polynucleotide does not have a Total Opening Energy that is between 5.53 and 5.55 kcal/mol, and wherein the polynucleotide does not include the nucleic acid sequence of any one of SEQ ID NOs: 772-774. In some embodiments, the polynucleotide does not include the sequence of any one of SEQ ID NOs: 772-774 in combination with any one or more of the sequences of SEQ ID NOs: 1-771. In some embodiments, the polynucleotide does not include the sequence of any one of SEQ ID NOs: 772-774 in combination with the sequence of SEQ ID NO: 68. In some embodiments, the polynucleotide does not include the sequence of any one of SEQ ID NOs: 772-774 in combination with the sequence of SEQ ID NO: 68 and 649.

In another aspect, the present disclosure provides an isolated polynucleotide having a length of no more than 800 nucleotides that specifically hybridizes within a single-stranded region of a Grik2 mRNA, wherein the hybridized polynucleotide does not have an Energy of Duplex Formation that is between −36.7 and −36.5 kcal/mol, and wherein the polynucleotide does not include the nucleic acid sequence of any one of SEQ ID NOs: 772-774. In some embodiments, the polynucleotide does not include the sequence of any one of SEQ ID NOs: 772-774 in combination with any one or more of the sequences of SEQ ID NOs: 1-771. In some embodiments, the polynucleotide does not include the sequence of any one of SEQ ID NOs: 772-774 in combination with the sequence of SEQ ID NO: 68. In some embodiments, the polynucleotide does not include the sequence of any one of SEQ ID NOs: 772-774 in combination with the sequence of SEQ ID NO: 68 and 649.

In another aspect, the present disclosure provides an isolated polynucleotide having a length of no more than 800 nucleotides that specifically hybridizes within a single-stranded region of a Grik2 mRNA, wherein the hybridized polynucleotide does not have a Total Energy of Binding that is between −29.5 and −29.3 kcal/mol, and wherein the polynucleotide does not include the nucleic acid sequence of any one of SEQ ID NOs: 772-774. In some embodiments, the polynucleotide does not include the sequence of any one of SEQ ID NOs: 772-774 in combination with any one or more of the sequences of SEQ ID NOs: 1-771. In some embodiments, the polynucleotide does not include the sequence of any one of SEQ ID NOs: 772-774 in combination with the sequence of SEQ ID NO: 68. In some embodiments, the polynucleotide does not include the sequence of any one of SEQ ID NOs: 772-774 in combination with the sequence of SEQ ID NO: 68 and 649.

In another aspect, the present disclosure provides an isolated RNA polynucleotide having a length of no more than 23 nucleotides that specifically hybridizes within a single-stranded region of a Grik2 mRNA, wherein the hybridized polynucleotide does not have a Total Opening Energy that is between 5.53 and 5.55 kcal/mol, and wherein the polynucleotide does not include the nucleic acid sequence of any one of SEQ ID NOs: 772-774. In some embodiments, the polynucleotide does not include the sequence of any one of SEQ ID NOs: 772-774 in combination with any one or more of the sequences of SEQ ID NOs: 1-771. In some embodiments, the polynucleotide does not include the sequence of any one of SEQ ID NOs: 772-774 in combination with the sequence of SEQ ID NO: 68. In some embodiments, the polynucleotide does not include the sequence of any one of SEQ ID NOs: 772-774 in combination with the sequence of SEQ ID NO: 68 and 649.

In another aspect, the present disclosure provides an isolated RNA polynucleotide having a length of no more than 23 nucleotides that specifically hybridizes within a single-stranded region of a Grik2 mRNA, wherein the hybridized polynucleotide does not have an Energy of Duplex Formation that is between −36.7 and −36.5 kcal/mol, and wherein the polynucleotide does not include the nucleic acid sequence of any one of SEQ ID NOs: 772-774. In some embodiments, the polynucleotide does not include the sequence of any one of SEQ ID NOs: 772-774 in combination with any one or more of the sequences of SEQ ID NOs: 1-771. In some embodiments, the polynucleotide does not include the sequence of any one of SEQ ID NOs: 772-774 in combination with the sequence of SEQ ID NO: 68. In some embodiments, the polynucleotide does not include the sequence of any one of SEQ ID NOs: 772-774 in combination with the sequence of SEQ ID NO: 68 and 649.

In another aspect, the present disclosure provides an isolated RNA polynucleotide having a length of no more than 23 nucleotides that specifically hybridizes within a single-stranded region of a

Grik2 mRNA, wherein the hybridized polynucleotide does not have a Total Energy of Binding that is between −29.5 and −29.3 kcal/mol, and wherein the polynucleotide does not include the nucleic acid sequence of any one of SEQ ID NOs: 772-774. In some embodiments, the polynucleotide does not include the sequence of any one of SEQ ID NOs: 772-774 in combination with any one or more of the sequences of SEQ ID NOs: 1-771. In some embodiments, the polynucleotide does not include the sequence of any one of SEQ ID NOs: 772-774 in combination with the sequence of SEQ ID NO: 68. In some embodiments, the polynucleotide does not include the sequence of any one of SEQ ID NOs: 772-774 in combination with the sequence of SEQ ID NO: 68 and 649.

In some embodiments of the foregoing aspects, the single-stranded region of the Grik2 mRNA is selected from the group consisting of Loop regions 1-14. In some embodiments, the polynucleotide specifically hybridizes within: (a) a Loop1 region of the Grik2 mRNA; (b) a Loop2 region of the Grik2 mRNA; (c) a Loop3 region of the Grik2 mRNA; (d) a Loop4 region of the Grik2 mRNA; (e) a Loop region of the Grik2 mRNA; (f) a Loop6 region of the Grik2 mRNA; (g) a Loop7 region of the Grik2 mRNA; (h) a Loop8 region of the Grik2 mRNA; (i) a Loop9 region of the Grik2 mRNA; (j) a Loop region of the Grik2 mRNA; (k) a Loop11 region of the Grik2 mRNA; (I) a Loop12 region of the Grik2 mRNA; (m) a Loop13 region of the Grik2 mRNA; or (n) a Loop14 region of the Grik2 mRNA.

In some embodiments, the Loop1 the region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 145.

In some embodiments, the Loop2 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 146.

In some embodiments, the Loop3 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 147.

In some embodiments, the Loop4 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 148.

In some embodiments, the Loop region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 149.

In some embodiments, the Loop6 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 150.

In some embodiments, the Loop7 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 151.

In some embodiments, the Loop8 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 152.

In some embodiments, the Loop9 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 153.

In some embodiments, the Loop region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 154.

In some embodiments, the Loop11 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 155.

In some embodiments, the Loop12 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 156.

In some embodiments, the Loop13 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 157.

In some embodiments, the Loop14 region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 158.

In some embodiments, the sequence identity is determined over at least (e.g., at least 16, 17, 18, 19, 20, 25, 30, 35, 40, 45, 50, 55, 60, 65, 70, 75, or 80) contiguous nucleotides of any one of SEQ ID NOs: 145-158. In some embodiments, the sequence identity is determined over at least (e.g., at least 35, 40, 45, 50, 55, 60, 65, 70, 75, or 80) contiguous nucleotides of any one of SEQ ID NOs: 145-158. In some embodiments, the sequence identity is determined over at least 60 (e.g., at least 65, 70, 75, or 80) contiguous nucleotides of any one of SEQ ID NOs: 145-158. In some embodiments, the sequence identity is determined over the full length of any one of SEQ ID NOs: 145-158.

In some embodiments, the polynucleotide includes:
(a) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 1;
(b) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 4;
(c) (i) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 5; or
(ii) a nucleic acid sequence having at least 85%,90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 6;
(d) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 7;
(e) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 96;
(f) (i) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 8;
   (ii) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 98; or
   (iii) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 99;
(g) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 9;
(h) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 63;
(i) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 10; or
(j) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 11.

In some embodiments, sequence identity is determined over at least (e.g., at least 11, 12, 13, 14, 15, 16, 17, 18, 19, 20, 21, or 22) contiguous nucleotides of any one of SEQ ID NOs: 1, 4-11, 63, 96, 98, or 99. In some embodiments, sequence identity is determined over at least (e.g., at least 16, 17, 18, 19, 20, 21, or 22) contiguous nucleotides of any one of SEQ ID NOs: 1, 4-11, 63, 96, 98, or 99. In some embodiments, sequence identity is determined over at least (e.g., at least 20, 21, or 22) contiguous nucleotides of any one of SEQ ID NOs: 1, 4-11, 63, 96, 98, or 99. In some embodiments, sequence identity is determined over the full length of any one of SEQ ID NOs: 1, 4-11, 63, 96, 98, or 99.

In some embodiments, the polynucleotide comprises a duplex structure formed by the polynucleotide and the single-stranded region of the Grik2 mRNA, wherein the duplex structure comprises at least one (e.g., at least 2, 3, 4, 5, 6, 7, 8, 9, 10, 11, 12, 13, 14, 15 or more) mismatch between the nucleotides of the polynucleotide and nucleotides of the single-stranded region of the Grik2 mRNA.

In some embodiments, the single-stranded region of the Grik2 mRNA is selected from the group consisting of Loop regions 1-14.

In some embodiments, the average positional entropy is calculated over 23 to 79 nucleotides.

In some embodiments, the single-stranded region of the Grik2 mRNA is selected from the group consisting of Unpaired regions 1-5.

In some embodiments, the polynucleotide specifically hybridizes within (a) a Unpaired region 1 of the Grik2 mRNA; (b) a Unpaired region 2 of the Grik2 mRNA; (c) a Unpaired region 3 of the Grik2 mRNA; (d) a Unpaired region 4 of the Grik2 mRNA; or (e) a Unpaired region of the Grik2 mRNA.

In some embodiments, (a) the Unpaired region 1 is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 159; (b) the Unpaired region 2 is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 160; (c) the Unpaired region 3 is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 161; (d) the Unpaired region 4 is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 162;

and/or (e) the Unpaired region is encoded by a nucleic acid sequence having at least 85%, 90%, 92%, 95%, 97%, 99%, or 100% sequence identity over at least (e.g., at least 11, 12, 13, 14, 15, 16, 17, 18, 19, 20, 25, 30, 35, 40, 45, 50, 55, 60, 65, 70, 75, or 80) contiguous nucleotides of SEQ ID NO: 163.

In some embodiments, sequence identity is determined over at least (e.g., at least 16, 17, 18, 19, 20, 25, 30, 35, 40, 45, 50, 55, 60, 65, 70, 75, or 80) contiguous nucleotides of any one of SEQ ID NOs: 159-163. In some embodiments, sequence identity is determined over at least (e.g., at least 35, 40, 45, 50, 55, 60, 65, 70, 75, or 80) contiguous nucleotides of any one of SEQ ID NOs: 159-163. In some embodiments, sequence identity is determined over at least 60 (e.g., at least 65, 70, 75, or 80) contiguous nucleotides of any one of SEQ ID NOs: 159-163. In some embodiments, sequence identity is determined over the full length of any one of SEQ ID NOs: 159-163.

In some embodiments, the polynucleotide includes:
(a) (i) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 13;
   (ii) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 14;
   (iii) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 40 18, 19, 20, 21, or 22) contiguous nucleotides of SEQ ID NO: 72; or
   (iv) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 73;
(b) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 15; or
(c) a nucleic acid sequence having at least 85%, 90%, 92%, 95%, 97%, 99%, or 100% sequence identity over at least (e.g., at least 6, 7, 8, 9, 10, 11, 12, 13, 14, 15, 16, 17, 18, 19, 20, 21, or 22) contiguous nucleotides of SEQ ID NO: 16.

In some embodiments, sequence identity is determined over at least (e.g., at least 10, 11, 12, 13, 14, 15, 16, 17, 18, 19, 20, 21, or 22) contiguous nucleotides of any one of SEQ ID NOs: 13-16, 72 or 73. In some embodiments, sequence identity is determined over at least (e.g., at least 15, 16, 17, 18, 19, 20, 21, or 22) contiguous nucleotides of any one of SEQ ID NOs: 13-16, 72 or 73. In some embodiments, sequence identity is determined over at least (e.g., at least 20, 21, or 22) contiguous nucleotides of any one of SEQ ID NOs: 13-16, 72 or 73. In some embodiments, sequence identity is determined over the full length of any one of SEQ ID NOs: 13-16, 72 or 73. In some embodiments, sequence identity is determined over no more than (e.g., no more than 30, 25, 20, 15, 10, 9, 8, 7, 6, 5, 4, 3, or 2) contiguous nucleotides of any one of SEQ ID NOs: 13-16, 72, or 73. In some embodiments, sequence identity is determined over no more than (e.g., no more than 25, 20, 15, 10, 9, 8, 7, 6, 5, 4, 3, or 2) contiguous nucleotides of any one of SEQ ID NOs: 13-16, 72, or 73.

In some embodiments, the polynucleotide comprises a duplex structure formed by the polynucleotide and the single-stranded region of the Grik2 mRNA, wherein the duplex structure comprises at least one (e.g., at least 1, 2, 3, 4, 5, 6, 7, 8, 9, 10, 11, 12, 13, 14, 15 or more) mismatch between the nucleotides of the polynucleotide and nucleotides of the single-stranded region of the Grik2 mRNA.

In some embodiments, the average positional entropy is calculated over 23 to 79 nucleotides. In some embodiments, the polynucleotide hybridizes to a coding sequence of the Grik2 mRNA. In some embodiments, the polynucleotide hybridizes to (a) a region within exon 1 of the Grik2 mRNA; (b) a region within exon 2 of the Grik2 mRNA; (c) a region within exon 3 of the Grik2 mRNA; (d) a region within exon 4 of the Grik2 mRNA; (e) a region within exon of the Grik2 mRNA; (f) a region within exon 6 of the Grik2 mRNA; (g) a region within exon 7 of the Grik2 mRNA; (h) a region within exon 8 of the Grik2 mRNA; (i) a region within exon 9 of the Grik2 mRNA; (j) a region within exon of the Grik2 mRNA; (k) a region within exon 11 of the Grik2 mRNA; (I) a region within exon 12 of the Grik2 mRNA; (m) a region within exon 13 of the Grik2 mRNA; (n) a region within exon 14 of the Grik2 mRNA; (o) a region within exon of the Grik2 mRNA; and/or (p) a region within exon 16 of the Grik2 mRNA.

In some embodiments, (a) exon 1 of the Grik2 mRNA is encoded by a nucleic acid sequence having at least 85%, 90%, 92%, 95%, 97%, 99%, or 100% sequence identity over at least contiguous nucleotides of SEQ ID NO: 129; (b) exon 2 of the Grik2 mRNA is encoded by a nucleic acid sequence having at least 85%, 90%, 92%, 95%, 97%, 99%, or 100% sequence identity over at least contiguous nucleotides of SEQ ID NO: 130; (c) exon 3 of the Grik2 mRNA is encoded by a nucleic acid sequence having at least 85%, 90%, 92%, 95%, 97%, 99%, or 100% sequence identity over at least contiguous nucleotides of SEQ ID NO: 131; (d) exon 4 of the Grik2 mRNA is encoded by a nucleic acid sequence having at least 85%, 90%, 92%, 95%, 97%, 99%, or 100% sequence identity over at least contiguous nucleotides of SEQ ID NO: 132; (e) exon of the Grik2 mRNA is encoded by a nucleic acid sequence having at least 85%, 90%, 92%, 95%, 97%, 99%, or 100% sequence identity over at least contiguous nucleotides of SEQ ID NO: 133; (f) exon 6 of the Grik2 mRNA is encoded by a nucleic acid sequence having at least 85%, 90%, 92%, 95%, 97%, 99%, or 100% sequence identity over at least contiguous nucleotides of SEQ ID NO: 134; (g) exon 7 of the Grik2 mRNA is encoded by a nucleic acid sequence having at least 85%, 90%, 92%, 95%, 97%, 99%, or 100% sequence identity over at least contiguous nucleotides of SEQ ID NO: 135; (h) exon 8 of the Grik2 mRNA is encoded by a nucleic acid sequence having at least 85%, 90%, 92%, 95%, 97%, 99%, or 100% sequence identity over at least contiguous nucleotides of SEQ ID NO: 136; (i) exon 9 of the Grik2 mRNA is encoded by a nucleic acid sequence having at least 85%, 90%, 92%, 95%, 97%, 99%, or 100% sequence identity over at least contiguous nucleotides of SEQ ID NO: 137; (j) exon of the Grik2 mRNA is encoded by a nucleic acid sequence having at least 85%, 90%, 92%, 95%, 97%, 99%, or 100% sequence identity over at least contiguous nucleotides of SEQ ID NO: 138; (k) exon 11 of the Grik2 mRNA is encoded by a nucleic acid sequence having at least 85%, 90%, 92%, 95%, 97%, 99%, or 100% sequence identity over at least contiguous nucleotides of SEQ ID NO: 139; (I) exon 12 of the Grik2 mRNA is encoded by a nucleic acid sequence having at least 85%, 90%, 92%, 95%, 97%, 99%, or 100% sequence identity over at least contiguous nucleotides of SEQ ID NO: 140; (m) exon 13 of the Grik2 mRNA is encoded by a nucleic acid sequence having at least 85%, 90%, 92%, 95%, 97%, 99%, or 100% sequence identity over at least contiguous nucleotides of SEQ ID NO: 141; (n) exon 14 of the Grik2 mRNA is encoded by a nucleic acid sequence having at least 85%, 90%, 92%, 95%, 97%, 99%, or 100% sequence identity over at least contiguous nucleotides of SEQ ID NO: 142; (o) exon of the Grik2 mRNA is encoded by a nucleic acid sequence having at least 85%, 90%, 92%, 95%, 97%, 99%, or 100% sequence identity over at least contiguous nucleotides of SEQ ID NO: 143; and/or (p) exon 16 of the Grik2 mRNA is encoded by a nucleic acid sequence having at least 85%, 90%, 92%, 95%, 97%, 99%, or 100% sequence identity over at least contiguous nucleotides of SEQ ID NO: 144.

In some embodiments, the polynucleotide comprises:
(a) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 1;
(b) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 2;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 3;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 30;
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 31;
   (v) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 36;
   (vi) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 40;
   (vii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 59;
   (viii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 76;
   (ix) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 80;
   (x) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 81;
   (xi) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 92; and/or
   (xii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 93;
(c) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 40;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 60;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 68;
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 70; and/or
   (v) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 86;
(d) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 68;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 69; and/or
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 70;
(e) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 4;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 5;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 6;
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 56;
   (v) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 57;
   (vi) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 58;
   (vii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 91;
   (viii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 94; and/or
   (ix) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 95;
(f) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 20;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 37;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 38;
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 44;
   (v) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 46;
(g) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 12;
(h) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 7;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 8;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 96;
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 98; and/or
   (v) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 99;
(i) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 22;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 39;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 62;
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 74;
   (v) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 75;
   (vi) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 87;
   (vii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 88;
   (viii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 89; and/or
   (ix) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 90;
(j) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 82;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 83;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 84; and/or
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 85;
(k) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 13;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 14;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 72; and/or
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 73;
(I) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 34;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 35;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 77;
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 78; and/or
   (v) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 79;
(m) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 51;
and/or
(n) (i) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 9;
   (ii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 10;
   (iii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 11;
   (iv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 15;
   (v) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 25 18, 19, 20, 21, or 22) contiguous nucleotides of SEQ ID NO: 16;
   (vi) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 17;
   (vii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 18;
   (viii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 27;
   (ix) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 32;
   (x) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 33;
   (xi) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 41;
   (xii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 49;
   (xiii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 50;
   (xiv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 52;
   (xv) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 53;
   (xvi) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 61; and/or
   (xvii) a nucleic acid sequence having at least 85%, 90%, 92%, 95%, 97%, 99%, or 100% sequence identity over at least (e.g., at least 5, 6, 7, 8, 9, 10, 11, 12, 13, 14, 15, 16, 17, 18, 19, 20, 21, or 22) contiguous nucleotides of SEQ ID NO: 63.

In some embodiments, sequence identity is determined over at least (e.g., at least 10, 11, 12, 13, 14, 15, 16, 17, 18, 19, 20, 21, or 22) contiguous nucleotides of any one of SEQ ID NOs: 1-12, 13-18, 20, 22, 27, 30-41, 44, 46, 49-53, 56-63, 68-70, 72-92, or 94-99. In some embodiments, sequence identity is determined over at least (e.g., at least 15, 16, 17, 18, 19, 20, 21, or 22) contiguous nucleotides of any one of SEQ ID NOs: 1-12, 13-18, 20, 22, 27, 30-41, 44, 46, 49-53, 56-63, 68-70, 72-92, or 94-99. In some embodiments, sequence identity is determined over at least (e.g., at least 20, 21, or 22) contiguous nucleotides of any one of SEQ ID NOs: 1-12, 13-18, 20, 22, 27, 30-41, 44, 46, 49-53, 56-63, 68-70, 72-92, or 94-99. In some embodiments, sequence identity is determined over the full length of any one of SEQ ID NOs: 1-12, 13-18, 20, 22, 27, 30-41, 44, 46, 49-53, 56-63, 68-70, 72-92, or 94-99.

In some embodiments, the polynucleotide comprises a nucleic acid sequence of SEQ ID NO: 68. In some embodiments, the polynucleotide comprises a nucleic acid sequence of SEQ ID NO: 68 and 649. In some embodiments, the polynucleotide comprises from to 3′: a nucleic acid sequence of SEQ ID NO: 68, 758, and 649. In some embodiments, the polynucleotide comprises from to 3′: a nucleic acid sequence of SEQ ID NO: 649, 758, and 68. In some embodiments, the polynucleotide comprises from to 3′: a nucleic acid sequence of SEQ ID NO: 649, 758, and 68. In some embodiments, the polynucleotide comprises from to 3′: a nucleic acid sequence of SEQ ID NO: 752, 68, 758, 649 and 753. In some embodiments, the polynucleotide comprises from to 3′: a nucleic acid sequence of SEQ ID NO: 752, 649, 758, 68, and 753.

In some embodiments, the polynucleotide hybridizes to a non-coding sequence of the Grik2 mRNA. In some embodiments, the non-coding sequence includes a 5′ untranslated region (UTR) of the Grik2 mRNA. In some embodiments, the 5′ UTR is encoded by a polynucleotide having at least 85% (e.g., at least 86%, 90%, 95%, 96%, 97%, 98%, 99%, or more) sequence identity to the nucleic acid sequence of SEQ ID NO: 126. In some embodiments, the non-coding sequence comprises a 3′ UTR of the Grik2 mRNA. In some embodiments, the 3′ UTR is encoded by a polynucleotide having at least 85% (e.g., at least 86%, 90%, 95%, 96%, 97%, 98%, 99%, or more) sequence identity to the nucleic acid sequence of SEQ ID NO: 127.

In some embodiments, the polynucleotide hybridizes to any one of the nucleic acid sequences of SEQ ID NOs: 115-681.

In some embodiments, the polynucleotide has at least 85% (e.g., at least 86%, 90%, 95%, 96%, 97%, 98%, 99%, or more) sequence identity to the nucleic acid sequence of any one of SEQ ID NOs: 1-100.

In some embodiments, the polynucleotide is an antisense oligonucleotide (ASO). In some embodiments, the ASO is a short interfering RNA (siRNA), a short hairpin RNA (shRNA), a microRNA (miRNA), or an shRNA-adapted microRNA (shmiRNA).

In some embodiments, the polynucleotide is between 19-21 nucleotides. In some embodiments, the polynucleotide is 19 nucleotides. In some embodiments, the polynucleotide is nucleotides. In some embodiments, the polynucleotide is 21 nucleotides.

In some embodiments, the Grik2 mRNA is encoded by a nucleic acid sequence of SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, or SEQ ID NO: 124.

In some embodiments, the polynucleotide is capable of reducing a level of Gluk2 protein in a cell.

In some embodiments, the polynucleotide reduces a level of GluK2 protein in the cell by at least 10%, at least at least 15%, at least 20%, at least 25%, at least 30%, at least 35%, at least 40%, at least 45%, at least 50%, at least 55%, at least 60%, at least 65%, at least 70%, or at least 75%. In some embodiments, the cell is a neuron. In some embodiments, the neuron is a hippocampal neuron. In some embodiments, the hippocampal neuron is a dentate granule cell (DGC).

In some embodiments of the foregoing aspects, the polynucleotide does not include the sequence of any one of SEQ ID NOs: 772-774. In some embodiments, the polynucleotide does not include the sequence of any one of SEQ ID NOs: 772-774 in combination with any one of the sequences of any one of SEQ ID NOs: 1-771. In some embodiments, the polynucleotide does not include the sequence of any one of SEQ ID NOs: 772-774 in combination with the sequence of SEQ ID NO: 68. In some embodiments, the polynucleotide does not include the sequence of any one of SEQ ID NOs: 772-774 in combination with the sequence of SEQ ID NO: 68 and 649.

In another aspect, the disclosure provides a vector comprising the polynucleotide of the foregoing aspect and embodiments. In some embodiments, the vector is replication-defective. In some embodiments, the replication-defective vector is a vector lacking one or more coding regions of genes necessary for virion synthesis, replication, and packaging. In some embodiments, the vector is a mammalian, bacterial, or viral vector. In some embodiments, the vector is an expression vector.

In some embodiments, the viral vector is selected from the group consisting of an adeno-associated virus (AAV), retrovirus, adenovirus, parvovirus, coronavirus, negative strand RNA viruses, orthomyxovirus, rhabdovirus, paramyxovirus, positive strand RNA viruses, picornavirus, alphavirus, a double stranded DNA virus, herpesvirus, Epstein-Barr virus, cytomegalovirus, fowlpox virus, and canarypox virus. In some embodiments, the vector is an AAV vector. In some embodiments, the AAV vector is an AAV9 or AAVrhvector.

In some embodiments, the vector includes an expression cassette containing any one of the sequences defined in Table 9 or Table of U.S. Provisional Patent Application No.: 63/050,742, which is incorporated herein by reference.

In some embodiments, the vector of the foregoing aspect does not include the sequence of any one of SEQ ID NOs: 772-774. In some embodiments, the vector does not include the sequence of any one of SEQ ID NOs: 772-774 in combination with any one or more of the sequences of SEQ ID NOs: 1-771. In some embodiments, the vector does not include the sequence of any one of SEQ ID NOs: 772-774 in combination with the sequence of SEQ ID NO: 68. In some embodiments, the vector does not include the sequence of any one of SEQ ID NOs: 772-774 in combination with the sequence of SEQ ID NO: 68 and 649.

In another aspect, the disclosure provides an expression cassette including a hSyn promoter (e.g., any one of SEQ ID NOs: 682-685 and 790 or a variant thereof with up to 85% or more sequence identity thereto) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another aspect, the disclosure provides an expression cassette including a CaMKII promoter (e.g., any one of SEQ ID NOs: 687-691 and 802 or a variant thereof with up to 85% or more sequence identity thereto) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another aspect, the disclosure provides an expression cassette including a CAG promoter (e.g., SEQ ID NO: 737 or a variant thereof with up to 85% or more sequence identity thereto) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another aspect, the disclosure provides an expression cassette including a CBA promoter (e.g., SEQ ID NO: 738 or a variant thereof with up to 85% or more sequence identity thereto) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another aspect, the disclosure provides an expression cassette including a U6 promoter (e.g., any one of SEQ ID NOs: 728-733 or a variant thereof with up to 85% or more sequence identity thereto) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another aspect, the disclosure provides an expression cassette including a H1 promoter (e.g., SEQ ID NO: 734 or a variant thereof with up to 85% or more sequence identity thereto) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another aspect, the disclosure provides an expression cassette including a 7SK promoter (e.g., SEQ ID NO: 746 or a variant thereof with up to 85% or more sequence identity thereto) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another aspect, the disclosure provides an expression cassette including a hSyn promoter (e.g., any one of SEQ ID NOs: 682-685 and 790 or a variant thereof with up to 85% or more sequence identity thereto) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another aspect, the disclosure provides an expression cassette including a CaMKII promoter (e.g., any one of SEQ ID NOs: 687-691 and 802 or a variant thereof with up to 85% or more sequence identity thereto) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another aspect, the disclosure provides an expression cassette including a CAG promoter (e.g., SEQ ID NO: 737 or a variant thereof with up to 85% or more sequence identity thereto) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another aspect, the disclosure provides an expression cassette including a CBA promoter (e.g., SEQ ID NO: 738 or a variant thereof with up to 85% or more sequence identity thereto) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another aspect, the disclosure provides an expression cassette including a U6 promoter (e.g., any one of SEQ ID NOs: 728-733 or a variant thereof with up to 85% or more sequence identity thereto) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another aspect, the disclosure provides an expression cassette including a H1 promoter (e.g., SEQ ID NO: 734 or a variant thereof with up to 85% or more sequence identity thereto) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another aspect, the disclosure provides an expression cassette including a 7SK promoter (e.g., SEQ ID NO: 746 or a variant thereof with up to 85% or more sequence identity thereto) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence. In another aspect, the disclosure provides an expression cassette selected from any one of the expression cassettes described in Table 9 (see Detailed Description).

In another aspect, the disclosure provides an expression cassette including a nucleotide sequence containing a stem-loop sequence comprising, from 5′ to 3′: (i) a 5′ stem-loop arm comprising a guide nucleotide sequence having at least 85% (e.g., at least 86%, 90%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a loop region, wherein the loop region comprises a microRNA loop sequence; (iii) a 3′ stem-loop arm comprising a passenger nucleotide sequence that is complementary or substantially complementary to the guide sequence.

In another aspect, the disclosure provides an expression cassette including a nucleotide sequence containing: (a) a stem-loop sequence comprising, from 5′ to 3′: (i) a 5′ stem-loop arm comprising a guide nucleotide sequence having at least 85% (e.g., at least 86%, 90%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a loop region, wherein the loop region comprises a microRNA loop sequence; (iii) a 3′ stem-loop arm comprising a passenger nucleotide sequence that is complementary or substantially complementary to the guide sequence, (b) a first flanking region located 5′ to said guide sequence; and (c) a second flanking region located 3′ to said passenger sequence.

In some embodiments, the expression cassette of the foregoing aspect does not include the sequence of any one of SEQ ID NOs: 772-774. In some embodiments, the expression cassette does not include the sequence of any one of SEQ ID NOs: 772-774 in combination with any one or more of the sequences of SEQ ID NOs: 1-771. In some embodiments, the expression cassette does not include the sequence of any one of SEQ ID NOs: 772-774 in combination with the sequence of SEQ ID NO: 68. In some embodiments, the expression cassette does not include the sequence of any one of SEQ ID NOs: 772-774 in combination with the sequence of SEQ ID NO: 68 and 649. In some embodiments, the expression cassette does not include sequence of any one of SEQ ID NOs: 772-774 in combination with the sequence of SEQ ID NO: 68 and 649.

In another aspect, the disclosure provides an expression cassette comprising a nucleotide sequence comprising a stem-loop sequence comprising, from 5′ to 3′: (i) a 5′ stem-loop arm comprising a passenger nucleotide sequence which is complementary or substantially complementary to a guide sequence; (ii) a loop region, wherein the loop region comprises a microRNA loop sequence; (iii) a 3′ stem-loop arm comprising a guide nucleotide sequence having at least 85% (e.g., at least 86%, 90%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto). In some embodiments, the expression cassette further includes a second stem-loop sequence comprising from 5′ to 3′: (i) a second 5′ stem-loop arm comprising a second passenger nucleotide sequence which is complementary or substantially complementary to a second guide sequence; (ii) a second loop region, wherein the second loop region comprises a second microRNA loop sequence; (iii) a second 3′ stem-loop arm comprising a second guide nucleotide sequence having at least 85% (e.g., at least 86%, 90%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto). In some embodiments, the first stem-loop sequence and the second stem-loop sequence are identical. In some embodiments, the first stem-loop sequence and the second stem-loop sequence are different.

In another aspect, the disclosure provides an expression cassette comprising a nucleotide sequence comprising: (a) a stem-loop sequence comprising, from 5′ to 3′: (i) a 5′ stem-loop arm comprising a passenger nucleotide sequence which is complementary or substantially complementary to a guide sequence; (ii) a loop region, wherein the loop region comprises a microRNA loop sequence; (iii) a 3′ stem-loop arm comprising a guide nucleotide sequence having at least 85% (e.g., at least 86%, 90%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (b) a first flanking region located 5′ to said guide sequence; and (c) a second flanking region located 3′ to said passenger sequence. In some embodiments, the expression cassette further includes: (a) a second stem-loop sequence comprising from 5′ to 3′: (i) a second 5′ stem-loop arm comprising a second passenger nucleotide sequence which is complementary or substantially complementary to a second guide sequence; (ii) a second loop region, wherein the second loop region comprises a second microRNA loop sequence; (iii) a second 3′ stem-loop arm comprising a second guide nucleotide sequence having at least 85% (e.g., at least 86%, 90%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (b) a third flanking region located 5′ to said second guide sequence; and (c) a fourth flanking region located 3′ to said second passenger sequence. In some embodiments, the first stem-loop sequence and the second stem-loop sequence are identical. In some embodiments, the first stem-loop sequence and the second stem-loop sequence are different.

In some embodiments of the foregoing aspects and embodiments, the first flanking region comprises a polynucleotide having at least 85% (e.g., at least 86%, 90%, 95%, 96%, 97%, 98%, 99%, or more) sequence identity to the nucleic acid sequence of any one of SEQ ID NOs: 752, 754, 756, 759, 762, 765, or 768. In some embodiments, the second flanking region comprises a polynucleotide having at least 85% (e.g., at least 86%, 90%, 95%, 96%, 97%, 98%, 99%, or more) sequence identity to the nucleic acid sequence of any one of SEQ ID NOs: 753, 755, 757, 760, 763, 766, or 769.

In some embodiments, the first flanking region includes a 5′ spacer sequence and a 5′ flanking sequence. In some embodiments, the second flanking region includes a 3′ spacer sequences and a 3′ flanking sequence.

In some embodiments, the microRNA loop sequence is a miR-30, miR-155, miR-218-1, or miR-124-3 sequence. In some embodiments, the microRNA loop sequence comprises a polynucleotide having at least 85% (e.g., at least 86%, 90%, 95%, 96%, 97%, 98%, 99%, or more) sequence identity to the nucleic acid sequence of any one of SEQ ID NOs: 758, 761, 764, 767, or 770.

In some embodiments, the expression cassette includes a promoter selected from the group consisting of a U6 promoter, H1 promoter, 7SK promoter, Apolipoprotein E-Human Alpha 1-Antitrypsin (ApoE-hAAT) promoter, CAG promoter, CBA promoter, CK8 promoter, mU1 a promoter, Elongation Factor 1a (EF1a) promoter, herpes simplex virus (HSV) promoter Thyroxine Binding Globulin (TBG) promoter, Synapsin promoter (SYN), RNA Binding Fox-1 Homolog 3 (RBFOX3) promoter, Calcium/Calmodulin Dependent Protein Kinase II (CaMKII) promoter, neuron-specific enolase (NSE) promoter, Platelet Derived Growth Factor Subunit β (PDGF β) promoter , Vesicular Glutamate Transporter (VGAT) promoter, Somatostatin (SST) promoter, Neuropeptide Y (NPY) promoter, Vasoactive Intestinal Peptide (VIP) promoter, Parvalbumin (PV) promoter, Glutamate Decarboxylase 65 (GAD65) promoter, Glutamate Decarboxylase 67 (GAD67) promoter, Dopamine Receptor D1 (DRD1) promoter, Dopamine Receptor D2 (DRD2) promoter, Complement C1q Like 2 (C1QL2) promoter, Proopiomelanocortin

(POMC) promoter, Prospero Homeobox 1 (PROX1) promoter, Microtubule Associated Protein 1B (MAP1B) promoter, and Tubulin Alpha 1 (T-α1/TUBA3) promoter. In some embodiments, the expression cassette includes a SYN promoter (e.g., such as a human SYN promoter, e.g., any one of SEQ ID NOs: 682-685 and 790 or a variant thereof having at least 85% (e.g., at least 86%, 90%, 95%, 96%, 97%, 98%, 99%, or more) sequence identity to the nucleic acid sequence of any one of SEQ ID NOs: 682-682 and 790). In some embodiments, the expression cassette includes a CAMKII promoter (e.g., any one of SEQ ID NOs: 687-691 and 802 or a variant thereof having at least 85% (e.g., at least 86%, 90%, 95%, 96%, 97%, 98%, 99%, or more) sequence identity to the nucleic acid sequence of any one of SEQ ID NOs: 687-691 and 802). In some embodiments, the expression cassette includes a C1QL2 promoter (e.g., SEQ ID NO: 719 or SEQ ID NO: 791 or a variant thereof having at least 85% (e.g., at least 86%, 90%, 95%, 96%, 97%, 98%, 99%, or more) sequence identity to the nucleic acid sequence of SEQ ID NO: 719 or SEQ ID NO: 791). In some embodiments, the promoter is operably linked to two or more stem-loop sequences. In some embodiments, the promoter is operably linked to two stem-loop sequences (e.g., two stem-loop sequences that are present in the vector in tandem).

In some embodiments, the expression cassette includes a polynucleotide having at least 85% (e.g., at least 86%, 90%, 95%, 96%, 97%, 98%, 99%, or more) sequence identity to the nucleic acid sequence of any one of SEQ ID NOs: 775, 777, 779, 781, 783-788, 796, 798-801, 803, 805, 807, 809, 811, 813, 817, 819, and 821. In some embodiments, the expression cassette is incorporated into a vector having at least 85% (e.g., at least 86%, 90%, 95%, 96%, 97%, 98%, 99%, or more) sequence identity to the nucleic acid sequence of any one of SEQ ID NOs: 804, 806, 808, 810, 812, 814, 818, 820, and 822.

In another aspect, the disclosure provides an expression cassette comprising, from 5′ to 3′: (a) a first promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (b) a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto), wherein the first guide nucleotide sequence is operably linked to the first promoter; (c) optionally, a second promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (b) a second guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto), wherein, optionally, the second guide nucleotide sequence is operably linked to the second promoter. In some embodiments, the first guide sequence and/or the second guide sequence is a G9 sequence (SEQ ID NO: 68) or a variant thereof with at least 85% (at least 86%, 87%, 88%, 89%, 90%, 91%, 92%, 93%, 94%, 95%, 96%, 97%, 98%, 99%, or more) sequence identity thereto.

In some embodiments, the first guide sequence and/or the second guide sequence is a GI sequence (SEQ ID NO: 77) or a variant thereof with at least 85% (at least 86%, 87%, 88%, 89%, 90%, 91%, 92%, 93%, 94%, 95%, 96%, 97%, 98%, 99%, or more) sequence identity thereto. In some embodiments, the first guide sequence and/or the second guide sequence is a MW sequence (SEQ ID NO: 80) or a variant thereof with at least 85% (at least 86%, 87%, 88%, 89%, 90%, 91%, 92%, 93%, 94%, 95%, 96%, 97%, 98%, 99%, or more) sequence identity thereto. In some embodiments, the first guide sequence and/or the second guide sequence is a MU sequence (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto. In some embodiments, the first guide sequence is a G9 sequence (SEQ ID NO: 68) or a variant thereof with at least 85% (at least 86%, 87%, 88%, 89%, 90%, 91%, 92%, 93%, 94%, 95%, 96%, 97%, 98%, 99%, or more) sequence identity thereto and the second guide sequence is a GI sequence (SEQ ID NO: 77) or a variant thereof with at least 85% (at least 86%, 87%, 88%, 89%, 90%, 91%, 92%, 93%, 94%, 95%, 96%, 97%, 98%, 99%, or more) sequence identity thereto. In some embodiments, the first guide sequence is a G9 sequence (SEQ ID NO: 68 or a variant thereof with at least 85% (at least 86%, 87%, 88%, 89%, 90%, 91%, 92%, 93%, 94%, 95%, 96%, 97%, 98%, 99%, or more) sequence identity thereto and the second guide sequence is a MW sequence (SEQ ID NO: 80) or a variant thereof with at least 85% (at least 86%, 87%, 88%, 89%, 90%, 91%, 92%, 93%, 94%, 95%, 96%, 97%, 98%, 99%, or more) sequence identity thereto. In some embodiments, the first guide sequence is a GI sequence (SEQ ID NO: 77) or a variant thereof with at least 85% (at least 86%, 87%, 88%, 89%, 90%, 91%, 92%, 93%, 94%, 95%, 96%, 97%, 98%, 99%, or more) sequence identity thereto and the second guide sequence is a G9 sequence (SEQ ID NO: 68) or a variant thereof with at least 85% (at least 86%, 87%, 88%, 89%, 90%, 91%, 92%, 93%, 94%, 95%, 96%, 97%, 98%, 99%, or more) sequence identity thereto. In some embodiments, the first guide sequence is a GI sequence (SEQ ID NO: 77) or a variant thereof with at least 85% (at least 86%, 87%, 88%, 89%, 90%, 91%, 92%, 93%, 94%, 95%, 96%, 97%, 98%, 99%, or more) sequence identity thereto and the second guide sequence is a MW sequence (SEQ ID NO: 80) or a variant thereof with at least 85% (at least 86%, 87%, 88%, 89%, 90%, 91%, 92%, 93%, 94%, 95%, 96%, 97%, 98%, 99%, or more) sequence identity thereto.

In some embodiments, the expression cassette includes a polynucleotide having at least 85% (at least 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 785-788.

In some embodiments, the first promoter is a SYN promoter (e.g., any one of SEQ ID NOs: 682-685 and 790 or a variant thereof having at least 85% (e.g., at least 86%, 90%, 95%, 96%, 97%, 98%, 99%, or more) sequence identity thereto and, optionally, the second promoter is a CAMKII promoter (e.g., any one of SEQ ID NOs: 687-691 and 802 or a variant thereof having at least 85% (e.g., at least 86%, 90%, 95%, 96%, 97%, 98%, 99%, or more) sequence identity thereto.

In some embodiments of the foregoing aspect, the expression cassette further includes a first passenger nucleotide sequence which is complementary or substantially complementary to the first guide nucleotide sequence, wherein the first passenger nucleotide sequence is located 5′ or 3′ relative to the first guide nucleotide sequence.

In some embodiments of the foregoing aspect, the expression cassette further includes a second passenger nucleotide sequence which is complementary or substantially complementary to the second guide nucleotide sequence, wherein the second passenger nucleotide sequence is located 5′ or 3′ relative to the second guide nucleotide sequence.

In some embodiments of the foregoing aspect, the expression cassette further includes a first 5′ flanking region (e.g., any one of SEQ ID NOs: 752, 754, 756, 759, 762, 765, and 768 or a variant thereof with at least 85% (at least 86%, 87%, 88%, 89%, 90%, 91%, 92%, 93%, 94%, 95%, 96%, 97%, 98%, 99%, or more) sequence identity thereto) located 5′ relative to the first guide sequence.

In some embodiments of the foregoing aspect, the expression cassette further includes a first 3′ flanking region located 3′ relative to the first guide sequence.

In some embodiments of the foregoing aspect, the expression cassette further includes a second 5′ flanking region located 5′ relative to the second guide sequence.

In some embodiments of the foregoing aspect, the expression cassette further includes a second 3′ flanking region located 3′ relative to the second guide sequence.

In some embodiments of the foregoing aspect, the expression cassette further includes a first loop region located between the first guide sequence and the first passenger sequence, wherein the first loop region comprises a first microRNA loop sequence (e.g., any one of SEQ ID NOs: 758, 761, 764, 767, and 770 or a variant thereof with one, two, or three nucleotide changes thereto).

In some embodiments of the foregoing aspect, the expression cassette further includes a second loop region located between the second guide sequence and the second passenger sequence, wherein the second loop region comprises a second microRNA loop sequence (e.g., any one of SEQ ID NOs: 758, 761, 764, 767, and 770 or a variant thereof with one, two, or three nucleotide changes thereto).

In another aspect, the disclosure provides an expression cassette that includes a nucleotide sequence comprising, from 5′ to 3′:
(a) a first promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto);
(b) a first 5′ flanking region located 5′ to a first passenger nucleotide sequence (e.g., any one of SEQ ID NOs: 752, 754, 756, 759, 762, 765, and 768 or a variant thereof with at least 85% (at least 86%, 87%, 88%, 89%, 90%, 91%, 92%, 93%, 94%, 95%, 96%, 97%, 98%, 99%, or more) sequence identity thereto);
(c) a first stem-loop sequence comprising, from 5′ to 3′:
   (i) a first 5′ stem-loop arm comprising the first passenger nucleotide sequence which is complementary or substantially complementary to a first guide sequence;
   (ii) a first loop region, wherein the first loop region comprises a first microRNA loop sequence (e.g., any one of SEQ ID NOs: 758, 761, 764, 767, and 770 or a variant thereof with one, two, or three nucleotide changes thereto);
   (iii) a first 3′ stem-loop arm comprising a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
(d) a first 3′ flanking region located 3′ to said first guide nucleotide sequence (e.g., any one of SEQ ID NOs: 753, 755, 757, 760, 763, 766, and 769 or a variant thereof with at least 85% (at least 86%, 87%, 88%, 89%, 90%, 91%, 92%, 93%, 94%, 95%, 96%, 97%, 98%, 99%, or more) sequence identity thereto);
(e) optionally, a second promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto);
(f) a second 5′ flanking region located 5′ to a second passenger nucleotide sequence (e.g., any one of SEQ ID NOs: 752, 754, 756, 759, 762, 765, and 768 or a variant thereof with at least 85% (at least 86%, 87%, 88%, 89%, 90%, 91%, 92%, 93%, 94%, 95%, 96%, 97%, 98%, 99%, or more) sequence identity thereto);
(g) a second stem-loop sequence comprising, from 5′ to 3′:
   (i) a second 5′ stem-loop arm comprising the second passenger nucleotide sequence which is complementary or substantially complementary to a second guide sequence; (ii) a second loop region, wherein the second loop region comprises a second microRNA loop sequence (e.g., any one of SEQ ID NOs: 758, 761, 764, 767, and 770 or a variant thereof with one, two, or three nucleotide changes thereto);
   (iii) a second 3′ stem-loop arm comprising a second guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
and
(h) a second 3′ flanking region located 3′ to said second guide nucleotide sequence (e.g., any one of SEQ ID NOs: 753, 755, 757, 760, 763, 766, and 769).

In some embodiments, the expression cassette includes a polynucleotide having at least 85% (at least 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 785, 787, and 788.

In another aspect, the disclosure provides an expression cassette that includes a nucleotide sequence comprising, from 5′ to 3′:
(a) a first promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto);
(b) a first 5′ flanking region located 5′ to a first passenger nucleotide sequence (e.g., any one of SEQ ID NOs: 752, 754, 756, 759, 762, 765, and 768);
(c) a first stem-loop sequence comprising, from 5′ to 3′:
   (i) a first 5′ stem-loop arm comprising a first passenger nucleotide sequence which is complementary or substantially complementary to a first guide sequence;
   (ii) a first loop region, wherein the first loop region comprises a first microRNA loop sequence (e.g., any one of SEQ ID NOs: 758, 761, 764, 767, and 770);
   (iii) a first 3′ stem-loop arm comprising a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
   (d) a first 3′ flanking region located 3′ to said first guide nucleotide sequence (e.g., any one of SEQ ID NOs: 753, 755, 757, 760, 763, 766, and 769);
   (e) optionally, a second promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto);
   (f) a second 5′ flanking region located 5′ to a second guide nucleotide sequence (e.g., any one of SEQ ID NOs: 752, 754, 756, 759, 762, 765, and 768);
   (g) a second stem-loop sequence comprising, from 5′ to 3′:
   (i) a second 5′ stem-loop arm comprising a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
   (ii) a second loop region, wherein the second loop region comprises a second microRNA loop sequence (e.g., any one of SEQ ID NOs: 758, 761, 764, 767, and 770);
   (iii) a second 3′ stem-loop arm comprising a second passenger nucleotide seq uence which is complementary or substantially complementary to the second guide sequence;
and
(h) a second 3′ flanking region located 3′ to said second passenger nucleotide sequence (e.g., any one of SEQ ID NOs: 753, 755, 757, 760, 763, 766, and 769).

In another aspect, the disclosure provides an expression cassette that includes a nucleotide sequence comprising, from 5′ to 3′:
(a) a first promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto);
(b) a first 5′ flanking region located 5′ to a first guide nucleotide sequence (e.g., any one of SEQ ID NOs: 752, 754, 756, 759, 762, 765, and 768);
(c) a first stem-loop sequence comprising, from 5′ to 3′:
   (i) a first 5′ stem-loop arm comprising a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
   (ii) a first loop region, wherein the first loop region comprises a first microRNA loop sequence (e.g., any one of SEQ ID NOs: 758, 761, 764, 767, and 770);
   (iii) a first 3′ stem-loop arm comprising a first passenger nucleotide sequence which is complementary or substantially complementary to the first guide sequence;
(d) a first 3′ flanking region located 3′ to said first passenger nucleotide sequence (e.g., any one of SEQ ID NOs: 753, 755, 757, 760, 763, 766, and 769);
(e) optionally, a second promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto);
(f) a second 5′ flanking region located 5′ to a second passenger nucleotide sequence (e.g., any one of SEQ ID NOs: 752, 754, 756, 759, 762, 765, and 768);
(g) a second stem-loop sequence comprising, from 5′ to 3′:
   (i) a second 5′ stem-loop arm comprising a second passenger nucleotide sequence which is complementary or substantially complementary to a second guide sequence;
   (ii) a second loop region, wherein the second loop region comprises a second microRNA loop sequence (e.g., any one of SEQ ID NOs: 758, 761, 764, 767, and 770);
   (iii) a second 3′ stem-loop arm comprising a second guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
and
(h) a second 3′ flanking region located 3′ to said second guide nucleotide sequence (e.g., any one of SEQ ID NOs: 753, 755, 757, 760, 763, 766, and 769).

In some embodiments, the expression cassette includes a polynucleotide having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 786.

In another aspect, the disclosure provides an expression cassette that includes a nucleotide sequence comprising, from 5′ to 3′:
(a) a first promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto);
(b) a first 5′ flanking region located 5′ to a first guide nucleotide sequence (e.g., any one of SEQ ID NOs: 752, 754, 756, 759, 762, 765, and 768);
(c) a first stem-loop sequence comprising, from 5′ to 3′:
   (i) a first 5′ stem-loop arm comprising a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
   (ii) a first loop region, wherein the first loop region comprises a first microRNA loop sequence (e.g., any one of SEQ ID NOs: 758, 761, 764, 767, and 770);
   (iii) a first 3′ stem-loop arm comprising a first passenger nucleotide sequence which is complementary or substantially complementary to the first guide sequence;
(d) a first 3′ flanking region located 3′ to said first passenger nucleotide sequence (e.g., any one of SEQ ID NOs: 753, 755, 757, 760, 763, 766, and 769);
(e) optionally, a second promoter sequence (e.g., any one of the promoter sequences disclosed herein, such as those disclosed in, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto);
(f) a second 5′ flanking region located 5′ to a second guide nucleotide sequence (e.g., any one of SEQ ID NOs: 752, 754, 756, 759, 762, 765, and 768);
(g) a second stem-loop sequence comprising, from 5′ to 3′:
   (i) a second 5′ stem-loop arm comprising a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
   (ii) a second loop region, wherein the second loop region comprises a second microRNA loop sequence (e.g., any one of SEQ ID NOs: 758, 761, 764, 767, and 770);
   (iii) a second 3′ stem-loop arm comprising a second passenger nucleotide sequence which is complementary or substantially complementary to the second guide sequence;
and
(h) a second 3′ flanking region located 3′ to said second passenger nucleotide sequence (e.g., any one of SEQ ID NOs: 753, 755, 757, 760, 763, 766, and 769).

In some embodiments, the first promoter and/or, optionally, the second promoter is selected from the group consisting of a U6 promoter, H1 promoter, 7SK promoter, Apolipoprotein E-Human Alpha 1-Antitrypsin promoter, CAG promoter, CBA promoter, CK8 promoter, mU1a promoter, Elongation Factor 1a promoter, HSV promoter, Thyroxine Binding Globulin promoter, Synapsin promoter, RNA Binding Fox-1 Homolog 3 promoter, Calcium/Calmodulin Dependent Protein Kinase II promoter, neuron-specific enolase promoter, Platelet Derived Growth Factor Subunit β, Vesicular Glutamate Transporter promoter, Somatostatin promoter, Neuropeptide Y promoter, Vasoactive Intestinal Peptide promoter, Parvalbumin promoter, Glutamate Decarboxylase 65 promoter, Glutamate Decarboxylase 67 promoter, Dopamine Receptor D1 promoter, Dopamine Receptor D2 promoter, Complement C1q Like 2 promoter, Proopiomelanocortin promoter, Prospero Homeobox 1 promoter, Microtubule Associated Protein 1B promoter, and Tubulin Alpha 1 promoter.

In some embodiments, the first promoter is a SYN promoter (e.g., any one of SEQ ID NOs: 682-685 and 790 or a variant thereof having at least 85% (e.g., at least 86%, 90%, 95%, 96%, 97%, 98%, 99%, or more) sequence identity thereto and, optionally, the second promoter is a CAMKII promoter (e.g., any one of SEQ ID NOs: 687-691 and 802 or a variant thereof having at least 85% (e.g., at least 86%, 90%, 95%, 96%, 97%, 98%, 99%, or more) sequence identity thereto.

In some embodiments, the first 5′ flanking region and/or the second 5′ flanking region comprises a polynucleotide having at least 85% (e.g., at least 86%, 90%, 95%, 96%, 97%, 98%, 99%, or more) sequence identity to the nucleic acid sequence of any one of SEQ ID NOs: 752, 754, 756, 759, 762, 765, and 768. In some embodiments, the first 5′ flanking region and/or the second 5′ flanking region comprises a polynucleotide having the nucleic acid sequence of 752, 754, 756, 759, 762, 765, and 768.

In some embodiments, the first 3′ flanking region and/or the second 3′ flanking region comprises a polynucleotide having at least 85% (e.g., at least 86%, 90%, 95%, 96%, 97%, 98%, 99%, or more) sequence identity to the nucleic acid sequence of any one of SEQ ID NOs: 753, 755, 757, 760, 763, 766, and 769. In some embodiments, the first 3′ flanking region and/or the second 3′ flanking region comprises a polynucleotide having the nucleic acid sequence of any one of SEQ ID NOs: 753, 755, 757, 760, 763, 766, and 769.

In some embodiments, the first microRNA loop sequence and/or the second microRNA loop sequence is a miR-30, miR-155, miR-218-1, or miR-124-3 sequence. In some embodiments, the first microRNA loop sequence and/or the second microRNA loop sequence comprises a polynucleotide having at least 85% (e.g., at least 86%, 90%, 95%, 96%, 97%, 98%, 99%, or more) sequence identity to the nucleic acid sequence of any one of SEQ ID NOs: 758, 761, 764, 767, and 770. In some embodiments, the first microRNA loop sequence and/or the second microRNA loop sequence comprises a polynucleotide having the nucleic acid sequence of any one of SEQ ID NOs: 758, 761, 764, 767, and 770.

In some embodiments, the expression cassette comprises a 5′-inverted terminal repeat (ITR) sequence on the 5′ end of said expression cassette and a 3′-ITR sequence on the 3′ end of said expression cassette. In some embodiments, the 5′-ITR and 3′ ITR sequences are AAV2 5′-ITR and 3′ ITR sequences. In some embodiments, the 5′-ITR sequence comprises a polynucleotide having at least 85% (e.g., at least 86%, 90%, 95%, 96%, 97%, 98%, 99%, or more) sequence identity to the nucleic acid sequence of SEQ ID NO: 746 or SEQ ID NO: 747. In some embodiments, the 5′-ITR sequence comprises a polynucleotide having the nucleic acid sequence of SEQ ID NO: 746 or SEQ ID NO: 747. In some embodiments, the 3′-ITR sequence comprises a polynucleotide having at least 85% (e.g., at least 86%, 90%, 95%, 96%, 97%, 98%, 99%, or more) sequence identity to the nucleic acid sequence SEQ ID NO: 748, SEQ ID NO: 749, or SEQ ID NO: 789. In some embodiments, the 3′-ITR sequence comprises a polynucleotide having the nucleic acid sequence SEQ ID NO: 748, SEQ ID NO: 749, or SEQ ID NO: 789.

In some embodiments, the expression cassette further includes an enhancer sequence. In some embodiments, the enhancer sequence includes a polynucleotide having at least 85% (e.g., at least 86%, 90%, 95%, 96%, 97%, 98%, 99%, or more) sequence identity to the nucleic acid sequence of SEQ ID NO: 745. In some embodiments, the enhancer sequence includes a polynucleotide having the nucleic acid sequence of SEQ ID NO: 745.

In some embodiments, the expression cassette further includes an intron sequence. In some embodiments, the intron sequence comprises a polynucleotide having at least 85% (e.g., at least 86%, 90%, 95%, 96%, 97%, 98%, 99%, or more) sequence identity to the nucleic acid sequence of SEQ ID NO: 743 or SEQ ID NO: 744. In some embodiments, the intron sequence comprises a polynucleotide having the nucleic acid sequence of SEQ ID NO: 743 or SEQ ID NO: 744.

In some embodiments, the expression cassette further includes one or more polyadenylation signals. In some embodiments, the one or more polyadenylation signals is a rabbit beta-globin (RBG) polyadenylation signal. In some embodiments, the RBG polyadenylation signal comprises a polynucleotide having at least 85% (e.g., at least 86%, 90%, 95%, 96%, 97%, 98%, 99%, or more) sequence identity to the nucleic acid sequence of SEQ ID NO: 750, SEQ ID NO: 751, or SEQ ID NO: 792. In some embodiments, the RBG polyadenylation signal comprises a polynucleotide having the nucleic acid sequence of SEQ ID NO: 750, SEQ ID NO: 751, or SEQ ID NO: 792. In some embodiments, the polyadenylation signal is a bovine growth hormone (BGH) polyadenylation signal. In some embodiments, the BGH polyadenylation signal comprises a polynucleotide having at least 85% (e.g., at least 86%, 90%, 95%, 96%, 97%, 98%, 99%, or more) sequence identity to the nucleic acid sequence of SEQ ID NO: 793. In some embodiments, the BGH polyadenylation signal comprises a polynucleotide having the nucleic acid sequence of SEQ ID NO: 793.

In some embodiments, the expression cassette of the foregoing aspects and embodiments is incorporated into the vector of the foregoing aspect and embodiments.

In some embodiments, the expression cassette of the foregoing aspect does not include the sequence of any one of SEQ ID NOs: 772-774. In some embodiments, the expression cassette does not include the sequence of any one of SEQ ID NOs: 772-774 in combination with any one or more of the sequences of SEQ ID NOs: 1-771. In some embodiments, the expression cassette does not include the sequence of any one of SEQ ID NOs: 772-774 in combination with the sequence of SEQ ID NO: 68. In some embodiments, the expression cassette does not include the sequence of any one of SEQ ID NOs: 772-774 in combination with the sequence of SEQ ID NO: 68 and 649.

In some embodiments, the expression cassette further comprises one or more (e.g., 1, 2, or more) stuffer sequences. In some embodiments, the one or more stuffer sequences are positioned at the 3′ end of the expression cassette. In some embodiments, the one or more stuffer sequences have at least 85% (e.g., at least 86%, 90%, 95%, 96%, 97%, 98%, 99%, or more) sequence identity to the nucleic acid sequence of SEQ ID NO: 815. In some embodiments, the one or more stuffer sequences have at least 90% (e.g., at least 91%, 95%, 96%, 97%, 98%, 99%, or more) sequence identity to the nucleic acid sequence of SEQ ID NO: 815. In some embodiments, the one or more stuffer sequences have at least 95% (e.g., at least 96%, 97%, 98%, 99%, or more) sequence identity to the nucleic acid sequence of SEQ ID NO: 815. In some embodiments, the one or more stuffer sequences have at least 99% sequence identity to the nucleic acid sequence of SEQ ID NO: 815. In some embodiments, the one or more stuffer sequences have the nucleic acid sequence of SEQ ID NO: 815. In some embodiments, the one or more stuffer sequences have at least 85% (e.g., at least 86%, 90%, 95%, 96%, 97%, 98%, 99%, or more) sequence identity to the nucleic acid sequence of SEQ ID NO: 816. In some embodiments, the one or more stuffer sequences have at least 90% (e.g., at least 91%, 95%, 96%, 97%, 98%, 99%, or more) sequence identity to the nucleic acid sequence of SEQ ID NO: 816. In some embodiments, the one or more stuffer sequences have at least 95% (e.g., at least 96%, 97%, 98%, 99%, or more) sequence identity to the nucleic acid sequence of SEQ ID NO: 816. In some embodiments, the one or more stuffer sequences have at least 99% sequence identity to the nucleic acid sequence of SEQ ID NO: 816. In some embodiments, the one or more stuffer sequences have the nucleic acid sequence of SEQ ID NO: 816.

In another aspect, the disclosure provides a method of inhibiting Grik2 expression in a cell, the method including contacting the cell with at least one polynucleotide of the foregoing aspect and embodiments, the vector of the foregoing aspect and embodiments, or the expression cassette of the foregoing aspects and embodiments.

In some embodiments, the polynucleotide specifically hybridizes to a Grik2 mRNA and inhibits or reduces the expression of Grik2 in the cell. In some embodiments, the method reduces a level of Grik2 mRNA in the cell. In some embodiments, the method reduces a level of Grik2 mRNA in the cell by at least 10%, at least at least 15%, at least 20%, at least 25%, at least 30%, at least 35%, at least 40%, at least 45%, at least 50%, at least 55%, at least 60%, at least 65%, at least 70%, or at least 75% relative to a level of GluK2 protein in a cell treated with a control polynucleotide not capable of hybridizing to Grik2 mRNA or relative to a cell not treated with the polynucleotide.. In some embodiments, the method reduces a level of Gluk2 protein in the cell. In some embodiments, the method reduces a level of GluK2 protein in the cell by at least 10%, at least at least 15%, at least 20%, at least 25%, at least 30%, at least 35%, at least 40%, at least 45%, at least 50%, at least 55%, at least 60%, at least 65%, at least 70%, or at least 75% relative to a level of GluK2 protein in a cell treated with a control polynucleotide not capable of hybridizing to Grik2 mRNA or relative to a cell not treated with the polynucleotide.

In some embodiments, the cell is a neuron. In some embodiments, the neuron is a hippocampal neuron. In some embodiments, the hippocampal neuron is a DGC. In some embodiments, the DGC includes an aberrant recurrent mossy fiber axon. The cell may also be a neuronal cell derived from an induced pluripotent stem cell (iPSC), such as an iPSC-derived glutamatergic neuron that expresses Grik2.

In some embodiments, the method of the foregoing aspect does not include the use of a sequence of any one of SEQ ID NOs: 772-774. In some embodiments, the method does not include the use of the sequence of any one of SEQ ID NOs: 772-774 in combination with any one or more of the sequences of SEQ ID NOs: 1-771. In some embodiments, the method does not include the use of a sequence of any one of SEQ ID NOs: 772-774 in combination with the sequence of SEQ ID NO: 68. In some embodiments, the method does not include the use of a sequence of any one of SEQ ID NOs: 772-774 in combination with the sequence of SEQ ID NO: 68 and SEQ ID NO: 649.

In another aspect, the disclosure provides a method of treating or ameliorating a disorder in a subject in need thereof, the method including administering to the subject at least one polynucleotide of the foregoing aspect and embodiments, a vector of the foregoing aspect and embodiment, or an expression cassette of the foregoing aspects and embodiments (e.g., an expression cassette including a polynucleotide having at least 85% (e.g., at least 86%, 90%, 95%, 96%, 97%, 98%, 99%, or more) sequence identity to the nucleic acid sequence of any one of SEQ ID NOs: 775, 777, 779, 781, 783-788, 796, 798-801, 803, 805, 807, 809, 811, 813, 817, 819, and 821).

In some embodiments, the disorder is an epilepsy. In some embodiments, the epilepsy is a temporal lobe epilepsy (TLE), chronic epilepsy, and/or a refractory epilepsy. In some embodiments, the epilepsy is a TLE. In some embodiments, the TLE is a lateral TLE (ITLE). In some embodiments, the TLE is a mesial TLE (mTLE).

In one or more, or each of the embodiments described above, the subject is a human.

In some embodiments, the method of the foregoing aspect does not include administration of the sequence of any one of SEQ ID NOs: 772-774. In some embodiments, the method does not include administration of the sequence of any one of SEQ ID NOs: 772-774 in combination with any one or more of the sequences of SEQ ID NOs: 1-771. In some embodiments, the polynucleotide does not include administration of the sequence of any one of SEQ ID NOs: 772-774 in combination with the sequence of SEQ ID NO: 68. In some embodiments, the polynucleotide does not include administration of the sequence of any one of SEQ ID NOs: 772-774 in combination with the sequence of SEQ ID NO: 68 and SEQ ID NO: 649.

In another aspect, the disclosure provides a pharmaceutical composition including the polynucleotide of the foregoing aspect and embodiments, the vector of the foregoing aspect and embodiments, or the expression cassette of the foregoing aspects and embodiments, and a pharmaceutically acceptable carrier, diluent, or excipient.

In some embodiments, the pharmaceutical composition of the foregoing aspect does not include a polynucleotide with the sequence of any one of SEQ ID NOs: 772-774. In some embodiments, the pharmaceutical composition does not include a polynucleotide with the sequence of any one of SEQ ID NOs: 772-774 in combination with any one or more of the sequences of SEQ ID NOs: 1-771. In some embodiments, the pharmaceutical composition does not include a polynucleotide with the sequence of any one of SEQ ID NOs: 772-774 in combination with the sequence of SEQ ID NO: 68. In some embodiments, the pharmaceutical composition does not include a polynucleotide with the sequence of any one of SEQ ID NOs: 772-774 in combination with the sequence of SEQ ID NO: 68 and SEQ ID NO: 649.

In another aspect, the disclosure provides a kit including the pharmaceutical composition of the foregoing aspect and a package insert. In some embodiments, the package insert includes instructions for use of the pharmaceutical composition in the method of the foregoing aspects and embodiments.

In some embodiments, the kit of the foregoing aspect does not include a polynucleotide with the sequence of any one of SEQ ID NOs: 772-774. In some embodiments, the kit does not include a polynucleotide with the sequence of any one of SEQ ID NOs: 772-774 in combination with any one or more of the sequences of SEQ ID NOs: 1-771. In some embodiments, the kit does not include a polynucleotide with the sequence of any one of SEQ ID NOs: 772-774 in combination with the sequence of SEQ ID NO: 68. In some embodiments, the kit does not include a polynucleotide with the sequence of any one of SEQ ID NOs: 772-774 in combination with the sequence of SEQ ID NO: 68 and SEQ ID NO: 649.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments and are not intended to limit the claimed technology. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

In this application, unless otherwise clear from context, (i) the term “a” may be understood to mean “at least one”; (ii) the term “or” may be understood to mean “and/or”; and (iii) the terms “including” and “comprising” may be understood to encompass itemized components or steps whether presented by themselves or together with one or more additional components or steps.

The term “about” refers to an amount that is ±10% of the recited value and may be ±5% of the recited value or ±2% of the recited value.

The terms “3′ untranslated region” and “3′ UTR” refer to the region 3′ with respect to the stop codon of an mRNA molecule (e.g., a Grik2 mRNA). The 3′ UTR is not translated into protein, but includes regulatory sequences important for polyadenylation, localization, stabilization, and/or translation efficiency of an mRNA transcript. Regulatory sequences in the 3′ UTR may include enhancers, silencers, AU-rich elements, poly-A tails, terminators, and microRNA recognition sequences. The terms “3′ untranslated region” and “3′ UTR” may also refer to the corresponding regions of the gene encoding the mRNA molecule.

The term “5′ untranslated region” and “5′ UTR” refer to a region of an mRNA molecule (e.g., a Grik2 mRNA) that is 5′ with respect to the start codon. This region is important for the regulation of translation initiation. The 5′ UTR can be entirely untranslated or may have some of its regions translated in some organisms. The transcription start site marks the start of the 5′ UTR and ends one nucleotide before the start codon. In eukaryotes, the 5′ UTR includes a Kozak consensus sequence harboring the start codon. The 5′ UTR may include cis-acting regulatory elements also known as upstream open reading frames that are important for the regulation of translation. This region may also harbor upstream AUG codons and termination codons. Given its high GC content, the 5′ UTR may form secondary structures, such as hairpin loops that play a role in the regulation of translation. The term “administration” refers to providing or giving a subject a therapeutic agent (e.g., an antisense oligonucleotide (ASO) that binds to and inhibits the expression of a Grik2 mRNA, or a vector encoding the same, as is disclosed herein), by any effective route. Exemplary routes of administration are described herein and below (e.g. intracerebroventricular injection, intrathecal injection, intraparenchymal injection, intravenous injection, and stereotactic injection).

The term “adeno-associated viral vector” or “AAV vector” refers to a vector derived from an adeno-associated virus serotype, including without limitation, AAV1, AAV2, AAV3, AAV4, AAVS, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAVIK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10 , AAV.HSC11, AAV.HSC12, AAV.HSC13, AAV.HSC14, AAV.HSC15, AAV-TT, AAV-DJ8, or AAV.HSC16. AAV vectors can have one or more of the AAV wild-type genes deleted in whole or part, e.g., the rep and/or cap genes, but retain functional flanking ITR sequences. Functional ITR sequences promote the rescue, replication, and packaging of the AAV virion. Thus, an AAV vector is defined herein to include at least those sequences required in cis for replication and packaging (e.g., functional ITRs) of the virus. ITRs do not need to be the wild-type polynucleotide sequences and may be altered, e.g., by the insertion, deletion, or substitution of nucleotides, so long as the sequences provide for functional rescue, replication, and packaging. AAV expression vectors are constructed using known techniques to at least provide as operatively linked components in the direction of transcription, control elements including a transcriptional initiation region, the DNA of interest (e.g., a polynucleotide encoding an ASO agent of the disclosure) and a transcriptional termination region.

The terms “adeno-associated virus inverted terminal repeats” and “AAV ITRs” refer to art-recognized regions flanking each end of the AAV genome which function together in cis as origins of DNA replication and as packaging signals for the virus. AAV ITRs, together with the AAV rep coding region, provide for the efficient excision and integration of a polynucleotide sequence interposed between two flanking ITRs into a mammalian genome. The polynucleotide sequences of AAV ITR regions are known. As used herein, an “AAV ITR” does not necessarily include the wild-type polynucleotide sequence, which may be altered, e.g., by the insertion, deletion or substitution of nucleotides. Additionally, the AAV ITR may be derived from any of several AAV serotypes, including without limitation AAV1, AAV2, AAV3, AAV4, AAVS, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAVIK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10 , AAV.HSC11, AAV.HSC12, AAV.HSC13, AAV.HSC14, AAV.HSC15, AAV-TT, AAV-DJ8, or AAV.HSC16, among others. Furthermore, 5′ and 3′ ITRs which flank a selected polynucleotide sequence in an AAV vector need not be identical or derived from the same AAV serotype or isolate, so long as they function as intended, e.g., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV1, AAV2, AAV3, AAV4, AAVS, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAVIK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16, among others.

The terms “antisense oligonucleotide” and “ASO” refer to an oligonucleotide capable of hybridizing through complementary base-pairing with a target mRNA molecule (e.g., a Grik2 mRNA) and inhibiting its expression through mRNA destabilization and degradation, or inhibition of translation. Non-limiting examples of ASOs include short interfering RNAs (siRNAs), short hairpin RNAs (shRNAs), and microRNAs (miRNAs).

The term “cDNA” refers to a nucleic acid sequence that is a DNA equivalent of an mRNA sequence (i.e., having uridine substituted with thymidine). Generally, the terms cDNA and mRNA may be used interchangeably in reference to a particular gene (e.g., Grik2 gene) as one of skill in the art would understand that a cDNA sequence is the same as the mRNA sequence with the exception that uridines are read as thymidines.

The term “coding sequence” corresponds to a nucleic acid sequence of an mRNA molecule that encodes a protein or a portion thereof. Relatedly, a “non-coding sequence” corresponds to a nucleic acid sequence of an mRNA molecule that does not encode a protein or a portion thereof. Non-limiting examples of non-coding sequences include 5′ and 3′ untranslated regions (UTRs), introns, polyA tail, promoters, enhancers, terminators, and other cis-regulatory sequences.

The term “complementary,” when used to describe a first nucleotide or nucleoside sequence in relation to a second nucleotide or nucleoside sequence, refers to the ability of an oligonucleotide or polynucleotide including the first nucleotide sequence to hybridize and form a duplex structure under certain conditions with an oligonucleotide or polynucleotide including the second nucleotide sequence. Such conditions can, for example, be stringent conditions, where stringent conditions can include: 400 mM NaCl, mM PIPES pH 6.4, 1 mM EDTA, 50° C., or 70° C., for 12-16 hours followed by washing (see, e.g., “Molecular Cloning: A Laboratory Manual”, Sambrook, et al. (1989) Cold Spring Harbor Laboratory Press). Other conditions, such as physiologically relevant conditions as can be encountered inside an organism, can apply. Methods of determining the set of conditions most appropriate for a test of complementarity of two sequences in accordance with the ultimate application of the hybridized nucleotides or nucleosides are well-known in the art.

“Complementary” sequences, as used herein, can also include, or be formed entirely from, non-Watson-Crick base pairs and/or base pairs formed from non-natural and alternative nucleotides, in so far as the above requirements with respect to their ability to hybridize are fulfilled. Such non-Watson-Crick base pairs include, but are not limited to, G:U Wobble or Hoogstein base pairing. Complementary sequences between an oligonucleotide and a target sequence as described herein, include base-pairing of the oligonucleotide or polynucleotide including a first nucleotide sequence to an oligonucleotide or polynucleotide including a second nucleotide sequence over the entire length of one or both nucleotide sequences. Such sequences can be referred to as “fully complementary” with respect to each other herein. Where a first sequence is referred to as “substantially complementary” with respect to a second sequence herein, the two sequences can be fully complementary or they can form one or more, but generally no more than 10, 9, 8, 7, 6, 5, 4, 3, 2, or 1 mismatched base pairs upon hybridization for a duplex of up to base pairs, while retaining the ability to hybridize under the conditions most relevant to their ultimate application, e.g., binding to and inhibiting the expression of an mRNA, such as a Grik2 mRNA. For example, a polynucleotide is complementary to at least a part of the mRNA of interest if the sequence is substantially complementary to a non-interrupted portion of the mRNA of interest.

The term “region of complementarity” refers to the region on the oligonucleotide that is substantially complementary to all or a portion of a gene, primary transcript, a sequence (e.g., a target sequence), or processed mRNA, so as to interfere with expression of the endogenous gene (e.g., Grik2). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the oligonucleotide.

The terms “conservative amino acid substitution”, “conservative substitution,” and “conservative mutation,” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally
occurring amino acids

| | | | | Electrostatic | |
|---|---|---|---|---|---|
| | | | Side- | character at | |
| | 3 Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium at | |
| | | | | pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

The phrase “contacting a cell with an oligonucleotide,” such as an oligonucleotide disclosed herein, includes contacting a cell by any possible means. Contacting a cell with an oligonucleotide includes contacting a cell in vitro with the oligonucleotide or contacting a cell in vivo with the oligonucleotide. Contacting a cell with a polynucleotide may also refer to contacting the cell with a nucleic acid vector encoding the polynucleotide or a pharmaceutical composition containing the same. The contacting may be done directly or indirectly. Thus, for example, the oligonucleotide may be put into physical contact with the cell by the individual performing the method, or alternatively, the oligonucleotide agent may be put into a situation that will permit or cause it to subsequently come into contact with the cell. Contacting a cell in vitro may be done, for example, by incubating the cell with the oligonucleotide. Contacting a cell in vivo may be done, for example, by injecting the oligonucleotide into or near the tissue where the cell is located, or by injecting the oligonucleotide agent into another area, e.g., the bloodstream or the subcutaneous space, such that the agent will subsequently reach the tissue where the cell to be contacted is located. Combinations of in vitro and in vivo methods of contacting are also possible. For example, a cell may also be contacted in vitro with an oligonucleotide and subsequently transplanted into a subject.

Contacting a cell with an oligonucleotide includes “introducing” or “delivering the oligonucleotide into the cell” by facilitating or effecting uptake or absorption into the cell. Absorption or uptake of an oligonucleotide can occur through unaided diffusive or active cellular processes, or by auxiliary agents or devices. Introducing an oligonucleotide into a cell may be in vitro and/or in vivo. For example, for in vivo introduction, oligonucleotide(s) can be injected into a tissue site or administered systemically. In vitro introduction into a cell includes methods known in the art such as electroporation and lipofection. In another example, an oligonucleotide can be introduced into a cell by transduction, such as by way of a viral vector encoding the polynucleotide. The viral vector may undergo cellular processing (e.g., cellular internalization, capsid shedding, transcription of the polynucleotide, and processing by Drosha and Dicer) in order to express the encoded polynucleotide. Further approaches are described herein below and/or are known in the art.

The terms “disrupt expression of,” “inhibit expression of,” or “reduce the expression of,” with respect to a gene (e.g., Grik2), refers to preventing or reducing the formation of a functional gene product (e.g., a GluK2 protein). A gene product is functional if it fulfills its normal (wild-type) function(s). Disruption of the expression of a gene prevents or reduces the expression of a functional protein encoded by the gene. Gene expression may be disrupted by using, e.g., an interfering RNA molecule (e.g., an ASO), such as those described herein.

The terms “effective amount,” “therapeutically effective amount,” and a “sufficient amount” of composition, vector construct, or viral vector described herein refer to a quantity sufficient to, when administered to the subject, including a mammal, for example a human, effect beneficial or desired results, including clinical results. As such, an “effective amount” or synonym thereof depends upon the context in which it is being applied. For example, in the context of treating temporal lobe epilepsy (TLE), it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder and its severity, the identity of the subject (e.g., age, sex, weight), host being treated, and/or, in the case of an epilepsy, the size (e.g., brain volume) of the epileptic focus, and the like, but can nevertheless be determined according to methods well-known in the art. Also, as used herein, a “therapeutically effective amount” of a composition, vector construct, or viral vector of the disclosure is an amount which results in a beneficial or desired result in a subject as compared to a control. As defined herein, a therapeutically effective amount of a composition, vector construct, viral vector, or cell of the disclosure may be readily determined by methods known in the art, such as those methods described herein. A dosage regime may be adjusted to provide a suitable endpoint therapeutic response (e.g., a statistically significant reduction in the occurrence of epileptic seizure in a treated subject).

The term “epilepsy” refers to one or more neurological disorders that clinically present with recurrent epileptic seizures. Epilepsy can be classified according the electroclinical syndromes following the Classification and Terminology of the International League Against Epilepsy (ILAE; Berg et al., 2010). These syndromes can be categorized by age at onset, distinctive constellations (surgical syndromes), and structural-metabolic causes, such as: (A) age at onset: (i) neonatal period includes benign familial neonatal epilepsy (BFNE), early myoclonic encephalopathy (EME), Ohtahara syndrome; (ii) infancy period includes epilepsy of infancy with migrating focal seizures, West syndrome, myoclonic epilepsy in infancy (MEI), benign infantile epilepsy, benign familial infantile epilepsy, Dravet syndrome, myoclonic encephalopathy in nonprogressive disorders; (iii) childhood period includes febrile seizures plus (FS+), Panayiotopoulos syndrome, epilepsy with myoclonic atonic (previously astatic) seizures, benign epilepsy with centrotemporal spikes (BECTS), autosomal-dominant nocturnal frontal lobe epilepsy (ADNFLE), late onset childhood occipital epilepsy (Gastaut type), epilepsy with myoclonic absences, Lennox-Gastaut syndrome, epileptic encephalopathy with continuous spike-and-wave during sleep (CSWS), Landau-Kleffner syndrome (LKS), childhood absence epilepsy (CAE); (iv) adolescence—adult period includes juvenile absence epilepsy (JAE) juvenile myoclonic epilepsy (JME), epilepsy with generalized tonic—clonic seizures alone, progressive myoclonus epilepsies (PME), autosomal dominant epilepsy with auditory features (ADEAF), other familial temporal lobe epilepsies; (v) variable age onset includes familial focal epilepsy with variable foci (childhood to adult), reflex epilepsies; (B) distinctive constellations (surgical syndromes) include mesial temporal lobe epilepsy (MTLE), Rasmussen syndrome, gelastic seizures with hypothalamic hamartoma, hemiconvulsion—hemiplegia—epilepsy; (C) epilepsies attributed to and organized by structural-metabolic causes include malformations of cortical development (hemimegalencephaly, heterotopias, etc.), neurocutaneous syndromes (tuberous sclerosis complex and Sturge-Weber), tumor, infection, trauma, angioma, perinatal insults, and stroke. The term “refractory epilepsy” refers to an epilepsy which is refractory to pharmaceutical treatment; that is to say that current pharmaceutical treatment does not allow an effective treatment of patients' disease (see for example Englot et al. J Neurosurg Pediatr 12:134-41 (2013)).

The term “exon” refers to a region within the coding region of a gene (e.g., a Grik2 gene), the nucleotide sequence of which determines the amino acid sequence of the corresponding protein. The term “exon” also refers to the corresponding region of the RNA transcribed from a gene. Exons are transcribed into pre-mRNA and may be included in the mature mRNA depending on the alternative splicing of the gene. Exons that are included in the mature mRNA following processing are translated into protein. The sequence of the exon determines the amino acid composition of the protein. Alternatively, exons that are included in the mature mRNA may be non-coding (e.g., exons that do not translate into protein).

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include mRNAs, which are modified by processes such as capping, polyadenylation, methylation, and editing, and proteins (e.g., GluK2) modified by, for example, methylation, acetylation, phosphorylation, ubiquitination, SUMOylation, ADP-ribosylation, myristoylation, and glycosylation.

The term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: a decrease or increase in the quantity or concentration of mRNA encoding a corresponding protein (as assessed, e.g., using RNA detection procedures described herein or known in the art, such as quantitative polymerase chain reaction (qPCR) and RNA seq techniques), a decrease or increase in the quantity or concentration of a corresponding protein (as assessed, e.g., using protein detection methods described herein or known in the art, such as enzyme-linked immunosorbent assays (ELISA), among others), and/or a decrease or increase in the activity of a corresponding protein (e.g., in the case of an ion channel, as assessed using electrophysiological methods described herein or known in the art) in a sample obtained from the subject.

The term “GluK2”, also known as “GluR6”, “GRIK2”, “MRT6”, “EAA4”, or “GluK6”, refers to the glutamate ionotropic receptor kainate type subunit 2 protein, as named in the currently used IUPHAR nomenclature (Collingridge, G.L., Olsen, R.W., Peters, J., Spedding, M., 2009. A nomenclature for ligand-gated ion channels. Neuropharmacology 56, 2-5). The terms “GluK2-containing KAR,” “GluK2 receptor,” “GluK2 protein,” and “GluK2 subunit” may be used interchangeably throughout and generally refer to the protein encoded by or expressed by a Grik2 gene.

The terms, “guide strand,” or “guide sequence” refer to a component of a stem-loop RNA structure (e.g., an shRNA or microRNA) positioned on either the 5′ or the 3′ stem-loop arm of the stem-loop structure, wherein the guide strand/sequence includes a Grik2 mRNA antisense sequence (e.g., any one of SEQ ID NOs: 1-100 or a variant thereof having at least 85% (e.g., at least 86%, 90%, 95%, 96%, 97%, 98%, 99%, or more) sequence identity to the nucleic acid sequence of any one of SEQ ID NOs: 1-100) capable of binding to and inhibiting the expression of the Grik2 mRNA. The guide strand/sequence may also include additional sequences, such as, e.g., spacer or linker sequences. The guide sequence may be complementary or substantially complementary (e.g., having no more than 5, 4, 3, 2, or 1 mismatches) to a passenger strand/sequence of the stem-loop RNA structure.

The term “ionotropic glutamate receptors” include members of the NMDA (N-methyl-D-aspartate), AM PA (α-amino-3-hydroxy-5-methyl-4-isoxazoleproprionic acid) and kainate receptor (KAR) classes. Functional KARs can be assembled into tetrameric assemblies from the homomeric or heteromeric combination of five subunits named GluK1, GluK2, GluK3, GluK4 and GluKsubunits (Reiner et al., 2012). The targets of the disclosure are, in some instances, KAR complexes composed of GluK2 and GluK5. Inhibiting the expression of Grik2 gene is sufficient to abolish GluK2/GluKkainate receptor function, given the observation that the GluKsubunit by itself does not form functional homomeric channels.

An “inhibitor of expression” refers to an agent (e.g., an ASO agent of the disclosure) that has a biological effect to inhibit or decrease the expression of a gene, e.g., the Grik2 gene. Inhibiting expression of a gene, e.g., the Grik2 gene, will typically result in a decrease or even abolition of the gene product (protein, e.g., GluK2 protein) in target cells or tissues, although various levels of inhibition may be achieved. Inhibiting or decreasing expression is typically referred to as knockdown.

The term “isolated polynucleotide” refers to an isolated molecule including two or more covalently linked nucleotides. Such covalently linked nucleotides may also be referred to as nucleic acid molecules. Generally, an “isolated” polynucleotide refers to a polynucleotide that is man-made, chemically synthesized, purified, and/or heterologous with respect to the nucleic acid sequence from which it is obtained.

The term “microRNA” refers to a short (e.g., typically ˜22 nucleotide) sequence of non-coding RNA that regulates mRNA translation and thus influences target protein abundance. Some microRNAs are transcribed from a single, monocistronic gene, while others are transcribed as part of multigene gene clusters. The structure of a microRNA may include 5′ and 3′ flanking sequences, hairpin sequences including stem and stem loop sequences. During processing within the cell, an immature microRNA is truncated by Drosha, which cleaves off the 5′ and 3′ flanking sequences. Subsequently, the microRNA molecule is translocated from the nucleus to the cytoplasm, where it undergoes cleavage of the loop region by Dicer. The biological action of microRNAs is exerted at the level of translational regulation through binding to regions of the mRNA molecule, typically the 3′ untranslated region, and leading to the cleavage, degradation, destabilization, and/or less efficient translation of the mRNA. Binding of the microRNA to its target is generally mediated by a short (e.g., 6-8 nucleotide) “seed region” within the hairpin sequence of the microRNA. Throughout the disclosure, the term siRNA may include its equivalent miRNA, such that the miRNA encompasses the same bases that have homology to the target (e.g., in the seed region) as its equivalent siRNA. As described herein, a microRNA may be a non-naturally occurring microRNA, such as a microRNA having one or more heterologous nucleic acid sequences.

The term “nucleotide” is defined as a modified or naturally occurring deoxyribonucleotide or ribonucleotide. Nucleotides typically include purines and pyrimidines, which include thymidine, cytidine, guanosine, adenosine and uridine. The terms “oligonucleotide” as used herein is defined as an oligomer of the nucleotides defined above or modified nucleotides disclosed herein. The term “oligonucleotide” refers to a nucleic acid sequence, 3′-5′ or 5′-3′ oriented, which may be single- or double-stranded. The oligonucleotide used in the context of the disclosure may, in particular, be DNA or RNA. The term also includes “oligonucleotide analog” which refers to an oligonucleotide having (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide e.g., single-stranded RNA or single-stranded DNA). Particularly, analogs are those having a substantially uncharged, phosphorus containing backbone. A substantially uncharged, phosphorus containing backbone in an oligonucleotide analog is one in which a majority of the subunit linkages, e.g., between 50-100%, typically at least 60% to 100% or 75% or 80% of its linkages, are uncharged, and contain a single phosphorous atom. Furthermore, the term “oligonucleotide” refers to an oligonucleotide sequence that is inverted relative to its normal orientation for transcription and so corresponds to an RNA or DNA sequence that is complementary to a target gene mRNA molecule expressed within the host cell. An antisense guide strand may be constructed in a number of different ways, provided that it is capable of interfering with the expression of a target gene. For example, the antisense guide strand can be constructed by reverse-complementing the coding region (or a portion thereof) of the target gene relative to its normal orientation for transcription to allow the transcription of its complement, (e.g., RNAs encoded by the antisense and sense gene may be complementary). The oligonucleotide need not have the same intron or exon pattern as the target gene, and noncoding segments of the target gene may be equally effective in achieving antisense suppression of target gene expression as coding segments such as an ASO. In some cases, the ASO has the same exon pattern as the target gene.

The oligonucleotide may be of any length that permits targeting and hybridization to a Grik2 mRNA (e.g., the oligonucleotide is perfectly, or substantially complementary to at least a region of a Grik2 mRNA), and may range from about 10-50 base pairs in length, e.g., about 15-50 base pairs in length or about 18-50 base pairs in length, for example, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the disclosure.

The terms “passenger strand” and “passenger sequence” refer to a component of a stem-loop RNA structure (e.g., an shRNA or microRNA) positioned on either the 5′ or the 3′ stem-loop arm of the stem-loop structure that includes a sequence complementary or substantially complementary (e.g., having no more than 5, 4, 3, 2, or 1 mismatches to Grik2 mRNA antisense sequence (e.g., any one of SEQ ID NOs: 1-100 or a variant thereof having at least 85% (e.g., at least 86%, 90%, 95%, 96%, 97%, 25 98%, 99%, or more) sequence identity to the nucleic acid sequence of any one of SEQ ID NO: 1-108). The passenger strand/sequence may also include additional sequences, such as, e.g., spacer or linker sequences. The passenger sequence may be complementary or substantially complementary to a guide strand/sequence of the stem-loop RNA structure.

The term “plasmid” refers to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids, which have a bacterial origin of replication, and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

The term “positional entropy,” as it applies to an individual nucleotide within a polynucleotide (e.g., a Grik2 mRNA), refers to thermodynamic quantity that represents the number of molecular positions, configurations, or arrangements that the nucleotide can assume given the constraints and local topology imposed by the mRNA secondary structure. Low positional entropy at a specific nucleotide position indicates that the nucleotide can occupy a low number of positional configurations. High positional entropy at a specific nucleotide position indicates that the nucleotide can occupy a high number of positional configurations. Nucleotides within a polynucleotide chain may exhibit low positional entropy as a result of being involved in base-pairing with another nucleotide, thereby constraining the total number of positional configurations that the base-paired nucleotide can assume. Inversely, nucleotides within a polynucleotide may exhibit high positional entropy as a result of being unhybridized, thereby having more degrees of freedom with respect to its positional configuration relative to a base-paired nucleotide. The term “average positional entropy” refers to a mean value of the positional entropy values across all nucleotide positions of a given sequence. For example, average positional entropy can be calculated over at least 2 (e.g., at least 2, 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, 1000, or more) nucleotides. In a particular example, the average positional entropy is calculated over 2 or more nucleotides. In another example, the average positional entropy is calculated over or more nucleotides. In another example, the average positional entropy is calculated over or more nucleotides. In another example, the average positional entropy is calculated over or more nucleotides. In another example, the average positional entropy is calculated over or more nucleotides. In another example, the average positional entropy is calculated over or more nucleotides. In another example, the average positional entropy is calculated over or more nucleotides. In another example, the average positional entropy is calculated over or more nucleotides. In another example, the average positional entropy is calculated over or more nucleotides. In another example, the average positional entropy is calculated over 45 or more nucleotides. In another example, the average positional entropy is calculated over 50 or more nucleotides. In another example, the average positional entropy is calculated over 55 or more nucleotides. In another example, the average positional entropy is calculated over 60 or more nucleotides. In another example, the average positional entropy is calculated over 65 or more nucleotides. In another example, the average positional entropy is calculated over 70 or more nucleotides. In another example, the average positional entropy is calculated over 75 or more nucleotides. In another example, the average positional entropy is calculated over 80 or more nucleotides. In another example, the average positional entropy is calculated over 85 or more nucleotides. In another example, the average positional entropy is calculated over 90 or more nucleotides. In another example, the average positional entropy is calculated over 95 or more nucleotides. In another example, the average positional entropy is calculated over 100 or more nucleotides.

Methods of quantifying positional entropy of a nucleotide within a polynucleotide sequence are well-known in the art. The secondary structures of single-stranded polynucleotides, such as mRNA or RNA inhibitors that have a high positional entropy (closer to zero; in kcal/mol) as predicted in a folding algorithm (such as RNAfold), have low likelihood of forming strong, stable duplexes within its own structure, such as stem-loops. This predicted high positional entropy, single-stranded RNA typically exhibits high affinity for its binding target (see, e.g. PCT International Publication No. WO 2015/073360, published on 21 May 2015). Unpaired regions (unpaired loops and unpaired stems) of Grik2 mRNA are predicted to have high positional entropy (values closer to zero; in kcal/mol) and are favorable for interaction with guide sequences.

The term “promoter” refers to a recognition site on DNA that is bound by an RNA polymerase.

The polymerase drives transcription of the polynucleotide. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of polynucleotides, vectors, and target cell types.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are well-known in the art, for example, using publicly available computer software such as BLAST, BLAST-2, or Megalign software. Using well-recognized and conventional methods, the appropriate parameters can be determined for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

The term “pharmaceutical composition,” as used herein, represents a composition containing a compound (e.g., an ASO or vector containing the same) described herein formulated with a pharmaceutically acceptable excipient, and in some instances may be manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup), topical administration (e.g., as a cream, gel, lotion, or ointment), intravenous administration (e.g., as a sterile solution free of particulate emboli and in a solvent system suitable for intravenous use), intrathecal injection, intracerebroventricular injections, intraparenchymal injection, or in any other pharmaceutically acceptable formulation.

A “pharmaceutically acceptable excipient,” refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The compounds (e.g., ASOs and vectors containing the same) described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “regulatory sequence” includes promoters, enhancers and other expression control elements (e.g., polyadenylation signals) that control the transcription or translation of a gene. Such regulatory sequences are described, for example, in Perdew et al., Regulation of Gene Expression (Humana Press, New York, NY, (2014)); incorporated herein by reference.

The terms “target” or “targeting” refers to the ability of an ASO agent (e.g., such as an ASO agent described herein) to specifically bind through complementary base pairing to a Grik2 gene or mRNA encoding a GluK2 protein.

The term “single-stranded region” corresponds to a region of a predicted secondary structure of a Grik2 mRNA (e.g., Grik2 mRNA having the nucleic acid sequence of SEQ ID NO: 115 or a variant thereof having at least 85% (e.g., at least 86%, 90%, 95%, 96%, 97%, 98%, 99%, or more) sequence identity to the nucleic acid sequence of SEQ ID NO: 115) that is single-stranded (e.g., unhybridized to other nucleotides within the mRNA) or substantially single-stranded (e.g., having no more than 5% of the nucleotides within the region hybridized to other nucleotides of the same Grik2 mRNA molecule). Non-limiting examples of single-stranded regions of a Grik2 mRNA included predicted loop regions 1-14 (SEQ ID NOs: 145-158) and predicted unpaired regions 1-(SEQ ID NOs: 159-163) of the Grik2 mRNA (SEQ ID NO: 115).

The terms “short interfering RNA” and “siRNA” refer to a double stranded nucleic acid in which each strand comprises RNA, RNA analog(s) or RNA and DNA. The siRNA molecule can include between 19 and 23 nucleotides (e.g., 21 nucleotides). The siRNA typically has 2 bp overhangs on the 3′ ends of each strand such that the duplex region in the siRNA comprises 17-21 nucleotides (e.g., 19 nucleotides). Typically, the antisense strand of the siRNA is sufficiently complementary with the target sequence of the target gene/RNA. siRNA molecules operate within the RNA interference pathway, leading to inhibition of mRNA expression by binding to a target mRNA (e.g., Grik2 mRNA) and degrading the mRNA through Dicer-mediated mRNA cleavage. Throughout the disclosure, the term siRNA is meant to include its equivalent miRNA, such that the miRNA encompasses the same bases that have homology to the target as its equivalent siRNA.

The terms “short hairpin RNA” and “shRNA” refer to a single-stranded RNA of 50 to 100 nucleotides that forms a stem-loop structure in a cell, which contains a loop region of to nucleotides, and long complementary RNAs of to 50 nucleotides at both sides of the loop region, which form a double-stranded stem by base pairing between the complementary RNA sequences; and, in some cases, an additional 1 to 500 nucleotides included before and after each complementary strand forming the stem. For example, shRNA generally requires specific sequences 3′ of the hairpin to terminate transcription by RNA polymerase. Such shRNAs generally bypass processing by Drosha due to their inclusion of short 5′ and 3′ flanking sequences. Other shRNAs, such as “shRNA-like microRNAs,” which are transcribed from RNA polymerase II, include longer 5′ and 3′ flanking sequences, and require processing in the nucleus by Drosha, after which the cleaved shRNA is exported from the nucleus to cytosol and further cleaved in the cytosol by Dicer. Like siRNA, shRNA binds to the target mRNA in a sequence specific manner, thereby cleaving and destroying the target mRNA, and thus suppressing expression of the target mRNA.

The terms “subject” and “patient” refer to an animal (e.g., a mammal, such as a human). A subject to be treated according to the methods described herein may be one who has been diagnosed with an epilepsy (e.g., TLE), or one at risk of developing this condition. Diagnosis may be performed by any method or technique known in the art. A subject to be treated according to the present disclosure may have been subjected to standard tests or may have been identified, without examination, as one at risk due to the presence of one or more risk factors associated with the disease or condition.

The terms “temporal lobe epilepsy” or “TLE” refers to a chronic neurological condition characterized by chronic and recurrent seizures (epilepsy) which originate in the temporal lobe of the brain. This disease is different from acute seizures in naïve brain tissue since TLE is characterized by morpho-functional reorganization of neuronal networks and sprouting of recurrent mossy fibers from granule cells of the dentate gyrus of the hippocampus, whereas acute seizures in naïve tissue do not precipitate such circuit-specific reorganization. TLE may result from an emergence of an epileptogenic focus in one or both hemispheres of the brain.

The terms “transduction” and “transduce” refer to a method of introducing a nucleic acid material (e.g., a vector, such as a viral vector construct, or a part thereof) into a cell and subsequent expression of a polynucleotide encoded by the nucleic acid material (e.g., the vector construct or part thereof) in the cell.

The term “treatment” or “treat” refers to both prophylactic and preventive treatment as well as curative or disease modifying treatment, including treatment of a patient at risk of contracting the disease or suspected to have contracted the disease, as well as a patient who is ill or has been diagnosed as suffering from a disease or medical condition. Treatment also includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria (e.g., disease manifestation).

The term “vector” includes a nucleic acid vector, e.g., a DNA vector, such as a plasmid, an RNA vector, or another suitable replicon (e.g., viral vector). A variety of vectors have been developed for the delivery of polynucleotides encoding exogenous polynucleotides or proteins into a prokaryotic or eukaryotic cell. Examples of such expression vectors are disclosed in, e.g., WO 1994/011026; incorporated herein by reference as it pertains to vectors suitable for the expression of a nucleic acid material of interest. Expression vectors suitable for use with the compositions and methods described herein contain a polynucleotide sequence as well as, e.g., additional sequence elements used for the expression of heterologous nucleic acid materials (e.g., an ASO) in a mammalian cell. Certain vectors that can be used for the expression of the ASO agents described herein include plasmids that contain regulatory sequences, such as promoter and enhancer regions, which direct gene transcription. Other useful vectors for expression of ASO agents disclosed herein contain polynucleotide sequences that enhance the rate of translation of these polynucleotides or improve the stability or nuclear export of the RNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

The term “Total Free Energy of Binding” refers to a thermodynamic property of a nucleotide or polynucleotide (measured in kcal/mol) that corresponds to the free energy of the process of a nucleotide or polynucleotide (e.g., an ASO agent of the disclosure) hybridizing to its corresponding target sequence on the Grik2 mRNA (e.g., SEQ ID NO: 115), including opening the target region on the mRNA, generation of single-stranded guide, and hybridization of the single-stranded siRNA guide to its single-stranded mRNA target sequence. In the context of the present disclosure, more negative values of the Total Free Energy of Binding for a particular ASO sequence are generally associated with reduced efficacy of knockdown of Grik2 mRNA expression by said ASO, whereas values closer to zero generally reflect an increased knockdown efficacy.

The term “Energy from Duplex Formation” refers to a thermodynamic property of a nucleotide or polynucleotide (measured in kcal/mol) that corresponds to the free energy of hybridization of a single-stranded siRNA guide to a single-stranded mRNA sequence (e.g., Grik2 mRNA). In the context of the present disclosure, more negative Energy of Duplex Formation values for a given nucleotide or polynucleotide reflect that formation of a duplex is more favorable than the formation of a duplex for which the Energy of Duplex Formation is closer to zero, and also reflect a reduced knockdown efficacy of Grik2 mRNA expression. Therefore, this value indicates an inverse relationship between the favorability of duplex formation and knockdown efficacy, suggesting that energy of duplex formation provides a stronger measure for determining the favorability of duplex separation (its inverse) rather than duplex formation. Thus, the more negative a value of Energy from Duplex Formation, the more stable the duplex. It follows that a less stable Grik2 target:ASO duplex may indicate that an ASO is likely to be more efficacious at knocking down Grik2 mRNA expression, likely due to its increased processivity. In other words, an ASO in complex with a target sequence is more likely to disengage from less stable duplexes in order to target the same region on a different mRNA molecule, which would reflect its knockdown efficacy.

The terms “Opening Energy” and “Total Opening Energy” refer to a thermodynamic property of a nucleotide or polynucleotide (measured in kcal/mol) that corresponds to the energy required to resolve (i.e., open/render accessible) RNA secondary structure at a target location and potentially includes resolution of nearby secondary structure or involvement of distal sequences that form a secondary structure with the target sequence. In the context of the present disclosure, more negative values of the Opening Energy indicate a higher energy requirement to resolve the RNA secondary structure and reflect a reduced knockdown efficacy of a corresponding ASO sequence. This value indicates that target sequences that require less energy to unfold are more amenable to unfolding and can, therefore, be considered more accessible for ASO binding.

The terms “GC content” and “Percent (%) GC” refer to the percentage of bases in a polynucleotide (e.g., an ASO of the disclosure or a fully or a substantially complementary sequence thereof) that are either guanine (G) or cytosine (C). Unlike A-T/U bonding, which is mediated by two hydrogen bonds, G-C bonding is mediated by three hydrogen bonds. Polynucleotide duplexes with higher GC content are more stable and require more energy to resolve the duplex. This stability is not necessarily conferred by the increased number of hydrogen bonds, but rather by more stable base stacking. For a given polynucleotide, GC content may be calculated as:

$$\frac{G + C}{A + T + G + C} \times 100 \%$$

### BRIEF DESCRIPTION OF THE DRAWINGS

FIGS. 1A-1Q show the identification and assessment of glutamate ionotropic receptor kainate type subunit 2 (Grik2) mRNA antisense oligonucleotide (ASO) constructs for knocking down (KD) expression of GluK2 protein in a cell. (FIG. 1A) Predicted secondary structure of human Grik2 mRNA variant 1 (SEQ ID NO: 115) as predicted by RNAfold, centroid entropy model. (FIG. 1B) Predicted regions of binding of multiple anti-Grik2 ASOs to the Grik2 mRNA. Exemplary regions of binding include identified Loop regions (1=Loop1 (SEQ ID NO: 145); 2=Loop2 (SEQ ID NO: 146); 3=Loop3 (SEQ ID NO: 147); 4=Loop4 (SEQ ID NO: 148); 5=Loop (SEQ ID NO: 149); 6=Loop6 (SEQ ID NO: 150); 7=Unpaired 1 (SEQ ID NO: 159); 8=Unpaired 2 (SEQ ID NO: 160); 9=Loop7 (SEQ ID NO: 151); 10=Loop8 (SEQ ID NO: 152); 11=Loop9 (SEQ ID NO: 153); 12=Loop (SEQ ID NO: 154); 13=Unpaired 3 (SEQ ID NO: 161); 14=Loop11 (SEQ ID NO: 155); 15=Unpaired 4 (SEQ ID NO: 162); 16=Unpaired (SEQ ID NO: 163); 17=Loop12 (SEQ ID NO: 156); 18=Loop13 (SEQ ID NO: 157); and 19=Loop14 (SEQ ID NO: 158)) and stem-like Unpaired regions (designated by Arabic numerals 15-19 corresponding to SEQ ID NOs: 159-163, respectively). (FIG. 10) Schematic of anti-Grik2 ASO agents aligned to predicted regions of binding within the 5′ UTR (SEQ ID NO: 126; siRNA D3 (SEQ ID NO: 48), siRNA XZ (SEQ ID NO: 54), siRNA CY (SEQ ID NO: 43), siRNA D1 (SEQ ID NO: 46), siRNA GE (SEQ ID NO: 65), siRNA CX (SEQ ID NO: 42), siRNA YO (SEQ ID NO: 55), siRNA TG (SEQ ID NO: 23), siRNA D0 (SEQ ID NO: 45), siRNA YB (SEQ ID NO: 67), siRNA GF (SEQ ID NO: 64), siRNA TD (SEQ ID NO: 26), siRNA GH (SEQ ID NO: 66), siRNA TE (SEQ ID NO: 25), siRNA TJ (SEQ ID NO: 21), siRNA TF (SEQ ID NO: 24), siRNA YB/siSPOTR(SEQ ID NO: 67), siRNA ZZ/siSPOTR16 (SEQ ID NO: 100), siRNA GE/siSPOTR17 (SEQ ID NO: 65), siRNA D3/siSPOTR18 (SEQ ID NO: 48), siRNA CX/siSPOTR19 (SEQ ID NO: 42), siRNA GF/siSPOTR(SEQ ID NO: 64), siRNA GH/siSPOTR21 (SEQ ID NO: 66), siRNA TJ/siSPOTR22 (SEQ ID NO: 21), siRNA TG/siSPOTR23 (SEQ ID NO: 23), siRNA TD/siSPOTR24 (SEQ ID NO: 26), and siRNA TF/siSPOTR(SEQ ID NO: 24)) and coding sequence (CDS) of exon 1 (SEQ ID NO: 129; siRNA CK (SEQ ID NO: 29), siRNA TC (SEQ ID NO: 28), and siRNA TC/siSPOTR1 (SEQ ID NO: 28)) of the Grik2 mRNA (SEQ ID NO: 115). (FIG. 1 D) Schematic of an exemplary ASO agent (GO; SEQ ID NO: 1) aligned relative to an identified Loop1 region (SEQ ID NO: 145) within exon 2 (SEQ ID NO: 130) of the Grik2 mRNA (SEQ ID NO: 115). (FIG. 1E) Schematic of five exemplary ASO sequences (GD (SEQ ID NO: 7), MU (SEQ ID NO: 96), MT (SEQ ID NO:99), MS (SEQ ID NO: 99), and G3 (SEQ ID NO: 8)) aligned relative to identified Loop (SEQ ID NO: 149) and Loop6 (SEQ ID NO: 150) regions within exon (SEQ ID NO: 138) of the Grik2 mRNA (SEQ ID NO: 115). (FIG. 1F) Schematic showing exemplary ASO agents (MJ (SEQ ID NO: 89), TH (SEQ ID NO: 22), MI (SEQ ID NO: 90), Y9 (SEQ ID NO: 88), TK (SEQ ID NO: 74), Y8 (SEQ ID NO: 87), TI (SEQ ID NO: 76), CU (SEQ ID NO: 39), and Y7 (SEQ ID NO: 62)) aligned relative to exon 11 (SEQ ID NO: 139) of the Grik2 mRNA (SEQ ID NO: 115). (FIG. 1G) Percent reporter knockdown of Grik2 mRNA by various candidate ASO agents in a dual-luciferase reporter assay (see also Table 2). (FIG. 1H) Non-specific firefly luciferase (ffluc) reduction for various candidate anti-Grik2 ASO agents in a dual-luciferase reporter assay. (FIG. 11) Scatter plot showing percent Grik2 mRNA knockdown as a function of residual ffluc expression from an empty control vector (relationship targeted and non-specific ffluc knockdown). (FIG. 1J) Bar plot of the mean (with SEM) value of Target Opening Energy in kcal/mol for all 19 bp siRNAs tested in a luciferase reporter assay. Data bars (middle and right bars) were separated by siRNAs that knocked down reporter-induced Gluk2 expression by greater than 66%, or siRNAs that knocked down expression by less than 66%. An enriched cluster of siRNAs having a Target Opening Energy of less than kcal/mol correlated with greater than 66% Gluk2 knockdown. (FIG. 1K) Bar plot of the mean (with SEM) value of Target Opening Energy in kcal/mol for all 21 bp guides against the percent knockdown of their 19 bp equivalents as tested in a luciferase reporter assay. Data bars (middle and right bars) were separated by equivalent siRNAs that knocked down reporter-induced Gluk2 expression by greater than 66%, or equivalent siRNAs that knocked down expression by less than 66%. An enriched cluster of siRNAs having a Target Opening Energy of less than 9.5 kcal/mol correlated with greater than 66% Gluk2 knockdown. (FIG. 1L) Bar plot of the mean (with SEM) of energy of duplex formation in kcal/mol of all 19 bp siRNAs tested in a luciferase reporter assay. Data bars represent from left to right: all 19 bp siRNAs tested, siRNAs that knocked down reporter expression by >66%, or siRNAs that knocked down reporter expression by <66%. (FIG. 1M) Bar plot of the mean (with SEM) value of Energy of Duplex Formation in kcal/mol for all 21 bp guides against the percent knockdown of their 19 bp equivalents as tested in a luciferase reporter assay. Data bars (middle and right bars) were separated by equivalent siRNAs that knocked down reporter-induced Gluk2 expression by greater than 66%, or equivalent siRNAs that knocked down expression by less than 66%. (FIG. 1N) Bar plot of the mean (with SEM) of Total Energy of Binding in kcal/mol for all 19 bp siRNAs tested in a luciferase reporter assay. Data bars represent from left to right: all 19 bp siRNAs tested, siRNAs that knocked down reporter expression by >66%, or siRNAs that knocked down reporter expression by <66%. (FIG. 1O) Bar plot of the mean (with SEM) value of Total Energy of Binding in kcal/mol for all 21 bp guides against the percent knockdown of their 19 bp equivalents as tested in a luciferase reporter assay. Data bars (middle, right) were separated by equivalent siRNAs that knocked down reporter-induced Gluk2 expression by greater than 66%, or equivalent siRNAs that knocked down expression by less than 66%. (FIG. 1 P) Bar plot of the mean (with SEM) of percent of bases identified as G or C (GC content) in each of the 19 bp siRNAs tested in a luciferase reporter assay. Data bars represent from left to right: all 19 bp siRNAs tested, siRNAs that knocked down reporter expression by >66%, or siRNAs that knocked down reporter expression by <66%. (FIG. 1Q) Bar plot of the mean (with SEM) value of GC content for all 21 bp guides against the percent knockdown of their 19 bp equivalents as tested in a luciferase reporter assay. Data bars (middle, right) were separated by equivalent siRNAs that knocked down reporter-induced Gluk2 expression by greater than 66%, or equivalent siRNAs that knocked down expression by less than 66%.

FIGS. 2A-2J show the validation of GluK2 knockdown by viral vector-mediated Grik2 mRNA silencing. (FIGS. 2A-2D) Exemplary vectors utilized in the experiments described in FIGS. 2A-2J. (FIG. 2A) Exemplary lentiviral plasmid map for a lentiviral vector (CM845) encoding a control scramble sequence (SEQ ID NO: 771) under control of an hSyn promoter (SEQ ID NO: 682). (FIG. 2B) Exemplary lentiviral plasmid map for a lentiviral vector (CM946) encoding a Grik2 antisense sequence (G9; SEQ ID NO: 68) as an shRNA under control of a U6 promoter (SEQ ID NO: 772). (FIG. 2C) Exemplary lentiviral plasmid map for a lentiviral vector (CM962) encoding a Grik2 antisense sequence (G9; SEQ ID NO: 68) as a miRNA under control of an hSyn promoter (SEQ ID NO: 683). (FIG. 2D) Exemplary plasmid map for an AAV vector encoding GFP under control of an hSyn promoter (pAAV-hSyn-EGFP; SEQ ID NO: 682). (FIG. 2E) Brightfield (left panel) and fluorescence (right panel) imaging of cultured rat hippocampal neurons infected with a lentiviral (LV)-human synapsin promoter (hSyn (SEQ ID NO: 682))-green fluorescent protein (GFP) plasmid construct. GFP immunofluorescence was observed in >80% of cultured neurons. (FIG. 2F) Western blot of GluK2 protein and actin obtained from rat hippocampal neurons following lentivirally-mediated knockdown of Grik2 mRNA with either a short-hairpin RNA (LV-U6 (SEQ ID NO: 772)-G9 (shRNA) or a microRNA (LV-hSyn (SEQ ID NO: 683)-G9 (miRNA) construct encoding a Grik2 antisense sequence (G9; SEQ ID NO: 68) or a control sequence (SEQ ID NO: 771; under control of an hSyn promoter (SEQ ID NO: 682)). (FIG. 2G) Schematic of an AAV expression cassette used for AAV-mediated viral transduction in cells. (FIG. 2H) Bar graph representing relative levels of GluK2 protein versus actin normalized to the value in control conditions for hippocampal neurons infected with the lentiviral or AAV9 vectors encoding an anti-Grik2 ASO sequence (G9; SEQ ID NO: 68) or a scrambled control sequence (LV: SEQ ID NO: 771; AAV: GC—SEQ ID NO: 101). (FIG. 2I) Plot showing relative levels of GluK2 protein, as assayed by Western blot, in murine primary cortical neurons treated with different virally-encoded anti-Grik2 ASO sequences (G9 (SEQ ID NO: 68); GI (SEQ ID NO: 77); XY (SEQ ID NO: 83); Y9 (SEQ ID NO: 88); GG (SEQ ID NO: 91)) and control sequence (GC; SEQ ID NO: 101). (FIG. 2J) shows a bar plot of fold change in Grik2 mRNA expression measured days following lipid-based transfection of induced pluripotent stem cell (iPSC)-derived glutamatergic neurons (GlutaNeurons) cultured at a cell density of 17,500 cells/well (17.5k c/w) with plasmid vectors encoding one of five Grik2 mRNA antisense oligonucleotides (G9 (SEQ ID NO: 68), GI (SEQ ID NO: 77), Y9 (SEQ ID NO: 88), XY (SEQ ID NO: 83), or MU (SEQ ID NO: 96)) or a scrambled control sequence (GC; SEQ ID NO: 101) under regulatory control of an hSyn promoter (SEQ ID NO: 683), as measured by RT-qPCR.

FIGS. 3A-3D show the effects of virally-encoded anti-Grik2 ASO agents on hippocampal epileptiform activity in a murine in vitro model. (FIG. 3A) Fluorescence images showing organotypic hippocampal brain slices infected with an AAV9-hSyn (SEQ ID NO: 682)-GFP-scramble construct containing a scrambled sequence (SEQ ID NO: 101). The slice was immuno-stained with a Prospero Homeobox Protein 1 (Prox1) antibody (Millipore) to label dentate gyrus (DG) cells of the hippocampus. (FIG. 3B) Exemplary extracellular voltage trace of an ED recorded from a murine organotypic hippocampal slice. (FIG. 3C) Bar graph representing the frequency of EDs in murine hippocampal slices infected with lentiviral or AAV9 vectors encoding an anti-Grik2 ASO sequence as an miRNA construct (G9; SEQ ID NO: 68; ***, p<0.001; **, p<0.01) or a scramble control sequence (AAV-GC (SEQ ID NO: 101); LV-scramble (SEQ ID NO: 771)) under control of an hSyn promoter (LV and AAV9-GC: SEQ ID NO: 682; AAV9-hSyn-G9: SEQ ID NO: 683), or an AAV9-GFP control vector. (FIG. 3D) Bar graph representing the frequency of EDs in murine organotypic hippocampal slices treated with the indicated AAV9-encoded anti-Grik2 ASO agents (AAV9-hSyn (SEQ ID NO: 683)-G9 (SEQ ID NO: 68)−p =0.0004; AAV9-hSyn (SEQ ID NO: 683)-XY (SEQ ID NO: 91)−p=0.0008; AAV9-hSyn (SEQ ID NO: 683)-GI (SEQ ID NO: 85)−p=0.0478); AAV9-hSyn (SEQ ID NO: 683)-Y9 (SEQ ID NO: 96); and AAV9-hSyn (SEQ ID NO: 683)-GG (SEQ ID NO: 91) or control scramble sequence and a GFP tag (AAV9-hSyn (SEQ ID NO: 682)-GFP-GC (SEQ ID NO: 101)).

FIGS. 4A-4F show the efficacy of Grik2-targeting ASO agents in an in vivo murine model of temporal lobe epilepsy (TLE). (FIG. 4A) Schematic for the experimental design of a Novel Object Recognition (NOR) task. (FIG. 4B) Bar graph showing the discrimination index (DI) of mice subjected to a NOR task, as measured 7 days prior to injection or days after injection with either a virally-encoded scrambled sequence (GC; SEQ ID NO: 101; AAV9-hSyn (SEQ ID NO: 682)-GFP-GC) or an anti-Grik2 sequence (G9; SEQ ID NO: 68; AAV9-hSyn (SEQ ID NO: 683)-G9). (FIG. 4C) Bar graph showing the total distance traveled (cm) by mice in a NOR task, as measured 7 days prior to injection or days after injection with either a virally-encoded scrambled sequence (GC; SEQ ID NO: 101) or an anti-Grik2 sequence (G9; SEQ ID NO: 68). (FIG. 4D) Exemplary voltage trace of an electrographic seizure induced in a pilocarpine model of TLE, as recorded from a mouse following treatment with pilocarpine. (FIG. 4E) Bar graph showing the cumulative seizure duration (minutes) across days in mice treated with a virally-encoded scrambled control sequence (GC; SEQ ID NO: 101; n=3) or a virally-encoded anti-Grik2 ASO agent (G9; SEQ ID NO: 68; n=4). (FIG. 4F) Bar graph showing the cumulative number of seizures across days in mice treated with a virally-encoded scrambled control sequence (GC; SEQ ID NO: 101; n=5) or a virally-encoded anti-Grik2 ASO agent (G9; SEQ ID NO: 68; n=6).

FIG. 5 is a bar graph showing knockdown efficacy of various Grik2 mRNA-targeting microRNA constructs encoded in an AAV9 vector. The AAV9 vector incorporates one of microRNA scaffolds containing a 5′ flanking region, microRNA loop sequence, and 3′ flanking region from an endogenous microRNA, including A-miR-(51), E-miR-(S2), E-miR-155 (S3), E-miR-218 (S4), and E-miR-124 (S5). Antisense sequences tested were G9 (SEQ ID NO: 68), GI (SEQ ID NO: 77), MW (SEQ ID NO: 80), GU (SEQ ID NO: 96), TO (SEQ ID NO: 14), TK (SEQ ID NO: 74), TH (SEQ ID NO: 22), CQ (SEQ ID NO: 35), XU (SEQ ID NO: 51), XY (SEQ ID NO: 83), Y9 (SEQ ID NO: 88), YA (SEQ ID NO: 63), GG (SEQ ID NO: 91), G8 (SEQ ID NO: 92), ME (SEQ ID NO: 69), and MD (SEQ ID NO: 70). Knockdown efficacy is represented as Grik2 mRNA median fold change relative to “only lipid” control. This larger panel of miRNA-expressing plasmids, under the control of the hSyn promoter (SEQ ID NO: 790), was transfected into induced pluripotent stem cell (iPSC)-derived glutamatergic neurons (GlutaNeurons) and evaluated for their ability to reduce Grik2 mRNA levels by RT-qPCR. When compared to non-transfected cells (dashed line) and using a median absolute deviation (MAD)=2 to identify functional constructs (dotted line), the majority of constructs were determined to be functional (i.e., they exhibit knockdown Grik2 mRNA below MAD). Of all of the tested constructs, GI (SEQ ID NO: 77)-S2 (SEQ ID NO: 798), MW (SEQ ID NO: 80)-S4 (SEQ ID NO: 799), MW-S(SEQ ID NO: 800), and G9 (SEQ ID NO: 68)-S(SEQ ID NO: 801) were found to knockdown Grik2 mRNA to the highest degree (i.e., 20% or greater knockdown).

FIGS. 6A-6G show schematic diagrams of synthetic AAV9-miRNA construct configurations containing antisense guide sequences incorporated into an A-miR-(S1) scaffold containing a 5′ flanking region, microRNA loop sequence, and 3′ flanking region from an endogenous miRNA. Construct 1 (FIG. 6A) is a single-miRNA, single promoter construct (SEQ ID NO: 775) containing from 5′ to 3′: a 5′ ITR sequence (SEQ ID NO: 746), hSyn promoter sequence (SEQ ID NO: 790), miR-30 5′ flanking sequence (SEQ ID NO: 752), a passenger strand sequence substantially complementary to the anti-Grik2 sequence of G9 (SEQ ID NO: 68), a miR-loop sequence (SEQ ID NO: 758), guide sequence of G9 (SEQ ID NO: 68), miR-30 3′ flanking sequence (SEQ ID NO: 753), a rabbit beta-globin (RBG) polyA signal (SEQ ID NO: 792), and a 3′ ITR sequence (SEQ ID NO: 789). Construct 2 (FIG. 6B) is a single miRNA, dual promoter construct (SEQ ID NO: 777) containing, from 5′ to 3′: a 5′ ITR sequence (SEQ ID NO: 746), C1ql2 promoter sequence (SEQ ID NO: 791), hSyn promoter sequence (SEQ ID NO: 790), miR-30 5′ flanking sequence (SEQ ID NO: 752), a passenger strand sequence substantially complementary to the anti-Grik2 sequence of G9 (SEQ ID NO: 68), a miR-loop sequence (SEQ ID NO: 785), guide sequence of G9 (SEQ ID NO: 68), miR-30 3′ flanking sequence (SEQ ID NO: 753), RBG polyA signal (SEQ ID NO: 792), and a 3′ ITR sequence (SEQ ID NO: 748). Construct 3 (FIG. 6C) is a self-complementary, dual-miRNA (two copies of G9, SEQ ID NO: 68), single promoter construct (SEQ ID NO: 779) containing a wild-type AAV (wt)ITR at the 5′ end adjacent (i.e., 5′) to a hSyn promoter (SEQ ID NO: 790) and a mutant ITR (mITR) downstream of a polyA sequence. Construct 4 (FIG. 6D; SEQ ID NO: 781) is similar to Construct 3, except that the hSyn promoter (SEQ ID NO: 790) is adjacent to the mITR and the polyA sequence is adjacent to the wtITR. Construct (SEQ ID NO: 783) and Construct 6 (SEQ ID NO: 784) are similar to Construct 1, except that the pre-miR stem-loop structure (5′ flank, stem-loop, and 3′ flank) is concatemerized three times, such that the construct contains three copies of the same miRNA sequence (e.g., G9, SEQ ID NO: 68; Construct 5) or three copies of a different miRNA sequence (FIG. 6E; Construct 6; G9, GI (SEQ ID NO: 77), MU (SEQ ID NO: 96)). Construct 7 (FIG. 6F; SEQ ID NO: 804) is a single-miRNA, single promoter construct containing from 5′ to 3′, a 5′ ITR sequence (SEQ ID NO: 746), hSyn promoter sequence (SEQ ID NO: 790), miR-30 5′ flanking sequence (SEQ ID NO: 752), a passenger strand sequence substantially complementary to the anti-Grik2 sequence of G9 (SEQ ID NO: 68), a miR-loop sequence (SEQ ID NO: 758), guide sequence of G9 (SEQ ID NO: 68), miR-30 3′ flanking sequence (SEQ ID NO: 753), RBG polyA signal (SEQ ID NO: 792), a non-coding stuffer sequence, and a 3′ ITR sequence (SEQ ID NO: 789). Construct 8 (FIG. 6G; SEQ ID NO: 810) is a single-miRNA, single promoter construct containing from 5′ to 3′, a 5′ ITR sequence (SEQ ID NO: 746), hSyn promoter sequence (SEQ ID NO: 790), E-miR-124-3 5′ flanking sequence (SEQ ID NO: 768), a sense passenger strand sequence that is complementary to the antisense sequence of G9 (SEQ ID NO: 68), E-miR-124-3 loop sequence (SEQ ID NO: 770), antisense guide sequence of G9, E-miR-124-3 3′ flanking sequence (SEQ ID NO: 769), RBG polyA signal (SEQ ID NO: 792), a non-coding stuffer sequence, and a 3′ ITR sequence (SEQ ID NO: 789).

FIG. 7 is a photograph showing alkaline agarose gel electrophoresis analysis of single- and dual-miRNA expression constructs (Constructs 1-6) described in FIGS. 6A-6E. The genome content of a vector produced from a plasmid encoding a single promoter and a single miRNA cassette (expected length: 1.5 kb) was found to be comprised of a mixture of singly (1.5 kb), doubly (3.0 kb), and triply (4.5 kb) packaged genomes. Lane numbers correspond to the following vector constructs: 1=Construct 1 (SEQ ID NO: 775); 2=Construct 2 (SEQ ID NO: 777); 3=Construct 3 (SEQ ID NO: 779); 4=Construct 4 (SEQ ID NO: 781); 5=Construct (SEQ ID NO: 783); 6=Construct 6 (SEQ ID NO: 784).

FIGS. 8A-8G show schematic diagrams of AAV9 dual-miRNA expression constructs having dual promoters suitable for use with AAV vectors. FIG. 8A shows a dual-miRNA dual promoter vector (DMTPV1) expression construct (SEQ ID NO: 785) containing, from 5′ to 3′, a 5′ ITR sequence (SEQ ID NO: 746), hSyn promoter (SEQ ID NO: 790), E-miR-124-3 5′ flanking sequence (SEQ ID NO: 768), a sense passenger (“P”) strand sequence that is complementary to the antisense sequence of G9 (SEQ ID NO: 68), E-miR-124-3 loop sequence (SEQ ID NO: 770), antisense guide (“G”) sequence of G9, E-miR-124-3 3′ flanking sequence (SEQ ID NO: 769), BGH polyA sequence (SEQ ID NO: 793), CaMKII promoter sequence (SEQ ID NO: 802), E-miR-30 5′ flanking sequence (SEQ ID NO: 759), a sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), RBG polyA sequence (SEQ ID NO: 792), and a 3′ ITR sequence (SEQ ID NO: 748). FIG. 8B shows a dual siRNA expression construct (DMTPV2, SEQ ID NO: 786) containing, from 5′ to 3′, a 5′ ITR sequence (SEQ ID NO: 746), hSyn promoter (SEQ ID NO: 790), E-miR-124-3 5′ flanking sequence (SEQ ID NO: 768), a sense passenger strand sequence that is complementary to the antisense sequence of G9 (SEQ ID NO: 68), E-miR-124-3 loop sequence (SEQ ID NO: 770), antisense guide sequence of G9, E-miR-124-3 3′ flanking sequence (SEQ ID NO: 769), BGH polyA sequence (SEQ ID NO: 793), CaMKII promoter sequence (SEQ ID NO: 802), E-miR-218 5′ flanking sequence (SEQ ID NO: 765), a sense passenger strand sequence that is complementary to MW (SEQ ID NO: 80), E-miR-218 loop sequence (SEQ ID NO: 767), antisense guide sequence of MW (SEQ ID NO: 80), E-miR-218 3′ flanking sequence (SEQ ID NO: 766), RBG polyA sequence (SEQ ID NO: 792), and 3′ ITR sequence (SEQ ID NO: 748). FIG. 8C shows a dual siRNA expression construct (DMTPV3, SEQ ID NO: 787) containing, from 5′ to 3′, a 5′ ITR sequence (SEQ ID NO: 746), hSyn promoter (SEQ ID NO: 790), E-miR-30 5′ flanking sequence (SEQ ID NO: 759), a sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), BGH polyA sequence (SEQ ID NO: 793), CaMKII promoter sequence (SEQ ID NO: 802), E-miR-124-3 5′ flanking sequence (SEQ ID NO: 768), a sense passenger strand sequence that is complementary to the antisense sequence of G9 (SEQ ID NO: 68), E-miR-124-3 loop sequence (SEQ ID NO: 770), antisense guide sequence of G9, E-miR-124-3 3′ flanking sequence (SEQ ID NO: 769), RBG polyA sequence (SEQ ID NO: 792), and 3′ ITR sequence (SEQ ID NO: 748). FIG. 8D shows a dual siRNA expression construct (DMTPV4, SEQ ID NO: 788) containing, from 5′ to 3′, a 5′ ITR sequence (SEQ ID NO: 746), hSyn promoter (SEQ ID NO: 790), E-miR-30 5′ flanking sequence (SEQ ID NO: 759), a sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), BGH polyA sequence (SEQ ID NO: 793), CaMKII promoter sequence (SEQ ID NO: 802), E-miR-124-3 5′ flanking sequence (SEQ ID NO: 768), a sense passenger strand sequence that is complementary to the antisense sequence of MW (SEQ ID NO: 30 80), E-miR-124-3 loop sequence (SEQ ID NO: 770), antisense guide sequence of MW (SEQ ID NO: 80), E-miR-124-3 3′ flanking sequence (SEQ ID NO: 769), RBG polyA sequence (SEQ ID NO: 792), and 3′ ITR sequence (SEQ ID NO: 748). FIG. 8E shows a dual siRNA expression construct (DMTPVS) containing, from 5′ to 3′, a 5′ ITR sequence (SEQ ID NO: 746), hSyn promoter (SEQ ID NO: 790), E-miR-30 5′ flanking sequence (SEQ ID NO: 759), a sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), BGH polyA sequence (SEQ ID NO: 793), CaMKII promoter sequence (SEQ ID NO: 802), E-miR-218 5′ flanking sequence (SEQ ID NO: 765), a sense passenger strand sequence that is complementary to the antisense sequence of MW (SEQ ID NO: 80), E-miR-218 loop sequence (SEQ ID NO: 767), antisense guide sequence of MW (SEQ ID NO: 80), E-miR-218 3′ flanking sequence (SEQ ID NO: 766), RBG polyA sequence (SEQ ID NO: 792), and 3′ ITR sequence (SEQ ID NO: 748). FIG. 8F shows a dual siRNA expression construct (DMTPV6) containing, from 5′ to 3′, a 5′ ITR sequence (SEQ ID NO: 746), hSyn promoter (SEQ ID NO: 790), E-miR-30 5′ flanking sequence (SEQ ID NO: 759), sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), E-miR-218 5′ flanking sequence (SEQ ID NO: 765), sense passenger strand sequence that is complementary to the antisense sequence of MW (SEQ ID NO: 80), E-miR-218 loop sequence (SEQ ID NO: 767), antisense guide sequence of MW (SEQ ID NO: 80), E-miR-218 3′ flanking sequence (SEQ ID NO: 766), RBG polyA sequence (SEQ ID NO: 792), non-coding stuffer sequence, and 3′ ITR sequence (SEQ ID NO: 748). FIG. 8G shows a dual siRNA expression construct (DMTPV7) containing, from 5′ to 3′, a 5′ ITR sequence (SEQ ID NO: 746), hSyn promoter (SEQ ID NO: 790), E-miR-30 5′ flanking sequence (SEQ ID NO: 759), sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), BGH polyA sequence (SEQ ID NO: 793), CaMKII promoter sequence (SEQ ID NO: 802), E-miR-124-3 5′ flanking sequence (SEQ ID NO: 768), antisense guide sequence of G9, E-miR-124-3 loop sequence (SEQ ID NO: 770), sense passenger strand sequence that is complementary to the antisense sequence of G9 (SEQ ID NO: 68), E-miR-124-3 3′ flanking sequence (SEQ ID NO: 769), RBG polyA sequence (SEQ ID NO: 792), and 3′ ITR sequence (SEQ ID NO: 748).

FIGS. 9A and 9B are photographs showing alkaline agarose gel analysis of cDNA of vectors produced from single-siRNA vector constructs (FIG. 9A; G9, SEQ ID NO: 68 - Construct 1 (SEQ ID NO: 775); GC, SEQ ID NO: 101) and dual-miRNA vector constructs (FIG. 9B; DMTPV1-4; SEQ ID NOs: 785-788, respectively). Single bands across all four dual-miRNA vector constructs indicates that vectors of dual expression constructs are singly-packaged in AAV9 vectors.

FIG. 10 shows a bar graph demonstrating the in vitro efficacy of GluK2 protein knockdown using single-miRNA AAV9 constructs delivered singly or in combination with another single-miRNA AAV9 construct containing a different miRNA sequence (G9-S1 (SEQ ID NO: 775), GI-S1 (SEQ ID NO: 796), GI-S2 (SEQ ID NO: 798), MW-S4 (SEQ ID NO: 799), G9-S(SEQ ID NO: 800) or combinations thereof), as measured by qPCR. GlutaNeurons transfected with combinations of two different anti-Grik2 miRNA sequences (both under control of the hSyn promoter (SEQ ID NO: 790)) showed similar knockdown of GluK2 protein as GlutaNeurons transfected with a single type of anti-Grik2 miRNA sequence, supporting the use of vectors encoding more than one unique antisense guide sequence against Grik2 to knockdown GluK2 expression. Knockdown efficacy was measured as a fold-change in median Grik2 mRNA level fold-change relative to “Lipid only” control group.

FIG. 11 shows a bar graph of the frequency of epileptiform activity in disinhibited murine organotypic hippocampal slices transfected with combinations of different single-miRNA AAV9 expression vectors (GC (SEQ ID NO: 101); G9-S1 (SEQ ID NO: 775), GI-S1 (SEQ ID NO: 796), or G9-S1 +GI-S1) under control of a hSyn promoter (SEQ ID NO: 790). Combinations of miRNA constructs, G9-S1 and GI-S1, showed an equivalent degree of suppression of epileptiform activity as each vector individually, supporting the use of more than one unique antisense guide sequence against Grik2 to suppress epileptiform activity in hippocampal circuits.

FIG. 12 shows a bar graph representing levels of Grik2 mRNA, as measured by qPCR, following AAV9 vector-mediated knockout of Grik2 in GlutaNeurons using one of several antisense constructs, including hSyn.Gl (SEQ ID NO: 77). S2 (SEQ ID NO: 798), hSyn.MW (SEQ ID NO: 80).S4 (SEQ ID NO: 799), hSyn.MW.S(SEQ ID NO: 800), hSyn.G9 (SEQ ID NO: 68).55 (SEQ ID NO: 801), CaMKII.GI.S4, CaMKII.MW.S5, CaMKII.G9.S5, DMTPV1 (SEQ ID NO: 785), DMTPV2 (SEQ ID NO: 786), DMTPV3 (SEQ ID NO: 787), and DMTPV4 (SEQ ID NO: 788). mRNA levels were compared to GlutaNeurons transduced with an AAV.null vector containing a full-length genome that does not produce RNA.

FIGS. 13A and 13B show the results of an open field test conducted with mice treated with pilocarpine and one of several single-miRNA vector constructs (GC (SEQ ID NO: 101), G9-S1 (SEQ ID NO: 775), or GI-S1 (SEQ ID NO: 796)). FIG. 13A shows an exemplary trace of tracked locomotion for a single mouse in an open field. FIG. 13B shows a bar graph demonstrating the total distance traveled in an open field test by mice treated with AAV9 vectors encoding an anti-Grik2 miRNA sequence. Non-epileptic mice (i.e., mice not treated with pilocarpine; n=20), and chronic epileptic mice treated with GC, G9-S1 or GI-S1(n=9, n=8, n=9, respectively). Pre- and post-injection data were compared using a Mann-Whitney test, *p<0.05 and p<0.01. Note the significant reduction of hyperlocomotion with G9 and GI.

FIG. 14 is a bar graph showing the total number of epileptic seizures per day in pilocarpine-treated mice treated with an AAV9 vector encoding the anti-Grik2 construct G9-S1 (SEQ ID NO: 775), GI-15 51 (SEQ ID NO: 796), or scrambled control construct GC (SEQ ID NO: 101)(n=5, n=5, n=5, respectively). G9-S1 and GI-S1 raw data were compared with GC using a one-way ANOVA test, p<0.01. Note the suppression of seizures with G9-S1 and GI-S1.

FIG. 15 is a bar graph showing the total distance traveled in an open field test by mice treated with an AAV9 vector encoding the anti-Grik2 construct G9 (SEQ ID NO: 68) at different doses. Chronic epileptic mice treated with different doses of G9: G9/1, G9/10, G9/100 and G9/1000 (n=8, n=5, n=5, n=5, respectively). GC is the control construct (n=9). Pre- and post-injection data were compared using a Mann-Whitney test, *p<0.05 and p<0.01. Note the similar effect of G9 and G9/10.

FIG. 16 is a bar graph showing the total number of epileptic seizures per day in pilocarpine-treated mice treated with an AAV9 vector encoding the anti-Grik2 construct G9-S1/G9 (SEQ ID NO: 775) at one of several doses: G9/1, G9/10, G9/100 and G9/1000 (n=6, n=4, n=2, n=2, respectively). Note the similar effect with G9 and G9/10, but not with G9/1000.

FIG. 17 is a bar graph showing the total distance traveled in an open field test by pilocarpine-treated mice treated with an AAV9 vector encoding one of several dual-miRNA, dual promoter constructs (DMTPV1-4). Non-epileptic mice (n=20) and chronic epileptic mice treated with DMTPV1 (SEQ ID NO: 785), DMTPV2 (SEQ ID NO: 786), DMTPV3 (SEQ ID NO: 787), or DMTPV4 (SEQ ID NO: 788)(n=6, n=5, n=6 and n=6, respectively). Pre- and post-injection data were compared using a Mann-Whitney test, *p<0.05 and p<0.01. Note the significant reduction of hyperlocomotion with DMTPV3 and DMTPV4.

FIG. 18 is a scatter plot showing the total distance traveled (cm) in an open field test versus the number of spontaneous epileptic seizures per day in pilocarpine-treated mice. Regression analysis demonstrates a significant correlation between hyperlocomotion and seizure susceptibility (R²=0.7388, p<0.0001).

FIG. 19 is a bar graph showing Grik2 mRNA expression following transduction of GlutaNeurons with one of several anti-Grik2 miRNA sequences, including G9 (SEQ ID NO: 68), GI (SEQ ID NO: 77), DMTPV1 (SEQ ID NO: 785), DMTPV2 (SEQ ID NO: 786), DMTPV3 (SEQ ID NO: 787), and DMTPV4 (SEQ ID NO: 788), and a control AAV9.hSyn.GFP vector. All tested dual-miRNA constructs reduced Grik2 mRNA levels in GlutaNeurons, as measured using RNA sequencing. Fold change is relative to control.

FIG. 20 is a bar graph showing locomotion in an open field test of mice treated with a vector encoding the single anti-Grik2 miRNA sequence G9 (SEQ ID NO: 68), dual-miRNA vector DMTPV3 (SEQ ID NO: 787), or mice treated with a control AAV9.hSyn.GFP vector. WT mice were used as a separate control. Pre=pre-treatment; Post=Post-treatment. Both G9- and DMTPV3-encoding vectors significantly suppressed hyperlocomotor activity in mice (p<0.01; Mann-Whitney test), suggesting a robust effect of these vectors on suppression of hyperlocomotion.

FIG. 21 is a bar graph showing a dose-dependent reduction in hyperlocomotor activity in an open field test in mice treated with varying doses of DMTPV3 (SEQ ID NO: 787), including DMTPV3 (3.6×10¹⁰ GC/brain), DMTPV3/10 (3.6×10⁹ GC/brain), DMTPV3/100 (3.6×10⁸ GC/brain), and DMTPV3/1000 (3.6×10⁷ GC/brain). Control mice were treated with an AAV9.hSyn.GFP vector (3.6×10¹⁰ GC/brain). Pre=pre-treatment; Post=Post-treatment. Mice treated with DMTPV3 and DMTPV3/10 doses showed a significant reduction in hyperlocomotor activity relative to control mice (p<0.01; Mann-Whitney test).

FIG. 22 is a bar graph showing number of seizures per day in pilocarpine-treated mice further treated with vectors encoding anti-Grik2 single-miRNA constructs G9 (SEQ ID NO: 68) or GI (SEQ ID NO: 77), or a dual-miRNA construct DMTPV3 (SEQ ID NO: 787). Mice were also treated with control vectors encoding a scrambled RNA sequence GC (SEQ ID NO: 101) or AAV9.hSyn.GFP. Mice treated with G9, GI, and DMTPV3 showed a significant reduction in the number of seizures per day, with DMTPV showing greater reduction as compared to G9 and GI (* p<0.05; **p<0.01; Mann-Whitney test).

FIG. 23 shows fluorescence images of organotypic hippocampal brain slices resected from a human patient with TLE that were infected with AAV9.GC(SEQ ID NO: 101).GFP following DIV 1 and stained for markers of dentate granule cells (PROX1). PROX1 labeling was observed in dentate granule cells of the dentate gyrus. GFP labeling was also observed in dentate granule cells. Many cells showed co-labeling of PROX1 and GFP, indicating that AAV9.GC.GFP was able to robustly transduce dentate granule cells.

FIG. 24 is a scatter plot showing the efficacy of GluK2 protein knockdown using AAV9 expression vectors encoding an anti-Grik2 miRNA sequence (G9; SEQ ID NO: 68; n=17 slices from six subjects) or GI (SEQ ID NO: 77; two slices from two subjects) in resected hippocampal tissue from human TLE patients. Knockdown of GluK2 protein expression was observed in five out of five sets of human hippocampal tissue treated with a G9-encoding vector.

FIG. 25 shows an image of a western blot gel showing GluK2 protein expression from organotypic hippocampal slices resected from human patients with TLE and treated with a vector encoding G9 (SEQ ID NO: 68). G9 was able to reduce GluK2 protein expression by 40% relative to untreated slices. GluK2 expression was normalized to control.

FIGS. 26A-26C show illustrative local field potential recordings from organotypic hippocampal slices from a human patient with TLE under physiological conditions (ACSF) and quantification of recorded epileptiform discharges. Slices were treated with a vector encoding anti-Grik2 sequence G9-S1 (SEQ ID NO: 775; 7 slices; FIG. 26A) or a vector encoding a scrambled sequence GC and a GFP reporter (SEQ ID NO: 101; 6 slices; FIG. 26B). Insets show voltage traces of individual epileptiform discharges with higher temporal resolution. Across slices obtained from four human TLE patients, G9-S1 was able to effectively reduce or completely eliminate occurrence of epileptiform discharges (FIG. 26C; **p<0.01).

FIG. 27A-27D show suppression of Grik2 expression in organotypic hippocampal slices resected from a human TLE patient. Scatter plot of Grik2 gene expression in human organotypic hippocampal slices treated with DMTPV3 (SEQ ID NO: 787) or AAV.hSyn.GFP control vector. DMTPV3 showed a substantial reduction in Grik2 levels, as measured by qRT-PCR (FIG. 27A). Exemplary voltage trace recorded from a resected organotypic hippocampal brain slice obtained from a human TLE patient treated with a vector encoding DMTPV3 or a scrambled control sequence GC (SEQ ID NO: 101; FIG. 27B). Each asterisk represents an epileptiform discharge. Inset shows a zoomed in trace of a single epileptiform discharge. Slices treated with DMTPV3 showed a complete elimination of epileptiform discharges (FIG. 27C). Bar graph showing quantification of the frequency of epileptiform discharges recorded in slices treated with GC, G9, AAV9.hSyn.GFP, or DMTPV3 (FIG. 27D). Note that the first two bars (corresponding to GC- and G9-treated groups) are the same as those shown in FIG. 26C and are included for comparison to the DMTPV3-treated group.

FIG. 28 is a bar graph depicting the percentage of GluK2 expression in mouse cortical neurons treated with expression vectors DMSPV1 (SEQ ID NO: 811) and DMTPV8 (SEQ ID NO: 813) relative to a control AAV9.hSyn.GFP vector and a hSyn.G9-A-miR-benchmark vector (SEQ ID NO: 775). Data are presented as mean±S.E.M. Expression constructs DMSPV1 and DMPTV8 led to knockdown of GluK2 in mouse cortical neurons comparable to the benchmark hSyn.G9-A-miR-vector.

FIG. 29 is a bar graph depicting the total distance traveled by chronic epileptic mice during minutes of spontaneous exploration in an open field box before (unfilled bars) and after (filled bars) treatment with a control vector AAV9.hsyn.GFP (3.6E+9 MOI; n=2), DMSPV1 (3.6E+9 or 3.6E+8 MOI; n=3 for each MOI), DMTPV8 (3.6E+9 or 3.6E+8 MOI; n=3 for each MOI). DMSPV1 and DMTPV8 produced a dose-dependent reduction in hyperlocomotor activity in mice, which is a behavioral proxy for epileptogenesis.

FIG. 30 is a schematic diagram of an AAV vector of the disclosure containing, from 5′ to 3′:
(a) an AAV 5′ ITR sequence (e.g., any one of SEQ ID NOs: 746 and 747);
(b) a promoter sequence, such as, for example, any one of:
   (i) hSyn promoter (e.g., any one of SEQ ID NOs: 682, 683, 684, and 685);
   (ii) NeuN promoter (e.g., SEQ ID NO: 686);
   (iii) CaMKII promoter (e.g., any one of SEQ ID NOs: 687-691 and 802);
   (iv) NSE promoter (e.g., SEQ ID NO: 692 or 393);
   (v) PDGF-beta promoter (e.g., any one of SEQ ID NOs: 694-696);
   (vi) VGIuT promoter (e.g., any one of SEQ ID NOs: 697-701);
   (vii) SST promoter (e.g., SEQ ID NO: 702 or 703);
   (viii) NPY promoter (e.g., SEQ ID NO: 704);
   (ix) VIP promoter (e.g., SEQ ID NO: 705 or 706);
   (x) PV promoter (e.g., any one of SEQ ID NO: 707-709);
   (xi) GAD65 promoter (e.g., any one of SEQ ID NOs: 710-713);
   (xii) GAD67 promoter (e.g., SEQ ID NO: 714 or 715);
   (xiii) DRD1 promoter (e.g., SEQ ID NO: 716);
   (xiv) DRD2 promoter (e.g., SEQ ID NO: 717 or 718);
   (xv) C1QL2 promoter (e.g., SEQ ID NO: 719);
   (xvi) POMC promoter (e.g., SEQ ID NO: 720);
   (xvii) PROX1 promoter (e.g., SEQ ID NO: 721 or 722);
   (xviii) MAP1 B promoter (e.g., any one of SEQ ID NOs: 723-725);
   (xix) TUBA1A promoter (e.g., SEQ ID NO: 726 or 727);
   (xx) U6 promoter (e.g., any one of SEQ ID NOs: 728-733);
   (xxi) H1 promoter (e.g., SEQ ID NO: 734);
   (xxii) 7SK promoter (e.g., SEQ ID NO: 735);
   (xxiii) ApoE.hAAT promoter (e.g., SEQ ID NO: 736);
   (xxiv) CAG promoter (e.g., SEQ ID NO: 737);
   (xxv) CBA promoter (e.g., SEQ ID NO: 738);
   (xxvi) CK8 promoter (e.g., SEQ ID NO: 739);
   (xxvii) MU1A promoter (e.g., SEQ ID NO: 740);
   (xxviii) EF1-alpha promoter (e.g., SEQ ID NO: 741); and
   (xxix) TBG promoter (SEQ ID NO: 742);
(c) a stem-loop sequence containing:
   (i) a 5′ flanking sequence (e.g., any one of SEQ ID NOs: 752, 754, 756, 759, 762, 765, and 768);
   (ii) a 5p stem-loop arm containing a guide strand (e.g., any one of SEQ ID NOs: 1-100) or a passenger strand sequence (e.g., a sequence that is substantially or fully complementary to any one of SEQ ID NOs: 1-100);
   (iii) a microRNA loop sequence (e.g., any one of SEQ ID NOs: 758, 761, 764, 767, and 770); and
   (iv) a 3p stem-loop arm containing a passenger strand (e.g., a sequence that is substantially or fully complementary to any one of SEQ ID NOs: 1-100) or a guide strand sequence (e.g., any one of SEQ ID NOs: 1-100); and
   (v) a 3′ flanking sequence (e.g., any one of SEQ ID NOs: 753, 754, 757, 760, 763, 766, and 769);
(d) a 3′ untranslated region (UTR; e.g., any one of SEQ ID NOs: 750 and 751); and
(e) an AAV 3′ ITR sequence (e.g., any one of SEQ ID NOs: 748 and 749). An AAV vector containing a combination of any one of the above elements may be suitable for use according to the methods disclosed herein.

FIG. 31 is a bar graph showing total distance traveled by chronic epileptic mice during minutes of spontaneous exploration in an open field box before (open bars) and after (filled bars) treatment with 3.6E+9 MOI of a control vector (CV; n=3), 3.6E+9 MOI of construct SMSPV4 (n=4), 3.6E+8 MOI of construct SMSPV4 (n=4), 3.6E+9 MOI of construct SMSPVS (n=4), 3.6E+8 MOI of construct SMSPVS (n=4), 3.6E+9 MOI of construct SMSPV6 (n=4), and 3.6E+8 MOI of construct SMSPV6 (n=4). Treatment of mice with SMSPV4, SMSPVS, and SMSPV6, but not the control construct, reduced hyperlocomotion in mice, which is a proxy for epileptic behavior. Pre=pre-treatment; Post=post-treatment; E8=3.6E+8 MOI; E9=3.6E+9 MOI.

### DETAILED DESCRIPTION

Described herein are compositions and methods for the treatment of an epilepsy, such as, e.g., a temporal lobe epilepsy (TLE; e.g., TLE refractory to treatment) in a subject (such as a mammalian subject, for example, a human). For example, a therapeutically effective amount of an inhibitory RNA molecule (e.g., an antisense oligonucleotide (ASO) or nucleic acid vector encoding the same, such as those described herein) that targets an mRNA encoded by the glutamate ionotropic receptor kainate type subunit 2 (Grik2) gene can be administered, e.g., according to the methods described herein, to treat an epilepsy in a subject in need thereof. Described herein are compositions containing nucleic acid vectors (e.g., viral vectors, such as, e.g., lentiviral or adeno-associated viral (AAV) vectors) encoding an ASO agent targeting the Grik2 mRNA for the treatment of TLE.

### Grik2

Grik2 is a gene encoding an ionotropic glutamate receptor subunit, GluK2, that can be selectively activated by the agonist kainate. GluK2-containing kainate receptors (KARs), like other ionotropic glutamate receptors, exhibit fast ligand gating by glutamate, which acts by opening a cation channel pore permeable to sodium and potassium. KAR complexes can be assembled from several subunits as heteromeric or homomeric assemblies of KAR subunits. Such receptors feature an extracellular N-terminus and a large peptide loop that together form the ligand-binding domain and an intracellular C-terminus. The ionotropic glutamate receptor complex itself acts as a ligand-gated ion channel, and upon binding glutamate mediates the passage of charged ions across the neuronal membrane. Generally, KARs are multimeric assemblies of GluK1, 2 and/or 3 (previously named GluR5, GluR6 and GluR7, respectively), GluK4 (KA1) and GluK(KA2) subunits (Collingridge, Neuropharmacology. 2009 Jan;56(1):2-5). The various combinations of subunits involved in a KAR complex are often determined by RNA splicing and/or RNA editing (e.g., conversion of adenosine to inosine by adenosine deaminases) of mRNA encoding a particular KAR subunit. Furthermore, such RNA modification may impact the properties of the receptor, such as, e.g., altering calcium permeability of the channel. GluK2-containing KARs are suitable targets for modulation of ionotropic glutamate receptor activity and subsequently amelioration of symptoms related to epileptogenesis.

### Temporal Lobe Epilepsy

Epileptogenesis is a process that leads to the establishment of epilepsy and which may appear latent while cellular, molecular, and morphological changes leading to pathological neuronal network reorganization occur. TLE is characterized by two main types based on the anatomical origin of the epileptogenic focus. TLE originating from the mesial temporal lobe (e.g., hippocampus, parahippocampal gyrus, subiculum, and amygdala, among others) is named mesial TLE (mTLE), whereas TLE originating from the lateral temporal lobe (e.g., temporal neocortex) is referred to as lateral TLE (ITLE). Additional features characteristic of TLE may include neuronal cell death in the CA1, CA3, dentate hilus, and dentate gyrus (DG) regions of the hippocampus, reversal of the GABA reversal potential, granule cell (GC) dispersion in the DG, and sprouting of recurrent GC mossy fibers that leads to the formation of pathophysiological recurrent excitatory synapses onto dentate GCs (rMF-DGC synapses).

Various causal factors have been attributed to the etiology of TLE including mesial temporal sclerosis, traumatic brain injury, brain infections (e.g., encephalitis and meningitis), hypoxic brain injury, stroke, cerebral tumors, genetic syndromes, and febrile seizures. Because plasticity of the CNS depends on both the developmental state and brain region-specific susceptibility, not all subjects with brain injuries develop epilepsy. The hippocampus, including the DG, has been identified as a brain region particularly susceptible to damage that leads to TLE, and, in some instances, has been associated with treatment-resistant (i.e., refractory) epilepsy (Jarero-Basulto, J. J., et al. Pharmaceuticals, 2018, 11, 17; doi:10.3390/ph11010017). An amplification of excitatory glutamatergic signaling may facilitate spontaneous seizures (Kuruba, et al. Epilepsy Behay. 2009, 14 (Suppl. 1), 65-73). Chemical glutamate inhibitors, for example NMDA receptor antagonists, have been shown to block or reduce neuronal death by glutamate-mediated excitotoxicity and acute seizure generation. However, such agents exhibit poor efficacy in TLE (Foster, A C, and Kemp, J A. Curr. Opin. Pharmacol. 2006, 6, 7-17).

Without wishing to be bound by theory, aberrant rMF-DGC synapses, which operate via ectopic GluK2-containing KARs (Epsztein et al., 2005; Artinian et al., 2011, 2015) may play a key role in chronic seizures in TLE (Peret et al., 2014). For example, interictal spikes and ictal events (i.e., electrophysiological signatures of epileptiform brain activity) were reduced in transgenic mice lacking the GluK2 receptor subunit or in the presence of a pharmacological agent inhibiting GluK2/GluKreceptors (Peret et al., 2014; Crépel and Mulle, 2015). While knockdown or silencing of GluK2 in transgenic animal models designed to test these theories is feasible, designing an inhibitor selective for the GluK2 subunit and safe for use in humans is challenging. The GluK subunits are structurally conserved and their DNA coding sequences share significant homologies. The complex gene expression pattern in the brain with respect to homomeric and heteromeric ionotropic and metabotropic glutamate receptors further complicates any therapeutic strategy. The methods and compositions disclosed herein are suitable for the treatment of a TLE (e.g., mTLE or ITLE) by targeting Grik2 mRNA and decreasing (e.g., knocking down) the expression of GluK2-containing KARs in neurons or astroglia, which promotes, e.g., a reduction in spontaneous epileptiform discharges in neuronal circuits (e.g., hippocampal circuits). As such, the compositions and methods described herein target the physiological cause of the disease and can be used for curative therapy.

Oligonucleotide Agents Targeting Grik2 mRNA

Clinical management of TLE is notoriously difficult, with up to one third of TLE patients being unable to have adequate control of debilitating seizures using available medications. These patients often experience recurrent epileptic seizures that are refractory to treatment. In such scenarios, TLE patients may resort to invasive and irreversible surgical resection of the epileptogenic focus in the temporal lobe, which can result in unwanted cognitive deficits. Thus, a substantial fraction of TLE patients are in need of novel therapeutic avenues for treating pharmaco-resistant TLE. The compositions and methods described herein provide the benefit of treating the underlying molecular pathophysiology that leads to the development and progression of TLE.

The compositions described herein, which are polynucleotides encoding inhibitory RNA constructs (e.g., ASO agents or nucleic acid vectors encoding the same) that target a Grik2 mRNA (e.g., any one of SEQ ID NOs: 115-125), can be administered according to the methods described herein to treat TLE. The methods and compositions described herein can be used to treat a TLE patient having any type of TLE, such as, e.g., TLE with focal seizures, TLE with generalized seizures, mTLE, or ITLE. Furthermore, the presently disclosed methods and compositions may be used to treat TLE resulting from any etiology such as, e.g., mesial temporal sclerosis, traumatic brain injury, brain infections (e.g., encephalitis and meningitis), hypoxic brain injury, stroke, cerebral tumors, genetic syndromes, or febrile seizures. The compositions and methods described herein may also be administered as a preventative treatment to a subject at risk of developing TLE, e.g., a subject in the latent phase of TLE progression.

According to the methods and compositions disclosed herein, the ASO may inhibit the expression of the Grik2 mRNA by causing the degradation of the Grik2 mRNA in a cell (e.g., a neuron, such as, e.g., a hippocampal neuron, such as, e.g., a hippocampal neuron of the dentate gyrus, such as, e.g., a dentate granule cell (DGC)), thereby preventing translation of the mRNA into a functional GluK2 protein.

The ASO agents targeting the Grik2 mRNA disclosed herein may act to decrease the frequency of or completely inhibit the occurrence of epileptic brain activity (e.g., epileptiform discharges) in one or more brain regions. Such brain regions may include, but are not limited to the mesial temporal lobe, lateral temporal lobe, frontal lobe, or more specifically, hippocampus (e.g., DG, CA1, CA2, CA3, subiculum) or neocortex. Due to the aberrant expression of GluK2-containing KARs in rMF-DGCs of the DG, the occurrence of epileptic brain activity may be inhibited in the DG.

Accordingly, the present disclosure provides methods and compositions for reducing epileptiform discharges in a CNS cell (e.g., a DGC) by contacting the cell with an effective amount of an ASO having at least 85% (e.g., at least 86%, 90%, 95%, 96%, 97%, 98%, 99%, or more) sequence identity to any one of SEQ ID NOs: 1-100 or a nucleic acid vector encoding the same.

The ASO agent of the present disclosure may be a GluK2 inhibitor. In particular, the GluK2 inhibitor may be a Grik2 mRNA expression inhibitor. Inhibiting the expression of GluK2 may also inhibit the levels of GluK(Ruiz et al, J Neuroscience 2005). While not wishing to be bound to any theory, the disclosure is based on the principle that sufficient removal of GluK2 alone should remove all GluK2/GluK heteromers, since GluKsubunits alone are not capable of forming homomeric assemblies.

According to the disclosed methods and compositions, the ASO agents disclosed herein may have a length from to 50 nucleotides (e.g., 15, 16, 17, 18, 19, 20, 21, 22, 23, 24, or 25, 30, 35, 40, 45, or up to 50 nucleotides). For example, the ASO agent disclosed herein may have a length of 15 nucleotides. In another example, the ASO agent has a length of 16 nucleotides. In another example, the ASO agent has a length of 17 nucleotides. In another example, the ASO agent has a length of 18 nucleotides. In another example, the ASO agent has a length of 19 nucleotides. In another example, the ASO agent has a length of nucleotides. In another example, the ASO agent has a length of 21 nucleotides. In another example, the ASO agent has a length of 22 nucleotides. In another example, the ASO agent has a length of 23 nucleotides. In another example, the ASO agent has a length of 24 nucleotides. In another example, the ASO agent has a length of nucleotides. In another example, the ASO agent has a length of 25-nucleotides. In another example, the ASO agent has a length of 30-nucleotides. In another example, the ASO agent has a length of 35-nucleotides. In another example, the ASO agent has a length of 40-45 nucleotides. In another example, the ASO agent has a length of 45-50 nucleotides.

The ASO agents of the disclosure include a sequence that is at least substantially complementary or fully complementary to a region of the sequence of Grik2 mRNA (e.g., any one of SEQ ID NOs: 115-689) or variants thereof, said complementarity being sufficient to yield specific binding under intracellular conditions. For example, the present disclosure contemplates an ASO agent having an antisense sequence that is complementary to at least 7 (e.g., at least 7, 8, 9, 10, 11, 12, 13, 14, 15, 16, 17, 18, 19, 20, 21, 22, or more) consecutive nucleotides of one or more regions of a Grik2 mRNA. In a particular example, the ASO agent has an antisense sequence that is complementary to 7 consecutive nucleotides of one or more regions of a Grik2 mRNA. In another example, the ASO agent has an antisense sequence that is complementary to 8 consecutive nucleotides of one or more regions of a Grik2 mRNA. In another example, the ASO agent has an antisense sequence that is complementary to 9 consecutive nucleotides of one or more regions of a Grik2 mRNA. In another example, the ASO agent has an antisense sequence that is complementary to consecutive nucleotides of one or more regions of a Grik2 mRNA. In another example, the ASO agent has an antisense sequence that is complementary to 11 consecutive nucleotides of one or more regions of a Grik2 mRNA. In another example, the ASO agent has an antisense sequence that is complementary to 12 consecutive nucleotides of one or more regions of a Grik2 mRNA. In another example, the ASO agent has an antisense sequence that is complementary to 13 consecutive nucleotides of one or more regions of a Grik2 mRNA. In another example, the ASO agent has an antisense sequence that is complementary to 14 consecutive nucleotides of one or more regions of a Grik2 mRNA. In another example, the ASO agent has an antisense sequence that is complementary to consecutive nucleotides of one or more regions of a Grik2 mRNA. In another example, the ASO agent has an antisense sequence that is complementary to 16 consecutive nucleotides of one or more regions of a Grik2 mRNA. In another example, the ASO agent has an antisense sequence that is complementary to 17 consecutive nucleotides of one or more regions of a Grik2 mRNA. In another example, the ASO agent has an antisense sequence that is complementary to 18 consecutive nucleotides of one or more regions of a Grik2 mRNA. In another example, the ASO agent has an antisense sequence that is complementary to 19 consecutive nucleotides of one or more regions of a Grik2 mRNA. In another example, the ASO agent has an antisense sequence that is complementary to consecutive nucleotides of one or more regions of a Grik2 mRNA. In another example, the ASO agent has an antisense sequence that is complementary to 21 consecutive nucleotides of one or more regions of a Grik2 mRNA. In another example, the ASO agent has an antisense sequence that is complementary to 22 consecutive nucleotides of one or more regions of a Grik2 mRNA. In yet another example, the ASO agent has an antisense sequence that is 100% complementary to the nucleotides of one or more regions of a Grik2 mRNA.

The present disclosure contemplates ASO agents that, when bound to one or more regions of a Grik2 mRNA (e.g., any one of the regions of Grik2 mRNA described in SEQ ID NOs: 115-681), forms a duplex structure with the Grik2 mRNA of between 7-22 (e.g., 7, 8, 9, 10, 11, 12, 13, 14, 15, 16, 17, 18, 19, 20, 21, or 22) nucleotides in length. For example, the duplex structure between the ASO agent and the Grik2 mRNA may be 7 nucleotides in length. In another example, the duplex structure between the ASO agent and the Grik2 mRNA may be 8 nucleotides in length. In another example, the duplex structure between the ASO agent and the Grik2 mRNA may be 9 nucleotides in length. In another example, the duplex structure between the ASO agent and the Grik2 mRNA may be nucleotides in length. In another example, the duplex structure between the ASO agent and the Grik2 mRNA may be 11 nucleotides in length. In another example, the duplex structure between the ASO agent and the Grik2 mRNA may be 12 nucleotides in length. In another example, the duplex structure between the ASO agent and the Grik2 mRNA may be 13 nucleotides in length. In another example, the duplex structure between the ASO agent and the Grik2 mRNA may be 14 nucleotides in length. In another example, the duplex structure between the ASO agent and the Grik2 mRNA may be nucleotides in length. In another example, the duplex structure between the ASO agent and the Grik2 mRNA may be 16 nucleotides in length. In another example, the duplex structure between the ASO agent and the Grik2 mRNA may be 17 nucleotides in length. In another example, the duplex structure between the ASO agent and the Grik2 mRNA may be 18 nucleotides in length. In another example, the duplex structure between the ASO agent and the Grik2 mRNA may be 19 nucleotides in length. In another example, the duplex structure between the ASO agent and the Grik2 mRNA may be nucleotides in length. In another example, the duplex structure between the ASO agent and the Grik2 mRNA may be 21 nucleotides in length. In yet another example, the duplex structure between the ASO agent and the Grik2 mRNA may be nucleotides in length.

According to the disclosed methods and compositions, the duplex structure formed by an ASO agent (e.g., any one of the ASO agents disclosed herein, such as, e.g., any one of the ASO sequences of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100) and one or more regions of a Grik2 mRNA may include at least one (e.g., at least 1, 2, 3, 4, 5, 6, 7, 8, 9, 10, 11, 12, 13, 14, or 15) mismatch. For example, the duplex structure may contain 1 mismatch. In another example, the duplex structure contains 2 mismatches. In another example, the duplex structure contains 3 mismatches. In another example, the duplex structure contains 4 mismatches. In another example, the duplex structure contains mismatches. In another example, the duplex structure contains 6 mismatches. In another example, the duplex structure contains 7 mismatches. In another example, the duplex structure contains 8 mismatches. In another example, the duplex structure contains 9 mismatches. In another example, the duplex structure contains mismatches. In another example, the duplex structure contains 11 mismatches. In another example, the duplex structure contains 12 mismatches. In another example, the duplex structure contains 13 mismatches. In another example, the duplex structure contains 14 mismatches. In yet another example, the duplex structure contains mismatches.

Accordingly, an object of the present disclosure relates to isolated, synthetic, or recombinant ASO agents targeting Grik2 mRNA. The ASO agent of the disclosure may be of any suitable type, including RNA or DNA oligonucleotides. Thus, the disclosed methods and compositions feature a Grik2 expression inhibitor that is an ASO agent (e.g., siRNA, shRNA, miRNA, or shmiRNA, or shmiRNA). ASO agents, including antisense RNA molecules and antisense DNA molecules, may act to directly block the translation of Grik2 mRNA by binding thereto and preventing protein translation or increasing mRNA degradation, thereby decreasing the level and activity of GluK2 proteins. For example, ASO agents having at least about 19 bases and complementarity to unique regions of the mRNA transcript sequence encoding GluK2 can be synthesized, e.g., by conventional techniques (e.g., techniques disclosed herein) and administered by, e.g., intravenous injection or infusion, among other routes described herein, such as direct injection to a region of the brain. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732, each of which is incorporated by reference herein in its entirety).

In a particular example, a Grik2 ASO agent of the disclosure may be a short interfering RNA (siRNA). Grik2 gene expression can be reduced by contacting the subject or cell with a small double stranded RNA (dsRNA), or a vector encoding the same, thereby causing the production of a small double stranded RNA capable of specifically inhibiting Grik2 expression by degradation of mRNAs in a sequence-specific manner (e.g., by way of the RNA interference pathway). Methods for selecting an appropriate dsRNA or dsRNA-encoding vector are known in the art for genes whose sequence is known (e.g., see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836, each of which is incorporated by reference herein in its entirety).

The Grik2 ASO agent of the disclosure may also be a short hairpin RNA (shRNA). An shRNA is a sequence of RNA that makes a tight hairpin turn that can be used to silence gene expression via RNA interference. shRNA is generally expressed using a vector introduced into target cells, wherein the vector often utilizes the ubiquitous U6 promoter to ensure that the shRNA is constitutively expressed. This vector is usually passed on to daughter cells, allowing the gene silencing to be maintained following cell division. The shRNA hairpin structure is cleaved by the cellular machinery into siRNA, which is then bound to the RNA-induced silencing complex (RISC). This complex binds to and cleaves mRNAs that match the siRNA sequence to which it is bound.

Additionally, the Grik2 expression inhibitor of the disclosure may be a microRNA (miRNA). miRNA has a general meaning in the art and refers, e.g., to microRNA molecules that are generally 21 to 22 nucleotides in length, even though lengths of 19 and up to 23 nucleotides have been reported, and can be used to suppress translation of targeted mRNAs. miRNAs are each processed from a longer precursor RNA molecule (“precursor miRNA”). Precursor miRNAs are transcribed from non-protein-encoding genes. The precursor miRNAs have two regions of complementarity that allow them to form a stem-loop- or fold-back-like structure, which is cleaved in animals by a ribonuclease III-like nuclease enzyme called Dicer. The processed miRNA is typically a portion of the stem containing a “seed sequence” (typically 6-8 nucleotides) that is fully or substantially complementary to a region of the target mRNA. The processed miRNA (also referred to as “mature miRNA”) becomes part of a large complex to downregulate (e.g., decrease translation or degrade mRNA) of a particular target gene.

Furthermore, the GluK2 inhibitor of the disclosure is a miRNA-adapted shRNA (shmiRNA). shmiRNA agents refer to chimeric molecules that incorporate antisense sequences within the −5p or the −3p arm of a microRNA scaffold (e.g., a miR-scaffold) containing microRNA flanking and loop sequences. Compared to an shRNA, shmiRNA generally has a longer stem-loop structure based on microRNA-derived sequences, with the −5p and the −3p arm exhibiting full or substantial complementarity (e.g., mismatches, G:U wobbles). Owing to their longer sequences and processing requirements, shmiRNAs are generally expressed from a Pol II promoter. These constructs have also been shown to exhibit reduced toxicity as compared to shRNA-based agents.

Multiple miRNAs may be employed to knockdown Grik2 mRNA expression (and subsequently its gene product, GluK2). The miRNAs may be complementary to different target transcripts or different binding sites of a single target transcript. Multigene or multi-gene transcripts may also be utilized to enhance the efficiency of target gene knockdown. Multiple genes encoding the same miRNAs or different miRNAs may be regulated together in a single transcript, or as separate transcripts in a single vector cassette. miRNAs of the disclosure may be packaged into a vector, such as, e.g., a viral vector, including but not limited to recombinant adeno-associated viral (rAAV) vectors, lentiviral vectors, retroviral vectors and retrotransposon-based vector systems.

The ASO that is complementary (e.g., substantially or fully complementary) to the sense target sequence of a Grik2 mRNA is generally encoded by a DNA sequence for the production of any of the foregoing inhibitors (e.g., siRNAs, shRNAs, miRNAs, or shmiRNAs). The DNA encoding a double-stranded RNA of interest can be incorporated into a gene cassette (e.g., an expression cassette in which transcription of the DNA is controlled by a promoter).

### Antisense Oligonucleotide Sequences

According to the methods and compositions of the disclosure, the inhibitory RNA agents disclosed herein may include any one or more of the ASO agents disclosed in Table 2 (e.g., SEQ ID NOs: 1-100) or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding nucleic acid sequence of any one of SEQ ID NOs: 1-100, as is shown below. The ASO agent may bind to a corresponding target sequence of a Grik2 mRNA described in Table 4 below or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 below or any one of SEQ ID NOs: 164-681.

TABLE 2
Antisense oligonucleotide sequences targeting Grik2mRNA

| | | SEQ ID | cDNA sequence encoding target | SEQ ID | Target sequence nucleotide | | |
|---|---|---|---|---|---|---|---|
| ID | Guide sequence (ASO) | NO | sequence in human Grik2 mRNA | NO | position (Grik2 mRNA) | Grik2 Region | % KD |
| G0 | AAGGGUAGUAUUGGGUAGCA | 1 | TTGCTACCCAATACTACCCTT | 582 | 208-228 (SEQ ID NO: 116) | Exon 2 | 83% |
| | | | | | 501-521 (SEQ ID NO: 115) | Centroid Loop | |
| | | | | | | 1 | |
| TV | AGGCCCGAAGAUGGCAGCCA | 2 | GTGGCTGCCATCTTCGGGCCT | 583 | 307-327 (SEQ ID NO: 116) | Exon 3 | 0% |
| | | | | | 600-620 (SEQ ID NO: 115) | | |
| TU | AGCAUUGCAGAUGGACUGCA | 3 | GTGCAGTCCATCTGCAATGCT | 584 | 352-372 (SEQ ID NO: 116) | Exon 3 | 72% |
| | | | | | 645-665 (SEQ ID NO: 115) | | |
| TT | UGUUAACACCACUGUAUCGGU | 4 | ACCGATACAGTGGTGTTAACA | 585 | 806-826 (SEQ ID NO: 116) | Exon 6 | 83% |
| | | | | | 1099-1119 (SEQ ID NO: 115) | Centroid loop | |
| | | | | | | 2 | |
| G1 | AAUCGGGUUUCGGAGGUGCC | 5 | AGGCACCTCCGAAACCCGATT | 586 | 905-925 (SEQ ID NO: 116) | Exon 6 | 64% |
| | | | | | 1198-1218 (SEQ ID NO: 115) | Centroid loop | |
| | | | | | | 3 | |
| G2 | AUCGGGUUUCGGAGGUGCCU | 6 | CAGGCACCTCCGAAACCCGAT | 587 | 904-924 (SEQ ID NO: 116 | Exon 6 | 52% |
| | | | | | 1197-1217 (SEQ ID NO: 115) | Centroid loop | |
| | | | | | | 3 | |
| GD | UGUAGAUAACUCUCUGAGGA | 7 | CTCCTCAGAGAGTTATCTACA | 588 | 1396-1416 (SEQ ID NO: 116) | Exon 10 | 72% |
| | | | | | 1689-1709 (SEQ ID NO: 115) | Centroid Loop | |
| | | | | | | 5 | |
| G3 | UUAGUUCACGAACCAUUCCAU | 8 | ATGGAATGGTTCGTGAACTAA | 589 | 1496-1516 (SEQ ID NO: 116) | Exon 10 | 59% |
| | | | | | 1789-1809 (SEQ ID NO: 115) | Centroid Loop | |
| | | | | | | 6 | |
| G4 | AAUAACUGGGGCCUGUGGCU | 9 | AAGCCACAGGCCCCAGTTATT | 590 | 2632-2652 (SEQ ID NO: 116) | Exon 16 | 52% |
| | | | | | 2925-2945 (SEQ ID NO: 115) | Centroid Loop | |
| | | | | | | 8 | |
| TS | UUAGUGACACUUUUAUUGGU | 10 | AACCAATAAAAGTGTCACTAA | 591 | 3382-3402 (SEQ ID NO: 115) | 3′ UTR | 26% |
| | | | | | | Centroid Loop | |
| | | | | | | 11 | |
| | | | | | | Exon 16 | |
| TR | UUAUUAUUUGGGAUAUGGGG | 11 | CCCCCATATCCCAAATAATAA | 592 | 3792-3812 (SEQ ID NO: 115) | 3′ UTR | 71% |
| | | | | | | Centroid Loop | |
| | | | | | | 12 | |
| | | | | | | Exon 16 | |
| TQ | GAUGUUCGCUGGCUUUCCCU | 12 | AAGGGAAAGCCAGCGAACATC | 593 | 1252-1272 (SEQ ID NO: 116) | Exon 9 | 61% |
| | | | | | 1545-1565 (SEQ ID NO: 115) | | |
| TP | uGUUCCACUGUCAGAAAGGCG | 13 | CGCCTTTCTGACAGTGGAAC | 594 | 1968-1987 (SEQ ID NO: 116) | Exon 13 | 82% |
| | | | | | 2213-2233 (SEQ ID NO: 115) | Centroid | |
| | | | | | | Unpaired | |
| | | | | | | region 1 | |
| TO | CGUUCCACUGUCAGAAAGGC | 14 | CGCCTTTCTGACAGTGGAACG | 595 | 1968-1988 (SEQ ID NO: 116) | Exon 13 | 86% |
| | | | | | 2213-2233 (SEQ ID NO: 115) | Centroid | |
| | | | | | | Unpaired | |
| | | | | | | region 1 | |
| G5 | UGAUAUUCCAUCUCCUGGCAA | 15 | TTGCCAGGAGATGGAATATCA | 596 | 3347-3367 (SEQ ID NO: 115) | 3′ UTR | 89% |
| | | | | | | Centroid | |
| | | | | | | Unpaired | |
| | | | | | | region 3 | |
| | | | | | | Exon 16 | |
| TN | AUAAGUGAUGAACAUCUGCUU | 16 | AAGCAGATGTTCATCACTTAT | 597 | 3605-3625 (SEQ ID NO: 115) | 3′ UTR | 73% |
| | | | | | | Centroid | |
| | | | | | | Unpaired | |
| | | | | | | region 4 | |
| | | | | | | Exon 16 | |
| TM | UAUUUCCUUGAUAAUUGGCAU | 17 | ATGCCAATTATCAAGGAAATA | 598 | 3686-3706 (SEQ ID NO: 115) | 3′ UTR | 78% |
| G6 | uAUCCUCAAUUCUUCUACCAU | 18 | ATGGTAGAAGAATTGAGGAT | 599 | 2581-2601 (SEQ ID NO: 116) | Exon 16 | 76% |
| | | | | | 2874-2893 (SEQ ID NO: 115) | | |
| G7 | CAUCCUCAAUUCUUCUACCAU | 19 | ATGGTAGAAGAATTGAGGATG | 600 | 2581-2601 (SEQ ID NO: 116) | Exon 16 | 31% |
| | | | | | 2874-2893 (SEQ ID NO: 115) | | |
| TL | UGUCGAUUACACUGCAAGGAA | 20 | TTCCTTGCAGTGTAATCGACA | 601 | 1029-1049 (SEQ ID NO: 116) | Exon 7 | 85% |
| | | | | | 1322-1342 (SEQ ID NO: 115) | | |
| TJ | GAUGCGUCGAGUGGUGACCG | 21 | GCGGTCACCACTCGACGCATC | 602 | 197-217 (SEQ ID NO: 115) | 5′ UTR | 63% |
| | | | | | | Exon 1 | |
| TH | CUCGAACAUAGGUAAUAGCCA | 22 | TGGCTATTACCTATGTTCGAG | 603 | 1550-1570 (SEQ ID NO: 116) | Exon 11 | 70% |
| | | | | | 1843-1863 (SEQ ID NO: 115) | | |
| TG | GGUUCGGGUAGAAAUGAGGA | 23 | ATCCTCATTTCTACCCGAACC | 604 | 215-235 (SEQ ID NO: 115) | 5′ UTR | 14% |
| | | | | | | Exon 1 | |
| TF | uUAGCGUUCGGCUCCUGGGU | 24 | AACCCAGGAGCCGAACGCTA | 605 | 232-251 (SEQ ID NO: 115) | 5′ UTR | 49% |
| | | | | | | Exon 1 | |
| TE | CUAGCGUUCGGCUCCUGGGU | 25 | AACCCAGGAGCCGAACGCTAG | 606 | 232-252 (SEQ ID NO: 115) | 5′ UTR | 52% |
| | | | | | | Exon 1 | |
| TD | GUUCGGCUCCUGGGUUCGGG | 26 | ACCCGAACCCAGGAGCCGAAC | 607 | 227-247 (SEQ ID NO: 115) | 5′ UTR | 0% |
| | | | | | | Exon 1 | |
| GJ | AACGUUGGUGGUGCACACGC | 27 | TGCGTGTGCACCACCAACGTT | 608 | 4289-4309 (SEQ ID NO: 115) | 3′ UTR | 45% |
| | | | | | | Exon 16 | |
| TC | uGGUGCGCCUGAAGACUGGA | 28 | ATCCAGTCTTCAGGCGCACCA | 609 | 29-48 (SEQ ID NO: 116) | Exon 1 | 3% |
| | | | | | 322-341 (SEQ ID NO: 115) | Signal peptide | |
| CK | GCGCCUGAAGACUGGAU | 29 | ATCCAGTCTTCAGGCGCACCG | 610 | 29-49 (SEQ ID NO: 116) | Exon 1 | 6% |
| | | | | | 322-342 (SEQ ID NO: 115) | Signal peptide | |
| CL | uAGCGGGUCUGUAUGUGGGG | 30 | TCCCCACATACAGACCCGCT | 611 | 381-400 (SEQ ID NO: 116) | Exon 3 | 69% |
| | | | | | 674-693 (SEQ ID NO: 115) | | |
| CM | CAGCGGGUCUGUAUGUGGGG | 31 | TCCCCACATACAGACCCGCTG | 612 | 381-401 (SEQ ID NO: 116) | Exon 3 | 54% |
| | | | | | 674-694 (SEQ ID NO: 115) | | |
| CN | uACGCACUACCAUUCAUGCUU | 32 | AAGCATGAATGGTAGTGCGT | 613 | 4274-4293 (SEQ ID NO: 115) | 3′ UTR | 34% |
| | | | | | | Exon 16 | |
| CO | CACGCACUACCAUUCAUGCUU | 33 | AAGCATGAATGGTAGTGCGTG | 614 | 4274-4294 (SEQ ID NO: 115) | 3′ UTR | 0% |
| | | | | | | Exon 16 | |
| CP | uUCGAUGGUUGUUGACUCCAU | 34 | ATGGAGTCAACAACCATCGA | 615 | 2209-2228 (SEQ ID NO: 116) | Exon 14 | 66% |
| | | | | | 2502-2521 (SEQ ID NO: 115) | | |
| CQ | CUCGAUGGUUGUUGACUCCA | 35 | ATGGAGTCAACAACCATCGAG | 616 | 2209-2229 (SEQ ID NO: 116) | Exon 14 | 61% |
| | | | | | 2502-2522 (SEQ ID NO: 115) | | |
| CR | AGCGGGUCUGUAUGUGGGGA | 36 | TTCCCCACATACAGACCCGCT | 617 | 380-400 (SEQ ID NO: 116) | Exon 3 | 33% |
| | | | | | 673-693 (SEQ ID NO: 115) | | |
| CS | UUGAACGGCCACAGACACCAC | 37 | GTGGTGTCTGTGGCCGTTCAA | 618 | 985-1005 (SEQ ID NO: 116) | Exon 7 | 0% |
| | | | | | 1278-1298 (SEQ ID NO: 115) | | |
| CT | AAAGCGGGUCCCGAAGCGCC | 38 | TGGCGCTTCGGGACCCGCTTT | 619 | 1057-1077 (SEQ ID NO: 116) | Exon 7 | 2% |
| | | | | | 1350-1370 (SEQ ID NO: 115) | | |
| CU | AGACGCCUGGGUUUGUACCA | 39 | ATGGTACAAACCCAGGCGTCT | 620 | 1637-1657 (SEQ ID NO: 116) | Exon 11 | 42% |
| | | | | | 1930-1950 (SEQ ID NO: 115) | | |
| CV | AAACGAAUGAGACCAGUGCUG | 40 | CAGCACTGGTCTCATTCGTTT | 621 | 534-554 (SEQ ID NO: 116) | overlaps Exon | 43% |
| | | | | | 827-847 (SEQ ID NO: 115) | 3 and Exon 4 | |
| C | AUAAACGCAGUCCACUUCCAA | 41 | TTGGAAGTGGACTGCGTTTAT | 622 | 4078-4098 (SEQ ID NO: 115) | 3′ UTR | 80% |
| | | | | | | Exon 16 | |
| CX | GACCGCAGACACGAUCACGG | 42 | GCCGTGATCGTGTCTGCGGTC | 623 | 182-202 (SEQ ID NO: 115) | 5′ UTR | 65% |
| | | | | | | Exon 1 | |
| CY | UUCGGCUCCUGGGUUCGGGU | 43 | TACCCGAACCCAGGAGCCGAA | 624 | 226-246 (SEQ ID NO: 115) | 5′ UTR | 59% |
| | | | | | | Exon 1 | |
| CZ | UAAAGCGGGUCCCGAAGCGC | 44 | GGCGCTTCGGGACCCGCTTTA | 625 | 1058-1078 (SEQ ID NO: 116) | Exon 7 | 47% |
| | | | | | 1351-1371 (SEQ ID NO: 115) | | |
| D0 | uACGGCACCCACUUCCCCGAU | 45 | ATCGGGGAAGTGGGTGCCGT | 626 | 253-272 (SEQ ID NO: 115) | 5′ UTR | 28% |
| | | | | | | Exon 1 | |
| D1 | CACGGCACCCACUUCCCCGAU | 46 | ATCGGGGAAGTGGGTGCCGTG | 627 | 253-273 (SEQ ID NO: 115) | 5′ UTR | 29% |
| | | | | | | Exon 1 | |
| D2 | AGCGCCAGGGUUUAUGUCGA | 47 | ATCGACATAAACCCTGGCGCT | 628 | 1043-1063 (SEQ ID NO: 116) | Exon 7 | 50% |
| | | | | | 1336-1356 (SEQ ID NO: 115) | | |
| D3 | GUCUCCGCUUCCCAAACCCAU | 48 | ATGGGTTTGGGAAGCGGAGAC | 629 | 139-159 (SEQ ID NO: 115) | 5′ UTR | 28% |
| | | | | | | Exon 1 | |
| XS | GACGACAGUUUGUGCUUGGG | 49 | ACCCAAGCACAAACTGTCGTC | 630 | 3037-3057 (SEQ ID NO: 115) | 3′ UTR | 51% |
| | | | | | | Exon 16 | |
| XT | AGCCUCGUGGAAACCAGGGG | 50 | ACCCCTGGTTTCCACGAGGCT | 631 | 4417-4437 (SEQ ID NO: 115) | 3′ UTR | 57% |
| | | | | | | Exon 16 | |
| XU | UGUCUCGAUAUGGAGAACCCA | 51 | TGGGTTCTCCATATCGAGACA | 632 | 2309-2329 (SEQ ID NO: 116) | Exon 15 | 68% |
| | | | | | 2602-2622 (SEQ ID NO: 115) | | |
| XV | uGACGCUGGCACUUCAGGGA | 52 | GTCCCTGAAGTGCCAGCGTCA | 633 | 2601-2620 (SEQ ID NO: 116) | Exon 16 | 0% |
| | | | | | 2894-2913 (SEQ ID NO: 115) | 3′ end of CDS | |
| XW | CGACGCUGGCACUUCAGGGA | 53 | GTCCCTGAAGTGCCAGCGTCG | 634 | 2601-2621 (SEQ ID NO: 116) | Exon 16 | 9% |
| | | | | | 2894-2914 (SEQ ID NO: 115) | 3′ end of CDS | |
| XZ | AGACACGAUCACGGCAUGGU | 54 | GACCATGCCGTGATCGTGTCT | 635 | 176-196 (SEQ ID NO: 115) | 5′ UTR | 12% |
| | | | | | | Exon 1 | |
| Y0 | UUCCCCGAUCUAGCGUUCGG | 55 | GCCGAACGCTAGATCGGGGAA | 636 | 241-261 (SEQ ID NO: 115) | 5′ UTR | 6.84% |
| | | | | | | Exon 1 | |
| Y1 | UGCAAUCGUUCCAUCGACCAC | 56 | GTGGTCGATGGAACGATTGCA | 637 | 885-905 (SEQ ID NO: 116) | Exon 6 | 40% |
| | | | | | 1178-1198 (SEQ ID NO: 115) | | |
| Y2 | uUGAAUCGGGUUUCGGAGGU | 57 | CACCTCCGAAACCCGATTCA | 638 | 908-927 (SEQ ID NO: 116) | Exon 6 | 39% |
| | | | | | 1201-1220 (SEQ ID NO: 115) | | |
| Y3 | CUGAAUCGGGUUUCGGAGGU | 58 | CACCTCCGAAACCCGATTCAG | 639 | 908-928 (SEQ ID NO: 116) | Exon 6 | 22% |
| | | | | | 1201-1221 (SEQ ID NO: 115) | | |
| Y4 | AAGGCCCGAAGAUGGCAGCC | 59 | TGGCTGCCATCTTCGGGCCTT | 640 | 308-328 (SEQ ID NO: 116) | Exon 3 | 0% |
| | | | | | 601-621 (SEQ ID NO: 115) | | |
| Y5 | UUGAGUCGAAGAUUAUACCUU | 60 | AAGGTATAATCTTCGACTCAA | 641 | 579-599 (SEQ ID NO: 116) | Exon 4 | 81% |
| | | | | | 872-892 (SEQ ID NO: 115) | | |
| Y6 | GGCUAGUAACAUCAUCACCUC | 61 | GAGGTGATGATGTTACTAGCC | 642 | 3479-3499 (SEQ ID NO: 115) | 3′ UTR | 41% |
| | | | | | | Exon 16 | |
| Y7 | AGAUAUCAGGGGAGAGAGGA | 62 | ATCCTCTCTCCCCTGATATCT | 643 | 1670-1690 (SEQ ID NO: 116) | Exon 11 | 50% |
| | | | | | 1963-1983 (SEQ ID NO: 115) | | |
| YA | GGUUGCAUAUUUCCACAGGAA | 63 | TTCCTGTGGAAATATGCAACC | 644 | 3085-3105 (SEQ ID NO: 115) | 3′ UTR | 67% |
| | | | | | | Exon 16 | |
| | | | | | | Centroid loop | |
| | | | | | | 9 | |
| GF | UGCGUCGAGUGGUGACCGCA | 64 | CTGCGGTCACCACTCGACGCA | 645 | 195-215 (SEQ ID NO: 115) | 5′ UTR | 63% |
| | | | | | | Exon 1 | |
| GE | UUAGUCGGAGAGCAUCCGGG | 65 | TCCCGGATGCTCTCCGACTAA | 646 | 42-62 (SEQ ID NO: 115) | 5′ UTR | 76% |
| | | | | | | Exon 1 | |
| GH | AUGCGUCGAGUGGUGACCGC | 66 | TGCGGTCACCACTCGACGCAT | 647 | 196-216 (SEQ ID NO: 115) | 5′ UTR | 21% |
| | | | | | | Exon 1 | |
| YB | uAUCCGGGAGAAAUCCAGCAC | 67 | GTGCTGGATTTCTCCCGGAT | 648 | 30-49 (SEQ ID NO: 115) | 5′ UTR | 56% |
| | | | | | | Exon 1 | |
| G9 | CCCAUAGCUAAUGCCUGUUUU | 68 | AAAACAGGCATTAGCTATGGG | 649 | 717-737 (SEQ ID NO: 116) | Overlaps Exon | 77% |
| | | | | | 1010-1030 (SEQ ID NO: 115) | 4 and Exon 5 | |
| ME | uUGUCAUCAUUCCCAUAGCUA | 69 | TAGCTATGGGAATGATGACAA | 650 | 728-747 (SEQ ID NO: 116) | Exon 5 | 77% |
| | | | | | 1021-1040 (SEQ ID NO: 115) | | |
| MD | CAUUCCCAUAGCUAAUGCCUG | 70 | CAGGCATTAGCTATGGGAATG | 651 | 721-741 (SEQ ID NO: 116) | Overlaps Exon | 0% |
| | | | | | 1014-1034 (SEQ ID NO: 115) | 4 and Exon 5 | |
| GB | CCCAUAGCUAAUGCCUGCUUU | 71 | AAAGCAGGCATTAGCTATGG | 652 | (MOUSE) | | 0% |
| MR | AAACCACCAAAUGCCUCCCAC | 72 | GTGGGAGGCATTTGGTGGTTT | 653 | 1906-1926 (SEQ ID NO: 116) | Exon 13 | 39% |
| | | | | | 2199-2219 (SEQ ID NO: 115) | Centroid | |
| | | | | | | Unpaired | |
| | | | | | | region 1 | |
| MQ | AUGAUAAGUGUGAAAAACCAC | 73 | GTGGTTTTTCACACTTATCAT | 654 | 1920-1940 (SEQ ID NO: 116) | Exon | 59% |
| | | | | | 2213-2233 (SEQ ID NO: 115) | 13Centroid | |
| | | | | | | Unpaired | |
| | | | | | | region 1 | |
| TK | AGUCGAUGACCUUCUCUCGAA | 74 | TTCGAGAGAAGGTCATCGACT | 655 | 1565-1585 (SEQ ID NO: 116) | Exon 11 | 85% |
| | | | | | 1858-1878 (SEQ ID NO: 115) | | |
| TI | uUCGAACAUAGGUAAUAGCCA | 75 | TGGCTATTACCTATGTTCGA | 656 | 1550-1569 (SEQ ID NO: 116) | Exon 11 | 80% |
| | | | | | 1843-1862 (SEQ ID NO: 115) | | |
| MP | GACACCUGGUGCUUCCAGCG | 76 | CCGCTGGAAGCACCAGGTGTC | 657 | 396-416 (SEQ ID NO: 116) | Exon 3 | 14% |
| | | | | | 689-709 (SEQ ID NO: 115) | | |
| GI | GUCUCGAUAUGGAGAACCCAU | 77 | ATGGGTTCTCCATATCGAGAC | 658 | 2308-2328 (SEQ ID NO: 116) | Overlaps with | 83% |
| | | | | | 2601-2621 (SEQ ID NO: 115) | Exon 14 and | |
| | | | | | | 15 | |
| MO | uGAUAUGGAGAACCCAUGGGA | 78 | TCCCATGGGTTCTCCATATCA | 659 | 2304-2323 (SEQ ID NO: 116) | Overlaps with | 43% |
| | | | | | 2597-2616 (SEQ ID NO: 115) | Exon 14 and | |
| | | | | | | 15 | |
| MN | GAUAUGGAGAACCCAUGGGA | 79 | CTCCCATGGGTTCTCCATATC | 660 | 2303-2323 (SEQ ID NO: 116) | Exon 14 | 51% |
| | | | | | 2596-2616 (SEQ ID NO: 115) | | |
| M | CAGAGCAUUGCAGAUGGACU | 80 | CAGTCCATCTGCAATGCTCTG | 661 | 355-375 (SEQ ID NO: 116) | Exon 3 | 79% |
| | | | | | 648-668 (SEQ ID NO: 115) | | |
| MV | CCCAGAGCAUUGCAGAUGGA | 81 | GTCCATCTGCAATGCTCTGGG | 662 | 357-377 (SEQ ID NO: 116) | Exon 3 | 31% |
| | | | | | 650-670 (SEQ ID NO: 115) | | |
| XX | uACCACGUCUGAGUCAGGGUU | 82 | AACCCTGACTCAGACGTGGT | 663 | 1786-1805 (SEQ ID NO: 116) | Exon 12 | 67% |
| | | | | | 2079-2098 (SEQ ID NO: 115) | | |
| XY | CACCACGUCUGAGUCAGGGU | 83 | AACCCTGACTCAGACGTGGTG | 664 | 1786-1806 (SEQ ID NO: 116) | Exon 12 | 67% |
| | | | | | 2079-2099 (SEQ ID NO: 115) | | |
| M | uUGAGUCAGGGUUGCAAGGG | 84 | ACCCTTGCAACCCTGACTCAG | 665 | 1778-1797 (SEQ ID NO: 116) | Exon 12 | 62% |
| | | | | | 2071-2090 (SEQ ID NO: 115) | | |
| ML | AGAGCUCCAACUCCAAACCAG | 85 | CTGGTTTGGAGTTGGAGCTCT | 666 | 1836-1856 (SEQ ID NO: 116) | Exon 12 | 80% |
| | | | | | 2129-2149 (SEQ ID NO: 115) | | |
| MK | UGAUGGAGCUUUGAUGAGCU | 86 | GAGCTCATCAAAGCTCCATCA | 667 | 559-579 (SEQ ID NO: 116) | Exon 4 | 26% |
| | | | | | 852-872 (SEQ ID NO: 115) | | |
| Y8 | uAUAGGUAAUAGCCAGUGGAG | 87 | CTCCACTGGCTATTACCTAT | 668 | 1544-1563 (SEQ ID NO: 116) | Exon 11 | 80% |
| | | | | | 1837-1856 (SEQ ID NO: 115) | | |
| Y9 | CAUAGGUAAUAGCCAGUGGA | 88 | CTCCACTGGCTATTACCTATG | 669 | 1544-1564 (SEQ ID NO: 116) | Exon 11 | 87% |
| | | | | | 1837-1857 (SEQ ID NO: 115) | | |
| MJ | GAGCAACUGCAAGGUCAGCU | 89 | AAGCTGACCTTGCAGTTGCTC | 670 | 1526-1546 (SEQ ID NO: 116) | Exon 11 | 26% |
| MI | AGGUAAUAGCCAGUGGAGCAA | 90 | TTGCTCCACTGGCTATTACCT | 671 | 1541-1561 (SEQ ID NO: 116) | Exon 11 | 55% |
| | | | | | 1834-1854 (SEQ ID NO: 115) | | |
| GG | AGUCGUCAUAAAUCCAUCCAG | 91 | CTGGATGGATTTATGACGACT | 672 | 934-954 (SEQ ID NO: 116) | Exon 6 | 67% |
| | | | | | 1227-1247 (SEQ ID NO: 115) | | |
| G8 | ACUGACAUAGAAGGAAUCUUU | 92 | AAAGATTCCTTCTATGTCAGT | 673 | 424-444 (SEQ ID NO: 116) | Exon 3 | 66% |
| | | | | | 717-737 (SEQ ID NO: 115) | | |
| MF | UAGAGACUGACAUAGAAGGAA | 93 | TTCCTTCTATGTCAGTCTCTA | 674 | 429-449 (SEQ ID NO: 116) | Exon 3 | 69% |
| | | | | | 722-742 (SEQ ID NO: 115) | | |
| MH | uGUCAUAAAUCCAUCCAGCAA | 94 | TTGCTGGATGGATTTATGACA | 675 | 931-950 (SEQ ID NO: 116) | Exon 6 | 74% |
| | | | | | 1224-1243 (SEQ ID NO: 115) | | |
| MG | uAUCAGUCGUCAUAAAUCCAU | 95 | ATGGATTTATGACGACTGATA | 676 | 938-957 (SEQ ID NO: 116) | Exon 6 | 71% |
| | | | | | 1231-1250 (SEQ ID NO: 115) | | |
| MU | UUCAUAUGUAAAGCCAAGGAU | 96 | ATCCTTGGCTTTACATATGAA | 677 | 1417-1437 (SEQ ID NO: 116) | Exon 10 | 82% |
| | | | | | 1710-1730 (SEQ ID NO: 115) | Centroid Loop | |
| | | | | | | 5 | |
| GA | ACUGACAUAGAAGGAAUCCUU | 97 | AAGGATTCCTTCTATGTCAGT | 678 | (MOUSE) | 3′ UTR | 28% |
| MT | AAUGGACAAUGGAAUGGAAUG | 98 | CATTCCATTCCATTGTCCATT | 679 | 1483-1503 (SEQ ID NO: 116) | Exon 10 | 4% |
| | | | | | 1776-1796 (SEQ ID NO: 115) | Centroid Loop | |
| | | | | | | 6 | |
| MS | AUGGAAUGGAAUGGUUCGUG | 99 | TCACGAACCATTCCATTCCAT | 680 | 1491-1511 (SEQ ID NO: 116) | Exon 10 | 0% |
| | | | | | 1784-1804 (SEQ ID NO: 115) | Centroid Loop | |
| | | | | | | 6 | |
| ZZ | UAGUCGGAGAGCAUCCGGGA | 100 | CTCCCGGATGCTCTCCGACTA | 681 | 41-61 (SEQ ID NO: 115) | 5′ UTR | N/A |
| | | | | | | Exon 1 | |

Note:
% KD values correspond to a percent knockdown of GluK2 protein achieved by the identified ASO agent in a dual-luciferase reporter assay, as is described in detail in Example 1 A.

The foregoing sequences are represented as DNA (i.e., cDNA) sequences that can be incorporated into a vector of the disclosure; however, these sequences may also be represented as corresponding RNA sequences that are synthesized from the vector within the cell. One skilled in the art would understand that the cDNA sequence is equivalent to the mRNA sequence, except for the substitution of uridines with thymidines, and can be used for the same purpose herein, i.e., the generation of an antisense oligonucleotide for inhibiting the expression of Grik2 mRNA. In the case of DNA vectors (e.g., AAV), the polynucleotide containing the antisense nucleic acid is a DNA sequence. In the case of RNA vectors, the transgene cassette incorporates the RNA equivalent of the antisense DNA sequences described herein.

An ASO sequence of the present disclosure may have at least 85% (e.g., at least 86%, 90%, 95%, 96%, 97%, 98%, 99%, or more) sequence identity to the nucleic acid sequence of SEQ ID NO: 1. For example, the ASO may have at least 90% (e.g., at least 90%, 95%, 96%, 97%, 98%, 99%, or more) sequence identity to the nucleic acid sequence of SEQ ID NO: 1. In another example, the ASO may have at least 95% (e.g., at least 95%, 96%, 97%, 98%, 99%, or more) sequence identity to the nucleic acid sequence of SEQ ID NO: 1. In a further example, the ASO may have the nucleic acid sequence of SEQ ID NO: 1.

An ASO sequence of the present disclosure may have at least 85% (e.g., at least 86%, 90%, 95%, 96%, 97%, 98%, 99%, or more) sequence identity to the nucleic acid sequence of SEQ ID NO: 2. For example, the ASO may have at least 90% (e.g., at least 90%, 95%, 96%, 97%, 98%, 99%, or more) sequence identity to the nucleic acid sequence of SEQ ID NO: 2. In another example, the ASO may have at least 95% (e.g., at least 95%, 96%, 97%, 98%, 99%, or more) sequence identity to the nucleic acid sequence of SEQ ID NO: 2. In a further example, the ASO may have the nucleic acid sequence of SEQ ID NO: 2.

An ASO sequence of the present disclosure may have at least 85% (e.g., at least 86%, 90%, 95%, 96%, 97%, 98%, 99%, or more) sequence identity to the nucleic acid sequence of SEQ ID NO: 3. For example, the ASO may have at least 90% (e.g., at least 90%, 95%, 96%, 97%, 98%, 99%, or more) sequence identity to the nucleic acid sequence of SEQ ID NO: 3. In another example, the ASO may have at least 95% (e.g., at least 95%, 96%, 97%, 98%, 99%, or more) sequence identity to the nucleic acid sequence of SEQ ID NO: 3. In a further example, the ASO may have the nucleic acid sequence of SEQ ID NO: 3.

An ASO sequence of the present disclosure may have at least 85% (e.g., at least 86%, 90%, 95%, 96%, 97%, 98%, 99%, or more) sequence identity to the nucleic acid sequence of SEQ ID NO: 4. For example, the ASO may have at least 90% (e.g., at least 90%, 95%, 96%, 97%, 98%, 99%, or more) sequence identity to the nucleic acid sequence of SEQ ID NO: 4. In another example, the ASO may have at least 95% (e.g., at least 95%, 96%, 97%, 98%, 99%, or more) sequence identity to the nucleic acid sequence of SEQ ID NO: 4. In a further example, the ASO may have the nucleic acid sequence of SEQ ID NO: 4.

An ASO sequence of the present disclosure may have at least 85% (e.g., at least 86%, 90%, 95%, 96%, 97%, 98%, 99%, or more) sequence identity to the nucleic acid sequence of SEQ ID NO: 5. For example, the ASO may have at least 90% (e.g., at least 90%, 95%, 96%, 97%, 98%, 99%, or more) sequence identity to the nucleic acid sequence of SEQ ID NO: 5. In another example, the ASO may have at least 95% (e.g., at least 95%, 96%, 97%, 98%, 99%, or more) sequence identity to the nucleic acid sequence of SEQ ID NO: 5. In a further example, the ASO may have the nucleic acid sequence of SEQ ID NO: 5.

An ASO sequence of the present disclosure may have at least 85% (e.g., at least 86%, 90%, 95%, 96%, 97%, 98%, 99%, or more) sequence identity to the nucleic acid sequence of SEQ ID NO: 6. For example, the ASO may have at least 90% (e.g., at least 90%, 95%, 96%, 97%, 98%, 99%, or more) sequence identity to the nucleic acid sequence of SEQ ID NO: 6. In another example, the ASO may have at least 95% (e.g., at least 95%, 96%, 97%, 98%, 99%, or more) sequence identity to the nucleic acid sequence of SEQ ID NO: 6. In a further example, the ASO may have the nucleic acid sequence of SEQ ID NO: 6.

An ASO sequence of the present disclosure may have at least 85% (e.g., at least 86%, 90%, 95%, 96%, 97%, 98%, 99%, or more) sequence identity to the nucleic acid sequence of SEQ ID NO: 7. For example, the ASO may have at least 90% (e.g., at least 90%, 95%, 96%, 97%, 98%, 99%, or more) sequence identity to the nucleic acid sequence of SEQ ID NO: 7. In another example, the ASO may have at least 95% (e.g., at least 95%, 96%, 97%, 98%, 99%, or more) sequence identity to the nucleic acid sequence of SEQ ID NO: 7. In a further example, the ASO may have the nucleic acid sequence of SEQ ID NO: 7.

An ASO sequence of the present disclosure may have at least 85% (e.g., at least 86%, 90%, 95%, 96%, 97%, 98%, 99%, or more) sequence identity to the nucleic acid sequence of SEQ ID NO: 8. For example, the ASO may have at least 90% (e.g., at least 90%, 95%, 96%, 97%, 98%, 99%, or more) sequence identity to the nucleic acid sequence of SEQ ID NO: 8. In another example, the ASO may have at least 95% (e.g., at least 95%, 96%, 97%, 98%, 99%, or more) sequence identity to the nucleic acid sequence of SEQ ID NO: 8. In a further example, the ASO may have the nucleic acid sequence of SEQ ID NO: 8.

An ASO sequence of the present disclosure may have at least 85% (e.g., at least 86%, 90%, 95%, 25 96%, 97%, 98%, 99%, or more) sequence identity to the nucleic acid sequence of SEQ ID NO: 9. For example, the ASO may have at least 90% (e.g., at least 90%, 95%, 96%, 97%, 98%, 99%, or more) sequence identity to the nucleic acid sequence of SEQ ID NO: 9. In another example, the ASO may have at least 95% (e.g., at least 95%, 96%, 97%, 98%, 99%, or more) sequence identity to the nucleic acid sequence of SEQ ID NO: 9. In a further example, the ASO may have the nucleic acid sequence of SEQ ID NO: 9.

An ASO sequence of the present disclosure may have at least 85% (e.g., at least 86%, 90%, 95%, 96%, 97%, 98%, 99%, or more) sequence identity to the nucleic acid sequence of SEQ ID NO: 10. For example, the ASO may have at least 90% (e.g., at least 90%, 95%, 96%, 97%, 98%, 99%, or more) sequence identity to the nucleic acid sequence of SEQ ID NO: 10. In another example, the ASO may have at least 95% (e.g., at least 95%, 96%, 97%, 98%, 99%, or more) sequence identity to the nucleic acid sequence of SEQ ID NO: 10. In a further example, the ASO may have the nucleic acid sequence of SEQ ID NO: 10.

An ASO sequence of the present disclosure may have at least 85% (e.g., at least 86%, 90%, 95%, 96%, 97%, 98%, 99%, or more) sequence identity to the nucleic acid sequence of SEQ ID NO: 11. For example, the ASO may have at least 90% (e.g., at least 90%, 95%, 96%, 97%, 98%, 99%, or more) sequence identity to the nucleic acid sequence of SEQ ID NO: 11. In another example, the ASO may have at least 95% (e.g., at least 95%, 96%, 97%, 98%, 99%, or more) sequence identity to the nucleic acid sequence of SEQ ID NO: 11. In a further example, the ASO may have the nucleic acid sequence of SEQ ID NO: 11.

An ASO sequence of the present disclosure may have at least 85% (e.g., at least 86%, 90%, 95%, 96%, 97%, 98%, 99%, or more) sequence identity to the nucleic acid sequence of SEQ ID NO: 12. For example, the ASO may have at least 90% (e.g., at least 90%, 95%, 96%, 97%, 98%, 99%, or more) sequence identity to the nucleic acid sequence of SEQ ID NO: 12. In another example, the ASO may have at least 95% (e.g., at least 95%, 96%, 97%, 98%, 99%, or more) sequence identity to the nucleic acid sequence of SEQ ID NO: 12. In a further example, the ASO may have the nucleic acid sequence of SEQ ID NO: 12.

An ASO sequence of the present disclosure may have at least 85% (e.g., at least 86%, 90%, 95%, 96%, 97%, 98%, 99%, or more) sequence identity to the nucleic acid sequence of SEQ ID NO: 13. For example, the ASO may have at least 90% (e.g., at least 90%, 95%, 96%, 97%, 98%, 99%, or more) sequence identity to the nucleic acid sequence of SEQ ID NO: 13. In another example, the ASO may have at least 95% (e.g., at least 95%, 96%, 97%, 98%, 99%, or more) sequence identity to the nucleic acid sequence of SEQ ID NO: 13. In a further example, the ASO may have the nucleic acid sequence of SEQ ID NO: 13.

An ASO sequence of the present disclosure may have at least 85% (e.g., at least 86%, 90%, 95%, 96%, 97%, 98%, 99%, or more) sequence identity to the nucleic acid sequence of SEQ ID NO: 14. For example, the ASO may have at least 90% (e.g., at least 90%, 95%, 96%, 97%, 98%, 99%, or more) sequence identity to the nucleic acid sequence of SEQ ID NO: 14. In another example, the ASO may have at least 95% (e.g., at least 95%, 96%, 97%, 98%, 99%, or more) sequence identity to the nucleic acid sequence of SEQ ID NO: 14. In a further example, the ASO may have the nucleic acid sequence of SEQ ID NO: 14.

An ASO sequence of the present disclosure may have at least 85% (e.g., at least 86%, 90%, 95%, 96%, 97%, 98%, 99%, or more) sequence identity to the nucleic acid sequence of SEQ ID NO: 15. For example, the ASO may have at least 90% (e.g., at least 90%, 95%, 96%, 97%, 98%, 99%, or more) sequence identity to the nucleic acid sequence of SEQ ID NO: 15. In another example, the ASO may have at least 95% (e.g., at least 95%, 96%, 97%, 98%, 99%, or more) sequence identity to the nucleic acid sequence of SEQ ID NO: 15. In a further example, the ASO may have the nucleic acid sequence of SEQ ID NO: 15.

An ASO sequence of the present disclosure may have at least 85% (e.g., at least 86%, 90%, 95%, 96%, 97%, 98%, 99%, or more) sequence identity to the nucleic acid sequence of SEQ ID NO: 16. For example, the ASO may have at least 90% (e.g., at least 90%, 95%, 96%, 97%, 98%, 99%, or more) sequence identity to the nucleic acid sequence of SEQ ID NO: 16. In another example, the ASO may have at least 95% (e.g., at least 95%, 96%, 97%, 98%, 99%, or more) sequence identity to the nucleic acid sequence of SEQ ID NO: 16. In a further example, the ASO may have the nucleic acid sequence of SEQ ID NO: 16.

An ASO sequence of the present disclosure may have at least 85% (e.g., at least 86%, 90%, 95%, 96%, 97%, 98%, 99%, or more) sequence identity to the nucleic acid sequence of SEQ ID NO: 17. For example, the ASO may have at least 90% (e.g., at least 90%, 95%, 96%, 97%, 98%, 99%, or more) sequence identity to the nucleic acid sequence of SEQ ID NO: 17. In another example, the ASO may have at least 95% (e.g., at least 95%, 96%, 97%, 98%, 99%, or more) sequence identity to the nucleic acid sequence of SEQ ID NO: 17. In a further example, the ASO may have the nucleic acid sequence of SEQ ID NO: 17.

An ASO sequence of the present disclosure may have at least 85% (e.g., at least 86%, 90%, 95%, 96%, 97%, 98%, 99%, or more) sequence identity to the nucleic acid sequence of SEQ ID NO: 18. For example, the ASO may have at least 90% (e.g., at least 90%, 95%, 96%, 97%, 98%, 99%, or more) sequence identity to the nucleic acid sequence of SEQ ID NO: 18. In another example, the ASO may have at least 95% (e.g., at least 95%, 96%, 97%, 98%, 99%, or more) sequence identity to the nucleic acid sequence of SEQ ID NO: 18. In a further example, the ASO may have the nucleic acid sequence of SEQ ID NO: 18.

An ASO sequence of the present disclosure may have at least 85% (e.g., at least 86%, 90%, 95%, 96%, 97%, 98%, 99%, or more) sequence identity to the nucleic acid sequence of SEQ ID NO: 19. For example, the ASO may have at least 90% (e.g., at least 90%, 95%, 96%, 97%, 98%, 99%, or more) sequence identity to the nucleic acid sequence of SEQ ID NO: 19. In another example, the ASO may have at least 95% (e.g., at least 95%, 96%, 97%, 98%, 99%, or more) sequence identity to the nucleic acid sequence of SEQ ID NO: 19. In a further example, the ASO may have the nucleic acid sequence of SEQ ID NO: 19.

An ASO sequence of the present disclosure may have at least 85% (e.g., at least 86%, 90%, 95%, 96%, 97%, 98%, 99%, or more) sequence identity to the nucleic acid sequence of SEQ ID NO: 20. For example, the ASO may have at least 90% (e.g., at least 90%, 95%, 96%, 97%, 98%, 99%, or more) sequence identity to the nucleic acid sequence of SEQ ID NO: 20. In another example, the ASO may have at least 95% (e.g., at least 95%, 96%, 97%, 98%, 99%, or more) sequence identity to the nucleic acid sequence of SEQ ID NO: 20. In a further example, the ASO may have the nucleic acid sequence of SEQ ID NO: 20.

An ASO sequence of the present disclosure may have at least 85% (e.g., at least 86%, 90%, 95%, 96%, 97%, 98%, 99%, or more) sequence identity to the nucleic acid sequence of SEQ ID NO: 21. For example, the ASO may have at least 90% (e.g., at least 90%, 95%, 96%, 97%, 98%, 99%, or more) sequence identity to the nucleic acid sequence of SEQ ID NO: 21. In another example, the ASO may have at least 95% (e.g., at least 95%, 96%, 97%, 98%, 99%, or more) sequence identity to the nucleic acid sequence of SEQ ID NO: 21. In a further example, the ASO may have the nucleic acid sequence of SEQ ID NO: 21.

An ASO sequence of the present disclosure may have at least 85% (e.g., at least 86%, 90%, 95%, 96%, 97%, 98%, 99%, or more) sequence identity to the nucleic acid sequence of SEQ ID NO: 22. For example, the ASO may have at least 90% (e.g., at least 90%, 95%, 96%, 97%, 98%, 99%, or more) sequence identity to the nucleic acid sequence of SEQ ID NO: 22. In another example, the ASO may have at least 95% (e.g., at least 95%, 96%, 97%, 98%, 99%, or more) sequence identity to the nucleic acid sequence of SEQ ID NO: 22. In a further example, the ASO may have the nucleic acid sequence of SEQ ID NO: 22.

An ASO sequence of the present disclosure may have at least 85% (e.g., at least 86%, 90%, 95%, 96%, 97%, 98%, 99%, or more) sequence identity to the nucleic acid sequence of SEQ ID NO: 23. For example, the ASO may have at least 90% (e.g., at least 90%, 95%, 96%, 97%, 98%, 99%, or more) sequence identity to the nucleic acid sequence of SEQ ID NO: 23. In another example, the ASO may have at least 95% (e.g., at least 95%, 96%, 97%, 98%, 99%, or more) sequence identity to the nucleic acid sequence of SEQ ID NO: 23. In a further example, the ASO may have the nucleic acid sequence of SEQ ID NO: 23.

An ASO sequence of the present disclosure may have at least 85% (e.g., at least 86%, 90%, 95%, 96%, 97%, 98%, 99%, or more) sequence identity to the nucleic acid sequence of SEQ ID NO: 24. For example, the ASO may have at least 90% (e.g., at least 90%, 95%, 96%, 97%, 98%, 99%, or more) sequence identity to the nucleic acid sequence of SEQ ID NO: 24. In another example, the ASO may have at least 95% (e.g., at least 95%, 96%, 97%, 98%, 99%, or more) sequence identity to the nucleic acid sequence of SEQ ID NO: 24. In a further example, the ASO may have the nucleic acid sequence of SEQ ID NO: 24.

An ASO sequence of the present disclosure may have at least 85% (e.g., at least 86%, 90%, 95%, 96%, 97%, 98%, 99%, or more) sequence identity to the nucleic acid sequence of SEQ ID NO: 25. For example, the ASO may have at least 90% (e.g., at least 90%, 95%, 96%, 97%, 98%, 99%, or more) sequence identity to the nucleic acid sequence of SEQ ID NO: 25. In another example, the ASO may have at least 95% (e.g., at least 95%, 96%, 97%, 98%, 99%, or more) sequence identity to the nucleic acid sequence of SEQ ID NO: 25. In a further example, the ASO may have the nucleic acid sequence of SEQ ID NO: 25.

An ASO sequence of the present disclosure may have at least 85% (e.g., at least 86%, 90%, 95%, 96%, 97%, 98%, 99%, or more) sequence identity to the nucleic acid sequence of SEQ ID NO: 26. For example, the ASO may have at least 90% (e.g., at least 90%, 95%, 96%, 97%, 98%, 99%, or more) sequence identity to the nucleic acid sequence of SEQ ID NO: 26. In another example, the ASO may have at least 95% (e.g., at least 95%, 96%, 97%, 98%, 99%, or more) sequence identity to the nucleic acid sequence of SEQ ID NO: 26. In a further example, the ASO may have the nucleic acid sequence of SEQ ID NO: 26.

An ASO sequence of the present disclosure may have at least 85% (e.g., at least 86%, 90%, 95%, 96%, 97%, 98%, 99%, or more) sequence identity to the nucleic acid sequence of SEQ ID NO: 27. For example, the ASO may have at least 90% (e.g., at least 90%, 95%, 96%, 97%, 98%, 99%, or more) sequence identity to the nucleic acid sequence of SEQ ID NO: 27. In another example, the ASO may have at least 95% (e.g., at least 95%, 96%, 97%, 98%, 99%, or more) sequence identity to the nucleic acid sequence of SEQ ID NO: 27. In a further example, the ASO may have the nucleic acid sequence of SEQ ID NO: 27.

An ASO sequence of the present disclosure may have at least 85% (e.g., at least 86%, 90%, 95%, 96%, 97%, 98%, 99%, or more) sequence identity to the nucleic acid sequence of SEQ ID NO: 28. For example, the ASO may have at least 90% (e.g., at least 90%, 95%, 96%, 97%, 98%, 99%, or more) sequence identity to the nucleic acid sequence of SEQ ID NO: 28. In another example, the ASO may have at least 95% (e.g., at least 95%, 96%, 97%, 98%, 99%, or more) sequence identity to the nucleic acid sequence of SEQ ID NO: 28. In a further example, the ASO may have the nucleic acid sequence of SEQ ID NO: 28.

An ASO sequence of the present disclosure may have at least 85% (e.g., at least 86%, 90%, 95%, 96%, 97%, 98%, 99%, or more) sequence identity to the nucleic acid sequence of SEQ ID NO: 29. For example, the ASO may have at least 90% (e.g., at least 90%, 95%, 96%, 97%, 98%, 99%, or more) sequence identity to the nucleic acid sequence of SEQ ID NO: 29. In another example, the ASO may have at least 95% (e.g., at least 95%, 96%, 97%, 98%, 99%, or more) sequence identity to the nucleic acid sequence of SEQ ID NO: 29. In a further example, the ASO may have the nucleic acid sequence of SEQ ID NO: 29.

An ASO sequence of the present disclosure may have at least 85% (e.g., at least 86%, 90%, 95%, 96%, 97%, 98%, 99%, or more) sequence identity to the nucleic acid sequence of SEQ ID NO: 30. For example, the ASO may have at least 90% (e.g., at least 90%, 95%, 96%, 97%, 98%, 99%, or more) sequence identity to the nucleic acid sequence of SEQ ID NO: 30. In another example, the ASO may have at least 95% (e.g., at least 95%, 96%, 97%, 98%, 99%, or more) sequence identity to the nucleic acid sequence of SEQ ID NO: 30. In a further example, the ASO may have the nucleic acid sequence of SEQ ID NO: 30.

An ASO sequence of the present disclosure may have at least 85% (e.g., at least 86%, 90%, 95%, 96%, 97%, 98%, 99%, or more) sequence identity to the nucleic acid sequence of SEQ ID NO: 31. For example, the ASO may have at least 90% (e.g., at least 90%, 95%, 96%, 97%, 98%, 99%, or more) sequence identity to the nucleic acid sequence of SEQ ID NO: 31. In another example, the ASO may have at least 95% (e.g., at least 95%, 96%, 97%, 98%, 99%, or more) sequence identity to the nucleic acid sequence of SEQ ID NO: 31. In a further example, the ASO may have the nucleic acid sequence of SEQ ID NO: 31.

An ASO sequence of the present disclosure may have at least 85% (e.g., at least 86%, 90%, 95%, 96%, 97%, 98%, 99%, or more) sequence identity to the nucleic acid sequence of SEQ ID NO: 32. For example, the ASO may have at least 90% (e.g., at least 90%, 95%, 96%, 97%, 98%, 99%, or more) sequence identity to the nucleic acid sequence of SEQ ID NO: 32. In another example, the ASO may have at least 95% (e.g., at least 95%, 96%, 97%, 98%, 99%, or more) sequence identity to the nucleic acid sequence of SEQ ID NO: 32. In a further example, the ASO may have the nucleic acid sequence of SEQ ID NO: 32.

An ASO sequence of the present disclosure may have at least 85% (e.g., at least 86%, 90%, 95%, 96%, 97%, 98%, 99%, or more) sequence identity to the nucleic acid sequence of SEQ ID NO: 33. For example, the ASO may have at least 90% (e.g., at least 90%, 95%, 96%, 97%, 98%, 99%, or more) sequence identity to the nucleic acid sequence of SEQ ID NO: 33. In another example, the ASO may have at least 95% (e.g., at least 95%, 96%, 97%, 98%, 99%, or more) sequence identity to the nucleic acid sequence of SEQ ID NO: 33. In a further example, the ASO may have the nucleic acid sequence of SEQ ID NO: 33.

An ASO sequence of the present disclosure may have at least 85% (e.g., at least 86%, 90%, 95%, 96%, 97%, 98%, 99%, or more) sequence identity to the nucleic acid sequence of SEQ ID NO: 34. For example, the ASO may have at least 90% (e.g., at least 90%, 95%, 96%, 97%, 98%, 99%, or more) sequence identity to the nucleic acid sequence of SEQ ID NO: 34. In another example, the ASO may have at least 95% (e.g., at least 95%, 96%, 97%, 98%, 99%, or more) sequence identity to the nucleic acid sequence of SEQ ID NO: 34. In a further example, the ASO may have the nucleic acid sequence of SEQ ID NO: 34.

An ASO sequence of the present disclosure may have at least 85% (e.g., at least 86%, 90%, 95%, 96%, 97%, 98%, 99%, or more) sequence identity to the nucleic acid sequence of SEQ ID NO: 35. For example, the ASO may have at least 90% (e.g., at least 90%, 95%, 96%, 97%, 98%, 99%, or more) sequence identity to the nucleic acid sequence of SEQ ID NO: 35. In another example, the ASO may have at least 95% (e.g., at least 95%, 96%, 97%, 98%, 99%, or more) sequence identity to the nucleic acid sequence of SEQ ID NO: 35. In a further example, the ASO may have the nucleic acid sequence of SEQ ID NO: 35.

An ASO sequence of the present disclosure may have at least 85% (e.g., at least 86%, 90%, 95%, 96%, 97%, 98%, 99%, or more) sequence identity to the nucleic acid sequence of SEQ ID NO: 36. For example, the ASO may have at least 90% (e.g., at least 90%, 95%, 96%, 97%, 98%, 99%, or more) sequence identity to the nucleic acid sequence of SEQ ID NO: 36. In another example, the ASO may have at least 95% (e.g., at least 95%, 96%, 97%, 98%, 99%, or more) sequence identity to the nucleic acid sequence of SEQ ID NO: 36. In a further example, the ASO may have the nucleic acid sequence of SEQ ID NO: 36.

An ASO sequence of the present disclosure may have at least 85% (e.g., at least 86%, 90%, 95%, 96%, 97%, 98%, 99%, or more) sequence identity to the nucleic acid sequence of SEQ ID NO: 37. For example, the ASO may have at least 90% (e.g., at least 90%, 95%, 96%, 97%, 98%, 99%, or more) sequence identity to the nucleic acid sequence of SEQ ID NO: 37. In another example, the ASO may have at least 95% (e.g., at least 95%, 96%, 97%, 98%, 99%, or more) sequence identity to the nucleic acid sequence of SEQ ID NO: 37. In a further example, the ASO may have the nucleic acid sequence of SEQ ID NO: 37.

An ASO sequence of the present disclosure may have at least 85% (e.g., at least 86%, 90%, 95%, 96%, 97%, 98%, 99%, or more) sequence identity to the nucleic acid sequence of SEQ ID NO: 38. For example, the ASO may have at least 90% (e.g., at least 90%, 95%, 96%, 97%, 98%, 99%, or more) sequence identity to the nucleic acid sequence of SEQ ID NO: 38. In another example, the ASO may have at least 95% (e.g., at least 95%, 96%, 97%, 98%, 99%, or more) sequence identity to the nucleic acid sequence of SEQ ID NO: 38. In a further example, the ASO may have the nucleic acid sequence of SEQ ID NO: 38.

An ASO sequence of the present disclosure may have at least 85% (e.g., at least 86%, 90%, 95%, 96%, 97%, 98%, 99%, or more) sequence identity to the nucleic acid sequence of SEQ ID NO: 39. For example, the ASO may have at least 90% (e.g., at least 90%, 95%, 96%, 97%, 98%, 99%, or more) sequence identity to the nucleic acid sequence of SEQ ID NO: 39. In another example, the ASO may have at least 95% (e.g., at least 95%, 96%, 97%, 98%, 99%, or more) sequence identity to the nucleic acid sequence of SEQ ID NO: 39. In a further example, the ASO may have the nucleic acid sequence of SEQ ID NO: 39.

An ASO sequence of the present disclosure may have at least 85% (e.g., at least 86%, 90%, 95%, 96%, 97%, 98%, 99%, or more) sequence identity to the nucleic acid sequence of SEQ ID NO: 40. For example, the ASO may have at least 90% (e.g., at least 90%, 95%, 96%, 97%, 98%, 99%, or more) sequence identity to the nucleic acid sequence of SEQ ID NO: 40. In another example, the ASO may have at least 95% (e.g., at least 95%, 96%, 97%, 98%, 99%, or more) sequence identity to the nucleic acid sequence of SEQ ID NO: 40. In a further example, the ASO may have the nucleic acid sequence of SEQ ID NO: 40.

An ASO sequence of the present disclosure may have at least 85% (e.g., at least 86%, 90%, 95%, 96%, 97%, 98%, 99%, or more) sequence identity to the nucleic acid sequence of SEQ ID NO: 41. For example, the ASO may have at least 90% (e.g., at least 90%, 95%, 96%, 97%, 98%, 99%, or more) sequence identity to the nucleic acid sequence of SEQ ID NO: 41. In another example, the ASO may have at least 95% (e.g., at least 95%, 96%, 97%, 98%, 99%, or more) sequence identity to the nucleic acid sequence of SEQ ID NO: 41. In a further example, the ASO may have the nucleic acid sequence of SEQ ID NO: 41.

An ASO sequence of the present disclosure may have at least 85% (e.g., at least 86%, 90%, 95%, 96%, 97%, 98%, 99%, or more) sequence identity to the nucleic acid sequence of SEQ ID NO: 42. For example, the ASO may have at least 90% (e.g., at least 90%, 95%, 96%, 97%, 98%, 99%, or more) sequence identity to the nucleic acid sequence of SEQ ID NO: 42. In another example, the ASO may have at least 95% (e.g., at least 95%, 96%, 97%, 98%, 99%, or more) sequence identity to the nucleic acid sequence of SEQ ID NO: 42. In a further example, the ASO may have the nucleic acid sequence of SEQ ID NO: 42.

An ASO sequence of the present disclosure may have at least 85% (e.g., at least 86%, 90%, 95%, 96%, 97%, 98%, 99%, or more) sequence identity to the nucleic acid sequence of SEQ ID NO: 43. For example, the ASO may have at least 90% (e.g., at least 90%, 95%, 96%, 97%, 98%, 99%, or more) sequence identity to the nucleic acid sequence of SEQ ID NO: 43. In another example, the ASO may have at least 95% (e.g., at least 95%, 96%, 97%, 98%, 99%, or more) sequence identity to the nucleic acid sequence of SEQ ID NO: 43. In a further example, the ASO may have the nucleic acid sequence of SEQ ID NO: 43.

An ASO sequence of the present disclosure may have at least 85% (e.g., at least 86%, 90%, 95%, 96%, 97%, 98%, 99%, or more) sequence identity to the nucleic acid sequence of SEQ ID NO: 44. For example, the ASO may have at least 90% (e.g., at least 90%, 95%, 96%, 97%, 98%, 99%, or more) sequence identity to the nucleic acid sequence of SEQ ID NO: 44. In another example, the ASO may have at least 95% (e.g., at least 95%, 96%, 97%, 98%, 99%, or more) sequence identity to the nucleic acid sequence of SEQ ID NO: 44. In a further example, the ASO may have the nucleic acid sequence of SEQ ID NO: 44.

An ASO sequence of the present disclosure may have at least 85% (e.g., at least 86%, 90%, 95%, 96%, 97%, 98%, 99%, or more) sequence identity to the nucleic acid sequence of SEQ ID NO: 45. For example, the ASO may have at least 90% (e.g., at least 90%, 95%, 96%, 97%, 98%, 99%, or more) sequence identity to the nucleic acid sequence of SEQ ID NO: 45. In another example, the ASO may have at least 95% (e.g., at least 95%, 96%, 97%, 98%, 99%, or more) sequence identity to the nucleic acid sequence of SEQ ID NO: 45. In a further example, the ASO may have the nucleic acid sequence of SEQ ID NO: 45.

An ASO sequence of the present disclosure may have at least 85% (e.g., at least 86%, 90%, 95%, 96%, 97%, 98%, 99%, or more) sequence identity to the nucleic acid sequence of SEQ ID NO: 46. For example, the ASO may have at least 90% (e.g., at least 90%, 95%, 96%, 97%, 98%, 99%, or more) sequence identity to the nucleic acid sequence of SEQ ID NO: 46. In another example, the ASO may have at least 95% (e.g., at least 95%, 96%, 97%, 98%, 99%, or more) sequence identity to the nucleic acid sequence of SEQ ID NO: 46. In a further example, the ASO may have the nucleic acid sequence of SEQ ID NO: 46.

An ASO sequence of the present disclosure may have at least 85% (e.g., at least 86%, 90%, 95%, 96%, 97%, 98%, 99%, or more) sequence identity to the nucleic acid sequence of SEQ ID NO: 47. For example, the ASO may have at least 90% (e.g., at least 90%, 95%, 96%, 97%, 98%, 99%, or more) sequence identity to the nucleic acid sequence of SEQ ID NO: 47. In another example, the ASO may have at least 95% (e.g., at least 95%, 96%, 97%, 98%, 99%, or more) sequence identity to the nucleic acid sequence of SEQ ID NO: 47. In a further example, the ASO may have the nucleic acid sequence of SEQ ID NO: 47.

An ASO sequence of the present disclosure may have at least 85% (e.g., at least 86%, 90%, 95%, 96%, 97%, 98%, 99%, or more) sequence identity to the nucleic acid sequence of SEQ ID NO: 48. For example, the ASO may have at least 90% (e.g., at least 90%, 95%, 96%, 97%, 98%, 99%, or more) sequence identity to the nucleic acid sequence of SEQ ID NO: 48. In another example, the ASO may have at least 95% (e.g., at least 95%, 96%, 97%, 98%, 99%, or more) sequence identity to the nucleic acid sequence of SEQ ID NO: 48. In a further example, the ASO may have the nucleic acid sequence of SEQ ID NO: 48.

An ASO sequence of the present disclosure may have at least 85% (e.g., at least 86%, 90%, 95%, 96%, 97%, 98%, 99%, or more) sequence identity to the nucleic acid sequence of SEQ ID NO: 49. For example, the ASO may have at least 90% (e.g., at least 90%, 95%, 96%, 97%, 98%, 99%, or more) sequence identity to the nucleic acid sequence of SEQ ID NO: 49. In another example, the ASO may have at least 95% (e.g., at least 95%, 96%, 97%, 98%, 99%, or more) sequence identity to the nucleic acid sequence of SEQ ID NO: 49. In a further example, the ASO may have the nucleic acid sequence of SEQ ID NO: 49.

An ASO sequence of the present disclosure may have at least 85% (e.g., at least 86%, 90%, 95%, 96%, 97%, 98%, 99%, or more) sequence identity to the nucleic acid sequence of SEQ ID NO: 50. For example, the ASO may have at least 90% (e.g., at least 90%, 95%, 96%, 97%, 98%, 99%, or more) sequence identity to the nucleic acid sequence of SEQ ID NO: 50. In another example, the ASO may have at least 95% (e.g., at least 95%, 96%, 97%, 98%, 99%, or more) sequence identity to the nucleic acid sequence of SEQ ID NO: 50. In a further example, the ASO may have the nucleic acid sequence of SEQ ID NO: 50.

An ASO sequence of the present disclosure may have at least 85% (e.g., at least 86%, 90%, 95%, 96%, 97%, 98%, 99%, or more) sequence identity to the nucleic acid sequence of SEQ ID NO: 51. For example, the ASO may have at least 90% (e.g., at least 90%, 95%, 96%, 97%, 98%, 99%, or more) sequence identity to the nucleic acid sequence of SEQ ID NO: 51. In another example, the ASO may have at least 95% (e.g., at least 95%, 96%, 97%, 98%, 99%, or more) sequence identity to the nucleic acid sequence of SEQ ID NO: 51. In a further example, the ASO may have the nucleic acid sequence of SEQ ID NO: 51.

An ASO sequence of the present disclosure may have at least 85% (e.g., at least 86%, 90%, 95%, 96%, 97%, 98%, 99%, or more) sequence identity to the nucleic acid sequence of SEQ ID NO: 52. For example, the ASO may have at least 90% (e.g., at least 90%, 95%, 96%, 97%, 98%, 99%, or more) sequence identity to the nucleic acid sequence of SEQ ID NO: 52. In another example, the ASO may have at least 95% (e.g., at least 95%, 96%, 97%, 98%, 99%, or more) sequence identity to the nucleic acid sequence of SEQ ID NO: 52. In a further example, the ASO may have the nucleic acid sequence of SEQ ID NO: 52.

An ASO sequence of the present disclosure may have at least 85% (e.g., at least 86%, 90%, 95%, 96%, 97%, 98%, 99%, or more) sequence identity to the nucleic acid sequence of SEQ ID NO: 53. For example, the ASO may have at least 90% (e.g., at least 90%, 95%, 96%, 97%, 98%, 99%, or more) sequence identity to the nucleic acid sequence of SEQ ID NO: 53. In another example, the ASO may have at least 95% (e.g., at least 95%, 96%, 97%, 98%, 99%, or more) sequence identity to the nucleic acid sequence of SEQ ID NO: 53. In a further example, the ASO may have the nucleic acid sequence of SEQ ID NO: 53.

An ASO sequence of the present disclosure may have at least 85% (e.g., at least 86%, 90%, 95%, 96%, 97%, 98%, 99%, or more) sequence identity to the nucleic acid sequence of SEQ ID NO: 54. For example, the ASO may have at least 90% (e.g., at least 90%, 95%, 96%, 97%, 98%, 99%, or more) sequence identity to the nucleic acid sequence of SEQ ID NO: 54. In another example, the ASO may have at least 95% (e.g., at least 95%, 96%, 97%, 98%, 99%, or more) sequence identity to the nucleic acid sequence of SEQ ID NO: 54. In a further example, the ASO may have the nucleic acid sequence of SEQ ID NO: 54.

An ASO sequence of the present disclosure may have at least 85% (e.g., at least 86%, 90%, 95%, 96%, 97%, 98%, 99%, or more) sequence identity to the nucleic acid sequence of SEQ ID NO: 55. For example, the ASO may have at least 90% (e.g., at least 90%, 95%, 96%, 97%, 98%, 99%, or more) sequence identity to the nucleic acid sequence of SEQ ID NO: 55. In another example, the ASO may have at least 95% (e.g., at least 95%, 96%, 97%, 98%, 99%, or more) sequence identity to the nucleic acid sequence of SEQ ID NO: 55. In a further example, the ASO may have the nucleic acid sequence of SEQ ID NO: 55.

An ASO sequence of the present disclosure may have at least 85% (e.g., at least 86%, 90%, 95%, 96%, 97%, 98%, 99%, or more) sequence identity to the nucleic acid sequence of SEQ ID NO: 56. For example, the ASO may have at least 90% (e.g., at least 90%, 95%, 96%, 97%, 98%, 99%, or more) sequence identity to the nucleic acid sequence of SEQ ID NO: 56. In another example, the ASO may have at least 95% (e.g., at least 95%, 96%, 97%, 98%, 99%, or more) sequence identity to the nucleic acid sequence of SEQ ID NO: 56. In a further example, the ASO may have the nucleic acid sequence of SEQ ID NO: 56.

An ASO sequence of the present disclosure may have at least 85% (e.g., at least 86%, 90%, 95%, 25 96%, 97%, 98%, 99%, or more) sequence identity to the nucleic acid sequence of SEQ ID NO: 57. For example, the ASO may have at least 90% (e.g., at least 90%, 95%, 96%, 97%, 98%, 99%, or more) sequence identity to the nucleic acid sequence of SEQ ID NO: 57. In another example, the ASO may have at least 95% (e.g., at least 95%, 96%, 97%, 98%, 99%, or more) sequence identity to the nucleic acid sequence of SEQ ID NO: 57. In a further example, the ASO may have the nucleic acid sequence of SEQ ID NO: 57.

An ASO sequence of the present disclosure may have at least 85% (e.g., at least 86%, 90%, 95%, 96%, 97%, 98%, 99%, or more) sequence identity to the nucleic acid sequence of SEQ ID NO: 58. For example, the ASO may have at least 90% (e.g., at least 90%, 95%, 96%, 97%, 98%, 99%, or more) sequence identity to the nucleic acid sequence of SEQ ID NO: 58. In another example, the ASO may have at least 95% (e.g., at least 95%, 96%, 97%, 98%, 99%, or more) sequence identity to the nucleic acid sequence of SEQ ID NO: 58. In a further example, the ASO may have the nucleic acid sequence of SEQ ID NO: 58.

An ASO sequence of the present disclosure may have at least 85% (e.g., at least 86%, 90%, 95%, 96%, 97%, 98%, 99%, or more) sequence identity to the nucleic acid sequence of SEQ ID NO: 59. For example, the ASO may have at least 90% (e.g., at least 90%, 95%, 96%, 97%, 98%, 99%, or more) sequence identity to the nucleic acid sequence of SEQ ID NO: 59. In another example, the ASO may have at least 95% (e.g., at least 95%, 96%, 97%, 98%, 99%, or more) sequence identity to the nucleic acid sequence of SEQ ID NO: 59. In a further example, the ASO may have the nucleic acid sequence of SEQ ID NO: 59.

An ASO sequence of the present disclosure may have at least 85% (e.g., at least 86%, 90%, 95%, 96%, 97%, 98%, 99%, or more) sequence identity to the nucleic acid sequence of SEQ ID NO: 60. For example, the ASO may have at least 90% (e.g., at least 90%, 95%, 96%, 97%, 98%, 99%, or more) sequence identity to the nucleic acid sequence of SEQ ID NO: 60. In another example, the ASO may have at least 95% (e.g., at least 95%, 96%, 97%, 98%, 99%, or more) sequence identity to the nucleic acid sequence of SEQ ID NO: 60. In a further example, the ASO may have the nucleic acid sequence of SEQ ID NO: 60.

An ASO sequence of the present disclosure may have at least 85% (e.g., at least 86%, 90%, 95%, 96%, 97%, 98%, 99%, or more) sequence identity to the nucleic acid sequence of SEQ ID NO: 61. For example, the ASO may have at least 90% (e.g., at least 90%, 95%, 96%, 97%, 98%, 99%, or more) sequence identity to the nucleic acid sequence of SEQ ID NO: 61. In another example, the ASO may have at least 95% (e.g., at least 95%, 96%, 97%, 98%, 99%, or more) sequence identity to the nucleic acid sequence of SEQ ID NO: 61. In a further example, the ASO may have the nucleic acid sequence of SEQ ID NO: 61.

An ASO sequence of the present disclosure may have at least 85% (e.g., at least 86%, 90%, 95%, 96%, 97%, 98%, 99%, or more) sequence identity to the nucleic acid sequence of SEQ ID NO: 62. For example, the ASO may have at least 90% (e.g., at least 90%, 95%, 96%, 97%, 98%, 99%, or more) sequence identity to the nucleic acid sequence of SEQ ID NO: 62. In another example, the ASO may have at least 95% (e.g., at least 95%, 96%, 97%, 98%, 99%, or more) sequence identity to the nucleic acid sequence of SEQ ID NO: 62. In a further example, the ASO may have the nucleic acid sequence of SEQ ID NO: 62.

An ASO sequence of the present disclosure may have at least 85% (e.g., at least 86%, 90%, 95%, 96%, 97%, 98%, 99%, or more) sequence identity to the nucleic acid sequence of SEQ ID NO: 63. For example, the ASO may have at least 90% (e.g., at least 90%, 95%, 96%, 97%, 98%, 99%, or more) sequence identity to the nucleic acid sequence of SEQ ID NO: 63. In another example, the ASO may have at least 95% (e.g., at least 95%, 96%, 97%, 98%, 99%, or more) sequence identity to the nucleic acid sequence of SEQ ID NO: 63. In a further example, the ASO may have the nucleic acid sequence of SEQ ID NO: 63.

An ASO sequence of the present disclosure may have at least 85% (e.g., at least 86%, 90%, 95%, 96%, 97%, 98%, 99%, or more) sequence identity to the nucleic acid sequence of SEQ ID NO: 64. For example, the ASO may have at least 90% (e.g., at least 90%, 95%, 96%, 97%, 98%, 99%, or more) sequence identity to the nucleic acid sequence of SEQ ID NO: 64. In another example, the ASO may have at least 95% (e.g., at least 95%, 96%, 97%, 98%, 99%, or more) sequence identity to the nucleic acid sequence of SEQ ID NO: 64. In a further example, the ASO may have the nucleic acid sequence of SEQ ID NO: 64.

An ASO sequence of the present disclosure may have at least 85% (e.g., at least 86%, 90%, 95%, 96%, 97%, 98%, 99%, or more) sequence identity to the nucleic acid sequence of SEQ ID NO: 65. For example, the ASO may have at least 90% (e.g., at least 90%, 95%, 96%, 97%, 98%, 99%, or more) sequence identity to the nucleic acid sequence of SEQ ID NO: 65. In another example, the ASO may have at least 95% (e.g., at least 95%, 96%, 97%, 98%, 99%, or more) sequence identity to the nucleic acid sequence of SEQ ID NO: 65. In a further example, the ASO may have the nucleic acid sequence of SEQ ID NO: 65.

An ASO sequence of the present disclosure may have at least 85% (e.g., at least 86%, 90%, 95%, 96%, 97%, 98%, 99%, or more) sequence identity to the nucleic acid sequence of SEQ ID NO: 66. For example, the ASO may have at least 90% (e.g., at least 90%, 95%, 96%, 97%, 98%, 99%, or more) sequence identity to the nucleic acid sequence of SEQ ID NO: 66. In another example, the ASO may have at least 95% (e.g., at least 95%, 96%, 97%, 98%, 99%, or more) sequence identity to the nucleic acid sequence of SEQ ID NO: 66. In a further example, the ASO may have the nucleic acid sequence of SEQ ID NO: 66.

An ASO sequence of the present disclosure may have at least 85% (e.g., at least 86%, 90%, 95%, 96%, 97%, 98%, 99%, or more) sequence identity to the nucleic acid sequence of SEQ ID NO: 67. For example, the ASO may have at least 90% (e.g., at least 90%, 95%, 96%, 97%, 98%, 99%, or more) sequence identity to the nucleic acid sequence of SEQ ID NO: 67. In another example, the ASO may have at least 95% (e.g., at least 95%, 96%, 97%, 98%, 99%, or more) sequence identity to the nucleic acid sequence of SEQ ID NO: 67. In a further example, the ASO may have the nucleic acid sequence of SEQ ID NO: 67.

An ASO sequence of the present disclosure may have at least 85% (e.g., at least 86%, 90%, 95%, 96%, 97%, 98%, 99%, or more) sequence identity to the nucleic acid sequence of SEQ ID NO: 68. For example, the ASO may have at least 90% (e.g., at least 90%, 95%, 96%, 97%, 98%, 99%, or more) sequence identity to the nucleic acid sequence of SEQ ID NO: 68. In another example, the ASO may have at least 95% (e.g., at least 95%, 96%, 97%, 98%, 99%, or more) sequence identity to the nucleic acid sequence of SEQ ID NO: 68. In a further example, the ASO may have the nucleic acid sequence of SEQ ID NO: 68.

An ASO sequence of the present disclosure may have at least 85% (e.g., at least 86%, 90%, 95%, 96%, 97%, 98%, 99%, or more) sequence identity to the nucleic acid sequence of SEQ ID NO: 69. For example, the ASO may have at least 90% (e.g., at least 90%, 95%, 96%, 97%, 98%, 99%, or more) sequence identity to the nucleic acid sequence of SEQ ID NO: 69. In another example, the ASO may have at least 95% (e.g., at least 95%, 96%, 97%, 98%, 99%, or more) sequence identity to the nucleic acid sequence of SEQ ID NO: 69. In a further example, the ASO may have the nucleic acid sequence of SEQ ID NO: 69.

An ASO sequence of the present disclosure may have at least 85% (e.g., at least 86%, 90%, 95%, 96%, 97%, 98%, 99%, or more) sequence identity to the nucleic acid sequence of SEQ ID NO: 70. For example, the ASO may have at least 90% (e.g., at least 90%, 95%, 96%, 97%, 98%, 99%, or more) sequence identity to the nucleic acid sequence of SEQ ID NO: 70. In another example, the ASO may have at least 95% (e.g., at least 95%, 96%, 97%, 98%, 99%, or more) sequence identity to the nucleic acid sequence of SEQ ID NO: 70. In a further example, the ASO may have the nucleic acid sequence of SEQ ID NO: 70.

An ASO sequence of the present disclosure may have at least 85% (e.g., at least 86%, 90%, 95%, 96%, 97%, 98%, 99%, or more) sequence identity to the nucleic acid sequence of SEQ ID NO: 71. For example, the ASO may have at least 90% (e.g., at least 90%, 95%, 96%, 97%, 98%, 99%, or more) sequence identity to the nucleic acid sequence of SEQ ID NO: 71. In another example, the ASO may have at least 95% (e.g., at least 95%, 96%, 97%, 98%, 99%, or more) sequence identity to the nucleic acid sequence of SEQ ID NO: 71. In a further example, the ASO may have the nucleic acid sequence of SEQ ID NO: 71.

An ASO sequence of the present disclosure may have at least 85% (e.g., at least 86%, 90%, 95%, 96%, 97%, 98%, 99%, or more) sequence identity to the nucleic acid sequence of SEQ ID NO: 72. For example, the ASO may have at least 90% (e.g., at least 90%, 95%, 96%, 97%, 98%, 99%, or more) sequence identity to the nucleic acid sequence of SEQ ID NO: 72. In another example, the ASO may have at least 95% (e.g., at least 95%, 96%, 97%, 98%, 99%, or more) sequence identity to the nucleic acid sequence of SEQ ID NO: 72. In a further example, the ASO may have the nucleic acid sequence of SEQ ID NO: 72.

An ASO sequence of the present disclosure may have at least 85% (e.g., at least 86%, 90%, 95%, 96%, 97%, 98%, 99%, or more) sequence identity to the nucleic acid sequence of SEQ ID NO: 73. For example, the ASO may have at least 90% (e.g., at least 90%, 95%, 96%, 97%, 98%, 99%, or more) sequence identity to the nucleic acid sequence of SEQ ID NO: 73. In another example, the ASO may have at least 95% (e.g., at least 95%, 96%, 97%, 98%, 99%, or more) sequence identity to the nucleic acid sequence of SEQ ID NO: 73. In a further example, the ASO may have the nucleic acid sequence of SEQ ID NO: 73.

An ASO sequence of the present disclosure may have at least 85% (e.g., at least 86%, 90%, 95%, 96%, 97%, 98%, 99%, or more) sequence identity to the nucleic acid sequence of SEQ ID NO: 74. For example, the ASO may have at least 90% (e.g., at least 90%, 95%, 96%, 97%, 98%, 99%, or more) sequence identity to the nucleic acid sequence of SEQ ID NO: 74. In another example, the ASO may have at least 95% (e.g., at least 95%, 96%, 97%, 98%, 99%, or more) sequence identity to the nucleic acid sequence of SEQ ID NO: 74. In a further example, the ASO may have the nucleic acid sequence of SEQ ID NO: 74.

An ASO sequence of the present disclosure may have at least 85% (e.g., at least 86%, 90%, 95%, 96%, 97%, 98%, 99%, or more) sequence identity to the nucleic acid sequence of SEQ ID NO: 75. For example, the ASO may have at least 90% (e.g., at least 90%, 95%, 96%, 97%, 98%, 99%, or more) sequence identity to the nucleic acid sequence of SEQ ID NO: 75. In another example, the ASO may have at least 95% (e.g., at least 95%, 96%, 97%, 98%, 99%, or more) sequence identity to the nucleic acid sequence of SEQ ID NO: 75. In a further example, the ASO may have the nucleic acid sequence of SEQ ID NO: 75.

An ASO sequence of the present disclosure may have at least 85% (e.g., at least 86%, 90%, 95%, 96%, 97%, 98%, 99%, or more) sequence identity to the nucleic acid sequence of SEQ ID NO: 76. For example, the ASO may have at least 90% (e.g., at least 90%, 95%, 96%, 97%, 98%, 99%, or more) sequence identity to the nucleic acid sequence of SEQ ID NO: 76. In another example, the ASO may have at least 95% (e.g., at least 95%, 96%, 97%, 98%, 99%, or more) sequence identity to the nucleic acid sequence of SEQ ID NO: 76. In a further example, the ASO may have the nucleic acid sequence of SEQ ID NO: 76.

An ASO sequence of the present disclosure may have at least 85% (e.g., at least 86%, 90%, 95%, 96%, 97%, 98%, 99%, or more) sequence identity to the nucleic acid sequence of SEQ ID NO: 77. For example, the ASO may have at least 90% (e.g., at least 90%, 95%, 96%, 97%, 98%, 99%, or more) sequence identity to the nucleic acid sequence of SEQ ID NO: 77. In another example, the ASO may have at least 95% (e.g., at least 95%, 96%, 97%, 98%, 99%, or more) sequence identity to the nucleic acid sequence of SEQ ID NO: 77. In a further example, the ASO may have the nucleic acid sequence of SEQ ID NO: 77.

An ASO sequence of the present disclosure may have at least 85% (e.g., at least 86%, 90%, 95%, 96%, 97%, 98%, 99%, or more) sequence identity to the nucleic acid sequence of SEQ ID NO: 78. For example, the ASO may have at least 90% (e.g., at least 90%, 95%, 96%, 97%, 98%, 99%, or more) sequence identity to the nucleic acid sequence of SEQ ID NO: 78. In another example, the ASO may have at least 95% (e.g., at least 95%, 96%, 97%, 98%, 99%, or more) sequence identity to the nucleic acid sequence of SEQ ID NO: 78. In a further example, the ASO may have the nucleic acid sequence of SEQ ID NO: 78.

An ASO sequence of the present disclosure may have at least 85% (e.g., at least 86%, 90%, 95%, 96%, 97%, 98%, 99%, or more) sequence identity to the nucleic acid sequence of SEQ ID NO: 79. For example, the ASO may have at least 90% (e.g., at least 90%, 95%, 96%, 97%, 98%, 99%, or more) sequence identity to the nucleic acid sequence of SEQ ID NO: 79. In another example, the ASO may have at least 95% (e.g., at least 95%, 96%, 97%, 98%, 99%, or more) sequence identity to the nucleic acid sequence of SEQ ID NO: 79. In a further example, the ASO may have the nucleic acid sequence of SEQ ID NO: 79.

An ASO sequence of the present disclosure may have at least 85% (e.g., at least 86%, 90%, 95%, 96%, 97%, 98%, 99%, or more) sequence identity to the nucleic acid sequence of SEQ ID NO: 80. For example, the ASO may have at least 90% (e.g., at least 90%, 95%, 96%, 97%, 98%, 99%, or more) sequence identity to the nucleic acid sequence of SEQ ID NO: 80. In another example, the ASO may have at least 95% (e.g., at least 95%, 96%, 97%, 98%, 99%, or more) sequence identity to the nucleic acid sequence of SEQ ID NO: 80. In a further example, the ASO may have the nucleic acid sequence of SEQ ID NO: 80.

An ASO sequence of the present disclosure may have at least 85% (e.g., at least 86%, 90%, 95%, 96%, 97%, 98%, 99%, or more) sequence identity to the nucleic acid sequence of SEQ ID NO: 81. For example, the ASO may have at least 90% (e.g., at least 90%, 95%, 96%, 97%, 98%, 99%, or more) sequence identity to the nucleic acid sequence of SEQ ID NO: 81. In another example, the ASO may have at least 95% (e.g., at least 95%, 96%, 97%, 98%, 99%, or more) sequence identity to the nucleic acid sequence of SEQ ID NO: 81. In a further example, the ASO may have the nucleic acid sequence of SEQ ID NO: 81.

An ASO sequence of the present disclosure may have at least 85% (e.g., at least 86%, 90%, 95%, 96%, 97%, 98%, 99%, or more) sequence identity to the nucleic acid sequence of SEQ ID NO: 82. For example, the ASO may have at least 90% (e.g., at least 90%, 95%, 96%, 97%, 98%, 99%, or more) sequence identity to the nucleic acid sequence of SEQ ID NO: 82. In another example, the ASO may have at least 95% (e.g., at least 95%, 96%, 97%, 98%, 99%, or more) sequence identity to the nucleic acid sequence of SEQ ID NO: 82. In a further example, the ASO may have the nucleic acid sequence of SEQ ID NO: 82.

An ASO sequence of the present disclosure may have at least 85% (e.g., at least 86%, 90%, 95%, 96%, 97%, 98%, 99%, or more) sequence identity to the nucleic acid sequence of SEQ ID NO: 83. For example, the ASO may have at least 90% (e.g., at least 90%, 95%, 96%, 97%, 98%, 99%, or more) sequence identity to the nucleic acid sequence of SEQ ID NO: 83. In another example, the ASO may have at least 95% (e.g., at least 95%, 96%, 97%, 98%, 99%, or more) sequence identity to the nucleic acid sequence of SEQ ID NO: 83. In a further example, the ASO may have the nucleic acid sequence of SEQ ID NO: 83.

An ASO sequence of the present disclosure may have at least 85% (e.g., at least 86%, 90%, 95%, 96%, 97%, 98%, 99%, or more) sequence identity to the nucleic acid sequence of SEQ ID NO: 84. For example, the ASO may have at least 90% (e.g., at least 90%, 95%, 96%, 97%, 98%, 99%, or more) sequence identity to the nucleic acid sequence of SEQ ID NO: 84. In another example, the ASO may have at least 95% (e.g., at least 95%, 96%, 97%, 98%, 99%, or more) sequence identity to the nucleic acid sequence of SEQ ID NO: 84. In a further example, the ASO may have the nucleic acid sequence of SEQ ID NO: 84.

An ASO sequence of the present disclosure may have at least 85% (e.g., at least 86%, 90%, 95%, 96%, 97%, 98%, 99%, or more) sequence identity to the nucleic acid sequence of SEQ ID NO: 85. For example, the ASO may have at least 90% (e.g., at least 90%, 95%, 96%, 97%, 98%, 99%, or more) sequence identity to the nucleic acid sequence of SEQ ID NO: 85. In another example, the ASO may have at least 95% (e.g., at least 95%, 96%, 97%, 98%, 99%, or more) sequence identity to the nucleic acid sequence of SEQ ID NO: 85. In a further example, the ASO may have the nucleic acid sequence of SEQ ID NO: 85.

An ASO sequence of the present disclosure may have at least 85% (e.g., at least 86%, 90%, 95%, 96%, 97%, 98%, 99%, or more) sequence identity to the nucleic acid sequence of SEQ ID NO: 86. For example, the ASO may have at least 90% (e.g., at least 90%, 95%, 96%, 97%, 98%, 99%, or more) sequence identity to the nucleic acid sequence of SEQ ID NO: 86. In another example, the ASO may have at least 95% (e.g., at least 95%, 96%, 97%, 98%, 99%, or more) sequence identity to the nucleic acid sequence of SEQ ID NO: 86. In a further example, the ASO may have the nucleic acid sequence of SEQ ID NO: 86.

An ASO sequence of the present disclosure may have at least 85% (e.g., at least 86%, 90%, 95%, 96%, 97%, 98%, 99%, or more) sequence identity to the nucleic acid sequence of SEQ ID NO: 87. For example, the ASO may have at least 90% (e.g., at least 90%, 95%, 96%, 97%, 98%, 99%, or more) sequence identity to the nucleic acid sequence of SEQ ID NO: 87. In another example, the ASO may have at least 95% (e.g., at least 95%, 96%, 97%, 98%, 99%, or more) sequence identity to the nucleic acid sequence of SEQ ID NO: 87. In a further example, the ASO may have the nucleic acid sequence of SEQ ID NO: 87.

An ASO sequence of the present disclosure may have at least 85% (e.g., at least 86%, 90%, 95%, 96%, 97%, 98%, 99%, or more) sequence identity to the nucleic acid sequence of SEQ ID NO: 88. For example, the ASO may have at least 90% (e.g., at least 90%, 95%, 96%, 97%, 98%, 99%, or more) sequence identity to the nucleic acid sequence of SEQ ID NO: 88. In another example, the ASO may have at least 95% (e.g., at least 95%, 96%, 97%, 98%, 99%, or more) sequence identity to the nucleic acid sequence of SEQ ID NO: 88. In a further example, the ASO may have the nucleic acid sequence of SEQ ID NO: 88.

An ASO sequence of the present disclosure may have at least 85% (e.g., at least 86%, 90%, 95%, 96%, 97%, 98%, 99%, or more) sequence identity to the nucleic acid sequence of SEQ ID NO: 89. For example, the ASO may have at least 90% (e.g., at least 90%, 95%, 96%, 97%, 98%, 99%, or more) sequence identity to the nucleic acid sequence of SEQ ID NO: 89. In another example, the ASO may have at least 95% (e.g., at least 95%, 96%, 97%, 98%, 99%, or more) sequence identity to the nucleic acid sequence of SEQ ID NO: 89. In a further example, the ASO may have the nucleic acid sequence of SEQ ID NO: 89.

An ASO sequence of the present disclosure may have at least 85% (e.g., at least 86%, 90%, 95%, 96%, 97%, 98%, 99%, or more) sequence identity to the nucleic acid sequence of SEQ ID NO: 90. For example, the ASO may have at least 90% (e.g., at least 90%, 95%, 96%, 97%, 98%, 99%, or more) sequence identity to the nucleic acid sequence of SEQ ID NO: 90. In another example, the ASO may have at least 95% (e.g., at least 95%, 96%, 97%, 98%, 99%, or more) sequence identity to the nucleic acid sequence of SEQ ID NO: 90. In a further example, the ASO may have the nucleic acid sequence of SEQ ID NO: 90.

An ASO sequence of the present disclosure may have at least 85% (e.g., at least 86%, 90%, 95%, 96%, 97%, 98%, 99%, or more) sequence identity to the nucleic acid sequence of SEQ ID NO: 91. For example, the ASO may have at least 90% (e.g., at least 90%, 95%, 96%, 97%, 98%, 99%, or more) sequence identity to the nucleic acid sequence of SEQ ID NO: 91. In another example, the ASO may have at least 95% (e.g., at least 95%, 96%, 97%, 98%, 99%, or more) sequence identity to the nucleic acid sequence of SEQ ID NO: 91. In a further example, the ASO may have the nucleic acid sequence of SEQ ID NO: 91.

An ASO sequence of the present disclosure may have at least 85% (e.g., at least 86%, 90%, 95%, 96%, 97%, 98%, 99%, or more) sequence identity to the nucleic acid sequence of SEQ ID NO: 92. For example, the ASO may have at least 90% (e.g., at least 90%, 95%, 96%, 97%, 98%, 99%, or more) sequence identity to the nucleic acid sequence of SEQ ID NO: 92. In another example, the ASO may have at least 95% (e.g., at least 95%, 96%, 97%, 98%, 99%, or more) sequence identity to the nucleic acid sequence of SEQ ID NO: 92. In a further example, the ASO may have the nucleic acid sequence of SEQ ID NO: 92.

An ASO sequence of the present disclosure may have at least 85% (e.g., at least 86%, 90%, 95%, 96%, 97%, 98%, 99%, or more) sequence identity to the nucleic acid sequence of SEQ ID NO: 93. For example, the ASO may have at least 90% (e.g., at least 90%, 95%, 96%, 97%, 98%, 99%, or more) sequence identity to the nucleic acid sequence of SEQ ID NO: 93. In another example, the ASO may have at least 95% (e.g., at least 95%, 96%, 97%, 98%, 99%, or more) sequence identity to the nucleic acid sequence of SEQ ID NO: 93. In a further example, the ASO may have the nucleic acid sequence of SEQ ID NO: 93.

An ASO sequence of the present disclosure may have at least 85% (e.g., at least 86%, 90%, 95%, 96%, 97%, 98%, 99%, or more) sequence identity to the nucleic acid sequence of SEQ ID NO: 94. For example, the ASO may have at least 90% (e.g., at least 90%, 95%, 96%, 97%, 98%, 99%, or more) sequence identity to the nucleic acid sequence of SEQ ID NO: 94. In another example, the ASO may have at least 95% (e.g., at least 95%, 96%, 97%, 98%, 99%, or more) sequence identity to the nucleic acid sequence of SEQ ID NO: 94. In a further example, the ASO may have the nucleic acid sequence of SEQ ID NO: 94.

An ASO sequence of the present disclosure may have at least 85% (e.g., at least 86%, 90%, 95%, 96%, 97%, 98%, 99%, or more) sequence identity to the nucleic acid sequence of SEQ ID NO: 95. For example, the ASO may have at least 90% (e.g., at least 90%, 95%, 96%, 97%, 98%, 99%, or more) sequence identity to the nucleic acid sequence of SEQ ID NO: 95. In another example, the ASO may have at least 95% (e.g., at least 95%, 96%, 97%, 98%, 99%, or more) sequence identity to the nucleic acid sequence of SEQ ID NO: 95. In a further example, the ASO may have the nucleic acid sequence of SEQ ID NO: 95.

An ASO sequence of the present disclosure may have at least 85% (e.g., at least 86%, 90%, 95%, 96%, 97%, 98%, 99%, or more) sequence identity to the nucleic acid sequence of SEQ ID NO: 96. For example, the ASO may have at least 90% (e.g., at least 90%, 95%, 96%, 97%, 98%, 99%, or more) sequence identity to the nucleic acid sequence of SEQ ID NO: 96. In another example, the ASO may have at least 95% (e.g., at least 95%, 96%, 97%, 98%, 99%, or more) sequence identity to the nucleic acid sequence of SEQ ID NO: 96. In a further example, the ASO may have the nucleic acid sequence of SEQ ID NO: 96.

An ASO sequence of the present disclosure may have at least 85% (e.g., at least 86%, 90%, 95%, 96%, 97%, 98%, 99%, or more) sequence identity to the nucleic acid sequence of SEQ ID NO: 97. For example, the ASO may have at least 90% (e.g., at least 90%, 95%, 96%, 97%, 98%, 99%, or more) sequence identity to the nucleic acid sequence of SEQ ID NO: 97. In another example, the ASO may have at least 95% (e.g., at least 95%, 96%, 97%, 98%, 99%, or more) sequence identity to the nucleic acid sequence of SEQ ID NO: 97. In a further example, the ASO may have the nucleic acid sequence of SEQ ID NO: 97.

An ASO sequence of the present disclosure may have at least 85% (e.g., at least 86%, 90%, 95%, 96%, 97%, 98%, 99%, or more) sequence identity to the nucleic acid sequence of SEQ ID NO: 98. For example, the ASO may have at least 90% (e.g., at least 90%, 95%, 96%, 97%, 98%, 99%, or more) sequence identity to the nucleic acid sequence of SEQ ID NO: 98. In another example, the ASO may have at least 95% (e.g., at least 95%, 96%, 97%, 98%, 99%, or more) sequence identity to the nucleic acid sequence of SEQ ID NO: 98. In a further example, the ASO may have the nucleic acid sequence of SEQ ID NO: 98.

An ASO sequence of the present disclosure may have at least 85% (e.g., at least 86%, 90%, 95%, 96%, 97%, 98%, 99%, or more) sequence identity to the nucleic acid sequence of SEQ ID NO: 99. For example, the ASO may have at least 90% (e.g., at least 90%, 95%, 96%, 97%, 98%, 99%, or more) sequence identity to the nucleic acid sequence of SEQ ID NO: 99. In another example, the ASO may have at least 95% (e.g., at least 95%, 96%, 97%, 98%, 99%, or more) sequence identity to the nucleic acid sequence of SEQ ID NO: 99. In a further example, the ASO may have the nucleic acid sequence of SEQ ID NO: 99.

An ASO sequence of the present disclosure may have at least 85% (e.g., at least 86%, 90%, 95%, 96%, 97%, 98%, 99%, or more) sequence identity to the nucleic acid sequence of SEQ ID NO: 100. For example, the ASO may have at least 90% (e.g., at least 90%, 95%, 96%, 97%, 98%, 99%, or more) sequence identity to the nucleic acid sequence of SEQ ID NO: 100. In another example, the ASO may have at least 95% (e.g., at least 95%, 96%, 97%, 98%, 99%, or more) sequence identity to the nucleic acid sequence of SEQ ID NO: 100. In a further example, the ASO may have the nucleic acid sequence of SEQ ID NO: 100.

Antisense Oligonucleotides with Wobble Base Pairs

The present disclosure further features ASO agents having one or more wobble base pairs. The four main wobble base pairs are guanine-uracil (G-U), hypoxanthine-uracil (I-U), hypoxanthine-adenine (I-A), and hypoxanthine-cytosine (I-C), in which hypoxanthine represents the nucleoside inosine. The G-U wobble base pair has been shown to exhibit a similar thermodynamic stability to that of G-C, A-T and A-U (Saxena et al, 2003, J Biol Chem, 278(45):44312-9).

Accordingly, the present disclosure provides an ASO agent having a nucleotide sequence that has at least 85% (e.g., at least 86%, 90%, 95%, 96%, 97%, 98%, 99%, or more) sequence identity to the complement of a target region of SEQ ID NO: 115 or SEQ ID NO: 116 (e.g., the ASO may have at least 85% sequence identity to the antisense strand of a Grik2 gene sequence). In particular, an ASO agent of the disclosure may have 1, 2 or 3 nucleotides that are not complementary to the corresponding aligned human Grik2 mRNA transcript (e.g., SEQ ID NO: 115 or SEQ ID NO: 116). As such, an ASO agent of the disclosure may have a nucleotide sequence that is at least 85% (e.g., at least 86%, 90%, 95%, 96%, 97%, 98%, 99%, or more), at least 86% (e.g., at least 86%, 90%, 95%, 96%, 97%, 98%, 99%, or more), at least 87% (e.g., at least 87%, 90%, 95%, 96%, 97%, 98%, 99%, or more), at least 88% (e.g., at least 88%, 90%, 95%, 96%, 97%, 98%, 99%, or more), at least 89% (e.g., at least 89%, 90%, 95%, 96%, 97%, 98%, 99%, or more) or at least 90% (e.g., at least 90%, 95%, 96%, 97%, 98%, 99%, or more) identical to the complement of a target region of SEQ ID NO: 116 or SEQ ID NO: 115. The nucleotides that are not 100% identical to the complementary sequence of the aligned Grik2 mRNA sequence may be a wobble nucleotide.

As shown in Table 2 herein, ASO agents with a lowercase ‘u’ in the 5′-end have one fewer nucleotide that is identical to the complementary sequence of the human Grik2 mRNA relative to the other human ASO agents listed in Table 2. The inclusion of ‘u’ at the 5′-end (resulting in a G:U wobble base pair) was implemented to improve RISC loading (siSPOTR software, Boudreau, R.L. et al., Nucleic Acid Res 2013, 41(1):e9).

The probability of off-target effects mediated by antisense RNAs designed against a particular region on a Grik2 transcript may be measured using any number of publicly available algorithms. For example, the online tool siSPOTR (“siRNA Sequence Probability-of-Off-Targeting Reduction”, which is available at world-wide-web.sispotr.icts.uiowa.edu/sispotr/index.html, can be used).

Certain Grik2 antisense sequences were determined to be “specific” siSPOTR guides (based on the off-target predictor program siSPOTR), and are antisense RNAs that have been predicted to avoid or reduce off-target sequence specific gene suppression in the human genome while maintaining sequence specific inhibition of transcripts including SEQ ID NO: 115 or SEQ ID NO: 116 (see Table 3).

Certain Grik2 antisense RNAs were determined to be “shared” siSPOTR sequences (based on the off-target predictor program siSPOTR), and are antisense RNAs that have been predicted to avoid or reduce off-target sequence specific gene suppression in the human genome and have significant shared homology between human, monkey and mouse Grik2 mRNA sequence, and are expected to maintain sequence specific inhibition of transcripts including SEQ ID NO: 115, SEQ ID NO: 116 (but also SEQ ID NOs: 117-125).

TABLE 3
siSPOTR-Predicted Grik2mRNA Antisense Sequences

| | | SEQ | | SEQ | | |
|---|---|---|---|---|---|---|
| | | ID | cDNA encoding target human | ID | Target sequence nucleotide | Grik2 mRNA target |
| ID | Predicted guide RNA (5′-3′) | NO | Grik2 mRNA sequence (5′-3′) | NO | position | region |
| TC | uGGUGCGCCUGAAGACUGGAU | 28 | ATCCAGTCTTCAGGCGCACCA | 609 | 29-48 (SEQ ID NO: 116) | Exon 1, |
| (siSPOTR1) | | | | | 322-341 (SEQ ID NO: 115) | Signal peptide |
| (specific) | | | | | | |
| CR | AGCGGGUCUGUAUGUGGGGAA | 36 | TTCCCCACATACAGACCCGCT | 617 | 380-400 (SEQ ID NO: 116) | Exon 3 |
| (siSPOTR2) | | | | | 673-693 (SEQ ID NO: 115) | |
| (specific) | | | | | | |
| G1 | AAUCGGGUUUCGGAGGUGCCU | 5 | AGGCACCTCCGAAACCCGATT | 586 | 905-925 (SEQ ID NO: 116) | Exon 6, Centroid |
| (siSPOTR3) | | | | | 1198-1218 (SEQ ID NO: 115) | loop 3 |
| (specific) | | | | | | |
| GG | AGUCGUCAUAAAUCCAUCCAG | 91 | CTGGATGGATTTATGACGACT | 673 | 934-954 (SEQ ID NO: 116) | Overlaps Exon 6 |
| (siSPOTR4) | | | | | 1227-1247 (SEQ ID NO: 115) | and Exon 7 |
| (shared and | | | | | | |
| specific) | | | | | | |
| TL | UGUCGAUUACACUGCAAGGAA | 20 | TTCCTTGCAGTGTAATCGACA | 601 | 1029-1049 (SEQ ID NO: 116) | Exon 7 |
| (siSPOTR5) | | | | | 1322-1342 (SEQ ID NO: 115) | |
| (specific) | | | | | | |
| Y8 | uAUAGGUAAUAGCCAGUGGAG | 87 | CTCCACTGGCTATTACCTAT | 669 | 1544-1563 (SEQ ID NO: 116) | Exon 11 |
| (siSPOTR6) | | | | | 1837-1856 (SEQ ID NO: 115) | |
| (shared) | | | | | | |
| TI | uUCGAACAUAGGUAAUAGCCA | 75 | TGGCTATTACCTATGTTCGA | 657 | 1550-1569 (SEQ ID NO: 116) | Exon 11 |
| (siSPOTR7) | | | | | 1843-1862 (SEQ ID NO: 115) | |
| (specific) | | | | | | |
| TK | AGUCGAUGACCUUCUCUCGAA | 74 | TTCGAGAGAAGGTCATCGACT | 656 | 1565-1585 (SEQ ID NO: 116) | Exon 11 |
| (siSPOTR8) | | | | | 1878-1898 (SEQ ID NO: 115) | |
| (specific) | | | | | | |
| CU | AGACGCCUGGGUUUGUACCAU | 39 | ATGGTACAAACCCAGGCGTCT | 620 | 1637-1657 (SEQ ID NO: 116) | Exon 11 |
| (siSPOTR9) | | | | | 1930-1950 (SEQ ID NO: 115) | |
| (shared and | | | | | | |
| specific) | | | | | | |
| Y7 | AGAUAUCAGGGGAGAGAGGAU | 62 | ATCCTCTCTCCCCTGATATCT | 643 | 1670-1690 (SEQ ID NO: 116) | Exon 11 |
| (siSPOTR10) | | | | | 1963-1983 (SEQ ID NO: 115) | |
| (shared) | | | | | | |
| XX | uACCACGUCUGAGUCAGGGUU | 82 | AACCCTGACTCAGACGTGGT | 664 | 1786-1805 (SEQ ID NO: 116) | Exon 12 |
| (siSPOTR11) | | | | | 2079-2098 (SEQ ID NO: 115) | |
| (shared) | | | | | | |
| CP | uUCGAUGGUUGUUGACUCCAU | 34 | ATGGAGTCAACAACCATCGA | 615 | 2209-2228 (SEQ ID NO: 116) | Exon 14 |
| (siSPOTR12) | | | | | 2502-2521 (SEQ ID NO: 115) | |
| (specific) | | | | | | |
| GI | GUCUCGAUAUGGAGAACCCAU | 77 | ATGGGTTCTCCATATCGAGAC | 659 | 2308-2328 (SEQ ID NO: 116) | Overlaps with Exon |
| (siSPOTR13) | | | | | 2601-2621 (SEQ ID NO: 115) | 13 and Exon 14 |
| (shared) | | | | | | |
| XU | UGUCUCGAUAUGGAGAACCCA | 51 | TGGGTTCTCCATATCGAGACA | 632 | 2309-2329 (SEQ ID NO: 116) | Exon 15 |
| (siSPOTR14) | | | | | 2602-2622 (SEQ ID NO: 115) | |
| (shared) | | | | | | |
| YB | uAUCCGGGAGAAAUCCAGCAC | 67 | GTGCTGGATTTCTCCCGGAT | 648 | 30-49 (SEQ ID NO: 115) | 5′ UTR |
| (siSPOTR15) | | | | | | Exon 1 |
| (specific) | | | | | | |
| ZZ | UAGUCGGAGAGCAUCCGGGAG | 100 | CTCCCGGATGCTCTCCGACTA | 682 | 41-61 (SEQ ID NO: 115) | 5′ UTR |
| (siSPOTR16) | | | | | | Exon 1 |
| (specific) | | | | | | |
| GE | UUAGUCGGAGAGCAUCCGGGA | 65 | TCCCGGATGCTCTCCGACTAA | 646 | 42-62 (SEQ ID NO: 115) | 5′ UTR |
| (siSPOTR17) | | | | | | Exon 1 |
| (specific) | | | | | | |
| D3 | GUCUCCGCUUCCCAAACCCAU | 48 | ATGGGTTTGGGAAGCGGAGAC | 636 | 139-159 (SEQ ID NO: 115) | 5′ UTR |
| (siSPOTR18) | | | | | | Exon 1 |
| (shared) | | | | | | |
| CX | GACCGCAGACACGAUCACGGC | 42 | GCCGTGATCGTGTCTGCGGTC | 630 | 182-202 (SEQ ID NO: 115) | 5′ UTR |
| (siSPOTR19) | | | | | | Exon 1 |
| (specific) | | | | | | |
| GF | UGCGUCGAGUGGUGACCGCAG | 64 | CTGCGGTCACCACTCGACGCA | 652 | 195-215 (SEQ ID NO: 115) | 5′ UTR |
| (siSPOTR20) | | | | | | Exon 1 |
| (specific) | | | | | | |
| GH | AUGCGUCGAGUGGUGACCGCA | 66 | TGCGGTCACCACTCGACGCAT | 654 | 196-216 (SEQ ID NO: 115) | 5′ UTR |
| (siSPOTR21) | | | | | | Exon 1 |
| (specific) | | | | | | |
| TJ | GAUGCGUCGAGUGGUGACCGC | 21 | GCGGTCACCACTCGACGCATC | 609 | 197-217 (SEQ ID NO: 115) | 5′ UTR |
| (siSPOTR22) | | | | | | Exon 1 |
| (specific) | | | | | | |
| TG | GGUUCGGGUAGAAAUGAGGAU | 23 | ATCCTCATTTCTACCCGAACC | 611 | 215-235 (SEQ ID NO: 115) | 5′ UTR |
| (siSPOTR23) | | | | | | Exon 1 |
| (specific) | | | | | | |
| TD | ACCCGAACCCAGGAGCCGAAC | 26 | ACCCGAACCCAGGAGCCGAA | 614 | 227-247 (SEQ ID NO: 115) | 5′ UTR |
| (siSPOTR24) | | | C | | | Exon 1 |
| (specific) | | | | | | |
| TF | uUAGCGUUCGGCUCCUGGGUU | 24 | AACCCAGGAGCCGAACGCTA | 612 | 232-251 (SEQ ID NO: 115) | 5′ UTR |
| (siSPOTR25) | | | | | | Exon 1 |
| (specific) | | | | | | |

The ASO agents disclosed herein target an mRNA encoding a GluK2 protein (e.g., GluK2 protein including any one of SEQ ID NOs: 102-114, or GluK2 protein including at least amino acids 1 to 509 of SEQ ID NO: 102). The mRNA encoding a GluK2 protein may include a polynucleotide encoding polypeptide that contains one or more amino acid substitutions, such as one or more conservative amino acid substitutions (e.g., 1, 2, 3, 4, 5, 6, 7, 8, 9, or or more amino acid substitutions, such as 1, 2, 3, 4, 5, 6, 7, 8, 9, or or more conservative amino acid substitutions), relative to a polypeptide having the sequence of any one of SEQ ID NOs: 102-114.

The Grik2 ASO agents disclosed herein may be designed by using the sequence of the Grik2 mRNA as a starting point by using, e.g., bioinformatic tools. Grik2 mRNA sequences may be found in NCBI Gene ID NO: 2898. In another example, a polynucleotide sequence encoding SEQ ID NO: 102, a polynucleotide sequence encoding contiguous amino acids 1 to 509 of SEQ ID NO: 102, or a polynucleotide sequence encoding the amino acid sequence of any one of SEQ ID NO: 102 (UniProtKB Q13002-1), SEQ ID NO: 103 (UniProtKB Q13002-2), SEQ ID NO: 104 (UniProtKB Q13002-3), SEQ ID NO: 105 (UniProtKB Q13002-4), SEQ ID NO: 106 (UniProtKB Q13002-5), SEQ ID NO: 107 (UniProtKB Q13002-6), SEQ ID NO: 108 (UniProtKB Q13002-7), SEQ ID NO: 109 (NCBI Accession No.: NP_001104738.2), SEQ ID NO: 110 (NCBI Accession No.: NP_034479.3), SEQ ID NO: 111 (NCBI Accession No.: NP_034479.3), SEQ ID NO: 112 (NCBI Accession No.: XP_014992481.1), SEQ ID NO: 113 (NCBI Accession No.: XP_014992483.1), and SEQ ID NO: 114 (NCBI Accession No.: NP_062182.1) can be used as a basis for designing nucleic acids that target an mRNA encoding GluK2 protein. Polynucleotide sequences encoding a GluK2 receptor may be selected from any one of SEQ ID NOs: 115-125.

The GluK2 polypeptide may have an amino acid sequence of SEQ ID NO: 102 or may be a variant thereof with at least 85% (e.g., at least 86%, 90%, 95%, 96%, 97%, 98%, 99%, or more) sequence identity to the amino acid sequence of SEQ ID NO: 102, which is shown below (UniProt Q13002-1; GRIK2_HUMAN Glutamate receptor ionotropic, kainate 2):

(SEQ ID NO: 102)
MKIIFPILSNPVFRRTVKLLLCLLWIGYSQGTTHVLRFGGIFEYVESGP
MGAEELAFRFAVNTINRNRTLLPNTTLTYDTOKINLYDSFEASKKACDQ
LSLGVAAIFGPSHSSSANAVQSICNALGVPHIQTRWKHQVSDNKDSFYV
SLYPDFSSLSRAILDLVQFFKWKTVTVVYDDSTGLIRLQELIKAPSRYN
LRLKIRQLPADTKDAKPLLKEMKRGKEFHVIFDCSHEMAAGILKQALAM
GMMTEYYHYIFTTLDLFALDVEPYRYSGVNMTGFRILNTENTQVSSIIE
KWSMERLQAPPKPDSGLLDGFMTTDAALMYDAVHVVSVAVQQFPQMTVS
SLQCNRHKPWRFGTRFMSLIKEAHWEGLTGRITFNKTNGLRTDFDLDVI
SLKEEGLEKIGTWDPASGLNMTESQKGKPANITDSLSNRSLIVTTILEE
PYVLFKKSDKPLYGNDRFEGYCIDLLRELSTILGFTYEIRLVEDGKYGA
QDDANGQWNGMVRELIDHKADLAVAPLAITYVREKVIDFSKPFMTLGIS
ILYRKPNGTNPGVFSFLNPLSPDIWMYILLAYLGVSCVLFVIARFSPYE
WYNPHPCNPDSDVVENNFTLLNSFWFGVGALMQQGSELMPKALSTRIVG
GIWWFFTLIIISSYTANLAAFLTVERMESPIDSADDLAKQTKIEYGAVE
DGATMTFFKKSKISTYDKMWAFMSSRRQSVLVKSNEEGIQRVLTSDYAF
LMESTTIEFVTQRNCNLTQIGGLIDSKGYGVGTPMGSPYRDKITIAILQ
LQEEGKLHMMKEKWWRGNGCPEEESKEASALGVQNIGGIFIVLAAGLVL
SVFVAVGEFLYKSKKNAQLEKRSFCSAMVEELRMSLKCQRRLKHKPQAP
VIVKTEEVINMHTFNDRRLPGKETMA

The GluK2 polypeptide may have an amino acid sequence of SEQ ID NO: 103 or may be a variant thereof with at least 85% (e.g., at least 86%, 90%, 95%, 96%, 97%, 98%, 99%, or more) sequence identity to the amino acid sequence of SEQ ID NO: 103, which is shown below (UniProt Q13002-2; GRIK2_HUMAN Isoform 2 of Glutamate receptor ionotropic, kainate 2):

(SEQ ID NO: 103)
MKIIFPILSNPVFRRTVKLLLCLLWIGYSQGTTHVLRFGGIFEYVESGP
MGAEELAFRFAVNTINRNRTLLPNTTLTYDTOKINLYDSFEASKKACDQ
LSLGVAAIFGPSHSSSANAVQSICNALGVPHIQTRWKHQVSDNKDSFYV
SLYPDFSSLSRAILDLVQFFKWKTVTVVYDDSTGLIRLQELIKAPSRYN
LRLKIRQLPADTKDAKPLLKEMKRGKEFHVIFDCSHEMAAGILKQALAM
GMMTEYYHYIFTTLDLFALDVEPYRYSGVNMTGFRILNTENTQVSSIIE
KWSMERLQAPPKPDSGLLDGFMTTDAALMYDAVHVVSVAVQQFPQMTVS
SLQCNRHKPWRFGTRFMSLIKEAHWEGLTGRITFNKTNGLRTDFDLDVI
SLKEEGLEKIGTWDPASGLNMTESQKGKPANITDSLSNRSLIVTTILEE
PYVLFKKSDKPLYGNDRFEGYCIDLLRELSTILGFTYEIRLVEDGKYGA
QDDANGQWNGMVRELIDHKADLAVAPLAITYVREKVIDFSKPFMTLGIS
ILYRKPNGTNPGVFSFLNPLSPDIWMYILLAYLGVSCVLFVIARFSPYE
WYNPHPCNPDSDVVENNFTLLNSFWFGVGALMQQGSELMPKALSTRIVG
GIWWFFTLIIISSYTANLAAFLTVERMESPIDSADDLAKQTKIEYGAVE
DGATMTFFKKSKISTYDKMWAFMSSRRQSVLVKSNEEGIQRVLTSDYAF
LMESTTIEFVTQRNCNLTQIGGLIDSKGYGVGTPMGSPYRDKITIAILQ
LQEEGKLHMMKEKWWRGNGCPEEESKEASALGVQNIGGIFIVLAAGLVL
SVFVAVGEFLYKSKKNAQLEKESSIWLVPPYHPDTV

The GluK2 polypeptide may have an amino acid sequence of SEQ ID NO: 104 or may be a variant thereof with at least 85% (e.g., at least 86%, 90%, 95%, 96%, 97%, 98%, 99%, or more) sequence identity to the amino acid sequence of SEQ ID NO: 104, which is shown below (UniProt Q13002-3; GRIK2_HUMAN Isoform 3 of Glutamate receptor ionotropic, kainate 2):

(SEQ ID NO: 104)
MKIIFPILSNPVFRRTVKLLLCLLWIGYSQGTTHVLRFGGIFEYVESGP
MGAEELAFRFAVNTINRNRTLLPNTTLTYDTOKINLYDSFEASKKACDQ
LSLGVAAIFGPSHSSSANAVQSICNALGVPHIQTRWKHQVSDNKDSFYV
SLYPDFSSLSRAILDLVQFFKWKTVTVVYDDSTGLIRLQELIKAPSRYN
LRLKIRQLPADTKDAKPLLKEMKRGKEFHVIFDCSHEMAAGILKQALAM
GMMTEYYHYIFTTLDLFALDVEPYRYSGVNMTGFRILNTENTQVSSIIE
KWSMERLQAPPKPDSGLLDGFMTTDAALMYDAVHVVSVAVQQFPQMTVS
SLQCNRHKPWRFGTRFMSLIKEAHWEGLTGRITFNKTNGLRTDFDLDVI
SLKEEGLEKIGTWDPASGLNMTESQKGKPANITDSLSNRSLIVTTILEE
PYVLFKKSDKPLYGNDRFEGYCIDLLRELSTILGFTYEIRLVEDGKYGA
QDDANGQWNGMVRELIDHKADLAVAPLAITYVREKVIDFSKPFMTLGIS
ILYRKPNGTNPGVFSFLNPLSPDIWMYILLAYLGVSCVLFVIARF

The GluK2 polypeptide may have an amino acid sequence of SEQ ID NO: 105 or may be a variant thereof with at least 85% (e.g., at least 86%, 90%, 95%, 96%, 97%, 98%, 99%, or more) sequence identity to the amino acid sequence of SEQ ID NO: 105, which is shown below (UniProt Q13002-4; GRIK2_HUMAN Isoform 4 of Glutamate receptor ionotropic, kainate 2):

(SEQ ID NO: 105)
MKIIFPILSNPVFRRTVKLLLCLLWIGYSQGTTHVLRFGGIFEYVESGP
MGAEELAFRFAVNTINRNRTLLPNTTLTYDTOKINLYDSFEASKKACDQ
LSLGVAAIFGPSHSSSANAVQSICNALGVPHIQTRWKHQVSDNKDSFYV
SLYPDFSSLSRAILDLVQFFKWKTVTVVYDDSTGLIRLQELIKAPSRYN
LRLKIRQLPADTKDAKPLLKEMKRGKEFHVIFDCSHEMAAGILKQALAM
GMMTEYYHYIFTTLDLFALDVEPYRYSGVNMTGFRILNTENTQVSSIIE
KWSMERLQAPPKPDSGLLDGFMTTDAALMYDAVHVVSVAVQQFPQMTVS
SLQCNRHKPWRFGTRFMSLIKEAHWEGLTGRITFNKTNGLRTDFDLDVI
SLKEEGLEKIGTWDPASGLNMTESQKGKPANITDSLSNRSLIVTTILEE
PYVLFKKSDKPLYGNDRFEGYCIDLLRELSTILGFTYEIRLVEDGKYGA
QDDANGQWNGMVRELIDHKADLAVAPLAITYVREKVIDFSKPFMTLGIS
ILYRKPNGSELMPKALSTRIVGGIWWFFTLIIISSYTANLAAFLTVERM
ESPIDSADDLAKQTKIEYGAVEDGATMTFFKKSKISTYDKMWAFMSSRR
QSVLVKSNEEGIQRVLTSDYAFLMESTTIEFVTQRNCNLTQIGGLIDSK
GYGVGTPMGSPYRDKITIAILQLQEEGKLHMMKEKWWRGNGCPEEESKE
ASALGVQNIGGIFIVLAAGLVLSVFVAVGEFLYKSKKNAQLEKRSFCSA
MVEELRMSLKCQRRLKHKPQAPVIVKTEEVINMHTFNDRRLPGKETMA

The GluK2 polypeptide may have an amino acid sequence of SEQ ID NO: 106 or may be a variant thereof with at least 85% (e.g., at least 86%, 90%, 95%, 96%, 97%, 98%, 99%, or more) sequence identity to the amino acid sequence of SEQ ID NO: 106, which is shown below (UniProt Q13002-5; GRIK2_HUMAN Isoform of Glutamate receptor ionotropic, kainate 2):

(SEQ ID NO: 106)
MKIIFPILSNPVFRRTVKLLLCLLWIGYSQGTTHVLRFGGIFEYVESGP
MGAEELAFRFAVNTINRNRTLLPNTTLTYDTOKINLYDSFEASKKACDQ
LSLGVAAIFGPSHSSSANAVQSICNALGVPHIQTRWKHQVSDNKDSFYV
SLYPDFSSLSRAILDLVQFFKWKTVTVVYDDSTGLIRLQELIKAPSRYN
LRLKIRQLPADTKDAKPLLKEMKRGKEFHVIFDCSHEMAAGILKQALAM
GMMTEYYHYIFTTLDLFALDVEPYRYSGVNMTGFRILNTENTQVSSIIE
KWSMERLQAPPKPDSGLLDGFMTTDAALMYDAVHVVSVAVQQFPQMTVS
SLQCNRHKPWRFGTRFMSLIKEAHWEGLTGRITFNKTNGLRTDFDLDVI
SLKEEGLEKIGTWDPASGLNMTESQKGKPANITDSLSNRSLIVTTILEE
PYVLFKKSDKPLYGNDRFEGYCIDLLRELSTILGFTYEIRLVEDGKYGA
QDDANGQWNGMVRELIDHKADLAVAPLAITYVREKVIDFSKPFMTLGIS
ILYRKPNGTNPGVFSFLNPLSPDIWMYILLAYLGVSCVLFVIARFSPYE
WYNPHPCNPDSDVVENNFTLLNSFWFGVGALMQQGSELMPKALSTRIVG
GIWWFFTLIIISSYTANLAAFLTVERMESPIDSADDLAKQTKIEYGAVE
DGATMTFFKKSKISTYDKMWAFMSSRRQSVLVKSNEEGIQRVLTSDYAF
LMESTTIEFVTQRNCNLTQIGGLIDSKGYGVGTPMGSPYRDKITIAILQ
LQEEGKLHMMKEKWWRGNGCPEEESKEASALGVQNIGGIFIVLAAGLVL
SVFVAVGEFLYKSKKNAQLEKRAKTKLPQDYVFLPILESVSISTVLSSS
PSSSSLSSCS

The GluK2 polypeptide may have an amino acid sequence of SEQ ID NO: 107 or may be a variant thereof with at least 85% (e.g., at least 86%, 90%, 95%, 96%, 97%, 98%, 99%, or more) sequence identity to the amino acid sequence of SEQ ID NO: 107, which is shown below (UniProt Q13002-6; GRIK2_HUMAN Isoform 6 of Glutamate receptor ionotropic, kainate 2):

(SEQ ID NO: 107)
MKIIFPILSNPVFRRTVKLLLCLLWIGYSQGTTHVLRFGGIFEYVESGP
MGAEELAFRFAVNTINRNRTLLPNTTLTYDTOKINLYDSFEASKKACDQ
LSLGVAAIFGPSHSSSANAVQSICNALGVPHIQTRWKHQVSDNKDSFYV
SLYPDFSSLSRAILDLVQFFKWKTVTVVYDDSTGLIRLQELIKAPSRYN
LRLKIRQLPADTKDAKPLLKEMKRGKEFHVIFDCSHEMAAGILKQALAM
GMMTEYYHYIFTTLDLFALDVEPYRYSGVNMTGFRILNTENTQVSSIIE
KWSMERLQAPPKPDSGLLDGFMTTDAALMYDAVHVVSVAVQQFPQMTVS
SLQCNRHKPWRFGTRFMSLIKEAHWEGLTGRITFNKTNGLRTDFDLDVI
SLKEEGLEKIGTWDPASGLNMTESQKGKPANITDSLSNRSLIVTTILEE
PYVLFKKSDKPLYGNDRFEGYCIDLLRELSTILGFTYEIRLVEDGKYGA
QDDANGQWNGMVRELIDHKSKISTYDKMWAFMSSRRQSVLVKSNEEGIQ
RVLTSDYAFLMESTTIEFVTQRNCNLTQIGGLIDSKGYGVGTPMGSPYR
DKITIAILQLQEEGKLHMMKEKWWRGNGCPEEESKEASALGVQNIGGIF
IVLAAGLVLSVFVAVGEFLYKSKKNAQLEKESSIWLVPPYHPDTV

The GluK2 polypeptide may have an amino acid sequence of SEQ ID NO: 108 or may be a variant thereof with at least 85% (e.g., at least 86%, 90%, 95%, 96%, 97%, 98%, 99%, or more) sequence identity to the amino acid sequence of SEQ ID NO: 108, which is shown below (UniProt Q13002-7; GRIK2_HUMAN Isoform 7 of Glutamate receptor ionotropic, kainate 2):

(SEQ ID NO: 108)
MKIIFPILSNPVFRRTVKLLLCLLWIGYSQGTTHVLRFGGIFEYVESGP
MGAEELAFRFAVNTINRNRTLLPNTTLTYDTOKINLYDSFEASKKACDQ
LSLGVAAIFGPSHSSSANAVQSICNALGVPHIQTRWKHQVSDNKDSFYV
SLYPDFSSLSRAILDLVQFFKWKTVTVVYDDSTGLIRLQELIKAPSRYN
LRLKIRQLPADTKDAKPLLKEMKRGKEFHVIFDCSHEMAAGILKQALAM
GMMTEYYHYIFTTLDLFALDVEPYRYSGVNMTGFRILNTENTQVSSIIE
KWSMERLQAPPKPDSGLLDGFMTTDAALMYDAVHVVSVAVQQFPQMTVS
SLQCNRHKPWRFGTRFMSLIKEAHWEGLTGRITFNKTNGLRTDFDLDVI
SLKEEGLEKIGTWDPASGLNMTESQKGKPANITDSLSNRSLIVTTILEE
PYVLFKKSDKPLYGNDRFEGYCIDLLRELSTILGFTYEIRLVEDGKYGA
QDDANGQWNGMVRELIDHKSVLVKSNEEGIQRVLTSDYAFLMESTTIEF
VTQRNCNLTQIGGLIDSKGYGVGTPMGSPYRDKITIAILQLQEEGKLHM
MKEKWWRGNGCPEEESKEASALGVQNIGGIFIVLAAGLVLSVFVAVGEF
LYKSKKNAQLEKRAKTKLPQDYVFLPILESVSISTVLSSSPSSSSLSSC
S

The GluK2 polypeptide may have an amino acid sequence of SEQ ID NO: 109 or may be a variant thereof with at least 85% (e.g., at least 86%, 90%, 95%, 96%, 97%, 98%, 99%, or more) sequence identity to the amino acid sequence of SEQ ID NO: 109, which is shown below (NP_001104738.2; GRIK2_MOUSE Isoform 1 precursor of Glutamate receptor ionotropic, kainate 2):

(SEQ ID NO: 109)
MKIISPVLSNLVFSRSIKVLLCLLWIGYSQGTTHVLRFGGIFEYVESGPM
GAEELAFRFAVNTINRNRTLLPNTTLTYDTQKINLYDSFEASKKACDQLS
LGVAAIFGPSHSSSANAVQSICNALGVPHIQTRWKHQVSDNKDSFYVSLY
PDFSSLSRAILDLVQFFKWKTVTVVYDDSTGLIRLQELIKAPSRYNLRLK
IRQLPADTKDAKPLLKEMKRGKEFHVIFDCSHEMAAGILKQALAMGMMTE
YYHYIFTTLDLFALDVEPYRYSGVNMTGFRILNTENTQVSSIIEKWSMER
LQAPPKPDSGLLDGFMTTDAALMYDAVHVVSVAVQQFPQMTVSSLQCNRH
KPWRFGTRFMSLIKEAHWEGLTGRITFNKTNGLRTDFDLDVISLKEEGLE
KIGTWDPSSGLNMTESQKGKPANITDSLSNRSLIVTTILEEPYVLFKKSD
KPLYGNDRFEGYCIDLLRELSTILGFTYEIRLVEDGKYGAQDDVNGQWNG
MVRELIDHKADLAVAPLAITYVREKVIDFSKPFMTLGISILYRKPNGTNP
GVFSFLNPLSPDIWMYILLAYLGVSCVLFVIARFSPYEWYNPHPCNPDSD
VVENNFTLLNSFWFGVGALMQQGSELMPKALSTRIVGGIWWFFTLIIISS
YTANLAAFLTVERMESPIDSADDLAKQTKIEYGAVEDGATMTFFKKSKIS
TYDKMWAFMSSRRQSVLVKSNEEGIQRVLTSDYAFLMESTTIEFVTQRNC
NLTQIGGLIDSKGYGVGTPMGSPYRDKITIAILQLQEEGKLHMMKEKWWR
GNGCPEEESKEASALGVQNIGGIFIVLAAGLVLSVFVAVGEFLYKSKKNA
QLEKRSFCSAMVEELRMSLKCQRRLKHKPQAPVIVKTEEVINMHTFNDRR
LPGKETMA

The GluK2 polypeptide may have an amino acid sequence of SEQ ID NO: 110 or may be a variant thereof with at least 85% (e.g., at least 86%, 90%, 95%, 96%, 97%, 98%, 99%, or more) sequence identity to the amino acid sequence of SEQ ID NO: 110, which is shown below (NP_034479.3; GRIK2_MOUSE Isoform 2 precursor of Glutamate receptor ionotropic, kainate 2):

(SEQ ID NO: 110)
MKIISPVLSNLVFSRSIKVLLCLLWIGYSQGTTHVLRFGGIFEYVESGPM
GAEELAFRFAVNTINRNRTLLPNTTLTYDTQKINLYDSFEASKKACDQLS
LGVAAIFGPSHSSSANAVQSICNALGVPHIQTRWKHQVSDNKDSFYVSLY
PDFSSLSRAILDLVQFFKWKTVTVVYDDSTGLIRLQELIKAPSRYNLRLK
IRQLPADTKDAKPLLKEMKRGKEFHVIFDCSHEMAAGILKQALAMGMMTE
YYHYIFTTLDLFALDVEPYRYSGVNMTGFRILNTENTQVSSIIEKWSMER
LQAPPKPDSGLLDGFMTTDAALMYDAVHVVSVAVQQFPQMTVSSLQCNRH
KPWRFGTRFMSLIKEAHWEGLTGRITFNKTNGLRTDFDLDVISLKEEGLE
KIGTWDPSSGLNMTESQKGKPANITDSLSNRSLIVTTILEEPYVLFKKSD
KPLYGNDRFEGYCIDLLRELSTILGFTYEIRLVEDGKYGAQDDVNGQWNG
MVRELIDHKADLAVAPLAITYVREKVIDFSKPFMTLGISILYRKPNGTNP
GVFSFLNPLSPDIWMYILLAYLGVSCVLFVIARFSPYEWYNPHPCNPDSD
VVENNFTLLNSFWFGVGALMQQGSELMPKALSTRIVGGIWWFFTLIIISS
YTANLAAFLTVERMESPIDSADDLAKQTKIEYGAVEDGATMTFFKKSKIS
TYDKMWAFMSSRRQSVLVKSNEEGIQRVLTSDYAFLMESTTIEFVTQRNC
NLTQIGGLIDSKGYGVGTPMGSPYRDKITIAILQLQEEGKLHMMKEKWWR
GNGCPEEESKEASALGVQNIGGIFIVLAAGLVLSVFVAVGEFLYKSKKNA
QLEKESSIWLVPPYHPDTV

The GluK2 polypeptide may have an amino acid sequence of SEQ ID NO: 111 or may be a variant thereof with at least 85% (e.g., at least 86%, 90%, 95%, 96%, 97%, 98%, 99%, or more) sequence identity to the amino acid sequence of SEQ ID NO: 111, which is shown below (NP_001345795.2; GRIK2_MOUSE Isoform 1 precursor of Glutamate receptor ionotropic, kainate 2):

(SEQ ID NO: 111)
MKIISPVLSNLVFSRSIKVLLCLLWIGYSQGTTHVLRFGGIFEYVESGPM
GAEELAFRFAVNTINRNRTLLPNTTLTYDTQKINLYDSFEASKKACDQLS
LGVAAIFGPSHSSSANAVQSICNALGVPHIQTRWKHQVSDNKDSFYVSLY
PDFSSLSRAILDLVQFFKWKTVTVVYDDSTGLIRLQELIKAPSRYNLRLK
IRQLPADTKDAKPLLKEMKRGKEFHVIFDCSHEMAAGILKQALAMGMMTE
YYHYIFTTLDLFALDVEPYRYSGVNMTGFRILNTENTQVSSIIEKWSMER
LQAPPKPDSGLLDGFMTTDAALMYDAVHVVSVAVQQFPQMTVSSLQCNRH
KPWRFGTRFMSLIKEAHWEGLTGRITFNKTNGLRTDFDLDVISLKEEGLE
KIGTWDPSSGLNMTESQKGKPANITDSLSNRSLIVTTILEEPYVLFKKSD
KPLYGNDRFEGYCIDLLRELSTILGFTYEIRLVEDGKYGAQDDVNGQWNG
MVRELIDHKADLAVAPLAITYVREKVIDFSKPFMTLGISILYRKPNGTNP
GVFSFLNPLSPDIWMYILLAYLGVSCVLFVIARFSPYEWYNPHPCNPDSD
VVENNFTLLNSFWFGVGALMQQGSELMPKALSTRIVGGIWWFFTLIIISS
YTANLAAFLTVERMESPIDSADDLAKQTKIEYGAVEDGATMTFFKKSKIS
TYDKMWAFMSSRRQSVLVKSNEEGIQRVLTSDYAFLMESTTIEFVTQRNC
NLTQIGGLIDSKGYGVGTPMGSPYRDKITIAILQLQEEGKLHMMKEKWWR
GNGCPEEESKEASALGVQNIGGIFIVLAAGLVLSVFVAVGEFLYKSKKNA
QLEKRSFCSAMVEELRMSLKCQRRLKHKPQAPVIVKTEEVINMHTFNDRR
LPGKETMA

The GluK2 polypeptide may have an amino acid sequence of SEQ ID NO: 112 or may be a variant thereof with at least 85% (e.g., at least 86%, 90%, 95%, 96%, 97%, 98%, 99%, or more) sequence identity to the amino acid sequence of SEQ ID NO: 112, which is shown below (XP_014992481.1; GRIK2_RHESUS MACAQUE Isoform X1, Glutamate receptor ionotropic, kainate 2):

(SEQ ID NO: 112)
MKIIFPILSNPVFRRTVKLLLCLLWIGYSQGTTHVLRFGGIFEYVESGPM
GAEELAFRFAVNTINRNRTLLPNTTLTYDTQKINLYDSFEASKKACDQLS
LGVAAIFGPSHSSSANAVQSICNALGVPHIQTRWKHQVSDNKDSFYVSLY
PDFSSLSRAILDLVQFFKWKTVTVVYDDSTGLIRLQELIKAPSRYNLRLK
IRQLPADTKDAKPLLKEMKRGKEFHVIFDCSHEMAAGILKQALAMGMMTE
YYHYIFTTLDLFALDVEPYRYSGVNMTGFRILNTENTQVSSIIEKWSMER
LQAPPKPDSGLLDGFMTTDAALMYDAVHVVSVAVQQFPQMTVSSLQCNRH
KPWRFGTRFMSLIKEAHWEGLTGRITFNKTNGLRTDFDLDVISLKEEGLE
KIGTWDPASGLNMTESQKGKPANITDSLSNRSLIVTTILEEPYVLFKKSD
KPLYGNDRFEGYCIDLLRELSTILGFTYEIRLVEDGKYGAQDDANGQWNG
MVRELIDHKADLAVAPLAITYVREKVIDFSKPFMTLGISILYRKPNGTNP
GVFSFLNPLSPDIWMYILLAYLGVSCVLFVIARFSPYEWYNPHPCNPDSD
VVENNFTLLNSFWFGVGALMQQGSELMPKALSTRIVGGIWWFFTLIIISS
YTANLAAFLTVERMESPIDSADDLAKQTKIEYGAVEDGATMTFFKKSKIS
TYDKMWAFMSSRRQSVLVKSNEEGIQRVLTSDYAFLMESTTIEFVTQRNC
NLTQIGGLIDSKGYGVGTPMGSPYRDKITIAILQLQEEGKLHMMKEKWWR
GNGCPEEESKEASALGVQNIGGIFIVLAAGLVLSVFVAVGEFLYKSKKNA
QLEKRSFCSAMVEELRMSLKCQRRLKHKPQAPVIVKTEEVINMHTFNDRR
LPGKETMA

The GluK2 polypeptide may have an amino acid sequence of SEQ ID NO: 113 or may be a variant thereof with at least 85% (e.g., at least 86%, 90%, 95%, 96%, 97%, 98%, 99%, or more) sequence identity to the amino acid sequence of SEQ ID NO: 113, which is shown below (XP_014992483.1; GRIK2_RHESUS MACAQUE Isoform X1, Glutamate receptor ionotropic, kainate 2):

(SEQ ID NO: 113)
MKIIFPILSNPVFRRTVKLLLCLLWIGYSQGTTHVLRFGGIFEYVESGPM
GAEELAFRFAVNTINRNRTLLPNTTLTYDTQKINLYDSFEASKKACDQLS
LGVAAIFGPSHSSSANAVQSICNALGVPHIQTRWKHQVSDNKDSFYVSLY
PDFSSLSRAILDLVQFFKWKTVTVVYDDSTGLIRLQELIKAPSRYNLRLK
IRQLPADTKDAKPLLKEMKRGKEFHVIFDCSHEMAAGILKQALAMGMMTE
YYHYIFTTLDLFALDVEPYRYSGVNMTGFRILNTENTQVSSIIEKWSMER
LQAPPKPDSGLLDGFMTTDAALMYDAVHVVSVAVQQFPQMTVSSLQCNRH
KPWRFGTRFMSLIKEAHWEGLTGRITFNKTNGLRTDFDLDVISLKEEGLE
KIGTWDPASGLNMTESQKGKPANITDSLSNRSLIVTTILEEPYVLFKKSD
KPLYGNDRFEGYCIDLLRELSTILGFTYEIRLVEDGKYGAQDDANGQWNG
MVRELIDHKADLAVAPLAITYVREKVIDFSKPFMTLGISILYRKPNGTNP
GVFSFLNPLSPDIWMYILLAYLGVSCVLFVIARFSPYEWYNPHPCNPDSD
VVENNFTLLNSFWFGVGALMQQGSELMPKALSTRIVGGIWWFFTLIIISS
YTANLAAFLTVERMESPIDSADDLAKQTKIEYGAVEDGATMTFFKKSKIS
TYDKMWAFMSSRRQSVLVKSNEEGIQRVLTSDYAFLMESTTIEFVTQRNC
NLTQIGGLIDSKGYGVGTPMGSPYRDKITIAILQLQEEGKLHMMKEKWWR
GNGCPEEESKEASALGVQNIGGIFIVLAAGLVLSVFVAVGEFLYKSKKNA
QLEKRSFCSAMVEELRMSLKCQRRLKHKPQAPVIVKTEEVINMHTFNDRR
LPGKETMA

The GluK2 polypeptide may have an amino acid sequence of SEQ ID NO: 114 or may be a variant thereof with at least 85% (e.g., at least 86%, 90%, 95%, 96%, 97%, 98%, 99%, or more) sequence identity to the amino acid sequence of SEQ ID NO: 114, which is shown below (NP_062182.1; GRIK2_RAT precursor of Glutamate receptor ionotropic, kainate 2):

(SEQ ID NO: 114)
MKIISPVLSNLVFSRSIKVLLCLLWIGYSQGTTHVLRFGGIFEYVESGPM
GAEELAFRFAVNTINRNRTLLPNTTLTYDTQKINLYDSFEASKKACDQLS
LGVAAIFGPSHSSSANAVQSICNALGVPHIQTRWKHQVSDNKDSFYVSLY
PDFSSLSRAILDLVQFFKWKTVTVVYDDSTGLIRLQELIKAPSRYNLRLK
IRQLPADTKDAKPLLKEMKRGKEFHVIFDCSHEMAAGILKQALAMGMMTE
YYHYIFTTLDLFALDVEPYRYSGVNMTGFRILNTENTQVSSIIEKWSMER
LQAPPKPDSGLLDGFMTTDAALMYDAVHVVSVAVQQFPQMTVSSLQCNRH
KPWRFGTRFMSLIKEAHWEGLTGRITFNKTNGLRTDFDLDVISLKEEGLE
KIGTWDPASGLNMTESQKGKPANITDSLSNRSLIVTTILEEPYVLFKKSD
KPLYGNDRFEGYCIDLLRELSTILGFTYEIRLVEDGKYGAQDDVNGQWNG
MVRELIDHKADLAVAPLAITYVREKVIDFSKPFMTLGISILYRKPNGTNP
GVFSFLNPLSPDIWMYVLLACLGVSCVLFVIARFSPYEWYNPHPCNPDSD
VVENNFTLLNSFWFGVGALMRQGSELMPKALSTRIVGGIWWFFTLIIISS
YTANLAAFLTVERMESPIDSADDLAKQTKIEYGAVEDGATMTFFKKSKIS
TYDKMWAFMSSRRQSVLVKSNEEGIQRVLTSDYAFLMESTTIEFVTQRNC
NLTQIGGLIDSKGYGVGTPMGSPYRDKITIAILQLQEEGKLHMMKEKWWR
GNGCPEEESKEASALGVQNIGGIFIVLAAGLVLSVFVAVGEFLYKSKKNA
QLEKRSFCSAMVEELRMSLKCQRRLKHKPQAPVIVKTEEVINMHTFNDRR
LPGKETMA

The Grik2 mRNA may be a polynucleotide containing 5′ and a 3′ untranslated regions (UTR) and having a nucleic acid sequence of SEQ ID NO: 115 or may be a variant thereof having at least 85% (e.g., at least 86%, 90%, 95%, 96%, 97%, 98%, 99%, or more) sequence identity to the nucleic acid sequence of SEQ ID NO: 115 (RefSeq NM_021956.1:4592 Homo sapiens glutamate ionotropic receptor kainate type subunit 2 (GRIK2), transcript variant 1, mRNA), as is shown in Table 4.

The Grik2 mRNA may be a polynucleotide having a nucleic acid sequence of SEQ ID NO: 116 or may be a variant thereof having at least 85% (e.g., at least 86%, 90%, 95%, 96%, 97%, 98%, 99%, or more) sequence identity to the nucleic acid sequence of SEQ ID NO: 116 (RefSeq NM_021956.4:294-3020 Homo sapiens glutamate ionotropic receptor kainate type subunit 2 (GRIK2), transcript variant 1, mRNA), as is shown in Table 4.

Additionally or alternatively, the Grik2 mRNA may be a polynucleotide having a nucleic acid sequence of SEQ ID NO: 117 or may be a variant thereof having at least 85% (e.g., at least 86%, 90%, 95%, 96%, 97%, 98%, 99%, or more) sequence identity to the nucleic acid sequence of SEQ ID NO: 117 (RefSeq NM_175768.3:294-2903 Homo sapiens glutamate ionotropic receptor kainate type subunit 2 (GRIK2), transcript variant 2, mRNA), as is shown in Table 4.

Additionally or alternatively, the Grik2 mRNA may be a polynucleotide having a nucleic acid sequence of SEQ ID NO: 118 or may be a variant thereof having at least 85% (e.g., at least 86%, 90%, 95%, 96%, 97%, 98%, 99%, or more) sequence identity to the nucleic acid sequence of SEQ ID NO: 118 (RefSeq NM_001166247.1:294-2972 Homo sapiens glutamate ionotropic receptor kainate type subunit 2 (GRIK2), transcript variant 3, mRNA), as is shown in Table 4.

Additionally or alternatively, the Grik2 mRNA may be a polynucleotide having a nucleic acid sequence of SEQ ID NO: 119 or may be a variant thereof having at least 85% (e.g., at least 86%, 90%, 95%, 96%, 97%, 98%, 99%, or more) sequence identity to the nucleic acid sequence of SEQ ID NO: 119 (RefSeq NM_001111268.2 Mus musculus glutamate ionotropic receptor kainate type subunit 2 (GRIK2), transcript variant 4, mRNA), as is shown below.

Additionally or alternatively, the Grik2 mRNA may be a polynucleotide having a nucleic acid sequence of SEQ ID NO: 120 or may be a variant thereof having at least 85% (e.g., at least 86%, 90%, 95%, 96%, 97%, 98%, 99%, or more) sequence identity to the nucleic acid sequence of SEQ ID NO: 120 (RefSeq NM_010349.4 Mus musculus glutamate ionotropic receptor kainate type subunit 2 (GRIK2), transcript variant 5, mRNA), as is shown in Table 4.

Additionally or alternatively, the Grik2 mRNA may be a polynucleotide having a nucleic acid sequence of SEQ ID NO: 121 or may be a variant thereof having at least 85% (e.g., at least 86%, 90%, 95%, 96%, 97%, 98%, 99%, or more) sequence identity to the nucleic acid sequence of SEQ ID NO: 121 (RefSeq NM_001358866 Mus musculus glutamate ionotropic receptor kainate type subunit 2 (GRIK2), transcript variant 6, mRNA), as is shown in Table 4.

Additionally or alternatively, the Grik2 mRNA may be a polynucleotide having a nucleic acid sequence of SEQ ID NO: 122 or may be a variant thereof having at least 85% (e.g., at least 86%, 90%, 95%, 96%, 97%, 98%, 99%, or more) sequence identity to the nucleic acid sequence of SEQ ID NO: 122 (RefSeq XM_015136995.2 Macaca mulatta glutamate ionotropic receptor kainate type subunit 2 (GRIK2), transcript variant 7, mRNA), as is shown in Table 4.

Additionally or alternatively, the Grik2 mRNA may be a polynucleotide having a nucleic acid sequence of SEQ ID NO: 123 or may be a variant thereof having at least 85% (e.g., at least 86%, 90%, 95%, 96%, 97%, 98%, 99%, or more) sequence identity to the nucleic acid sequence of SEQ ID NO: 123 (RefSeq XM_015136997.2 Macaca mulatta glutamate ionotropic receptor kainate type subunit 2 (GRIK2), transcript variant X1, mRNA), as is shown in Table 4.

Additionally or alternatively, the Grik2 mRNA may be a polynucleotide having a nucleic acid sequence of SEQ ID NO: 124 or may be a variant thereof having at least 85% (e.g., at least 86%, 90%, 95%, 96%, 97%, 98%, 99%, or more) sequence identity to the nucleic acid sequence of SEQ ID NO: 124 (RefSeq NM_019309.2 Rattus norvegicus glutamate ionotropic receptor kainate type subunit 2 (GRIK2), mRNA), as is shown in Table 4.

Additionally or alternatively, the Grik2 mRNA includes a polynucleotide corresponding to the mature GluK2 peptide coding sequence and having a nucleic acid sequence of SEQ ID NO: 125 or a variant thereof having at least 85% (e.g., at least 86%, 90%, 95%, 96%, 97%, 98%, 99%, or more) sequence identity to the nucleic acid sequence of SEQ ID NO: 125, as is shown in Table 4.

According to the disclosed methods and compositions, the Grik2 mRNA may include a 5′ UTR, such as, e.g., a 5′ UTR encoded by a polynucleotide having the nucleic acid sequence of SEQ ID NO: 126 or a variant thereof having at least 85% (e.g., at least 86%, 90%, 95%, 96%, 97%, 98%, 99%, or more) sequence identity to the nucleic acid sequence of SEQ ID NO: 126, as is shown in Table 4.

The Grik2 mRNA may also include a 3′ UTR, such as a 3′ UTR encoded by a polynucleotide having the nucleic acid sequence of SEQ ID NO: 127 or a variant thereof having at least 85% (e.g., at least 86%, 90%, 95%, 96%, 97%, 98%, 99%, or more) sequence identity to the nucleic acid sequence of SEQ ID NO: 127, as is shown in Table 4.

Additionally, the Grik2 mRNA may include a polynucleotide encoding the Grik2 signal peptide sequence, such as, e.g., a signal peptide sequence encoded by the nucleic acid sequence of SEQ ID NO: 128 or a variant thereof having at least 85% (e.g., at least 86%, 90%, 95%, 96%, 97%, 98%, 99%, or more) sequence identity to the nucleic acid sequence of SEQ ID NO: 128, as is shown in Table 4.

Grik2 mRNA Target Sequences

The ASO agents of the disclosure may target (e.g., specifically hybridize) to one or more regions of a Grik2 mRNA (e.g., one or more regions identified herein), such as, e.g., a translation initiation site (AUG codon), a sequence in the coding region (e.g. one or more of exons 1-16, which are described herein), or a region with the 5′ UTR or 3′ UTR of a Grik2 mRNA. By targeting these regions, the ASO agents of the disclosure can interfere with normal biological processing of the mRNA, including but not limited to translocation of the mRNA to the site for protein translation (e.g., translocation from the nucleus to the cytoplasm), translation of the mRNA into the GluK2 protein, splicing or maturation of the mRNA, and/or independent catalytic activity which may be engaged in by the RNA. The overall effect of such interference with the RNA function is to cause interference with Gluk2 protein expression, thereby reducing or eliminating GluK2 expression in the cell (e.g., neuron or astroglial cell).

Grik2 target sequences are portions or regions of the Grik2 mRNA sequence (e.g., the sense target sequence) that are amenable to inhibition or knockdown by antisense RNA. Several target sites of nucleic acids were identified as recognition sites of the targeted Grik2 transcript. Various antisense RNAs have been identified by the present inventors that hybridize to (or bind to) Grik2 target sites, as shown in Table 4 below. The Grik2 mRNA target nucleic acid includes a nucleotide sequence within regions of the primary transcript (RNA) or cDNA encoding the same. One skilled in the art would understand that the cDNA sequence is equivalent to the mRNA sequence, except for the substitution of uridines with thymidines, and can be used for the same purpose herein, i.e., the generation of an antisense oligonucleotide for inhibiting the expression if Grik2 mRNA.

Inhibitory RNA constructs (e.g., ASO agents disclosed herein) that may be used in conjunction with the methods and compositions disclosed herein include ASO agents capable of binding to (e.g., by complementary base pairing with) one or more (e.g., 1, 2, 3, 4, 5, 6, 7, 8, 9, 10, 12, 13, 14, 15, 16, 17, 18, 19, or more) target regions of a Grik2 mRNA, such as, e.g., within at least a portion of any one of the Grik2 mRNA transcripts of SEQ ID NOs: 115-125, 5′ UTR (SEQ ID NO: 126), 3′ UTR (SEQ ID NO: 127), nucleic acid sequence encoding a Grik2 signal peptide (SEQ ID NO: 128), exon 1 of Grik2 mRNA (SEQ ID NO: 129), exon 2 of Grik2 mRNA (SEQ ID NO: 130), exon 3 of Grik2 mRNA (SEQ ID NO: 131), exon 4 of Grik2 mRNA (SEQ ID NO: 132), exon of Grik2 mRNA (SEQ ID NO: 133), exon 6 of Grik2 mRNA (SEQ ID NO: 134), exon 7 of Grik2 mRNA (SEQ ID NO: 135), exon 8 of Grik2 mRNA (SEQ ID NO: 136), exon 9 of Grik2 mRNA (SEQ ID NO: 137), exon of Grik2 mRNA (SEQ ID NO: 138), exon 11 of Grik2 mRNA (SEQ ID NO: 139), exon 12 of Grik2 mRNA (SEQ ID NO: 140), exon 13 of Grik2 mRNA (SEQ ID NO: 141), exon 14 of Grik2 mRNA (SEQ ID NO: 142), exon of Grik2 mRNA (SEQ ID NO: 143), and exon 16 of Grik2 mRNA (SEQ ID NO: 144). The Grik2 ASO that targets a nucleic acid within at least a portion or region of SEQ ID NO: 115 or SEQ ID NO: 116 may be selected from an ASO agent listed in Table 2 or Table 3.

For example, the recombinant ASO agent of the disclosure includes a nucleotide sequence complementary to a nucleotide sequence within at least a portion or region of SEQ ID NO: 115. In another example, the ASO agent includes a nucleotide sequence complementary to a nucleotide sequence within at least a portion or region of SEQ ID NO: 116.

In a further example, the ASO agent of the disclosure that targets a Grik2 mRNA includes a nucleotide sequence complementary to a nucleotide sequence within at least a portion or region of the 5′ UTR (SEQ ID NO: 126). In another example, the ASO agent of the disclosure that targets a Grik2 mRNA includes a nucleotide sequence complementary to a nucleotide sequence within at least a portion or region of the 3′ UTR (SEQ ID NO: 127).

The disclosed ASO agents may hybridize to one or more exons of a Grik2 mRNA, such as, e.g., one or more exons of a Grik2 mRNA having a nucleic acid sequence of SEQ ID NO: 115 or a variant thereof having at least 85% (e.g., at least 86%, 90%, 95%, 96%, 97%, 98%, 99%, or more) sequence identity to the nucleic acid sequence of SEQ ID NO: 115. Accordingly, the ASO agent may hybridize within at least a portion or region of exon 1 of a Grik2 mRNA, such as, e.g., exon 1 of a Grik2 mRNA situated at nucleotide positions 1-408 of SEQ ID NO: 115. The sequence of exon 1 of the Grik2 mRNA may be a nucleic acid sequence of SEQ ID NO: 129 or a variant thereof having at least 85% (e.g., at least 86%, 90%, 95%, 96%, 97%, 98%, 99%, or more) sequence identity to the nucleic acid sequence of SEQ ID NO: 129, as is shown in Table 4.

For example, Grik2 target nucleic acids contemplated for targeting using the ASO agents disclosed herein include nucleotides 197-217 (SEQ ID NO: 115), 215-235 (SEQ ID NO: 115), 232-251 (SEQ ID NO: 115), 232-252 (SEQ ID NO: 115), 227-247 (SEQ ID NO: 115), 29-48 (SEQ ID NO: 116), 322-341 (SEQ ID NO: 115), 29-49 (SEQ ID NO: 116), 322-342 (SEQ ID NO: 115), 182-202 (SEQ ID NO: 115), 226-246 (SEQ ID NO: 115), 253-272 (SEQ ID NO: 115), 253-273 (SEQ ID NO: 115), 139-159 (SEQ ID NO: 115), 176-196 (SEQ ID NO: 115), 241-261 (SEQ ID NO: 115), 195-215 (SEQ ID NO: 115), 42-62 (SEQ ID NO: 115), 196-216 (SEQ ID NO: 115), or 30-49 (SEQ ID NO: 115). Additionally, the Grik2 ASO agents may hybridize to Grik2 mRNA within nucleotides 197-217 (SEQ ID NO: 115), 215-235 (SEQ ID NO: 115), 232-251 (SEQ ID NO: 115), 232-252 (SEQ ID NO: 115), 227-247 (SEQ ID NO: 115), 29-48 (SEQ ID NO: 116), 322-341 (SEQ ID NO: 115), 29-49 (SEQ ID NO: 116), 322-342 (SEQ ID NO: 115), 182-202 (SEQ ID NO: 115), 226-246 (SEQ ID NO: 115), 253-272 (SEQ ID NO: 115), 253-273 (SEQ ID NO: 115), 139-159 (SEQ ID NO: 115), 176-196 (SEQ ID NO: 115), 241-261 (SEQ ID NO: 115), 195-215 (SEQ ID NO: 115), 42-62 (SEQ ID NO: 115), 196-216 (SEQ ID NO: 115), 30-49 (SEQ ID NO: 115), or a fragment or portion thereof.

The Grik2 ASO agent that targets a nucleic acid within a portion or region of exon 1 of SEQ ID NO: 116 or SEQ ID NO: 115 may be selected from siRNA TJ (SEQ ID NO: 21), siRNA TG (SEQ ID NO: 23), siRNA TF (SEQ ID NO: 24), siRNA TE (SEQ ID NO: 25), siRNA TD (SEQ ID NO: 26), siRNA TC (SEQ ID NO: 28), siRNA CK (SEQ ID NO: 29), siRNA CX (SEQ ID NO: 42), siRNA CY (SEQ ID NO: 43), siRNA DO (SEQ ID NO: 45), siRNA D1 (SEQ ID NO: 46), siRNA D3 (SEQ ID NO: 48), siRNA XZ (SEQ ID NO: 54), siRNA YO (SEQ ID NO: 55), siRNA GF (SEQ ID NO: 64), siRNA ZZ (SEQ ID NO: 100), siRNA GE (SEQ ID NO: 65), siRNA GH (SEQ ID NO: 66), or siRNA YB (SEQ ID NO: 67), or an antisense oligonucleotide having at least than 85% (e.g., at least 86%, 90%, 95%, 96%, 97%, 98%, 99%, or more) sequence identity to any of siRNA TJ (SEQ ID NO: 21), siRNA TG (SEQ ID NO: 23), siRNA TF (SEQ ID NO: 24), siRNA TE (SEQ ID NO: 25), siRNA TD (SEQ ID NO: 26), siRNA TC (SEQ ID NO: 28), siRNA CK (SEQ ID NO: 29), siRNA CX (SEQ ID NO: 42), siRNA CY (SEQ ID NO: 43), siRNA DO (SEQ ID NO: 45), siRNA D1 (SEQ ID NO: 46), siRNA D3 (SEQ ID NO: 48), siRNA XZ (SEQ ID NO: 54), siRNA Y0 (SEQ ID NO: 55), siRNA GF (SEQ ID NO: 64), siRNA ZZ (SEQ ID NO: 100), siRNA GE (SEQ ID NO: 65), siRNA GH (SEQ ID NO: 66), or siRNA YB (SEQ ID NO: 67). The Grik2 ASO agent that targets a nucleic acid within a portion or region of exon 1 of SEQ ID NO: 116 or SEQ ID NO: 115 may exhibit at least 10% (e.g., at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 protein knockdown. Additionally, the Grik2 antisense oligonucleotide may be selected from siRNA TJ (SEQ ID NO: 21), siRNA TG (SEQ ID NO: 23), siRNA TF (SEQ ID NO: 24), siRNA TE (SEQ ID NO: 25), siRNA TD (SEQ ID NO: 26), siRNA TC (SEQ ID NO: 28), siRNA CK (SEQ ID NO: 29), siRNA CX (SEQ ID NO: 42), siRNA CY (SEQ ID NO: 43), siRNA DO (SEQ ID NO: 45), siRNA D1 (SEQ ID NO: 46), siRNA D3 (SEQ ID NO: 48), siRNA XZ (SEQ ID NO: 54), siRNA YO (SEQ ID NO: 55), siRNA GF (SEQ ID NO: 64), siRNA ZZ (SEQ ID NO: 100), siRNA GE (SEQ ID NO: 65), siRNA GH (SEQ ID NO: 66), or siRNA YB (SEQ ID NO: 67), or an antisense oligonucleotide having at least 85% (e.g., at least 86%, 90%, 95%, 96%, 97%, 98%, 99%, or more) sequence identity thereof, and exhibits at least 10% (e.g., at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

Additionally, the ASO agent may hybridize within at least a portion or region of exon 2 of a Grik2 mRNA, such as, e.g., exon 2 of the Grik2 mRNA situated at nucleotide positions 409-576 of SEQ ID NO: 115. The sequence of exon 2 of the Grik2 mRNA may be a nucleic acid sequence of SEQ ID NO: 130 or a variant thereof having at least 85% (e.g., at least 86%, 90%, 95%, 96%, 97%, 98%, 99%, or more) sequence identity to the nucleic acid sequence of SEQ ID NO: 130, as is shown in Table 4.

For example, Grik2 target nucleic acids contemplated for targeting using the ASO agents disclosed herein include nucleotides 501-521 of SEQ ID NO: 115 or nucleotides 208-228 of SEQ ID NO: 116, or a fragment or portion thereof.

The Grik2 ASO agent that targets a nucleic acid within a portion or region of exon 2 of SEQ ID NO: 116 or SEQ ID NO: 115 is siRNA GO (SEQ ID NO: 1), or an antisense oligonucleotide having greater than 85% (e.g., at least 86%, 90%, 95%, 96%, 97%, 98%, 99%, or more) sequence identity to siRNA GO (SEQ ID NO: 1). In other embodiments, the Grik2 antisense oligonucleotide that targets a nucleic acid within a portion or region of exon 2 of SEQ ID NO: 116 or SEQ ID NO: 115 exhibits greater than 75% GluK2 knockdown. In still other embodiments, the Grik2 antisense oligonucleotide is siRNA GO (SEQ ID NO: 1), or an antisense oligonucleotide having greater than 85% (e.g., at least 86%, 90%, 95%, 96%, 97%, 98%, 99%, or more) sequence identity thereof, and exhibits greater than 75% (e.g., at least 75%, 80%, 85%, 90%, 95%, 96%, 97%, 98%, 99%, or more) GluK2 knockdown.

The ASO agent may also hybridize within at least a portion or region of exon 3 of a Grik2 mRNA, such as, e.g., exon 3 of the Grik2 mRNA situated at nucleotide positions 577-834 of SEQ ID NO: 115. The sequence of exon 3 of the Grik2 mRNA may be a nucleic acid sequence of SEQ ID NO: 131 or a variant thereof having at least 85% (e.g., at least 86%, 90%, 95%, 96%, 97%, 98%, 99%, or more) sequence identity to the nucleic acid sequence of SEQ ID NO: 131, as is shown in Table 4.

For example, Grik2 target nucleic acids contemplated for targeting using the ASO agents disclosed herein include nucleotides 307-327 of SEQ ID NO: 116 or nucleotides 600-620 SEQ ID NO: 115, nucleotides 352-372 of SEQ ID NO: 116 or nucleotides 645-665 of SEQ ID NO: 115, nucleotides 381-400 of SEQ ID NO: 116 or nucleotides 674-693 of SEQ ID NO: 115, nucleotides 381-401 of SEQ ID NO: 116 or nucleotides 674-694 of SEQ ID NO: 115, nucleotides 380-400 of SEQ ID NO: 116 or nucleotides 673-693 of SEQ ID NO: 115, nucleotides 534-554 of SEQ ID NO: 116 or nucleotides 827-847 of SEQ ID NO: 115, nucleotides 308-328 of SEQ ID NO: 116 or nucleotides 601-621 of SEQ ID NO: 115, nucleotides 396-416 of SEQ ID NO: 116 or nucleotides 689-709 of SEQ ID NO: 115, nucleotides 355-375 of SEQ ID NO: 116 or nucleotides 648-668 of SEQ ID NO: 115, nucleotides 357-377 of SEQ ID NO: 116 or nucleotides 650-670 of SEQ ID NO: 115, nucleotides 424-444 of SEQ ID NO: 116 or nucleotides 717-737 of SEQ ID NO: 115, nucleotides 429-449 of SEQ ID NO: 116 or nucleotides 722-742 SEQ ID NO: 115, or a fragment or portion thereof.

The Grik2 ASO agent that targets a nucleic acid within a portion or region of exon 3 of SEQ ID NO: 116 or SEQ ID NO: 115 is selected from siRNA TV (SEQ ID NO: 2), siRNA TU (SEQ ID NO: 3), siRNA CL (SEQ ID NO: 30), siRNA CM (SEQ ID NO: 31), siRNA CR (SEQ ID NO: 36), siRNA CV (SEQ ID NO: 40), siRNA Y4 (SEQ ID NO: 59), siRNA MP (SEQ ID NO: 76), siRNA MW (SEQ ID NO: 80), siRNA MV (SEQ ID NO: 81), siRNA G8 (SEQ ID NO: 92), or siRNA MF (SEQ ID NO: 93), or an ASO having greater than 85% (e.g., at least 86%, 90%, 95%, 96%, 97%, 98%, 99%, or more) sequence identity to siRNA TV (SEQ ID NO: 2), siRNA TU (SEQ ID NO: 3), siRNA CL (SEQ ID NO: 30), siRNA CM (SEQ ID NO: 31), siRNA CR (SEQ ID NO: 36), siRNA CV (SEQ ID NO: 40), siRNA Y4 (SEQ ID NO: 59), siRNA MP (SEQ ID NO: 76), siRNA MW (SEQ ID NO: 80), siRNA MV (SEQ ID NO: 81), siRNA G8 (SEQ ID NO: 92), or siRNA MF (SEQ ID NO: 93). Additionally, the Grik2 ASO that targets a nucleic acid within a portion or region of exon 3 of SEQ ID NO: 116 or SEQ ID NO: 115 may exhibit at least 15% (e.g., at least 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown. In still other embodiments, the Grik2 ASO is selected from siRNA TV (SEQ ID NO: 2), siRNA TU (SEQ ID NO: 3), siRNA CL (SEQ ID NO: 30), siRNA CM (SEQ ID NO: 31), siRNA CR (SEQ ID NO: 36), siRNA CV (SEQ ID NO: 40), siRNA Y4 (SEQ ID NO: 59), siRNA MP (SEQ ID NO: 76), siRNA MW (SEQ ID NO: 80), siRNA MV (SEQ ID NO: 81), siRNA G8 (SEQ ID NO: 92), or siRNA MF (SEQ ID NO: 93), or an ASO having greater than 85% (e.g., at least 86%, 90%, 95%, 96%, 97%, 98%, 99%, or more) sequence identity thereof, and exhibits greater than 15% (e.g., at least 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

Furthermore, the disclosed ASO agent may hybridize within at least a portion or region of exon 4 of a Grik2 mRNA, such as, e.g., exon 4 of the Grik2 mRNA situated at nucleotide positions 835-1016 of SEQ ID NO: 115. The sequence of exon 4 of the Grik2 mRNA may be a nucleic acid sequence of SEQ ID NO: 132 or a variant thereof having at least 85% (e.g., at least 86%, 90%, 95%, 96%, 97%, 98%, 99%, or more) sequence identity to the nucleic acid sequence of SEQ ID NO: 132, as is shown in Table 4.

For example, Grik2 target nucleic acids contemplated for targeting using the ASO agents disclosed herein include nucleotides 534-554 of SEQ ID NO: 116 or nucleotides 827-847 of SEQ ID NO: 115, nucleotides 579-599 of SEQ ID NO: 116) or nucleotides 872-892 of SEQ ID NO: 115, nucleotides 717-737 of SEQ ID NO: 116 or nucleotides 1010-1030 of SEQ ID NO: 115, nucleotides 721-741 of SEQ ID NO: 116 or nucleotides 1014-1034 of SEQ ID NO: 115), and nucleotides 559-579 of SEQ ID NO: 116 or nucleotides 852-872 of SEQ ID NO: 115, or a fragment or a portion thereof.

The Grik2 ASO that targets a nucleic acid within a portion or region of exon 4 of SEQ ID NO: 116 or SEQ ID NO: 115 is selected from siRNA CV (SEQ ID NO: 40), siRNA Y(SEQ ID NO: 60), siRNA G9 (SEQ ID NO: 68), siRNA MD (SEQ ID NO: 70), or siRNA MK (SEQ ID NO: 86), or an ASO having greater than 85% (e.g., at least 86%, 90%, 95%, 96%, 97%, 98%, 99%, or more) sequence identity to siRNA CV (SEQ ID NO: 40), siRNA Y(SEQ ID NO: 60), siRNA G9 (SEQ ID NO: 68), siRNA MD (SEQ ID NO: 70), or siRNA MK (SEQ ID NO: 86). Additionally, the Grik2 ASO that targets a nucleic acid within a portion or region of exon 4 of SEQ ID NO: 116 or SEQ ID NO: 115 exhibits greater than 25% (e.g., at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is selected from siRNA CV (SEQ ID NO: 40), siRNA Y(SEQ ID NO: 60), siRNA G9 (SEQ ID NO: 68), siRNA MD (SEQ ID NO: 70), or siRNA MK (SEQ ID NO: 86), or an ASO having greater than 85% (e.g., at least 86%, 90%, 95%, 96%, 97%, 98%, 99%, or more) sequence identity thereof, and exhibits greater than 25% (e.g., at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

Additionally, the ASO agent may hybridize within at least a portion or region of exon of a Grik2 mRNA, such as, e.g., exon of the Grik2 mRNA situated at nucleotide positions 1017-1070 of SEQ ID NO: 115. The sequence of exon of the Grik2 mRNA may be a nucleic acid sequence of SEQ ID NO: 133 or a variant thereof having at least 85% (e.g., at least 86%, 90%, 95%, 96%, 97%, 98%, 99%, or more) sequence identity to the nucleic acid sequence of SEQ ID NO: 133, as is shown in Table 4.

For example, Grik2 target nucleic acids contemplated for targeting using the ASO agents disclosed herein include nucleotides 717-737 of SEQ ID NO: 116 or nucleotides 1010-1030 of SEQ ID NO: 115, nucleotides 728-747 of SEQ ID NO: 116 or nucleotides 1021-1040 of SEQ ID NO: 115, and nucleotides 721-741 of SEQ ID NO: 116 or nucleotides 1014-1034 of SEQ ID NO: 115, or a fragment or portion thereof.

The Grik2 ASO that targets a nucleic acid within a portion or region of exon of SEQ ID NO: 116 or SEQ ID NO: 115 is selected from siRNA G9 (SEQ ID NO: 68), siRNA ME (SEQ ID NO: 69), or siRNA MD (SEQ ID NO: 70), or an ASO having greater than 85% (e.g., at least 86%, 90%, 95%, 96%, 97%, 98%, 99%, or more) sequence identity to siRNA G9 (SEQ ID NO: 68), siRNA ME (SEQ ID NO: 69)SEQ ID NO: 69), or siRNA MD (SEQ ID NO: 70). Additionally, the Grik2 ASO that targets a nucleic acid within a portion or region of exon of SEQ ID NO: 116 or SEQ ID NO: 115 exhibits greater than 75% (e.g., at least 75%, 80%, 85%, 90%, 95%, 96%, 97%, 98%, 99%, or more) GluK2 knockdown. Further still, the Grik2 ASO is selected from siRNA G9 (SEQ ID NO: 68), siRNA ME (SEQ ID NO: 69), or siRNA MD (SEQ ID NO: 70), or an ASO having greater than 85% (e.g., at least 86%, 90%, 95%, 96%, 97%, 98%, 99%, or more) sequence identity thereof, and exhibits greater than 75% (e.g., at least 75%, 80%, 85%, 90%, 95%, 96%, 97%, 98%, 99%, or more) GluK2 knockdown.

The ASO agent may also hybridize within at least a portion or region of exon 6 of a Grik2 mRNA, such as, e.g., exon 6 of the Grik2 mRNA situated at nucleotide positions 1071-1244 of SEQ ID NO: 115. The sequence of exon 6 of the Grik2 mRNA may be a nucleic acid sequence of SEQ ID NO: 134 or a variant thereof having at least 85% (e.g., at least 86%, 90%, 95%, 96%, 97%, 98%, 99%, or more) sequence identity to the nucleic acid sequence of SEQ ID NO: 134, as is shown in Table 4.

For example, Grik2 target nucleic acids contemplated for targeting using the ASO agents disclosed herein include nucleotides 806-826 of SEQ ID NO: 116 or nucleotides 1099-1119 of SEQ ID NO: 115, nucleotides 905-925 of SEQ ID NO: 116 or nucleotides 1198-1218 of SEQ ID NO: 115, nucleotides 904-924 of SEQ ID NO: 116 or nucleotides 1197-1217 of SEQ ID NO: 115, nucleotides 885-905 of SEQ ID NO: 116 or nucleotides 1178-1198 of SEQ ID NO: 115, nucleotides 908-927 of SEQ ID NO: 116 or nucleotides 1201-1220 of SEQ ID NO: 115, nucleotides 908-928 of SEQ ID NO: 116 or nucleotides 1201-1221 of SEQ ID NO: 115, nucleotides 934-954 of SEQ ID NO: 116 or nucleotides 1227-15 1247 of SEQ ID NO: 115, nucleotides 931-950 of SEQ ID NO: 116 or nucleotides 1224-1243 of SEQ ID NO: 115, and nucleotides 938-957 of SEQ ID NO: 116 or nucleotides 1231-1250 of SEQ ID NO: 115, or a fragment or portion thereof.

The Grik2 ASO that targets a nucleic acid within a portion or region of exon 6 of SEQ ID NO: 116 or SEQ ID NO: 115 is selected from siRNA TT (SEQ ID NO: 4), siRNA G1 (SEQ ID NO: 5), siRNA G2 (SEQ ID NO: 6), siRNA Y1 (SEQ ID NO: 56), siRNA Y2 (SEQ ID NO: 57), siRNA Y3 (SEQ ID NO: 58), siRNA GG (SEQ ID NO: 91), siRNA MH (SEQ ID NO: 94), or siRNA MG (SEQ ID NO: 95), or an ASO having greater than 85% (e.g., at least 86%, 90%, 95%, 96%, 97%, 98%, 99%, or more) sequence identity to siRNA TT (SEQ ID NO: 4), siRNA G1 (SEQ ID NO: 5), siRNA G2 (SEQ ID NO: 6), siRNA Y1 (SEQ ID NO: 56), siRNA Y2 (SEQ ID NO: 57), siRNA Y3 (SEQ ID NO: 58), siRNA GG (SEQ ID NO: 91), siRNA MH (SEQ ID NO: 94), or siRNA MG (SEQ ID NO: 95). Additionally, the Grik2 ASO that targets a nucleic acid within a portion or region of exon 6 of SEQ ID NO: 116 or SEQ ID NO: 115 exhibits greater than 20% (e.g., at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown. In still other embodiments, the Grik2 ASO is selected from siRNA TT (SEQ ID NO: 4), siRNA G1 (SEQ ID NO: 5), siRNA G2 (SEQ ID NO: 6), siRNA Y1 (SEQ ID NO: 56), siRNA Y2 (SEQ ID NO: 57), siRNA Y3 (SEQ ID NO: 58), siRNA GG (SEQ ID NO: 91), siRNA MH (SEQ ID NO: 94), or siRNA MG (SEQ ID NO: 95), or an ASO having greater than 85% (e.g., at least 86%, 90%, 95%, 96%, 97%, 98%, 99%, or more) sequence identity thereof, and exhibits greater than 20% (e.g., at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

In addition, the ASO agent may hybridize within at least a portion or region of exon 7 of a Grik2 mRNA, such as, e.g., exon 7 of the Grik2 mRNA situated at nucleotide positions 1245-1388 of SEQ ID NO: 115. The sequence of exon 7 of the Grik2 mRNA may be a nucleic acid sequence of SEQ ID NO: 135 or a variant thereof having at least 85% (e.g., at least 86%, 90%, 95%, 96%, 97%, 98%, 99%, or more) sequence identity to the nucleic acid sequence of SEQ ID NO: 135, as is shown in Table 4.

For example, Grik2 target nucleic acids contemplated for targeting using the ASO agents disclosed herein include nucleotides 1029-1049 of SEQ ID NO: 116 or nucleotides 1322-1342 of SEQ ID NO: 115, nucleotides 985-1005 of SEQ ID NO: 116 or nucleotides 1278-1298 of SEQ ID NO: 115, nucleotides 1057-1077 of SEQ ID NO: 116 or nucleotides 1350-1370 of SEQ ID NO: 115, nucleotides 1058-1078 of SEQ ID NO: 116 or nucleotides 1351-1371 of SEQ ID NO: 115, and nucleotides 1043-1063 of SEQ ID NO: 116 or nucleotides 1336-1356 of SEQ ID NO: 115, or a fragment or portion thereof.

The Grik2 ASO that targets a nucleic acid within a portion or region of exon 7 of SEQ ID NO: 116 or SEQ ID NO: 115 is selected from siRNA TL (SEQ ID NO: 20), siRNA CS (SEQ ID NO: 37), siRNA CT (SEQ ID NO: 38), siRNA CZ (SEQ ID NO: 44), or siRNA D2 (SEQ ID NO: 47), or an ASO having greater than 85% (e.g., at least 86%, 90%, 95%, 96%, 97%, 98%, 99%, or more) sequence identity to siRNA TL (SEQ ID NO: 20), siRNA CS (SEQ ID NO: 37), siRNA CT (SEQ ID NO: 38), siRNA CZ (SEQ ID NO: 44), or siRNA D2 (SEQ ID NO: 47). Additionally, the Grik2 ASO that targets a nucleic acid within a portion or region of exon 7 of SEQ ID NO: 116 or SEQ ID NO: 115 exhibits greater than 45% (e.g., at least 45%, 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is selected from siRNA TL (SEQ ID NO: 20), siRNA CS (SEQ ID NO: 37), siRNA CT (SEQ ID NO: 38), siRNA CZ (SEQ ID NO: 44), or siRNA D2 (SEQ ID NO: 47), or an ASO having greater than 85% (e.g., at least 86%, 90%, 95%, 96%, 97%, 98%, 99%, or more) sequence identity thereof, and exhibits greater than 45% (e.g., at least 45%, 50%, 55%, 60%, 65%, 70%, 75%, 80%, 85%, 90%, 91%, 92%, 93%, 94%, 95%, 96%, 97%, 98%, 99% or more) GluK2 knockdown.

The ASO agent may further hybridize within at least a portion or region of exon 8 of a Grik2 mRNA, such as, e.g., exon 8 of the Grik2 mRNA situated at nucleotide positions 1389-1496 of SEQ ID NO: 115. The sequence exon 8 of the Grik2 mRNA may be a nucleic acid sequence of SEQ ID NO: 136 or a variant thereof having at least 85% (e.g., at least 86%, 90%, 95%, 96%, 97%, 98%, 99%, or more) sequence identity to the nucleic acid sequence of SEQ ID NO: 136, as is shown in Table 4. The ASO agent that targets a portion or a region of exon 8 may exhibit at least 10% (e.g., at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

Furthermore, the ASO agent may hybridize within at least a portion or region of exon 9 of a Grik2 mRNA, such as, e.g., exon 9 of the Grik2 mRNA situated at nucleotide positions 1497-1610 of SEQ ID NO: 115. The sequence of exon 9 of the Grik2 mRNA may be a nucleic acid sequence of SEQ ID NO: 137 or a variant thereof having at least 85% (e.g., at least 86%, 90%, 95%, 96%, 97%, 98%, 99%, or more) sequence identity to the nucleic acid sequence of SEQ ID NO: 137, as is shown in Table 4.

For example, Grik2 target nucleic acids contemplated for targeting using the ASO agents disclosed herein include nucleotides 1252-1272 of SEQ ID NO: 116 or nucleotides 1545-1565 of SEQ ID NO: 115, or a fragment or portion thereof.

The Grik2 ASO that targets a nucleic acid within a portion or region of exon 9 of SEQ ID NO: 116 or SEQ ID NO: 115 is siRNA TQ (SEQ ID NO: 12), or an ASO having greater than 85% (e.g., at least 86%, 90%, 95%, 96%, 97%, 98%, 99%, or more) sequence identity to siRNA TQ (SEQ ID NO: 12). Additionally, the Grik2 ASO that targets a nucleic acid within a portion or region of exon 9 of SEQ ID NO: 116 or SEQ ID NO: 115 exhibits greater than 50% (e.g., at least 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is siRNA TQ (SEQ ID NO: 12), or an ASO having greater than 85% (e.g., at least 86%, 90%, 95%, 96%, 97%, 98%, 99%, or more) sequence identity thereof, and exhibits greater than 50% (e.g., at least 50%, 55%, 60%, 65%, 70%, 75%, 80%, 85%, 90%, 91%, 92%, 93%, 94%, 95%, 96%, 97%, 98%, 99% or more) GluK2 knockdown.

The ASO agent may additionally hybridizes to exon of a Grik2 mRNA, such as, e.g., exon of the Grik2 mRNA situated at nucleotide positions 1611-1817 of SEQ ID NO: 115. The sequence of exon of the Grik2 mRNA may be a nucleic acid sequence of SEQ ID NO: 138 or a variant thereof having at least 85% (e.g., at least 86%, 90%, 95%, 96%, 97%, 98%, 99%, or more) sequence identity to the nucleic acid sequence of SEQ ID NO: 138, as is shown in Table 4.

For example, Grik2 target nucleic acids contemplated for targeting using the ASO agents disclosed herein include nucleotides 1396-1416 of SEQ ID NO: 116 or nucleotides 1689-1709 of SEQ ID NO: 115, nucleotides 1496-1516 of SEQ ID NO: 116 or nucleotides 1789-1809 of SEQ ID NO: 115, nucleotides 1417-1437 of SEQ ID NO: 116 or nucleotides 1710-1730 of SEQ ID NO: 115, nucleotides 1483-1503 of SEQ ID NO: 116 or nucleotides 1776-1796 of SEQ ID NO: 115, and nucleotides 1491-1511 of SEQ ID NO: 116 or nucleotides 1784-1804 of SEQ ID NO: 115, or a fragment or portion thereof.

The Grik2 ASO that targets a nucleic acid within a portion or region of exon of SEQ ID NO: 116 or SEQ ID NO: 115 is selected from siRNA GD (SEQ ID NO: 7), G3 (SEQ ID NO: 8), siRNA MU (SEQ ID NO: 96), siRNA MT (SEQ ID NO: 98), or siRNA MS (SEQ ID NO: 99), or an ASO having greater than 85% (e.g., at least 86%, 90%, 95%, 96%, 97%, 98%, 99%, or more) sequence identity to siRNA GD (SEQ ID NO: 7), G3 (SEQ ID NO: 8), siRNA MU (SEQ ID NO: 96), siRNA MT (SEQ ID NO: 98), or siRNA MS (SEQ ID NO: 99). Additionally, the Grik2 ASO that targets a nucleic acid within a portion or region of exon of SEQ ID NO: 116 or SEQ ID NO: 115 exhibits greater than 50% (e.g., at least 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is selected from GD (SEQ ID NO: 7), G3 (SEQ ID NO: 8), siRNA MU (SEQ ID NO: 96), siRNA MT (SEQ ID NO: 98), or siRNA MS (SEQ ID NO: 99), or an ASO having greater than 85% (e.g., at least 86%, 90%, 95%, 96%, 97%, 98%, 99%, or more) sequence identity thereof, and exhibits greater than 50% (e.g., at least 50%, 55%, 60%, 65%, 70%, 75%, 80%, 85%, 90%, 91%, 92%, 93%, 94%, 95%, 96%, 97%, 98%, 99% or more) GluK2 knockdown.

Furthermore, the ASO agent may hybridize within at least a portion or region of exon 11 of a Grik2 mRNA, such as, e.g., exon 11 of the Grik2 mRNA situated at nucleotide positions 1818-2041 of SEQ ID NO: 115. The sequence of exon 11 of the Grik2 mRNA may be a nucleic acid sequence of SEQ ID NO: 139 or a variant thereof having at least 85% (e.g., at least 86%, 90%, 95%, 96%, 97%, 98%, 99%, or more) sequence identity to the nucleic acid sequence of SEQ ID NO: 139, as is shown in Table 4.

For example, Grik2 target nucleic acids contemplated for targeting using the ASO agents disclosed herein include nucleotides 1550-1570 of SEQ ID NO: 116 or nucleotides 1843-1863 of SEQ ID NO: 115, nucleotides 1637-1657 of SEQ ID NO: 116 or nucleotides 1930-1950 of SEQ ID NO: 115, nucleotides 1670-1690 of SEQ ID NO: 116 or nucleotides 1963-1983 of SEQ ID NO: 115, nucleotides 1565-1585 of SEQ ID NO: 116 or nucleotides 1858-1878 of SEQ ID NO: 115, nucleotides 1550-1569 of SEQ ID NO: 116 or nucleotides 1843-1862 of SEQ ID NO: 115, nucleotides 1544-1563 of SEQ ID NO: 116 or nucleotides 1837-1856 of SEQ ID NO: 115, nucleotides 1544-1564 of SEQ ID NO: 116 or nucleotides 1837-1857 of SEQ ID NO: 115, nucleotides 1526-1546 of SEQ ID NO: 116, and nucleotides 1541-1561 of SEQ ID NO: 116 or nucleotides 1834-1854 of SEQ ID NO: 115, or a fragment or portion thereof.

The Grik2 ASO that targets a nucleic acid within a portion or region of exon 11 of SEQ ID NO: 116 or SEQ ID NO: 115 is selected from siRNA TH (SEQ ID NO: 22), siRNA CU (SEQ ID NO: 39), siRNA Y7 (SEQ ID NO: 62), siRNA TK (SEQ ID NO: 74), siRNA TI (SEQ ID NO: 75), siRNA Y8 (SEQ ID NO: 87), siRNA Y9 (SEQ ID NO: 88), siRNA MJ (SEQ ID NO: 89), or siRNA MI (SEQ ID NO: 90), or an ASO having greater than 85% (e.g., at least 86%, 90%, 95%, 96%, 97%, 98%, 99%, or more) sequence identity siRNA TH (SEQ ID NO: 22), siRNA CU (SEQ ID NO: 39), siRNA Y7 (SEQ ID NO: 62), siRNA TK (SEQ ID NO: 74), siRNA TI (SEQ ID NO: 75), siRNA Y8 (SEQ ID NO: 87), siRNA Y9 (SEQ ID NO: 88), siRNA MJ (SEQ ID NO: 89), or siRNA MI (SEQ ID NO: 90). Additionally, the Grik2 ASO that targets a nucleic acid within a portion or region of exon 11 of SEQ ID NO: 116 or SEQ ID NO: 115 exhibits greater than 25% (e.g., at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is selected from siRNA TH (SEQ ID NO: 22), siRNA CU (SEQ ID NO: 39), siRNA Y7 (SEQ ID NO: 62), siRNA TK (SEQ ID NO: 74), siRNA TI (SEQ ID NO: 75), siRNA Y8 (SEQ ID NO: 87), siRNA Y9 (SEQ ID NO: 88), siRNA MJ (SEQ ID NO: 89), or siRNA MI (SEQ ID NO: 90), or an ASO having greater than 85% (e.g., at least 86%, 90%, 95%, 96%, 97%, 98%, 99%, or more) sequence identity thereof, and exhibits greater than 25% (e.g., at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

As a further example, the ASO agent may hybridize within at least a portion or region of exon 12 of a Grik2 mRNA, such as e.g., exon 12 of the Grik2 mRNA situated at nucleotide positions 2042-2160 of SEQ ID NO: 115. The sequence of exon 12 of the Grik2 mRNA may be a nucleic acid sequence of SEQ ID NO: 140 or a variant thereof having at least 85% (e.g., at least 86%, 90%, 95%, 96%, 97%, 98%, 99%, or more) sequence identity to the nucleic acid sequence of SEQ ID NO: 140, as is shown in Table 4.

For example, Grik2 target nucleic acids contemplated for targeting using the ASO agents disclosed herein include nucleotides 1786-1805 of SEQ ID NO: 116 or nucleotides 2079-2098 of SEQ ID NO: 115, nucleotides 1786-1806 of SEQ ID NO: 116 or nucleotides 2079-2099 of SEQ ID NO: 115, nucleotides 1778-1797 of SEQ ID NO: 116 or nucleotides 2071-2090 of SEQ ID NO: 115, and nucleotides 1836-1856 of SEQ ID NO: 116 or nucleotides 2129-2149 of SEQ ID NO: 115, or a fragment or portion thereof.

The Grik2 ASO that targets a nucleic acid within a portion or region of exon 12 of SEQ ID NO: 116 or SEQ ID NO: 115 selected from siRNA XX (SEQ ID NO: 82), siRNA XY (SEQ ID NO: 83), siRNA MM (SEQ ID NO: 84), or siRNA ML (SEQ ID NO: 85), or an ASO having greater than 85% (e.g., at least 86%, 90%, 95%, 96%, 97%, 98%, 99%, or more) sequence identity to siRNA XX (SEQ ID NO: 82), siRNA XY (SEQ ID NO: 83), siRNA MM (SEQ ID NO: 84), or siRNA ML (SEQ ID NO: 85). Additionally, the Grik2 ASO that targets a nucleic acid within a portion or region of exon 12 of SEQ ID NO: 116 or SEQ ID NO: 115 exhibits greater than 50% (e.g., at least 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is selected from siRNA XX (SEQ ID NO: 82), siRNA XY (SEQ ID NO: 83), siRNA MM (SEQ ID NO: 84), or siRNA ML (SEQ ID NO: 85), or an ASO having greater than 85% (e.g., at least 86%, 90%, 95%, 96%, 97%, 98%, 99%, or more) sequence identity thereof, and exhibits greater than 50% (e.g., at least 50%, 55%, 60%, 65%, 70%, 75%, 80%, 85%, 90%, 91%, 92%, 93%, 94%, 95%, 96%, 97%, 98%, 99% or more) GluK2 knockdown.

The ASO agent may also hybridize within at least a portion or region of exon 13 of a Grik2 mRNA, such as, e.g., exon 13 of the Grik2 mRNA situated at nucleotide positions 2161-2378 of SEQ ID NO: 115. The sequence of exon 13 of the Grik2 mRNA may be a nucleic acid sequence of SEQ ID NO: 141 or a variant thereof having at least 85% (e.g., at least 86%, 90%, 95%, 96%, 97%, 98%, 99%, or more) sequence identity to the nucleic acid sequence of SEQ ID NO: 141, as is shown in Table 4.

For example, Grik2 target nucleic acids contemplated for targeting using the ASO agents disclosed herein include nucleotides 1968-1987 of SEQ ID NO: 116 or nucleotides 2213-2233 of SEQ ID NO: 115, nucleotides 1968-1988 of SEQ ID NO: 116 or nucleotides 2213-2233 of SEQ ID NO: 115, nucleotides 1906-1926 of SEQ ID NO: 116 or nucleotides 2199-2219 of SEQ ID NO: 115, and nucleotides 1920-1940 of SEQ ID NO: 116 or nucleotides 2213-2233 of SEQ ID NO: 115, or a fragment or portion thereof.

The Grik2 ASO that targets a nucleic acid within a portion or region of exon 13 of SEQ ID NO: 116 or SEQ ID NO: 115 is selected from siRNA TP (SEQ ID NO: 13), siRNA TO (SEQ ID NO: 14), siRNA MR (SEQ ID NO: 72), or siRNA MQ (SEQ ID NO: 73), or an ASO having greater than 85% (e.g., at least 86%, 90%, 95%, 96%, 97%, 98%, 99%, or more) sequence identity to siRNA TP (SEQ ID NO: 13), siRNA TO (SEQ ID NO: 14), siRNA MR (SEQ ID NO: 72), or siRNA MQ (SEQ ID NO: 73). Additionally, the Grik2 ASO that targets a nucleic acid within a portion or region of exon 13 of SEQ ID NO: 116 or SEQ ID NO: 115 exhibits greater than 35% (e.g., at least 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is selected from siRNA TP (SEQ ID NO: 13), siRNA TO (SEQ ID NO: 14), siRNA MR (SEQ ID NO: 72), or siRNA MQ (SEQ ID NO: 73), or an ASO having greater than 85% (e.g., at least 86%, 90%, 95%, 96%, 97%, 98%, 99%, or more) sequence identity thereof, and exhibits greater than 35% (e.g., at least 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

Additionally, the ASO agent may hybridize within at least a portion or region of exon 14 of a Grik2 mRNA, such as, e.g., exon 14 of the Grik2 mRNA situated at nucleotide positions 2379-2604 of SEQ ID NO: 115. The sequence of exon 14 of the Grik2 mRNA may be a nucleic acid sequence of SEQ ID NO: 142 or a variant thereof having at least 85% (e.g., at least 86%, 90%, 95%, 96%, 97%, 98%, 99%, or more) sequence identity to the nucleic acid sequence of SEQ ID NO: 142, as is shown in Table 4. 30 For example, Grik2 target nucleic acids contemplated for targeting using the ASO agents disclosed herein include nucleotides 2209-2228 of SEQ ID NO: 116 or nucleotides 2502-2521 of SEQ ID NO: 115, nucleotides 2209-2229 of SEQ ID NO: 116 or nucleotides 2502-2522 of SEQ ID NO: 115, nucleotides 2308-2328 of SEQ ID NO: 116 or nucleotides 2601-2621 of SEQ ID NO: 115, nucleotides 2304-2323 of SEQ ID NO: 116) or nucleotides 2597-2616 of SEQ ID NO: 115, and nucleotides 2303-2323 of SEQ ID NO: 116 or nucleotides 2596-2616 of SEQ ID NO: 115, or a fragment or portion thereof.

The Grik2 ASO that targets a nucleic acid within a portion or region of exon 14 of SEQ ID NO: 116 or SEQ ID NO: 115 is selected from siRNA CP (SEQ ID NO: 34), siRNA CQ (SEQ ID NO: 35), siRNA GI (SEQ ID NO: 77), siRNA MO (SEQ ID NO: 78), or siRNA MN (SEQ ID NO: 79), or an ASO having greater than 85% (e.g., at least 86%, 90%, 95%, 96%, 97%, 98%, 99%, or more) sequence identity to siRNA CP (SEQ ID NO: 34), siRNA CQ (SEQ ID NO: 35), siRNA GI (SEQ ID NO: 77), siRNA MO (SEQ ID NO: 78), or siRNA MN (SEQ ID NO: 79). Additionally, the Grik2 ASO that targets a nucleic acid within a portion or region of exon 14 of SEQ ID NO: 116 or SEQ ID NO: 115 exhibits greater than 35% (e.g., at least 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is selected from siRNA CP (SEQ ID NO: 34), siRNA CQ (SEQ ID NO: 35), siRNA GI (SEQ ID NO: 77), siRNA MO (SEQ ID NO: 78), or siRNA MN (SEQ ID NO: 79), or an ASO having greater than 85% (e.g., at least 86%, 90%, 95%, 96%, 97%, 98%, 99%, or more) sequence identity thereof, and exhibits greater than 35% (e.g., at least 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

The ASO agent may also hybridize within at least a portion or region of exon of a Grik2 mRNA, such as, e.g., exon of the Grik2 mRNA situated at nucleotide positions 2605-2855 of SEQ ID NO: 115. The nucleotide sequence of exon of the Grik2 mRNA may be a nucleic acid sequence of SEQ ID NO: 143 or a variant thereof having at least 85% (e.g., at least 86%, 90%, 95%, 96%, 97%, 98%, 99%, or more) sequence identity to the nucleic acid sequence of SEQ ID NO: 143, as is shown in Table 4.

For example, Grik2 target nucleic acids contemplated for targeting using the ASO agents disclosed herein include nucleotides 2309-2329 of SEQ ID NO: 116 or nucleotides 2602-2622 of SEQ ID NO: 115, or a fragment or portion thereof.

The Grik2 ASO that targets a nucleic acid within a portion or region of exon of SEQ ID NO: 116 or SEQ ID NO: 115 is siRNA XU (SEQ ID NO: 51), or an ASO having greater than 85% (e.g., at least 86%, 90%, 95%, 96%, 97%, 98%, 99%, or more) sequence identity to siRNA XU (SEQ ID NO:X). Additionally, the Grik2 ASO that targets a nucleic acid within a portion or region of exon of SEQ ID NO: 116 or SEQ ID NO: 115 exhibits greater than 50% (e.g., at least 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is siRNA XU (SEQ ID NO: 51), or an ASO having greater than 85% (e.g., at least 86%, 90%, 95%, 96%, 97%, 98%, 99%, or more) sequence identity thereof, and exhibits greater than 50% (e.g., at least 50%, 55%, 60%, 65%, 70%, 75%, 80%, 85%, 90%, 91%, 92%, 93%, 94%, 95%, 96%, 97%, 98%, 99% or more) GluK2 knockdown.

Furthermore, the ASO agent may hybridize within at least a portion or region of exon 16 of a Grik2 mRNA, such as, e.g., exon 16 of the Grik2 mRNA situated at nucleotide positions 2856-4592 of SEQ ID NO: 115. The sequence of exon 16 of the Grik2 mRNA may be a nucleic acid sequence of SEQ ID NO: 144 or a variant thereof having at least 85% (e.g., at least 86%, 90%, 95%, 96%, 97%, 98%, 99%, or more) sequence identity to the nucleic acid sequence of SEQ ID NO: 144, as is shown in Table 4.

For example, Grik2 target nucleic acids contemplated for targeting using the ASO agents disclosed herein include nucleotides 2632-2652 of SEQ ID NO: 116 or nucleotides 2925-2945 of SEQ ID NO: 115, nucleotides 3382-3402 of SEQ ID NO: 115, nucleotides 3792-3812 of SEQ ID NO: 115, nucleotides 3347-3367 of SEQ ID NO: 115, nucleotides 3605-3625 of SEQ ID NO: 115, nucleotides 2581-2601 of SEQ ID NO: 116 or nucleotides 2874-2893 SEQ ID NO: 115, nucleotides 2581-2601 of SEQ ID NO: 116 or nucleotides 2874-2893 of SEQ ID NO: 115, nucleotides 4289-4309 of SEQ ID NO: 115, nucleotides 4274-4293 of SEQ ID NO: 115, nucleotides 4274-4294 of SEQ ID NO: 115, nucleotides 4078-4098 of SEQ ID NO: 115, nucleotides 3037-3057 of SEQ ID NO: 115, nucleotides 4417-4437 of SEQ ID NO: 115, nucleotides 2601-2620 of SEQ ID NO: 116 or nucleotides 2894-2913 of SEQ ID NO: 115, nucleotides 2601-2621 of SEQ ID NO: 116 or nucleotides 2894-2914 of SEQ ID NO: 115, nucleotides 3479-3499 of SEQ ID NO: 115, and nucleotides 3085-3105 of SEQ ID NO: 115, or a fragment or portion thereof.

The Grik2 ASO that targets a nucleic acid within a portion or region of exon 16 of SEQ ID NO: 116 or SEQ ID NO: 115 is selected from siRNA G4 (SEQ ID NO: 9), siRNA TS (SEQ ID NO: 10), siRNA TR (SEQ ID NO: 11), siRNA G(SEQ ID NO: 15), siRNA TN (SEQ ID NO: 16), siRNA G6 (SEQ ID NO: 18), siRNA G7 (SEQ ID NO: 19), siRNA GJ (SEQ ID NO: 27), siRNA CN (SEQ ID NO: 32), siRNA CO (SEQ ID NO: 33), siRNA CW (SEQ ID NO: 41), siRNA XS (SEQ ID NO: 49), siRNA XT (SEQ ID NO: 50), siRNA XV (SEQ ID NO: 52), siRNA XW (SEQ ID NO: 53), siRNA Y6 (SEQ ID NO: 61), or siRNA YA (SEQ ID NO: 63), or an ASO having greater than 85% (e.g., at least 86%, 90%, 95%, 96%, 97%, 98%, 99%, or more) sequence identity to siRNA G4 (SEQ ID NO: 9), siRNA TS (SEQ ID NO: 10), siRNA TR (SEQ ID NO: 11), siRNA G(SEQ ID NO: 15), siRNA TN (SEQ ID NO: 16), siRNA G6 (SEQ ID NO: 18), siRNA G7 (SEQ ID NO: 19), siRNA GJ (SEQ ID NO: 27), siRNA CN (SEQ ID NO: 32), siRNA CO (SEQ ID NO: 33), siRNA CW (SEQ ID NO: 41), siRNA XS (SEQ ID NO: 49), siRNA XT (SEQ ID NO: 50), siRNA XV (SEQ ID NO: 52), siRNA XW (SEQ ID NO: 53), siRNA Y6 (SEQ ID NO: 61), or siRNA YA (SEQ ID NO: 63). Additionally, the Grik2 ASO that targets a nucleic acid within a portion or region of exon 16 of SEQ ID NO: 116 or SEQ ID NO: 115 exhibits greater than 5% (e.g., at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown. Further still, the Grik2 ASO is selected from siRNA G4 (SEQ ID NO: 9), siRNA TS (SEQ ID NO: 10), siRNA TR (SEQ ID NO: 11), siRNA G(SEQ ID NO: 15), siRNA TN (SEQ ID NO: 16), siRNA G6 (SEQ ID NO: 18), siRNA G7 (SEQ ID NO: 19), siRNA GJ (SEQ ID NO: 27), siRNA CN (SEQ ID NO: 32), siRNA CO (SEQ ID NO: 33), siRNA CW (SEQ ID NO: 41), siRNA XS (SEQ ID NO: 49), siRNA XT (SEQ ID NO: 50), siRNA XV (SEQ ID NO: 52), siRNA XW (SEQ ID NO: 53), siRNA Y6 (SEQ ID NO: 61), or siRNA YA (SEQ ID NO: 63), or an ASO having greater than 85% (e.g., at least 86%, 90%, 95%, 96%, 97%, 98%, 99%, or more) sequence identity thereof, and exhibits greater than 5% (e.g., at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more) GluK2 knockdown.

### Thermodynamic Properties of Antisense Oligonucleotides and Grik2 Target Regions

RNA secondary structures, such as, e.g., those formed by the antisense agents of the present disclosure or the corresponding regions of a target sequence to which they hybridize (e.g., a Grik2 target sequence) can be described using concepts borrowed from thermodynamics, such as entropy and thermodynamic free energy. Thermodynamic free energy is generally described as the maximal amount of work that a system can perform in a process at constant temperature and signifies if the process is thermodynamically favorable or prohibitive. Put simply, thermodynamic free energy refers to the ability of a system to undergo a change in physical state. Within the context of a polynucleotide (e.g., an ASO agent of the disclosure or a substantially complementary sequence thereof), metrics based on thermodynamic free energy may describe the ease with which a particular secondary structure can be resolved (i.e., the energy required to open a secondary RNA structure of either the antisense oligonucleotide or its partial or full complement), the energy generated from duplex formation between or within RNA molecules, and the total energy of binding of an RNA molecule to itself or another RNA molecule, which takes into account the total energy required to resolve each RNA and the energy of the hybridization per se.

The present disclosure is based, in part, on the discovery made by the present inventors that thermodynamic characteristics of RNA molecules (e.g., ASO constructs of the disclosure or substantially complementary sequences thereof, such as, e.g., a Grik2 target region) may be used to predict the efficacy with which an antisense molecule can knockdown the expression of a target mRNA. Accordingly, the compositions and methods disclosed herein may characterize an ASO sequence or its target mRNA sequence using thermodynamic parameters to predict the likelihood of knockdown of mRNA expression.

In particular, the present disclosure provides three distinct thermodynamic parameters that are useful in predicting the knockdown efficacy of a particular ASO sequence with respect to its target mRNA region, namely Total Free Energy of Binding, Energy from Duplex Formation, and Target Opening Energy (or Opening Energy). An additional concept that may be used to characterize the thermodynamic stability of an RNA molecule and to predict the knockdown efficacy of a particular ASO agent is the GC (Guanine-Cytosine; %) content of an RNA molecule. Within the context of the present disclosure, Total Free Energy of Binding (kcal/mol) of an ASO refers to the free energy of the process of the ASO hybridizing to its corresponding target mRNA sequence. This includes the energy required to open the target region of the mRNA (e.g., a Grik2 mRNA), the energy required to generate a single-stranded antisense guide sequence, and energy of hybridization between the polynucleotide and its complement (full or substantial). Relatedly, Energy from Duplex Formation refers to a thermodynamic property that indicates the favorability of the formation of a duplex structure between two RNA molecules, and, resultantly, the stability of the RNA duplex. Total Opening Energy is a thermodynamic metric that reflects the energy required to resolve (i.e., open/render accessible) an RNA secondary structure at the target location, including resolution of nearby secondary structures or involvement of distal sequences that form a secondary structure with the target sequence.

Accordingly, the present disclosure contemplates ASO sequences (such as, e.g., the ASO sequences disclosed herein) having a Total Opening Energy that is less than kcal/mol (e.g., less than kcal/mol, 9 kcal/mol, 8 kcal/mol, 7 kcal/mol, 6 kcal/mol, kcal/mol, 4 kcal/mol, 3 kcal/mol, 2 kcal/mol, or 1 kcal/mol). In a particular example, the ASO sequence of the disclosure has a Total Opening Energy that is less than 9 kcal/mol (e.g., less than 8 kcal/mol, 7 kcal/mol, 6 kcal/mol, kcal/mol, 4 kcal/mol, 3 kcal/mol, 2 kcal/mol, or 1 kcal/mol, or less). In another example, the ASO sequence of the disclosure has a Total Opening Energy that is less than 8 kcal/mol (e.g., less than 7 kcal/mol, 6 kcal/mol, kcal/mol, 4 kcal/mol, 3 kcal/mol, 2 kcal/mol, or 1 kcal/mol, or less). In another example, the ASO sequence of the disclosure has a Total Opening Energy that is less than 7 kcal/mol (e.g., less than 6 kcal/mol, kcal/mol, 4 kcal/mol, 3 kcal/mol, 2 kcal/mol, or 1 kcal/mol, or less). In another example, the ASO sequence of the disclosure has a Total Opening Energy that is less than 6 kcal/mol (e.g., less than kcal/mol, 4 kcal/mol, 3 kcal/mol, 2 kcal/mol, or 1 kcal/mol, or less). In another example, the ASO sequence of the disclosure has a Total Opening Energy that is less than kcal/mol (e.g., less than 4 kcal/mol, 3 kcal/mol, 2 kcal/mol, or 1 kcal/mol, or less). In another example, the ASO sequence of the disclosure has a Total Opening Energy that is less than 4 kcal/mol (e.g., less than 3 kcal/mol, 2 kcal/mol, or 1 kcal/mol, or less). In another example, the ASO sequence of the disclosure has a Total Opening Energy that is less than 3 kcal/mol (e.g., less than 2 kcal/mol, or 1 kcal/mol, or less). In another example, the ASO sequence of the disclosure has a Total Opening Energy that is less than 2 kcal/mol (e.g., 1 kcal/mol, or less). In another example, the ASO sequence of the disclosure has a Total Opening Energy that is less than 1 kcal/mol.

Furthermore, disclosed herein are ASO sequences having an Energy of/from Duplex Formation that is greater than −41 kcal/mol, (e.g., greater than −kcal/mol, −38 kcal/mol, −kcal/mol, −kcal/mol, −kcal/mol, −kcal/mol, −kcal/mol, −kcal/mol, −kcal/mol, −4 kcal/mol, −3 kcal/mol, −2 kcal/mol, −1 kcal/mol or greater). In other examples, the ASO sequence of the disclosure has an Energy from Duplex Formation that is greater than −38 kcal/mol (e.g., −kcal/mol, −kcal/mol, −kcal/mol, −kcal/mol, −kcal/mol, −kcal/mol, −kcal/mol, −4 kcal/mol, −3 kcal/mol, −2 kcal/mol, −1 kcal/mol or greater). In some examples, the ASO sequence of the disclosure has an Energy from Duplex Formation that is greater than −kcal/mol (e.g., greater than −kcal/mol, −kcal/mol, −kcal/mol, −kcal/mol, −kcal/mol, −kcal/mol, −4 kcal/mol, −3 kcal/mol, −2 kcal/mol, −1 kcal/mol or greater). In a particular example, the ASO sequence of the disclosure has an Energy from Duplex Formation that is greater than −kcal/mol (e.g., greater than −kcal/mol, −kcal/mol, −kcal/mol, −kcal/mol, −kcal/mol, −4 kcal/mol, −3 kcal/mol, −2 kcal/mol, −1 kcal/mol or greater). In another example, the ASO sequence of the disclosure has an Energy from Duplex Formation that is greater than −kcal/mol (e.g., greater than −kcal/mol, −kcal/mol, −kcal/mol, −kcal/mol, −4 kcal/mol, −3 kcal/mol, −2 kcal/mol, −1 kcal/mol or greater). In another example, the ASO sequence of the disclosure has an Energy from Duplex Formation that is greater than −kcal/mol (e.g., greater than −kcal/mol, −kcal/mol, −kcal/mol, −4 kcal/mol, −3 kcal/mol, −2 kcal/mol, −1 kcal/mol or greater). In another example, the ASO sequence of the disclosure has an Energy from Duplex Formation that is greater than −kcal/mol (e.g., greater than −kcal/mol, −kcal/mol, −4 kcal/mol, −3 kcal/mol, −2 kcal/mol, −1 kcal/mol or greater). In another example, the ASO sequence of the disclosure has an Energy from Duplex Formation that is greater than −kcal/mol (e.g., greater than −kcal/mol, −4 kcal/mol, −3 kcal/mol, −2 kcal/mol, −1 kcal/mol or greater). In another example, the ASO sequence of the disclosure has an Energy from Duplex Formation that is greater than −5 kcal/mol (e.g., greater than −4 kcal/mol, −3 kcal/mol, −2 kcal/mol, −1 kcal/mol or greater). In another example, the ASO sequence of the disclosure has an Energy from Duplex Formation that is greater than −4 kcal/mol (e.g., greater than −3 kcal/mol, −2 kcal/mol, −1 kcal/mol or greater). In another example, the ASO sequence of the disclosure has an Energy from Duplex Formation that is greater than −3 kcal/mol (e.g., greater than −3 kcal/mol, −2 kcal/mol, −1 kcal/mol or greater). In another example, the ASO sequence of the disclosure has an Energy from Duplex Formation that is greater than −2 kcal/mol (e.g., greater than −2 kcal/mol, −1 kcal/mol, or greater). In another example, the ASO sequence of the disclosure has an Energy from Duplex Formation that is greater than −1 kcal/mol.

Additionally, the present disclosure further relates to an ASO sequence having an Total Free Energy of Binding that is greater than −30.5 kcal/mol (e.g., greater than −27 kcal/mol, −24 kcal/mol, −20 kcal/mol, −kcal/mol, −kcal/mol, −kcal/mol, −4 kcal/mol, −3 kcal/mol, −2 kcal/mol, −1 kcal/mol, or greater). In some examples, the ASO sequence of the disclosure has an Total Free Energy of Binding that is greater than −27 kcal/mol (e.g., greater than −24 kcal/mol, −kcal/mol, −kcal/mol, −kcal/mol, −kcal/mol, −4 kcal/mol, −3 kcal/mol, −2 kcal/mol, −1 kcal/mol or greater). In yet another example, the ASO sequence of the disclosure has an Total Free Energy of Binding that is greater than −24 kcal/mol (e.g., greater than −kcal/mol, −kcal/mol, −kcal/mol, −kcal/mol, −4 kcal/mol, −3 kcal/mol, −2 kcal/mol, −1 kcal/mol or greater). In another example, the ASO sequence of the disclosure has an Total Free Energy of Binding that is greater than −kcal/mol (e.g., greater than −kcal/mol, −10 kcal/mol, −kcal/mol, −4 kcal/mol, −3 kcal/mol, −2 kcal/mol, −1 kcal/mol or greater). In another example, the ASO sequence of the disclosure has an Total Free Energy of Binding that is greater than −kcal/mol (e.g., greater than −kcal/mol, −kcal/mol, −4 kcal/mol, −3 kcal/mol, −2 kcal/mol, −1 kcal/mol or greater). In another example, the ASO sequence of the disclosure has an Total Free Energy of Binding that is greater than −kcal/mol (e.g., greater than −kcal/mol, −4 kcal/mol, −3 kcal/mol, −2 kcal/mol, −1 kcal/mol or greater). In another example, the ASO sequence of the disclosure has an Total Free Energy of Binding that is greater than −kcal/mol (e.g., greater than −4 kcal/mol, −3 kcal/mol, −2 kcal/mol, −1 kcal/mol or greater). In another example, the ASO sequence of the disclosure has an Total Free Energy of Binding that is greater than −4 kcal/mol (e.g., greater than −3 kcal/mol, −2 kcal/mol, −1 kcal/mol or greater).

In another example, the ASO sequence of the disclosure has an Total Free Energy of Binding that is greater than −3 kcal/mol (e.g., greater than −3 kcal/mol, −2 kcal/mol, −1 kcal/mol or greater). In another example, the ASO sequence of the disclosure has an Total Free Energy of Binding that is greater than −2 kcal/mol (e.g., greater than −2 kcal/mol, −1 kcal/mol, or greater). In another example, the ASO sequence of the disclosure has an Total Free Energy of Binding that is greater than −1 kcal/mol.

Moreover, the present disclosure also contemplates an ASO sequence having a GC content that is less than 60% (e.g., less than 55%, 50%, 45%, 40%, 35%, 30%, 25%, 20%, 15%, 10%, 5%, 4%, 3%, 2%, 1%, or less). In other examples, the ASO sequence has a GC content that is less than 55% (e.g., less than 50%, 45%, 40%, 35%, 30%, 25%, 20%, 15%, 10%, 5%, 4%, 3%, 2%, 1%, or less). In still other examples, the ASO sequence has a GC content that is less than 50% (e.g., less than 45%, 40%, 35%, 30%, 25%, 20%, 15%, 10%, 5%, 4%, 3%, 2%, 1%, or less). In a particular example, the ASO sequence has a GC content that is less than 45% (e.g., less than 40%, 35%, 30%, 25%, 20%, 15%, 10%, 5%, 4%, 3%, 2%, 1%, or less). In another example, the ASO sequence has a GC content that is less than 40% (e.g., less than 35%, 30%, 25%, 20%, 15%, 10%, 5%, 4%, 3%, 2%, 1%, or less). In another example, the ASO sequence has a GC content that is less than 35% (e.g., less than 30%, 25%, 20%, 15%, 10%, 5%, 4%, 3%, 2%, 1%, or less). In another example, the ASO sequence has a GC content that is less than 30% (e.g., less than 25%, 20%, 15%, 10%, 5%, 4%, 3%, 2%, 1%, or less). In another example, the ASO sequence has a GC content that is less than 35% (e.g., less than 30%, 25%, 20%, 15%, 10%, 5%, 4%, 3%, 2%, 1%, or less). In another example, the ASO sequence has a GC content that is less than 25% (e.g., less than 20%, 15%, 10%, 5%, 4%, 3%, 2%, 1%, or less). In another example, the ASO sequence has a GC content that is less than 20% (e.g., less than 15%, 10%, 5%, 4%, 3%, 2%, 1%, or less). In another example, the ASO sequence has a GC content that is less than 15% (e.g., less than 10%, 5%, 4%, 3%, 2%, 1%, or less). In another example, the ASO sequence has a GC content that is less than 10% (e.g., less than 5%, 4%, 3%, 2%, 1%, or less). In another example, the ASO sequence has a GC content that is less than 5% (e.g., less than 4%, 3%, 2%, 1%, or less). In another example, the ASO sequence has a GC content that is less than 4% (e.g., less than 3%, 2%, 1%, or less). In another example, the ASO sequence has a GC content that is less than 3% (e.g., less than 2%, 1%, or less). In another example, the ASO sequence has a GC content that is less than 2% (e.g., less 1%, or less). In another example, the ASO sequence has a GC content that is less than 1%.

Methods of determining thermodynamic characteristics of a biomolecule, such as an RNA molecule (e.g., an ASO RNA molecule of the disclosure or a substantially complementary sequence thereof) are well-known in the art. For example, Gruber et al. (Nucleic Acids Research 36:W70-4, 2008) summarize a collection of tools that can be used for the design of RNA sequences and analysis of folding and thermodynamic characteristics of RNA molecules. The disclosure of Gruber et al. is incorporated by reference herein as it relates to methods of determining thermodynamic properties of RNA molecules.

Grik2 mRNA Secondary Structures

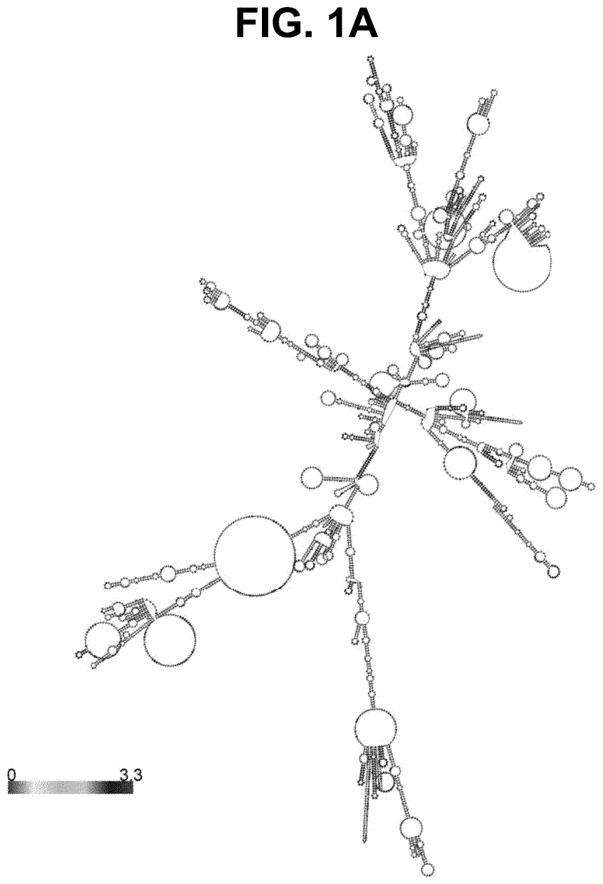

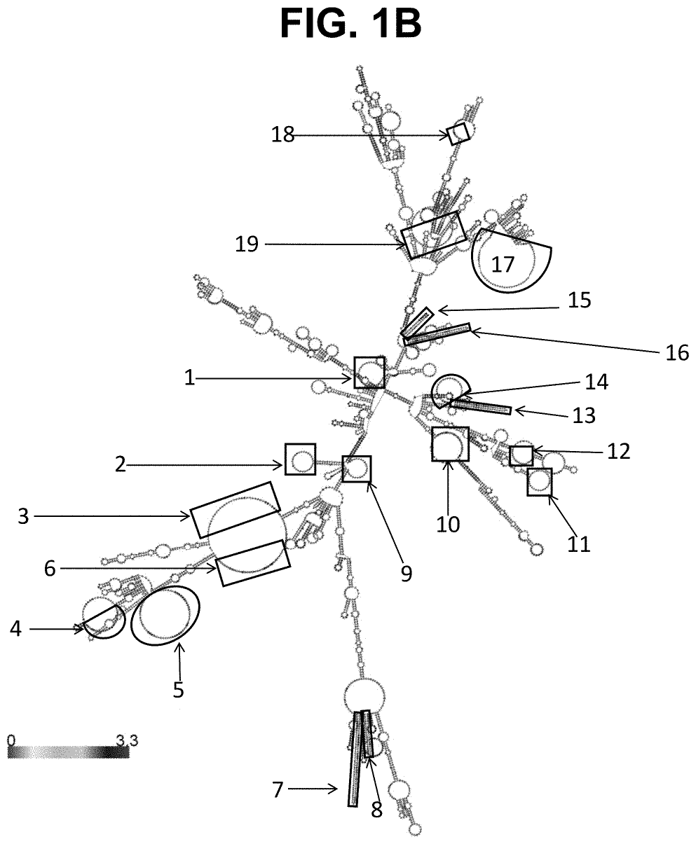

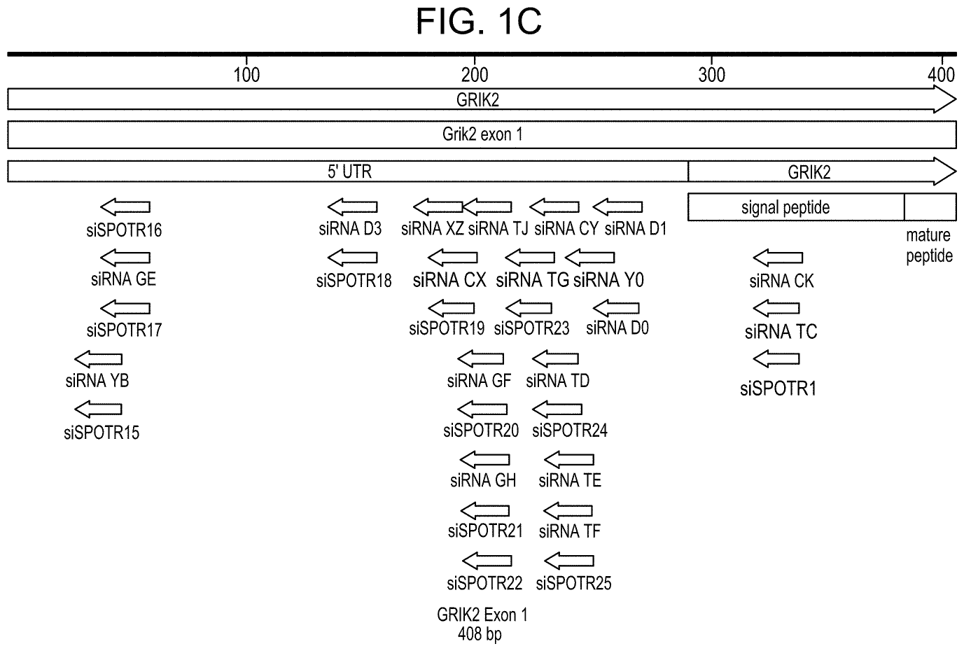

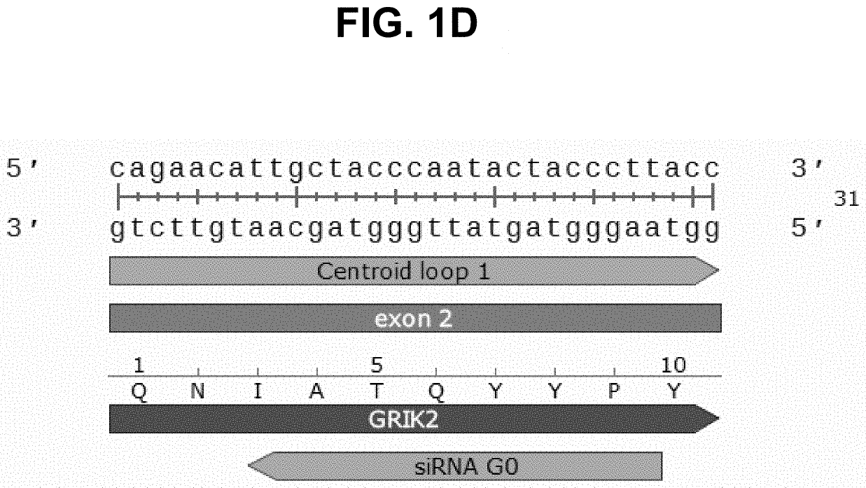

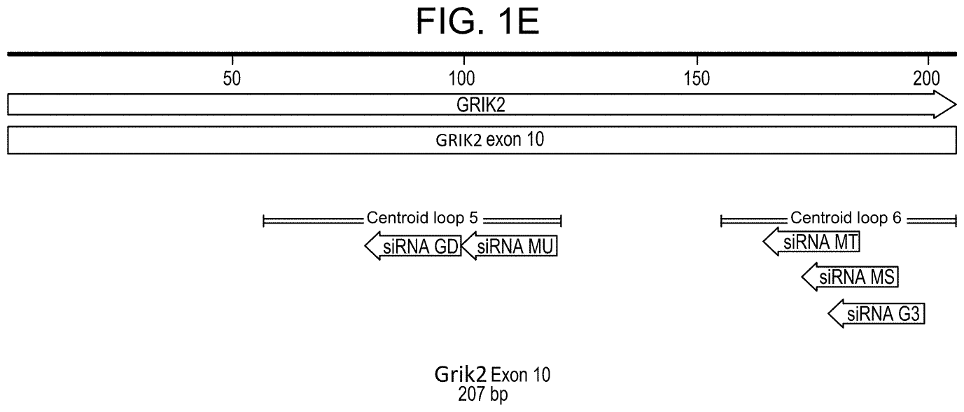

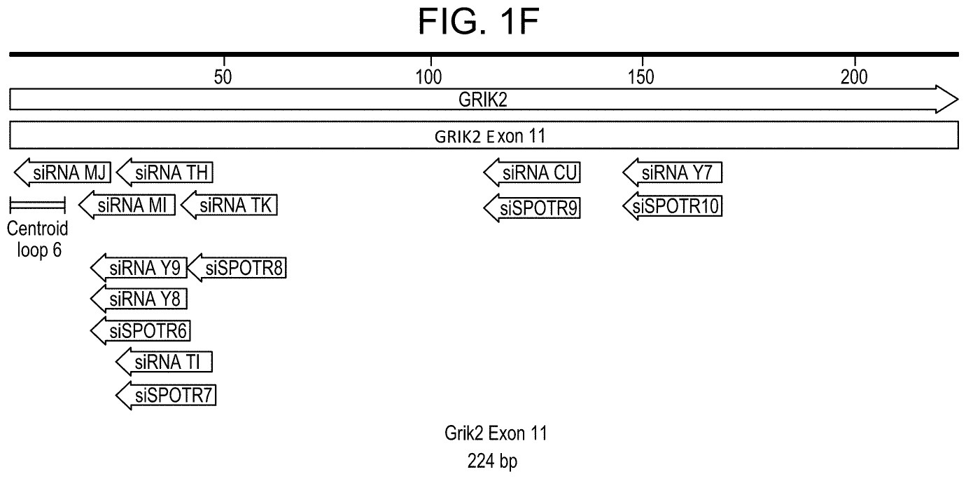

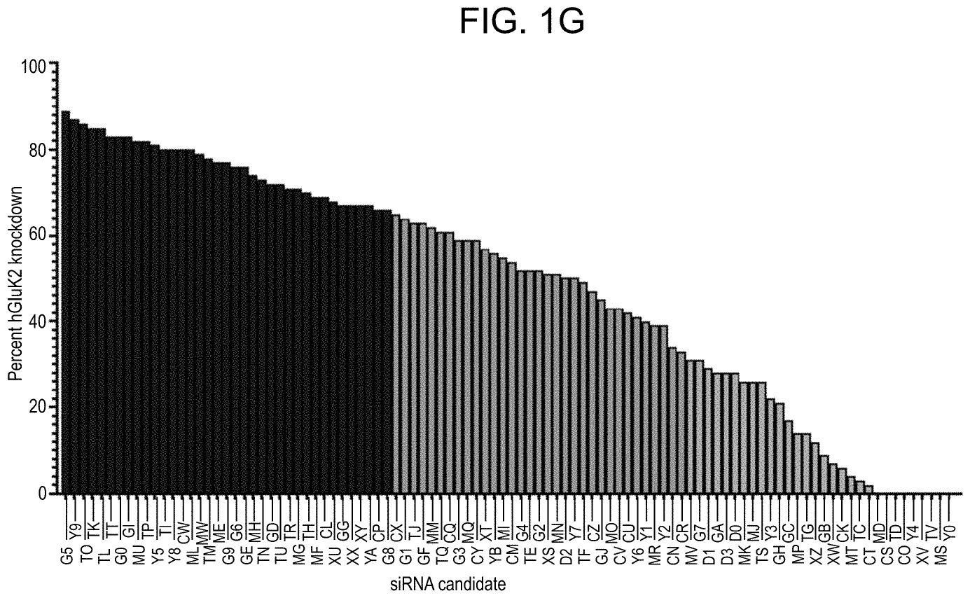

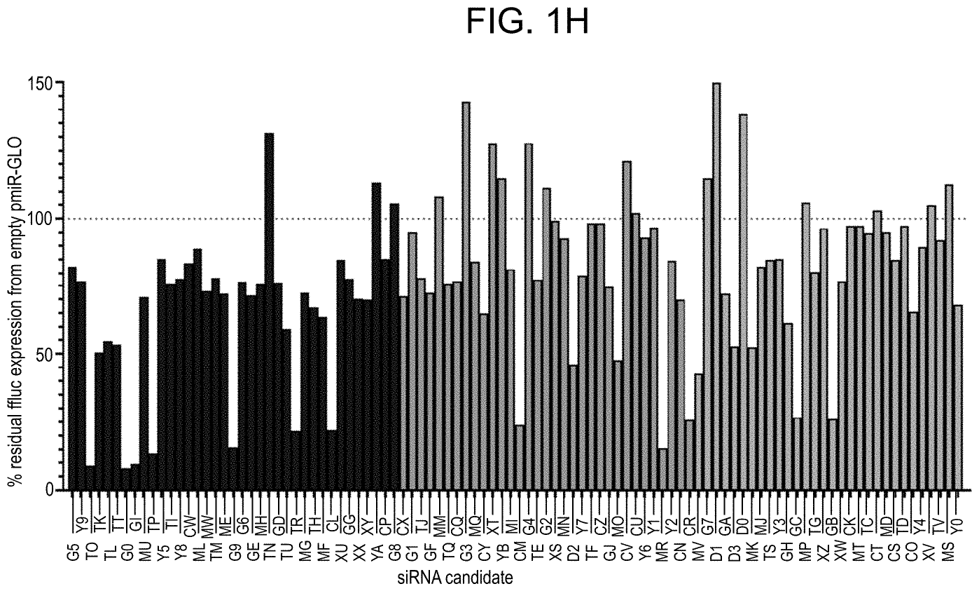

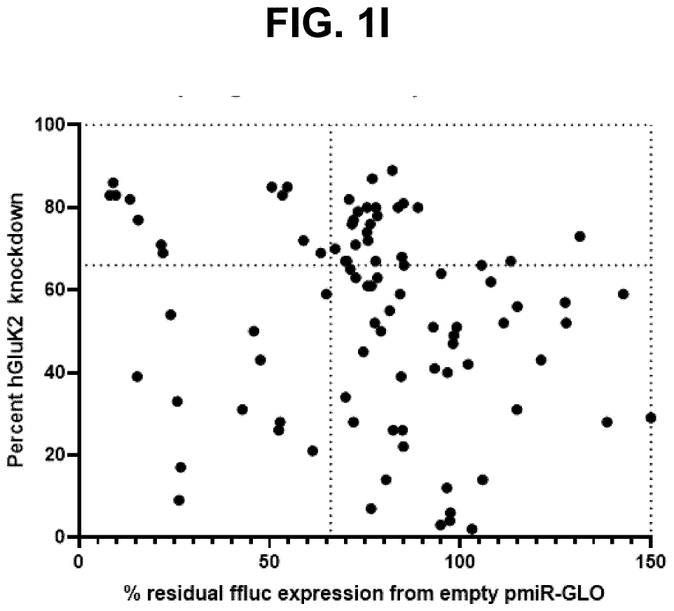

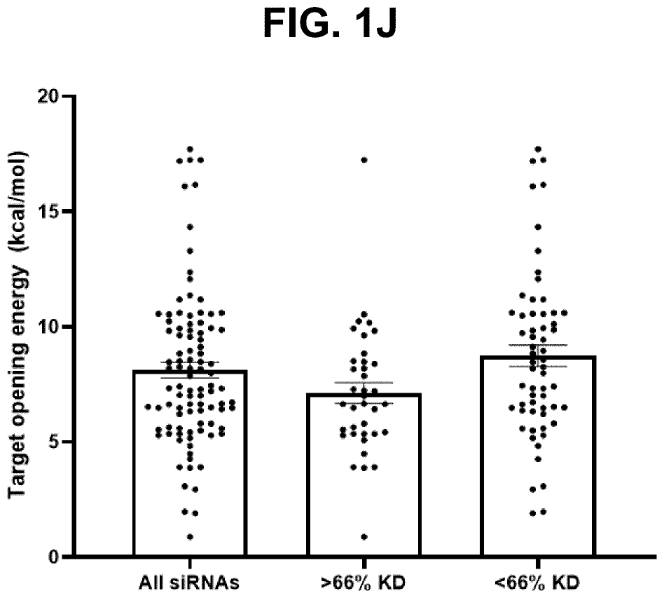

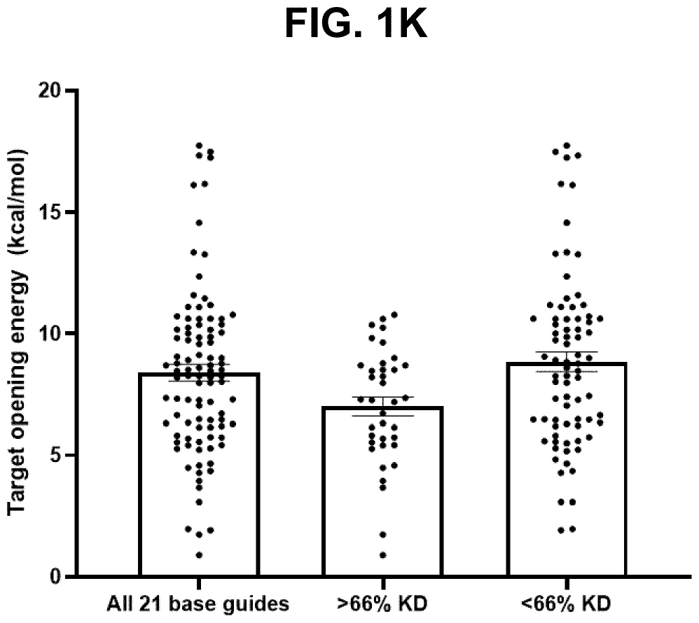

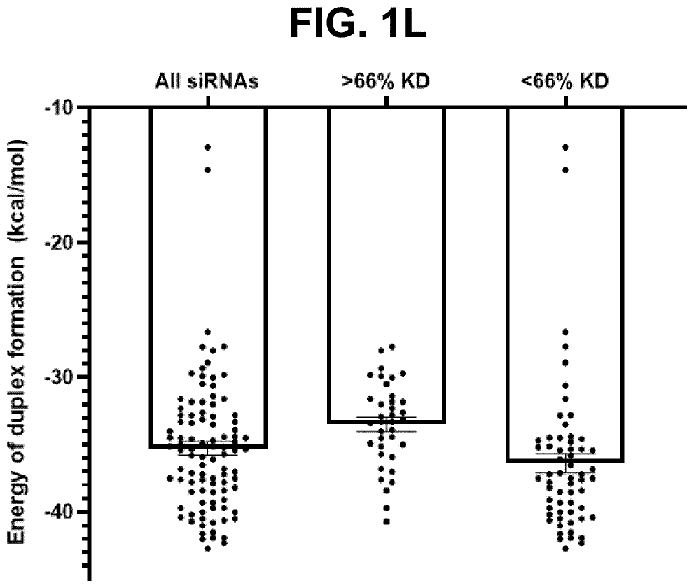

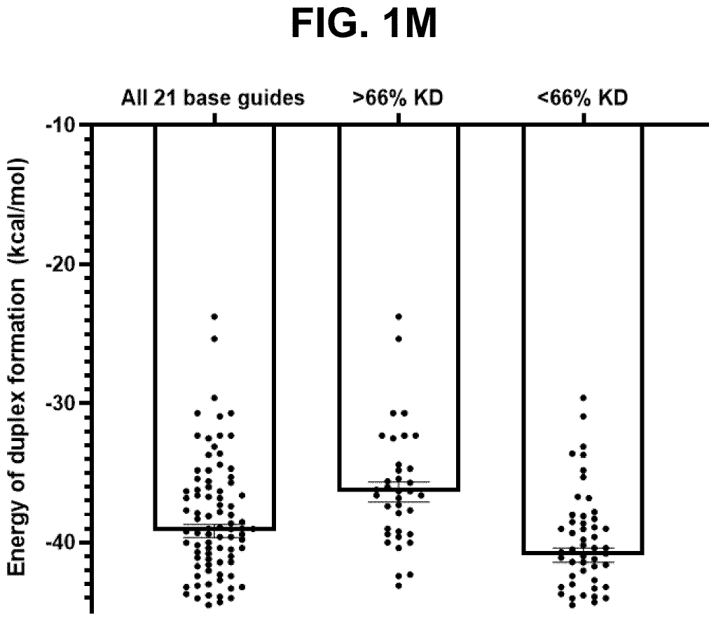

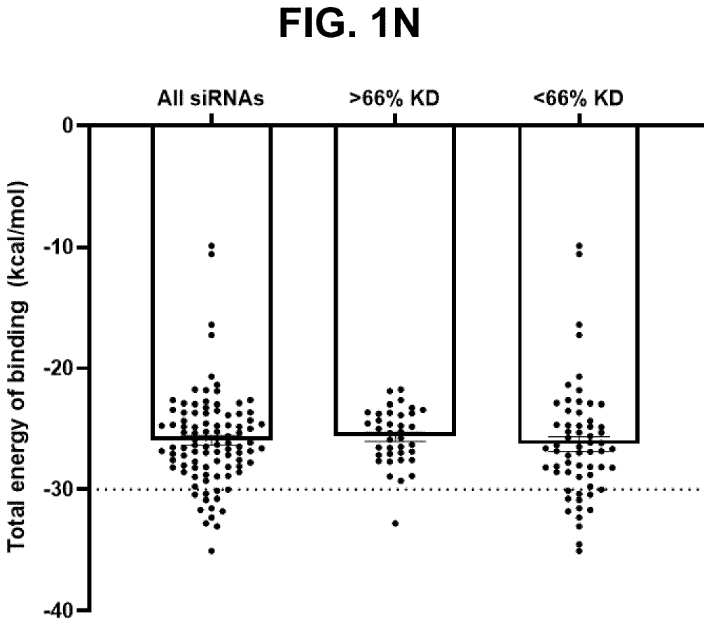

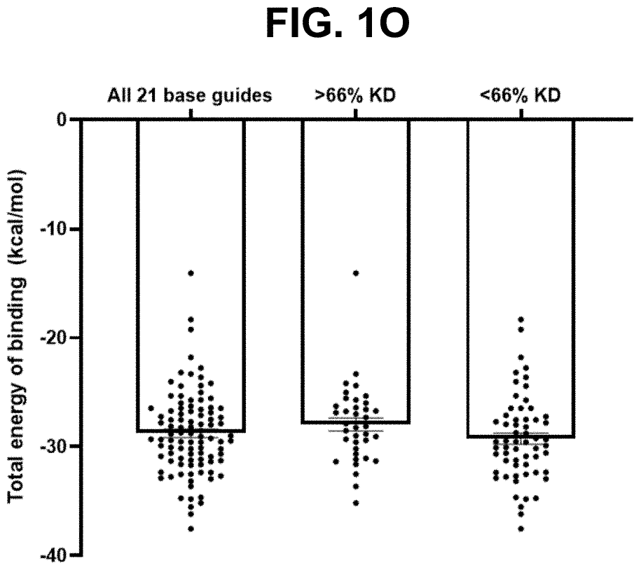

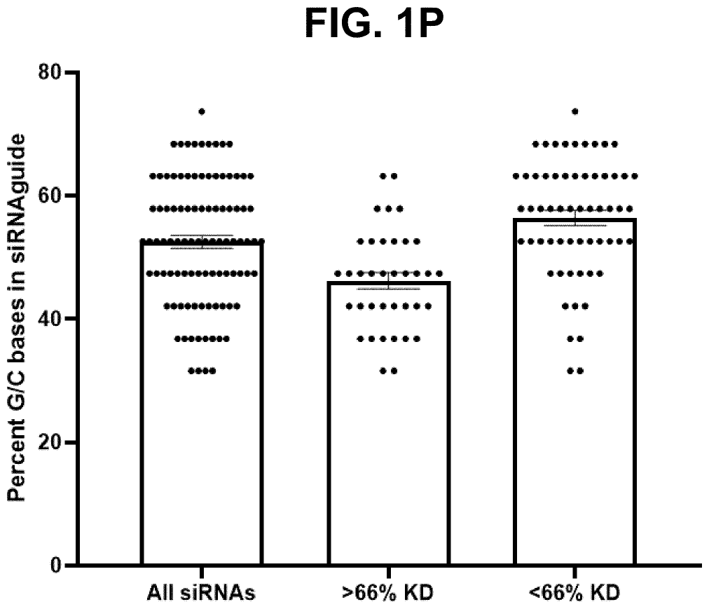

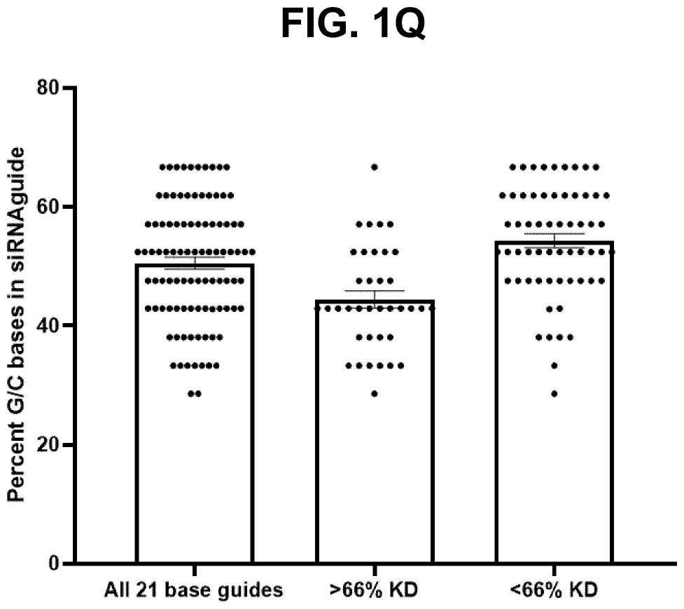

RNA-Induced Silencing Complex (RISC) is a ribonucleoprotein particle composed of single-stranded small RNAs (smRNA), including short interfering RNAs (siRNAs), and an endonucleolytically active argonaut protein, capable of cleaving mRNAs complementary to the smRNA (e.g., an ASO, such as, e.g., siRNA, shRNA, miRNA, or shmiRNA, or shmiRNA) (Pratt AJ, MacRae I J. The RNA-induced silencing complex: a versatile gene-silencing machine. J Biol Chem.; 284(27):17897 - 17901, 2009; which is incorporated herein in its entirety). It has been shown that RISC loading is influenced by a variety of factors that govern the degree of mRNA knockdown. Nucleotide sequences of the target mRNA and antisense sequence may contribute to poor RISC loading, duplex unwinding, and decreased specificity. Target site secondary structures may impact RISC—target annealing independently of smRNA-complementarity. Not wishing to be bound by theory, certain target site secondary structures of Grik2 transcripts determined to have low base pairing probability and/or high positional entropy (shaded with increasing intensity in the scale of FIGS. 1A and 1B; also see Example 1) were identified and prioritized for guide (e.g., antisense sequence) design. These regions included clearly delineated loop regions (“centroid loop,” or simply “loop”) as well as regions depicted as stem-like (“unpaired”) regions and each have low probability of base pairing within the secondary Grik2 mRNA structure. Priority was given to regions that were predicted to have low base pairing probability and/or high positional entropy in one or more species (human, at minimum, and in some cases in at least one more species Grik2 transcript, such as mouse or monkey). In fact, many loop and stem-like unpaired regions of the predicted secondary Grik2 mRNA structure (Table 4 and FIG. 1B) contain favorable regions that exhibit a low energy requirement, e.g. less than 10, and even less than 7.5 kcal/mol Target Opening Energy as determined by RNAup or equivalent calculation, that is favorable in a pairing arrangement with various siRNA and miRNA guides (See Example 1 B, Table 12, and Table 13).

As such, Grik2 target nucleic acids within the secondary structure portions or regions of Grik2 mRNA, e.g., loop and unpaired regions, have been identified that are capable of reducing expression of Grik2 when hybridized to an ASO agent of the disclosure (e.g., any one of SEQ ID NOs: 1-108), and are embodiments of the invention. See Table 4 and FIG. 1B for exemplary secondary structure regions within the Grik2 mRNA.

Accordingly, the disclosed ASO agents may bind to a secondary structure (e.g., a loop or unpaired secondary structure) within the Grik2 mRNA. For example, the ASO agents may bind to a loop region within the secondary structure of the Grik2 mRNA, such as, e.g., a loop1 region located at nucleotide positions 494-524 of SEQ ID NO: 115 or positions 201-231 of SEQ ID NO: 116. The loop1 region may have a nucleic acid sequence of SEQ ID NO: 145, or may be a variant thereof having at least 85% (e.g., at least 86%, 90%, 95%, 96%, 97%, 98%, 99%, or more) sequence identity to the nucleic acid sequence of SEQ ID NO: 145, as is shown in Table 4. Accordingly, the disclosed ASO agents may bind within at least a portion of loop1 region of a Grik2 mRNA. For example, a Grik2 ASO agent that targets a nucleic acid sequence within a portion or region of loop1 region (SEQ ID NO: 145) of SEQ ID NO: 115 or 116 may be siRNA GO (SEQ ID NO: 1) or a variant thereof having at least 85% (e.g., at least 86%, 90%, 95%, 96%, 97%, 98%, 99%, or more) sequence identity to the nucleic acid sequence of siRNA GO (SEQ ID NO: 1).

In other cases, the ASO agent may bind to a loop2 region located at nucleotide positions 1098-1124 of SEQ ID NO: 115 or positions 805-831 of SEQ ID NO: 116. The loop2 region may have a nucleic acid sequence of SEQ ID NO: 146, or may be a variant thereof having at least 85% (e.g., at least 86%, 90%, 95%, 96%, 97%, 98%, 99%, or more) sequence identity to the nucleic acid sequence of SEQ ID NO: 146, as is shown in Table 4. Accordingly, the disclosed ASO agents may bind within at least a portion of loop2 region of a Grik2 mRNA. For example, a Grik2 ASO agent that targets a nucleic acid sequence within a portion or region of loop2 region (SEQ ID NO: 146) of SEQ ID NO: 115 or 116 may be siRNA TT (SEQ ID NO: 4) or a variant thereof having at least 85% (e.g., at least 86%, 90%, 95%, 96%, 97%, 98%, 99%, or more) sequence identity to the nucleic acid sequence of siRNA TT (SEQ ID NO: 4).

The ASO agent may also be one that binds to a loop3 region located at nucleotide positions 1197-1237 of SEQ ID NO: 115 or positions 904-944 of SEQ ID NO: 116. The loop3 region may have a nucleic acid sequence of SEQ ID NO: 147, or may be a variant thereof having at least 85% (e.g., at least 86%, 90%, 95%, 96%, 97%, 98%, 99%, or more) sequence identity to the nucleic acid sequence of SEQ ID NO: 147, as is shown in Table 4. Accordingly, the disclosed ASO agents may bind within at least a portion of loop3 region of a Grik2 mRNA. For example, a Grik2 ASO agent that targets a nucleic acid sequence within a portion or region of loop3 region (SEQ ID NO: 147) of SEQ ID NO: 115 or 116 may be siRNA G1 (SEQ ID NO: 5) or siRNA G2 (SEQ ID NO: 6) or a variant thereof having at least 85% (e.g., at least 86%, 90%, 95%, 96%, 97%, 98%, 99%, or more) sequence identity to the nucleic acid sequence of siRNA G1 (SEQ ID NO: 5) or siRNA G2 (SEQ ID NO: 6).

In an additional example, ASO agent may bind to a loop4 region located at nucleotide positions 1543-1569 of SEQ ID NO: 115 or positions 1250-1276 of SEQ ID NO: 116. The loop4 region may have a nucleic acid sequence of SEQ ID NO: 148, or may be a variant thereof having at least 85% (e.g., at least 86%, 90%, 95%, 96%, 97%, 98%, 99%, or more) sequence identity to the nucleic acid sequence of SEQ ID NO: 148, as is shown in Table 4. Accordingly, the disclosed ASO agents may bind within at least a portion of loop4 region of a Grik2 mRNA.

The ASO agent may also be one that binds to a loop region located at nucleotide positions 1667-1731 of SEQ ID NO: 115 or positions 1374-1438 of SEQ ID NO: 116. The loop region may have a nucleic acid sequence of SEQ ID NO: 149, or may be a variant thereof having at least 85% (e.g., at least 86%, 90%, 95%, 96%, 97%, 98%, 99%, or more) sequence identity to the nucleic acid sequence of SEQ ID NO: 149, as is shown in Table 4. Accordingly, the disclosed ASO agents may bind within at least a portion of loop region of a Grik2 mRNA. For example, a Grik2 ASO agent that targets a nucleic acid sequence within a portion or region of loop region (SEQ ID NO: 149) of SEQ ID NO: 115 or 116 may be siRNA GD (SEQ ID NO: 7) or siRNA MU (SEQ ID NO: 96) or a variant thereof having at least 85% (e.g., at least 86%, 90%, 95%, 96%, 97%, 98%, 99%, or more) sequence identity to the nucleic acid sequence of siRNA GD (SEQ ID NO: 7) or siRNA MU (SEQ ID NO: 96).

In additional examples, the ASO agent may be one that binds to a loop6 region located at nucleotide positions 1767-1830 of SEQ ID NO: 115 or positions 1474-1537 of SEQ ID NO: 116. The loop 6 region may have a nucleic acid sequence of SEQ ID NO: 150, or may be a variant thereof having at least 85% (e.g., at least 86%, 90%, 95%, 96%, 97%, 98%, 99%, or more) sequence identity to the nucleic acid sequence of SEQ ID NO: 150, as is shown in Table 4. Accordingly, the disclosed ASO agents may bind within at least a portion of loop6 region of a Grik2 mRNA. For example, a Grik2 ASO agent that targets a nucleic acid sequence within a portion or region of loop6 region (SEQ ID NO: 150) of SEQ ID NO: 115 or 116 may be siRNA G3 (SEQ ID NO: 8), siRNA MS (SEQ ID NO: 99), or siRNA MT (SEQ ID NO: 98), or a variant thereof having at least 85% (e.g., at least 86%, 90%, 95%, 96%, 97%, 98%, 99%, or more) sequence identity to the nucleic acid sequence of siRNA G3 (SEQ ID NO: 8), siRNA MS (SEQ ID NO: 99), or siRNA MT (SEQ ID NO: 98).

The ASO agent may also be one that binds to a loop7 region located at nucleotide positions 2693-2716 of SEQ ID NO: 115 or positions 2400-2423 of SEQ ID NO: 116. The loop7 region may have a nucleic acid sequence of SEQ ID NO: 151, or is a variant thereof having at least 85% (e.g., at least 86%, 90%, 95%, 96%, 97%, 98%, 99%, or more) sequence identity to the nucleic acid sequence of SEQ ID NO: 151, as is shown in Table 4. Accordingly, the disclosed ASO agents may bind within at least a portion of loop7 region of a Grik2 mRNA.

The ASO agent may also be one that binds to a loop8 region located at nucleotide positions 2916-2955 of SEQ ID NO: 115 or positions 2623-2662 of SEQ ID NO: 116. The loop8 region may have a nucleic acid sequence of SEQ ID NO: 152, or may be a variant thereof having at least 85% (e.g., at least 86%, 90%, 95%, 96%, 97%, 98%, 99%, or more) sequence identity to the nucleic acid sequence of SEQ ID NO: 152, as is shown in Table 4. Accordingly, the disclosed ASO agents may bind within at least a portion of loop8 region of a Grik2 mRNA. For example, a Grik2 ASO agent that targets a nucleic acid sequence within a portion or region of loop8 region (SEQ ID NO: 152) of SEQ ID NO: 115 or 116 may be siRNA G4 (SEQ ID NO: 9) or a variant thereof having at least 85% (e.g., at least 86%, 90%, 95%, 96%, 97%, 98%, 99%, or more) sequence identity to the nucleic acid sequence of siRNA G4 (SEQ ID NO: 9).

Additionally, the ASO agent may be one that binds to a loop9 region located at nucleotide positions 3065-3091 of SEQ ID NO: 115. The loop9 region may have a nucleic acid sequence of SEQ ID NO: 153, or may be a variant thereof having at least 85% (e.g., at least 86%, 90%, 95%, 96%, 97%, 98%, 99%, or more) sequence identity to the nucleic acid sequence of SEQ ID NO: 153, as is shown in Table 4. Accordingly, the disclosed ASO agents may bind within at least a portion of loop9 region of a Grik2 mRNA. For example, a Grik2 ASO agent that targets a nucleic acid sequence within a portion or region of loop9 region (SEQ ID NO: 153) of SEQ ID NO: 115 or 116 may be siRNA YA (SEQ ID NO: 63) or a variant thereof having at least 85% (e.g., at least 86%, 90%, 95%, 96%, 97%, 98%, 99%, or more) sequence identity to the nucleic acid sequence of siRNA YA (SEQ ID NO: 63).

Furthermore, the ASO agent may be one that binds to a loop region located at nucleotide positions 3141-3163 of SEQ ID NO: 115. The loop region may have a nucleic acid sequence of SEQ ID NO: 154, or may be a variant thereof having at least 85% (e.g., at least 86%, 90%, 95%, 96%, 97%, 98%, 99%, or more) sequence identity to the nucleic acid sequence of SEQ ID NO: 154, as is shown in Table 4. Accordingly, the disclosed ASO agents may bind within at least a portion of loop region of a Grik2 mRNA.

In a further example, the ASO agent may be one that binds to a loop11 region located at nucleotide positions 3382-3413 of SEQ ID NO: 115. The loop11 region may have a nucleic acid sequence of SEQ ID NO: 155, or may be a variant thereof having at least 85% (e.g., at least 86%, 90%, 95%, 96%, 97%, 98%, 99%, or more) sequence identity to the nucleic acid sequence of SEQ ID NO: 155, as is shown in Table 4. Accordingly, the disclosed ASO agents may bind within at least a portion of loop 11 region of a Grik2 mRNA. For example, a Grik2 ASO agent that targets a nucleic acid sequence within a portion or region of loop11 region (SEQ ID NO: 155) of SEQ ID NO: 115 or 116 may be siRNA TS (SEQ ID NO: 10) or a variant thereof having at least 85% (e.g., at least 86%, 90%, 95%, 96%, 97%, 98%, 99%, or more) sequence identity to the nucleic acid sequence of siRNA TS (SEQ ID NO: 10).

The ASO agent may also be one that binds to a loop12 region located at nucleotide positions 3788-3856 of SEQ ID NO: 115. The loop12 region may have a nucleic acid sequence of SEQ ID NO: 156, or may be a variant thereof having at least 85% (e.g., at least 86%, 90%, 95%, 96%, 97%, 98%, 99%, or more) sequence identity to the nucleic acid sequence of SEQ ID NO: 156, as is shown in Table 4. Accordingly, the disclosed ASO agents may bind within at least a portion of loop12 region of a Grik2 mRNA. For example, a Grik2 ASO agent that targets a nucleic acid sequence within a portion or region of loop2 region (SEQ ID NO: 156) of SEQ ID NO: 115 or 116 may be siRNA TR (SEQ ID NO: 11) or a variant thereof having at least 85% (e.g., at least 86%, 90%, 95%, 96%, 97%, 98%, 99%, or more) sequence identity to the nucleic acid sequence of siRNA TR (SEQ ID NO: 11).

The ASO agent may be one that binds to a loop13 region located at nucleotide positions 4550-4592 of SEQ ID NO: 115. The loop13 region may have a nucleic acid sequence of SEQ ID NO: 157, or may be a variant thereof having at least 85% (e.g., at least 86%, 90%, 95%, 96%, 97%, 98%, 99%, or more) sequence identity to the nucleic acid sequence of SEQ ID NO: 157, as is shown in Table 4. Accordingly, the disclosed ASO agents may bind within at least a portion of loop13 region of a Grik2 mRNA.

In a further example, the ASO agent may be one that binds to a loop14 region located at nucleotide positions 4363-4386 of SEQ ID NO: 115. The loop14 region may have a nucleic acid sequence of SEQ ID NO: 158, or may be a variant thereof having at least 85% (e.g., at least 86%, 90%, 95%, 96%, 97%, 98%, 99%, or more) sequence identity to the nucleic acid sequence of SEQ ID NO: 158, as is shown in Table 4. Accordingly, the disclosed ASO agents may bind within at least a portion of loop 14 region of a Grik2 mRNA.

Alternatively, the disclosed ASO agent may be one that binds to an unpaired region within the secondary structure of the Grik2 mRNA, such as, e.g., an unpaired region 1 located at nucleotide positions 2209-2287 of SEQ ID NO: 115 or positions 1916-1994 of SEQ ID NO: 116. The unpaired region 1 may have a nucleic acid sequence of SEQ ID NO: 159, or may be a variant thereof having at least 85% (e.g., at least 86%, 90%, 95%, 96%, 97%, 98%, 99%, or more) sequence identity to the nucleic acid sequence of SEQ ID NO: 159, as is shown in Table 4. Accordingly, an ASO agent of the disclosure may bind within at least a portion of unpaired region 1 of a Grik2 mRNA. For example, a Grik2 ASO agent that targets a nucleic acid sequence within a portion or region of unpaired region 1 (SEQ ID NO: 159) of SEQ ID NO: 115 or 116 may be siRNA TP (SEQ ID NO: 13), siRNA TO (SEQ ID NO: 14), siRNA MR (SEQ ID NO: 72), or siRNA MQ (SEQ ID NO: 73) or a variant thereof having at least 85% (e.g., at least 86%, 90%, 95%, 96%, 97%, 98%, 99%, or more) sequence identity to the nucleic acid sequence of siRNA TP (SEQ ID NO: 13), siRNA TO (SEQ ID NO: 14), siRNA MR (SEQ ID NO: 72), or siRNA MQ (SEQ ID NO: 73).

In a further example, the ASO agent may be one that binds to an unpaired region 2 located at nucleotide positions 2355-2391 of SEQ ID NO: 115 or positions 2062-2098 of SEQ ID NO: 116. The unpaired region 2 may have a nucleic acid sequence of SEQ ID NO: 160, or may be a variant thereof having at least 85% (e.g., at least 86%, 90%, 95%, 96%, 97%, 98%, 99%, or more) sequence identity to the nucleic acid sequence of SEQ ID NO: 160, as is shown in Table 4. Accordingly, the disclosed ASO agents may bind within at least a portion of unpaired region 2 of a Grik2 mRNA.

As an additional example, the ASO agent may be one that binds to an unpaired region 3 located at nucleotide positions 3324-3368 of SEQ ID NO: 115. The unpaired region 3 may have a nucleic acid sequence of SEQ ID NO: 161, or may be a variant thereof having at least 85% (e.g., at least 86%, 90%, 95%, 96%, 97%, 98%, 99%, or more) sequence identity to the nucleic acid sequence of SEQ ID NO: 161, as is shown in Table 4. Accordingly, an ASO agent of the disclosure may bind within at least a portion of unpaired region 3 of a Grik2 mRNA. For example, a Grik2 ASO agent that targets a nucleic acid sequence within a portion or region of unpaired region 3 (SEQ ID NO: 161) of SEQ ID NO: 115 or 116 may be siRNA G(SEQ ID NO: 15) or a variant thereof having at least 85% (e.g., at least 86%, 90%, 95%, 96%, 97%, 98%, 99%, or more) sequence identity to the nucleic acid sequence of siRNA G(SEQ ID NO: 15).

An ASO agent of the disclosure may also be one that binds to an unpaired region 4 located at nucleotide positions 3587-3639 of SEQ ID NO: 115. The unpaired region 4 may have a nucleic acid sequence of SEQ ID NO: 162, or may be a variant thereof having at least 85% (e.g., at least 86%, 90%, 95%, 96%, 97%, 98%, 99%, or more) sequence identity to the nucleic acid sequence of SEQ ID NO: 162, as is shown in Table 4. The ASO agent may bind within at least a portion of unpaired region 4 of a Grik2 mRNA. For example, a Grik2 ASO agent that targets a nucleic acid sequence within a portion or region of unpaired region 4 (SEQ ID NO: 162) of SEQ ID NO: 115 or 116 may be siRNA TN (SEQ ID NO: 16) or a variant thereof having at least 85% (e.g., at least 86%, 90%, 95%, 96%, 97%, 98%, 99%, or more) sequence identity to the nucleic acid sequence of siRNA TN (SEQ ID NO: 16).

Furthermore, the ASO agent may be one that binds to an unpaired region located at nucleotide positions 3686-3713 of SEQ ID NO: 115. The unpaired region may have a nucleic acid sequence of SEQ ID NO: 163, or may be a variant thereof having at least 85% (e.g., at least 86%, 90%, 95%, 96%, 97%, 98%, 99%, or more) sequence identity to the nucleic acid sequence of SEQ ID NO: 163, as is shown in Table 4. The ASO agent may bind within at least a portion of unpaired region of a Grik2 mRNA. For example, a Grik2 ASO agent that targets a nucleic acid sequence within a portion or region of unpaired region (SEQ ID NO: 163) of SEQ ID NO: 115 or 116 may be siRNA TM (SEQ ID NO: 17) or a variant thereof having at least 85% (e.g., at least 86%, 90%, 95%, 96%, 97%, 98%, 99%, or more) sequence identity to the nucleic acid sequence of siRNA TM (SEQ ID NO: 17).

TABLE 4
cDNA sequences encoding target Grik2mRNA sequences

| | | | SEQ |
|---|---|---|---|
| | Organismal | | ID |
| Description | Source | RefSeq | NO |
| Grik2 mRNA, variant 1; | Homo sapiens | NM_021956.1:4592 | 115 |
| Includes a 5′ UTR and a 3′ UTR | | | |
| Grik2 mRNA, variant 1; (CDS only) | Homo sapiens | NM_021956.294- | 116 |
| Corresponds to nt positions 294-3020 | | 3020 | |
| of SEQ ID NO: 116. | | | |
| Grik2 mRNA, variant 2 | Homo sapiens | NM_175768.3:294- | 117 |
| | | 2903 | |
| Grik2 mRNA, variant 3 | Homo sapiens | NM_001166247.1:2 | 118 |
| | | 94-2972 | |
| Grik2 mRNA, variant 4 | Mus musculus | NM_001111268 | 119 |
| Grik2 mRNA, variant 5 | Mus musculus | NM_010349 | 120 |
| Grik2 mRNA, variant 6 | Mus musculus | NM_001358866 | 121 |
| Grik2 mRNA, variant 7 | Macaca mulatta | XM_015136995.2 | 122 |
| Grik2 mRNA, variant 8 | Macaca mulatta | XM_015136997.2 | 123 |
| Grik2 mRNA, variant 9 | Rattus norvegicus | NM_019309.2 | 124 |
| Grik2 mRNA encoding mature GluK2 peptide | Homo sapiens | NM_021956.1:4592 | 125 |
| CDS | | | |
| Grik2mRNA, 5′ UTR | Homo sapiens | NM_021956.1:4592 | 126 |
| Grik2mRNA, 3′ UTR | Homo sapiens | NM_021956.1:4592 | 127 |
| Grik2mRNA, signal peptide sequence | Homo sapiens | NM_021956.1:4592 | 128 |
| Grik2mRNA, exon 1; | Homo sapiens | NM_021956.1:4592 | 129 |
| nt 1-408 of SEQ ID NO: 115 | | | |
| Grik2mRNA, exon 2; | Homo sapiens | NM_021956.1:4592 | 130 |
| nt 409-576 of SEQ ID NO: 115 | | | |
| Grik2mRNA, exon 3; | Homo sapiens | NM_021956.1:4592 | 131 |
| nt 577-834 of SEQ ID NO: 115 | | | |
| Grik2mRNA, exon 4; | Homo sapiens | NM_021956.1:4592 | 132 |
| nt 835-1016 of SEQ ID NO: 115 | | | |
| Grik2mRNA, exon 5; | Homo sapiens | NM_021956.1:4592 | 133 |
| nt 1017-1070 of SEQ ID NO: 115 | | | |
| Grik2mRNA, exon 6; | Homo sapiens | NM_021956.1:4592 | 134 |
| nt 1071 -1244 of SEQ ID NO: 115 | | | |
| Grik2mRNA, exon 7; | Homo sapiens | NM_021956.1:4592 | 135 |
| nt 1245-1388 of SEQ ID NO: 115 | | | |
| Grik2mRNA, exon 8; | Homo sapiens | NM_021956.1:4592 | 136 |
| nt 1389-1496 of SEQ ID NO: 115 | | | |
| Grik2mRNA, exon 9; | Homo sapiens | NM_021956.1:4592 | 137 |
| nt 1497-1610 of SEQ ID NO: 115 | | | |
| Grik2mRNA, exon 10; | Homo sapiens | NM_021956.1:4592 | 138 |
| nt 1611-1817 of SEQ ID NO: 115 | | | |
| Grik2mRNA, exon 11; | Homo sapiens | NM_021956.1:4592 | 139 |
| nt 1818-2041 of SEQ ID NO: 115 | | | |
| Grik2mRNA, exon 12; | Homo sapiens | NM_021956.1:4592 | 140 |
| nt 2042-2160 of SEQ ID NO: 115 | | | |
| Grik2mRNA, exon 13; | Homo sapiens | NM_021956.1:4592 | 141 |
| nt 2161-2378 of SEQ ID NO: 115 | | | |
| Grik2mRNA, exon 14; | Homo sapiens | NM_021956.1:4592 | 142 |
| nt 2379-2604 of SEQ ID NO: 115 | | | |
| Grik2mRNA, exon 15; | Homo sapiens | NM_021956.1:4592 | 143 |
| nt 2605-2855 of SEQ ID NO: 115 | | | |
| Grik2mRNA, exon 16; | Homo sapiens | NM_021956.1:4592 | 144 |
| nt 2856-4592 of SEQ ID NO: 115 | | | |
| Grik2mRNA, Loop 1 region; nt 494-524 of SEQ | Homo sapiens | NM_021956.1:4592 | 145 |
| ID NO: 116; nt 201-231 of SEQ ID NO: 116 | | | |
| Grik2mRNA, Loop 2 region; nt 1098-1124 of | Homo sapiens | NM_021956.1:4592 | 146 |
| SEQ ID NO: 116; nt 805-831 of SEQ ID NO: 116 | | | |
| Grik2mRNA, Loop 3 region; nt 1197-1237 of | Homo sapiens | NM_021956.1:4592 | 147 |
| SEQ ID NO: 116; nt 904-944 of SEQ ID NO: 116 | | | |
| Grik2mRNA, Loop 4 region; nt 1543-1569 of | Homo sapiens | NM_021956.1:4592 | 148 |
| SEQ ID NO: 116; nt 1250-1276 of SEQ ID NO: | | | |
| 116 | | | |
| Grik2mRNA, Loop 5 region; nt 1667-1731 of | Homo sapiens | NM_021956.1:4592 | 149 |
| SEQ ID NO: 116; nt 1374-1438 of SEQ ID NO: | | | |
| 116 | | | |
| Grik2mRNA, Loop 6 region; nt 1767-1830 of | Homo sapiens | NM_021956.1:4592 | 150 |
| SEQ ID NO: 116; nt 1474-1537 of SEQ ID NO: | | | |
| 116 | | | |
| Grik2mRNA, Loop 7 region; nt 2693-2716 of | Homo sapiens | NM_021956.1:4592 | 151 |
| SEQ ID NO: 116; nt 2400-2423 of SEQ ID NO: | | | |
| 116 | | | |
| Grik2 mRNA, Loop 8 region; nt 2916-2955 of | Homo sapiens | NM_021956.1:4592 | 152 |
| SEQ ID NO: 116; nt 2623-2662 of SEQ ID NO: | | | |
| 116 | | | |
| Grik2mRNA, Loop 9 region; nt 3065-3091 of | Homo sapiens | NM_021956.1:4592 | 153 |
| SEQ ID NO: 116; within 3′ UTR region | | | |
| Grik2mRNA, Loop 10 region; nt 3141-3163 of | Homo sapiens | NM_021956.1:4592 | 154 |
| SEQ ID NO: 116; within 3′ UTR region | | | |
| Grik2mRNA, Loop 11 region; nt 3382-3413 of | Homo sapiens | NM_021956.1:4592 | 155 |
| SEQ ID NO: 116; within 3′ UTR region | | | |
| Grik2mRNA, Loop 12 region; nt 3788-3856 of | Homo sapiens | NM_021956.1:4592 | 156 |
| SEQ ID NO: 116; within 3′ UTR region | | | |
| Grik2mRNA, Loop 13 region; nt 4550-4592 of | Homo sapiens | NM_021956.1:4592 | 157 |
| SEQ ID NO: 116; within 3′ UTR region | | | |
| Grik2mRNA, Loop 14 region; nt 4363-4386 of | Homo sapiens | NM_021956.1:4592 | 158 |
| SEQ ID NO: 116; within 3′ UTR region | | | |
| Grik2mRNA, Unpaired region 1; nt 2209-2287 of | Homo sapiens | NM_021956.1:4592 | 159 |
| SEQ ID NO: 116; nt 1916-1994 of SEQ ID NO: | | | |
| 116 | | | |
| Grik2mRNA, Unpaired region 2; nt 2355-2391 of | Homo sapiens | NM_021956.1:4592 | 160 |
| SEQ ID NO: 116; nt 2062-2098 of SEQ ID NO: | | | |
| 116 | | | |
| Grik2mRNA, Unpaired region 3; nt 3324-3368 of | Homo sapiens | NM_021956.1:4592 | 161 |
| SEQ ID NO: 116; within 3′ UTR region | | | |
| Grik2mRNA, Unpaired region 4; nt 3587-3639 of | Homo sapiens | NM_021956.1:4592 | 162 |
| SEQ ID NO: 116; within 3′ UTR region | | | |
| Grik2mRNA, Unpaired region 5; nt 3686-3713 of | Homo sapiens | NM_021956.1:4592 | 163 |
| SEQ ID NO: 116; within 3′ UTR region | | | |

The ASO agents of the present disclosure may also bind with full or substantial complementarity to any one of the regions of a Grik2 mRNA (e.g., SEQ ID NO: 115) encoded by the nucleotide sequences selected from SEQ ID NOs: 582-681 (see Table 2) or any one of the regions of a Grik2 mRNA encoded by the nucleotide sequences described in SEQ ID NOs: 164-581. For example, an ASO agent of the disclosure may be one that binds to any one of the regions of a Grik2 mRNA (e.g., SEQ ID NO: 115) selected from SEQ ID NOs: 582-681 or a variant thereof having at least 85% (e.g., at least 86%, 90%, 91%, 92%, 93%, 94%, 95%, 96%, 97%, 98%, 99%, or more) sequence identity to the nucleic acid sequence of any one of SEQ ID NOs: 582-681. In another example, an ASO agent of the disclosure may be one that binds to any one of the regions of a Grik2 mRNA (e.g., SEQ ID NO: 115) selected from SEQ ID NOs: 582-681 or a variant thereof having at least 90% (e.g., at least 90%, 91%, 92%, 93%, 94%, 95%, 96%, 97%, 98%, 99%, or more) sequence identity to the nucleic acid sequence of any one of SEQ ID NOs: 582-681. In another example, an ASO agent of the disclosure may be one that binds to any one of the regions of a Grik2 mRNA (e.g., SEQ ID NO: 115) selected from SEQ ID NOs: 582-681 or a variant thereof having at least 95% (e.g., at least 95%, 96%, 97%, 98%, 99%, or more) sequence identity to the nucleic acid sequence of any one of SEQ ID NOs: 582-681. In another example, an ASO agent of the disclosure may be one that binds to any one of the regions of a Grik2 mRNA (e.g., SEQ ID NO: 115) selected from SEQ ID NOs: 582-681. In another example, an ASO agent of the disclosure may be one that binds to any one of the regions of a Grik2 mRNA (e.g., SEQ ID NO: 115) selected from SEQ ID NOs: 164-581 or a variant thereof having at least 85% (e.g., at least 86%, 90%, 91%, 92%, 93%, 94%, 95%, 96%, 97%, 98%, 99%, or more) sequence identity to the nucleic acid sequence of any one of SEQ ID NOs: 164-581. In another example, an ASO agent of the disclosure may be one that binds to any one of the regions of a Grik2 mRNA (e.g., SEQ ID NO: 115) selected from SEQ ID NOs: 164-581 or a variant thereof having at least 90% (e.g., at least 90%, 91%, 92%, 93%, 94%, 95%, 96%, 97%, 98%, 99%, or more) sequence identity to the nucleic acid sequence of any one of SEQ ID NOs: 164-581. In another example, an ASO agent of the disclosure may be one that binds to any one of the regions of a Grik2 mRNA (e.g., SEQ ID NO: 115) selected from SEQ ID NOs: 164-581 or a variant thereof having at least 95% (e.g., at least 95%, 96%, 97%, 98%, 99%, or more) sequence identity to the nucleic acid sequence of any one of SEQ ID NOs: 164-581. In another example, an ASO agent of the disclosure may be one that binds to any one of the regions of a Grik2 mRNA (e.g., SEQ ID NO: 115) selected from SEQ ID NOs: 164-581.

### Modified Oligonucleotides

The ASO agents disclosed herein may contain naturally-occurring and/or modified nucleotides. The oligonucleotide may be modified, particularly chemically modified, in order to increase the stability and/or therapeutic efficiency in vivo. Modifications that will improve the efficacy of an ASO agent of the disclosure, such as a stabilizing modification and/or a modification that reduces RNase H activation in order to avoid degradation of the targeted transcript are known in the art (see, e.g., Bennett and Swayze, Annu. Rev. Pharmacol. Toxicol. 50:259-293, 2010; and Juliano, Nucleic Acids Res. 19;44(14):6518-48, 2016). In particular, the oligonucleotide used in the context of the disclosure may include modified nucleotides. Chemical modifications may occur at three different sites: (i) at phosphate groups, (ii) on the sugar moiety, and/or (iii) on the entire backbone structure of the oligonucleotide. Typically, chemical modifications include backbone modifications, heterocycle modifications, sugar modifications, and conjugation strategies.

For example the oligonucleotide may be selected from the group consisting of oligodeoxyribonucleotides, oligoribonucleotides, small regulatory RNAs (sRNAs), U7- or U1-mediated ASOs or conjugate products thereof such as peptide-conjugated or nanoparticle-complexed ASOs, chemically modified oligonucleotide by backbone modifications such as morpholinos, phosphorodiamidate morpholino oligomers (Phosphorodiamidate morpholinos, PMO), peptide nucleic acid (PNA), phosphorothioate (PS) oligonucleotides, stereochemically pure phosphorothioate (PS) oligonucleotides, phosphoramidates modified oligonucleotides, thiophosphoramidate-modified oligonucleotides, and methylphosphonate modified oligonucleotides; chemically modified oligonucleotide by heterocycle modifications such as bicycle modified oligonucleotides, Bicyclic Nucleic Acid (BNA), tricycle modified oligonucleotides, tricyclo-DNA-antisense oligonucleotides (ASOs), nucleobase modifications such as 5-methyl substitution on pyrimidine nucleobases, 5-substituted pyrimidine analogues, 2-Thio-thymine modified oligonucleotides, and purine modified oligonucleotides; chemically modified oligonucleotide by sugar modifications such as Locked Nucleic Acid (LNA) oligonucleotides, 2′,4′-Methyleneoxy Bridged Nucleic Acid (BNA), ethylene-bridged nucleic acid (ENA), constrained ethyl (cEt) oligonucleotides, 2′-Modified RNA, 2′- and 4′-modified oligonucleotides such as 2′—O—Me RNA (2′-OMe), 2′-O-Methoxyethyl RNA (MOE), 2′-Fluoro RNA (FRNA), and 4′-Thio-Modified DNA and RNA; chemically modified oligonucleotide by conjugation strategies such as N-acetyl galactosamine (GaINAc) oligonucleotide conjugates such as 5′-GaINAc and 3′-GaINAc ASO conjugates, lipid oligonucleotide conjugates, cell penetrating peptides (CPP) oligonucleotide conjugates, targeted oligonucleotide conjugates, antibody-oligonucleotide conjugates, polymer-oligonucleotide conjugate such as with PEGylation and targeting ligand; and chemical modifications and conjugation strategies described for example in Bennett and Swayze, 2010 (supra); Wan and Seth, J Med Chem. 59(21):9645-9667, 20116); Juliano, 2016 (supra); Lundin et al., Hum Gene Ther. 26(8):475-485, 2015); and Prakash, Chem Biodivers. 8(9):1616-1641, 2011). Indeed, for use in vivo, the oligonucleotide may be stabilized. A “stabilized” oligonucleotide refers to an oligonucleotide that is relatively resistant to in vivo degradation (e.g., via an exo- or endonuclease). Stabilization can be a function of length or secondary structure. In particular, oligonucleotide stabilization can be accomplished via phosphate backbone modifications, phosphodiester modifications, phosphorothioate (PS) backbone modifications, combinations of phosphodiester and phosphorothioate modifications, thiophosphoramidate modifications, 2′ modifications (2′—O—Me, 2′—O—(2-methoxyethyl) (MOE) modifications and 2′-fluoro modifications), methylphosphonate, methylphosphorothioate, phosphorodithioate, p-ethoxy, and combinations thereof.

For example, the oligonucleotide may be employed as phosphorothioate derivatives (replacement of a non-bridging phosphoryl oxygen atom with a sulfur atom), which have increased resistance to nuclease digestion. 2′-methoxyethyl (MOE) modification (such as the modified backbone commercialized by IONIS Pharmaceuticals) is also effective. Additionally or alternatively, the oligonucleotide of the present disclosure may include completely, partially or in combination, modified nucleotides which are derivatives with substitutions at the 2′ position of the sugar, in particular with the following chemical modifications: O-methyl group (2′—O—Me) substitution, 2-methoxyethyl group (2′—O—MOE) substitution, fluoro group (2′-fluoro) substitution, chloro group (2′-CI) substitution, bromo group (2′-Br) substitution, cyanide group (2′-CN) substitution, trifluoromethyl group (2′-CF3) substitution, OCF3 group (2′-OCF3) substitution, OCN group (2′-OCN) substitution, O-alkyl group (2′—O-alkyl) substitution, S-alkyl group (2′—S-alkyl) substitution, N-alkyl group (2′-N-akyl) substitution, S-alkenyl group (2′—O-alkenyl) substitution, S-alkenyl group (2′—S-alkenyl) substitution, N-alkenyl group (2′—N-alkenyl) substitution, SOCH3 group (2′-SOCH3) substitution, SO₂CH3 group (2′—SO2CH3) substitution, ONO₂ group (2^{.}-ONO₂) substitution, NO₂ group (2′—NO₂) substitution, N3 group (2′-N3) substitution and/or NH2 group (2′-NH2) substitution. Additionally or alternatively, the oligonucleotide of the disclosure may include completely or partially modified nucleotides wherein the ribose moiety is used to produce locked nucleic acid (LNA), in which a covalent bridge is formed between the 2′ oxygen and the 4′ carbon of the ribose, fixing it in the 3′-endo configuration. These molecules are extremely stable in biological medium, able to activate RNase H such as when LNA are located to extremities (Gapmer) and form tight hybrids with complementary RNA and DNA.

The oligonucleotide used in the context of the disclosure may include modified nucleotides selected from the group consisting of LNA, 2′-OMe analogs, 2′—O-Met, 2′—O—(2-methoxyethyl) (MOE) oligomers, 2′-phosphorothioate analogs, 2′-fluoro analogs, 2′-CI analogs, 2′-Br analogs, 2′-CN analogs, 2′-CF3 analogs, 2′-OCF3 analogs, 2′-OCN analogs, 2′-0-alkyl analogs, 2′-S-alkyl analogs, 2′-N-alkyl analogs, 2′-0-alkenyl analogs, 2′-S-alkenyl analogs, 2′-N-alkenyl analogs, 2′-SOCH3 analogs, 2′-SO₂CH3 analogs, 2′-ONO₂ analogs, 2′-NO₂ analogs, 2′-N3 analogs, 2′-NH2 analogs, tricyclo (tc)-DNAs, U7 short nuclear (sn) RNAs, tricyclo-DNA-oligoantisense molecules and combinations thereof (U.S. Provisional Patent Application Ser. No. 61/212,384 For: Tricyclo-DNA Antisense Oligonucleotides, Compositions and Methods for the Treatment of Disease, filed Apr. 10, 2009, the complete contents of which is hereby incorporated by reference).

In a particular, the oligonucleotide according to the disclosure may be an LNA oligonucleotide. The term “LNA” (Locked Nucleic Acid) (or “LNA oligonucleotide”) refers to an oligonucleotide containing one or more bicyclic, tricyclic or polycyclic nucleoside analogues also referred to as LNA nucleotides and LNA analogue nucleotides. LNA oligonucleotides, LNA nucleotides and LNA analogue nucleotides are generally described in International Publication No. WO 99/14226 and subsequent applications; International Publication Nos. WO 00/56746, WO 00/56748, WO 00/66604, WO 01/25248, WO 02/28875, WO 02/094250, WO 03/006475; U.S. Patent Nos. 6,043,060, 6268490, 6770748, 6639051, and U.S. Publication Nos. 2002/0125241, 2003/0105309, 2003/0125241, 2002/0147332, 2004/0244840 and 2005/0203042, all of which are incorporated herein by reference. LNA oligonucleotides and LNA analogue oligonucleotides are commercially available from, for example, Proligo LLC, 6200 Lookout Road, Boulder, Colo. 80301 USA.

Other forms of oligonucleotides of the present disclosure are oligonucleotide sequences coupled to small nuclear RNA molecules such as U1 or U7 in combination with a viral transfer method based on, but not limited to, lentivirus or adeno-associated virus (Denti, Mass., et al, 2008; Goyenvalle, A, et al, 2004).

Other forms of oligonucleotides of the present disclosure are peptide nucleic acids (PNA). In peptide nucleic acids, the deoxyribose backbone of oligonucleotides is replaced with a backbone more akin to a peptide than a sugar. Each subunit, or monomer, has a naturally occurring or non-naturally occurring base attached to this backbone. One such backbone is constructed of repeating units of N-(2-aminoethyl)glycine linked through amide bonds. Because of the radical deviation from the deoxyribose backbone, these compounds were named peptide nucleic acids (PNAs) (Dueholm et al., New J. Chem., 1997, 21, 19-31). PNA binds both DNA and RNA to form PNA/DNA or PNA/RNA duplexes. The resulting PNA/DNA or PNA/RNA duplexes are bound with greater affinity than corresponding DNA/DNA, DNA/RNA or RNA/RNA duplexes as determined by Tm's. This high thermal stability might be attributed to the lack of charge repulsion due to the neutral backbone in PNA. The neutral backbone of the PNA also results in the Tm's of PNA/DNA(RNA) duplex being practically independent of the salt concentration. Thus, the PNA/DNA(RNA) duplex interaction offers a further advantage over DNA/DNA, DNA/RNA or RNA/RNA duplex interactions which are highly dependent on ionic strength. Homopyrimidine PNAs have been shown to bind complementary DNA or RNA in an anti-parallel orientation forming (PNA)2/DNA(RNA) triplexes of high thermal stability (see, e.g., Egholm, et al., Science, 1991, 254, 1497; Egholm, et al., J. Am. Chem. Soc., 1992, 114, 1895; Egholm, et al., J. Am. Chem. Soc., 1992, 114, 9677). In addition to increased affinity, PNA has also been shown to bind to DNA or RNA with increased specificity. When a PNA/DNA duplex mismatch is melted relative to the DNA/DNA duplex there is seen an 8 to 20° C. drop in the Tm. This magnitude of a drop in Tm is not seen with the corresponding DNA/DNA duplex with a mismatch present. The binding of a PNA strand to a DNA or RNA strand can occur in one of two orientations. The orientation is said to be anti-parallel when the DNA or RNA strand in a 5′ to 3′ orientation binds to the complementary PNA strand such that the carboxyl end of the PNA is directed towards the 5′ end of the DNA or RNA and amino end of the PNA is directed towards the 3′ end of the DNA or RNA. In the parallel orientation the carboxyl end and amino end of the PNA are just the reverse with respect to the 5′-3′ direction of the DNA or RNA. A further advantage of PNA compared to oligonucleotides is that their polyamide backbones (having appropriate nucleobases or other side chain groups attached thereto) is not recognized by either nucleases or proteases and are not cleaved. As a result, PNAs are resistant to degradation by enzymes unlike nucleic acids and peptides. WO 92/20702 describes a peptide nucleic acid (PNA) compounds which bind complementary DNA and RNA more tightly than the corresponding DNA. PNA have shown strong binding affinity and specificity to complementary DNA (Egholm, M., et al., Chem. Soc., Chem. Commun., 1993, 800; Egholm, M., et.al., Nature, 1993, 365, 566; and Nielsen, P., et.al. Nucl. Acids Res., 1993, 21, 197). Furthermore, PNA's show nuclease resistance and stability in cell-extracts (Demidov, V. V., et al., Biochem. Pharmacol., 1994, 48, 1309-1313). Modifications of PNA include extended backbones (Hyrup, B., et.al. Chem. Soc., Chem. Commun., 1993, 518), extended linkers between the backbone and the nucleobase, reversal of the amida bond (Lagriffoul, P. H., et.al., Biomed. Chem. Lett., 1994, 4, 1081), and the use of a chiral backbone based on alanine (Dueholm, K. L, et.al., BioMed. Chem. Lett., 1994, 4, 1077). Peptide Nucleic Acids are described in U.S. Pat. Nos. 5,539,082 and 5,539,083. Peptide Nucleic Acids are further described in U.S. Pat. No. 5,766,855.

The oligonucleotides of the present disclosure (e.g., ASO agents) may be obtained by conventional methods well known in the art. For example, the oligonucleotide of the disclosure can be synthesized de novo using any of a number of procedures well known in the art. For example, the b-cyanoethyl phosphoramidite method (Beaucage et al., 1981); nucleoside H-phosphonate method (Garegg et al., 1986; Froehler et al., 1986, Garegg et al., 1986, Gaffney et al., 1988). These chemistries can be performed by a variety of automated nucleic acid synthesizers available in the market. These nucleic acids may be referred to as synthetic nucleic acids. Alternatively, oligonucleotide can be produced on a large scale in plasmids (see Sambrook, et al., 1989). Oligonucleotide can be prepared from existing nucleic acid sequences using known techniques, such as those employing restriction enzymes, exonucleases or endonucleases. Oligonucleotide prepared in this manner may be referred to as isolated nucleic acids.

Approaches and modifications for enhancing the delivery and the efficacy of oligonucleotides such as chemical modification of the oligonucleotides, lipid- and polymer-based nanoparticles or nanocarriers, ligand-oligonucleotide conjugates by linking oligonucleotides to targeting agents such as carbohydrates, peptides, antibodies, aptamers, lipids or small molecules and small molecules that improve oligonucleotide delivery are well-known in the art, such as described in Juliano (2016; supra). Lipophilic conjugates and lipid conjugates include fatty acid-oligonucleotide conjugates; sterol-oligonucleotide conjugates and vitamin-oligonucleotide conjugates.

The oligonucleotide of the present disclosure can also be modified by substitution at the 3′ or the 5′ end by a moiety including at least three saturated or unsaturated, particularly saturated, linear or branched, particularly linear, hydrocarbon chains including from 2 to carbon atoms, particularly from 5 to carbon atoms, more particularly from to 18 carbon atoms, as described in WO 2014/195432.

The oligonucleotide of the present disclosure may be modified by substitution at the 3′ or the 5′ end by a moiety including at least one ketal functional group, wherein the ketal carbon of said ketal functional group bears two saturated or unsaturated, particularly saturated, linear or branched, particularly linear, hydrocarbon chains including from 1 to 22 carbon atoms, particularly from 6 to carbon atoms, in particular to 19 carbon atoms, and even more particularly from 12 to 18 carbon atoms as described in WO 2014/195430.

Additionally, the oligonucleotide of the present disclosure may be conjugated to a second molecule. Typically, a second molecule may be selected from the group consisting of aptamers, antibodies, or polypeptides. For example, the oligonucleotide of the present disclosure may be conjugated to a cell-penetrating peptide. Cell penetrating peptides are well known in the art and include for example the TAT peptide (see, e.g., Bechara and Sagan, FEBS Lett. 587(12):1693-1702, 2013).

### Delivery of Oligonucleotide Agents to Mammalian Cells

Oligonucleotides of the disclosure can also be delivered using a variety of membranous molecular assembly delivery methods including polymeric, biodegradable microparticle, or microcapsule delivery devices known in the art. For example, a colloidal dispersion system may be used for targeted delivery an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA and can mediate RNase H-mediated gene silencing. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. No. 4,897,355; U.S. Pat. No. 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 10 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169. These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Liposomes may also be sterically stabilized liposomes, including one or more specialized lipids that result in enhanced circulation lifetimes relative to liposomes lacking such specialized lipids. Examples of sterically stabilized liposomes are those in which part of the vesicle-forming lipid portion of the liposome (A) includes one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes including one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes including (1) sphingomyelin and (2) the ganglioside G_{M1} or a galactocerebroside sulfate ester. U.S. Pat. No. 5,543,152 (Webb et al.) discloses liposomes including sphingomyelin. Liposomes including 1,2-sn-dimyristoylphosphatidylcholine are disclosed in WO 97/13499 (Lim et al).

According to the present disclosure, cationic liposomes may be used as a drug delivery vehicle. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: (i) liposomes obtained from natural phospholipids are biocompatible and biodegradable; (ii) liposomes can incorporate a wide range of water and lipid soluble drugs; and (iii) liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotides (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTINTM Bethesda Research Laboratories, Gaithersburg, Md.) is an effective agent for the delivery of highly anionic nucleic acids into living tissue culture cells that include positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Id.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides, e.g., ASO agents described herein, can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present disclosure are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application No. PCT/US2007/080331, filed Oct. 3, 2007 also describes formulations that are amenable to the present disclosure.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotide for use in the methods of the disclosure can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

### Lipid Nanoparticle-Based Delivery Methods

Oligonucleotides of the disclosure may be fully encapsulated in a lipid formulation, e.g., a lipid nanoparticle (LNP), or another nucleic acid-lipid particle. LNPs are extremely useful for systemic applications, as they exhibit extended circulation lifetimes following intravenous injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

The lipid to drug ratio (mass/mass ratio) (e.g., lipid to oligonucleotide ratio) may be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the disclosure.

Non-limiting examples of cationic lipid include N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyetetrahydro-3aH-cyclopenta[d][1,3]dioxo1-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can include, for example, from about mol % to about 50 mol % or about mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-0-monomethyl PE, 16-0-dimethyl PE, 18-1-trans PE, 1-stearoyl-2-oleoyl-phosphatidyethanolamine (SOPE), cholesterol, or a mixture thereof. The non-cationic lipid can be, for example, from about mol % to about 90 mol %, about mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C₈). The conjugated lipid that prevents aggregation of particles can be, for example, from 0 mol % to about mol % or about 2 mol % of the total lipid present in the particle. The nucleic acid-lipid particle may further include cholesterol at, e.g., about mol % to about 60 mol % or about 50 mol % of the total lipid present in the particle.

### Oligonucleotide Conjugated to Ligands

Oligonucleotides of the disclosure may be chemically linked to one or more ligands, moieties, or conjugates that enhance the activity, cellular distribution, or cellular uptake of the oligonucleotide. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

A ligand may alter the distribution, targeting, or lifetime of an oligonucleotide agent into which it is incorporated and/or provide an enhanced affinity for a selected target, e.g., molecule, cell or cell type, compartment, e.g., a cellular or organ compartment, tissue, organ, or region of the body, as, e.g., compared to a species absent such a ligand.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,03-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the oligonucleotide agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

A ligand attached to an oligonucleotide as described herein may act as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that include a number of phosphorothioate linkages are also known to bind to serum protein, thus short oligonucleotides, e.g., oligonucleotides of about bases, bases, bases, or bases, including multiple of phosphorothioate linkages in the backbone are also amenable to the present disclosure as ligands (e.g. as PK modulating ligands). In addition, aptamers that bind serum components (e.g. serum proteins) are also suitable for use as PK modulating ligands in the methods and compositions described herein.

Ligand-conjugated oligonucleotides of the disclosure may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present disclosure may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides of the present disclosure, such as the ligand-molecule bearing sequence-specific linked nucleosides of the present disclosure, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. The oligonucleotides or linked nucleosides of the present disclosure may be synthesized by an automated synthesizer using phosphoramidites derived from ligand-nucleoside conjugates in addition to the standard phosphoramidites and non-standard phosphoramidites that are commercially available and routinely used in oligonucleotide synthesis.

i. Lipid Conjugates

According to the present disclosure, a ligand or conjugate may be a lipid or lipid-based molecule. Such a lipid or lipid-based molecule specifically binds to a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid-based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In another aspect, the ligand may be a moiety, e.g., a vitamin, which is taken up by a target cell, e.g., a proliferating cell. Exemplary vitamins include vitamin A, E, and K.

ii. Cell Permeation Agents

The ligand may also be a cell-permeation agent, for example, a helical cell-permeation agent. In a particular example, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent may be an alpha-helical agent, which has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP containing a hydrophobic MTS can also be a targeting moiety). The peptide moiety can be a “delivery” peptide, which can carry large polar molecules including peptides, oligonucleotides, and protein across cell membranes. For example, sequences from the HIV Tat protein (GRKKRRQRRRPPQ) and the Drosophila Antennapedia protein (RQIKIWFQNRRMKWKK) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to an oligonucleotide agent via an incorporated monomer unit for cell targeting purposes is an arginine-glycine-aspartic acid (RGD)-peptide, or RGD mimic. A peptide moiety can range in length from about amino acids to about amino acids. The peptide moieties can have a structural modification, such as to increase stability or direct conformational properties. Any of the structural modifications described below can be utilized.

An RGD peptide may be used in the compositions of the disclosure for directing the compositions to cellular targets. The RGD peptide may be linear or cyclic, and may be modified, e.g., glycosylated or methylated, to facilitate targeting to a specific tissue(s). RGD-containing peptides and peptidomimetics may include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an a-helical linear peptide (e.g., LL-37 or Ceropin P1), a disulfide bond-containing peptide (e.g., a-defensin, 6-defensin, or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SVlarge T antigen (Simeoni et al., Nucl. Acids Res. 31:2717-2724, 2003).

iii. Carbohydrate Conjugates

According to the compositions and methods of the disclosure, an oligonucleotide may further include a carbohydrate. The carbohydrate conjugated oligonucleotide is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C and above (e.g., C5, C6, C7, or C8) sugars; di- and trisaccharides include sugars having two or three monosaccharide units (e.g., C5, C6, C7, or C8).

In a particular example, a carbohydrate conjugate for use in the compositions and methods of the disclosure is a monosaccharide. The carbohydrate conjugate may further include one or more additional ligands as described above, such as, but not limited to, a PK modulator and/or a cell permeation peptide. Additional carbohydrate conjugates (and linkers) suitable for use in the present disclosure include those described in PCT Publication Nos. WO 2014/179620 and WO 2014/179627, the entire contents of each of which are incorporated herein by reference.

iv. Linkers

The conjugate or ligand described herein can be attached to an oligonucleotide with various linkers that can be cleavable or non-cleavable.

Linkers typically include a direct bond or an atom such as oxygen or sulfur, a unit such as NRB, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenyl heterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In particular examples, the linker may be between about 1-24 atoms, 2-24, 3-24, 4-24, 5-24, 6-24, 6-18, 7-18, 8-18 atoms, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about times, times, times, times, 50 times, 60 times, 70 times, 80 times, 90 times, or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissues. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In some cases, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

a. Redox Cleavable Linking Groups

A cleavable linking group may be a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular oligonucleotide moiety and particular targeting agent one can look to methods described herein. For example, a candidate can be evaluated by incubation with dithioerythritol (DTE), or other reducing agent using reagents know in the art, which mimic the rate of cleavage which would be observed in a cell, e.g., a target cell. The candidates can also be evaluated under conditions which are selected to mimic blood or serum conditions. The candidate compounds may be cleaved by at most about 10% in the blood. In other examples, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

b. Phosphate-Based Cleavable Linking Groups

A cleavable linker may also include a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells.

Examples of phosphate-based linking groups are —O—P(O)(OR^{k})—O—, —O—P(S)(OR^{k})—O—, —O—P(S)(SR^{k})—O—, —S—P(O)(OR^{k})—O—, —O—P(O)(OR^{k})—S—, —S—P(O)(OR^{k})—S—, —O—P(S)(OR^{k})—S—, —S—P(S)(OR^{k})—O—, —O—P(O)(R^{k})—O—, —O—P(S)(R^{k})—O—, —S—P(O)(R^{k})—O—, —S—P(S)(R^{k})—O—,

—S—P(O)(R^{k})—S—, —O—P(S)(R^{k})—S—. These candidates can be evaluated using methods analogous to those described above.

c. Acid Cleavable Linking Groups

A cleavable linker may also include an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula—C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

d. Ester-Based Linking Groups

A cleavable linker may include an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

a Peptide-Based Cleaving Groups

A cleavable linker may further include a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

An oligonucleotide of the disclosure may be conjugated to a carbohydrate through a linker. Linkers include bivalent and trivalent branched linker groups. Exemplary oligonucleotide carbohydrate conjugates with linkers of the compositions and methods of the disclosure include, but are not limited to, those described in formulas 24-of PCT Publication No. WO 2018/195165.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

In certain instances, the nucleotides of an oligonucleotide can be modified by a non-ligand group. A number of non-ligand molecules have been conjugated to oligonucleotides in order to enhance the activity, cellular distribution, or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an amino linker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### Nucleic Acid Vectors

Effective intracellular concentrations of a nucleic acid agent disclosed herein can be achieved via the stable expression of a polynucleotide encoding the agent (e.g., by integration into the nuclear or mitochondrial genome of a mammalian cell). The nucleic acid is an inhibitory RNAs (e.g., ASO agents disclosed herein) targeting the Grik2 mRNA. In order to introduce such exogenous nucleic acids into a mammalian cell, the polynucleotide sequence for the agent can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

The agents disclosed herein can also be introduced into a mammalian cell by targeting a vector containing a polynucleotide encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such, a construct can be produced using conventional and routine methods of the art.

In addition to achieving high rates of transcription and translation, stable expression of an exogenous polynucleotide in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that encodes a Grik2-targeting ASO agent as well as, e.g., additional sequence elements used for the expression of these agents and/or the integration of these polynucleotide sequences into the genome of a mammalian cell. Certain vectors that can be used include plasmids that contain regulatory sequences, such as promoter and enhancer regions, which direct gene transcription. Other useful vectors contain polynucleotide sequences that enhance the rate of translation of these genes or improve the stability or nuclear export of the mRNA that results from gene transcription. These sequence elements include, e.g., 5′ and 3′ UTR regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

### Regulatory Sequences

The ASO agents disclosed herein may be required to be expressed at sufficiently high levels to elicit a therapeutic benefit. Accordingly, polynucleotide expression may be mediated by a promoter sequence capable of driving robust expression of the disclosed ASO agents. According to the methods and compositions disclosed herein, the promoter may be a heterologous promoter. The term “heterologous promoter”, as used herein, refers to a promoter that is not found to be operatively linked to a given encoding sequence in nature. Useful heterologous control sequences generally include those derived from sequences encoding mammalian or viral genes.

For purposes of the present disclosure, both heterologous promoters and other control elements, such as CNS-specific and inducible promoters, enhancers, and the like will be of particular use. A promoter may be derived in its entirety from a native gene (e.g., a Grik2 gene) or may be composed of different elements derived from different naturally-occurring promoters. Alternatively, the promoter may include a synthetic polynucleotide sequence. Different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g. tetracycline-responsive promoters) are well known in the art.

In mammalian systems, three kinds of promoters exist and are candidates for construction of the expression vectors: (i) Pol I promoters that control transcription of large ribosomal RNAs; (ii) Pol II promoters that control the transcription of mRNAs (that are translated into protein), small nuclear RNAs (snRNAs), and endogenous microRNAs (e.g., from introns of pre-mRNA); (iii) and Pol III promoters that uniquely transcribe small non-coding RNAs. Each has advantages and constraints to consider when designing the construct for expression of the RNAs in vivo. For example, Pol III promoters are useful for synthesizing ASO agents (e.g., siRNA, shRNA, miRNA, or shmiRNA, or shmiRNA) from a DNA template in vivo. For greater control over tissue specific expression, Pol II promoters are preferred but can only be used for transcription of miRNAs. When a Pol II promoter is used, however, it may be preferred to omit translation initiation signals so that the RNAs function as siRNA, shRNA or miRNAs and are not translated into peptides in vivo.

Polynucleotides suitable for use with the compositions and methods described herein also include those that encode an ASO agent targeting Grik2 mRNA under control of a mammalian regulatory sequence, such as, e.g., a promoter sequence and, optionally, an enhancer sequence. Exemplary promoters that are useful for the expression of the disclosed ASO agents in mammalian cells include ubiquitous promoters such as, e.g., an H1 promoter, 7SK promoter, apolipoprotein E-human-alpha 1-antitrypsin promoter, CK8 promoter, murine U1 promoter (mU1a), elongation factor 1a (EF-1a) promoter, thyroxine binding globulin (TBG) promoter, phophoglycerate kinase (PKG) promoter, CAG (composite of the (CMV) cytomegalovirus enhancer the chicken beta actin promoter (CBA) and the rabbit beta globin intron), the SVearly promoter, murine mammary tumor virus LTR promoter; adenovirus major late promoter (Ad MLP); a herpes simplex virus (HSV) promoter, a CMV promoter such as the CMV immediate early promoter region (CMV-IE), rous sarcoma virus (RSV) promoter, and U6 promoter or variants thereof. For the purpose of driving cell-type specific expression of inhibitory RNA sequences disclosed herein, cell-type specific promoters may be used. For example, neuron-specific expression of Grik2 ASO agents can be conferred using neuronal-specific promoters, such as, e.g., a human synapsin 1 (hSyn) promoter, hexaribonucleotide binding protein-3 (NeuN) promoter, Ca²⁺/calmodulin-dependent protein kinase II (CaMKII) promoter, tubulin alpha I (Tα-1) promoter, neuron-specific enolase (NSE) promoter, platelet-derived growth factor beta chain (PDGFβ) promoter, vesicular glutamate transporter (VGLUT) promoter, somatostatin (SST) promoter, neuropeptide Y (NPY) promoter, vasoactive intestinal peptide (VIP) promoter, parvalbumin (PV) promoter, glutamate decarboxylase (GAD65 or GAD67) promoter, promoter of Dopamine-1 receptor (DRD1) and Dopamine-2 receptor (DRD2), microtubule-associated protein 1B (MAP1 B), complement component 1 q subcomponent-like 2 (C1q12) promoter, pro-opiomelanocortin (POMC) promoter, and prospero homeobox protein 1 (PROX1) promoter. Variants of the hSyn and CaMKII promoters have been previously described in Hioki et al. Gene Therapy 14:872-82 (2007) and Sauerwald et al. J. Biol. Chem. 265(25):14932-7 (1990), the disclosures of which are hereby incorporated by reference as they relate to specific hSyn and CaMKII promoter sequences. Promoters suitable for driving polynucleotide expression specifically in DG cells of the hippocampus include the C1ql2, POMC, and PROX1 promoters.

In a particular example, the expression vectors of the disclosure include a SYN promoter (e.g., such as a human SYN promoter (hSyn), e.g., any one of SEQ ID NOs: 682-685 and 790 or a variant thereof having at least 85% (e.g., at least 86%, 90%, 95%, 96%, 97%, 98%, 99%, or more) sequence identity to the nucleic acid sequence of any one of SEQ ID NOs: 682-682 and 790). In another example, the expression vectors of the disclosure include a CAMKII promoter (e.g., any one of SEQ ID NOs: 687-691 and 802 or a variant thereof having at least 85% (e.g., at least 86%, 90%, 95%, 96%, 97%, 98%, 99%, or more) sequence identity to the nucleic acid sequence of any one of SEQ ID NOs: 687-691 and 802). In yet another example, the expression vectors of the disclosure include a C1QL2 promoter (e.g., SEQ ID NO: 719 or SEQ ID NO: 791 or a variant thereof having at least 85% (e.g., at least 86%, 90%, 95%, 96%, 97%, 98%, 99%, or more) sequence identity to the nucleic acid sequence of SEQ ID NO: 719 or SEQ ID NO: 791).

Synthetic promoters, hybrid promoters, and the like may also be used in conjunction with the methods and compositions disclosed herein. In addition, sequences derived from non-viral genes, such as the murine metallothionein gene, will also find use herein. Such promoter sequences are commercially available from, e.g., Stratagene (San Diego, Calif.). Exemplary promoter sequences suitable for use with the expression vectors (e.g., plasmid or viral vector, such as, e.g., an AAV or a lentiviral vector) are provided in Table and Table 6 below.

TABLE 5
Exemplary neuron-specific promoter sequences

| | SEQ | | GenBank |
|---|---|---|---|
| Promoter | ID NO | Nucleotide sequence | RefSeq |
| Syn | 682 | CTGCAGAGGGCCCTGCGTATGAGTGCAAGTGG | M55301.1 |
| (short_1; H. | | GTTTTAGGACCAGGATGAGGCGGGGTGGGGGT | |
| sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCGCCGGCGACTCAGCGCTGCC | |
| | | TCAGTCTGC | |
| Syn | 683 | CTGCAGAGGGCCCTGCGTATGAGTGCAAGTGG | M55301.1 |
| (short_2; H. | | GTTTTAGGACCAGGATGAGGCGGGGTGGGGGT | |
| sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCGCCGGCGACTCAGCGCTGCC | |
| | | TCAGTCTGCCAATTGCAGCGGAGGAGTCGTGTC | |
| | | GTGCCTGAGAGCGCAG | |
| Syn | 790 | CTGCAGAGGGCCCTGCGTATGAGTGCAAGTGG | M55301.1 |
| (short_2.5; H. | | GTTTTAGGACCAGGATGAGGCGGGGTGGGGGT | |
| sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCGCCGGCGACTCAGCGCTGCC | |
| | | TCAGTCTGCCAATTGCAGCGGAGGAGTCGTGTC | |
| | | GTGCCTGAGAGCGCAGGGCGCGCC | |
| Syn | 684 | CTGCAGAGGGCCCTGCGTATGAGTGCAAGTGG | M55301.1 |
| (short_3; H. | | GTTTTAGGACCAGGATGAGGCGGGGTGGGGGT | |
| sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCG | |
| Syn | 685 | CTGCAGAGGGCCCTGCGTATGAGTGCAAGTGG | M55301.1 |
| (long_1; H. | | GTTTTAGGACCAGGATGAGGCGGGGTGGGGGT | |
| sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCGCCGGCGACTCAGCGCTGCC | |
| | | TCAGTCTGCGGTGGGCAGCGGAGGAGTCGTGT | |
| | | CGTGCCTGAGAGCGCAGGGCGCGCC | |
| NeuN | 686 | GAGGAGGAGGAGAGAGACCGGGAGGGCGCCC | NG_053112.1 |
| (H. sapiens) | | GGGAGGCAGGGCGCGCGCACACTCCGAGG | |
| CaMKII_1 | 802 | CATTATGGCCTTAGGTCACTTCATCTCCATGGGG | AJ222796.1 |
| | | TTCTTCTTCTGATTTTCTAGAAAATGAGATGGGG | |
| | | GTGCAGAGAGCTTCCTCAGTGACCTGCCCAGGG | |
| | | TCACATCAGAAATGTCAGAGCTAGAACTTGAACT | |
| | | CAGATTACTAATCTTAAATTCCATGCCTTGGGGG | |
| | | CATGCAAGTACGATATACAGAAGGAGTGAACTCA | |
| | | TTAGGGCAGATGACCAATGAGTTTAGGAAAGAA | |
| | | GAGTCCAGGGCAGGGTACATCTACACCACCCGC | |
| | | CCAGCCCTGGGTGAGTCCAGCCACGTTCACCTC | |
| | | ATTATAGTTGCCTCTCTCCAGTCCTACCTTGACG | |
| | | GGAAGCACAAGCAGAAACTGGGACAGGAGCCC | |
| | | CAGGAGACCAAATCTTCATGGTCCCTCTGGGAG | |
| | | GATGGGTGGGGAGAGCTGTGGCAGAGGCCTCA | |
| | | GGAGGGGCCCTGCTGCTCAGTGGTGACAGATA | |
| | | GGGGTGAGAAAGCAGACAGAGTCATTCCGTCAG | |
| | | CATTCTGGGTCTGTTTGGTACTTCTTCTCACGCT | |
| | | AAGGTGGCGGTGTGATATGCACAATGGCTAAAA | |
| | | AGCAGGGAGAGCTGGAAAGAAACAAGGACAGA | |
| | | GACAGAGGCCAAGTCAACCAGACCAATTCCCAG | |
| | | AGGAAGCAAAGAAACCATTACAGAGACTACAAG | |
| | | GGGGAAGGGAAGGAGAGATGAATTAGCTTCCCC | |
| | | TGTAAACCTTAGAACCCAGCTGTTGCCAGGGCA | |
| | | ACGGGGCAATACCTGTCTCTTCAGAGGAGATGA | |
| | | AGTTGCCAGGGTAACTACATCCTGTCTTTCTCAA | |
| | | GGACCATCCCAGAATGTGGCACCCACTAGCCGT | |
| | | TACCATAGCAACTGCCTCTTTGCCCCACTTAATC | |
| | | CCATCCCGTCTGTTAAAAGGGCCCTATAGTTGGA | |
| | | GGTGGGGGAGGTAGGAAGAGCGATGATCACTT | |
| | | GTGGACTAAGTTTGTTCGCATCCCCTTCTCCAAC | |
| | | CCCCTCAGTACATCACCCTGGGGGAACAGGGTC | |
| | | CACTTGCTCCTGGGCCCACACAGTCCTGCAGTA | |
| | | TTGTGTATATAAGGCCAGGGCAAAGAGGAGCAG | |
| | | GTTTTAAAGTGAAAGGCAGGCAGGTGTTGGGGA | |
| | | GGCAGTTACCGGGGCAACGGGAACAGGGCGTT | |
| | | TCGGAGGTGGTTGCCATGGGGACCTGGATGCTG | |
| | | ACGAAGGCTCGCGAGGCTGTGAGCAGCCACAG | |
| | | TGCCCTGCTCAGAAGCCCCAAGCTCGTCAGTCA | |
| | | AGCCGGTTCTCCGTTTGCACTCAGGAGCACGGG | |
| | | CAGGCGAGTGGCCCCTAGTTCTGGGGGCAGC | |
| CaMKII | 687 | GATGCTGACGAAGGCTCGCGAGGCTGTGAGCA | NM_171825 |
| (α; H. sapiens) | | GCCACAGTGCCCTGCTCAGAAGCCCCGG | |
| CaMKII | 688 | GTCTCCCGCGCCCGCGCCCGTGTCGCCGCCGT | NM_172084 |
| (β1; H. sapiens) | | GCCCGCGAGCGGGAGCCGGAGTCGCCGC | |
| CaMKII | 689 | CGTGTGCAGATGCAGGGCGCCGGTGCCCTGCG | NM_172084 |
| (β2; H. sapiens) | | GGTGCGGGTGCAGGAGCAGCGTGTGCAG | |
| CaMKII | 690 | CCCCACGCCACCCTTTCTGGTCATCTCCCCTCC | NM_172115 |
| (δ; H. sapiens) | | CGCCCCGCCCCTGCGCACACTCCCTCG | |
| CaMKII | 691 | TCTCCCCGGTAAAGTCTCGCGGTGCTGCCGGGC | NM_172171 |
| (γ; H. sapiens) | | TCAGCCCCGTCTCCTCCTCTTGCTCCC | |
| NSE/ENO2 | 692 | CGCCTCCTCCGCCCGCCGCCCGGGAGCCGCAG | NM_001975 |
| (isoform 1; H. | | CCGCCGCCGCCACTGCCACTCCCGCTCT | |
| sapiens) | | | |
| NSE/ENO2 | 693 | TGGGTGCCCCCACCCTTCCCCCATCCTCCTCCC | NM_001975 |
| (isoform 2; H. | | TTCCCCACTCCACCCTCGTCGGTCCCC | |
| sapiens) | | | |
| PDGFβ | 694 | AAAAAAAAAAAAAAAGCCCACCCTCCAGCCTCGC | NM_033016 |
| (isoform 1; H. | | TGCAAAGAGAAAACCGGAGCAGCCGC | |
| sapiens) | | | |
| PDGFβ | 695 | TCTCGCACTCTCCCTTCTCCTTTATAAAGGCCGG | NM_033016 |
| (isoform 2; H. | | AACAGCTGAAAGGGTGGCAACTTCTC | |
| sapiens) | | | |
| PDGFβ | 696 | GCCGCGTCCACCTGTCGGCCGGGCCCAGCCGA | NM_033016 |
| (isoform 3; H. | | GCGCGCAGCGGGCACGCCGCGCGCGCGG | |
| sapiens) | | | |
| VGluT1/SLC17A7 | 697 | GCGCCCCGCCCCCGGCGCTGAGTCCTGTGACA | NM_020309 |
| (H. sapiens) | | GCCCCCGGGCCGCCTGCACTTGCAGCCT | |
| VGluT2/SLC17A6 | 698 | AAAGAAGAGTCCCCTATTCCTGAAACTTACTCTG | NM_020346 |
| (isoform 1; H. | | TCCGTGGTGCTGAAACATTGTACCGA | |
| sapiens) | | | |
| VGluT2/SLC17A6 | 699 | CGTCCTCAAAGAGCAGCAAGCCTTCTCCATCTTA | NM_020346 |
| (isoform 2; H. | | ATTTGACTCTACCGCAGAGCAGACTT | |
| sapiens) | | | |
| VGluT2/SLC17A6 | 700 | ATGCAGCTATTCTGTTGTATTCTCATTCTCACTCT | NM_020346 |
| (isoform 3; H. | | CCCTCCCTTCTCTCACTCTCACTCT | |
| sapiens) | | | |
| VGluT3/SLC17A8 | 701 | CATGTTAGCGTCCCCAGCTGCAGCCCAGGGAGG | NM_001145288 |
| (H. sapiens) | | GAGAGAGGCTGCGCTCAGTCTGAGAGT | |
| SST | 702 | TGACGTCAGAGAGAGAGTTTAAAACAGAGGGAG | NM_001048 |
| (isoform 1; H. | | ACGGTTGAGAGCACACAAGCCGCTTTA | |
| sapiens) | | | |
| SST | 703 | GAGTGAAAATAAAAGATTGTATAAATCGTGGGGC | NM_001048 |
| (isoform 2; H. | | ATGTGGAATTGTGTGTGCCTGTGCGT | |
| sapiens) | | | |
| NPY | 704 | GCCGCGGCGAGGAAGCTCCATAAAAGCCCTGTC | NM_000905 |
| (H. sapiens) | | GCGACCCGCTCTCTGCACCCCATCCGC | |
| VIP | 705 | CAGTCCTAAGTATAAGCCCTATAAAATGATGGGC | NM_194435 |
| (isoform 1; H. | | TTTGAAATGCTGGTCAGGGTAGAGTG | |
| sapiens) | | | |
| VIP | 706 | TTTTCCATTAATGTTTTCAGACTGCTGTTGACCAC | NM_194435 |
| (isoform 2; H. | | AGGTAACTGAAATCATGGAAAGAGA | |
| sapiens) | | | |
| PV | 707 | TGGTCATATGAGCAGAAATGATGAGAAAAGCACT | NM_002854 |
| (isoform 1; H. | | TTTTAATCTTTTCGCACTTGCTCTGC | |
| sapiens) | | | |
| PV | 708 | AATAGCCAGAGCAGAAGCCTATATAGGTGGCCA | NM_002854 |
| (isoform 2; H. | | TCCCACCTCCAGGCTCACTTCCCGACA | |
| sapiens) | | | |
| PV | 709 | CAGCGCTCAGATTTTGCAGCATAAATTTGCATCC | NM_002854 |
| (isoform 3; H. | | AGGACAGACCAGAGCAGAGGCTGAGG | |
| sapiens) | | | |
| GAD65/GAD2 | 710 | GCACGCACGCGCGCGCAGGGCCAAGCCCGAGG | NM_001134366 |
| (isoform 1; H. | | CAGCTCGCCCGCAGCTCGCACTCGCAGG | |
| sapiens) | | | |
| GAD65/GAD2 | 711 | CCCGCCTCTGGCTCGCCCGAGGACGCGCTGGC | NM_001134366 |
| (isoform 2 H. | | ACGCCTCCCACCCCCTCACTCTGACTCC | |
| sapiens) | | | |
| GAD65/GAD2 | 712 | CACTGGGCTCCCTTTCCCTCAAATGCTCTGGGG | NM_001134366 |
| (isoform 3; H. | | CTCTCCGCGCTTTCCTGAGTCCGGGCT | |
| sapiens) | | | |
| GAD65/GAD2 | 713 | CACAGAAAACTCCTCTGGGCCACGCTTCCCGCC | NM_001134366 |
| (isoform 4; H. | | TCGCCGAGGTCTCCCCAGTCTGCCCCT | |
| sapiens) | | | |
| GAD67/GAD1 | 714 | CTCTGCCCCCGCCTACCCCGGAGCCGTGCAGC | NM_013445 |
| (isoform 1 H. | | CGCCTCTCCGAATCTCTCTCTTCTCCTG | |
| sapiens) | | | |
| GAD67/GAD1 | 715 | CTGGATTTATAATCGCCCTATAAAGCTCCAGAGG | NM_013445 |
| (isoform 2; H. | | CGGTCAGGCACCTGCAGAGGAGCCCC | |
| sapiens) | | | |
| DRD1 | 716 | GGGACGCGCGGGCGGGGTGGGCTGTGCCCCG | NM_000794 |
| (H. sapiens) | | CGGGAACCCCGCCGGCCTGTGCGCTTGCTG | |
| DRD2 | 717 | CTCCCTCCCGCGCTCCCCGCGCTCGGGCGCCG | NM_016574 |
| (isoform 1; H. | | CAGAGCTGTCCAGCTTCAGTGCCGAACC | |
| sapiens) | | | |
| DRD2 | 718 | GTACTGGTGTACAAGGACAAGGTGACTTTTTTTC | NM_016574 |
| (isoform 2; H. | | TTTTCCCAGATTGAAAGGGCCAAAGA | |
| sapiens) | | | |
| C1ql2 1 | 719 | CCTCCGCCGCTCAGCCCCGGACTCCTTACGTCA | NM_182528 |
| (H. sapiens) | | GGGTAGCGGGGTCCCCCCTCCGCGCGG | |
| C1ql2 2 | 791 | CGATCCTATCACGAGACTAGCCTCGAGAAGCTT | AC01667.5 |
| (H. sapiens) | | GATATCAGCACCCACATAGCAGCTCACAAATGTC | AC084310 |
| | | TGAAACTCCAATTCTTGGGAATCTGACACGATCA | |
| | | CACATGCAGGCAAAATACCAATGTACATGAATTA | |
| | | AAAAAAAAAAAAACAACCTTTAAAAGAAACAAGG | |
| | | GTTCAGTACCACTACTGACATCTTGTTTCCCCAG | |
| | | AGGCCTTACTTTAATTATTTATTGTTTCCACTTAG | |
| | | TTGCTCAATTAATTAATTTAGAGGTTTTTTTCTTC | |
| | | CTTTCTTTTTCTTTTTTCTTTCTCTCTTTTTTTTC | |
| | | TTCTTAAGACAGGGTTTCTCTGTGTAGCTCAGGCT | |
| | | ATCCTGGAACTCACTCTGTAGACCAGGCTGGCCT | |
| | | TGTACTCAAAGATCTGCCTGCCTCTGCCTCCCCA | |
| | | GTGCTGGGATTAAAGACATGCACCATCACTGCC | |
| | | CTGCTTTCCTCTTTTTATTTTGAAAATTGTTCATC | |
| | | AACAGTTACTAAACGTGTTCGAATTCCAAGAGCT | |
| | | GACTAGACATATAAGACCATTCAGCCTTCTGAAT | |
| | | AAGATGTAGGTGTGCCCCTCCTCTTACTCCTCTA | |
| | | TTTGGAAGTTGGTTACTTTCTGTATGTAGTATGC | |
| | | GAATCCCCCTCTGCCACCCCGCTTTCTGTTTTAA | |
| | | AACAGAAAAGGCTGCAACATACAGTGTGTGCTTC | |
| | | TGTTCTTGAACTGGAAGCTTAGGCTGTCCTGGAC | |
| | | TTGGGTTGAGACCTGGGCTCATCCAGATAGGAA | |
| | | ATGGATTTGGTGACCCCGCCAGGACTTCGCAGG | |
| | | CACCACATCGTGGTCGTGTGTGGGTGCTGTATG | |
| | | CACCCACTGATTGCGCGCGTGGGTTCCAGAGCT | |
| | | TGGTGGTCTGCGAGAGGAGAGTGGGCAAGAGT | |
| | | GGGTGTGTCTGTGGAGCCCCAGCTAGGGGCTG | |
| | | CTGCCCGCTGCTCCCACTTGTGGCTCCTGGGCG | |
| | | CCGCCAGCAGGCACATCTCCGGAGGACGCCGC | |
| | | GGGATGGGAGCTGATGACAGGAGAGCGCCGTC | |
| | | TCCCGAGTGATGGCAGCGCACGCTGCTGCCTCG | |
| | | CCGCCTCCGCCGCTCAGTCCTGATCTTACGTTA | |
| | | GGGTAGCTGGGTACCCCCTCCGCCCGGGAACC | |
| | | AGCTAGTAGAGGGAGAACAGAGCAGAGCGTGC | |
| | | GGCAGAGCCGATCCCGCGTCCCGCCGAACCCT | |
| | | GCCAAGCCCCGCCAATCCCAGCAGAGCAGGAA | |
| | | CCAGCGCAGCTGAGCCAACACCGGACGCCGCA | |
| | | CTGAGACCCAGCATTCCCCAGCCGCCACTACCC | |
| | | GGTCCCCGCCGGGGTGCCGGGCTCGTCCTGTG | |
| | | AGCCCCTCGTCATGCGTGTCGGGCTCTTCGACT | |
| | | CTCCAGATCAGTTCCAGAGCGCT | |
| POMC | 720 | CCAGGAGAGCTCGGCAAGTATATAAGGACAGAG | NM_001319204 |
| (H. sapiens) | | GAGCGCGGGACCAAGCGGCGGCGAAGG | |
| PROX1 | 721 | TTCCTTCAGCTGTGTCTTAAAGTAAATCTTGTTGT | NM_002763 |
| (isoform 1; H. | | GGAGCGGAGCCCTCAGCTGAGGGAG | |
| sapiens) | | | |
| PROX1 | 722 | GTAAGTATCTTCTTCTTCCCCTCGTGAGTCCCTC | NM_002763 |
| (isoform 2) | | CCCTTTTCCAGAATCACTTGCACTGT | |
| MAP1B | 723 | GGGGCGGAGCGGAGACAGTACCTTCGGAGATA | NM_005909 |
| (isoform 1; H. | | ATCCTTTCTCCTGCCGCAGTGGAGAGGA | |
| sapiens) | | | |
| MAP1B | 724 | CCCTGCCTAGTCTCCATATAAAAGCGGCGCCGC | NM_005909 |
| (isoform 2; H. | | CTCCCCGCCCTCTCTCACTCCCCGCTC | |
| sapiens) | | | |
| MAP1B | 725 | GGGCGGCCCAGCCCCAGGTTACGTCGTCCCCA | NM_005909 |
| (isoform 3; H. | | GAAAGAATCTGGCCAACAGTCTGGCCGT | |
| sapiens) | | | |
| Tα-1/TUBA1A | 726 | ATGCTAATACACCTTAATTTTACGATTTTTTCACT | NM_006009 |
| (isoform 1; H. | | TTTCCTCCCCACAGCGTGAGTGCAT | |
| sapiens) | | | |
| Tα-1/TUBA1A | 727 | TAACCCCAGTCCCCTTTCTTCTCCTTCCGCCCCT | NM_006009 |
| (isoform 1; H. | | CCCCAACCCCGCCCCATAATGGATGC | |
| sapiens) | | | |

In a particular example, a viral vector of the disclosure (e.g., an AAV vector) incorporates a neuron-specific promoter sequence. In a particular example, the neuron-specific promoter is a human Syn promoter, such as, a human Syn promoter having a nucleic acid sequence of any one of SEQ ID NOs: 682-685 and SEQ ID NO: 790 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of any one of SEQ ID NOs: 682-685 and SEQ ID NO: 790.

In another example, the neuron-specific promoter is a NeuN promoter sequence, such as a NeuN promoter sequence of SEQ ID NO: 686 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 686.

In another example, the neuron-specific promoter is a CaMKII promoter sequence, such as a CaMKII promoter sequence of any one of SEQ ID NOs: 687-691 and SEQ ID NO: 802 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of any one of SEQ ID NOs: 687-691 and SEQ ID NO: 802.

Additional CaMKII promoters may include the human alpha CaMKII promoter sequence described in Wang et al. (Mol. Biol. Rep. 35(1): 37-44, 2007), the disclosure of which is incorporated in its entirety herein as it relates to the CaMKII promoter sequence.

In another example, the neuron-specific promoter is a NSE promoter sequence, such as a NSE promoter sequence of SEQ ID NOs: 692 or SEQ ID NO: 693 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 692 or SEQ ID NO: 693.

In another example, the neuron-specific promoter is a PDGFβ promoter sequence, such as a PDGFβ promoter sequence of SEQ ID NOs: 694-696 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 694-696.

In another example, the neuron-specific promoter is a VGIuT promoter sequence, such as a VGIuT promoter sequence of SEQ ID NOs: 697-701or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 708-712.

In another example, the neuron-specific promoter is a SST promoter sequence, such as a SST promoter sequence of SEQ ID NO: 702 or SEQ ID NO: 703 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 702 or SEQ ID NO: 703.

In another example, the neuron-specific promoter is a NPY promoter sequence, such as a NPY promoter sequence of SEQ ID NO: 704 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 704.

In another example, the neuron-specific promoter is a VIP promoter sequence, such as a VIP promoter sequence of SEQ ID NOs: 705 or SEQ ID NO: 706 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 705 or SEQ ID NO: 706.

In another example, the neuron-specific promoter is a PV promoter sequence, such as a PV promoter sequence of SEQ ID NOs: 707-709 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 718-720.

In another example, the neuron-specific promoter is a GAD65 promoter sequence, such as a GAD65 promoter sequence of SEQ ID NOs: 710-713 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 710-713.

In another example, the neuron-specific promoter is a GAD67 promoter sequence, such as a GAD67 promoter sequence of SEQ ID NO: 714 or SEQ ID NO: 715 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 714 or SEQ ID NO: 715.

In another example, the neuron-specific promoter is a DRD1 promoter sequence, such as a DRD1 promoter sequence of SEQ ID NO: 716 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 716.

In another example, the neuron-specific promoter is a DRD2 promoter sequence, such as a DRD2 promoter sequence of SEQ ID NO: 717 or SEQ ID NO: 718 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 717 or SEQ ID NO: 718.

In another example, the neuron-specific promoter is a C1ql2promoter sequence, such as a C1ql2promoter sequence of SEQ ID NO: 719 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 719 or SEQ ID NO: 791.

In another example, the neuron-specific promoter is a POMC promoter sequence, such as a POMC promoter sequence of SEQ ID NO: 720 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 720.

In another example, the neuron-specific promoter is a PROX1 promoter sequence, such as a PROX1 promoter sequence of SEQ ID NO: 721 or SEQ ID NO: 722 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 737 or 738.

In yet another example, the neuron-specific promoter is a MAP1 B promoter sequence, such as a MAP1 B promoter sequence of SEQ ID NOs: 723-725 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 723-725.

In yet another example, the neuron-specific promoter is a Ta-1 promoter sequence, such as a Tα-1 promoter sequence of SEQ ID NO: 726 or SEQ ID NO: 727 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 726 or SEQ ID NO: 727.

TABLE 6
Exemplary ubiquitous promoter sequences

| | SEQ | | |
|---|---|---|---|
| Promoter | ID NO | Nucleotide sequence | RefSeq |
| U6 small nuclear | 728 | CCCCAGTGGAAAGACGCGCAGGCAAAACGCAC | M14486.1 |
| 1; Homo sapiens | | CACGTGACGGAGCGTGACCGCGCGCCGAGCCC | |
| | | AAGGTCGGGCAGGAAGAGGGCCTATTTCCCATG | |
| | | ATTCCTTCATATTTGCATATACGATACAAGGCTGT | |
| | | TAGAGAGATAATTAGAATTAATTTGACTGTAAACA | |
| | | CAAAGATATTAGTACAAAATACGTGACGTAGAAA | |
| | | GTAATAATTTCTTGGGTAGTTTGCAGTTTTAAAAT | |
| | | TATGTTTTAAAATGGACTATCATATGCTTACCGTA | |
| | | ACTTGAAAGTATTTCGATTTCTTGGCTTTATATAT | |
| | | CTTGTGGAAAGGACGAAACACCGTGCTCGCTTC | |
| | | GGCAGCACATATACTAAAATTGGAACGATACAGA | |
| | | GAAGATTAGCATGGCCCCTGCGCAAGGATGACA | |
| | | CGCAAATTCGTGAAGCGTTCCATATTTTTACATC | |
| | | AGGTTGTTTTTCTGTTTTTACATCAGGTTGTTTTT | |
| | | CTGTTTGGTTTTTTTTTTACACCACGTTTATACGC | |
| | | CGGTGCACGGTTTACCA | |
| U6 short | 729 | GAGGGCCTATTTCCCATGATTCCTTCATATTTGC | M14486.1 |
| 249 bp | | ATATACGATACAAGGCTGTTAGAGAGATAATTAG | |
| | | AATTAATTTGACTGTAAACACAAAGATATTAGTAC | |
| | | AAAATACGTGACGTAGAAAGTAATAATTTCTTGG | |
| | | GTAGTTTGCAGTTTTAAAATTATGTTTTAAAATGG | |
| | | ACTATCATATGCTTACCGTAACTTGAAAGTATTTC | |
| | | GATTTCTTGGCTTTATATATCTTGTGGAAAGGAC | |
| | | GAAACACCG | |
| minimal U6 | 730 | GAGGGCCTATTTCCCATGATTCCTTCATATTTGC | M14486.1 |
| 111 bp | | ATATACGATAGCTTACCGTAACTTGAAAGTATTTC | |
| | | GATTTCTTGGCTTTATATATCTTGTGGAAAGGAC | |
| | | GAAACACCG | |
| U6 variant- | 731 | TTTCGATTTCTTGGCTTTATATATCTTGTGGAAAG | (Preece et al, |
| TATA only 46 bp | | GACGAAACACCG | Gene Therapy, |
| | | | 2020) |
| U6 variant- | 732 | ATACGATAGCTTACCGTAACTTGAAAGTATTTCG | (Preece et al, |
| PSE + TATA | | ATTTCTTGGCTTTATATATCTTGTGGAAAGGACG | Gene Therapy, |
| 75 bp | | AAACACCG | 2020) |
| U6 variant-SPH- | 733 | GAGGGCCTATTTCCCATGATTCCTTCATATTTGC | (Preece et al, |
| OCT + TATA 82 bp | | ATTTTCGATTTCTTGGCTTTATATATCTTGTGGAA | Gene Therapy, |
| | | AGGACGAAACG | 2020) |
| U6 (mouse) | 772 | ATCCGACGCCGCCATCTCTAGGCCCGCGCCGG | |
| | | CCCCCTCGCACAGACTTGTGGGAGAAGCTCGGC | |
| | | TACTCCCCTGCCCCGGTTAATTTGCATATAATAT | |
| | | TTCCTAGTAACTATAGAGGCTTAATGTGCGATAA | |
| | | AAGACAGATAATCTGTTCTTTTTAATACTAGCTAC | |
| | | ATTTTACATGATAGGCTTGGATTTCTATAAGAGAT | |
| | | ACAAATACTAAATTATTATTTTAAAAAACAGCACA | |
| | | AAAGGAAACTCACCCTAACTGTAAAGTAATTGTG | |
| | | TGTTTTGAGACTATAAATATCCCTTGGAGAAAAG | |
| | | CCTTGTT | |
| H1 | 734 | AATATTTGCATGTCGCTATGTGTTCTGGGAAATC | X16612.1 |
| | | ACCATAAACGTGAAATGTCTTTGGATTTGGGAAT | |
| | | CTTATAAGTTCTGTATGAGACCACTCTTTCCC | |
| 7SK | 735 | CTGCAGTATTTAGCATGCCCCACCCATCTGCAAG | X05490.1 |
| | | TGCATTCTGGATAGTGTCAAAACAGGCGGAAATC | |
| | | AAGTCCGTTTATCTCAAACTTTAGCATTTTGGGA | |
| | | ATAAATGATATTTGCTATGCTGGTTAAATTAGATT | |
| | | TTAGTTAAATTTCCTGATGAAGCTCTAGTACGATA | |
| | | AGCAACTTGACCTAAGTGTAAAGTTGAGATTTCC | |
| | | TTCAGGTTTATATAGCTTGTGCGCCGCCTGGGTA | |
| | | CCTC | |
| ApoE.hAAT | 736 | AGGCTCAGAGGCACACAGGAGTTTCTGGGCTCA | |
| | | CCCTGCCCCCTTCCAACCCCTCAGTTCCCATCCT | |
| | | CCAGCAGCTGTTTGTGTGCTGCCTCTGAAGTCC | |
| | | ACACTGAACAAACTTCAGCCTACTCATGTCCCTA | |
| | | AAATGGGCAAACATTGCAAGCAGCAAACAGCAA | |
| | | ACACACAGCCCTCCCTGCCTGCTGACCTTGGAG | |
| | | CTGGGGCAGAGGTCAGAGACCTCTCTGGGCCC | |
| | | ATGCCACCTCCAACATCCACTCGACCCCTTGGA | |
| | | ATTTCGGTGGAGAGGAGCAGAGGTTGTCCTGGC | |
| | | GTGGTTTAGGTAGTGTGAGAGGGGTACCCGGG | |
| | | GATCTTGCTACCAGTGGAACAGCCACTAAGGATT | |
| | | CTGCAGTGAGAGCAGAGGGCCAGCTAAGTGGTA | |
| | | CTCTCCCAGAGACTGTCTGACTCACGCCACCCC | |
| | | CTCCACCTTGGACACAGGACGCTGTGGTTTCTG | |
| | | AGCCAGGTACAATGACTCCTTTCGGTAAGTGCA | |
| | | GTGGAAGCTGTACACTGCCCAGGCAAAGCGTCC | |
| | | GGGCAGCGTAGGCGGGCGACTCAGATCCCAGC | |
| | | CAGTGGACTTAGCCCCTGTTTGCTCCTCCGATAA | |
| | | CTGGGGTGACCTTGGTTAATATTCACCAGCAGC | |
| | | CTCCCCCGTTGCCCCTCTGGATCCACTGCTTAAA | |
| | | TACGGACGAGGACAGGGCCCTGTCTCCTCAGCT | |
| | | TCAGGCACCACCACTGACCTGGGACAGT | |
| CAG | 737 | GACATTGATTATTGACTAGTTATTAATAGTAATCA | |
| | | ATTACGGGGTCATTAGTTCATAGCCCATATATGG | |
| | | AGTTCCGCGTTACATAACTTACGGTAAATGGCCC | |
| | | GCCTGGCTGACCGCCCAACGACCCCCGCCCATT | |
| | | GACGTCAATAATGACGTATGTTCCCATAGTAACG | |
| | | CCAATAGGGACTTTCCATTGACGTCAATGGGTG | |
| | | GAGTATTTACGGTAAACTGCCCACTTGGCAGTAC | |
| | | ATCAAGTGTATCATATGCCAAGTACGCCCCCTAT | |
| | | TGACGTCAATGACGGTAAATGGCCCGCCTGGCA | |
| | | TTATGCCCAGTACATGACCTTATGGGACTTTCCT | |
| | | ACTTGGCAGTACATCTACGTATTAGTCATCGCTA | |
| | | TTACCATGGTCGAGGTGAGCCCCACGTTCTGCT | |
| | | TCACTCTCCCCATCTCCCCCCCCTCCCCACCCC | |
| | | CAATTTTGTATTTATTTATTTTTTAATTATTTTGTG | |
| | | CAGCGATGGGGGCGGGGGGGGGGGGGGGGCG | |
| | | CGCGCCAGGCGGGGCGGGGCGGGGCGAGGGG | |
| | | CGGGGCGGGGCGAGGCGGAGAGGTGCGGCGG | |
| | | CAGCCAATCAGAGCGGCGCGCTCCGAAAGTTTC | |
| | | CTTTTATGGCGAGGCGGCGGCGGCGGCGGCCC | |
| | | TATAAAAAGCGAAGCGCGCGGCGGGCGGGAGT | |
| | | CGCTGCGCGCTGCCTTCGCCCCGTGCCCCGCT | |
| | | CCGCCGCCGCCTCGCGCCGCCCGCCCCGGCTC | |
| | | TGACTGACCGCGTTACTCCCACAGGTGAGCGGG | |
| | | CGGGACGGCCCTTCTCCTCCGGGCTGTAATTAG | |
| | | CGCTTGGTTTAATGACGGCTTGTTTCTTTTCTGT | |
| | | GGCTGCGTGAAAGCCTTGAGGGGCTCCGGGAG | |
| | | GGCCCTTTGTGCGGGGGGAGCGGCTCGGGGGG | |
| | | TGCGTGCGTGTGTGTGTGCGTGGGGAGCGCCG | |
| | | CGTGCGGCTCCGCGCTGCCCGGCGGCTGTGAG | |
| | | CGCTGCGGGCGCGGCGCGGGGCTTTGTGCGCT | |
| | | CCGCAGTGTGCGCGAGGGGAGCGCGGCCGGG | |
| | | GGCGGTGCCCCGCGGTGCGGGGGGGGCTGCG | |
| | | AGGGGAACAAAGGCTGCGTGCGGGGTGTGTGC | |
| | | GTGGGGGGGTGAGCAGGGGGTGTGGGCGCGT | |
| | | CGGTCGGGCTGCAACCCCCCCTGCACCCCCCT | |
| | | CCCCGAGTTGCTGAGCACGGCCCGGCTTCGGG | |
| | | TGCGGGGCTCCGTACGGGGCGTGGCGCGGGG | |
| | | CTCGCCGTGCCGGGCGGGGGGTGGCGGCAGG | |
| | | TGGGGGTGCCGGGCGGGGCGGGGCCGCCTCG | |
| | | GGCCGGGGAGGGCTCGGGGGAGGGGCGCGGC | |
| | | GGCCCCCGGAGCGCCGGCGGCTGTCGAGGCG | |
| | | CGGCGAGCCGCAGCCATTGCCTTTTATGGTAAT | |
| | | CGTGCGAGAGGGCGCAGGGACTTCCTTTGTCCC | |
| | | AAATCTGTGCGGAGCCGAAATCTGGGAGGCGCC | |
| | | GCCGCACCCCCTCTAGCGGGCGCGGGGCGAAG | |
| | | CGGTGCGGCGCCGGCAGGAAGGAAATGGGCGG | |
| | | GGAGGGCCTTCGTGCGTCGCCGCGCCGCCGTC | |
| | | CCCTTCTCCCTCTCCAGCCTCGGGGCTGTCCGC | |
| | | GGGGGGACGGCTGCCTTCGGGGGGGACGGGG | |
| | | CAGGGCGGGGTTCGGCTTCTGGCGTGTGACCG | |
| | | GCGGCTCTAGAGCCTCTGCTAACCATGTTCATG | |
| | | CCTTCTTCTTTTTCCTACAGCTCCTGGGCAACGT | |
| | | GCTGGTTATTGTGCTGTCTCATCATTTTGGCAAA | |
| | | G | |
| CB/CBA | 738 | CGCGTGGTACCTCTGGTCGTTACATAACTTACG | |
| | | GTAAATGGCCCGCCTGGCTGACCGCCCAACGAC | |
| | | CCCGCCCATTGACGTCAATAATGACGTATGTTCC | |
| | | CATAGTAACGCCAATAGGGACTTTCCATTGACGT | |
| | | CAATGGGTGGAGTATTTACGGTAAACTGCCCACT | |
| | | TGGCAGTACATCAAGTGTATCATATGCCAAGTAC | |
| | | GCCCCCTATTGACGTCAATGACGGTAAATGGCC | |
| | | CGCCTGGCATTATGCCCAGTACATGACCTTATG | |
| | | GGACTTTCCTACTTGGCAGTACATCTACTCGAGG | |
| | | CCACGTTCTGCTTCACTCTCCCCATCTCCCCCCC | |
| | | CTCCCCACCCCCAATTTTGTATTTATTTATTTTTT | |
| | | AATTATTTTGTGCAGCGATGGGGGCGGGGGGG | |
| | | GGGGGGGGGGGGGCGCGCGCCAGGCGGGGC | |
| | | GGGGCGGGGCGAGGGGCGGGGCGGGGCGAG | |
| | | GCGGAGAGGTGCGGCGGCAGCCAATCAGAGCG | |
| | | GCGCGCTCCGAAAGTTTCCTTTTATGGCGAGGC | |
| | | GGCGGCGGCGGCGGCCCTATAAAAAGCGAAGC | |
| | | GCGCGGCGGGCGGGAGCGGGATCAGCCACCG | |
| | | CGG | |
| CK8 | 739 | CCACTACGGGTTTAGGCTGCCCATGTAAGGAGG | |
| | | CAAGGCCTGGGGACACCCGAGATGCCTGGTTAT | |
| | | AATTAACCCAGACATGTGGCTGCCCCCCCCCCC | |
| | | CCCAACACCTGCTGCCTCTAAAAATAACCCTGTC | |
| | | CCTGGTGGATCCCACTACGGGTTTAGGCTGCCC | |
| | | ATGTAAGGAGGCAAGGCCTGGGGACACCCGAG | |
| | | ATGCCTGGTTATAATTAACCCAGACATGTGGCTG | |
| | | CCCCCCCCCCCCCCAACACCTGCTGCCTCTAAA | |
| | | AATAACCCTGTCCCTGGTGGATCCCACTACGGG | |
| | | TTTAGGCTGCCCATGTAAGGAGGCAAGGCCTGG | |
| | | GGACACCCGAGATGCCTGGTTATAATTAACCCA | |
| | | GACATGTGGCTGCCCCCCCCCCCCCCAACACCT | |
| | | GCTGCCTCTAAAAATAACCCTGTCCCTGGTGGAT | |
| | | CCCCTGCATGCGAAGATCTTCGAACAAGGCTGT | |
| | | GGGGGACTGAGGGCAGGCTGTAACAGGCTTGG | |
| | | GGGCCAGGGCTTATACGTGCCTGGGACTCCCAA | |
| | | AGTATTACTGTTCCATGTTCCCGGCGAAGGGCC | |
| | | AGCTGTCCCCCGCCAGCTAGACTCAGCACTTAG | |
| | | TTTAGGAACCAGTGAGCAAGTCAGCCCTTGGGG | |
| | | CAGCCCATACAAGGCCATGGGGCTGGGCAAGCT | |
| | | GCACGCCTGGGTCCGGGGTGGGCACGGTGCCC | |
| | | GGGCAACGAGCTGAAAGCTCATCTGCTCTCAGG | |
| | | GGCCCCTCCCTGGGGACAGCCCCTCCTGGCTA | |
| | | GTCACACCCTGTAGGCTCCTCTATATAACCCAGG | |
| | | GGCACAGGGGCTGCCCTCATTCTACCACCACCT | |
| | | CCACAGCACAGACAGACACTCAGGAGCCAGCCA | |
| | | GCGTCGA | |
| mU1a | 740 | ATGGAGGCGGTACTATGTAGATGAGAATTCAGG | |
| | | AGCAAACTGGGAAAAGCAACTGCTTCCAAATATT | |
| | | TGTGATTTTTACAGTGTAGTTTTGGAAAAACTCTT | |
| | | AGCCTACCAATTCTTCTAAGTGTTTTAAAATGTG | |
| | | GGAGCCAGTACACATGAAGTTATAGAGTGTTTTA | |
| | | ATGAGGCTTAAATATTTACCGTAACTATGAAATG | |
| | | CTACGCATATCATGCTGTTCAGGCTCCGTGGCC | |
| | | ACGCAACTCATACT | |
| EF-1α | 741 | GGGCAGAGCGCACATCGCCCACAGTCCCCGAG | |
| | | AAGTTGGGGGGAGGGGTCGGCAATTGAACGGG | |
| | | TGCCTAGAGAAGGTGGCGCGGGGTAAACTGGG | |
| | | AAAGTGATGTCGTGTACTGGCTCCGCCTTTTTCC | |
| | | CGAGGGTGGGGGAGAACCGTATATAAGTGCAGT | |
| | | AGTCGCCGTGAACGTTCTTTTTCGCAACGGGTTT | |
| | | GCCGCCAGAACACAG | |
| TBG | 742 | GGGCTGGAAGCTACCTTTGACATCATTTCCTCTG | |
| | | CGAATGCATGTATAATTTCTACAGAACCTATTAG | |
| | | AAAGGATCACCCAGCCTCTGCTTTTGTACAACTT | |
| | | TCCCTTAAAAAACTGCCAATTCCACTGCTGTTTG | |
| | | GCCCAATAGTGAGAACTTTTTCCTGCTGCCTCTT | |
| | | GGTGCTTTTGCCTATGGCCCCTATTCTGCCTGCT | |
| | | GAAGACACTCTTGCCAGCATGGACTTAAACCCCT | |
| | | CCAGCTCTGACAATCCTCTTTCTCTTTTGTTTTAC | |
| | | ATGAAGGGTCTGGCAGCCAAAGCAATCACTCAA | |
| | | AGTTCAAACCTTATCATTTTTTGCTTTGTTCCTCT | |
| | | TGGCCTTGGTTTTGTACATCAGCTTTGAAAATAC | |
| | | CATCCCAGGGTTAATGCTGGGGTTAATTTATAAC | |
| | | TAAGAGTGCTCTAGTTTTGCAATACAGGACATGC | |
| | | TATAAAAATGGAAAGAT | |

In another example, a viral vector of the disclosure (e.g., an AAV vector) incorporates a ubiquitous promoter sequence capable of expressing an antisense construct of the disclosure. In one example, the ubiquitous promoter is an RNA Pol II or an RNA Pol III promoter. Exemplary Pol II and Pol III promoters are described in Preece et al. Gene Ther. 27:451-8(2020) and Jawdekar et al. Biochim. Biophys. Acta 1779(5):295-305 (2008), the disclosures of which are hereby incorporated by reference as they relate to RNA Pol II and RNA Pol III promoters. For example, the RNA Pol III promoter suitable for inclusion into the vector of the disclosure may be a U6 small nuclear 1 promoter, such as, a U6 small nuclear 1 promoter having a nucleic acid sequence of any one of SEQ ID NOs: 728-733 or 772 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of any one of SEQ ID NOs: 728-733 or 772.

In another example, the RNA Pol III promoter is an H1 promoter, such as an H1 promoter having a nucleic acid sequence of SEQ ID NO: 734 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 734.

In another example, the RNA Pol III promoter is a 7SK promoter, such as a 7SK promoter having a nucleic acid sequence of SEQ ID NO: 735 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 735.

In another example, the ubiquitous promoter is an apolipoprotein E (ApoE)-human alpha 1-antitrypsin (hAAT; ApoE-hAAT) promotoer, such as an ApoE-hAAT promoter having a nucleic acid sequence of SEQ ID NO: 736 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 736.

In another example, the ubiquitous promoter is a CAG promoter including a cytomegalovirus (CMV) early enhancer element, the promoter, first exon, and first intron of the chicken beta-actin gene, and the splice acceptor of the rabbit beta-globin gene, such as a CAG promoter having a nucleic acid sequence of SEQ ID NO: 737 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 737.

In another example, the ubiquitous promoter is a chicken beta actin (CBA) promoter, such as a CBA promoter having a nucleic acid sequence of SEQ ID NO: 738 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 738.

In another example, the ubiquitous promoter is a variant of a muscle creatine kinase promoter, the CK8 promoter, such as a CK8 promoter having a nucleic acid sequence of SEQ ID NO: 739 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 739.

In another example, the ubiquitous promoter is a mouse U1 small nuclear RNA (mU1a) promoter, such as a mU1a promoter having a nucleic acid sequence of SEQ ID NO: 740 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 740.

In another example, the ubiquitous promoter is an elongation factor 1 alpha (EF-1α) promoter, such as an EF-1α promoter having a nucleic acid sequence of SEQ ID NO: 741 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 741.

In another example, the ubiquitous promoter is a thyroxine binding globulin (TBG) promoter, such as a TBG promoter having a nucleic acid sequence of SEQ ID NO: 742 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO. 742.

Once a polynucleotide encoding the disclosed ASO agent has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, Calif.) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode Grik2-targeting ASO agents and additionally include a mammalian enhancer sequence. Many enhancer sequences are now known from mammalian genes, and examples are enhancers from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples are the SVenhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). An enhancer may be spliced into a vector containing a polynucleotide encoding an antisense construct of the disclosure, for example, at a position 5′ or 3′ to this gene. In a particular orientation, the enhancer is positioned at the 5′ side of the promoter, which in turn is located 5′ relative to the polynucleotide encoding an ASO agent of the disclosure. Non-limiting examples of enhancer sequences are provided in Table 7 below.

Additional regulatory elements that may be included in polynucleotides for use in the compositions and methods described herein are intron sequences. Intron sequences are non-protein-coding RNA sequences found in pre-mRNA which are removed during RNA splicing to produce the mature mRNA product. Intronic sequences are important for the regulation of gene expression in that they may be further processed to produce other non-coding RNA molecules. Alternative splicing, nonsense-mediated decay, and mRNA export are biological processes that have been shown to be regulated by intronic sequences. Intronic sequences may also facilitate the expression of a transgene through intron-mediated enhancement. Non-limiting examples of intron sequences are provided in Table 7 below.

Further regulatory elements that may be used in conjunction with the vectors of the disclosure include inverted terminal repeat (ITR) sequences. ITR sequences are found, e.g., in AAV genomes at the 5′ and 3′ ends, each typically containing about 145 base pairs. AAV ITR sequences are particularly important for AAV genome multiplication by facilitating complementary strand synthesis once an AAV vector is incorporated into a cell. Moreover, ITRs have been shown to be critical for integration of the AAV genome into the genome of the host cell and encapsidation of the AAV genome. Non-limiting examples of ITR sequences are provided in Table 7 below.

Additional regulatory elements suitable for incorporation into the vectors of the disclosure include polyadenylation sequences (i.e., polyA sequences). PolyA sequences are RNA tails containing a stretch of adenine bases. These sequences are appended to the 3′ end of an RNA molecule to produce a mature mRNA transcript. Several biological processes related to mRNA processing and transport are modulated by polyA sequences, including nuclear export, translation, and stability. In mammalian cells, shortening of the polyA tails results in increased likelihood of mRNA degradation. Non-limiting examples of a polyA sequence are provided in Table 7, below.

TABLE 7
Exemplary regulatory sequences

| | SEQ | |
|---|---|---|
| Regulatory element | ID NO | Nucleotide sequence |
| Chimeric Intron | 743 | GTAAGTATCAAGGTTACAAGACAGGTTTAAGGAGACCAATAG |
| | | AAACTGGGCTTGTCGAGACAGAGAAGACTCTTGCGTTTCTG |
| | | ATAGGCACCTATTGGTCTTACTGACATCCACTTTGCCTTTCT |
| | | CTCCACAG |
| VH4 Intron | 744 | GTGAGTATCTCAGGGATCCAGACATGGGGATATGGGAGGTG |
| | | CCTCTGATCCCAGGGCTCACTGTGGGTCTCTCTGTTCACAG |
| CMV Enhancer | 745 | CGTTACATAACTTACGGTAAATGGCCCGCCTGGCTGACCGC |
| | | CCAACGACCCCCGCCCATTGACGTCAATAATGACGTATGTT |
| | | CCCATAGTAACGCCAATAGGGACTTTCCATTGACGTCAATGG |
| | | GTGGAGTATTTACGGTAAACTGCCCACTTGGCAGTACATCAA |
| | | GTGTATCATATGCCAAGTACGCCCCCTATTGACGTCAATGAC |
| | | GGTAAATGGCCCGCCTGGCATTATGCCCAGTACATGACCTT |
| | | ATGGGACTTTCCTACTTGGCAGTACATCTACGTATTAGTCAT |
| | | CGCTATTACCATG |
| AAV 5′-ITR | 746 | CTGCGCGCTCGCTCGCTCACTGAGGCCGCCCGGGCAAAGC |
| | | CCGGGCGTCGGGCGACCTTTGGTCGCCCGGCCTCAGTGAG |
| | | CGAGCGAGCGCGCAGAGAGGGAGTGGCCAACTCCATCACT |
| | | AGGGGTTCCT |
| Modified AAV 5′-ITR | 747 | CTGCGCGCTCGCTCGCTCACTGAGGCCGCCCGGGCAAAGC |
| (Deleted D-sequence for | | CCGGGCGTCGGGCGACCTTTGGTCGCCCGGCCTCAGTGAG |
| self-complimentary | | CGAGCGAGCGCGCAGAGAGGGAGTGG |
| AAV) | | |
| AAV 3′-ITR 1 | 748 | GAACCCCTAGTGATGGAGTTGGCCACTCCCTCTCTGCGCGC |
| | | TCGCTCGCTCACTGAGGCCGCCCGGGCAAAGCCCGGGCGT |
| | | CGGGCGACCTTTGGTCGCCCGGCCTCAGTGAGCGAGCGAG |
| | | CGCGCAGAGAGGGAGTGGCCAA |
| AAV 3′-ITR 2 | 789 | AGGAACCCCTAGTGATGGAGTTGGCCACTCCCTCTCTGCGC |
| | | GCTCGCTCGCTCACTGAGGCCGGGCGACCAAAGGTCGCCC |
| | | GACGCCCGGGCTTTGCCCGGGCGGCCTCAGTGAGCGAGCG |
| | | AGCGCGCAG |
| Modified AAV 3′-ITR | 749 | TTGGCCACTCCCTCTCTGCGCGCTCGCTCGCTCACTGAGGC |
| (Deleted D-sequence for | | CGGGCGACCAAAGGTCGCCCGACGCCCGGGCTTTGCCCGG |
| self-complimentary | | GCGGCCTCAGTGAGCGAGCGAGCGCGCAG |
| AAV) | | |
| Rabbit β-globin (RBG) | 750 | ATAAAGGAAATTTATTTTCATTGCAATAGTGTGTTGGAATTTT |
| PolyA signal #1 | | TTGTGTCTCTCA |
| RBG PolyA #2 | 751 | GATCTTTTTCCCTCTGCCAAAAATTATGGGGACATCATGAAG |
| | | CCCCTTGAGCATCTGACTTCTGGCTAATAAAGGAAATTTATT |
| | | TTCATTGCAATAGTGTGTTGGAATTTTTTGTGTCTCTCACTCG |
| RBG PolyA #3 | 792 | GATCCGATCTTTTTCCCTCTGCCAAAAATTATGGGGACATCA |
| | | TGAAGCCCCTTGAGCATCTGACTTCTGGCTAATAAAGGAAAT |
| | | TTATTTTCATTGCAATAGTGTGTTGGAATTTTTTGTGTCTCTC |
| | | ACTCG |
| Bovine growth hormone | 793 | TAGCAGGCATGCTGGGGAG |
| (BGH) PolyA | | |

In other examples, a viral vector of the disclosure (e.g., an AAV vector) incorporates one or more regulatory sequence elements capable of facilitating the expression an antisense construct of the disclosure. In one example, the regulatory sequence element is an intron sequence. For example, an intron sequence suitable for inclusion into the vector of the disclosure may be a chimeric intron such as a chimeric intron having a nucleic acid sequence of SEQ ID NO: 743 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 743.

In another example, the intron sequence is an immunoglobulin heavy-chain-variable 4 (VH4) intron, such as a VH4 sequence having a nucleic acid sequence of SEQ ID NO: 744 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 744.

In another example, the regulatory sequence element is an enhancer sequence. For example, the enhancer sequence may be a CMV enhancer, such as a CMV enhancer having a nucleic acid sequence of SEQ ID NO: 745 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 745.

In another example, the regulatory sequence element is an ITR sequence, such as, e.g., an AAV ITR sequence. For example, the ITR sequence may be an AAV 5′ ITR sequence, such as an AAV 5′ ITR sequence having a nucleic acid sequence of SEQ ID NO: 746 or SEQ ID NO: 747 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 746 or SEQ ID NO: 747.

In another example, the ITR sequence is an AAV 3′ ITR sequence, such as an AAV 3′ ITR sequence having a nucleic acid sequence of SEQ ID NO: 748 or SEQ ID NO: 749 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NO: 748 or SEQ ID NO: 749.

In another example, the regulatory sequence element is a polyadenylation signal (i.e., a polyA tail). For example, the polyadenylation signal suitable for use with the vectors disclosed herein include a rabbit β-globin (RBG) polyadenylation signal, such as a RBG polyadenylation signal having a nucleic acid sequence of SEQ ID NO: 750, SEQ ID NO: 751, or SEQ ID NO: 792 or a variant thereof having at least 70% (e.g., at least 71%, 75%, 80%, 85%, 90%, 91%, 92%, 93%, 94%, 95%, 96%, 97%, 98%, 99% or more) sequence identity to the nucleic acid sequence of SEQ ID NO: 750, SEQ ID NO: 751, or SEQ ID NO: 792. Another polyadenylation signal that can be used in conjunction with the disclosed compositions and methods is a bovine growth hormone (BGH) polyadenylation signal, such as a BGH polyadenylation signal of SEQ ID NO: 793 or a variant thereof having at least 70% (e.g., at least 71%, 75%, 80%, 85%, 90%, 91%, 92%, 93%, 94%, 95%, 96%, 97%, 98%, 99% or more) sequence identity to the nucleic acid sequence of SEQ ID NO: 793.

### Viral Vectors

Viral genomes provide a rich source of vectors that can be used for the efficient delivery of exogenous polynucleotides into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a parvovirus (e.g., adeno-associated viruses (AAV)), retrovirus (e.g., Retroviridae family viral vector), adenovirus (e.g., Ad5, Ad26, Ad34, Ad35, and Ad48), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, murine mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### AAV Vectors

Nucleic acids of the compositions described herein may be incorporated into an AAV vector and/or an AAV virion in order to facilitate their introduction into a cell, e.g., in connection with the methods disclosed herein. AAV vectors can be used in the central nervous system, and appropriate promoters and serotypes are discussed in, e.g., Pignataro et al., J Neural Transm 125(3):575-89 (2017), the disclosure of which is incorporated herein by reference as it pertains to promoters and AAV serotypes useful in CNS gene therapy. rAAV vectors useful in the compositions and methods described herein are recombinant nucleic acid constructs that include (1) a heterologous sequence to be expressed (e.g., a polynucleotide encoding a Grik2 mRNA-targeting ASO agent) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

Examples of AAVs that can be used as a vector for incorporating an ASO agent of the disclosure (e.g., siRNA, shRNA, miRNA, or shmiRNA, or shmiRNA described herein) include, e.g., AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAVIK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10 , AAV.HSC11, AAV.HSC12, AAV.HSC13, AAV.HSC14, AAV.HSC15, AAV-TT, AAV-DJ8, and AAV.HSC16.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and U.S. Pat. No.6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAVmay be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype pseudotyped with a capsid gene derived from a serotype other than the given serotype (AAV1, AAV2, AAV3, AAV4, AAVS, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAV.LK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10 , AAV.HSC11, AAV.HSC12, AAV.HSC13, AAV.HSC14, AAV.HSC15, AAV-TT, AAV-DJ8, or AAV.HSC16). For example, the AAV may include a pseudotyped recombinant AAV (rAAV) vector, such as, e.g., an rAAV2/8 or rAAV2/9 vector. Methods for producing and using pseudotyped rAAV are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

The rAAV used in the compositions and methods of the disclosure may include a capsid protein from an AAV capsid serotype selected from AAV1, AAV2, AAV3, AAV4, AAVS, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAVIK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10 , AAV.HSC11, AAV.HSC12, AAV.HSC13, AAV.HSC14, AAV.HSC15, AAV.HSC16, AAV-TT, AAVDJ8, or a derivative, modification, or pseudotype thereof, such as, e.g., a capsid protein that is at least 80% or more identical, e.g., 85%, 85%, 87%, 88%, 89%, 90%, 91%, 92%, 93%, 94%, 95%, 96%, 97%, 98%, 99%, 99.5%, or more, i.e., up to 100% identical, to e.g., vp1, vp2 and/or vp3 sequence of an AAV capsid serotype selected from AAV1, AAV2, AAV3, AAV4, AAVS, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAVIK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10 , AAV.HSC11, AAV.HSC12, AAV.HSC13, AAV.HSC14, AAV.HSC15, AAV-TT, AAV-DJ8 or AAV.HSC16.

The AAV vector, which can be used in the methods described herein, may be an Anc80 or Anc80L65 vector, as described in Zinn et al., 2015: 1056-1068, which is incorporated by reference in its entirety. The AAV vector may include one of the following amino acid insertions: LGETTRP (SEQ ID NO: 14 of '956, '517, '282, or '323) or LALGETTRP (SEQ ID NO: of '956, '517, '282, or '323), as described in U.S. Pat. Nos. 9,193,956; 9458517; and 9,587,282 and US patent application publication no. 2016/0376323, each of which is incorporated herein by reference in its entirety. Alternatively, AAV vector used in the methods described herein may be an AAV.7m8, as described in U.S. Pat. Nos. 9,193,956; 9,458,517; and 9,587,282 and US patent application publication no. 2016/0376323, each of which is incorporated herein by reference in its entirety. Further still, the AAV vector used in the methods described herein may be any AAV disclosed in U.S. Pat. No. 9,585,971, such as an AAV-PHP.B vector. Another AAV vector used in methods described herein may be any vector disclosed in Chan et al. (Nat Neurosci. 20(8):1172-1179, 2017), such as an AAV.PHP.eB, which comprises an AAV9 capsid protein having a peptide inserted between amino acid positions 588 and 589 and modifications A587D/588G. Furthermore, the AAV vector used in the methods described herein may be any AAV disclosed in U.S. Pat. No. 9,840,719 and WO 2015/013313, such as an AAV.Rh74 or RHM4-1 vector, each of which is incorporated herein by reference in its entirety. Additionally, the AAV vector used in the methods described herein may be any AAV disclosed in WO 2014/172669, such as AAV rh.74, which is incorporated herein by reference in its entirety. The AAV vector used in the methods described herein may also be an AAV2/5 vector, as described in Georgiadis et al., 2016, Gene Therapy 23: 857-862 and Georgiadis et al., 2018, Gene Therapy 25: 450, each of which is incorporated by reference in its entirety. In further examples, the AAV vector used in the methods described herein may be any AAV disclosed in WO 2017/070491, such as an AAV2tYF vector, which is incorporated herein by reference in its entirety. Additionally, AAV vector used in the methods described herein may be an AAVLKO3 or AAV3B vector, as described in Puzzo et al., 2017, Sci. Transl. Med. 29(9): 418, which is incorporated by reference in its entirety. In additional examples, the AAV vector used in the methods described herein may be any AAV disclosed in U.S. Pat. Nos. 8,628,966; 8,927,514; 9,923,120 and WO 2016/049230, such as HSC1, HSC2, HSC3, HSC4, HSCS, HSC6, HSC7, HSC8, HSC9, HSC10, HSC11, HSC12, HSC13, HSC14, HSC15, or HSC16, each of which is incorporated by reference in its entirety.

Furthermore, the AAV vector used in the methods described herein may be an AAV vector disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9458517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. The rAAV vector may have a capsid protein at least 80% or more identical, e.g., 85%, 85%, 87%, 88%, 89%, 90%, 91%, 92%, 93%, 94%, 95%, 96%, 97%, 98%, 99%, 99.5%, or more to the vp1, vp2 and/or vp3 amino acid sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9458517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

Additionally, the rAAV vector may have a capsid protein disclosed in Intl. Appl. Publ. No. WO 2003/052051 (see, e.g., SEQ ID NO: 2 of '051), WO 2005/033321 (see, e.g., SEQ ID NOs: 123 and 88 of '321), WO 03/042397 (see, e.g., SEQ ID NOs: 2, 81, 85, and 97 of '397), WO 2006/068888 (see, e.g., SEQ ID NOs: 1 and 3-6 of '888), WO 2006/110689, (see, e.g., SEQ ID NOs: 5-38 of '689) WO2009/104964 (see, e.g., SEQ ID NOs: 1-5, 7, 9, 20, 22, 24 and 31 of '964), WO 2010/127097 (see, e.g., SEQ ID NOs: 5-38 of '097), and WO 2015/191508 (see, e.g., SEQ ID NOs: 80-294 of '058), and U.S. Appl. Publ. No. 20150023924 (see, e.g., SEQ ID NOs: 1, 5-of '924), the contents of each of which is herein incorporated by reference in its entirety, such as, e.g., an rAAV vector having a capsid protein that is at least 80% or more identical, e.g., 85%, 85%, 87%, 88%, 89%, 90%, 91%, 92%, 93%, 94%, 95%, 96%, 97%, 98%, 99%, 99.5%, or more to the vp1, vp2 and/or vp3 amino acid sequence of an AAV capsid disclosed in Intl. Appl. Publ. No. WO 2003/052051 (see, e.g., SEQ ID NO: 2 of '051), WO 2005/033321 (see, e.g., SEQ ID NOs: 123 and 88 of '321), WO 03/042397 (see, e.g., SEQ ID NOs: 2, 81, 85, and 97 of '397), WO 2006/068888 (see, e.g., SEQ ID NOs: 1 and 3-6 of '888), WO 2006/110689 (see, e.g., SEQ ID NOs: 5-38 of 689) WO 2009/104964 (see, e.g., SEQ ID NOs: 1-5, 7, 9, 20, 22, 24 and 31 of '964), WO 2010/127097 (see, e.g., SEQ ID NOs: 5-38 of '097), and WO 201 5/1 91 508 (see, e.g., SEQ ID NOs: 80-294 of '508), and U.S. Appl. Publ. No. 20150023924 (see, e.g., SEQ ID NOs: 1, 5-of '924).

Nucleic acid sequences of AAV-based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9458517; 15 and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO 2009/104964, WO 2010/127097, and WO 2015/191508, and U.S. Appl. Publ. No. 20150023924.

Accordingly, the rAAV vector may include a capsid containing a capsid protein from two or more AAV capsid serotypes, such as, e.g., AAV serotypes selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAVIK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16.

A single-stranded AAV (ssAAV) vector can be used in conjunction with the disclosed methods and compositions. Alternatively, a self-complementary AAV vector (scAAV) can be used (see, e.g., Wu, 2007, Human Gene Therapy, 18(2):171-82, McCarty et al, 2001, Gene Therapy, Vol. 8, Number 16, Pages 1248-1254; and U.S. Patent Nos. 6,596,535; 7,125,717; and 7,456,683, each of which is incorporated herein by reference in its entirety).

A recombinant AAV vector with a tropism for cells in the central nervous system, including but not limited to neurons and/or glial cells, can be used for delivering a polynucleotide agent (e.g., an ASO agent) of the disclosure. Such vectors can include non-replicating “rAAV” vectors, particularly those bearing an AAV9 or AAVrhcapsid. AAV variant capsids can be used, including but not limited to those described by Wilson in US Patent No. 7,906,111, which is incorporated by reference herein in its entirety, with AAV/hu.31 and AAV/hu.32 being particularly preferred, as well as AAV variant capsids described by Chatterjee in U.S. Pat. Nos. 8,628,966, 8,927,514 and Smith et al., 2014, Mol Ther 22: 1625-1634, each of which is incorporated by reference herein in its entirety. Furthermore, the AAV-TT vector disclosed by Tordo et al. (Brain 141:2014-31, 2018; incorporated herein by reference in its entirety), which incorporates amino acid sequences that are conserved among natural AAV2 isolates, may also be used in conjunction with the compositions and methods of the disclosure. AAV-TT variant capsids exhibit enhanced neurotropism and robust distribution throughout the CNS compared to AAV2, AAV9, and AAVrh10. Similarly, the AAV-DJ8 vector disclosed in Hammond et al. (PLoS ONE 12(2):e0188830, 2017; incorporated by reference herein in its entirety) exhibits superior neurotropism and may be suitable for use with the compositions and methods of the disclosure.

In a particular example, the disclosure features AAV9 vectors, including an artificial genome including (i) an expression cassette containing the polynucleotide encoding an ASO sequence (e.g., any one of SEQ ID NOs: 1-100) under the control of regulatory elements and flanked by ITRs; and (ii) a viral capsid that has the amino acid sequence of the AAV9 capsid protein or is at least 95%, 96%, 97%, 98%, 99% or 99.9% identical to the amino acid sequence of the AAV9 capsid protein while retaining the biological function of the AAV9 capsid. The encoded AAV9 capsid may have the sequence of SEQ ID NO: 116 set forth in U.S. Pat. No. 7,906,111 which is incorporated by reference herein in its entirety, with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV9 capsid.

Also provided herein are AAVrhvectors including an artificial genome including (i) an expression cassette containing the polynucleotide under the control of regulatory elements and flanked by ITRs; and (ii) a viral capsid that has the amino acid sequence of the AAVrhcapsid protein or is at least 95%, 96%, 97%, 98%, 99% or 99.9% identical to the amino acid sequence of the AAVrhcapsid protein while retaining the biological function of the AAVrh1Ocapsid. The encoded AAVrhcapsid may have the sequence of SEQ ID NO: 81 set forth in U.S. Pat. No. 9,790,427 which is incorporated by reference herein in its entirety, with 1, 2, 3, 4, 5, 6, 7, 8, 9, 10, 11, 12, 13, 14, 15, 16, 17, 18, 19, 20, 21, 22, 23, 24, 25, 26, 27, 28, 29 or amino acid substitutions and retaining the biological function of the AAVrh capsid.

Gene regulatory elements may be selected to be functional in a mammalian cell (e.g., a neuron). The resulting construct which contains the operatively linked components is flanked by (5′ and 3′) functional AAV ITR sequences. Particular examples include vectors derived from AAV serotypes having tropism for and high transduction efficiencies in cells of the mammalian CNS, particularly neurons. A review and comparison of transduction efficiencies of different serotypes is provided in this patent application. In certain examples, AAV2, AAVS, AAV9 and AAVrhbased vectors direct long-term expression of polynucleotides in CNS, for example, by transducing neurons and/or glial cells.

The AAV expression vector which harbors the polynucleotide of interest (e.g., a polynucleotide encoding an ASO agent described herein) flanked by AAV ITRs can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173, 414 and 5,139,941; International Publications Nos. WO 92/01070 (published 23 Jan. 1992) and WO 93/03769 (published 4 Mar. 1993). Alternatively, AAV ITRs can be excised from the viral genome or from an AAV vector containing the same and fused to the 5′ and 3′ ends of a selected nucleic acid construct that is present in another vector using standard nucleic acid ligation techniques. AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ relative to one or more selected nucleic acid sequences. Preferred codons for expression of the chimeric gene sequence in mammalian CNS cells can be used, and in certain cases, codon optimization of the polynucleotide can be performed by well-known methods. The complete chimeric sequence is assembled from overlapping polynucleotides prepared by standard methods. In order to produce AAV virions, an AAV expression vector is introduced into a suitable host cell using known techniques, such as by transfection. A number of transfection techniques are generally known in the art. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

For instance, a particular viral vector of the disclosure may include, in addition to a nucleic acid sequence of the disclosure (e.g., any one of SEQ ID NOs: 1-100), the backbone of AAV vector plasmid with ITR derived from an AAV2 virus, a promoter such as, e.g., a U6 small nuclear 1 promoter or variants thereof, H1 promoter, 7SK promoter, ApoE-hAAT promoter, CBA promoter, CK8 promoter, mU1a promoter, EF-1α promoter, TBG promoter, murine PGK promoter or the CAG promoter, or any neuronal promoter such as the hSyn promoter, NeuN promoter, CaMKII promoter, Ta-1 promoter, NSE promoter, PDGFβ promoter, VGLUT promoter, SST promoter, NPY promoter, VIP promoter, PV promoter, GAD65 or GAD67 promoter, DRD1 promoter, DRD2 promoter, MAP1 B promoter, C1ql2 promoter, POMC promoter, or Prox1 promoter, with or without the wild-type or mutant form of the WPRE, and a rabbit beta-globin polyA sequence (see Table and Table 6).

The present disclosure further relates to an rAAV including (i) an expression cassette containing a polynucleotide under the control of regulatory elements and flanked by ITRs, and (ii) an AAV capsid, wherein the polynucleotide encodes an inhibitory RNA (e.g., an ASO, such as, e.g., siRNA, shRNA, miRNA, or shmiRNA, or shmiRNA, and, in particular, an ASO having a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100) that specifically binds to at least a portion or region of a Grik2 mRNA (e.g., any one of the portions or regions of a Grik2 mRNA described in SEQ ID NOs: 115-681) and that inhibits (e.g., knocks down) expression of GluK2 protein in a cell (e.g., a neuron).

The AAV vector may include, e.g., an ASO (e.g., siRNA, shRNA, miRNA, or shmiRNA, or shmiRNA) sequence that that binds to the Grik2 mRNA, and a hSyn promoter. For example, the AAV vector may contain nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an hSyn promoter (e.g., hSyn promoter having a nucleic acid sequence of any one of SEQ ID NO: 682-685 and SEQ ID NO: 790 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 682-685 or SEQ ID NO: 790).

Alternatively, the AAV vector may include an ASO (e.g., siRNA, shRNA, miRNA, or shmiRNA) sequence that binds to and inhibits the expression of the Grik2 mRNA, and a NeuN promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an NeuN promoter (e.g., NeuN promoter having a nucleic acid sequence of SEQ ID NO: 686 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 686).

Alternatively, the AAV vector may include an ASO (e.g., siRNA, shRNA, miRNA, or shmiRNA) sequence that binds to and inhibits the expression of the Grik2 mRNA, and a CaMKII promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 10 94%, 95%, 96%, 97%, 98%, 99%, or more) sequence identity to the nucleic acid sequence of any one of SEQ ID NOs: 1-100 and an CaMKII promoter (e.g., CaMKII promoter having a nucleic acid sequence of any one of any one of SEQ ID NOs: 687-691 and SEQ ID NO: 802 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 687-691 and SEQ ID NO: 802).

Alternatively, the AAV vector may include an ASO (e.g., siRNA, shRNA, miRNA, or shmiRNA) sequence that binds to and inhibits the expression of the Grik2 mRNA, and a NSE promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 20 94%, 95%, 96%, 97%, 98%, 99%, or more) sequence identity to the nucleic acid sequence of any one of

SEQ ID NOs: 1-100 and an NSE promoter (e.g., NSE promoter having a nucleic acid sequence of SEQ ID NOs: 692 or 693 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 692 or 693).

Alternatively, the AAV vector may include an ASO (e.g., siRNA, shRNA, miRNA, or shmiRNA) sequence that binds to and inhibits the expression of the Grik2 mRNA, and a PDGF 3 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an PDGF 3 promoter (e.g., PDGF(3 promoter having a nucleic acid sequence of any one of SEQ ID NOs: 694-696 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 694-696).

Alternatively, the AAV vector may include an ASO (e.g., siRNA, shRNA, miRNA, or shmiRNA) sequence that binds to and inhibits the expression of the Grik2 mRNA, and a VGIuT promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an VGIuT promoter (e.g., VGIuT promoter having a nucleic acid sequence of any one of SEQ ID NOs: 697-701 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 708-712).

Alternatively, the AAV vector may include an ASO (e.g., siRNA, shRNA, miRNA, or shmiRNA) sequence that binds to and inhibits the expression of the Grik2 mRNA, and a SST promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 5 94%, 95%, 96%, 97%, 98%, 99%, or more) sequence identity to the nucleic acid sequence of any one of

SEQ ID NOs: 1-100 and an SST promoter (e.g., SST promoter having a nucleic acid sequence of SEQ ID NO: 702 or SEQ ID NO: 703 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 702 or SEQ ID NO: 703).

Alternatively, the AAV vector may include an ASO (e.g., siRNA, shRNA, miRNA, or shmiRNA) sequence that binds to and inhibits the expression of the Grik2 mRNA, and a NPY promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an NPY promoter (e.g., NPY promoter having a nucleic acid sequence of SEQ ID NO: 704 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 704).

Alternatively, the AAV vector may include an ASO (e.g., siRNA, shRNA, miRNA, or shmiRNA) sequence that binds to and inhibits the expression of the Grik2 mRNA, and a VIP promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an VIP promoter (e.g., VIP promoter having a nucleic acid sequence of SEQ ID NO: 25 705 or SEQ ID NO: 706 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 705 or SEQ ID NO: 706).

Alternatively, the AAV vector may include an ASO (e.g., siRNA, shRNA, miRNA, or shmiRNA) sequence that binds to and inhibits the expression of the Grik2 mRNA, and a PV promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an PV promoter (e.g., PV promoter having a nucleic acid sequence of any one of SEQ ID NOs: 707-709 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 707-709).

Alternatively, the AAV vector may include an ASO (e.g., siRNA, shRNA, miRNA, or shmiRNA) sequence that binds to and inhibits the expression of the Grik2 mRNA, and a GAD65 promoter. For example, the disclosed AAV vector may include a nucleic acid sequence of any one of SEQ ID NOs: 1-40 100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an GAD65 promoter (e.g., GAD65 promoter having a nucleic acid sequence of any one of SEQ ID NOs: 710-713 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 710-713).

Alternatively, the AAV vector may include an ASO (e.g., siRNA, shRNA, miRNA, or shmiRNA) sequence that binds to and inhibits the expression of the Grik2 mRNA, and a GAD67 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an GAD67 promoter (e.g., GAD67 promoter having a nucleic acid sequence of SEQ ID NO: 714 or SEQ ID NO: 715 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 714 or 715).

Alternatively, the AAV vector may include an ASO (e.g., siRNA, shRNA, miRNA, or shmiRNA) sequence that binds to and inhibits the expression of the Grik2 mRNA, and a DRD1 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an DRD1 promoter (e.g., DRD1 promoter having a nucleic acid sequence of SEQ ID NO: 716 or a variant thereof having at least 85% (at least 85%, 86%, 87%, 88%, 89%, 90%, 20 91%, 92%, 93%, 94%, 95%, 96%, 97%, 98%, 99%, or more) sequence identity to the nucleic acid sequence of SEQ ID NO: 716).

Alternatively, the AAV vector may include an ASO (e.g., siRNA, shRNA, miRNA, or shmiRNA) sequence that binds to and inhibits the expression of the Grik2 mRNA, and a DRD2 promoter. For example, the disclosed AAV vector may include a nucleic acid sequence of any one of SEQ ID NOs: 1-25 100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an DRD2 promoter (e.g., DRD2 promoter having a nucleic acid sequence of SEQ ID NO: 717 or SEQ ID NO: 718 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 717 or SEQ ID NO: 718).

Alternatively, the AAV vector may include an ASO (e.g., siRNA, shRNA, miRNA, or shmiRNA) sequence that binds to and inhibits the expression of the Grik2 mRNA, and a C1ql2 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of

SEQ ID NOs: 1-100 and an C1ql2 promoter (e.g., C1ql2 promoter having a nucleic acid sequence of SEQ ID NO: 719 or SEQ ID NO: 791 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 719 or SEQ ID NO: 791).

Alternatively, the AAV vector may include an ASO (e.g., siRNA, shRNA, miRNA, or shmiRNA) sequence that binds to and inhibits the expression of the Grik2 mRNA, and a POMC promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an POMC promoter (e.g., POMC promoter having a nucleic acid sequence of SEQ ID NO: 720 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 720).

Alternatively, the AAV vector may include an ASO (e.g., siRNA, shRNA, miRNA, or shmiRNA) sequence that binds to and inhibits the expression of the Grik2 mRNA, and a PROX1 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an PROX1 promoter (e.g., PROX1 promoter having a nucleic acid sequence of SEQ ID NO: 721 or SEQ ID NO: 722 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 721 or SEQ ID NO: 722).

Alternatively, the AAV vector may include an ASO (e.g., siRNA, shRNA, miRNA, or shmiRNA) sequence that binds to and inhibits the expression of the Grik2 mRNA, and a MAP1 B promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of

SEQ ID NOs: 1-100 and an MAP1B promoter (e.g., MAP1B promoter having a nucleic acid sequence of any one of SEQ ID NOs: 723-725 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 723-725). Alternatively, the AAV vector may include an ASO (e.g., siRNA, shRNA, miRNA, or shmiRNA) sequence that binds to and inhibits the expression of the Grik2 mRNA, and a Ta-1 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an Ta-1 promoter (e.g., Ta-1 promoter having a nucleic acid sequence of SEQ

ID NO: 726 or SEQ ID NO: 727 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 726 or SEQ ID NO: 727).

Alternatively, the AAV vector may include an ASO (e.g., siRNA, shRNA, miRNA, or shmiRNA) sequence that binds to and inhibits the expression of the Grik2 mRNA, and a U6 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an U6 promoter, such as a U6 promoter having a nucleic acid sequence of any one of SEQ ID NOs: 728-733 or 772 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 728-733, or 772.

Alternatively, the AAV vector may include an ASO (e.g., siRNA, shRNA, miRNA, or shmiRNA) sequence that binds to and inhibits the expression of the Grik2 mRNA, and an H1 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of

SEQ ID NOs: 1-100 and an H1 promoter, such as an H1 promoter having a nucleic acid sequence of SEQ ID NO: 734 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 734.

Alternatively, the AAV vector may include an ASO (e.g., siRNA, shRNA, miRNA, or shmiRNA)equence that binds to and inhibits the expression of the Grik2 mRNA, and an 7SK promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an 7SK promoter, such as an 7SK promoter having a nucleic acid sequence of

SEQ ID NO: 735 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 735.

Alternatively, the AAV vector may include an ASO (e.g., siRNA, shRNA, miRNA, or shmiRNA) sequence that binds to and inhibits the expression of the Grik2 mRNA, and an ApoE-hAAT promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an ApoE-hAAT promoter, such as an ApoE-hAAT promoter having a nucleic acid sequence of SEQ ID NO: 736 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 736.

Alternatively, the AAV vector may include an ASO (e.g., siRNA, shRNA, miRNA, or shmiRNA) sequence that binds to and inhibits the expression of the Grik2 mRNA, and a CAG promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an CAG promoter, such as a CAG promoter having a nucleic acid sequence of SEQ ID NO: 737 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 737.

Alternatively, the AAV vector may include an ASO (e.g., siRNA, shRNA, miRNA, or shmiRNA) sequence that binds to and inhibits the expression of the Grik2 mRNA, and a CBA promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and a CBA promoter, such as a CBA promoter having a nucleic acid sequence of SEQ ID NO: 738 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 738.

Alternatively, the AAV vector may include an ASO (e.g., siRNA, shRNA, miRNA, or shmiRNA) sequence that binds to and inhibits the expression of the Grik2 mRNA, and a CK8 promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and a CK8 promoter, such as CK8 promoter having a nucleic acid sequence of SEQ ID NO: 739 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 739.

Alternatively, the AAV vector may include an ASO (e.g., siRNA, shRNA, miRNA, or shmiRNA) sequence that binds to and inhibits the expression of the Grik2 mRNA, and an mU1a promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an mU1a promoter, such as an mU1a promoter having a nucleic acid sequence of SEQ ID NO: 740 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 740.

Alternatively, the AAV vector may include an ASO (e.g., siRNA, shRNA, miRNA, or shmiRNA) sequence that binds to and inhibits the expression of the Grik2 mRNA, and an EF-1a promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an EF-1a promoter, such as an EF-1a promoter having a nucleic acid sequence of SEQ ID NO: 741 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 741.

Alternatively, the AAV vector may include an ASO (e.g., siRNA, shRNA, miRNA, or shmiRNA) sequence that binds to and inhibits the expression of the Grik2 mRNA, and a TBG promoter. For example, the disclosed AAV vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of

SEQ ID NOs: 1-100 and a TBG promoter, such as TBG promoter having a nucleic acid sequence of SEQ ID NO: 742 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 742.

Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the polynucleotide. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the polynucleotide of interest is accommodated. In particular, the recombinant lentivirus are recovered through the in trans co-expression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR' backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5^{.}-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). Optionally, one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of polynucleotide expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polynucleotides are useful. The vector used in the methods and compositions described herein may include multiple promoters that permit expression more than one polynucleotide. Other elements that permit expression of multiple polynucleotides identified in the future are useful and may be utilized in the vectors suitable for use with the compositions and methods described herein. The vector used in the methods and compositions described herein may, be a clinical grade vector.

Accordingly, retroviral vectors may be employed in conjunction with the disclosed methods and compositions. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of specific viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (e.g., by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types.

Additionally, lentiviral vectors may be employed in combination with the methods and compositions disclosed herein. Accordingly, an object of the disclosure relates to a lentiviral vector including an ASO (e.g., siRNA, shRNA, miRNA, or shmiRNA, or shmiRNA) sequence (e.g., any one of the ASO sequences described in SEQ ID NOs: 1-100) that binds to and inhibits the expression of the Grik2 mRNA.

Accordingly, the lentiviral vector may include the nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100. The lentiviral vector may include an ASO sequence (e.g., siRNA, shRNA, miRNA, or shmiRNA, or shmiRNA) that binds to and inhibits the expression of the Grik2 mRNA, and a hsyn promoter.

The lentiviral vector may include, e.g., an ASO (e.g., siRNA, shRNA, miRNA, or shmiRNA, or shmiRNA) sequence that that binds to the Grik2 mRNA, and a hSyn promoter. For example, the lentiviral vector may contain nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an hSyn promoter (e.g., hSyn promoter having a nucleic acid sequence of any one of SEQ ID NOs: 682-685 and SEQ ID NO: 790 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 682-685 and SEQ ID NO: 790).

Alternatively, the lentiviral vector may include an ASO (e.g., siRNA, shRNA, miRNA, or shmiRNA) sequence that binds to and inhibits the expression of the Grik2 mRNA, and a NeuN promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 5 93%, 94%, 95%, 96%, 97%, 98%, 99%, or more) sequence identity to the nucleic acid sequence of any one of SEQ ID NOs: 1-100 and an NeuN promoter (e.g., NeuN promoter having a nucleic acid sequence of SEQ ID NO: 686 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 686).

Alternatively, the lentiviral vector may include an ASO (e.g., siRNA, shRNA, miRNA, or shmiRNA) sequence that binds to and inhibits the expression of the Grik2 mRNA, and a CaMKII promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an CaMKII promoter (e.g., CaMKII promoter having a nucleic acid sequence of any one of any one of SEQ ID NOs: 687-691 and SEQ ID NO: 802 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 687-691 and SEQ ID NO: 802).

Alternatively, the lentiviral vector may include an ASO (e.g., siRNA, shRNA, miRNA, or shmiRNA) sequence that binds to and inhibits the expression of the Grik2 mRNA, and a NSE promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an NSE promoter (e.g., NSE promoter having a nucleic acid sequence of SEQ ID NOs: 692 or SEQ ID NO: 693 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 692 or SEQ ID NO: 693).

Alternatively, the lentiviral vector may include an ASO (e.g., siRNA, shRNA, miRNA, or shmiRNA) sequence that binds to and inhibits the expression of the Grik2 mRNA, and a PDGF 3 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an PDGFP promoter (e.g., PDGF(3 promoter having a nucleic acid sequence of any one of SEQ ID NOs: 694-696 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 694-696).

Alternatively, the lentiviral vector may include an ASO (e.g., siRNA, shRNA, miRNA, or shmiRNA) sequence that binds to and inhibits the expression of the Grik2 mRNA, and a VGIuT promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an VGIuT promoter (e.g., VGIuT promoter having a nucleic acid sequence of any one of SEQ ID NOs: 697-701 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 697-701). Alternatively, the lentiviral vector may include an ASO (e.g., siRNA, shRNA, miRNA, or shmiRNA) sequence that binds to and inhibits the expression of the Grik2 mRNA, and a SST promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an SST promoter (e.g., SST promoter having a nucleic acid sequence of any one of SEQ ID NO: 702 or SEQ ID NO: 703 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NO: 702 or SEQ ID NO: 703).

Alternatively, the lentiviral vector may include an ASO (e.g., siRNA, shRNA, miRNA, or shmiRNA) sequence that binds to and inhibits the expression of the Grik2 mRNA, and a NPY promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an NPY promoter (e.g., NPY promoter having a nucleic acid sequence of SEQ ID NO: 704 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 704).

Alternatively, the lentiviral vector may include an ASO (e.g., siRNA, shRNA, miRNA, or shmiRNA) sequence that binds to and inhibits the expression of the Grik2 mRNA, and a VIP promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an VIP promoter (e.g., VIP promoter having a nucleic acid sequence of SEQ ID NO: 705 or SEQ ID NO: 706 or a variant thereof having at least 85% (at least 85%, 86%, 87%, 88%, 89%, 30 90%, 91%, 92%, 93%, 94%, 95%, 96%, 97%, 98%, 99%, or more) sequence identity to the nucleic acid sequence of any one of SEQ ID NO: 705 or SEQ ID NO: 706).

Alternatively, the lentiviral vector may include an ASO (e.g., siRNA, shRNA, miRNA, or shmiRNA) sequence that binds to and inhibits the expression of the Grik2 mRNA, and a PV promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an PV promoter (e.g., PV promoter having a nucleic acid sequence of any one of SEQ ID NOs: 707-709or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 707-709).

Alternatively, the lentiviral vector may include an ASO (e.g., siRNA, shRNA, miRNA, or shmiRNA) sequence that binds to and inhibits the expression of the Grik2 mRNA, and a GAD65 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an GAD65 promoter (e.g., GAD65 promoter having a nucleic acid sequence of any one of SEQ ID NOs: 710-713 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 710-713).

Alternatively, the lentiviral vector may include an ASO (e.g., siRNA, shRNA, miRNA, or shmiRNA) sequence that binds to and inhibits the expression of the Grik2 mRNA, and a GAD67 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an GAD67 promoter (e.g., GAD67 promoter having a nucleic acid sequence of SEQ ID NO: 714 or SEQ ID NO: 715 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 714 or SEQ ID NO: 715).

Alternatively, the lentiviral vector may include an ASO (e.g., siRNA, shRNA, miRNA, or shmiRNA) sequence that binds to and inhibits the expression of the Grik2 mRNA, and a DRD1 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an DRD1 promoter (e.g., DRD1 promoter having a nucleic acid sequence of SEQ ID NO: 716 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 716).

Alternatively, the lentiviral vector may include an ASO (e.g., siRNA, shRNA, miRNA, or shmiRNA) sequence that binds to and inhibits the expression of the Grik2 mRNA, and a DRD2 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an DRD2 promoter (e.g., DRD2 promoter having a nucleic acid sequence of SEQ ID NO: 717 or SEQ ID NO: 718 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 717 or SEQ ID NO: 718).

Alternatively, the lentiviral vector may include an ASO (e.g., siRNA, shRNA, miRNA, or shmiRNA) sequence that binds to and inhibits the expression of the Grik2 mRNA, and a C1ql2 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an C1ql2 promoter (e.g., C1ql2 promoter having a nucleic acid sequence of SEQ ID NO: 719 or SEQ ID NO: 791 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 719 or SEQ ID NO: 791).

Alternatively, the lentiviral vector may include an ASO (e.g., siRNA, shRNA, miRNA, or shmiRNA) sequence that binds to and inhibits the expression of the Grik2 mRNA, and a POMC promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an POMC promoter (e.g., POMC promoter having a nucleic acid sequence of SEQ ID NO: 720 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 720).

Alternatively, the lentiviral vector may include an ASO (e.g., siRNA, shRNA, miRNA, or shmiRNA) sequence that binds to and inhibits the expression of the Grik2 mRNA, and a PROX1 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an PROX1 promoter (e.g., PROX1 promoter having a nucleic acid sequence of SEQ ID NO: 721 or SEQ ID NO: 722 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 721 or SEQ ID NO: 722).

Alternatively, the lentiviral vector may include an ASO (e.g., siRNA, shRNA, miRNA, or shmiRNA) sequence that binds to and inhibits the expression of the Grik2 mRNA, and a MAP1 B promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an MAP1 B promoter (e.g., MAP1 B promoter having a nucleic acid sequence of any one of SEQ ID NOs: 723-725 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of any one of SEQ ID NOs: 723-725).

Alternatively, the lentiviral vector may include an ASO (e.g., siRNA, shRNA, miRNA, or shmiRNA) sequence that binds to and inhibits the expression of the Grik2 mRNA, and a Ta-1 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an Ta-1 promoter (e.g., Ta-1 promoter having a nucleic acid sequence of SEQ ID NO: 726 or SEQ ID NO: 727 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NO: 726 or SEQ ID NO: 727).

Alternatively, the lentiviral vector may include an ASO (e.g., siRNA, shRNA, miRNA, or shmiRNA) sequence that binds to and inhibits the expression of the Grik2 mRNA, and a U6 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an U6 promoter, such as a U6 promoter having a nucleic acid sequence of any one of SEQ ID NOs: 728-733 or 772, or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 728-733 or 772.

Alternatively, the lentiviral vector may include an ASO (e.g., siRNA, shRNA, miRNA, or shmiRNA) sequence that binds to and inhibits the expression of the Grik2 mRNA, and an H1 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an H1 promoter, such as an H1 promoter having a nucleic acid sequence of any one of SEQ ID NO: 734 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 734.

Alternatively, the lentiviral vector may include an ASO (e.g., siRNA, shRNA, miRNA, or shmiRNA) sequence that binds to and inhibits the expression of the Grik2 mRNA, and an 7SK promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an 7SK promoter, such as an 7SK promoter having a nucleic acid sequence of SEQ ID NO: 735 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 735.

Alternatively, the lentiviral vector may include an ASO (e.g., siRNA, shRNA, miRNA, or shmiRNA) sequence that binds to and inhibits the expression of the Grik2 mRNA, and an ApoE-hAAT promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an ApoE-hAAT promoter, such as an ApoE-hAAT promoter having a nucleic acid sequence of SEQ ID NO: 736 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 736.

Alternatively, the lentiviral vector may include an ASO (e.g., siRNA, shRNA, miRNA, or shmiRNA) sequence that binds to and inhibits the expression of the Grik2 mRNA, and a CAG promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an CAG promoter, such as a CAG promoter having a nucleic acid sequence of SEQ ID NO: 737 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 737.

Alternatively, the lentiviral vector may include an ASO (e.g., siRNA, shRNA, miRNA, or shmiRNA) sequence that binds to and inhibits the expression of the Grik2 mRNA, and a CBA promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and a CBA promoter, such as a CBA promoter having a nucleic acid sequence of SEQ ID NO: 738 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 738.

Alternatively, the lentiviral vector may include an ASO (e.g., siRNA, shRNA, miRNA, or shmiRNA) sequence that binds to and inhibits the expression of the Grik2 mRNA, and a CK8 promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and a CK8 promoter, such as CK8 promoter having a nucleic acid sequence of SEQ ID NO: 739 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 739.

Alternatively, the lentiviral vector may include an ASO (e.g., siRNA, shRNA, miRNA, or shmiRNA) sequence that binds to and inhibits the expression of the Grik2 mRNA, and an mU1a promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an mU1a promoter, such as an mU1 a promoter having a nucleic acid sequence of SEQ ID NO: 740 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 740.

Alternatively, the lentiviral vector may include an ASO (e.g., siRNA, shRNA, miRNA, or shmiRNA) sequence that binds to and inhibits the expression of the Grik2 mRNA, and an EF-1a promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and an EF-1a promoter, such as an EF-1a promoter having a nucleic acid sequence of SEQ ID NO: 741 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 741.

Alternatively, the lentiviral vector may include an ASO (e.g., siRNA, shRNA, miRNA, or shmiRNA) sequence that binds to and inhibits the expression of the Grik2 mRNA, and a TBG promoter. For example, the disclosed lentiviral vector may include a nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and a TBG promoter, such as TBG promoter having a nucleic acid sequence of SEQ ID NO: 742 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of SEQ ID NO: 742.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV1, HIV2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g., U.S. Pat. Nos. 6,013,516 and 5,994,136, both of which are incorporated herein by reference. In general, the vectors are plasmid-based or virus-based and are configured to carry the essential sequences for incorporating foreign nucleic acid and for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging proteins, namely gag, pol and env, as well as rev and tat is described in U.S. Pat. No. 5,994,136, incorporated herein by reference. This publication provides a first vector that can provide a nucleic acid encoding a viral gag and a pol gene and second vector that can provide a nucleic acid encoding a viral env to produce a packaging cell. Introducing a vector providing a heterologous gene into said packaging cell yields a producer cell which releases infectious viral particles carrying the foreign gene of interest. The env may be an amphotropic envelope protein which allows transduction of cells of human and other species. Typically, the nucleic acid molecule or the vector of the present disclosure include “control sequences,” which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

Viral Regulatory Elements

Viral regulatory elements are components of delivery vehicles used to introduce nucleic acid molecules into a host cell. Viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions. As other viral regulatory elements become known, these may be used with the methods and compositions described herein.

### Viral Vectors Encoding Grik2 Antisense Oligonucleotides

The present disclosure relates a nucleic acid vector for delivery of a heterologous polynucleotide, wherein the polynucleotide encodes an inhibitory ASO agent (e.g., siRNA, shRNA, miRNA, or shmiRNA, or shmiRNA) construct that specifically binds Grik2 mRNA and inhibits expression of GluK2 protein in a cell. Accordingly, an object of the disclosure provides a vector including an oligonucleotide sequence that is fully or substantially complementary to at least a region or portion of the Grik2 mRNA (e.g., any one of the regions or portions of a Grik2 mRNA selected from any one of SEQ ID NOs: 115-681, or variants thereof having at least 85% (e.g., at least 86%, 90%, 95%, 96%, 97%, 98%, 99%, or more) sequence identity to the nucleic acid sequence of any one of SEQ ID NOs: 115-681. The vector of the disclosure may include any variant of the oligonucleotide sequence that is fully or substantially complementary to one or more regions of the Grik2 mRNA. Additionally, the vector of the disclosure may include any variant of the oligonucleotide sequence is fully or substantially complementary to a Grik2 mRNA encoding any variant of the GluK2 protein.

Accordingly, the DNA encoding double stranded RNA of interest is incorporated into a gene cassette, e.g. an expression cassette in which transcription of the DNA is controlled by a promoter and/or other regulatory elements. The DNA is incorporated into such expression cassettes of the vector expressing a Grik2 ASO of interest (e.g., any one of SEQ ID NOs: 1-100) and are encapsidated by the viral vector of interest for delivery to target cells. The viral vectors of the disclosure thus encode any antisense RNA that hybridizes to any Grik2 mRNA transcript isoform (e.g., any one of SEQ ID NOs: 115-124). The viral vectors encode, e.g., any one of the siRNAs listed in Table 2 or Table 3.

Vectors of the disclosure deliver polynucleotides encoding an ASO that recognizes or binds to at least a portion or region of a Grik2 mRNA (e.g., any one of the regions or portions of Grik2 mRNA described in SEQ ID NOs: 115-681 or a variant thereof having at least 85% (e.g., at least 86%, 90%, 95%, 96%, 97%, 98%, 99%, or more) sequence identity to the nucleic acid sequence of any one of SEQ ID NOs: 115-681). The heterologous polynucleotide encoding the ASO agent may be part of a larger construct or scaffold that ensures the processing of such an ASO within a cell (e.g., a mammalian cell, such as, e.g., a human cell, such as, e.g., a neuronal cell, such as, e.g., a DGC). The polynucleotide encoding any one of the siRNAs listed in Table 2 or Table 3 may include a precursor or a portion of a microRNA gene (e.g., miR-30, miR-155, miR-281-1, or miR-124-3, among others), such as, e.g., a 5′ flanking sequence, a 3′ flanking sequence, or loop sequence of a microRNA gene.

Accordingly, an object of the disclosure relates to an expression vector including a heterologous polynucleotide and containing from 3 to 3, e.g., a promoter (e.g., any one of the promoters described in Table and Table 6), optionally an intron (e.g., any one of the introns described in Table 7), a nucleotide sequence encoding an ASO agent that inhibits Grik2 mRNA expression (e.g., ASO agent having a nucleic acid sequence of any one of SEQ ID NOs: 1-100 or a variant thereof having at least 85% (e.g., at least 86%, 90%, 95%, 96%, 97%, 98%, 99%, or more) sequence identity to the nucleic acid sequence of any one of SEQ ID NOs: 1-100), and a polyA sequence (e.g., any one of the polyA sequences described in Table 7). The expression vector may also include, from 5′ inverted terminal repeat (ITR) to 3′ ITR, a 5′ ITR (e.g., any one of the 5′ or 3′ ITR sequences described in Table 7), a promoter, optionally an intron, a nucleotide sequence encoding an ASO that inhibits Grik2 mRNA expression, a polyA sequence, and a 3′ ITR. The expression vector may further contain spacer and/or linker sequences adjoined to any of the foregoing vector elements.

In particular examples, the expression vector or polynucleotide may include a nucleotide sequence that encodes a stem and a loop which form a stem-loop structure, wherein the loop includes a nucleotide sequence encoding any one of the ASO agents listed in Table 2 or Table 3. For example, the expression vector or polynucleotide may include a nucleic acid sequence that encodes a loop region, wherein the loop region may be derived in whole or in part from wild type microRNA sequence gene (e.g., miR-30, miR-155, miR-281-1, or miR-124-3, among others) or be completely artificial. In a particular example, the loop region may be an miR-30a loop sequence. Furthermore, the stem-loop structure may include a guide sequence (e.g., an antisense RNA sequence, such as, e.g., any one of SEQ ID NOs: 1-100) and a passenger sequence that is complimentary to all or part of the guide sequence. For example, the passenger sequence may be complementary to all of the nucleotides of the guide sequence except for 10, 9, 8, 7, 6, 5, 4, 3, 2, or 1 nucleotide(s) of the guide sequence or the passenger sequence may be complementary to any one of SEQ ID NOs: 1-100.

Pre-miRNA or pri-miRNA scaffolds include guide (i.e., antisense) sequences of the disclosure. A pri-miRNA scaffold includes a pre-miRNA scaffold, and pri-miRNA may be 50-800 nucleotides in length (e.g., 50-800, 75-700, 100-600, 150-500, 200-400, or 250-300 nucleotides). In particular examples, the pre-mRNA may be 50-100 nucleotides (e.g., between 50-60, 60-70, 70-80, 80-90, or 90-100 nucleotides), 100-200 nucleotides (e.g., between 110-120, 120-130, 130-140, 140-150, 150-160, 160-170, 170-180, 180-190, or 190-200 nucleotides), 200-300 nucleotides(e.g., between 200-210, 210-220, 220-230, 230-240, 240-250, 250-260, 260-270, 270-280, 280-290, or 290-300 nucleotides), 300-400 nucleotides (e.g., between 300-310, 310-320, 320-330, 330-340, 340-350, 350-360, 360-370, 370-380, 380-390, or 390-400 nucleotides), 400-500 nucleotides (e.g., between 400-410, 410-420, 420-430, 430-440, 440-450, 450-460, 460-470, 470-480, 480-490, or 490-500 nucleotides), 500-600 nucleotides (e.g., between 500-510, 510-520, 520-530, 530-540, 540-550, 550-560, 560-570, 570-580, 580-590, or 590-600 nucleotides), 600-700 nucleotides (e.g., between 600-610, 610-620, 620-630, 630-640, 640-650, 650-660, 660-670, 670-680, 680-690, or 690-700 nucleotides), or 700-800 nucleotides (e.g., between 700-710, 710-720, 720-730, 730-740, 740-750, 750-760, 760-770, 770-780, 780-790, or 790-800 nucleotides). These engineered scaffolds allow processing of the pre-miRNA into a double stranded RNA comprising a guide strand and a passenger strand. As such, pre-miRNA includes a 5′ arm including the sequence encoding a guide (i.e., antisense) RNA, a loop sequence usually derived from a wild-type miRNA (e.g., miR-30, miR-155, miR-281-1, or miR-124-3, among others) and a 3′ arm including a sequence encoding a passenger (i.e., sense) strand which is fully or substantially complementary to the guide strand. Pre-miRNA “stem-loop” structures are generally longer than 50 nucleotides, e.g. 50-150 nucleotides (e.g., 50-60, 60-70, 70-80, 80-90, 90-100, 100-110, 110-120, 120-130, 130-140, or 140-150 nucleotides), 50-110 nucleotides (e.g., 50-60, 60-70, 70-80, 80-90, 90-100, 100-110 nucleotides), or 50-80 nucleotides (e.g., 50-60, 60-70, 70-80 nucleotides) in length. Pri-miRNA further includes 5′ flanking and 3′ flanking sequences, flanking the 5′ and 3′ arms, respectively. Flanking sequences are not necessarily contiguous with other sequences (the arm region or the guide sequence), are unstructured, unpaired regions, and may also be derived, in whole or in part, from one or more wild-type pri-miRNA scaffolds (e.g., pri-miRNA scaffolds derived, in whole or in part, from miR-30, miR-155, miR-281-1, or miR-124-3, among others). Flanking sequences are each at least 4 nucleotides in length, or up to 300 nucleotides or more in length (e.g., 4-300, 10-275, 20-250, 30-225, 40-200, 50-175, 60-150, 70-125, 80-100, or 90-95 nucleotides). Spacer sequences may be present as intervening between the aforementioned sequence structures, and in most instances provide linking polynucleotides, e.g., 1-30 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or nucleotides), to provide flexibility without interfering with functionality to the overall pre-miRNA structure. The spacer may be derived from a naturally occurring linking group from a naturally occurring RNA, a portion of a naturally occurring linking group, a poly-A or poly-U, or a random sequence of nucleotides, so long as the spacer does not interfere with the processing of the double stranded RNA, nor does the spacer interfere with the binding/interaction of the guide RNA with the target mRNA sequence.

According to the methods and compositions disclosed herein, the expression vector or polynucleotide including an nucleotide sequence may further encode (i) a 5′ stem-loop arm including a guide (e.g., antisense) strand and, optionally, a 5′ spacer sequence; and (ii) a 3′ stem-loop arm including a passenger (e.g., sense) strand and optionally a 3′ spacer sequence. In another example, the expression vector or polynucleotide including a nucleotide sequence may further encode (i) a 5′ stem-loop arm including a passenger strand and, optionally, a 5′ spacer sequence; and (ii) a 3′ stem-loop arm including a guide strand and optionally a 3′ spacer sequence. In another example, a uridine wobble base is present at the 5′ end of the guide strand. In a further example, the expression vector or polynucleotide includes a leading 5′ flanking region upstream of the guide sequence and the flanking region may be of any length and may be derived in whole or in part from wild type microRNA sequence, may be heterologous or derived from a miRNA of different origin from the other flanking regions or the loop, or may be completely artificial. A 3′ flanking region may mirror the 5′ flanking region in size and origin and the 3′ flanking region may be downstream (i.e., 3′) of the guide sequence. In yet another example, one or both of the 5′ flanking sequence and the 3′ flanking sequences are absent.

The expression vector or polynucleotide may include a nucleotide sequence that further encodes a first flanking region (e.g., any one of the 5′ flanking regions described in Table 8), said first flanking region includes a 5′ flanking sequence and, optionally, a 5′ spacer sequence. In a particular example, the first flanking region is located upstream (i.e., 5′) to said passenger strand. In another example, the expression vector or polynucleotide including a nucleotide sequence encodes a second flanking region (e.g., any one of the 3′ flanking regions described in Table 8), said second flanking region includes a 3′ flanking sequence and, optionally, a 3′ spacer sequence. In a particular example, the first flanking region is located 5′ to the guide strand.

According to the methods and compositions disclosed herein, the expression vector or polynucleotide may include a nucleotide sequence that encodes:
(a) a stem-loop sequence including, from 5′ to 3′:
   (i) a 5′ stem-loop arm including a guide nucleotide sequence which is at least 85% (e.g., at least 86%, 90%, 95%, 96%, 97%, 98%, 99%, or more) identical to any one of the ASO sequences listed in Table 2 or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
   (ii) a microRNA loop region, in which the loop region includes a microRNA loop sequence (e.g., a miR-30a, miR-155, miR-218-1, or miR-124-3 loop sequence (e.g., a microRNA loop sequence having a nucleic acid selected from any one of SEQ ID NOs: 758, 764, 767, or 770);
   (iii) a 3′ stem-loop arm including a passenger nucleotide sequence complementary or substantially complementary to the guide strand,
(b) a first flanking region located 5′ to the guide strand (e.g., any one of the 5′ flanking regions described in Table 8), in which the first flanking region includes a 5′ flanking sequence and, optionally, a 5′ spacer sequence; and
(c) a second flanking region (e.g., any one of the 3′ flanking regions described in Table 8) located 3′ to the passenger strand, in which the second flanking region includes a 3′ flanking sequence and, optionally, a 3′ spacer sequence.

In another example, the expression vector or polynucleotide includes a nucleotide sequence that encodes:
(a) a stem-loop sequence including, from 5′ to 3′ :
   (i) a 5′ stem-loop arm including a passenger nucleotide sequence which is complementary or substantially complementary to the guide nucleotide sequence;
   (ii) a microRNA loop region, in which the loop region includes a microRNA loop sequence (e.g., a miR-30a, miR-155, miR-218-1, or miR-124-3 loop sequence (e.g., a microRNA loop sequence having a nucleic acid selected from any one of SEQ ID NOs: 758, 764, 767, or 770);
   (iii) a 3′ stem-loop arm including a guide nucleotide sequence which is at least 85% (e.g., at least 86%, 90%, 95%, 96%, 97%, 98%, 99%, or more) identical to any one of the ASO sequences listed in Table 2 or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
(b) a first flanking region (e.g., any one of the 5′ flanking regions described in Table 8) located 5′ to the passenger strand; and
(c) a second flanking region (e.g., any one of the 3′ flanking regions described in Table 8) located 3′ to the guide strand, in which the second flanking region includes a 3′ flanking sequence and, optionally, a 3′ spacer sequence.

In another example, the expression vector or polynucleotide includes a nucleotide sequence that encodes:
(a) a stem-loop sequence including, from 5′ to 3′:
   (i) a 5′ stem-loop arm including a guide nucleotide sequence which is at least 85% (e.g., at least 86%, 90%, 95%, 96%, 97%, 98%, 99%, or more) identical to any one of the ASO sequences listed in Table 2 or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto);
   (ii) a microRNA loop region, in which the loop region includes a microRNA loop sequence (e.g., a miR-30a, miR-155, miR-218-1, or miR-124-3 loop sequence (e.g., a microRNA loop sequence having a nucleic acid selected from any one of SEQ ID NOs: 758, 764, 767, or 770);
   (iii) a 3′ stem-loop arm including a passenger nucleotide sequence which is complementary or substantially complementary to the guide nucleotide sequence;
(b) a 5′ flanking region (e.g., any one of the 5′ flanking regions described in Table 8) located 5′ to the guide strand; and
(c) a 3′ flanking region (e.g., any one of the 3′ flanking regions described in Table 8) located 3′ to the passenger strand, in which the second flanking region includes a 3′ flanking sequence and, optionally, a 3′ spacer sequence.

The length of the aforementioned guide strand and passenger strand may be between 19-50 (e.g., 19, 20, 21, 22, 23, 24, 25, 26-30, 31-35, 36-40, 41-45, or 46-50) nucleotides in length. In a particular example, the length of the guide strand is 19 nucleotides. In another example, the length of the guide strand is nucleotides. In another example, the length of the guide strand is 21 nucleotides. In another example, the length of the guide strand is 22 nucleotides. In another example, the length of the guide strand is 23 nucleotides. In another example, the length of the guide strand is 24 nucleotides. In another example, the length of the guide strand is nucleotides. In another example, the length of the guide strand is 26-nucleotides. In another example, the length of the guide strand is 31-nucleotides.

In another example, the length of the guide strand is 36-nucleotides. In another example, the length of the guide strand is 41-45 nucleotides. In another example, the length of the guide strand is 46-50 nucleotides. In a particular example, the length of the passenger strand is 19 nucleotides.

In another example, the length of the passenger strand is nucleotides. In another example, the length of the passenger strand is 21 nucleotides. In another example, the length of the passenger strand is 22 nucleotides. In another example, the length of the passenger strand is 23 nucleotides. In another example, the length of the passenger strand is 24 nucleotides. In another example, the length of the passenger strand is nucleotides. In another example, the length of the passenger strand is 26-nucleotides.

In another example, the length of the passenger strand is 31-nucleotides. In another example, the length of the passenger strand is 36-nucleotides. In another example, the length of the passenger strand is 41-45 nucleotides. In another example, the length of the passenger strand is 46-50 nucleotides.

The length of the guide and passenger sequence may vary based on the miRNA scaffold into which the guide and passenger strands are incorporated. When a given guide is adapted into a miRNA scaffold, the length of the guide can be extended to accommodate the natural structure and processing of a given miRNA scaffold. For example, guide sequences produced by the E-miR-scaffold are typically 22 nucleotides long. For most scaffolds, the guide sequences are extended at the 3′ end to be additionally complementary to the target mRNA sequence, but in some cases may involve modifying the 5′ start site of the guide, depending on the sequence of the miRNA scaffold.

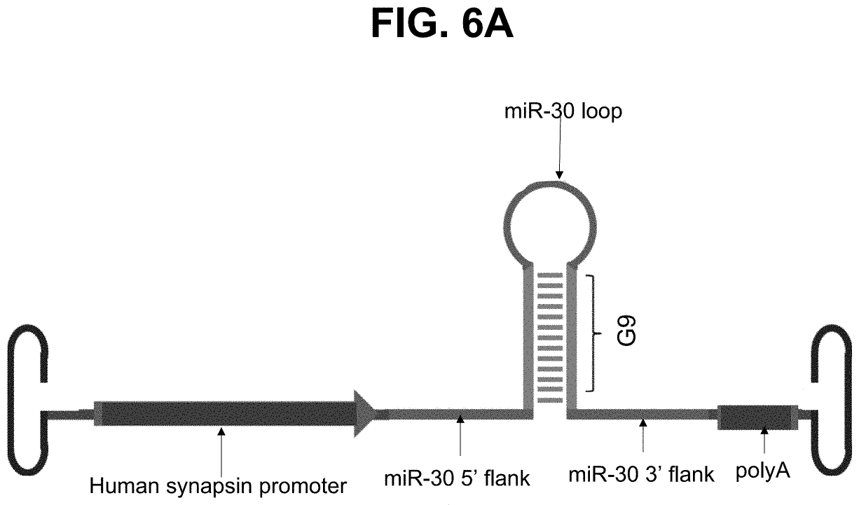

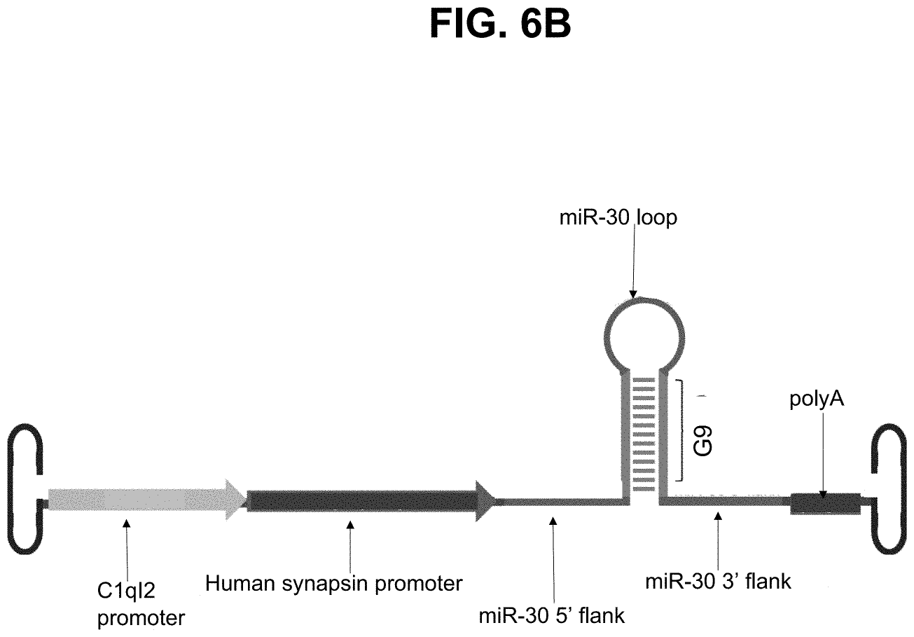

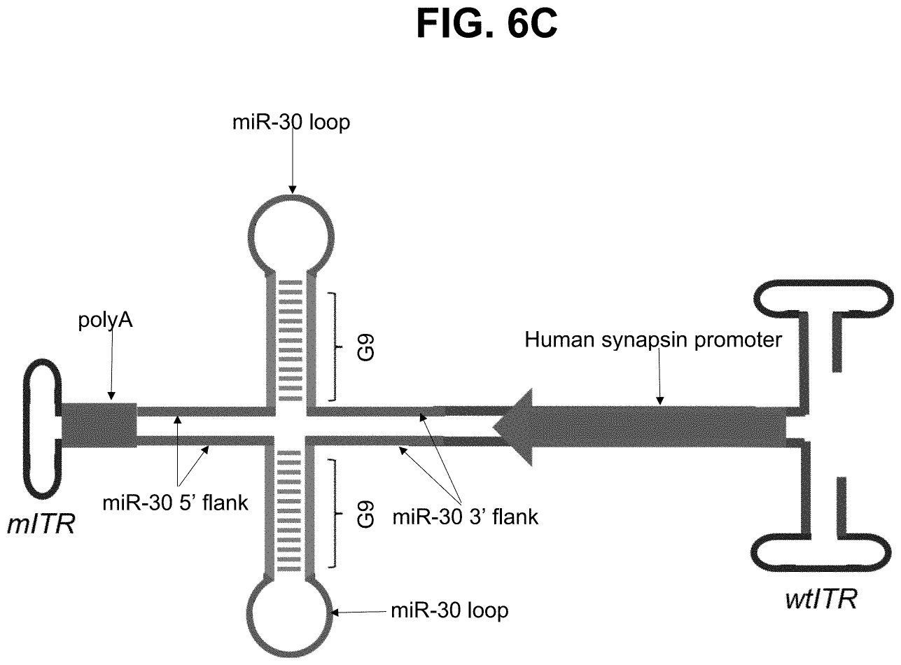

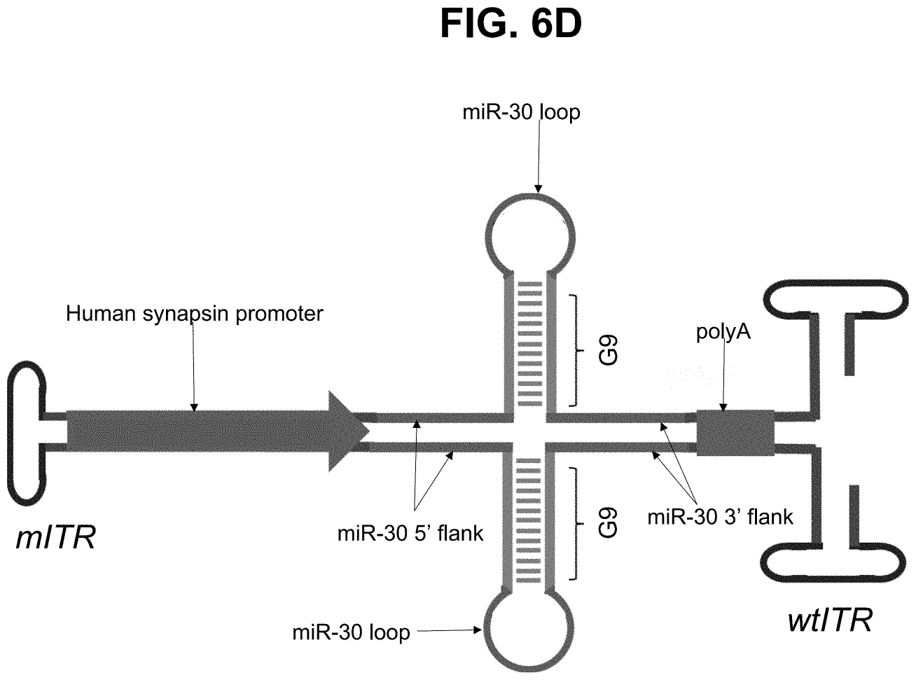

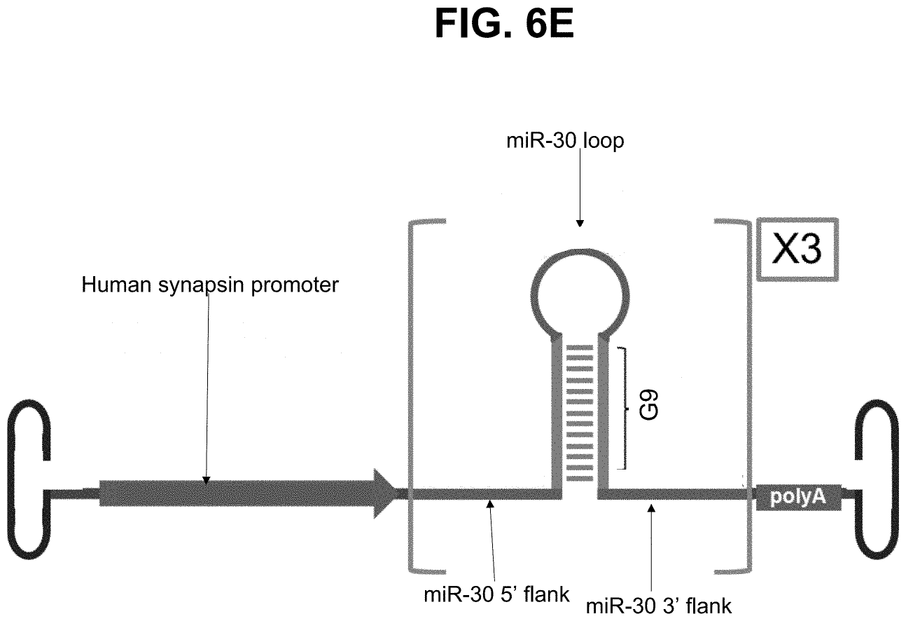

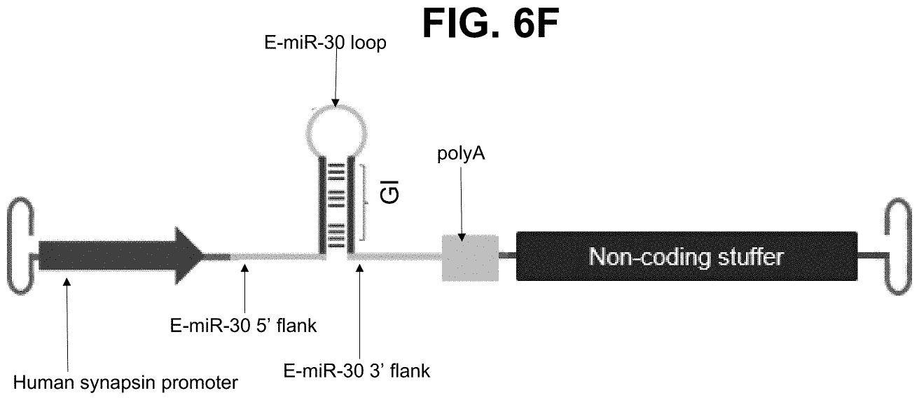

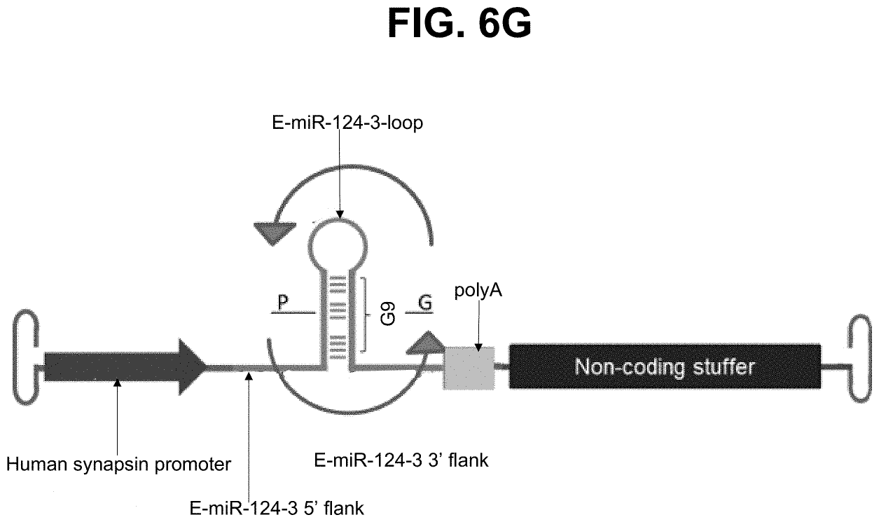

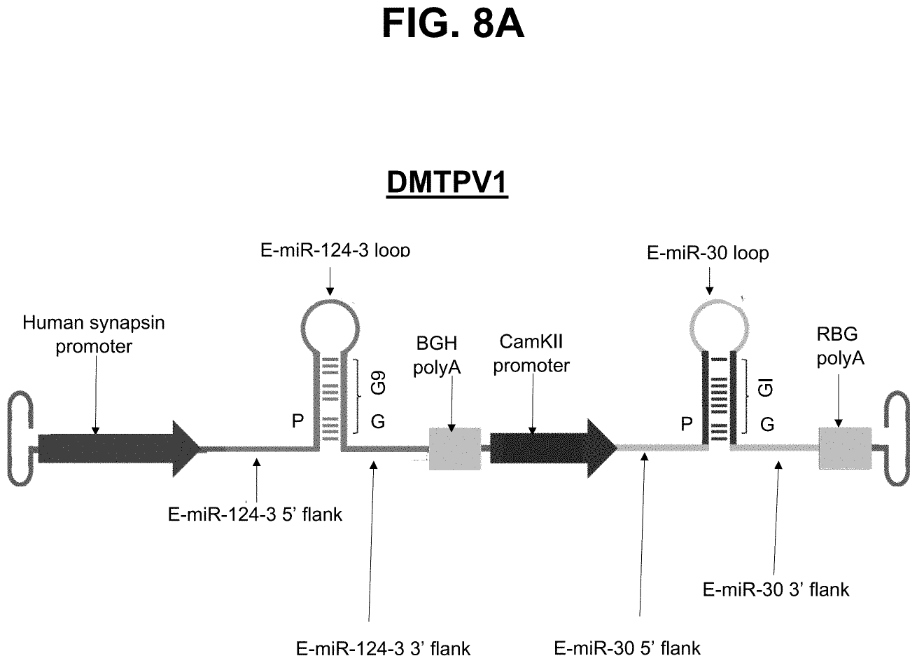

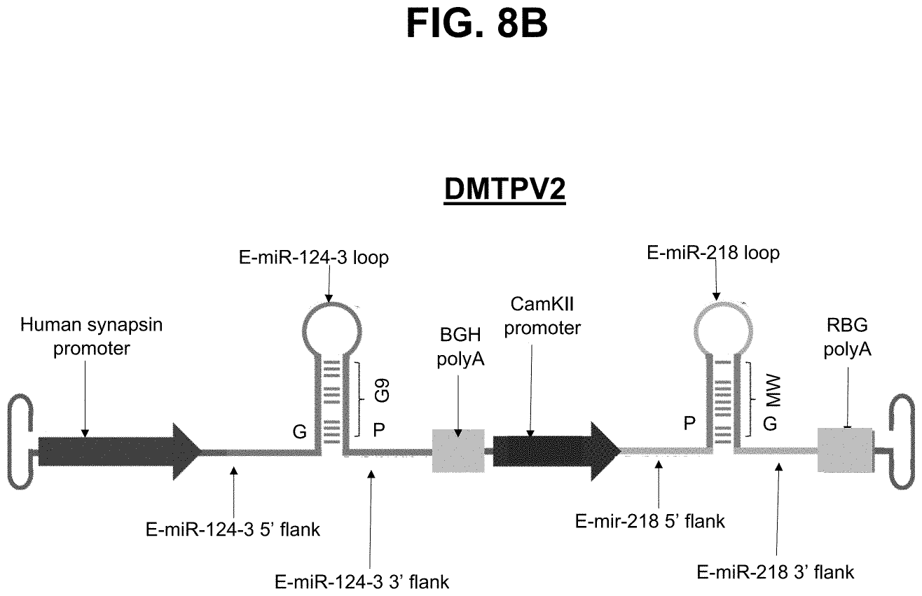

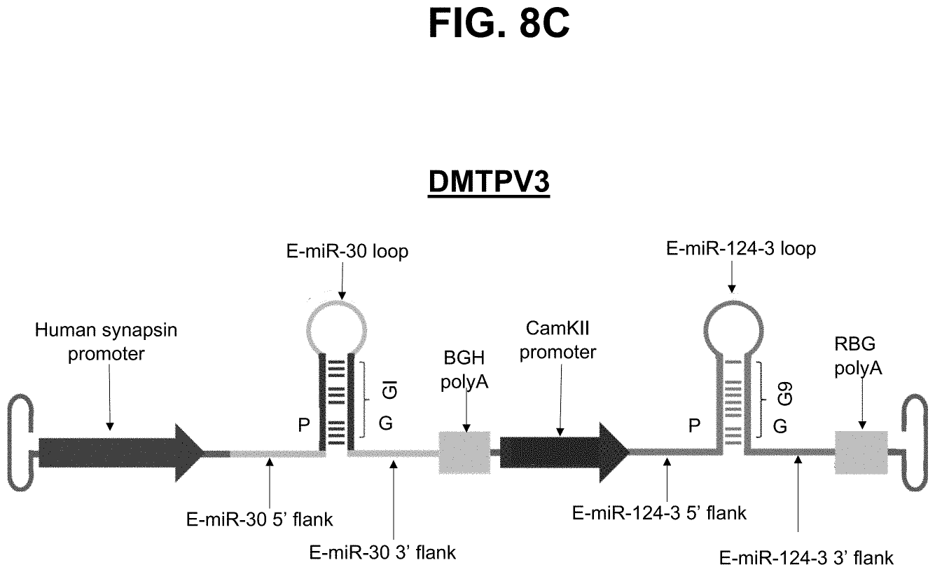

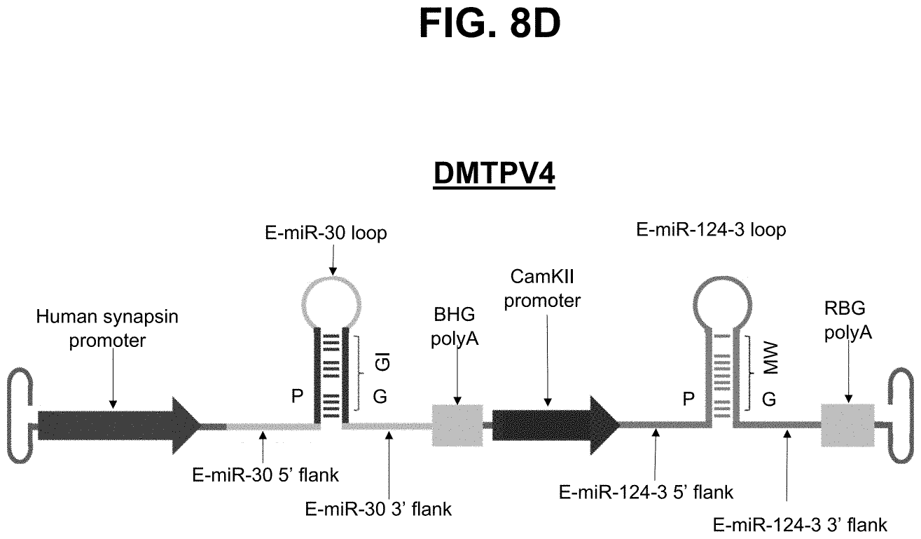

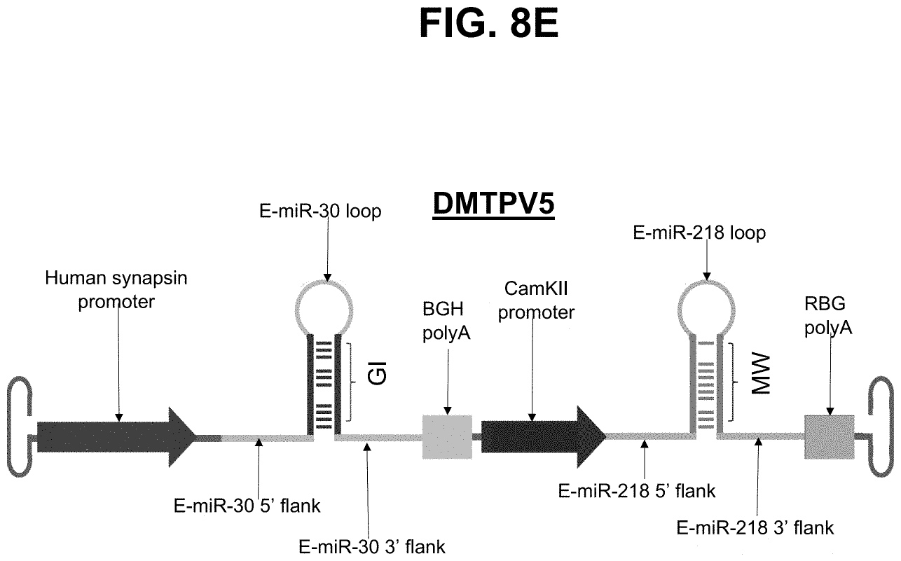

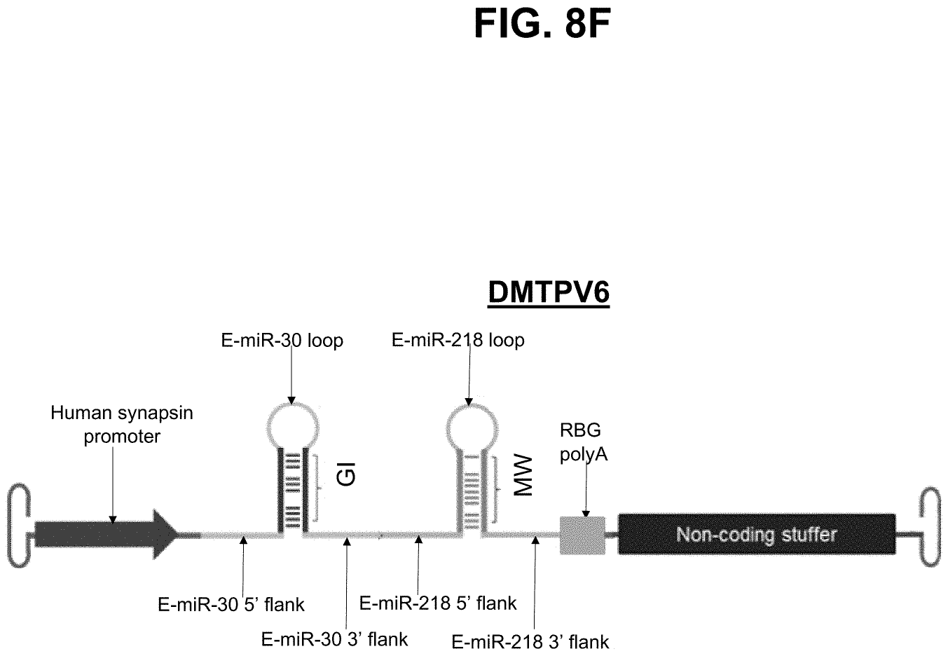

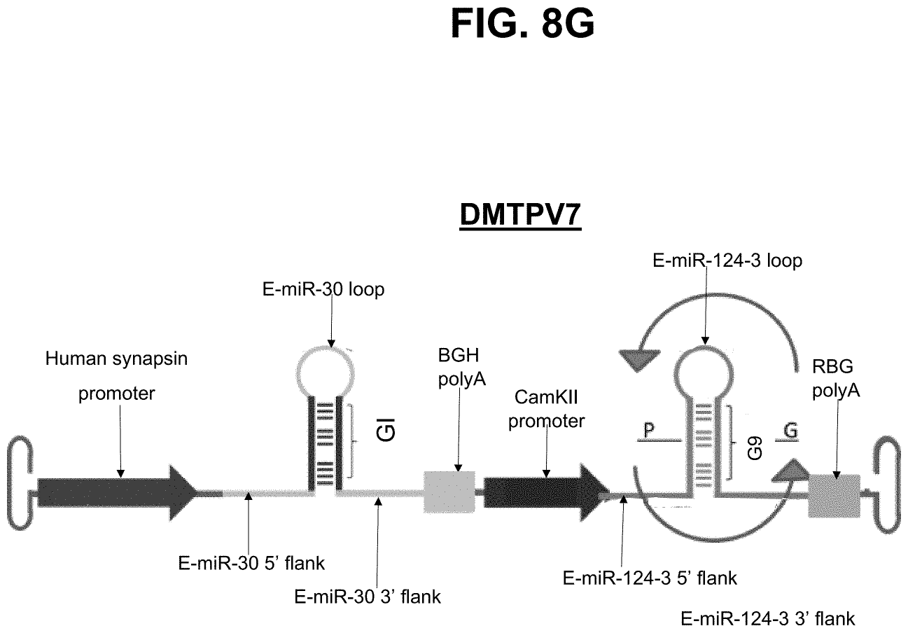

In certain cases, it may be desirable to modify miRNA guide and passenger strand expression levels and/or processing patterns to improve or modify targeting capacity of a given construct. As such, within a given miRNA framework/scaffold, the location of the guide and passenger strand may be exchanged (FIG. 6G); this may be in the context of a design including a stuffer sequence, or may be in the context of a design without a stuffer. This may additionally be in the context of a dual construct (as shown in FIG. 8G), or a concatenated construct (such as FIG. 8F). In order to accommodate this change, the sequence of the guide and/or passenger strand may be modified from the template “parental” design. Alternatively, modifications may be made to the guide and/or passenger strand sequence in order to affect changes in guide and passenger strand expression and/or processing patterns.

In a particular example, the vector or polynucleotide includes a miR-30a sequence, in which the first flanking region includes a nucleotide sequence at least 90% (e.g., at least 90%, 91%, 92%, 93%, 94%, 95%, 96%, 97%, 98%, 99%, or more) identical to any one of SEQ ID NOs: 752, 754, 756, and 759 (see Table 8).

In some embodiments, the vector or polynucleotide includes a miR-30a sequence, in which the second flanking region includes a nucleotide sequence at least 90% (e.g., at least 90%, 91%, 92%, 93%, 94%, 95%, 96%, 97%, 98%, 99%, or more) identical to any one of SEQ ID NOs: 753, 755, 757, and 760 (see Table 8).

In another example, the vector or polynucleotide includes a miR-30a structure in which the loop region includes the nucleotide sequence of SEQ ID NO: 758 or SEQ ID NO: 761, or a sequence at least 90% (e.g., at least 90%, 91%, 92%, 93%, 94%, 95%, 96%, 97%, 98%, 99%, or more) identical to SEQ ID NO: 758 or SEQ ID NO: 761 (see Table 8).

In a particular example, the vector or polynucleotide includes a miR-155 sequence, in which the first flanking region includes a nucleotide sequence at least 90% (e.g., at least 90%, 91%, 92%, 93%, 94%, 95%, 96%, 97%, 98%, 99%, or more) identical to SEQ ID NO: 762 (see Table 8).

In some embodiments, the vector or polynucleotide includes a miR-155 sequence, in which the second flanking region includes a nucleotide sequence at least 90% (e.g., at least 90%, 91%, 92%, 93%, 94%, 95%, 96%, 97%, 98%, 99%, or more) identical to SEQ ID NO: 763 (see Table 6).

In another example, the vector or polynucleotide includes a miR-155 structure in which the loop region includes the nucleotide sequence of SEQ ID NO: 764, or a sequence at least 90% (e.g., at least 90%, 91%, 92%, 93%, 94%, 95%, 96%, 97%, 98%, 99%, or more) identical to SEQ ID NO: 764 (see Table 8).

In a particular example, the vector or polynucleotide includes a miR-218-1 sequence, in which the first flanking region includes a nucleotide sequence at least 90% (e.g., at least 90%, 91%, 92%, 93%, 94%, 95%, 96%, 97%, 98%, 99%, or more) identical to SEQ ID NO: 765 (see Table 8).

In some embodiments, the vector or polynucleotide includes a miR-218-1 sequence, in which the second flanking region includes a nucleotide sequence at least 90% (e.g., at least 90%, 91%, 92%, 93%, 94%, 95%, 96%, 97%, 98%, 99%, or more) identical to SEQ ID NO: 766 (see Table 8).

In another example, the vector or polynucleotide includes a miR-218-1 structure in which the loop region includes the nucleotide sequence of SEQ ID NO: 767, or a sequence at least 90% (e.g., at least 90%, 91%, 92%, 93%, 94%, 95%, 96%, 97%, 98%, 99%, or more) identical to SEQ ID NO: 767 (see Table 8).

In a particular example, the vector or polynucleotide includes a miR-124-3 sequence, in which the first flanking region includes a nucleotide sequence at least 90% (e.g., at least 90%, 91%, 92%, 93%, 94%, 95%, 96%, 97%, 98%, 99%, or more) identical to SEQ ID NO: 768 (see Table 8).

In some embodiments, the vector or polynucleotide includes a miR-124-3 sequence, in which the second flanking region includes a nucleotide sequence at least 90% (e.g., at least 90%, 91%, 92%, 93%, 94%, 95%, 96%, 97%, 98%, 99%, or more) identical to SEQ ID NO: 769 (see Table 8).

In another example, the vector or polynucleotide includes a miR-124-3 structure in which the loop region includes the nucleotide sequence of SEQ ID NO: 770, or a sequence at least 90% (e.g., at least 90%, 91%, 92%, 93%, 94%, 95%, 96%, 97%, 98%, 99%, or more) identical to SEQ ID NO: 770 (see Table 8).

The expression vector may be a plasmid and may include, e.g., one or more of an intron sequence (e.g., an intron sequence of SEQ ID NO: 743 or SEQ ID NO: 744 or a variant thereof having at least 85% (e.g., at least 85% 90%, 91%, 92%, 93%, 94%, 95%, 96%, 97%, 98%, 99%, or more) sequence identity to the nucleic acid sequence of SEQ ID NO: 743 or SEQ ID NO: 744), a linker sequence, or a stuffer sequence.

TABLE 8
MicroRNA Sequences

| | SEQ ID | |
|---|---|---|
| Description | NO | Nucleotide sequence |
| A-hsa-miR- | 752 | GTTTGAATGAGGCTTCAGTACTTTACAGAATCGTTGCCTGCACATCT |
| 30a | | TGGAAACACTTGCTGGGATTACTTCTTCAGGTTAACCCAACAGAAGG |
| 5′ flanking | | CTCGAGAAGGTATATTGCTGTTGACAGTGAGCGC |
| region #1 | | |
| A-hsa-miR- | 753 | TTGCCTACTGCCTCGGAATTCAAGGGGCTACTTTAGGAGCAATTATC |
| 30a | | TTGTTTACTAAAACTGAATACCTTGCTATCTCTTTGATACATTTTTACA |
| 3′ flanking | | AAGCTGAATTAAAATGGTATAAATTA |
| region #1 | | |
| A-hsa-miR- | 754 | GTTTGAATGAGGCTTCAGTACTTTACAGAATCGTTGCCTGCACATCT |
| 30a | | TGGAAACACTTGCTGGGATTACTTCTTCAGGTTAACCCAACAGAAGG |
| 5′ flanking | | CTCGAGAAGGTATATTGCTGTTGACAGTGAGCGAC |
| region #2 | | |
| A-hsa-miR- | 755 | GCTGCCTACTGCCTCGGAATTCAAGGGGCTACTTTAGGAGCAATTAT |
| 30a | | CTTGTTTACTAAAACTGAATACCTTGCTATCTCTTTGATACATTTTTAC |
| 3′ flanking | | AAAGCTGAATTAAAATGGTATAAATTA |
| region #2 | | |
| A-hsa-miR- | 756 | GTTTGAATGAGGCTTCAGTACTTTACAGAATCGTTGCCTGCACATCT |
| 30a | | TGGAAACACTTGCTGGGATTACTTCTTCAGGTTAACCCAACAGAAGG |
| 5′ flanking | | CTCGAGAAGGTATATTGCTGTTGACAGTGAGCGA |
| region #3 | | |
| A-hsa-miR- | 757 | TGCCTACTGCCTCGGAATTCAAGGGGCTACTTTAGGAGCAATTATCT |
| 30a | | TGTTTACTAAAACTGAATACCTTGCTATCTCTTTGATACATTTTTACAA |
| 3′ flanking | | AGCTGAATTAAAATGGTATAAATTA |
| region #3 | | |
| A-hsa-miR- | 758 | TAGTGAAGCCACAGATG |
| 30a loop | | |
| region | | |
| E-hsa-miR- | 759 | GTTTGAATGAGGCTTCAGTACTTTACAGAATCGTTGCCTGCACATCT |
| 30a | | TGGAAACACTTGCTGGGATTACTTCTTCAGGTTAACCCAACAGAAGG |
| 5′ flanking | | CTAAAGAAGGTATATTGCTGTTGACAGTGAGCGAC |
| region | | |
| E-hsa-miR- | 760 | GCTGCCTACTGCCTCGGACTTCAAGGGGCTACTTTAGGAGCAATTA |
| 30a | | TCTTGTTTACTAAAACTGAATACCTTGCTATCTCTTTGATACATTTTTA |
| 3′ flanking | | CAAAGCTGAATTAAAATGGTATAAATTA |
| region | | |
| E-hsa-miR- | 761 | CTGTGAAGCCACAGATGGG |
| 30a | | |
| loop region | | |
| E-hsa-miR- | 762 | CAAACCAGGAAGGGGAAATCTGTGGTTTAAATTCTTTATGCCTCATC |
| 155 | | CTCTGAGTGCTGAAGGCTTGCTGTAGGCTGTATGCTG |
| 5′ flanking | | |
| region | | |
| E-hsa-miR- | 763 | CAGTGTATGATGCCTGTTACTAGCATTCACATGGAACAAATTGCTGC |
| 155 | | CGTGGGAGGATGACAAAGAAGCATGAGTCACCCTGCTGGATAAACT |
| 3′ flanking | | TAGACTTCAGGCTTTATCATTTTTCAATCTGTTAATCATAATCTGGTC |
| region | | ACTGGGATGTTCAACCTTAAACTAAGTTTTGAAAGTAAGGTTATTTAA |
| | | AAGATTTATCAGTA |
| E-hsa-miR- | 764 | TTTGCCTCCAACT |
| 155 | | |
| loop region | | |
| E-hsa-miR- | 765 | ACGTTTCCAGAACGTCTGTAGCTTTTCTCCTCCTTCCCTCCATTTTCC |
| 218-1 | | TCTTGGTCTTACCTTTGGCCTAGTGGTTGGTGTAGTGATAATGTAGC |
| 5′ flanking | | GAGATTTTCTG |
| region | | |
| E-hsa-miR- | 766 | TGGAACGTCACGCAGCTTTCTACAGCATGACAAGCTGCTGAGGCTT |
| 218-1 | | AAATCAGGATTTTCCTGTCTCTTTCTACAAAATCAAAATGAAAAAAGA |
| 3′ flanking | | GGGCTTTTTAGGCATCTCCGAGATTATGTG |
| region | | |
| E-hsa-miR- | 767 | GGTTGCGAGGTATGAGTAAA |
| 218-1 | | |
| loop region | | |
| E-hsa-miR- | 768 | TCTGCCGCGGAAAGGGGAGAAGTGTGGGCTCCTCCGAGTCGGGGG |
| 124-3 | | CGGACTGGGACAGCACAGTCGGCTGAGCGCAGCGCCCCCGCCCTG |
| 5′ flanking | | CCCGCCACGCGGCGAAGACGCCTGAGCGTTCGCGCCCCTCGGGC |
| region | | GAGGACCCCACGCAAGCCCGAGCCGGTCCCGACCCTGGCCCCGAC |
| | | GCTCGCCGCCCGCCCCAGCCCTGAGGGCCCCTC |
| E-hsa-miR- | 769 | GAGAGGCGCCTCCGCCGCTCCTTTCTCATGGAAATGGCCCGCGAG |
| 124-3 | | CCCGTCCGGCCCAGCGCCCCTCCCGCGGGAGGAAGGCGAGCCCG |
| 3′ flanking | | GCCCCCGGCGGCCATTCGCGCCGCGGACAAATCCGGCGAACAATG |
| region | | CGCCCGCCCAGAGTGCGGCCCAGCTGCCGGGCCGGGGATCTGGC |
| | | CGCGGGACACAAAGGGGCCCGCACGCCTCTGGCGT |
| E-hsa-miR- | 770 | ATTTAATGTCTATACAAT |
| 124-3 | | |
| loop region | | |

Accordingly, an object of the disclosure relates to a vector including polynucleotide having the nucleic acid sequence of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100. For example, the vector may include a polynucleotide having at least 90% (e.g., at least 90%, 95%, 96%, 97%, 98%, 99%, or more) sequence identity to the nucleic acid sequence of any one of SEQ ID NOs: 1-100. In another example, the vector may include a polynucleotide having least 95% (e.g., at least 95%, 96%, 97%, 98%, 99%, or more) sequence identity to the nucleic acid sequence of any one of SEQ ID NOs: 1-100. The vector of the disclosure may further include a polynucleotide having the nucleic acid sequence of any one of SEQ ID NOs: 1-100.

In particular, the vector may include the sequence of any one of SEQ ID NOs: 1-100 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of any one of SEQ ID NOs: 1-100 and a promoter (e.g., any one of the promoters listed in Table or Table 6, or a).

The variants discussed above may include, for instance, naturally-occurring variants due to allelic variations between individuals (e.g., polymorphisms), alternative splicing forms, etc. The term variant also includes genes sequences of the disclosure from other sources or organisms. Variants may be substantially homologous to sequences according to the disclosure. Variants of the genes of the disclosure also include nucleic acid sequences, which hybridize to a sequence as defined above (or a complementary strand thereof) under stringent hybridization conditions. Typical stringent hybridization conditions include temperatures above 30° C., above 35° C., or in excess of 42° C., and/or salinity of less than about 500 mM or less than 200 mM. Hybridization conditions may be adjusted by, e.g., modifying the temperature, salinity and/or the concentration of other reagents such as SDS, SSC, etc.

The present disclosure further provides non-viral vectors (e.g., a plasmid containing a polynucleotide encoding a Grik2-targeting ASO agent disclosed herein) for the delivery of heterologous polynucleotides to target cells of interest. In other cases, the viral vector of the disclosure may be an AAV vector adenoviral, a retroviral, a lentiviral, or a herpesvirus vector.

One or more expression cassettes may be employed. Each expression cassette may include at least a promoter sequence (e.g., a neuronal cell promoter) operably linked to a sequence encoding the RNA of interest. Each expression cassette may consist of additional regulatory elements, spacers, introns, UTRs, polyadenylation site, and the like. The expression cassette can be multigene with respect to the polynucleotides encoding e.g. two or more ASO agents. The expression cassette may further include a promoter, a nucleic acid encoding one or more ASO agents of interest, and a polyA sequence.

In a particular example, the expression cassette includes 5′ - promoter sequence, a polynucleotide sequence encoding a first ASO agent of interest (e.g., any one of SEQ ID NOs: 1-100), a sequence encoding a second ASO agent of interest (e.g., any one of SEQ ID NOs: 1-100), and a polyA sequence-3′.

The viral vector may further include a nucleic acid sequence encoding an antibiotic resistance gene such as the genes of resistance AmpR, kanamycin, hygromycin B, geneticin, blasticidin S, gentamycin, carbenicillin, chloramphenicol, nourseothricin, or puromycin.

Exemplary Expression Cassettes

The present disclosure provides expression cassettes that, when incorporated into an expression vector (e.g., a plasmid or viral vector (e.g., AAV or lentiviral vector)), promote the expression of a heterologous polynucleotide encoding an ASO agent (e.g., ASO agent having a nucleic acid sequence of any one of SEQ ID NOs: 1-100) that hybridizes to and inhibits the expression of a Grik2 mRNA.

Generally, an expression cassette incorporated into a nucleic acid vector will include a heterologous polynucleotide containing a heterologous gene regulatory sequence (e.g., a promoter (e.g., any one of the promoters described in Table or Table 6) and, optionally, an enhancer sequence (e.g., an enhancer sequence described in Table 7)), a 5′ flanking sequence (e.g., any one of SEQ ID NOs: 752, 754, 756, 759, 762, 765, or 768), a stem-loop sequence containing a stem-loop5′ arm, a loop sequence (e.g., any one of SEQ ID NOs: 758, 761, 764, 767, or 770), a stem-loop3′ arm, a 3′ flanking sequence (e.g., any one of SEQ ID NOs: 753, 755, 757, 760, 763, 766, or 769), optionally, a Woodchuck Hepatitis Posttranscriptional Regulatory Element (WRPE), and a polyA sequence (e.g., SEQ ID NO: 750, 751, 792, or 793). In the case of an AAV vector, the expression cassette may be flanked on its 5′ and 3′ ends by a 5′ ITR and a 3′ ITR sequence (e.g., any one of the 5′ or 3′ ITR sequences described in Table 7), respectively. Typically, AAV2 ITR sequences are contemplated for use in conjunction with the methods and compositions disclosed herein, however, ITR sequences from other AAV serotypes disclosed herein may also be employed (see section “AAV Vectors” above). Without limiting the scope of the present disclosure and solely to exemplify expression cassettes suitable for use with the disclosed methods and composition, Table 9 and Table 10, which are incorporated by reference herein in their entirety from U.S. Provisional Patent Application No.: 63/050,742, feature exemplary expression cassette constructs with expression cassette elements moving from the 5′ to the 3′ direction useful for inducing transgene expression in neurons or ubiquitously, respectively. The general architecture of the construct includes at least the following elements oriented in a 5′ to 3′ direction:
(i) A 5′ ITR sequence (for AAV vectors only; e.g., SEQ ID NO: 746 or SEQ ID NO: 747);
(ii) a promoter sequence (e.g., any one of the promoter sequences listed in Table or Table 6);
(iii) a 5′ flanking sequence (e.g., any one of SEQ ID NOs: 752, 754, 756, 759, 762, 765, or 768);
(iv) a stem-loop sequence that includes in the 5′ to 3′ direction:
   a. a stem-loop5′ arm, in which the stem-loop5′ arm includes a guide sequence containing at least an ASO sequence of any one of SEQ ID NOs: 1-100 or passenger sequence that is complementary or substantially complementary (e.g., containing no more than 10, no more than 9, no more than 8, no more than 7, no more than 6, 5, no more than 4, no more than 3, no more than 2, or no more than 1 mismatched nucleotides), to the ASO sequence of any one of SEQ ID NOs: 1-100;
   b. a loop sequence (e.g., a miR-30, miR-155, miR-218-1, or miR-124-3 loop sequence, such as, e.g., a loop sequence of any one of SEQ ID NOs: 758, 761, 764, 767, or 770); and
   c. a stem-loop3′ arm, in which the stem-loop3′ arm includes a guide sequence containing at least an ASO sequence of any one of SEQ ID NOs: 1-100 or a passenger sequence that is substantially complementary (e.g., containing no more than 10, no more than 9, no more than 8, no more than 7, no more than 6, 5, no more than 4, no more than 3, no more than 2, or no more than 1 mismatched nucleotides) to the ASO sequence of any one of SEQ ID NOs: 1-100;
(v) a 3′ flanking sequence (e.g., any one of SEQ ID NOs: 753, 755, 757, 760, 763, 766, or 769);
(vi) optionally, a WPRE sequence;
(vii) a polyA sequence (e.g., SEQ ID NO: 750, SEQ ID NO: 751, SEQ ID NO: 792, or SEQ ID NO: 793); and
(viii) a 3′ ITR sequence (for AAV vectors only; e.g., SEQ ID NO: 748, SEQ ID 0: 749, or SEQ ID NO: 789).

In a particular example, the disclosure provides an expression cassette including a hSyn promoter (e.g., any one of SEQ ID NOs: 682-685 and 790) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another example, the disclosure provides an expression cassette including a CaMKII promoter (e.g., any one of SEQ ID NOs: 687-691 and 802) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another example, the disclosure provides an expression cassette including a CAG promoter (e.g., SEQ ID NO: 737) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another example, the disclosure provides an expression cassette including a CBA promoter (e.g., SEQ ID NO: 738) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 25 99%, or more) sequence identity to the corresponding target sequence described in Table 4 or any one of SEQ ID NOs: 164-681, and a passenger sequence that is fully or substantially complementary to the guide sequence.

In another example, the disclosure provides an expression cassette including a U6 promoter (e.g., any one of SEQ ID NOs: 728-733) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another example, the disclosure provides an expression cassette including a H1 promoter (e.g., SEQ ID NO: 734) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another example, the disclosure provides an expression cassette including a 7SK promoter (e.g., SEQ ID NO: 735) operably linked to a polynucleotide including an anti-Grik2 guide sequence that is fully or substantially complementary to a Grik2 mRNA target sequence selected from the group consisting of target sequences described in Table 4 or any one of SEQ ID NOs: 164-681, or a variant thereof having at least 85% (at least 85%, 86%, 87%, 88%, 89%, 90%, 91%, 92%, 93%, 94%, 95%, 96%, 97%, 98%, 99%, or more) sequence identity to the corresponding target sequence described in Table 4 or any one of SEQ ID NOs: 164-681, and a passenger sequence that is fully or substantially complementary to the guide sequence.

In another example, the disclosure provides an expression cassette including a hSyn promoter (e.g., any one of SEQ ID NOs: 682-685 and 790) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another example, the disclosure provides an expression cassette including a CaMKII promoter (e.g., any one of SEQ ID NOs: 687-691 and 802) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another example, the disclosure provides an expression cassette including a CAG promoter (e.g., SEQ ID NO: 737) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another example, the disclosure provides an expression cassette including a CBA promoter (e.g., SEQ ID NO: 738) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another example, the disclosure provides an expression cassette including a U6 promoter (e.g., any one of SEQ ID NOs: 728-733) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another example, the disclosure provides an expression cassette including a H1 promoter (e.g., SEQ ID NO: 734) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another example, the disclosure provides an expression cassette including a 7SK promoter (e.g., SEQ ID NO: 735) operably linked to a polynucleotide including an anti-Grik2 guide sequence selected from the group consisting of any one of SEQ ID NOs: 1-100 or a variant thereof having at least 85% (at least 85%, 86%, 87%, 88%, 89%, 90%, 91%, 92%, 93%, 94%, 95%, 96%, 97%, 98%, 99%, or more) sequence identity to any one of SEQ ID NOs: 1-100, and a passenger sequence that is fully or substantially complementary to the guide sequence.

In another example, the disclosure provides an expression cassette selected from any one of the expression cassettes described in Table 9 below.

TABLE 9
Exemplary expression cassettes
5′ ------------------------------------------------------------------------------------------------------→ 3′

| AAV | | | | | | | | AAV |
|---|---|---|---|---|---|---|---|---|
| 5′ | | 5′ | | | | 3′ | 3′ | 3′ |
| ITR | Promoter | flank | 5p stem-loop arm | Loop | 3p stem-loop arm | flank | UTR | ITR |
| A | hSyn (e.g., | B | Guide sequence | C | Passenger | D | E | F |
| | any one of | | (e.g., any one of | | sequence (e.g. | | | |
| | SEQ ID NOs: | | SEQ ID NOs: | | sequence fully or | | | |
| | 682-685 | | 1-100) | | substantially | | | |
| | and 790) | | | | complementary to | | | |
| | | | | | the guide sequence) | | | |
| A | CaMKII (e.g., | B | Guide sequence | C | Passenger | D | E | F |
| | any one of | | (e.g., any one of | | sequence (e.g. | | | |
| | SEQ ID NOs: | | SEQ ID NOs: | | sequence fully or | | | |
| | 687-691 | | 1-100) | | substantially | | | |
| | and 802) | | | | complementary to | | | |
| | | | | | the guide sequence) | | | |
| A | CAG (e.g., | B | Guide sequence | C | Passenger | D | E | F |
| | SEQ ID NO: | | (e.g., any one of | | sequence (e.g. | | | |
| | 737) | | SEQ ID NOs: | | sequence fully or | | | |
| | | | 1-100) | | substantially | | | |
| | | | | | complementary to | | | |
| | | | | | the guide sequence) | | | |
| A | CBA (e.g., | B | Guide sequence | C | Passenger | D | E | F |
| | SEQ ID NO: | | (e.g., any one of | | sequence (e.g. | | | |
| | 738) | | SEQ ID NOs: | | sequence fully or | | | |
| | | | 1-100) | | substantially | | | |
| | | | | | complementary to | | | |
| | | | | | the guide sequence) | | | |
| A | U6 (e.g., | B | Guide sequence | C | Passenger | D | E | F |
| | any one of | | (e.g., any one of | | sequence (e.g. | | | |
| | SEQ ID NOs: | | SEQ ID NOs: | | sequence fully or | | | |
| | 728-733) | | 1-100) | | substantially | | | |
| | | | | | complementary to | | | |
| | | | | | the guide sequence) | | | |
| A | H1 (e.g., | B | Guide sequence | C | Passenger | D | E | F |
| | SEQ ID NO: | | (e.g., any one of | | sequence (e.g. | | | |
| | 734) | | SEQ ID NOs: | | sequence fully or | | | |
| | | | 1-100) | | substantially | | | |
| | | | | | complementary to | | | |
| | | | | | the guide sequence) | | | |
| A | 7SK (e.g., | B | Guide sequence | C | Passenger | D | E | F |
| | SEQ ID NO: | | (e.g., any one of | | sequence (e.g. | | | |
| | 735) | | SEQ ID NOs: | | sequence fully or | | | |
| | | | 1-100) | | substantially | | | |
| | | | | | complementary to | | | |
| | | | | | the guide sequence) | | | |
| A | hSyn (e.g., | B | Passenger | C | Guide sequence | D | E | F |
| | any one of | | sequence (e.g. | | (e.g., any one of | | | |
| | SEQ ID NOs: | | sequence fully or | | SEQ ID NOs: | | | |
| | 682-685 | | substantially | | 1-100) | | | |
| | and 790) | | complementary to | | | | | |
| | | | the guide sequence) | | | | | |
| A | CaMKII (e.g., | B | Passenger | C | Guide sequence | D | E | F |
| | any one of | | sequence (e.g. | | (e.g., any one of | | | |
| | SEQ ID NOs: | | sequence fully or | | SEQ ID NOs: | | | |
| | 687-691 | | substantially | | 1-100) | | | |
| | and 802) | | complementary to | | | | | |
| | | | the guide sequence) | | | | | |
| A | CAG (e.g., | B | Passenger | C | Guide sequence | D | E | F |
| | SEQ ID NO: | | sequence (e.g. | | (e.g., any one of | | | |
| | 737) | | sequence fully or | | SEQ ID NOs: | | | |
| | | | substantially | | 1-100) | | | |
| | | | complementary to | | | | | |
| | | | the guide sequence) | | | | | |
| A | CBA (e.g., | B | Passenger | C | Guide sequence | D | E | F |
| | SEQ ID NO: | | sequence (e.g. | | (e.g., any one of | | | |
| | 738) | | sequence fully or | | SEQ ID NOs: | | | |
| | | | substantially | | 1-100) | | | |
| | | | complementary to | | | | | |
| | | | the guide sequence) | | | | | |
| A | U6 (e.g., | B | Passenger | C | Guide sequence | D | E | F |
| | any one of | | sequence (e.g. | | (e.g., any one of | | | |
| | SEQ ID NOs: | | sequence fully or | | SEQ ID NOs: | | | |
| | 728-733) | | substantially | | 1-100) | | | |
| | | | complementary to | | | | | |
| | | | the guide sequence) | | | | | |
| A | H1 (e.g., | B | Passenger | C | Guide sequence | D | E | F |
| | SEQ ID NO: | | sequence (e.g. | | (e.g., any one of | | | |
| | 734) | | sequence fully or | | SEQ ID NOs: | | | |
| | | | substantially | | 1-100) | | | |
| | | | complementary to | | | | | |
| | | | the guide sequence) | | | | | |
| A | 7SK (e.g., | B | Passenger | C | Guide sequence | D | E | F |
| | SEQ ID NO: | | sequence (e.g. | | (e.g., any one of | | | |
| | 735) | | sequence fully or | | SEQ ID NOs: | | | |
| | | | substantially | | 1-100) | | | |
| | | | complementary to | | | | | |
| | | | the guide sequence) | | | | | |

Table 9 Key:
A = AAV 5′ ITR sequence selected from SEQ ID NOs: 746 and 747;
B = stem-loop 5′ flanking sequence selected from the group consisting of SEQ ID NOs: 752, 754, 756, 759, 762, 765, and 768;
C = microRNA loop sequence selected from the group consisting of SEQ ID NOs: 758, 761, 764, 767, and 770;
D = stem-loop 3′ flanking sequence selected from the group consisting of SEQ ID NOs: 753, 754, 757, 760, 763, 766, and 769;
E = 3′ untranslated region (UTR) containing a polynucleotide selected from SEQ ID NOs: 750 and 751;
F = AAV 3′ ITR sequence selected from SEQ ID NOs: 748 and 749.

In another example, the passenger sequence that is substantially complementary to the ASO sequence of any one of SEQ ID NOs: 1-100 has no more than (e.g., no more than 5, 4, 3, 2, or 1) mismatched nucleotides (i.e., mismatches) relative to the ASO sequence of any one of SEQ ID NOs: 1-100. In another example, the passenger sequence that is substantially complementary to the ASO sequence of any one of SEQ ID NOs: 1-100 has no more than 4 (e.g., no more than 4, 3, 2, or 1) mismatches relative to the ASO sequence of any one of SEQ ID NOs: 1-100. In another example, the passenger sequence that is substantially complementary to the ASO sequence of any one of SEQ ID NOs: 1-100 has no more than 3 (e.g., no more than 3, 2, or 1) mismatches relative to the ASO sequence of any one of SEQ ID NOs: 1-100. In another example, the passenger sequence that is substantially complementary to the ASO sequence of any one of SEQ ID NOs: 1-100 has no more than 2 (e.g., no more than 2 or 1) mismatches relative to the ASO sequence of any one of SEQ ID NOs: 1-100. In yet another example, the passenger sequence that is substantially complementary to the ASO sequence of any one of SEQ ID NOs: 1-100 has no more than 1 mismatch relative to the ASO sequence of any one of SEQ ID NOs: 1-100.

In another example, the passenger sequence that is substantially complementary to the ASO sequence of any one of SEQ ID NOs: 1-100 has no more than (e.g., no more than 10, 9, 8, 7, or 6) mismatched nucleotides (i.e., mismatches) relative to the ASO sequence of any one of SEQ ID NOs: 1-100. In another example, the passenger sequence that is substantially complementary to the ASO sequence of any one of SEQ ID NOs: 1-100 has no more than 9 (e.g., no more than 9, 8, 7, or 6) mismatches relative to the ASO sequence of any one of SEQ ID NOs: 1-100. In another example, the passenger sequence that is substantially complementary to the ASO sequence of any one of SEQ ID NOs: 1-100 has no more than 8 (e.g., no more than 8, 7, or 6) mismatches relative to the ASO sequence of any one of SEQ ID NOs: 1-100. In another example, the passenger sequence that is substantially complementary to the ASO sequence of any one of SEQ ID NOs: 1-100 has no more than 7 (e.g., no more than 7 or 6) mismatches relative to the ASO sequence of any one of SEQ ID NOs: 1-100. In another example, the passenger sequence that is substantially complementary to the ASO sequence of any one of SEQ ID NOs: 1-100 has no more than 6 mismatches relative to the ASO sequence of any one of SEQ ID NOs: 1-100. In another example, the passenger sequence that is substantially complementary to the ASO sequence of any one of SEQ ID NOs: 1-100 has no more than mismatches relative to the ASO sequence of any one of SEQ ID NOs: 1-100. In another example, the passenger sequence that is substantially complementary to the ASO sequence of any one of SEQ ID NOs: 1-100 has no more than 4 mismatches relative to the ASO sequence of any one of SEQ ID NOs: 1-100. In another example, the passenger sequence that is substantially complementary to the ASO sequence of any one of SEQ ID NOs: 1-100 has no more than 3 mismatches relative to the ASO sequence of any one of SEQ ID NOs: 1-100. In another example, the passenger sequence that is substantially complementary to the ASO sequence of any one of SEQ ID NOs: 1-100 has no more than 2 mismatches relative to the ASO sequence of any one of SEQ ID NOs: 1-100. In another example, the passenger sequence that is substantially complementary to the ASO sequence of any one of SEQ ID NOs: 1-100 has no more than 1 mismatch relative to the ASO sequence of any one of SEQ ID NOs: 1-100.

The expression constructs exemplified in Table 9 or Table of U.S. Provisional Patent Application No.: 63/050,742, which is incorporated herein by reference in its entirety, may further include additional vector elements such as, e.g., regulatory sequences (e.g., one or more enhancer sequence, terminator sequence, or a WPRE sequence), stuffer and linker sequences between or within any of the described elements, as well as any other conventional expression construct element known in the art that can be used to promote the expression of a heterologous polynucleotide in a cell. Table 9 and Table provide exemplary expression cassette, each of which are shown within a single row and designated by an identifier number (e.g., exemplary expression cassette configurations 1-3800 of Table 9 and configurations 1-2000 of Table 10), and each element of the expression cassette represented in a series of columns oriented in the 5′ to 3′ direction.

An exemplary monocistronic (i.e., single ASO-encoding) construct of the disclosure may include an AAV (e.g., AAV9) construct containing an hSyn promoter (SEQ ID NO: 790) and ASO G9 (SEQ ID NO: 68) incorporated into an A-miR-scaffold (Construct 1; see FIG. 6A). Such a construct may have the nucleic acid sequence of SEQ ID NO: 775 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 775 (see below).

(SEQ ID NO: 775)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACCAGGGTAATGGGGATCCTCTAGATCCGGTCGGG
CCCGCGGTACCGTCGAGAAGCTTGATGTGGGCGGAGCTTCGAAGGGGCGG
GCGCCCGTGGGGCGGGTCCTGAGTGGGGGCGGGACCGGGGCCGGCACCTG
GGTGAGGTTCTGCAGAGGGCCCTGCGTATGAGTGCAAGTGGGTTTTAGGA
CCAGGATGAGGCGGGGTGGGGGTGCCTACCTGACGACCGACCCCGACCCA
CTGGACAAGCACCCAACCCCCATTCCCCAAATTGCGCATCCCCTATCAGA
GAGGGGGAGGGGAAACAGGATGCGGCGAGGCGCGTGCGCACTGCCAGCTT
CAGCACCGCGGACAGTGCCTTCGCCCCCGCCTGGCGGCGCGCGCCACCGC
CGCCTCAGCACTGAAGGCGCGCTGACGTCACTCGCCGGTCCCCCGCAAAC
TCCCCTTCCCGGCCACCTTGGTCGCGTCCGCGCCGCCGCCGGCCCAGCCG
GACCGCACCACGCGAGGCGCGAGATAGGGGGGCACGGGCGCGACCATCTG
CGCTGCGGCGCCGGCGACTCAGCGCTGCCTCAGTCTGCCAATTGCAGCGG
AGGAGTCGTGTCGTGCCTGAGAGCGCAGGGCGCGCCTAGCCCGGGCTAGG
TCGACTCGACTAGGGATAACAGGGTAATTGTTTGAATGAGGCTTCAGTAC
TTTACAGAATCGTTGCCTGCACATCTTGGAAACACTTGCTGGGATTACTT
CTTCAGGTTAACCCAACAGAAGGCTCGAGAAGGTATATTGCTGTTGACAG
TGAGCGCT [symbol] [symbol] TAGTGAAGCCACAGATG [symbol]
TTGCCTACTGCCTCGGAATTCAAGGG
GCTACTTTAGGAGCAATTATCTTGTTTACTAAAACTGAATACCTTGCTAT
CTCTTTGATACATTTTTACAAAGCTGAATTAAAATGGTATAAATTATCAC
GGGATCCGATCTTTTTCCCTCTGCCAAAAATTATGGGGACATCATGAAGC
CCCTTGAGCATCTGACTTCTGGCTAATAAAGGAAATTTATTTTCATTGCA
ATAGTGTGTTGGAATTTTTTGTGTCTCTCACTCGGCGGCCGCCCGAGTTT
AATTGGTTTATAGAACTCTTCAAGCTAGCGAAGCAATTCGTTGATCTGAA
TTTCGACCACCCATAATACCCATTACCCTGGTAGATAAGTAGCATGGCGG
GTTAATCATTAACTACAaggaacccctagtgatggagttggccactccct
ctctgcgcgctcgctcgctcactgaggccgggcgaccaaaggtcgcccga
cgcccgggctttgcccgggcggcctcagtgagcgagcgagcgcgcag
Key: Bold = 5′ ITR sequence; single underline =
promoter sequence; italics = 5′ flanking sequence-
guide sequence-microRNA loop sequence-passenger
sequence-3′ flanking sequence; italics + bold +
single underline: DNA encoding the G9 guide
sequence; italics + wave underline: DNA encoding
the G9 passenger sequence; double underline:
polyA sequence; bold + lowercase letters: 3′ ITR
sequence(SEQ ID NO: 789).

The exemplary monocistronic, anti-Grik2 construct described above may include the Grik2 antisense guide sequence (e.g., G9, SEQ ID NO: 68) incorporated into an A-miR-scaffold, such that the microRNA coding sequence is a polynucleotide having the nucleic acid sequence of SEQ ID NO: 795 or is a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 795.

(SEQ ID NO: 795)
GTTTGAATGAGGCTTCAGTACTTTACAGAA
TCGTTGCCTGCACATCTTGG
AAACACTTGCTGGGATTACTTCTTCAGGTTA
ACCCAACAGAAGGCTCGAG
AAGGTATATTGCTGTTGACAGTGAGCGC [symbol] [symbol]
TAGTGAAGCCACAGATG [symbol]
TTGCCTACTG
CCTCGGAATTCAAGGGGCTACTTTAGGAGCA
ATTATCTTGTTTACTAAAA
CTGAATACCTTGCTATCTCTTTGATACATTTTTACAAAGC
TGAATTAAAATGGTATAAATTA
Key: italics = 5′ flanking sequence-guide
sequence-microRNA loop sequence-passenger
sequence-3′ flanking sequence; italics + wave
underline: DNA encoding the passenger sequence;
italics + bold + single underline: DNA encoding
the G9 guide sequence.

Another exemplary monocistronic, anti-Grik2 construct of the disclosure may include AAV (e.g., AAV9) constructs containing a C1ql2 promoter (SEQ ID NO: 791) and an hSyn promoter (SEQ ID NO: 790) in tandem and ASO G9 (SEQ ID NO: 68) incorporated into an A-miR-scaffold (Construct 2; see FIG. 6B). Such a construct may have the nucleic acid sequence of SEQ ID NO: 777 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 777 (see below).

(SEQ ID NO: 777)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACCAGGGTAATGGGGATCCTCTAGATCCGGTCGGG
CCCGCGGTACCGTCGAGAAGCTTGATGTGGGCGGAGCTTCGAAGGGGCGG
GCGCCCGTGGGGCGGGTCCTGAGTGGGGGCGGGACCGGGGCCGGCACCTG
GGTGAGGTTCGATCCTATCACGAGACTAGCCTCGAGAAGCTTGATATCAG
CACCCACATAGCAGCTCACAAATGTCTGAAACTCCAATTCTTGGGAATCT
GACACGATCACACATGCAGGCAAAATACCAATGTACATGAATTAAAAAAA
AAAAAAACAACCTTTAAAAGAAACAAGGGTTCAGTACCACTACTGACATC
TTGTTTCCCCAGAGGCCTTACTTTAATTATTTATTGTTTCCACTTAGTTG
CTCAATTAATTAATTTAGAGGTTTTTTTCTTCCTTTCTTTTTCTTTTTTC
TTTCTCTCTTTTTTTTCTTCTTAAGACAGGGTTTCTCTGTGTAGCTCAGG
CTATCCTGGAACTCACTCTGTAGACCAGGCTGGCCTTGTACTCAAAGATC
TGCCTGCCTCTGCCTCCCCAGTGCTGGGATTAAAGACATGCACCATCACT
GCCCTGCTTTCCTCTTTTTATTTTGAAAATTGTTCATCAACAGTTACTAA
ACGTGTTCGAATTCCAAGAGCTGACTAGACATATAAGACCATTCAGCCTT
CTGAATAAGATGTAGGTGTGCCCCTCCTCTTACTCCTCTATTTGGAAGTT
GGTTACTTTCTGTATGTAGTATGCGAATCCCCCTCTGCCACCCCGCTTTC
TGTTTTAAAACAGAAAAGGCTGCAACATACAGTGTGTGCTTCTGTTCTTG
AACTGGAAGCTTAGGCTGTCCTGGACTTGGGTTGAGACCTGGGCTCATCC
AGATAGGAAATGGATTTGGTGACCCCGCCAGGACTTCGCAGGCACCACAT
CGTGGTCGTGTGTGGGTGCTGTATGCACCCACTGATTGCGCGCGTGGGTT
CCAGAGCTTGGTGGTCTGCGAGAGGAGAGTGGGCAAGAGTGGGTGTGTCT
GTGGAGCCCCAGCTAGGGGCTGCTGCCCGCTGCTCCCACTTGTGGCTCCT
GGGCGCCGCCAGCAGGCACATCTCCGGAGGACGCCGCGGGATGGGAGCTG
ATGACAGGAGAGCGCCGTCTCCCGAGTGATGGCAGCGCACGCTGCTGCCT
CGCCGCCTCCGCCGCTCAGTCCTGATCTTACGTTAGGGTAGCTGGGTACC
CCCTCCGCCCGGGAACCAGCTAGTAGAGGGAGAACAGAGCAGAGCGTGCG
GCAGAGCCGATCCCGCGTCCCGCCGAACCCTGCCAAGCCCCGCCAATCCC
AGCAGAGCAGGAACCAGCGCAGCTGAGCCAACACCGGACGCCGCACTGAG
ACCCAGCATTCCCCAGCCGCCACTACCCGGTCCCCGCCGGGGTGCCGGGC
TCGTCCTGTGAGCCCCTCGTCATGCGTGTCGGGCTCTTCGACTCTCCAGA
TCAGTTCCAGAGCGCTGAGGGCCCTGCGTATGAGTGCAAGTGGGTTTTAG
GACCAGGATGAGGCGGGGTGGGGGTGCCTACCTGACGACCGACCCCGACC
CACTGGACAAGCACCCAACCCCCATTCCCCAAATTGCGCATCCCCTATCA
GAGAGGGGGAGGGGAAACAGGATGCGGCGAGGCGCGTGCGCACTGCCAGC
TTCAGCACCGCGGACAGTGCCTTCGCCCCCGCCTGGCGGCGCGCGCCACC
GCCGCCTCAGCACTGAAGGCGCGCTGACGTCACTCGCCGGTCCCCCGCAA
ACTCCCCTTCCCGGCCACCTTGGTCGCGTCCGCGCCGCCGCCGGCCCAGC
CGGACCGCACCACGCGAGGCGCGAGATAGGGGGGCACGGGCGCGACCATC
TGCGCTGCGGCGCCGGCGACTCAGCGCTGCCTCAGTCTGCCAATTGCAGC
GGAGGAGTCGTGTCGTGCCTGAGAGCGCAGGGCGCGCCTAGCCCGGGCTA
GGTCGACTCGACTAGGGATAACAGGGTAATTGTTTGAATGAGGCTTCAGT
ACTTTACAGAATCGTTGCCTGCACATCTTGGAAACACTTGCTGGGATTAC
TTCTTCAGGTTAACCCAACAGAAGGCTCGAGAAGGTATATTGCTGTTGAC
AGTGAGCGCT [symbol] [symbol] TAGTGAAGCCACAGATG [symbol]
ATTGCCTACTGCCTCGGAATTCAAGGG
GCTACTTTAGGAGCAATTATCTTGTTTACTAAAACTGAATACCTTGCTAT
CTCTTTGATACATTTTTACAAAGCTGAATTAAAATGGTATAAATTATCAC
GGGATCCGATGTTTTTCCCTCTGCCAAAAATTATGGGGACATCATGAAGC
CCCTTGAGCATCTGACTTCTGGCTAATAAAGGAAATTTATTTTCATTGCA
ATAGTGTGTTGGAATTTTTTGTGTCTCTCACTCGGCGGCCGCCCGAGTTT
AATTGGTTTATAGAACTCTTCAAGCTAGCGAAGCAATTCGTTGATCTGAA
TTTCGACCACCCATAATACCCATTACCCTGGTAGATAAGTAGCATGGCGG
GTTAATCATTAACTACAaggaacccctagtgatggagttggccactccct
ctctgcgcgctcgctcgctcactgaggccgggcgaccaaaggtcgcccga
cgcccgggctttgcccgggcggcctcagtgagcgagcgagcgcgcag
Key: Bold = 5′ ITR sequence; single underline +
italics = C1ql2 promoter sequence; single
underline: hSyn promoter sequence; italics = 5′
flanking sequence-guide sequence-microRNA loop
sequence-passenger sequence-3′ flanking sequence;
italics + wave underline: DNA encoding the
passenger sequence; italics + bold + single
underline: DNA encoding the G9 guide sequence;
double underline: polyA sequence; bold + lowercase
letters: 3′ ITR sequence (SEQ ID NO: 789).

The exemplary monocistronic, anti-Grik2 construct described above may include the Grik2 antisense guide sequence (e.g., G9, SEQ ID NO: 68) incorporated into a microRNA scaffold, such that the microRNA coding sequence is a polynucleotide having the nucleic acid sequence of SEQ ID NO: 778 20 or is a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 778.

(SEQ ID NO: 778)
GTTTGAATGAGGCTTCAGTACTTTACAGAATCGTTGCCTGCACATCTTGG
AAACACTTGCTGGGATTACTTCTTCAGGTTAACCCAACAGAAGGCTCGAG
AAGGTATATTGCTGTTGACAGTGAGCGCT [symbol] [symbol]
TAGTGAAGCCACAGATG [symbol] ATTGCCTACTG
CCTCGGAATTCAAGGGGCTACTTTAGGAGCAATTATCTTGTTTACTAAAA
CTGAATACCTTGCTATCTCTTTGATACATTTTTACAAAGCTGAATTAAAA
TGGTATAAATTA
Key: italics = 5′ flanking sequence-guide
sequence-microRNA loop sequence-passenger
sequence-3′ flanking sequence; italics + wave
underline: DNA encoding the passenger sequence;
italics + bold + single underline: DNA encoding
the G9 guide sequence.

Another exemplary monocistronic, anti-Grik2 construct of the disclosure may include self-complementary (sc)AAV (e.g., scAAV9) constructs containing an hSyn promoter (SEQ ID NO: 790) downstream of the 5′ ITR sequence and ASO G9 (SEQ ID NO: 68) incorporated into an A-miR-scaffold (Construct 3; see FIG. 6C). Such a construct may have the nucleic acid sequence of SEQ ID NO: 779 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 779 (see below).

(SEQ ID NO: 779)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGAATTCACGCGTGGTACCCTGCAGAGGGCCCTGCGTATGAGTGCA
AGTGGGTTTTAGGACCAGGATGAGGCGGGGTGGGGGTGCCTACCTGACGA
CCGACCCCGACCCACTGGACAAGCACCCAACCCCCATTCCCCAAATTGCG
CATCCCCTATCAGAGAGGGGGAGGGGAAACAGGATGCGGCGAGGCGCGTG
CGCACTGCCAGCTTCAGCACCGCGGACAGTGCCTTCGCCCCCGCCTGGCG
GCGCGCGCCACCGCCGCCTCAGCACTGAAGGCGCGCTGACGTCACTCGCC
GGTCCCCCGCAAACTCCCCTTCCCGGCCACCTTGGTCGCGTCCGCGCCGC
CGCCGGCCCAGCCGGACCGCACCACGCGAGGCGCGAGATAGGGGGGCACG
GGCGCGACCATCTGCGCTGCGGCGCCGGCGACTCAGCGCTGCCTCAGTCT
GCCAATTGCAGCGGAGGAGTCGTGTCGTGCCTGAGAGCGCAGGGCGCGCC
TAGCCCGGGCTAGGTCGACTCGACTAGGGATAACAGGGTAATTGTTTGAA
TGAGGCTTCAGTACTTTACAGAATCGTTGCCTGCACATCTTGGAAACACT
TGCTGGGATTACTTCTTCAGGTTAACCCAACAGAAGGCTCGAGAAGGTAT
ATTGCTGTTGACAGTGAGCGCT [symbol] [symbol] TAGTGA
AGCCACAGATG [symbol] TTGCCTACTGCCTCGG
AATTCAAGGGGCTACTTTAGGAGCAATTATCTTGTTTACTAAAACTGAAT
ACCTTGCTATCTCTTTGATACATTTTTACAAAGCTGAATTAAAATGGTAT
AAATTATCACGGGATCCAAGCTTGATCTTTTTCCCTCTGCCAAAAATTAT
GGGGACATCATGAAGCCCCTTGAGCATCTGACTTCTGGCTAATAAAGGAA
ATTTATTTTCATTGCAATAGTGTGTTGGAATTTTTTGTGTCTCTCACTCG
GCTAGCGAAGCAATTC [symbol] [symbol] AGATCGATCTGAGga
acccctagtgatggagttggccactccctctctgcgcgctcgctcgctca
ctgaggccgcccgggcaaagcccgggcgtcgggcgacctttggtcgcccg
gcctcagtgagcgagcgagcgcgcagagagggagtggccaa
Key: Bold = 5′ ITR sequence; single underline +
italics = hSyn promoter sequence; single
underline: hSyn promoter sequence; italics = 5′
flanking sequence-guide sequence-microRNA loop
sequence-passenger sequence-3′ flanking sequence;
italics + wave underline: DNA encoding the
passenger sequence; italics + bold + single
underline: DNA encoding the G9 guide sequence;
double underline: RBG polyA sequence; double
underline + bold = BGH polyA sequence (SEQ ID NO:
793); bold + lowercase letters: 3′ ITR sequence
(SEQ ID NO: 748).

The exemplary monocistronic, anti-Grik2 construct described above may include the Grik2 antisense guide sequence (e.g., G9, SEQ ID NO: 68) incorporated into a microRNA scaffold, such that the microRNA coding sequence is a polynucleotide having the nucleic acid sequence of SEQ ID NO: 780 or is a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 780.

(SEQ ID NO: 780)
GTTTGAATGAGGCTTCAGTACTTTACAGAATCGTTGCCTGCACATCTTGGAAACACTTGCTGGGATTA
CTTCTTCAGGTTAACCCAACAGAAGGCTCGAGAAGGTATATTGCTGTTGACAGTGAGCGCT [symbol] [symbol]
TAGTGAAGCCACAGATG [symbol] ATTGCCTACTGCCT
CGGAATTCAAGGGGCTACTTTAGGAGCAATTATCTTGTTTACTAAAACTGAATACCTTGCTATCTCTTT
GATACATTTTTACAAAGCTGAATTAAAATGGTATAAATTA

Key:
italics = 5’ flanking sequence-guide sequence-microRNA loop sequence-passenger sequence-3’ flanking sequence; italics + wave underline: DNA encoding the passenger sequence; italics + bold + single underline: DNA encoding the G9 guide sequence.

Another exemplary monocistronic, anti-Grik2 construct of the disclosure may include an scAAV (e.g., scAAV9) construct containing an hSyn promoter (SEQ ID NO: 790) proximal to the 3′ ITR (“FLIP”) and ASO G9 (SEQ ID NO: 68) incorporated into an A-miR-scaffold (Construct 4; see FIG. 6D). Such a construct may have the nucleic acid sequence of SEQ ID NO: 781 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 781 (see below).

(SEQ ID NO: 781)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGAATTCACGCGTGGTACCCGGCC
GCCGAGTGAGAGACACAAAAAATTCCAACACACTATTGCAATGAAAATAAATTTCCTTTATTAGCCAG
AAGTCAGATGCTCAAGGGGCTTCATGATGTCCCCATAATTTTTGGCAGAGGGAAAAAGATCGGATCC
CGTGATAATTTATACCATTTTTAATTCAGCTTTGTAAAAATGTATCAAAGAGATAGCAAGGTATTCAGTT
TTAGTAAACAAGATAATTGCTCCTAAAGTAGCCCCTTGAATTCCGAGGCAGTAGGCAAT [symbol] [symbol]
CATCTGTGGCTTCACTA [symbol] AGCGCTCACTGTCAAC
AGCAATATACCTTCTCGAGCCTTCTGTTGGGTTAACCTGAAGAAGTAATCCCAGCAAGTGTTTCCAAG
ATGTGCAGGCAACGATTCTGTAAAGTACTGAAGCCTCATTCAAACAATTACCCTGTTATCCCTAGTCG
AGTCGACCTAGCCCGGGCTAGGCGCGCCCTGCGCTCTCAGGCACGACACGACTCCTCCGCTGCAA
TTGGCAGACTGAGGCAGCGCTGAGTCGCCGGCGCCGCAGCGCAGATGGTCGCGCCCGTGCCCCC
CTATCTCGCGCCTCGCGTGGTGCGGTCCGGCTGGGCCGGCGGCGGCGCGGACGCGACCAAGGTG
GCCGGGAAGGGGAGTTTGCGGGGGACCGGCGAGTGACGTCAGCGCGCCTTCAGTGCTGAGGCGG
CGGTGGCGCGCGCCGCCAGGCGGGGGCGAAGGCACTGTCCGCGGTGCTGAAGCTGGCAGTGCGC
ACGCGCCTCGCCGCATCCTGTTTCCCCTCCCCCTCTCTGATAGGGGATGCGCAATTTGGGGAATGG
GGGTTGGGTGCTTGTCCAGTGGGTCGGGGTCGGTCGTCAGGTAGGCACCCCCACCCCGCCTCATC
CTGGTCCTAAAACCCACTTGCACTCATACGCAGGGCCCTCTGCAGGCTAGCGAAGCAATTC [symbol] [symbol]
AGATCGATCTGAGgaacccctagtgatggagttggccactccctctctgcgcgctcgctcgctca
ctgaggccgcccgggcaaagcccgggcgtcgggcgacctttggtcgcccggcctcagtgagcgagcgagcgcgcagagaggga
gtggccaa

Key:
Bold = 5’ ITR sequence; single underline + italics = hSyn promoter sequence; single underline: hSyn promoter sequence; italics = 5’ flanking sequence-guide sequence-microRNA loop sequence-passenger sequence-3’ flanking sequence; italics + wave underline: DNA encoding the passenger sequence; italics + bold + single underline: DNA encoding the G9 guide sequence; double underline: RBG polyA sequence; double underline + bold = BGH polyA sequence (SEQ ID NO: 793); bold + lowercase letters: 3’ ITR sequence (SEQ ID NO: 748). Note that the guide sequence in this case is the “reverse” as the entire cassette is reads 3’ ITR to 5’ITR instead of 5’ ITR to 3’ ITR.

The exemplary monocistronic, anti-Grik2 construct described above may include the Grik2 antisense guide sequence (e.g., G9, SEQ ID NO: 68) incorporated into a microRNA scaffold, such that the microRNA coding sequence is a polynucleotide having the nucleic acid sequence of SEQ ID NO: 782 (top strand) or SEQ ID NO: 794 (bottom strand) or is a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 782 or SEQ ID NO: 794.

(SEQ ID NO: 782)
TAATTTATACCATTTTAATTCAGCTTTGTAAAAATGTATCAAAGAGATAGCAAGGTATTCAGTTTTAGTA
AACAAGATAATTGCTCCTAAAGTAGCCCCTTGAATTCCGAGGCAGTAGGCAAT [symbol] [symbol]
CATCTGTGGCTTCACTA [symbol] AGCGCTCACTGTCAACAGCAAT
ATACCTTCTCGAGCCTTCTGTTGGGTTAACCTGAAGAAGTAATCCCAGCAAGTGTTTCCAAGATGTG
CAGGCAACGATTCTGTAAAGTACTGAAGCCTCATTCAAAC
(SEQ ID NO: 794)
TCGACTAGGGATAACAGGGTAATTGTTTGAATGAGGCTTCAGTACTTTACAGAATCGTTGCCTGCAC
ATCTTGGAAACACTTGCTGGGATTACTTCTTCAGGTTAACCCAACAGAAGGCTCGAGAAGGTATATT
GCTGTTGACAGTGAGCGCT [symbol] TAGTGAAGCCACAGATG [symbol] [symbol]
ATTGCCTACTGCCTCGGAATTCAAGGGGCTACTTTAGGAGCAATTATCTTGTTTAC
TAAAACTGAATACCTTGCTATCTCTTTGATACATTTTTACAAAGCTGAATTAAAATGGTATAAATTATCA
C

Key:
italics = 5’ flanking sequence-guide sequence-microRNA loop sequence-passenger sequence-3’ flanking sequence; italics + wave underline: DNA encoding the passenger sequence; italics + bold + single underline: DNA encoding the G9 guide sequence.

Another exemplary monocistronic, anti- Grik2 construct of the disclosure may include an AAV (e.g., AAV9) construct containing an hSyn promoter (SEQ ID NO: 790) and three concatenated copies of ASO G9 (SEQ ID NO: 68) incorporated into an A-miR-scaffold (Construct 5; see FIG. 6E). Such a construct may have the nucleic acid sequence of SEQ ID NO: 783 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 783 (see below).

(SEQ ID NO: 783)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGATCCGGT
CGGGCCCGCGGTACCGTCGAGAAGCTTGATGTGGGCGGAGCTTCGAAGGGGCGGGCGCCCGTGG
GGCGGGTCCTGAGTGGGGGCGGGACCGGGGCCGGCACCTGGGTGAGGTTCTGCAGAGGGCCCTG
CGTATGAGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCAGAGAGGG
GGAGGGGAAACAGGATGCGGCGAGGCGCGTGCGCACTGCCAGCTTCAGCACCGCGGACAGTGCC
TTCGCCCCCGCCTGGCGGCGCGCGCCACCGCCGCCTCAGCACTGAAGGCGCGCTGACGTCACTC
GCCGGTCCCCCGCAAACTCCCCTTCCCGGCCACCTTGGTCGCGTCCGCGCCGCCGCCGGCCCAGC
CGGACCGCACCACGCGAGGCGCGAGATAGGGGGGCACGGGCGCGACCATCTGCGCTGCGGCGCC
GGCGACTCAGCGCTGCCTCAGTCTGCCAATTGCAGCGGAGGAGTCGTGTCGTGCCTGAGAGCGCA
GGGCGCGCCTAGCCCGGGCTAGGTCGAC^{∧}TCGACTAGGGATAACAGGGTAATTGTTTGAATGAGGC
TTCAGTACTTTACAGAATCGTTGCCTGCACATCTTGGAAACACTTGCTGGGATTACTTCTTCAGGTTA
ACCCAACAGAAGGCTCGAGAAGGTATATTGCTGTTGACAGTGAGCGCT [symbol] [symbol]
TAGTGAAGCCACAGATG [symbol] TTGCCTACTGCCTCGGAATTCAAGGG
GCTACTTTAGGAGCAATTATCTTGTTTACTAAAACTGAATACCTTGCTATCTCTTTCATACATTTTTACA
AAGCTGAATTAAAATGGTATAAATTATCACGGGATCC^{∧}GGTCGAC*TCGACTAGGGATAACAGGGTAA
TTGTTTGAATGAGGCTTCAGTACTTTACAGAATCGTTGCCTGCACATCTTGGAAACACTTGCTGGGAT
TACTTCTTCAGGTTAACCCAACAGAAGGCTCGAGAAGGTATATTGCTGTTGACAGTGAGCGCT [symbol] [symbol]
TAGTGAAGCCACAGATG [symbol] TTGCCTACTGC
CTCGGAATTCAAGGGGCTACTTTAGGAGCAATTATCTTGTTTACTAAAACTGAATACCTTGCTATCTC
TTTGATACATTTTTACAAAGCTGAATTAAAATGGTATAAATTATCACGGGATCC*GGTCGAC#TCGACT
AGGGATAACAGGGTAATTGTTTGAATGAGGCTTCAGTACTTTACAGAATCGTTGCCTGCACATCTTG
GAAACACTTGCTGGGATTACTTCTTCAGGTTAACCCAACAGAAGGCTCGAGAAGGTATATTGCTGTT
GACAGTGAGCGCT [symbol] TAGTGAAGCCACAGATG [symbol] [symbol]
TTGCCTACTGCCTCGGAATTCAAGGGGCTACTTTAGGAGCAATTATCTTGTTTACTAAAAC
TGAATACCTTGCTATCTCTTTGATACATTTTTACAAAGCTGAATTAAAATGGTATAAATTATCACGGGA
TCC#GATCTTTTTCCCTCTGCCAAAAATTATGGGGACATCATGAAGCCCCTTGAGCATCTGACTTCTG
GCTAATAAAGGAAATTTATTTTCATTGCAATAGTGTGTTGGAATTTTTTGTGTCTCTCACTCGGCGGC
CGCCCGAGTTTAATTGGTTTATAGAACTCTTCAAGCTAGCgaagcaattcgttgatctgaatttcgaccacccataat
acccattaccctggtagataagtagcatggcgggttaatcattaactacaaggaacccctagtgatggagttggccactccctctctgcg
cgctcgctcgctcactgaggccgggcgaccaaaggtcgcccgacgcccgggctttgcccgggcggcctcagtgagcgagcgagc
gcgcag

Key:
Bold = 5’ ITR sequence; single underline = promoter sequence; italics = 5’ flanking sequence-guide sequence-microRNA loop sequence-passenger sequence-3’ flanking sequence; italics + wave underline: DNA encoding the passenger sequence; italics + bold + single underline: DNA encoding the G9 guide sequence; double underline: polyA sequence; bold + lowercase letters: 3’ ITR sequence (SEQ ID NO: 789); ^{∧} = boundaries of the first concatemer; * = boundaries of the second concatemer; and # = boundaries of the third concatemer.

Another exemplary monocistronic, anti-Grik2 construct of the disclosure may include an AAV (e.g., AAV9) construct containing an hSyn promoter (SEQ ID NO: 790) and three concatenated copies different antisense sequences, including G9 (SEQ ID NO: 68), GI (SEQ ID NO: 77), MU (SEQ ID NO: 96), each incorporated into an A-miR-scaffold (Construct 6; see FIG. 6E). Such a construct may have the nucleic acid sequence of SEQ ID NO: 784 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 784 (see below).

(SEQ ID NO: 784)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGATCCGGT
CGGGCCCGCGGTACCGTCGAGAAGCTTGATGTGGGCGGAGCTTCGAAGGGGCGGGCGCCCGTGG
GGCGGGTCCTGAGTGGGGGCGGGACCGGGGCCGGCACCTGGGTGAGGTTCTGCAGAGGGCCCTG
CGTATGAGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCAGAGAGGG
GGAGGGGAAACAGGATGCGGCGAGGCGCGTGCGCACTGCCAGCTTCAGCACCGCGGACAGTGCC
TTCGCCCCCGCCTGGCGGCGCGCGCCACCGCCGCCTCAGCACTGAAGGCGCGCTGACGTCACTC
GCCGGTCCCCCGCAAACTCCCCTTCCCGGCCACCTTGGTCGCGTCCGCGCCGCCGCCGGCCCAGC
CGGACCGCACCACGCGAGGCGCGAGATAGGGGGGCACGGGCGCGACCATCTGCGCTGCGGCGCC
GGCGACTCAGCGCTGCCTCAGTCTGCCAATTGCAGCGGAGGAGTCGTGTCGTGCCTGAGAGCGCA
GGGCGCGCCTAGCCCGGGCTAGGTCGAC^{∧}TCGACTAGGGATAACAGGGTAATTGTTTGAATGAGGC
TTCAGTACTTTACAGAATCGTTGCCTGCACATCTTGGAAACACTTGCTGGGATTACTTCTTCAGGTTA
ACCCAACAGAAGGCTCGAGAAGGTATATTGCTGTTGACAGTGAGCGCT [symbol] [symbol]
TAGTGAAGCCACAGATG [symbol] ATTGCCTACTGCCTCGGAATTCAAGGG
GCTACTTTAGGAGCAATTATCTTGTTTACTAAAACTGAATACCTTGCTATCTCTTTGATACATTTTTACA
AAGCTGAATTAAAATGGTATAAATTATCACGGGATCC^{∧}GGTCGAC*TCGACTAGGGATAACAGGGTAA
TTGTTTGAATGAGGCTTCAGTACTTTACAGAATCGTTGCCTGCACATCTTGGAAACACTTGCTGGGAT
TACTTCTTCAGGTTAACCCAACAGAAGGCTCGAGAAGGTATATTGCTGTTGACAGTGAGCGAC [symbol] [symbol]
TAGTGAAGCCACAGATG [symbol] GCTGCCTACTG
CCTCGGAATTCAAGGGGCTACTTTAGGAGCAATTATCTTGTTTACTAAAACTGAATACCTTGCTATCT
CTTTGATACATTTTTACAAAGCTGAATTAAAATGGTATAAATTATCACGGGATCC*GGTCGAC#TCGAC
TAGGGATAACAGGGTAATTGTTTGAATGAGGCTTCAGTACTTTACAGAATCGTTGCCTGCACATCTTG
GAAACACTTGCTGGGATTACTTCTTCAGGTTAACCCAACAGAAGGCTCGAGAAGGTATATTGCTGTT
GACAGTGAGCGAA [symbol] TAGTGAAGCCACAGATG [symbol] [symbol]
TCTGCCTACTGCCTCGGAATTCAAGGGGCTACTTTAGGAGCAATTATCTTGTTTACTAAAACT
GAATACCTTGCTATCTCTTTGATACATTTTTACAAAGCTGAATTAAAATGGTATAAATTATCACGGGAT
CC#GATCTTTTTCCCTCTGCCAAAAATTATGGGGACATCATGAAGCCCCTTGAGCATCTGACTTCTGG
CTAATAAAGGAAATTTATTTTCATTGCAATAGTGTGTTGGAATTTTTTGTGTCTCTCACTCGGCGGCC
GCCCGAGTTTAATTGGTTTATAGAACTCTTCAAGCTAGCgaagcaattcgttgatctgaatttcgaccacccataatac
ccattaccctggtagataagtagcatggcgggttaatcattaactacaaggaacccctagtgatggagttggccactccctctctgcgcg
ctcgctcgctcactgaggccgggcgaccaaaggtcgcccgacgcccgggctttgcccgggcggcctcagtgagcgagcgagcgc
gcag

Key: Bold = 5’ ITR sequence; single underline = promoter sequence; italics = 5’ flanking sequence-guide sequence-microRNA loop sequence-passenger sequence-3’ flanking sequence; italics + wave underline: DNA encoding the passenger sequence complementary to a guide sequence (G9, GI, MU-in that order); italics + bold + single underline: DNA encoding the guide sequence (G9, GI, MU-in that order); double underline: polyA sequence; bold + lowercase letters: 3’ ITR sequence (SEQ ID NO: 789); ^{∧} = boundaries of the first concatemer; * = boundaries of the second concatemer; and # = boundaries of the third concatemer.

In other cases, the G9 ASO sequence (SEQ ID NO: 68) may be incorporated into an E-miR-124-3 scaffold, such that the microRNA coding sequence is a polynucleotide having the nucleic acid sequence of SEQ ID NO: 801 or is a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 801.

(SEQ ID NO: 801)
TCTGCCGCGGAAAGGGGAGAAGTGTGGGCTCCTCCGAGTCGGGGGCGGACTGGGACAGCACAGT
CGGCTGAGCGCAGCGCCCCCGCCCTGCCCGCCACGCGGCGAAGACGCCTGAGCGTTCGCGCCCC
TCGGGCGAGGACCCCACGCAAGCCCGAGCCGGTCCCGACCCTGGCCCCGACGCTCGCCGCCCGC
CCCAGCCCTGAGGGCCCCTCTA [symbol] TTTAATGTCTATACAAT [symbol] [symbol]
AGAGAGGCGCCTCCGCCGCTCCTTTCTCATGGAAATGGCCCGCGAGCCCGTC
CGGCCCAGCGCCCCTCCCGCGGGAGGAAGGCGAGCCCGGCCCCCGGCGGCCATTCGCGCCGCG
GACAAATCCGGCGAACAATGCGCCCGCCCAGAGTGCGGCCCAGCTGCCGGGCCGGGGATCTGGC
CGCGGGACACAAAGGGGCCCGCACGCCTCTGGCGT

Key:
italics = 5’ flanking sequence-guide sequence-microRNA loop sequence-passenger sequence-3’ flanking sequence; italics + wave underlind: DNA encoding the G9 passenger sequence italics + bold + single underline: DNA encoding the G9 guide sequence.

Another monocistronic, anti-Grik2 construct of the disclosure is an AAV (e.g., AAV9) construct containing an hSyn promoter (SEQ ID NO: 790) an anti-Grik2 antisense sequence G9 (SEQ ID NO: 68) incorporated into an E-miR-124-3 scaffold. Such a construct may have the nucleic acid sequence of SEQ ID NO: 809 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 809 (see below). The expression construct of SEQ ID NO: 809 may be incorporated into a vector having the nucleic acid sequence of SEQ ID NO: 810 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 810.

(SEQ ID NO: 809)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTGAATTCTCTGCCGCGGAAAGGGGAGAAGTGTGGGCTC
CTCCGAGTCGGGGGCGGACTGGGACAGCACAGTCGGCTGAGCGCAGCGCCCCCGCCCTGCCCG
CCACGCGGCGAAGACGCCTGAGCGTTCGCGCCCCTCGGGCGAGGACCCCACGCAAGCCCGAGC
CGGTCCCGACCCTGGCCCCGACGCTCGCCGCCCGCCCCAGCCCTGAGGGCCCCTCTACAATGGG
CACTAGACATGGGATTTAATGTCTATACAATCCCATAGCTAATGCCTGTTTTAGAGAGGCGCCICC
GCCGCTCCTTTCTCATGGAAATGGCCCGCGAGCCCGTCCGGCCCAGCGCCCCTCCCGCGGGAGG
AAGGCGAGCCCGGCCCCCGGCGGCCATTCGCGCCGCGGACAAATCCGGCGAACAATGCGCCCG
CCCAGAGTGCGGCCCAGCTGCCGGGCCGGGGATCTGGCCGCGGGACACAAAGGGGCCCGCACG
CCTCTGGCGTCTCGAGGGGATCCGATCTTTTTCCCTCTGCCAAAAATTATGGGGACATCATGAAGCC
CCTTGAGCATCTGACTTCTGGCTAATAAAGGAAATTTATTTTCATTGCAATAGTGTGTTGGAATTTTTT
GTGTCTCTCATCGGCGGCCGCATAGTCTATCCAGGTTGAGCATCCTGCTGGTGGTTACAAGAAACT
GTTTGAAACTGTGGAGGAACTGTCCTCGCCGCTCACAGCTCATGTAACAGGCAGGATCCCCCTCTG
GCTCACCGGCAGTCTCCTTCGATGTGGGCCAGGACTCTTTCAACTTCCATCTCACCCATTTTACCAC
CTGTTTGATGGGCAAGCCCTCCTGCACAAGTTTGACTTTAAAGAAGGACATGTCACATACCACAGAA
GGTTCATCCGCACTGATGCTTACGTACGGGCAATGACTGAGAAAAGGATCGTCATAACAGAATTTGG
CACCTGTGCTTTCCCAGATCCCTGCAAGAATATATTTTCCAGGTTTTTTTCTTACTTTCGAGGAGTAG
AGGTTACTGACAATTGCCCTTGTTAATGTCTACCCAGTGGGGGAAGATTACTACGCTTGCACAGAGA
CCAACTTTATTACAAAGATTAATCCAGAGACCTTGGAGACAATTAAGCAGGTTGATCTTTGCAACTAA
GTCTCTGTCAATGGGGCCACTGCTCACCCCCACATTGAAAATGATGGAACCGTTTACAATATTGGTA
ATTGCTTTGGAAAAAATTTTTCAATTGCCTACAACATTGTAAAGATCCCACCACTGCAAGCAGACAAG
GAAGATCCAATAAGCAAGTCAGAGATCGTTGTACAATTCCCCTGCAGTGACCGATTCAAGCCATCTT
ACGTTCATAGTTTTGGTCTGACTCCCAACTATATCGTTTTTGTGGAGACACCAGTCAAAATTAACCTG
TTCAAGTTCCTTTCTTCATGGAGTCTTTGGGGAGCCAACTACATGGATTGTTTTGAGTCCAATGAAAC
CATGGGGTTTGGCTTCATATTGCTGACAAAAAAAGGAAAAAGTACCTCAATAATAAATACAGAACTTC
TCCTTTCAACCTCTTCCATCACATCAACACCTATGAAGACAATGGGTTTCTGATTGTGGATCTCTGCT
GCTGGAAAGGATTTGAGTTTGTTTATAATTACTTATATTTAGCCAATTTACGTGAGAACTGGGAAGAG
GTGAAAAAAAATGCCAGAAAGGCTCCCCAACCTGAAGTTAGGAGATATGTACTTCCTTTGAATATTGA
CAAGGCTGACACAGGCAAGAATTTAGTCAGCTCCCCAATACAACTGCCACTGCAATTCTGTGCAGTG
ACGAGACTATCTGGCTGGAGCCTGAAGTTCTCTTTTCAGGGCCTCGTCAAGCATTTGAGTTTCCTCA
AATCAATTACCAGAAGTATTGTGGGAAACCTTACACATATGCGTATGGACTTGGCTTGAATCACTTTG
TTCCAGATAGGCTCTGTAAGCTGAATGTCAAAACTAAAGAAACTTGGGTTTGGCAAGAGCCTGATTC
ATACCCATCAGAACCCATCTTTGTTTCTCACCCAGATGCCTTGGAAGAAGATGATGGTGTAGTTCTGA
GTGTGGTGGTGAGCCCAGGAGCAGGACAAAAGCCTGCTTATCTCCTGATTCTGAATGCCAAGGACT
TAAGTGAAGTTGCCCGGGCTGAAGTGGAGATTAACATCCCTGTCACCTTTCATGGACTGTTCAAAAA
ATCTTGAccggccgccCGAGTTTAATTGGTTTATAGAACTCTTCA

Key:
single underline: promoter sequence; bold: microRNA stem-loop structure containing guide and passenger sequences; double underline: polyA sequence; italics: stutter sequence 1; italics + underline: stutter sequence 2.

Another monocistronic, anti-Grik2 construct of the disclosure is an AAV (e.g., AAV9) construct containing a hSyn promoter (SEQ ID NO: 790) and ASO GI (SEQ ID NO: 77) incorporated into an A-miR-30 scaffold. Such a construct may have the nucleic acid sequence of SEQ ID NO: 796 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 817 (see below).

(SEQ ID NO: 817)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTAGCCCGGGCTAGGTCGACTCGACTAGGGATAACAGGGT
AATTGTTTGAATGAGGCTTCAGTACTTTACAGAATCGTTGCCTGCACATCTTGGAAACACTTGCTGGG
ATTACTTCTTCAGGTTAACCCAACAGAAGGCTAAAGAAGGTATATTGCTGTTGACAGTGAGCGAC [symbol] [symbol]
CTGTGAAGCCACAGATGGG [symbol] GCTGCCT
ACTGCCTCGGACTTCAAGGGGCTACTTTAGGAGCAATTATCTTGTTTACTAAAACTGAATACCTTGCT
ATCTCTTTGATACATTTTTACAAAGCTGAATTAAAATGGTATAAATTATCACGGGATCCGATCTTTTTC
CCTCTGCCAAAAATTATGGGGACATCATGAAGCCCCTTGAGCATCTGACTTCTGGCTAATAAAGGAA
ATTTATTTTCATTGCAATAGTGTGTTGGAATTTTTTGTGTCTCTCACTCG

Key:
single underline = promoter sequence; italics = 5’ flanking sequence-guide sequence-microRNA loop sequence-passenger sequence-3’ flanking sequence; italics + wave underline: DNA encoding the GI passenger sequence; italics + bold + single underline: DNA encoding the GI guide sequence double underline: polyA sequence.

The construct of SEQ ID NO: 817 can be incorporated into a vector further containing 5′ and 3′ ITR sequences, as is shown below in SEQ ID NO: 796.

(SEQ ID NO: 796)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGATCCGGT
CGGGCCCGCGGTACCGTCGAGAAGCTTGATGTGGGCGGAGCTTCGAAGGGGCGGGCGCCCGTGG
GGCGGGTCCTGAGTGGGGGCGGGACCGGGGCCGGCACCTGGGTGAGGTTCTGCAGAGGGCCCTG
CGTATGAGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCAGAGAGGG
GGAGGGGAAACAGGATGCGGCGAGGCGCGTGCGCACTGCCAGCTTCAGCACCGCGGACAGTGCC
TTCGCCCCCGCCTGGCGGCGCGCGCCACCGCCGCCTCAGCACTGAAGGCGCGCTGACGTCACTC
GCCGGTCCCCCGCAAACTCCCCTTCCCGGCCACCTTGGTCGCGTCCGCGCCGCCGCCGGCCCAGC
CGGACCGCACCACGCGAGGCGCGAGATAGGGGGGCACGGGCGCGACCATCTGCGCTGCGGCGCC
GGCGACTCAGCGCTGCCTCAGTCTGCCAATTGCAGCGGAGGAGTCGTGTCGTGCCTGAGAGCGCA
GGGCGCGCCTAGCCCGGGCTAGGTCGACTCGACTAGGGATAACAGGGTAATTGTTTGAATGAGGCT
TCAGTACTTTACAGAATCGTTGCCTGCACATCTTGGAAACACTTGCTGGGATTACTTCTTCAGGTTAA
CCCAACAGAAGGCTCGAGAAGGTATATTGCTGTTGACAGTGAGCGAC [symbol] [symbol]
TAGTGAAGCCACAGATG [symbol] CTGCCTACTGCCTCGGAATTCAAGGG
GCTACTTTAGGAGCAATTATCTTGTTTACTAAAACTGAATACCTTGCTATCTCTTTGATACATTTTTACA
AAGCTGAATTAAAATGGTATAAATTATCACGGGATCCGATCTTTTTCCCTCTGCCAAAAATTATGGGG
ACATCATGAAGCCCCTTGAGCATCTGACTTCTGGCTAATAAAGGAAATTTATTTTCATTGCAATAGTG
TGTTGGAATTTTTTGTGTCTCTCACTCGGCGGCCGCCCGAGTTTAATTGGTTTATAGAACTCTTCAAG
CTAGCGAAGCAATTCGTTGATCTGAATTTCGACCACCCATAATACCCATTACCCTGGTAGATAAGTAG
CATGGCGGGTTAATCATTAACTACAaggaacccctagtgatggagttggccactccctctctgcgcgctcgctcgctcactg
aggccgggcgaccaaaggtcgcccgacgcccgggctttgcccgggcggcctcagtgagcgagcgagcgcgcag

Key:
Bold = 5’ ITR sequence; single underline = promoter sequence; italics = 5’ flanking sequence-guide sequence-microRNA loop sequence-passenger sequence-3’ flanking sequence; italics + wave underline: DNA encoding the GI passenger sequence; italics + bold + single underline: DNA encoding the GI guide sequence double underline: polyA sequence; bold + lowercase letters: 3’ ITR sequence (SEQ ID NO: 789).

The exemplary monocistronic, anti-Grik2 construct described above may include the Grik2 antisense guide sequence (e.g., GI, SEQ ID NO: 77) incorporated into an A-miR-scaffold, such that the microRNA coding sequence is a polynucleotide having the nucleic acid sequence of SEQ ID NO: 797 or is a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 797.

(SEQ ID NO: 797)
TCGACTAGGGATAACAGGGTAATTGTTTGAATGAGGCTTCAGTACTTTACAGAATCGTTGCCTGCAC
ATCTTGGAAACACTTGCTGGGATTACTTCTTCAGGTTAACCCAACAGAAGGCTCGAGAAGGTATATT
GCTGTTGACAGTGAGCGAC [symbol] TAGTGAAGCCACAGATG [symbol] [symbol]
CTGCCTACTGCCTCGGAATTCAAGGGGCTACTTTAGGAGCAATTATCTTGTTTAC
TAAAACTGAATACCTTGCTATCTCTTTGATACATTTTTACAAAGCTGAATTAAAATGGTATAAATTATCA
C

Key:
italics = 5’ flanking sequence-guide sequence-microRNA loop sequence-passenger sequence-3’ flanking sequence; italics + wave underline: DNA encoding the GI passenger sequence; italics + bold + underline: DNA encoding the GI guide sequence.

Another anti-Grik2 construct incorporating the GI anti-Grik2 sequence (SEQ ID NO: 77) may include an E-miR-scaffold, such that the microRNA coding sequence is a polynucleotide having the nucleic acid sequence of SEQ ID NO: 798 or is a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 798.

(SEQ ID NO: 798)
TCGACTAGGGATAACAGGGTAATTGTTTGAATGAGGCTTCAGTACTTTACAGAATCGTTGCCTGCAC
ATCTTGGAAACACTTGCTGGGATTACTTCTTCAGGTTAACCCAACAGAAGGCTAAAGAAGGTATATTG
CTGTTGACAGTGAGCGAC [symbol] CTGTGAAGCCACAGATGGG [symbol] [symbol]
GCTGCCTACTGCCTCGGACTTCAAGGGGCTACTTTAGGAGCAATTATCTTGTTT
ACTAAAACTGAATACCTTGCTATCTCTTTGATACATTTTTACAAAGCTGAATTAAAATGGTATAAATTAT
CAC

Key:
italics = 5’ flanking sequence-guide sequence-microRNA loop sequence-passenger sequence-3’ flanking sequence; italics + bold + single underline: DNA encoding the GI guide sequence; italics + wave underline: DNA encoding the GI passenger sequence.

Another monocistronic, anti-Grik2 construct of the disclosure is an AAV (e.g., AAV9) construct containing a hSyn promoter (SEQ ID NO: 790), ASO GI (SEQ ID NO: 77) incorporated into an E-miR-30 scaffold, and a stuffer sequence (SEQ ID NOs: 815 and 816). Such a construct may have the nucleic acid sequence of SEQ ID NO: 803 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 803 (see below). The expression construct of SEQ ID NO: 803 or a variant thereof may be incorporated into a vector having the nucleic acid sequence of SEQ ID NO: 804 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 804.

(SEQ ID NO: 803)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTAGCCCGGGCTAGGTCGACTCGACTAGGGATAACAGGG
TAATTGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAGCGA
CGTCTCGATATGGAGAACCCATGCTGTGAAGCCACAGATGGGCATGGGTTTTATATCGAGACGCT
GCCTACTGCCTCGGACTTCAAGGGGCTACTTTAGGAGCAATTATCTTGTTTACTAAAACTGAATAC
CTTGCTATCTCTTTGATACATTTTTACAAAGCTGAATTAAAATGGTATAAATTATCACGGGATCCGA
TCTTTTTCCCTGTGGGAAAAATTATGGGGACATGATGAAGCCCCTTGAGGATCTGAGTTGTGGGTAAT
AAAGGAAATTTATTTTCATTGCAATAGTGTGTTGGAATTTTTTGTGTCTCTCACTCGGCGGCCGCATA
GTCTATCCAGGTTGAGCATCCTGCTGGTGGTTACAAGAAACTGTTTGAAACTGTGGAGGAACTGTCC
TCGCCGCTCACAGCTCATGTAACAGGCAGGATCCCCCTCTGGCTCACCGGCAGTCTCCTTCGATGT
GGGCCAGGACTCTTTGAAGTTGGATCTGAGCCATTTTACCACCTGTTTGATGGGCAAGCCCTCCTGC
ACAAGTTTGACTTTAAAGAAGGACATGTCACATACCACAGAAGGTTCATCCGCACTGATGCTTACGTA
CGGGCAATGACTGAGAAAAGGATCGTCATAACAGAATTTGGCACCTGTGCTTTCCCAGATCCCTGCA
AGAATATATTTTCCAGGTTTTTTTCTTACTTTCGAGGAGTAGAGGTTACTGACAATTGCCCTTGTTAAT
GTCTACCCAGTGGGGGAAGATTACTACGCTTGCACAGAGACCAACTTTATTACAAAGATTAATCCAG
AGACCTTGGAGACAATTAAGCAGGTTGATCTTTGCAACTAAGTCTCTGTCAATGGGGCCACTGCTCA
CCCCCACATTGAAAATGATGGAACCGTTTACAATATTGGTAATTGCTTTGGAAAAAATTTTTCAATTGC
CTACAACATTGTAAAGATCCCACCACTGCAAGCAGACAAGGAAGATCCAATAAGCAAGTCAGAGATC
GTTGTACAATTCCCCTGCAGTGACCGATTCAAGCCATCTTACGTTCATAGTTTTGGTCTGACTCCCAA
CTATATCGTTTTTGTGGAGACACCAGTCAAAATTAACCTGTTCAAGTTCCTTTCTTCATGGAGTCTTTG
GGGAGCCAACTACATGGATTGTTTTGAGTCCAATGAAACCATGGGGTTTGGCTTCATATTGCTGACA
AAAAAAGGAAAAAGTACCTCAATAATAAATACAGAACTTCTCCTTTCAACCTCTTCCATCACATCAACA
CCTATGAAGACAATGGGTTTCTGATTGTGGATCTCTGCTGCTGGAAAGGATTTGAGTTTGTTTATAAT
TACTTATATTTAGCCAATTTACGTGAGAACTGGGAAGAGGTGAAAAAAAATGCCAGAAAGGCTCCCC
AACCTGAAGTTAGGAGATATGTACTTCCTTTGAATATTGACAAGGCTGACACAGGCAAGAATTTAGTC
AGCTCCCCAATACAACTGCCACTGCAATTCTGTGCAGTGACGAGACTATCTGGCTGGAGCCTGAAGT
TCTCTTTTCAGGGCCTCGTCAAGCATTTGAGTTTCCTCAAATCAATTACCAGAAGTATTGTGGGAAAC
CTTACACATATGCGTATGGACTTGGCTTGAATCACTTTGTTCCAGATAGGCTCTGTAAGCTGAATGTC
AAAACTAAAGAAACTTGGGTTTGGCAAGAGCCTGATTCATACCCATCAGAACCCATCTTTGTTTCTCA
CCCAGATGCCTTGGAAGAAGATGATGGTGTAGTTCTGAGTGTGGTGGTGAGCCCAGGAGCAGGACA
AAAGCCTGCTTATCTCCTGATTCTGAATGCCAAGGACTTAAGTGAAGTTGCCCGGGCTGAAGTGGAG
ATTAACATCCCTGTCACCTTTCATGGACTGTTCAAAAAATCTTGAccggccgccCGAGTTTAATTGGTTTA
TAGAACTCTTCA

Key:
single underline: promoter sequence; bold: microRNA stem-loop structure containing guide and passenger sequences; double underline: polyA sequence; italics: stutter sequence 1 (SEQ ID NO: 815); italics + underline: stutter sequence 2 (SEQ ID NO: 816).

In cases where the antisense construct contains the ASO sequence MW (SEQ ID NO: 80), the construct may include an E-miR-218-1 scaffold, such that the microRNA coding sequence is a polynucleotide having the nucleic acid sequence of SEQ ID NO: 799 or is a variant thereof having at least 15 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 799.

(SEQ ID NO: 799)
ACGTTTCCAGAACGTCTGTAGCTTTTCTCCTCCTTCCCTCCATTTTCCTCTTCCTCTTACCTTTCCCCT
AGTGGTTGGTGTAGTGATAATGTAGCGAGATTTTCTG [symbol] GGTTGCGA
GGTATGAGTAAA [symbol] TGGAACGTCACGCAGCTTTCTACAGCATGACAAG
CTGCTGAGGCTTAAATCAGGATTTTCCTGTCTCTTTCTACAAAATCAAAATGAAAAAAGAGGGCTTTTT
AGGCATCTCCGAGATTATGTG

Key:
italics = 5’ flanking sequence-guide sequence-microRNA loop sequence-passenger sequence-3’ flanking sequence; italics + bold + single underline: DNA encoding the GI guide sequence; italics + wave underline: DNA encoding the GI passenger sequence.

The construct of SEQ ID NO: 799 may further include an hSyn promoter (SEQ ID NO: 790) and a polyA sequence, as is shown below in SEQ ID NO: 819.

(SEQ ID NO: 819)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTGAATTCACGTTTCCAGAACGTCTGTAGCTTTTCTCCTCCT
TCCCTCCATTTTCCTCTTCCTCTTACCTTTCCCCTACTCCTTCCTCTACTCATAATCTACCCACATTT
TCTG [symbol] GGTTGCGAGGTATGAGTAAA [symbol] [symbol]
TGGAACGTCACGCAGCTTTCTACAGCATGACAAGCTGCTGAGGCTTAAATCAGGATTTTCCTGTCT
CTTTCTACAAAATCAAAATGAAAAAAGAGGGCTTTTTAGGCATCTCCGAGATTATGTGCTCGAGGGGA
TCCGATCTTTTTCCCTCTGCCAAAAATTATGGGGACATCATGAAGCCCCTTGAGCATCTGACTTCTGG
CTAATAAAGGAAATTTATTTTCATTGCAATAGTGTGTTGGAATTTTTTGTGTCTCTCACTCG

Key:
single underline = promoter sequence; italics = 5’ flanking sequence-guide sequence-microRNA loop sequence-passenger sequence-3’ flanking sequence; italics + wave underline: DNA encoding the GI passenger sequence; italics + bold + single underline: DNA encoding the GI guide sequence double underline: polyA sequence.

Another monocistronic, anti-Grik2 construct of the disclosure is an AAV (e.g., AAV9) constructs containing a hSyn promoter (SEQ ID NO: 790), ASO MW (SEQ ID NO: 80) incorporated into an E-miR-218-1 scaffold, and one or more stuffer sequences (e.g., SEQ ID NO: 815 and/or SEQ ID NO: 816). Such a construct may have the nucleic acid sequence of SEQ ID NO: 805 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 805 (see below). The expression construct of SEQ ID NO: 805 or a variant thereof may be incorporated into a vector having the nucleic acid sequence of SEQ ID NO: 806 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 806.

(SEQ ID NO: 805)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTGAATTCACGTTTCCAGAACGTCTGTAGCTTTTCTCCTCC
TTCCCTCCATTTTCCTCTTGGTCTTACCTTTGGCCTAGTGGTTGGTGTAGTGATAATGTAGCGAGAT
TTTCTGCAGAGCATTGCAGATGGACTGGGTTGCGAGGTATGAGTAAACAGTCCATACGCAATGCT
CCGTGGAACGTCACGCAGCTTTCTACAGCATGACAAGCTGCTGAGGCTTAAATCAGGATTTTCCTG
TCTCTTTCTACAAAATCAAAATGAAAAAAGAGGGCTTTTTAGGCATCTCCGAGATTATGTGCTCGA
GGGGATCCGATCTTTTTCCCTCTGCCAAAAATTATGGGGACATCATGAAGCCCCTTGAGCATCTGAC
TTCTGGCTAATAAAGGAAATTTATTTTCATTGCAATAGTGTGTTGGAATTTTTTGTGTCTCTCACTCGG
CGGCCGCATAGTCTATCCAGGTTGAGCATCCTGCTGGTGGTTACAAGAAACTGTTTGAAACTGTGGA
GGAACTGTCCTCGCCGCTCACAGCTCATGTAACAGGCAGGATCCCCCTCTGGCTCACCGGCAGTCT
CCTTCGATGTGGGCCAGGACTCTTTGAAGTTGGATCTGAGCCATTTTACCACCTGTTTGATGGGCAA
GCCCTCCTGCACAAGTTTGACTTTAAAGAAGGACATGTCACATACCACAGAAGGTTCATCCGCACTG
ATGCTTACGTACGGGCAATGACTGAGAAAAGGATCGTCATAACAGAATTTGGCACCTGTGCTTTCCC
AGATCCCTGCAAGAATATATTTTCCAGGTTTTTTTCTTACTTTCGAGGAGTAGAGGTTACTGACAATTG
CCCTTGTTAATGTCTACCCAGTGGGGGAAGATTACTACGCTTGCACAGAGACCAACTTTATTACAAA
GATTAATCCAGAGACCTTGGAGACAATTAAGCAGGTTGATCTTTGCAACTAAGTCTCTGTCAATGGG
GCCACTGCTCACCCCCACATTGAAAATGATGGAACCGTTTACAATATTGGTAATTGCTTTGGAAAAAA
TTTTTCAATTGCCTACAACATTGTAAAGATCCCACCACTGCAAGCAGACAAGGAAGATGGAATAAGGA
AGTCAGAGATCGTTGTACAATTCCCCTGCAGTGACCGATTCAAGCCATCTTACGTTCATAGTTTTGGT
CTGACTCCCAACTATATCGTTTTTGTGGAGACACCAGTCAAAATTAACCTGTTCAAGTTCCTTTCTTCA
TGGAGTCTTTGGGGAGCCAACTACATGGATTGTTTTGAGTCCAATGAAACCATGGGGTTTGGCTTCA
TATTGCTGACAAAAAAAGGAAAAAGTACCTCAATAATAAATACAGAACTTCTCCTTTCAACCTCTTCCA
TCACATCAACACCTATGAAGACAATGGGTTTCTGATTGTGGATCTCTGCTGCTGGAAAGGATTTGAGT
TTGTTTATAATTACTTATATTTAGCCAATTTACGTGAGAACTGGGAAGAGGTGAAAAAAAATGCCAGA
AAGGCTCCCCAACCTGAAGTTAGGAGATATGTACTTCCTTTGAATATTGACAAGGCTGACACAGGCA
AGAATTTAGTCAGCTCCCCAATACAACTGCCACTGCAATTCTGTGCAGTGACGAGACTATCTGGCTG
GAGCCTGAAGTTCTCTTTTCAGGGCCTCGTCAAGCATTTGAGTTTCCTCAAATCAATTACCAGAAGTA
TTGTGGGAAACCTTACACATATGCGTATGGACTTGGCTTGAATCACTTTGTTCCAGATAGGCTCTGTA
AGCTGAATGTCAAAACTAAAGAAACTTGGGTTTGGCAAGAGCCTGATTCATACCCATCAGAACCCAT
CTTTGTTTCTCACCCAGATGCCTTGGAAGAAGATGATGGTGTAGTTCTGAGTGTGGTGGTGAGCCCA
GGAGCAGGACAAAAGCCTGCTTATCTCCTGATTCTGAATGCCAAGGACTTAAGTGAAGTTGCCCGG
GCTGAAGTGGAGATTAACATCCCTGTCACCTTTCATGGACTGTTCAAAAAATCTTGAccggccgccCGAG
TTTAATTGGTTTATAGAACTCTTCA

Key:
single underline: promoter sequence; bold: microRNA stem-loop structure containing guide and passenger sequences; double underline: polyA sequence; italics: stutter sequence T (SEQ ID NO: 815); italics + underline: stutter sequence 2 (SEQ ID NO: 816).

Alternatively, the antisense construct containing the ASO sequence MW (SEQ ID NO: 80) may include an E-miR-124-3 scaffold, such that the microRNA coding sequence is a polynucleotide having the nucleic acid sequence of SEQ ID NO: 800 or is a variant thereof having at least 85% (at least 86%, 87%, 25 88%, 89%, 90%, 91%, 92%, 93%, 94%, 95%, 96%, 97%, 98%, 99%, or more) sequence identity to the nucleic acid sequence of SEQ ID NO: 800.

(SEQ ID NO: 800)
TCTGCCGCGGAAAGGGGAGAAGTGTGGGCTCCTCCGAGTCGGGGGCGGACTGGGACAGCACAGT
CGGCTGAGCGCAGCGCCCCCGCCCTGCCCGCCACGCGGCGAAGACGCCTGAGCGTTCGCGCCCC
TCGGGCGAGGACCCCACGCAAGCCCGAGCCGGTCCCGACCCTGGCCCCGACGCTCGCCGCCCGC
CCCAGCCCTGAGGGCCCCT [symbol] TTAATGTCTATACAAT [symbol] [symbol]
GAGAGGCGCCTCCGCCGCTCCTTTCTCATGGAAATGGCCCGCGAGCCCGT
CCGGCCCAGCGCCCCTCCCGCGGGAGGAAGGCGAGCCCGGCCCCCGGCGGCCATTCGCGCCGC
GGACAAATCCGGCGAACAATGCGCCCGCCCAGAGTGCGGCCCAGCTGCCGGGCCGGGGATCTGG
CCGCGGGACACAAAGGGGCCCGCACGCCTCTGGCGT

Key:
italics = 5’ flanking sequence-guide sequence-microRNA loop sequence-passenger sequence-3’ flanking sequence; italics + wave underline: DNA encoding the MW passenger sequence; italics + underline + gray highlight: DNA encoding the MW guide sequence.

The construct of SEQ ID NO: 800 may further include an hSyn promoter (SEQ ID NO: 790) and a polyA sequence, as is shown below in SEQ ID NO: 821.

(SEQ ID NO: 821)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTGAATTCTCTGCCGCGGAAAGGGGAGAAGTGTGGGCTCC
TCCGAGTCGGGGGCGGACTGGGACAGCACAGTCGGCTGAGCGCAGCGCCCCCGCCCTGCCCGCC
ACGCGGCGAAGACGCCTGAGCGTTCGCGCCCCTCGGGCGAGGACCCCACGCAAGCCCGAGCCGG
TCCCGACCCTGGCCCCGACGCTCGCCGCCCGCCCCAGCCCTGAGGGCCCCTCGA [symbol] [symbol]
TTAATGTCTATACAAT [symbol] GAGAGGCGCCTCCGCCG
CTCCTTTCTCATGGAAATGGCCCGCGAGCCCGTCCGGCCCAGCGCCCCTCCCGCGGGAGGAAGGC
GAGCCCGGCCCCCGGCGGCCATTCGCGCCGCGGACAAATCCGGCGAACAATGCGCCCGCCCAGA
GTGCGGCCCAGCTGCCGGGCCGGGGATCTGGCCGCGGGACACAAAGGGGCCCGCACGCCTCTGG
CGTCTCGAGGGGATCCGATCTTTTTCCCTCTGCCAAAAATTATGGGGACATCATGAAGCCCCTTGAG
CATCTGACTTCTGGCTAATAAAGGAAATTTATTTTCATTGCAATAGTGTGTTGGAATTTTTTGTGTCTC
TCACTCG

Key:
single underline = promoter sequence; italics = 5’ flanking sequence-guide sequence-microRNA loop sequence-passenger sequence-3’ flanking sequence; italics + wave underline: DNA encoding the GI passenger sequence; italics + bold + single underline: DNA encoding the GI guide sequence double underline: polyA sequence.

Another monocistronic, anti-Grik2 construct of the disclosure is an AAV (e.g., AAV9) constructs containing a hSyn promoter (SEQ ID NO: 790), ASO MW (SEQ ID NO: 80) incorporated into an E-miR-124-3 scaffold, and one or more stuffer sequences (SEQ ID NO: 815 and/or SEQ ID NO: 816). Such a construct may have the nucleic acid sequence of SEQ ID NO: 807 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 807 (see below). The expression construct of SEQ ID NO: 807 or a variant thereof may be incorporated into a vector having the nucleic acid sequence of SEQ ID NO: 808 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 808.

(SEQ ID NO: 807)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTGAATTCTCTGCCGCGGAAAGGGGAGAAGTGTGGGCTC
CTCCGAGTCGGGGGCGGACTGGGACAGCACAGTCGGCTGAGCGCAGCGCCCCCGCCCTGCCCG
CCACGCGGCGAAGACGCCTGAGCGTTCGCGCCCCTCGGGCGAGGACCCCACGCAAGCCCGAGC
CGGTCCCGACCCTGGCCCCGACGCTCGCCGCCCGCCCCAGCCCTGAGGGCCCCTCGACGTTTAT
CTACAACACTCTGATTTAATGTCTATACAATCAGAGCATTGCAGATGGACTGCGAGAGGCGCCTCC
GCCGCTCCTTTCTCATGGAAATGGCCCGCGAGCCCGTCCGGCCCAGCGCCCCTCCCGCGGGAGG
AAGGCGAGCCCGGCCCCCGGCGGCCATTCGCGCCGCGGACAAATCCGGCGAACAATGCGCCCG
CCCAGAGTGCGGCCCAGCTGCCGGGCCGGGGATCTGGCCGCGGGACACAAAGGGGCCCGCACG
CCTCTGGCGTCTCGAGGGGATCCGATGTTTTTCCCTCTGCCAAAAATTATGGGGACATCATGAAGCC
CCTTGAGGATCTGAGTTGTGGGTAATAAAGGAAATTTATTTTGATTGGAATAGTGTGTTGGAATTTTTT
GTGTCTCTCACTCGGCGGCCGCATAGTCTATCCAGGTTGAGCATCCTGCTGGTGGTTACAAGAAACT
GTTTGAAACTGTGGAGGAACTGTCCTCGCCGCTCACAGCTCATGTAACAGGCAGGATCCCCCTCTG
GCTCACCGGCAGTCTCCTTCGATGTGGGCCAGGACTCTTTGAAGTTGGATCTGAGCCATTTTACCAC
CTGTTTGATGGGCAAGCCCTCCTGCACAAGTTTGACTTTAAAGAAGGACATGTCACATACCACAGAA
GGTTCATCCGCACTGATGCTTACGTACGGGCAATGACTGAGAAAAGGATCGTCATAACAGAATTTGG
CACCTGTGCTTTCCCAGATCCCTGCAAGAATATATTTTCCAGGTTTTTTTCTTACTTTCGAGGAGTAG
AGGTTACTGACAATTGCCCTTGTTAATGTCTACCCAGTGGGGGAAGATTACTACGCTTGCACAGAGA
CCAACTTTATTACAAAGATTAATCCAGAGACCTTGGAGACAATTAAGCAGGTTGATCTTTGCAACTAA
GTCTCTGTCAATGGGGCCACTGCTCACCCCCACATTGAAAATGATGGAACCGTTTACAATATTGGTA
ATTGCTTTGGAAAAAATTTTTCAATTGCCTACAACATTGTAAAGATCCCACCACTGCAAGCAGACAAG
GAAGATCCAATAAGCAAGTCAGAGATCGTTGTACAATTCCCCTGCAGTGACCGATTCAAGCCATCTT
ACGTTCATAGTTTTGGTCTGACTCCCAACTATATCGTTTTTGTGGAGACACCAGTCAAAATTAACCTG
TTCAAGTTCCTTTCTTCATGGAGTCTTTGGGGAGCCAACTACATGGATTGTTTTGAGTCCAATGAAAC
CATGGGGTTTGGCTTCATATTGCTGACAAAAAAAGGAAAAAGTACCTCAATAATAAATACAGAACTTC
TCCTTTCAACCTCTTCCATCACATCAACACCTATGAAGACAATGGGTTTCTGATTGTGGATCTCTGCT
GCTGGAAAGGATTTGAGTTTGTTTATAATTACTTATATTTAGCCAATTTACGTGAGAACTGGGAAGAG
GTGAAAAAAAATGCCAGAAAGGCTCCCCAACCTGAAGTTAGGAGATATGTACTTCCTTTGAATATTGA
CAAGGCTGACACAGGCAAGAATTTAGTCAGCTCCCCAATACAACTGCCACTGCAATTCTGTGCAGTG
ACGAGACTATCTGGCTGGAGCCTGAAGTTCTCTTTTCAGGGCCTCGTCAAGCATTTGAGTTTCCTCA
AATCAATTACCAGAAGTATTGTGGGAAACCTTACACATATGCGTATGGACTTGGCTTGAATCACTTTG
TTCCAGATAGGCTCTGTAAGCTGAATGTCAAAACTAAAGAAACTTGGGTTTGGCAAGAGCCTGATTC
ATACCCATCAGAACCCATCTTTGTTTCTCACCCAGATGCCTTGGAAGAAGATGATGGTGTAGTTCTGA
GTGTGGTGGTGAGCCCAGGAGCAGGACAAAAGCCTGCTTATCTCCTGATTCTGAATGCCAAGGACT
TAAGTGAAGTTGCCCGGGCTGAAGTGGAGATTAACATCCCTGTCACCTTTCATGGACTGTTCAAAAA
ATCTTGAccggccgccCGAGTTTAATTGGTTTATAGAACTCTTCA

Key:
single underline: promoter sequence; bold: microRNA stem-loop structure containing guide and passenger sequences; double underline: polyA sequence; italics: stutter sequence 1 (SEQ ID NO: 815); italics + underline: stutter sequence 2 (SEQ ID NO: 816).

Multigene miRNA cassettes

The flank/stem-loop/flank construct (e.g., pri-miRNA) may be treated as a single miRNA “cassette” and can be concatenated (e.g., provided in a multi-gene arrangement driven by one or more promoters). More than one (e.g., at least 2, 3, 4, 5, 6, 7, 8, 9, 10, or more) pre-miR stem-loop sequence may be embedded in arbitrary polynucleotide sequences of a longer transcript (such as, e.g., an intron) or between endogenous microRNA flanking sequences (5′ and 3′ to each stem-loop, such as -5p and −3p sequences). Each pre-miR stem-loop sequence may be expressed under the control of a dedicated promoter (e.g., as a multi-gene construct with separate promoter sequences, each of which independently regulates the expression of an individual pre-miR stem-loop sequence; i.e., each promoter functions independent of the other to produce individual microRNAs). It has been shown that flanking sequences that can provide at least a 5-bp-extended stem were sufficient for the processing of the stem-loop (Sun, et al. Bio Techniques .41:59-63, July 2006, incorporated herein by reference). Spacer sequences may be positioned between the 3′ flanking sequence of a first miRNA expression cassette and the 5′ flanking sequence of a second miRNA expression cassette. Spacer sequences may be derived from coding or noncoding (e.g., intron) sequences and are of various lengths, but are not considered part of the stem-loop-flank sequence (Rousset, F. et al., Molecular Therapy: Nucleic Acids, 14:352-63, 2019, incorporated herein by reference.)

An exemplary expression cassette may include a nucleotide sequence containing: (a) a first polynucleotide encoding a first miRNA sequence containing a guide RNA sequence that hybridizes to a Grik2 mRNA; and (b) a second polynucleotide encoding a second miRNA sequence containing a guide

RNA sequence that hybridizes to a Grik2 mRNA. For example, the expression cassette may include, from 5′ to 3′: (a) a first 5′ flanking region located 5′ to a guide strand, said first flanking region that includes a first 5′ flanking sequence (e.g., any one of SEQ ID NOs: 752, 754, 756, 759, 762, 765, and 768 or a variant thereof having at least 85% (at least 86%, 87%, 88%, 89%, 90%, 91%, 92%, 93%, 94%, 95%, 96%, 97%, 98%, 99%, or more) sequence identity thereto); (b) a first stem-loop structure that includes: (i) a 5′ stem-loop arm that includes a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., SEQ ID NOs: 1-100); (ii) a loop region that includes a microRNA sequence selected from Table 6 or a variant thereof having at least 85% (at least 86%, 87%, 88%, 89%, 90%, 91%, 92%, 93%, 94%, 95%, 96%, 97%, 98%, 99%, or more) sequence identity thereto; (iii) a 3′ stem-loop arm that includes a passenger nucleotide sequence that is complementary or substantially complementary to the guide strand; (c) a first 3′ flanking region (e.g., any one of SEQ ID NOs: 753, 755, 757, 760, 763, 766, and 769 or a variant thereof having at least 85% (at least 86%, 87%, 88%, 89%, 90%, 91%, 92%, 93%, 94%, 95%, 96%, 97%, 98%, 99%, or more) sequence identity thereto) located 3′ to said passenger strand and a 3′ spacer sequence; (d) a second 5′ flanking region (e.g., any one of SEQ ID NOs: 752, 754, 756, 759, 762, 765, and 768 or a variant thereof having at least 85% (at least 86%, 87%, 88%, 89%, 90%, 91%, 92%, 93%, 94%, 95%, 96%, 97%, 98%, 99%, or more) sequence identity thereto) located 5′ to a guide strand; (e) a second stem-loop structure that includes: (i) a 5′ stem-loop arm that includes a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., SEQ ID NOs: 1-100); (ii) a loop region containing a microRNA sequence selected from Table 6 or a variant thereof having at least 85% (at least 86%, 87%, 88%, 89%, 90%, 91%, 92%, 93%, 94%, 95%, 96%, 97%, 98%, 99%, or more) sequence identity thereto; (iii) a 3′ stem-loop arm that includes a passenger nucleotide sequence complementary or substantially complementary to the guide strand; (f) a second 3′ flanking region that includes a 3′ flanking sequence (e.g., any one of SEQ ID NOs: 753, 755, 757, 760, 763, 766, and 769 or a variant thereof having at least 85% (at least 86%, 87%, 88%, 89%, 90%, 91%, 92%, 93%, 94%, 95%, 96%, 97%, 98%, 99%, or more) sequence identity thereto) located 3′ to the passenger strand.

The first 5′ flanking sequence, first 3′ flanking sequence, the second 5′ flanking sequence, and the second 3′ flanking sequence may be selected from Table 6.

Dual-miRNA, single promoter expression cassettes

A multigene or multi-gene rAAV expression construct may include a transgene made up of sequential (e.g., contiguous or non-contiguous) miRNA-encoding polynucleotides Xi, such as (Xi)n. The polynucleotide includes any one of the guide sequences listed in Table 2 and/or Table 3, a passenger sequence that is fully or substantially complementary to the guide sequence, any one of the 5′ and 3′ flanking sequences listed in Table 6, and any one of the loop sequences listed in Table 6. The multigene transgene having the formula, (Xi)n, is under control of a single promoter positioned at the 5′ end of the transgene such that the promoter and transgene have the formula, promoter-(k)n, where n is an integer from 1-(e.g., 1, 2, 3, 4, 5, 6, 7, 8 , 9, or 10).

A non-limiting example of a multi-microRNA construct of the disclosure includes a single promoter, e.g., an hSyn promoter (e.g., SEQ ID NO: 790), a GI antisense sequence (SEQ ID NO: 77) embedded in an endogenous (E)-miR-scaffold, and a MW antisense sequence (SEQ ID NO: 80) embedded in a E-miR-218-1 scaffold. Such a construct may have the nucleic acid sequence of SEQ ID NO: 811 or may be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 811 (see below). The construct of SEQ ID NO: 811 or a variant thereof may be incorporated into a vector having a nucleic acid sequence of SEQ ID NO: 812 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 812.

(SEQ ID NO: 811)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTAGCCCGGGCTAGGTCGAcTCGACTAGGGATAACAGGGT
AATTGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAGCGA
CGTCTCGATATGGAGAACCCATGCTGTGAAGCCACAGATGGGCATGGGTTTTATATCGAGACGCT
GCCTACTGCCTCGGACTTCAAGGGGCTACTTTAGGAGCAATTATCTTGTTTACTAAAACTGAATAC
CTTGCTATCTCTTTGATACATTTTTACAAAGCTGAATTAAAATGGTATAAATTATCACGGGATCCGA
ATTCACGTTTCCAGAACGTCTGTAGCTTTTCTCCTCCTTCCCTCCATTTTCCTCTTGGTCTTACCTTT
GGCCTAGTGGTTGGTGTAGTGATAATGTAGCGAGATTTTCTGCAGAGCATTGCAGATGGACTGGG
TTGCGAGGTATGAGTAAACAGTCCATACGCAATGCTCCGTGGAACGTCACGCAGCTTTCTACAGC
ATGACAAGCTGCTGAGGCTTAAATCAGGATTTTCCTGTCTCTTTCTACAAAATCAAAATGAAAAAA
GAGGGCTTTTTAGGCATCTCCGAGATTATGTGCTCGAGGGGATCCGATCTTTTTCCCTCTGCCAAAA
ATTATGGGGACATCATGAAGCCCCTTGAGCATCTGACTTCTGGCTAATAAAGGAAATTTATTTTCATT
GCAATAGTGTGTTGGAATTTTTTGTGTCTCTCACTCGGCGGCCGCATAGTCTATCCAGGTTGAGCAT
CCTGCTGGTGGTTACAAGAAACTGTTTGAAACTGTGGAGGAACTGTCCTCGCCGCTCACAGCTCATG
TAACAGGCAGGATCCCCCTCTGGCTCACCGGCAGTCTCCTTCGATGTGGGCCAGGACTCTTTGAAG
TTGGATCTGAGCCATTTTACCACCTGTTTGATGGGCAAGCCCTCCTGCACAAGTTTGACTTTAAAGAA
GGACATGTCACATACCACAGAAGGTTCATCCGCACTGATGCTTACGTACGGGCAATGACTGAGAAAA
GGATCGTCATAACAGAATTTGGCACCTGTGCTTTCCCAGATCCCTGCAAGAATATATTTTCCAGGTTT
TTTTCTTACTTTCGAGGAGTAGAGGTTACTGACAATTGCCCTTGTTAATGTCTACCCAGTGGGGGAAG
ATTACTACGCTTGCACAGAGACCAACTTTATTACAAAGATTAATCCAGAGACCTTGGAGACAATTAAG
CAGGTTGATCTTTGCAACTAAGTCTCTGTCAATGGGGCCACTGCTCACCCCCACATTGAAAATGATG
GAACCGTTTACAATATTGGTAATTGCTTTGGAAAAAATTTTTCAATTGCCTACAACATTGTAAAGATCC
CACCACTGCAAGCAGACAAGGAAGATCCAATAAGCAAGTCAGAGATCGTTGTACAATTCCCCTGCAG
TGACCGATTCAAGCCATCTTACGTTCATAGTTTTGGTCTGACTCCCAACTATAlCGTTTTTGTGGAGA
CACCAGTCAAAATTAACCTGTTCAAGTTCCTTTCTTCATGGAGTCTTTGGGGAGCCAACTACATGGAT
TGTTTTGAGTCCAATGAAACCATGGGGTTTGGCTTCATATTGCTGACAAAAAAAGGAAAAAGTACCTC
AATAATAAATACAGAACTTCTCCTTTCAACCTCTTCCATCACATCAACACCTATGAAGACAATGGGTTT
CTGATTGTGGATCTCTGCTGCTGGAAAGGATTTGAGTTTGTTTATAATTACTTATATTTAGCCAATTTA
CGTGAGAACTGGGAAGAGGTGAAAAAAAATGCCAGAAAGGCTCCCCAACCTGAAGTTAGGAGATAT
GTACTTCCTTTGAATATTGACAAGGCTGACACAGGCAAGAATTTAGTCAGCTCCCCAATACAACTGCC
ACTGCAATTCTGTGCAGTGACGAGACTATCTGGCTGGAGCCTGAAGTTCTCTTTTCAGGGCCTCGTC
AAGCATTTGAGTTTCCTCAAATCAATTACCAGAAGTATTGTGGGAAACCTTACACATATGCGTATGGA
CTTGGCTTGAATCACTTTGTTCCAGATAGGCTCTGTAAGCTGAATGTCAAAACTAAAGAAACTTGGGT
TTGGCAAGAGCCTGATTCATACCCATCAGAACCCATCTTTGTTTCTCACCCAGATGCCTTGGAAGAA
GATGATGGTGTAGTTCTGAGTGTGGTGGTGAGCCCAGGAGCAGGACAAAAGCCTGCTTATCTCCTG
ATTCTGAATGCCAAGGACTTAAGTGAAGTTGCCCGGGCTGAAGTGGAGATTAACATCCCTGTCACCT
TTCATGGACTGTTCAAAAAATCTTGA

Key:
single underline: promoter sequence; bold: microRNA stem-loop structure containing guide and passenger sequences; double underline: polyA sequence; italics: stutter sequence (SEQ ID NO: 815).

Dual-miRNA, Dual Promoter Expression Cassettes

A multi-gene expression cassette containing more than one (e.g., at least 2, 3, 4, 5, 6, 7, 8, 9, 10, or more) pre-miR stem-loop sequence may include more than one promoter sequence to regulate the expression of each individual pre-miR stem-loop sequence, such that each individual pre-miR stem-loop sequence is operably linked to a dedicated promoter sequence. In such cases, the expression construct features a structure of formula, (promoter-k)n, where X, is a polynucleotide containing any one of the guide sequences listed in Table 2 and/or Table 3, and n is an integer from 1-(e.g., 1, 2, 3, 4, 5, 6, 7, 8 , 9, or 10). Additional regulatory elements, such as enhancer sequences, terminator sequences, polyadenylation signals, introns, and/or sequences, capable of forming secondary structures, such as any one of the regulatory elements disclosed herein, may be operably linked to the 5′ end and/or the 3′ end of the promoter-X₁ structure.

In a particular example, the dual-miRNA expression cassette includes two pre-miR stem-loop sequences, each under control of an individual promoter sequence (e.g., a promoter sequence disclosed herein). The two promoters in the dual-miRNA cassette may be identical promoters or different promoters.

In a specific example, the dual-miRNA expression cassette includes a nucleotide sequence comprising, from 5′ to 3′: (a) a first promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (b) a first 5′ flanking region located 5′ to a first passenger nucleotide sequence (e.g., any one of SEQ ID NOs: 752, 754, 756, 759, 762, 765, or 768); (c) a first stem-loop sequence that includes, from 5′ to 3′: (i) a first 5′ stem-loop arm that includes the first passenger nucleotide sequence which is complementary or substantially complementary to a first guide sequence; (ii) a first loop region containing a first microRNA loop sequence (e.g., any one of SEQ ID NOs: 758, 761, 764, 767, or 770); (iii) a first 3′ stem-loop arm containing a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (d) a first 3′ flanking region located 3′ to the first guide nucleotide sequence (e.g., any one of SEQ ID NOs: 753, 755, 757, 760, 763, 766, or 769); (e) optionally, a second promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 30 88%, 89%, 90%, 91%, 92%, 93%, 94%, 95%, 96%, 97%, 98%, 99%, or more) sequence identity thereto); (f) a second 5′ flanking region located 5′ to a second passenger nucleotide sequence (e.g., any one of SEQ ID NOs: 752, 754, 756, 759, 762, 765, or 768); (g) a second stem-loop sequence that includes, from 5′ to 3′: (i) a second 5′ stem-loop arm containing the second passenger nucleotide sequence which is complementary or substantially complementary to a second guide sequence; (ii) a second loop region containing a second microRNA loop sequence (e.g., any one of SEQ ID NOs: 758, 761, 764, 767, or 770); (iii) a second 3′ stem-loop arm containing a second guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); and (h) a second 3′ flanking region located 3′ to the second guide nucleotide sequence (e.g., any one of SEQ ID NOs: 753, 755, 757, 760, 763, 766, or 769).

In another example, dual-miRNA the expression cassette includes a nucleotide sequence that includes, from 5′ to 3′: (a) a first promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (b) a first 5′ flanking region located 5′ to a first passenger nucleotide sequence (e.g., any one of SEQ ID NOs: 752, 754, 756, 759, 762, 765, or 768); (c) a first stem-loop sequence that includes, from 5′ to 3′: (i) a first 5′ stem-loop arm containing a first passenger nucleotide sequence which is complementary or substantially complementary to a first guide sequence; (ii) a first loop region containing a first microRNA loop sequence (e.g., any one of SEQ ID NOs: 758, 761, 764, 767, or 770); (iii) a first 3′ stem-loop arm containing a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (d) a first 3′ flanking region located 3′ to the first guide nucleotide sequence (e.g., any one of SEQ ID NOs: 753, 755, 757, 760, 763, 766, or 769); (e) optionally, a second promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (f) a second 5′ flanking region located 5′ to a second guide nucleotide sequence (e.g., any one of SEQ ID NOs: 752, 754, 756, 759, 762, 765, or 768); (g) a second stem-loop sequence that includes, from 5′ to 3′: (i) a second 5′ stem-loop arm containing a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a second loop region containing a second microRNA loop sequence (e.g., any one of SEQ ID NOs: 758, 761, 764, 767, or 770); (iii) a second 3′ stem-loop arm containing a second passenger nucleotide sequence which is complementary or substantially complementary to the second guide sequence; and (h) a second 3′ flanking region located 3′ to the second passenger nucleotide sequence (e.g., any one of SEQ ID NOs: 753, 755, 757, 760, 763, 766, or 769).

In another example, the dual-miRNA expression cassette includes a nucleotide sequence that includes, from 5′ to 3′: (a) a first promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (b) a first 5′ flanking region located 5′ to a first guide nucleotide sequence (e.g., any one of SEQ ID NOs: 752, 754, 756, 759, 762, 765, or 768); (c) a first stem-loop sequence that includes, from 5′ to 3′: (i) a first 5′ stem-loop arm containing a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a first loop region containing a first microRNA loop sequence (e.g., any one of SEQ ID NOs: 758, 761, 764, 767, or 770); (iii) a first 3′ stem-loop arm containing a first passenger nucleotide sequence which is complementary or substantially complementary to the first guide sequence; (d) a first 3′ flanking region located 3′ to the first passenger nucleotide sequence (e.g., any one of SEQ ID NOs: 753, 755, 757, 760, 763, 766, or 769); (e) optionally, a second promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (f) a second 5′ flanking region located 5′ to a second passenger nucleotide sequence (e.g., any one of SEQ ID NOs: 752, 754, 756, 759, 762, 765, or 768); (g) a second stem-loop sequence that includes, from 5′ to 3′: (i) a second 5′ stem-loop arm containing a second passenger nucleotide sequence which is complementary or substantially complementary to a second guide sequence; (ii) a second loop region containing a second microRNA loop sequence (e.g., any one of SEQ ID NOs: 758, 761, 764, 767, or 770); (iii) a second 3′ stem-loop arm containing a second guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); and (h) a second 3′ flanking region located 3′ to the second guide nucleotide sequence (e.g., any one of SEQ ID NOs: 753, 755, 757, 760, 763, 766, or 769).

In another example, the dual-miRNA expression cassette includes a nucleotide sequence that includes, from 5′ to 3′: (a) a first promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (b) a first 5′ flanking region located 5′ to a first guide nucleotide sequence (e.g., any one of SEQ ID NOs: 752, 754, 756, 759, 762, 765, or 768); (c) a first stem-loop sequence that includes, from 5′ to 3′: (i) a first 5′ stem-loop arm containing a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a first loop region containing a first microRNA loop sequence (e.g., any one of SEQ ID NOs: 758, 761, 764, 767, or 770); (iii) a first 3′ stem-loop arm containing a first passenger nucleotide sequence which is complementary or substantially complementary to the first guide sequence; (d) a first 3′ flanking region located 3′ to the first passenger nucleotide sequence (e.g., any one of SEQ ID NOs: 753, 755, 757, 760, 763, 766, or 769); (e) optionally, a second promoter sequence (e.g., any one of the promoter sequences disclosed herein, e.g., Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (f) a second 5′ flanking region located 5′ to a second guide nucleotide sequence (e.g., any one of SEQ ID NOs: 752, 754, 756, 759, 762, 765, or 768); (g) a second stem-loop sequence that includes, from 5′ to 3′: (i) a second 5′ stem-loop arm containing a guide nucleotide sequence having at least 85% (at least 86%, 87%, 10 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a second loop region containing a second microRNA loop sequence (e.g., any one of SEQ ID NOs: 758, 761, 764, 767, or 770); (iii) a second 3′ stem-loop arm containing a second passenger nucleotide sequence which is complementary or substantially complementary to the second guide sequence; and (h) a second 3′ flanking region located 3′ to the second passenger nucleotide sequence (e.g., any one of SEQ ID NOs: 753, 755, 757, 760, 763, 766, or 769).

In another example, the dual-miRNA expression cassette includes a nucleotide sequence that includes, from 5′ to 3′: (a) 5′ ITR sequence (e.g., polynucleotide having the nucleic acid sequence of SEQ ID NO: 746 or SEQ ID NO: 747 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 746 or SEQ ID NO: 747); (b) a first promoter sequence (e.g., any one of the promoter sequences disclosed herein Table or a variant thereof having at least 85% (at least 86%, 87%, 88%, 89%, 90%, 91%, 92%, 93%, 94%, 95%, 96%, 97%, 98%, 99%, or more) sequence identity to the promoter sequences listed in Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (c) a first 5′ flanking region located 5′ to a first passenger nucleotide sequence (e.g., any one of SEQ ID NOs: 752, 754, 756, 759, 762, 765, or 768 or a variant thereof having at least 85% (at least 86%, 87%, 88%, 89%, 90%, 91%, 92%, 93%, 94%, 95%, 96%, 97%, 98%, 99%, or more) sequence identity to any one of SEQ ID NOs: 752, 754, 756, 759, 762, 765, or 768); (d) a first stem-loop sequence that includes, from 5′ to 3′: (i) a first 5′ stem-loop arm containing a first passenger nucleotide sequence which is complementary or substantially complementary to the first guide sequence; (ii) a first loop region containing a first microRNA loop sequence (e.g., any one of SEQ ID NOs: 758, 761, 764, 767, or 770 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 758, 761, 764, 767, or 770); (iii) a first 3′ stem-loop arm containing a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (e) a first 3′ flanking region located 3′ to the first guide nucleotide sequence (e.g., any one of SEQ ID NOs: 753, 755, 757, 760, 763, 766, or 769 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 753, 755, 5 757, 760, 763, 766, or 769); (f) a BGH polyA sequence (e.g., SEQ ID NO: 793 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 793) (g) optionally, a second promoter sequence (e.g., any one of the promoter sequences disclosed herein Table or a variant thereof having at least 85% (at least 86%, 87%, 88%, 89%, 90%, 91%, 92%, 93%, 94%, 95%, 96%, 97%, 98%, 99%, or more) sequence identity to the promoter sequences listed in Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (h) a second 5′ flanking region located 5′ to a second passenger sequence (e.g., any one of SEQ ID NOs: 752, 754, 756, 759, 762, 765, or 768 or a variant thereof having at least 85% (at least 86%, 87%, 88%, 89%, 90%, 91%, 92%, 93%, 94%, 95%, 96%, 97%, 98%, 99%, or more) sequence identity to any one of SEQ ID NOs: 752, 754, 756, 759, 762, 765, or 768); (i) a second stem-loop sequence that includes, from 5′ to 3′: (i) a second 5′ stem-loop arm containing a second passenger nucleotide sequence which is complementary or substantially complementary to the second guide sequence; (ii) a second loop region containing a second microRNA loop sequence (e.g., any one of SEQ ID NOs: 758, 761, 764, 767, or 770 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 758, 761, 764, 767, or 770); (iii) a second 3′ stem-loop arm containing a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); and (j) a second 3′ flanking region located 3′ to the second guide nucleotide sequence (e.g., any one of SEQ ID NOs: 753, 755, 757, 760, 763, 766, or 769 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 753, 755, 757, 760, 763, 766, or 769); (k) an RBG polyA sequence (e.g., SEQ ID NO: 750, SEQ ID NO: 751, or SEQ ID NO: 792 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 750, SEQ ID NO: 751, or SEQ ID NO: 792); and a 3′ ITR sequence (e.g., SEQ ID NO: 748, SEQ ID NO: 749, or SEQ ID NO: 789 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 748, SEQ ID NO: 749, or SEQ ID NO: 789).

In another example, the dual-miRNA expression cassette includes a nucleotide sequence that includes, from 5′ to 3′: (a) 5′ ITR sequence (e.g., polynucleotide having the nucleic acid sequence of SEQ ID NO: 746 or SEQ ID NO: 747 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 746 or SEQ ID NO: 747); (b) a first promoter sequence (e.g., any one of the promoter sequences disclosed herein Table or a variant thereof having at least 85% (at least 86%, 87%, 88%, 89%, 90%, 91%, 92%, 93%, 94%, 95%, 96%, 97%, 98%, 99%, or more) sequence identity to the promoter sequences listed in Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (c) a first 5′ flanking region located 5′ to a first passenger nucleotide sequence (e.g., any one of SEQ ID NOs: 752, 754, 756, 759, 762, 765, or 768 or a variant thereof having at least 85% (at least 86%, 87%, 88%, 89%, 90%, 91%, 92%, 93%, 94%, 95%, 96%, 97%, 98%, 99%, or more) sequence identity to any one of SEQ ID NOs: 752, 754, 756, 759, 762, 765, or 768); (d) a first stem-loop sequence that includes, from 5′ to 3′: (i) a first 5′ stem-loop arm containing a first passenger nucleotide sequence which is complementary or substantially complementary to the first guide sequence; (ii) a first loop region containing a first microRNA loop sequence (e.g., any one of SEQ ID NOs: 758, 761, 764, 767, or 770 or a variant thereof having at least 85% (at least 86%, 87%, 88%, 89%, 90%, 91%, 92%, 93%, 94%, 95%, 96%, 97%, 98%, 15 99%, or more) sequence identity to SEQ ID NOs: 758, 761, 764, 767, or 770); (iii) a first 3′ stem-loop arm containing a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (e) a first 3′ flanking region located 3′ to the first guide nucleotide sequence (e.g., any one of SEQ ID NOs: 753, 755, 757, 760, 763, 766, or 769 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 753, 755, 757, 760, 763, 766, or 769); (f) a BGH polyA sequence (e.g., SEQ ID NO: 793 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 793) (g) optionally, a second promoter sequence (e.g., any one of the promoter sequences disclosed herein Table or a variant thereof having at least 85% (at least 86%, 87%, 88%, 89%, 90%, 91%, 92%, 93%, 94%, 95%, 96%, 97%, 98%, 99%, or more) sequence identity to the promoter sequences listed in Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (h) a second 5′ flanking region located 5′ to a second passenger sequence (e.g., any one of SEQ ID NOs: 752, 754, 756, 759, 762, 765, or 768 or a variant thereof having at least 85% (at least 86%, 87%, 88%, 89%, 90%, 91%, 92%, 93%, 94%, 95%, 96%, 97%, 98%, 99%, or more) sequence identity to any one of SEQ ID NOs: 752, 754, 756, 759, 762, 765, or 768); (i) a second stem-loop sequence that includes, from 5′ to 3′: (i) a second 5′ stem-loop arm containing a second passenger nucleotide sequence which is complementary or substantially complementary to the second guide sequence; (ii) a second loop region containing a second microRNA loop sequence (e.g., any one of SEQ ID NOs: 758, 761, 764, 767, or 770 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 758, 761, 764, 767, or 770); (iii) a second 3′ stem-loop arm containing a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); and (j) a second 3′ flanking region located 3′ to the second passenger nucleotide sequence (e.g., any one of SEQ ID NOs: 753, 755, 757, 760, 763, 766, or 769 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 753, 755, 757, 760, 763, 766, or 769); (k) an RBG polyA sequence (e.g., SEQ ID NO: 750, SEQ ID NO: 751, or SEQ ID NO: 792 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 750, SEQ ID NO: 751, or SEQ ID NO: 792); and a 3′ ITR sequence (e.g., SEQ ID NO: 748, SEQ ID NO: 749, or SEQ ID NO: 789 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 748, SEQ ID NO: 749, or SEQ ID NO: 789).

In another example, the dual-miRNA expression cassette includes a nucleotide sequence that includes, from 5′ to 3′: (a) 5′ ITR sequence (e.g., polynucleotide having the nucleic acid sequence of SEQ ID NO: 746 or SEQ ID NO: 747 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 746 or SEQ ID NO: 747); (b) a first promoter sequence (e.g., any one of the promoter sequences disclosed herein Table or a variant thereof having at least 85% (at least 86%, 87%, 88%, 89%, 90%, 91%, 92%, 93%, 94%, 95%, 96%, 97%, 98%, 99%, or more) sequence identity to the promoter sequences listed in Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 25 89%, 90%, 91%, 92%, 93%, 94%, 95%, 96%, 97%, 98%, 99%, or more) sequence identity thereto); (c) a first 5′ flanking region located 5′ to a first guide nucleotide sequence (e.g., any one of SEQ ID NOs: 752, 754, 756, 759, 762, 765, or 768 or a variant thereof having at least 85% (at least 86%, 87%, 88%, 89%, 90%, 91%, 92%, 93%, 94%, 95%, 96%, 97%, 98%, 99%, or more) sequence identity to any one of SEQ ID NOs: 752, 754, 756, 759, 762, 765, or 768); (d) a first stem-loop sequence that includes, from 5′ to 3′: (i) a first 5′ stem-loop arm containing a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a first loop region containing a first microRNA loop sequence (e.g., any one of SEQ ID NOs: 758, 761, 764, 767, or 770 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 758, 761, 764, 767, or 770); (iii) a first 3′ stem-loop arm containing a first passenger nucleotide sequence which is complementary or substantially complementary to the first guide sequence; (e) a first 3′ flanking region located 3′ to the first passenger nucleotide sequence (e.g., any one of SEQ ID NOs: 753, 755, 757, 760, 763, 766, or 769 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 753, 755, 757, 760, 763, 766, or 769); (f) a BGH polyA sequence (e.g., SEQ ID NO: 793 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 793) (g) optionally, a second promoter sequence (e.g., any one of the promoter sequences disclosed herein Table or a variant thereof having at least 5 85% (at least 86%, 87%, 88%, 89%, 90%, 91%, 92%, 93%, 94%, 95%, 96%, 97%, 98%, 99%, or more) sequence identity to the promoter sequences listed in Table 5, e.g., an hSyn promoter (e.g., any one of SEQ ID NOs: 682-685 and 790), CaMKII promoter (e.g., any one of SEQ ID NOs: 687-691 and 802), or C1ql2promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (h) a second 5′ flanking region located 5′ to a second passenger sequence (e.g., any one of SEQ ID NOs: 752, 754, 756, 759, 762, 765, or 768 or a variant thereof having at least 85% (at least 86%, 87%, 88%, 89%, 90%, 91%, 92%, 93%, 94%, 95%, 96%, 97%, 98%, 99%, or more) sequence identity to any one of SEQ ID NOs: 752, 754, 756, 759, 762, 765, or 768); (i) a second stem-loop sequence that includes, from 5′ to 3′: (i) a second 5′ stem-loop arm containing a second passenger nucleotide sequence which is complementary or substantially complementary to the second guide sequence; (ii) a second loop region containing a second microRNA loop sequence (e.g., any one of SEQ ID NOs: 758, 761, 764, 767, or 770 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 758, 761, 764, 767, or 770); (iii) a second 3′ stem-loop arm containing a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); and (j) a second 3′ flanking region located 3′ to the second guide nucleotide sequence (e.g., any one of SEQ ID NOs: 753, 755, 757, 760, 763, 766, or 769 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 753, 755, 757, 760, 763, 766, or 769); (k) an RBG polyA sequence (e.g., SEQ ID NO: 750, SEQ ID NO: 751, or SEQ ID NO: 792 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 750, SEQ ID NO: 751, or SEQ ID NO: 792); and a 3′ ITR sequence (e.g., SEQ ID NO: 748, SEQ ID NO: 749, or SEQ ID NO: 789 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 748, SEQ ID NO: 749, or SEQ ID NO: 789).

In another example, the dual-miRNA expression cassette includes a nucleotide sequence that includes, from 5′ to 3′: (a) 5′ ITR sequence (e.g., polynucleotide having the nucleic acid sequence of SEQ ID NO: 746 or SEQ ID NO: 747 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 746 or SEQ ID NO: 747); (b) a first promoter sequence (e.g., any one of the promoter sequences disclosed herein Table or a variant thereof having at least 85% (at least 86%, 87%, 88%, 89%, 90%, 91%, 92%, 93%, 94%, 95%, 96%, 97%, 98%, 99%, or more) sequence identity to the promoter sequences listed in Table 5, e.g., an hSyn promoter (e.g., any one of SEQ ID NOs: 682-685 and 790), CaMKII promoter (e.g., any one of SEQ ID NOs: 687-691 and 802), or C1ql2 promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (c) a first 5′ flanking region located 5′ to a first guide nucleotide sequence (e.g., any one of SEQ ID NOs: 752, 754, 756, 759, 762, 765, or 768 or a variant thereof having at least 85% (at least 86%, 87%, 88%, 89%, 90%, 91%, 92%, 93%, 94%, 95%, 96%, 97%, 98%, 99%, or more) sequence identity to any one of SEQ ID NOs: 752, 754, 756, 759, 762, 765, or 768); (d) a first stem-loop sequence that includes, from 5′ to 3′: (i) a first 5′ stem-loop arm containing a first guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a first loop region containing a first microRNA loop sequence (e.g., any one of SEQ ID NOs: 758, 761, 764, 767, or 770 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 758, 761, 764, 767, or 770); (iii) a first 3′ stem-loop arm containing a first passenger nucleotide sequence which is complementary or substantially complementary to the first guide sequence; (e) a first 3′ flanking region located 3′ to the first passenger nucleotide sequence (e.g., any one of SEQ ID NOs: 753, 755, 757, 760, 763, 766, or 769 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 753, 755, 757, 760, 763, 766, or 769); (f) a BGH polyA sequence (e.g., SEQ ID NO: 793 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 793) (g) optionally, a second promoter sequence (e.g., any one of the promoter sequences disclosed herein Table or a variant thereof having at least 85% (at least 86%, 87%, 88%, 89%, 90%, 91%, 92%, 93%, 94%, 95%, 96%, 97%, 98%, 99%, or more) sequence identity to the promoter sequences listed in Table 5, e.g., an hSyn promoter (e.g., any one of SEQ ID NOs: 682-685 and 790), CaMKII promoter (e.g., any one of SEQ ID NOs: 687-691 and 802), or C1ql2promoter (e.g., SEQ ID NO: 719 or SEQ ID NO: 791) or a variant thereof with at least 85% (at least 86%, 87%, 88%, 89%, 90%, 91%, 92%, 93%, 94%, 95%, 96%, 97%, 98%, 99%, or more) sequence identity thereto); (h) a second 5′ flanking region located 5′ to a second guide nucleotide sequence (e.g., any one of SEQ ID NOs: 752, 754, 756, 759, 762, 765, or 768 or a variant thereof having at least 85% (at least 86%, 87%, 88%, 89%, 90%, 91%, 92%, 93%, 94%, 95%, 96%, 97%, 98%, 99%, or more) sequence identity to any one of SEQ ID NOs: 752, 754, 756, 759, 762, 765, or 768); (i) a second stem-loop sequence that includes, from 5′ to 3′: (i) a second 5′ stem-loop arm containing a guide nucleotide sequence having at least 85% (at least 86%, 87%, 88%, 89%, 90%, 91%, 92%, 93%, 94%, 95%, 96%, 97%, 98%, 99%, or more) sequence identity to any one of the guide sequences listed in Table 2 and/or Table 3 (e.g., G9 (SEQ ID NO: 68), GI (SEQ ID NO: 77), MW (SEQ ID NO: 80), or MU (SEQ ID NO: 96) or a variant thereof with at least 85% (at least 86%, 87%, 88%, 89%, 90%, 91%, 92%, 93%, 94%, 95%, 96%, 97%, 98%, 99%, or more) sequence identity thereto); (ii) a second loop region containing a second microRNA loop sequence (e.g., any one of SEQ ID NOs: 758, 761, 764, 767, or 770 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 758, 761, 764, 767, or 770); (iii) a second 3′ stem-loop arm containing a second passenger nucleotide sequence which is complementary or substantially complementary to the second guide sequence; and (j) a second 3′ flanking region located 3′ to the second passenger nucleotide sequence (e.g., any one of SEQ ID NOs: 753, 755, 757, 760, 763, 766, or 769 or a variant thereof having at least 85% (at least 86%, 87%, 88%, 89%, 90%, 91%, 92%, 93%, 94%, 95%, 96%, 97%, 98%, 99%, or more) sequence identity to SEQ ID NOs: 753, 755, 757, 760, 763, 766, or 769); (k) an RBG polyA sequence (e.g., SEQ ID NO: 750, SEQ ID NO: 751, or SEQ ID NO: 792 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 750, SEQ ID NO: 751, or SEQ ID NO: 792); and a 3′ ITR sequence (e.g., SEQ ID NO: 748, SEQ ID NO: 749, or SEQ ID NO: 789 or a variant thereof having at least 85% (at least 86%, 87%, 88%, 89%, 90%, 91%, 92%, 93%, 94%, 95%, 96%, 97%, 98%, 99%, or more) sequence identity to SEQ ID NO: 748, SEQ ID NO: 749, or SEQ ID NO: 789).

In one example, the first guide sequence is a G9 sequence (SEQ ID NO: 68) or a variant thereof with at least 85% (at least 86%, 87%, 88%, 89%, 90%, 91%, 92%, 93%, 94%, 95%, 96%, 97%, 98%, 99%, or more) sequence identity thereto and the second guide sequence is a GI sequence (SEQ ID NO: 77) or a variant thereof with at least 85% (at least 86%, 87%, 88%, 89%, 90%, 91%, 92%, 93%, 94%, 95%, 96%, 97%, 98%, 99%, or more) sequence identity thereto. In another example, the first guide sequence is a G9 sequence (SEQ ID NO: 68 or a variant thereof with at least 85% (at least 86%, 87%, 88%, 89%, 90%, 91%, 92%, 93%, 94%, 95%, 96%, 97%, 98%, 99%, or more) sequence identity thereto and the second guide sequence is a MW sequence (SEQ ID NO: 80) or a variant thereof with at least 85% (at least 86%, 87%, 88%, 89%, 90%, 91%, 92%, 93%, 94%, 95%, 96%, 97%, 98%, 99%, or more) sequence identity thereto. In another example, the first guide sequence is a GI sequence (SEQ ID NO: 77) or a variant thereof with at least 85% (at least 86%, 87%, 88%, 89%, 90%, 91%, 92%, 93%, 94%, 95%, 96%, 97%, 98%, 99%, or more) sequence identity thereto and the second guide sequence is a G9 sequence (SEQ ID NO: 68) or a variant thereof with at least 85% (at least 86%, 87%, 88%, 89%, 90%, 91%, 92%, 93%, 94%, 95%, 96%, 97%, 98%, 99%, or more) sequence identity thereto. In yet another example, the first guide sequence is a GI sequence (SEQ ID NO: 77) or a variant thereof with at least 85% (at least 86%, 87%, 88%, 89%, 90%, 91%, 92%, 93%, 94%, 95%, 96%, 97%, 98%, 99%, or more) sequence identity thereto and the second guide sequence is a MW sequence (SEQ ID NO: 80) or a variant thereof with at least 85% (at least 86%, 87%, 88%, 89%, 90%, 91%, 92%, 93%, 94%, 95%, 96%, 97%, 98%, 99%, or more) sequence identity thereto. In another example, the first promoter is a SYN promoter (e.g., any one of SEQ ID NOs: 682-685 and 790 or a variant thereof having at least 85% (e.g., at least 86%, 90%, 95%, 96%, 97%, 98%, 99%, or more) sequence identity thereto and, optionally, the second promoter is a CAMKII promoter (e.g., any one of SEQ ID NOs: 687-691 and 802 or a variant thereof having at least 85% (e.g., at least 86%, 90%, 95%, 96%, 97%, 98%, 99%, or more) sequence identity thereto.

Sequence identity for the sequences described in the aforementioned dual-miRNA expression cassettes may be determined over a range of 10-1500 (e.g., 20-1400, 30-1300, 40-1200, 50-1100, 60-1000, 70-900, 80-800, 90-700, 100-600, 200-500, or 300-400) nucleotides. For example, sequence identity to the aforementioned dual-miRNA expression cassettes may be determined over nucleotides. In another example, sequence identity to the aforementioned dual-miRNA expression cassettes is determined over nucleotides. In another example, sequence identity to the aforementioned dual-miRNA expression cassettes is determined over nucleotides. In another example, sequence identity to the aforementioned dual-miRNA expression cassettes is determined over nucleotides. In another example, sequence identity to the aforementioned dual-miRNA expression cassettes is determined over 50 nucleotides. In another example, sequence identity to the aforementioned dual-miRNA expression cassettes is determined over 60 nucleotides. In another example, sequence identity to the aforementioned dual-miRNA expression cassettes is determined over 70 nucleotides. In another example, sequence identity to the aforementioned dual-miRNA expression cassettes is determined over 80 nucleotides. In another example, sequence identity to the aforementioned dual-miRNA expression cassettes is determined over 90 nucleotides. In another example, sequence identity to the aforementioned dual-miRNA expression cassettes is determined over 100 nucleotides. In another example, sequence identity to the aforementioned dual-miRNA expression cassettes is determined over 10 150 nucleotides. In another example, sequence identity to the aforementioned dual-miRNA expression cassettes is determined over 200 nucleotides. In another example, sequence identity to the aforementioned dual-miRNA expression cassettes is determined over 250 nucleotides. In another example, sequence identity to the aforementioned dual-miRNA expression cassettes is determined over 300 nucleotides. In another example, sequence identity to the aforementioned dual-miRNA expression cassettes is determined over 350 nucleotides. In another example, sequence identity to the aforementioned dual-miRNA expression cassettes is determined over 400 nucleotides. In another example, sequence identity to the aforementioned dual-miRNA expression cassettes is determined over 450 nucleotides. In another example, sequence identity to the aforementioned dual-miRNA expression cassettes is determined over 500 nucleotides. In another example, sequence identity to the aforementioned dual-miRNA expression cassettes is determined over 550 nucleotides. In another example, sequence identity to the aforementioned dual-miRNA expression cassettes is determined over 600 nucleotides. In another example, sequence identity to the aforementioned dual-miRNA expression cassettes is determined over 650 nucleotides. In another example, sequence identity to the aforementioned dual-miRNA expression cassettes is determined over 700 nucleotides. In another example, sequence identity to the aforementioned dual-miRNA expression cassettes is determined over 750 nucleotides. In another example, sequence identity to the aforementioned dual-miRNA expression cassettes is determined over 800 nucleotides. In another example, sequence identity to the aforementioned dual-miRNA expression cassettes is determined over 850 nucleotides. In another example, sequence identity to the aforementioned dual-miRNA expression cassettes is determined over 30 900 nucleotides. In another example, sequence identity to the aforementioned dual-miRNA expression cassettes is determined over 950 nucleotides. In another example, sequence identity to the aforementioned dual-miRNA expression cassettes is determined over 1000 nucleotides. In another example, sequence identity to the aforementioned dual-miRNA expression cassettes is determined over 1100 nucleotides. In another example, sequence identity to the aforementioned dual-miRNA expression cassettes is determined over 1200 nucleotides. In another example, sequence identity to the aforementioned dual-miRNA expression cassettes is determined over 1300 nucleotides. In another example, sequence identity to the aforementioned dual-miRNA expression cassettes is determined over 1400 nucleotides. In yet another example, sequence identity to the aforementioned dual-miRNA expression cassettes is determined over 1500 nucleotides. The dual-miRNA expression cassettes described above may include a promoter that is selected from the group consisting of a a U6 promoter, H1 promoter, 7SK promoter, Apolipoprotein E-Human Alpha 1-Antitrypsin promoter, CAG promoter, CBA promoter, CK8 promoter, mU1 a promoter, Elongation Factor 1α promoter, Thyroxine Binding Globulin promoter, Synapsin promoter, RNA Binding Fox-1 Homolog 3 promoter, Calcium/Calmodulin Dependent Protein Kinase II promoter, neuron-specific enolase promoter, Platelet Derived Growth Factor Subunit β, Vesicular Glutamate Transporter promoter, Somatostatin promoter, Neuropeptide Y promoter, Vasoactive Intestinal Peptide promoter, Parvalbumin promoter, Glutamate Decarboxylase 65 promoter, Glutamate Decarboxylase 67 promoter, Dopamine Receptor D1 promoter, Dopamine Receptor D2 promoter, Complement C1q Like 2 promoter, Proopiomelanocortin promoter, Prospero Homeobox 1 promoter, Microtubule Associated Protein 1B promoter, and Tubulin Alpha 1 promoter.

MicroRNA loop sequences suitable for use in conjunction with the dual-miRNA expression cassette disclosed herein may be a miR-30, miR-155, miR-218-1, or miR-124-3 loop sequence.

Dual-miRNA expression cassettes of the disclosure may also incorporate a 5′-ITR (e.g., SEQ ID NO: 746 or SEQ ID NO: 747) on the 5′ end of the expression cassette and a 3′-ITR (e.g., any one of SEQ ID NO: 748, 749, and 789) on the 3′ end of the expression cassette.

Moreover, the dual-miRNA expression constructs disclosed herein may include a first polyadenylation (polyA) signal operably linked between the 3′ end of the first 3′ flanking region and the 5′ end of the second promoter and/or a second polyA signal operably linked between the second 3′ end of the second 3′ flanking region and the 3′ ITR. The first polyA signal and the second polyA signal may be identical (e.g., both are RBG or BGH polyA signals) or different (e.g., the first polyA signal is RBG polyA and second polyA signal is BGH polyA; or the first polyA signal is BGH polyA and the second polyA signal is RBG polyA).

An exemplary dual-miRNA, anti-Grik2 construct of the disclosure may include an AAV (e.g., AAV9) construct containing an hSyn promoter (SEQ ID NO: 790) operably linked to a G9 (SEQ ID NO: 68) ASO sequence incorporated into an A-miR-scaffold followed by a CaMKII promoter (SEQ ID NO: 802) operably linked to a GI (SEQ ID NO: 77) ASO sequence incorporated into an A-miR-scaffold (DMTPV1). Such a construct may have the nucleic acid sequence of SEQ ID NO: 785 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 785 (see below).

(SEQ ID NO: 785)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGATCCGGT
CGGGCCCGCGGTACCGTCGAGAAGCTTGATGTGGGCGGAGCTTCGAAGGGGCGGGCGCCCGTGG
GGCGGGTCCTGAGTGGGGGCGGGACCGGGGCCGGCACCTGGGTGAGGTTCTGCAGAGGGCCCTG
CGTATGAGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCAGAGAGGG
GGAGGGGAAACAGGATGCGGCGAGGCGCGTGCGCACTGCCAGCTTCAGCACCGCGGACAGTGCC
TTCGCCCCCGCCTGGCGGCGCGCGCCACCGCCGCCTCAGCACTGAAGGCGCGCTGACGTCACTC
GCCGGTCCCCCGCAAACTCCCCTTCCCGGCCACCTTGGTCGCGTCCGCGCCGCCGCCGGCCCAGC
CGGACCGCACCACGCGAGGCGCGAGATAGGGGGGCACGGGCGCGACCATCTGCGCTGCGGCGCC
GGCGACTCAGCGCTGCCTCAGTCTGCGGTGGGCAGCGGAGGAGTCGTGTCGTGCCTGAGAGCGCA
GGGCGCGCCTGAATTCTCTGCCGCGGAAAGGGGAGAAGTGTGGGCTCCTCCGAGTCGGGGGCGG
ACTGGGACAGCACAGTCGGCTGAGCGCAGCGCCCCCGCCCTGCCCGCCACGCGGCGAAGACGCC
TGAGCGTTCGCGCCCCTCGGGCGAGGACCCCACGCAAGCCCGAGCCGGTCCCGACCCTGGCCCC
GACGCTCGCCGCCCGCCCCAGCCCTGAGGGCCCCTCTA [symbol] TTAATG
TCTATACAAT [symbol] GAGAGGCGCCTCCGCCGCTCCTTTCTCATGGAAAT
GGCCCGCGAGCCCGTCCGGCCCAGCGCCCCTCCCGCGGGAGGAAGGCGAGCCCGGCCCCCGGC
GGCCATTCGCGCCGCGGACAAATCCGGCGAACAATGCGCCCGCCCAGAGTGCGGCCCAGCTGCC
GGGCCGGGGATCTGGCCGCGGGACACAAAGGGGCCCGCACGCCTCTGGCGTCTCGAGGGGATCC
CTGTGCCTTCTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAGACAATAGCAGGCAT
GCTGGGGAGGCGGCCGCCCGAGTTTAATTGGTTTATAGAACTCTTCAAGCTAGCACTAGTGAAGCA
ATTCGTTGCATTATGGCCTTAGGTCACTTCATCTCCATGGGGTTCTTCTTCTGATTTTCTAGAAAATGA
GATGGGGGTGCAGAGAGCTTCCTCAGTGACCTGCCCAGGGTCACATCAGAAATGTCAGAGCTAGAA
CTTGAACTCAGATTACTAATCTTAAATTCCATGCCTTGGGGGCATGCAAGTACGATATACAGAAGGAG
TGAACTCATTAGGGCAGATGACCAATGAGTTTAGGAAAGAAGAGTCCAGGGCAGGGTACATCTACAC
CACCCGCCCAGCCCTGGGTGAGTCCAGCCACGTTCACCTCATTATAGTTGCCTCTCTCCAGTCCTAC
CTTGACGGGAAGCACAAGCAGAAACTGGGACAGGAGCCCCAGGAGACCAAATCTTCATGGTCCCTC
TGGGAGGATGGGTGGGGAGAGCTGTGGCAGAGGCCTCAGGAGGGGCCCTGCTGCTCAGTGGTGA
CAGATAGGGGTGAGAAAGCAGACAGAGTCATTCCGTCAGCATTCTGGGTCTGTTTGGTACTTCTTCT
CACGCTAAGGTGGCGGTGTGATATGCACAATGGCTAAAAAGCAGGGAGAGCTGGAAAGAAACAAGG
ACAGAGACAGAGGCCAAGTCAACCAGACCAATTCCCAGAGGAAGCAAAGAAACCATTACAGAGACT
ACAAGGGGGAAGGGAAGGAGAGATGAATTAGCTTCCCCTGTAAACCTTAGAACCCAGCTGTTGCCA
GGGCAACGGGGCAATACCTGTCTCTTCAGAGGAGATGAAGTTGCCAGGGTAACTACATCCTGTCTTT
CTCAAGGACCATCCCAGAATGTGGCACCCACTAGCCGTTACCATAGCAACTGCCTCTTTGCCCCACT
TAATCCCATCCCGTCTGTTAAAAGGGCCCTATAGTTGGAGGTGGGGGAGGTAGGAAGAGCGATGAT
CACTTGTGGACTAAGTTTGTTCGCATCCCCTTCTCCAACCCCCTCAGTACATCACCCTGGGGGAACA
GGGTCCACTTGCTCCTGGGCCCACACAGTCCTGCAGTATTGTGTATATAAGGCCAGGGCAAAGAGG
AGCAGGTTTTAAAGTGAAAGGCAGGCAGGTGTTGGGGAGGCAGTTACCGGGGCAACGGGAACAGG
GCGTTTCGGAGGTGGTTGCCATGGGGACCTGGATGCTGACGAAGGCTCGCGAGGCTGTGAGCAGC
CACAGTGCCCTGCTCAGAAGCCCCAAGCTCGTCAGTCAAGCCGGTTCTCCGTTTGCACTCAGGAGC
ACGGGCAGGCGAGTGGCCCCTAGTTCTGGGGGCAGCGAATTCCAATTGGCGCGCCTAGCCCGGGC
TAGGTCGACTCGACTAGGGATAACAGGGTAATTGTTTGAATGAGGCTTCAGTACTTTACAGAATCGTT
GCCTGCACATCTTGGAAACACTTGCTGGGATTACTTCTTCAGGTTAACCCAACAGAAGGCTAAAGAA
GGTATATTGCTGTTGACAGTGAGCGAC [symbol] GCTGTGAAGCCACAGATG
GG [symbol] GCTGCCTACTGCCTCGGACTTCAAGGGGCTACTTTAGGAGCAATT
ATCTTGTTTACTAAAACTGAATACCTTGCTATCTCTTTGATACATTTTTACAAAGCTGAATTAAAATGGT
ATAAATTATCACACCGGTCTCGAGGGATCCGATCTTTTTCCCTCTGCCAAAAATTATGGGGACATCAT
GAAGCCCCTTGAGCATCTGACTTCTGGCTAATAAAGGAAATTTATTTTCATTGCAATAGTGTGTTGGA
ATTTTTTGTGTCTCTCACTCGGCGGCCGCCCGAGTTTAATTGGTTTATAGAACTCTTCAAGCTAGCGA
AGCAATTCGTTGATCTGAATTTCGACCACCCATAATACCCATTACCCTGGTAGATAAGTAGCATGGCG
GGTTAATCATTAACTACAaggaacccctagtgatggagttggccactccctctctgcgcgctcgctcgctcactgaggccggg
cgaccaaaggtcgcccgacgcccgggctttgcccgggcggcctcagtgagcgagcgagcgcgcag

Key:
Bold = 5’ ITR sequence; single underline = promoter sequence; italics = 5’ flanking sequence-guide sequence-microRNA loop sequence-passenger sequence-3’ flanking sequence; italics + wave underline: DNA encoding the passenger sequence complementary to a guide sequence (G9 and GI-in that order); italics + bold + single underline: DNA encoding the guide sequence (G9 and GI-in that order); double underline: polyA sequence; bold + lowercase letters: 3’ ITR sequence (SEQ ID NO: 789).

Another exemplary dual-miRNA, anti-Grik2 construct of the disclosure may include an AAV (e.g., AAV9) construct containing an hSyn promoter (SEQ ID NO: 790) operably linked to a G9 (SEQ ID NO: 68) ASO sequence incorporated into an E-miR-124-3 scaffold followed by a CaMKII promoter (SEQ ID NO: 802) operably linked to a MW (SEQ ID NO: 80) ASO sequence incorporated into an E-miR-218 scaffold (DMTPV2). Such a construct may have the nucleic acid sequence of SEQ ID NO: 786 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 786 (see below).

(SEQ ID NO: 786)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGATCCGGT
CGGGCCCGCGGTACCGTCGAGAAGCTTGATGTGGGCGGAGCTTCGAAGGGGCGGGCGCCCGTGG
GGCGGGTCCTGAGTGGGGGCGGGACCGGGGCCGGCACCTGGGTGAGGTTCTGCAGAGGGCCCTG
CGTATGAGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCAGAGAGGG
GGAGGGGAAACAGGATGCGGCGAGGCGCGTGCGCACTGCCAGCTTCAGCACCGCGGACAGTGCC
TTCGCCCCCGCCTGGCGGCGCGCGCCACCGCCGCCTCAGCACTGAAGGCGCGCTGACGTCACTC
GCCGGTCCCCCGCAAACTCCCCTTCCCGGCCACCTTGGTCGCGTCCGCGCCGCCGCCGGCCCAGC
CGGACCGCACCACGCGAGGCGCGAGATAGGGGGGCACGGGCGCGACCATCTGCGCTGCGGCGCC
GGCGACTCAGCGCTGCCTCAGTCTGCGGTGGGCAGCGGAGGAGTCGTGTCGTGCCTGAGAGCGCA
GGGCGCGCCTGAATTCTCTGCCGCGGAAAGGGGAGAAGTGTGGGCTCCTCCGAGTCGGGGGCGG
ACTGGGACAGCACAGTCGGCTGAGCGCAGCGCCCCCGCCCTGCCCGCCACGCGGCGAAGACGCC
TGAGCGTTCGCGCCCCTCGGGCGAGGACCCCACGCAAGCCCGAGCCGGTCCCGACCCTGGCCCC
GACGCTCGCCGCCCGCCCCAGCCCTGAGGGCCCCTCTA [symbol] TTAATG
TCTATACAAT [symbol] GAGAGGCGCCTCCGCCGCTCCTTTCTCATGGAAAT
GGCCCGCGAGCCCGTCCGGCCCAGCGCCCCTCCCGCGGGAGGAAGGCGAGCCCGGCCCCCGGC
GGCCATTCGCGCCGCGGACAAATCCGGCGAACAATGCGCCCGCCCAGAGTGCGGCCCAGCTGCC
GGGCCGGGGATCTGGCCGCGGGACACAAAGGGGCCCGCACGCCTCTGGCGTCTCGAGGGGATCC
CTGTGCCTTCTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAGACAATAGCAGGCAT
GCTGGGGAGGCGGCCGCCCGAGTTTAATTGGTTTATAGAACTCTTCAAGCTAGCACTAGTGAAGCA
ATTCGTTGCATTATGGCCTTAGGTCACTTCATCTCCATGGGGTTCTTCTTCTGATTTTCTAGAAAATGA
GATGGGGGTGCAGAGAGCTTCCTCAGTGACCTGCCCAGGGTCACATCAGAAATGTCAGAGCTAGAA
CTTGAACTCAGATTACTAATCTTAAATTCCATGCCTTGGGGGCATGCAAGTACGATATACAGAAGGAG
TGAACTCATTAGGGCAGATGACCAATGAGTTTAGGAAAGAAGAGTCCAGGGCAGGGTACATCTACAC
CACCCGCCCAGCCCTGGGTGAGTCCAGCCACGTTCACCTCATTATAGTTGCCTCTCTCCAGTCCTAC
CTTGACGGGAAGCACAAGCAGAAACTGGGACAGGAGCCCCAGGAGACCAAATCTTCATGGTCCCTC
TGGGAGGATGGGTGGGGAGAGCTGTGGCAGAGGCCTCAGGAGGGGCCCTGCTGCTCAGTGGTGA
CAGATAGGGGTGAGAAAGCAGACAGAGTCATTCCGTCAGCATTCTGGGTCTGTTTGGTACTTCTTCT
CACGCTAAGGTGGCGGTGTGATATGCACAATGGCTAAAAAGCAGGGAGAGCTGGAAAGAAACAAGG
ACAGAGACAGAGGCCAAGTCAACCAGACCAATTCCCAGAGGAAGCAAAGAAACCATTACAGAGACT
ACAAGGGGGAAGGGAAGGAGAGATGAATTAGCTTCCCCTGTAAACCTTAGAACCCAGCTGTTGCCA
GGGCAACGGGGCAATACCTGTCTCTTCAGAGGAGATGAAGTTGCCAGGGTAACTACATCCTGTCTTT
CTCAAGGACCATCCCAGAATGTGGCACCCACTAGCCGTTACCATAGCAACTGCCTCTTTGCCCCACT
TAATCCCATCCCGTCTGTTAAAAGGGCCCTATAGTTGGAGGTGGGGGAGGTAGGAAGAGCGATGAT
CACTTGTGGACTAAGTTTGTTCGCATCCCCTTCTCCAACCCCCTCAGTACATCACCCTGGGGGAACA
GGGTCCACTTGCTCCTGGGCCCACACAGTCCTGCAGTATTGTGTATATAAGGCCAGGGCAAAGAGG
AGCAGGTTTTAAAGTGAAAGGCAGGCAGGTGTTGGGGAGGCAGTTACCGGGGCAACGGGAACAGG
GCGTTTCGGAGGTGGTTGCCATGGGGACCTGGATGCTGACGAAGGCTCGCGAGGCTGTGAGCAGC
CACAGTGCCCTGCTCAGAAGCCCCAAGCTCGTCAGTCAAGCCGGTTCTCCGTTTGCACTCAGGAGC
ACGGGCAGGCGAGTGGCCCCTAGTTCTGGGGGCAGCGAATTCCAATTGGCGCGCCTAGCCCGGGC
TAGGTCGACACGTTTCCAGAACGTCTGTAGCTTTTCTCCTCCTTCCCTCCATTTTCCTCTTGGTCTTA
CCTTTGGCCTAGTGGTTGGTGTAGTGATAATGTAGCGAGATTTTCTG [symbol] [symbol]
GGTTGCGAGGTATGAGTAAA [symbol] TGGAACGTCACGCAGCTTTCTACA
GCATGACAAGCTGCTGAGGCTTAAATCAGGATTTTCCTGTCTCTTTCTACAAAATCAAAATGAAAAAA
GAGGGCTTTTTAGGCATCTCCGAGATTATGTGACCGGTCTCGAGGGATCCGATCTTTTTGCCTCTGC
CAAAAATTATGGGGACATCATGAAGCCCCTTGAGCATCTGACTTCTGGCTAATAAAGGAAATTTATTT
TCATTGCAATAGTGTGTTGGAATTTTTTGTGTCTCTCACTCGGCGGCCGCCCGAGTTTAATTGGTTTA
TAGAACTCTTCAAGCTAGCGAAGCAATTCGTTGATCTGAATTTCGACCACCCATAATACCCATTACCC
TGGTAGATAAGTAGCATGGCGGGTTAATCATTAACTACAaggaacccctagtgatggagttggccactccctctctg
cgcgctcgctcgctcactgaggccgggcgaccaaaggtcgcccgacgcccgggctttgcccgggcggcctcagtgagcgagcga
gcgcgcag

Key:
Bold = 5’ ITR sequence; single underline = promoter sequence; italics = 5’ flanking sequence-guide sequence-microRNA loop sequence-passenger sequence-3’ flanking sequence; italics + wave underline: DNA encoding the passenger sequence complementary to a guide sequence (G9 and MW-in that order); italics + bold + single underline: DNA encoding the guide sequence (G9 and MW-in that order); double underline: polyA sequence; bold + lowercase letters: 3’ ITR sequence (SEQ ID NO: 789).

Another exemplary dual-miRNA, anti-Grik2 construct of the disclosure may include an AAV (e.g., AAV9) construct containing an hSyn promoter (SEQ ID NO: 790) operably linked to a GI (SEQ ID NO: 77) ASO sequence incorporated into an E-miR-30-3 scaffold followed by a CaMKII promoter (SEQ ID NO: 802) operably linked to a G9 (SEQ ID NO: 68) ASO sequence incorporated into an E-miR-124-3 scaffold (DMTPV3). Such a construct may have the nucleic acid sequence of SEQ ID NO: 787 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 787 (see below).

(SEQ ID NO: 787)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGATCCGGT
CGGGCCCGCGGTACCGTCGAGAAGCTTGATGTGGGCGGAGCTTCGAAGGGGCGGGCGCCCGTGG
GGCGGGTCCTGAGTGGGGGCGGGACCGGGGCCGGCACCTGGGTGAGGTTCTGCAGAGGGCCCTG
CGTATGAGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCAGAGAGGG
GGAGGGGAAACAGGATGCGGCGAGGCGCGTGCGCACTGCCAGCTTCAGCACCGCGGACAGTGCC
TTCGCCCCCGCCTGGCGGCGCGCGCCACCGCCGCCTCAGCACTGAAGGCGCGCTGACGTCACTC
GCCGGTCCCCCGCAAACTCCCCTTCCCGGCCACCTTGGTCGCGTCCGCGCCGCCGCCGGCCCAGC
CGGACCGCACCACGCGAGGCGCGAGATAGGGGGGCACGGGCGCGACCATCTGCGCTGCGGCGCC
GGCGACTCAGCGCTGCCTCAGTCTGCGGTGGGCAGCGGAGGAGTCGTGTCGTGCCTGAGAGCGCA
GGGCGCGCCTAGCCCGGGCTAGGTCGACTCGACTAGGGATAACAGGGTAATTGTTTGAATGAGGCT
TCAGTACTTTACAGAATCGTTGCCTGCACATCTTGGAAACACTTGCTGGGATTACTTCTTCAGGTTAA
CCCAACAGAAGGCTAAAGAAGGTATATTGCTGTTGACAGTGAGCGAC [symbol] [symbol]
CTGTGAAGCCACAGATGGG [symbol] GCTGCCTACTGCCTCGGACTTCAA
GGGGCTACTTTAGGAGCAATTATCTTGTTTACTAAAACTGAATACCTTGCTATCTCTTTGATACATTTT
TACAAAGCTGAATTAAAATGGTATAAATTATCACGGGATCCCTGTGCCTTCTAGTTGCCAGCCATCTG
TTGTTTGCCCCTCCCCCGTGCCTTCCTTGACCCTGGAAGGTGCCACTCCCACTGTCCTTTCCTAATA
AAATGAGGAAATTGCATCGCATTGTCTGAGTAGGTGTCATTCTATTCTGGGGGGTGGGGTGGGGCA
GGACAGCAAGGGGGAGGATTGGGAAGACAATAGCAGGCATGCTGGGGAGGCGGCCGCCCGAGTT
TAATTGGTTTATAGAACTCTTCAAGCTAGCACTAGTGAAGCAATTCGTTGCATTATGGCCTTAGGTCA
CTTCATCTCCATGGGGTTCTTCTTCTGATTTTCTAGAAAATGAGATGGGGGTGCAGAGAGCTTCCTCA
GTGACCTGCCCAGGGTCACATCAGAAATGTCAGAGCTAGAACTTGAACTCAGATTACTAATCTTAAAT
TCCATGCCTTGGGGGCATGCAAGTACGATATACAGAAGGAGTGAACTCATTAGGGCAGATGACCAAT
GAGTTTAGGAAAGAAGAGTCCAGGGCAGGGTACATCTACACCACCCGCCCAGCCCTGGGTGAGTCC
AGCCACGTTCACCTCATTATAGTTGCCTCTCTCCAGTCCTACCTTGACGGGAAGCACAAGCAGAAAC
TGGGACAGGAGCCCCAGGAGACCAAATCTTCATGGTCCCTCTGGGAGGATGGGTGGGGAGAGCTG
TGGCAGAGGCCTCAGGAGGGGCCCTGCTGCTCAGTGGTGACAGATAGGGGTGAGAAAGCAGACAG
AGTCATTCCGTCAGCATTCTGGGTCTGTTTGGTACTTCTTCTCACGCTAAGGTGGCGGTGTGATATG
CACAATGGCTAAAAAGCAGGGAGAGCTGGAAAGAAACAAGGACAGAGACAGAGGCCAAGTCAACCA
GACCAATTCCCAGAGGAAGCAAAGAAACCATTACAGAGACTACAAGGGGGAAGGGAAGGAGAGATG
AATTAGCTTCCCCTGTAAACCTTAGAACCCAGCTGTTGCCAGGGCAACGGGGCAATACCTGTCTCTT
CAGAGGAGATGAAGTTGCCAGGGTAACTACATCCTGTCTTTCTCAAGGACCATCCCAGAATGTGGCA
CCCACTAGCCGTTACCATAGCAACTGCCTCTTTGCCCCACTTAATCCCATCCCGTCTGTTAAAAGGG
CCCTATAGTTGGAGGTGGGGGAGGTAGGAAGAGCGATGATCACTTGTGGACTAAGTTTGTTCGCAT
CCCCTTCTCCAACCCCCTCAGTACATCACCCTGGGGGAACAGGGTCCACTTGCTCCTGGGCCCACA
CAGTCCTGCAGTATTGTGTATATAAGGCCAGGGCAAAGAGGAGCAGGTTTTAAAGTGAAAGGCAGG
CAGGTGTTGGGGAGGCAGTTACCGGGGCAACGGGAACAGGGCGTTTCGGAGGTGGTTGCCATGGG
GACCTGGATGCTGACGAAGGCTCGCGAGGCTGTGAGCAGCCACAGTGCCCTGCTCAGAAGCCCCA
AGCTCGTCAGTCAAGCCGGTTCTCCGTTTGCACTCAGGAGCACGGGCAGGCGAGTGGCCCCTAGTT
CTGGGGGCAGCGAATTCCAATTGGCGCGCCTAGCCCGGGCTAGGTCGACTCTGCCGCGGAAAGGG
GAGAAGTGTGGGCTCCTCCGAGTCGGGGGCGGACTGGGACAGCACAGTCGGCTGAGCGCAGCGC
CCCCGCCCTGCCCGCCACGCGGCGAAGACGCCTGAGCGTTCGCGCCCCTCGGGCGAGGACCCCA
CGCAAGCCCGAGCCGGTCCCGACCCTGGCCCCGACGCTCGCCGCCCGCCCCAGCCCTGAGGGCC
CCTCTA [symbol] TTAATGTCTATACAAT [symbol] GA
GAGGCGCCTCCGCCGCTCCTTTCTCATGGAAATGGCCCGCGAGCCCGTCCGGCCCAGCGCCCCTC
CCGCGGGAGGAAGGCGAGCCCGGCCCCCGGCGGCCATTCGCGCCGCGGACAAATCCGGCGAACA
ATGCGCCCGCCCAGAGTGCGGCCCAGCTGCCGGGCCGGGGATCTGGCCGCGGGACACAAAGGGG
CCCGCACGCCTCTGGCGTACCGGTCTCGAGGGATCCGATCTTTTTCCCTCTGCCAAAAATTATGGG
GAGATGATGAAGGGGGTTGAGGATGTGAGTTGTGGGTAATAAAGGAAATTTATTTTGATTGGAATAGT
GTGTTGGAATTTTTTGTGTCTCTCACTCGGCGGCCGCCCGAGTTTAATTGGTTTATAGAACTCTTCAA
GCTAGCGAAGCAATTCGTTGATCTGAATTTCGACCACCCATAATACCCATTACCCTGGTAGATAAGTA
GCATGGCGGGTTAATCATTAACTACAaggaacccctagtgatggagttggccactccctctctgcgcgctcgctcgctcac
tgaggccgggcgaccaaaggtcgcccgacgcccgggctttgcccgggcggcctcagtgagcgagcgagcgcgcag

Key:
Bold = 5’ ITR sequence; single underline = promoter sequence; italics = 5’ flanking sequence-guide sequence-microRNA loop sequence-passenger sequence-3’ flanking sequence; italics + wave underline: DNA encoding the passenger sequence complementary to a guide sequence (GI and G9-in that order); italics + bold + single underline: DNA encoding the guide sequence (GI and G9-in that order); double underline: polyA sequence; bold + lowercase letters: 3’ ITR sequence (SEQ ID NO: 789).

Another exemplary dual-miRNA, anti-Grik2 construct of the disclosure may include an AAV (e.g., AAV9) construct containing an hSyn promoter (SEQ ID NO: 790) operably linked to a GI (SEQ ID NO: 77) ASO sequence incorporated into an E-miR-30-3 scaffold followed by a CaMKII promoter (SEQ ID NO: 802) operably linked to an MW (SEQ ID NO: 80) ASO sequence incorporated into an E-miR-124-3 scaffold (DMTPV4). Such a construct may have the nucleic acid sequence of SEQ ID NO: 788 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 788 (see below).

(SEQ ID NO: 788)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGATCCGGT
CGGGCCCGCGGTACCGTCGAGAAGCTTGATGTGGGCGGAGCTTCGAAGGGGCGGGCGCCCGTGG
GGCGGGTCCTGAGTGGGGGCGGGACCGGGGCCGGCACCTGGGTGAGGTTCTGCAGAGGGCCCTG
CGTATGAGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCAGAGAGGG
GGAGGGGAAACAGGATGCGGCGAGGCGCGTGCGCACTGCCAGCTTCAGCACCGCGGACAGTGCC
TTCGCCCCCGCCTGGCGGCGCGCGCCACCGCCGCCTCAGCACTGAAGGCGCGCTGACGTCACTC
GCCGGTCCCCCGCAAACTCCCCTTCCCGGCCACCTTGGTCGCGTCCGCGCCGCCGCCGGCCCAGC
CGGACCGCACCACGCGAGGCGCGAGATAGGGGGGCACGGGCGCGACCATCTGCGCTGCGGCGCC
GGCGACTCAGCGCTGCCTCAGTCTGCGGTGGGCAGCGGAGGAGTCGTGTCGTGCCTGAGAGCGCA
GGGCGCGCCTAGCCCGGGCTAGGTCGACTCGACTAGGGATAACAGGGTAATTGTTTGAATGAGGCT
TCAGTACTTTACAGAATCGTTGCCTGCACATCTTGGAAACACTTGCTGGGATTACTTCTTCAGGTTAA
CCCAACAGAAGGCTAAAGAAGGTATATTGCTGTTGACAGTGAGCGAC [symbol] [symbol]
CTGTGAAGCCACAGATGGG [symbol] GCTGCCTACTGCCTCGGACTTCAA
GGGGCTACTTTAGGAGCAATTATCTTGTTTACTAAAACTGAATACCTTGCTATCTCTTTGATACATTTT
TACAAAGCTGAATTAAAATGGTATAAATTATCACGGGATCCCTGTGCCTTCTAGTTGCCAGCCATCTG
TTGTTTGCCCCTCCCCCGTGCCTTCCTTGACCCTGGAAGGTGCCACTCCCACTGTCCTTTCCTAATA
AAATGAGGAAATTGCATCGCATTGTCTGAGTAGGTGTCATTCTATTCTGGGGGGTGGGGTGGGGCA
GGACAGCAAGGGGGAGGATTGGGAAGACAATAGCAGGCATGCTGGGGAGGCGGCCGCCCGAGTT
TAATTGGTTTATAGAACTCTTCAAGCTAGCACTAGTGAAGCAATTCGTTGCATTATGGCCTTAGGTCA
CTTCATCTCCATGGGGTTCTTCTTCTGATTTTCTAGAAAATGAGATGGGGGTGCAGAGAGCTTCCTCA
GTGACCTGCCCAGGGTCACATCAGAAATGTCAGAGCTAGAACTTGAACTCAGATTACTAATCTTAAAT
TCCATGCCTTGGGGGCATGCAAGTACGATATACAGAAGGAGTGAACTCATTAGGGCAGATGACCAAT
GAGTTTAGGAAAGAAGAGTCCAGGGCAGGGTACATCTACACCACCCGCCCAGCCCTGGGTGAGTCC
AGCCACGTTCACCTCATTATAGTTGCCTCTCTCCAGTCCTACCTTGACGGGAAGCACAAGCAGAAAC
TGGGACAGGAGCCCCAGGAGACCAAATCTTCATGGTCCCTCTGGGAGGATGGGTGGGGAGAGCTG
TGGCAGAGGCCTCAGGAGGGGCCCTGCTGCTCAGTGGTGACAGATAGGGGTGAGAAAGCAGACAG
AGTCATTCCGTCAGCATTCTGGGTCTGTTTGGTACTTCTTCTCACGCTAAGGTGGCGGTGTGATATG
CACAATGGCTAAAAAGCAGGGAGAGCTGGAAAGAAACAAGGACAGAGACAGAGGCCAAGTCAACCA
GACCAATTCCCAGAGGAAGCAAAGAAACCATTACAGAGACTACAAGGGGGAAGGGAAGGAGAGATG
AATTAGCTTCCCCTGTAAACCTTAGAACCCAGCTGTTGCCAGGGCAACGGGGCAATACCTGTCTCTT
CAGAGGAGATGAAGTTGCCAGGGTAACTACATCCTGTCTTTCTCAAGGACCATCCCAGAATGTGGCA
CCCACTAGCCGTTACCATAGCAACTGCCTCTTTGCCCCACTTAATCCCATCCCGTCTGTTAAAAGGG
CCCTATAGTTGGAGGTGGGGGAGGTAGGAAGAGCGATGATCACTTGTGGACTAAGTTTGTTCGCAT
CCCCTTCTCCAACCCCCTCAGTACATCACCCTGGGGGAACAGGGTCCACTTGCTCCTGGGCCCACA
CAGTCCTGCAGTATTGTGTATATAAGGCCAGGGCAAAGAGGAGCAGGTTTTAAAGTGAAAGGCAGG
CAGGTGTTGGGGAGGCAGTTACCGGGGCAACGGGAACAGGGCGTTTCGGAGGTGGTTGCCATGGG
GACCTGGATGCTGACGAAGGCTCGCGAGGCTGTGAGCAGCCACAGTGCCCTGCTCAGAAGCCCCA
AGCTCGTCAGTCAAGCCGGTTCTCCGTTTGCACTCAGGAGCACGGGCAGGCGAGTGGCCCCTAGTT
CTGGGGGCAGCGAATTCCAATTGGCGCGCCTAGCCCGGGCTAGGTCGACTCTGCCGCGGAAAGGG
GAGAAGTGTGGGCTCCTCCGAGTCGGGGGCGGACTGGGACAGCACAGTCGGCTGAGCGCAGCGC
CCCCGCCCTGCCCGCCACGCGGCGAAGACGCCTGAGCGTTCGCGCCCCTCGGGCGAGGACCCCA
CGCAAGCCCGAGCCGGTCCCGACCCTGGCCCCGACGCTCGCCGCCCGCCCCAGCCCTGAGGGCC
CCTCGA [symbol] TTAATGTCTATACAAT [symbol] G
AGAGGCGCCTCCGCCGCTCCTTTCTCATGGAAATGGCCCGCGAGCCCGTCCGGCCCAGCGCCCCT
CCCGCGGGAGGAAGGCGAGCCCGGCCCCCGGCGGCCATTCGCGCCGCGGACAAATCCGGCGAAC
AATGCGCCCGCCCAGAGTGCGGCCCAGCTGCCGGGCCGGGGATCTGGCCGCGGGACACAAAGGG
GCCCGCACGCCTCTGGCGTACCGGTCTCGAGGGATCCGATCTTTTTCCCTCTGCCAAAAATTATGG
GGACATCATGAAGCCCCTTGAGCATCTGACTTCTGGCTAATAAAGGAAATTTATTTTCATTGCAATAG
TGTGTTGGAATTTTTTGTGTCTCTCACTCGGCGGCCGCCCGAGTTTAATTGGTTTATAGAACTCTTCA
AGCTAGCGAAGCAATTCGTTGATCTGAATTTCGACCACCCATAATACCCATTACCCTGGTAGATAAGT
AGCATGGCGGGTTAATCATTAACTACAaggaacccctagtgatggagttggccactccctctctgcgcgctcgctcgctca
ctgaggccgggcgaccaaaggtcgcccgacgcccgggctttgcccgggcggcctcagtgagcgagcgagcgcgcag

Key:
Bold = 5’ ITR sequence; single underline = promoter sequence; italics = 5’ flanking sequence-guide sequence-microRNA loop sequence-passenger sequence-3’ flanking sequence; italics + wave underline: DNA encoding the passenger sequence complementary to a guide sequence (GI and MW-in that order); italics + bold + single underline: DNA encoding the guide sequence (GI and MW-in that order); double underline: polyA sequence; bold + lowercase letters: 3’ ITR sequence (SEQ ID NO: 789).

Another exemplary dual-miRNA, anti-Grik2 construct of the disclosure may include an AAV (e.g., AAV9) construct containing an hSyn promoter (SEQ ID NO: 790) operably linked to a GI (SEQ ID NO: 77) ASO sequence incorporated into an E-miR-30-3 scaffold followed by a CaMKII promoter (SEQ ID NO: 802) operably linked to an MW (SEQ ID NO: 80) ASO sequence incorporated into an E-miR-218-1 scaffold (DMTPV8). Such a construct may have the nucleic acid sequence of SEQ ID NO: 813 or can be a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 813 (see below). Such an expression cassette may be incorporated into a vector having the nucleic acid sequence of SEQ ID NO: 814 or a variant thereof having at least 85% (at least 86%, 87%, 88%, 89%, 90%, 91%, 92%, 93%, 94%, 95%, 96%, 97%, 98%, 99%, or more) sequence identity to the nucleic acid sequence of SEQ ID NO: 814.

(SEQ ID NO: 813)
CTGCAGAGGGCCCTGCGTATGAGTGCAAGTGGGTTTTAGGACCAGGATGAGGCGGGGTGGGGGTG
CCTACCTGACGACCGACCCCGACCCACTGGACAAGCACCCAACCCCCATTCCCCAAATTGCGCATC
CCCTATCAGAGAGGGGGAGGGGAAACAGGATGCGGCGAGGCGCGTGCGCACTGCCAGCTTCAGC
ACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCA
TCTGCGCTGCGGCGCCGGCGACTCAGCGCTGCCTCAGTCTGCGGTGGGCAGCGGAGGAGTCGTG
TCGTGCCTGAGAGCGCAGGGCGCGCCTAGCCCGGGCTAGGTCGACTCGACTAGGGATAACAGGG
TAATTGTTTGAATGAGGCTTCAGTACTTTACAGAATCGTTGCCTGCACATCTTGGAAACACTTGCTG
GGATTACTTCTTCAGGTTAACCCAACAGAAGGCTAAAGAAGGTATATTGCTGTTGACAGTGAGCGA
CGTCTCGATATGGAGAACCCATGCTGTGAAGCCACAGATGGGCATGGGTTTTATATCGAGACGCT
GCCTACTGCCTCGGACTTCAAGGGGCTACTTTAGGAGCAATTATCTTGTTTACTAAAACTGAATAC
CTTGCTATCTCTTTGATACATTTTTACAAAGCTGAATTAAAATGGTATAAATTATCACGGGATCCCT
GTGCCTTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGAGGCGGCCGCCCGAGTTTAATTGGTTTATAGAACTCTTCAAGCTAGCACTAGTGAAGCAAT
TCGTTGCATTATGGCCTTAGGTCACTTCATCTCCATGGGGTTCTTCTTCTGATTTTCTAGAAAATGAG
ATGGGGGTGCAGAGAGCTTCCTCAGTGACCTGCCCAGGGTCACATCAGAAATGTCAGAGCTAGAAC
TTGAACTCAGATTACTAATCTTAAATTCCATGCCTTGGGGGCATGCAAGTACGATATACAGAAGGAGT
GAACTCATTAGGGCAGATGACCAATGAGTTTAGGAAAGAAGAGTCCAGGGCAGGGTACATCTACAC
CACCCGCCCAGCCCTGGGTGAGTCCAGCCACGTTCACCTCATTATAGTTGCCTCTCTCCAGTCCTAC
CTTGACGGGAAGCACAAGCAGAAACTGGGACAGGAGCCCCAGGAGACCAAATCTTCATGGTCCCTC
TGGGAGGATGGGTGGGGAGAGCTGTGGCAGAGGCCTCAGGAGGGGCCCTGCTGCTCAGTGGTGA
CAGATAGGGGTGAGAAAGCAGACAGAGTCATTCCGTCAGCATTCTGGGTCTGTTTGGTACTTCTTCT
CACGCTAAGGTGGCGGTGTGATATGCACAATGGCTAAAAAGCAGGGAGAGCTGGAAAGAAACAAGG
ACAGAGACAGAGGCCAAGTCAACCAGACCAATTCCCAGAGGAAGCAAAGAAACCATTACAGAGACT
ACAAGGGGGAAGGGAAGGAGAGATGAATTAGCTTCCCCTGTAAACCTTAGAACCCAGCTGTTGCCA
GGGCAACGGGGCAATACCTGTCTCTTCAGAGGAGATGAAGTTGCCAGGGTAACTACATCCTGTCTTT
CTCAAGGACCATCCCAGAATGTGGCACCCACTAGCCGTTACCATAGCAACTGCCTCTTTGCCCCACT
TAATCCCATCCCGTCTGTTAAAAGGGCCCTATAGTTGGAGGTGGGGGAGGTAGGAAGAGCGATGAT
CACTTGTGGACTAAGTTTGTTCGCATCCCCTTCTCCAACCCCCTCAGTACATCACCCTGGGGGAACA
GGGTCCACTTGCTCCTGGGCCCACACAGTCCTGCAGTATTGTGTATATAAGGCCAGGGCAAAGAGG
AGCAGGTTTTAAAGTGAAAGGCAGGCAGGTGTTGGGGAGGCAGTTACCGGGGCAACGGGAACAGG
GCGTTTCGGAGGTGGTTGCCATGGGGACCTGGATGCTGACGAAGGCTCGCGAGGCTGTGAGCAGC
CACAGTGCCCTGCTCAGAAGCCCCAAGCTCGTCAGTCAAGCCGGTTCTCCGTTTGCACTCAGGAGC
ACGGGCAGGCGAGTGGCCCCTAGTTCTGGGGGCAGCGAATTCCAATTGGCGCGCCTAGCCCGGGC
TAGGTCGACACGTTTCCAGAACGTCTGTAGCTTTTCTCCTCCTTCCCTCCATTTTCCTCTTGGTCTTA
CCTTTGGCCTAGTGGTTGGTGTAGTGATAATGTAGCGAGATTTTCTGCAGAGCATTGCAGATGGAC
TGGGTTGCGAGGTATGAGTAAACAGTCCATACGCAATGCTCCGTGGAACGTCACGCAGCTTTCTA
CAGCATGACAAGCTGCTGAGGCTTAAATCAGGATTTTCCTGTCTCTTTCTACAAAATCAAAATGAA
AAAAGAGGGCTTTTTAGGCATCTCCGAGATTATGTGACCGGTCTCGAGGGATCCGATCTTTTTCCC
TCTGCCAAAAATTATGGGGACATCATGAAGCCCCTTGAGCATCTGACTTCTGGCTAATAAAGGAAATT
TATTTTCATTGCAATAGTGTGTTGGAATTTTTTGTGTCTCTCACTCGGCGGCCGCCCGAGTTTAATTG
GTTTATAGAACTCTTCA

Key:
single underline: promoter sequence; bold: microRNA stem-loop structure containing guide and passenger sequences; double underline: polyA sequence; italics: stutter sequence (SEQ ID NO: 816).

Improvements in the manufacture of AAV vectors containing multiple miRNA sequences

Preparation of AAV vectors using plasmids encoding a single miRNA expression cassette (e.g., expression cassettes disclosed herein) may potentially be hampered by improper AAV genome packaging. First, pri-miRNA sequences are short (<200 bases) and, depending on promoter length, design of a transgene cassette with a single promoter controlling expression of a single miRNA may result in an AAV genome that is significantly shorter than the maximum packaging capacity of AAV (˜4.8 kb). It is therefore possible that a single capsid may be loaded with more than one vector if the anticipated full genome length is <2.4 kb (half the packaging capacity of AAV). This can be mediated by polymerase read-through without proper endonuclease nicking that allows for the production of AAV genome dimers (or trimers) that can then be packaged into the AAV capsid if they are of appropriate length. This subsequently introduces significant heterogeneity into the population of AAV vector particles, which renders manufacturing and characterization of a drug product significantly more difficult.

Second, shRNA- and miRNA-based transgenes inherently have significant secondary structure due to the inclusion of the miRNA hairpin. It has been shown that these internal secondary structures within an AAV genome can function as a “false” ITR during AAV genome replication and packaging, resulting in truncation events and a heterogeneous population of AAV vector particles containing a mixture of full and partial vectors.

We have discovered that padding of an AAV genome having sizes below the AAV packaging capacity with additional sequences (e.g., additional pre-miRNA stem-loop sequences, a second promoter sequence, stuffer sequences (e.g., SEQ ID NO: 815 and/or SEQ ID NO: 816), using self-complementary AAV vectors, etc.) substantially improves AAV packaging by avoiding incorporation of copies of partial (i.e., truncated) AAV genomes. Therefore, the constructs described herein avoid improper packaging of AAV genomes by incorporating the aforementioned sequences into an AAV expression cassette to increase vector size to a value closer to the maximal AAV packaging capacity.

For example, there may be cases where a construct expressing one miRNA under control of a single promoter or two miRNAs from two separate promoters (dual construct approach) may not perform in vivo as predicted by in vitro/ex vivo/in silico evaluation. In these cases, the following strategies can be implemented to establish a genomic length that produces a homogeneous, full-length, singly packaged population of AAV vector particles.

First, if expression of a single miRNA “guide” is desired, a stuffer sequence (e.g., SEQ ID NO: 815 and/or SEQ ID NO: 816) may be added to increase the total length of the AAV vector without disrupting the promoter.miR cassette itself. This stuffer may be added downstream of the transgene cassette (3′ of polyA sequence)(see, e.g., FIG. 6F and FIG. 6G). The length and content of this stuffer may be changed while retaining its ability to improve AAV packaging homogeneity. Additionally, this stuffer may be placed upstream (5′) of the promoter. Stuffer sequences may be utilized in the context of an scAAV vector or a single-stranded (ss)AAV vector. In some embodiments, the vector of the disclosure includes one or more stuffer sequences (e.g., 1, 2, or more stuffer sequences). In some embodiments, the one or more stuffer sequences have at least 85% (e.g., at least 86%, 90%, 95%, 96%, 97%, 98%, 99%, or more) sequence identity to the nucleic acid sequence of SEQ ID NO: 815. In some embodiments, the one or more stuffer sequences have at least 90% (e.g., at least 91%, 95%, 96%, 97%, 98%, 99%, or more) sequence identity to the nucleic acid sequence of SEQ ID NO: 815. In some embodiments, the one or more stuffer sequences have at least 95% (e.g., at least 96%, 97%, 98%, 99%, or more) sequence identity to the nucleic acid sequence of SEQ ID NO: 815. In some embodiments, the one or more stuffer sequences have at least 99% sequence identity to the nucleic acid sequence of SEQ ID NO: 815. In some embodiments, the one or more stuffer sequences have the nucleic acid sequence of SEQ ID NO: 815. In some embodiments, the one or more stuffer sequences have at least 85% (e.g., at least 86%, 90%, 95%, 96%, 97%, 98%, 99%, or more) sequence identity to the nucleic acid sequence of SEQ ID NO: 816. In some embodiments, the one or more stuffer sequences have at least 90% (e.g., at least 91%, 95%, 96%, 97%, 98%, 99%, or more) sequence identity to the nucleic acid sequence of SEQ ID NO: 816. In some embodiments, the one or more stuffer sequences have at least 95% (e.g., at least 96%, 97%, 98%, 99%, or more) sequence identity to the nucleic acid sequence of SEQ ID NO: 816.

In some embodiments, the one or more stuffer sequences have at least 99% sequence identity to the nucleic acid sequence of SEQ ID NO: 816. In some embodiments, the one or more stuffer sequences have the nucleic acid sequence of SEQ ID NO: 816.

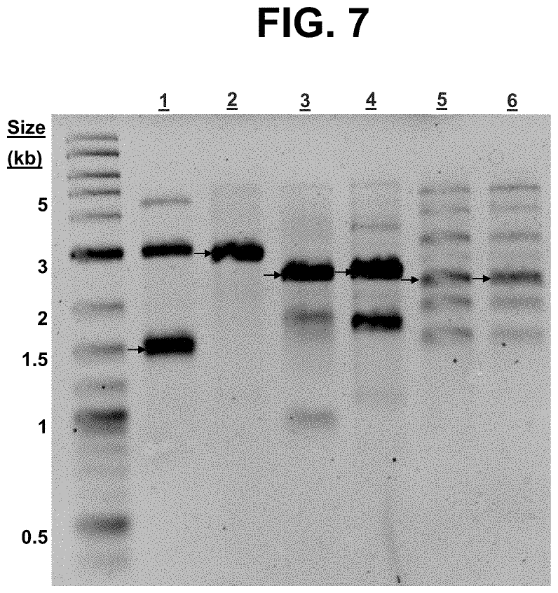

Second, if more than one miRNA is to be expressed but a single promoter strategy is selected, the vector can be prepared by concatemerization of multiple miRNA cassettes. While concatemerization of multiple miRNA cassettes (e.g., up to miRNA cassettes) using the same scaffold can result in recombination between homologous sequences within the vector (FIG. 7), concatemerization of miRNA cassettes using different scaffolds with non-homologous flanking and loop sequences can ameliorate this packaging concern (FIG. 8F). If the inclusion of additional miRNA cassettes does not result in a vector of appropriate length, a stuffer sequence (e.g., SEQ ID NO: 815 and/or SEQ ID NO: 816) may be incorporated as described above to increase length to improve packaging efficiency.

### Pharmaceutical Compositions

The oligonucleotides described herein or nucleic acid vectors encoding the same may be formulated into pharmaceutical compositions for administration to a mammalian (e.g., a human) subject in a biologically compatible form suitable for administration in vivo.

The compositions disclosed herein may be formulated in any suitable vehicle for delivery to a subject (e.g., a human). For instance, they may be formulated in a pharmaceutically acceptable suspension, dispersion, solution, or emulsion. Suitable mediums include saline and liposomal preparations. More specifically, pharmaceutically acceptable carriers may include sterile aqueous of non-aqueous solutions, suspensions, and emulsions. Recombinant human album (rAlbumin Human NF RECOMBUMIN® Prime) may also be used as a stabilizer with an AAV vector (Albumedix, Nottingham UK). Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles suitable for intravenous administration include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

The compositions described herein may be used in the form of the free base, in the form of salts, solvates, and as prodrugs. All forms are within the methods described herein. In accordance with the methods of the disclosure, the described compounds or salts, solvates, or prodrugs thereof may be administered to a patient in a variety of forms depending on the selected route of administration.

Accordingly, the compositions described herein may be formulated for administration, for example, by oral, parenteral, intrathecal, intracerebroventricular, intraparenchymal, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration. Parenteral administration may be by continuous infusion over a selected period of time.

Solutions of an agent described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Local, regional, or systemic administration also may be appropriate. A composition described herein may advantageously be contacted by administering an injection or multiple injections to the target site, spaced for example, at approximately, 1 cm intervals.

The compositions described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

Accordingly, the present disclosure relates to a pharmaceutical composition containing an ASO agent disclosed herein (e.g., siRNA, shRNA, miRNA, or shmiRNA, or shmiRNA). In particular, the present disclosure relates to a composition including a vector including an ASO agent of the disclosure.

In a particular example, the disclosure provides a pharmaceutical composition containing a vector (e.g., lentiviral or AAV vector) including an ASO of the disclosure operably linked to a promoter, as is disclosed herein. The pharmaceutical composition may include an AAV vector including (a) a viral capsid; and (b) an artificial polynucleotide including an expression cassette flanked by AAV ITRs, wherein the expression cassette includes a polynucleotide encoding an oligonucleotide that binds to and inhibits the expression of a Grik2 mRNA, operably linked to one or more regulatory sequences that control expression of the polynucleotide in CNS cells.

The ASO agents disclosed herein may be combined with pharmaceutically acceptable excipients, and, optionally, sustained-release matrices, such as, e.g., biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions disclosed herein may be formulated for intracerebral (e.g., intraparenchymal or intracerebroventricular), intramuscular, intravenous, transdermal, local, oral, sublingual, subcutaneous, or rectal administration. The active component of the composition (e.g., an ASO agent targeting Grik2), alone or in combination with another therapeutic agent, can be administered in a unit administration form as a mixture with conventional pharmaceutical supports to subjects in need thereof. Suitable unit administration forms include oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal, intracerebral, stereotactic, and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions, formulations including sesame oil, peanut oil, or aqueous propylene glycol, and sterile powders for the extemporaneous preparation of sterile injectable solutions or dispersions. In all cases, the form must be sterile and must be fluid to the extent that easy syringability is facilitated. It must be stable under the conditions of manufacture and storage and must be preserved against the contaminating action of microorganisms, such as bacteria and fungi. Solutions including compounds of the disclosure as free base or pharmacologically acceptable salts can be prepared in water suitably mixed with a surfactant, such as hydroxypropyl cellulose. Dispersions can also be prepared in glycerol, liquid polyethylene glycols, and mixtures thereof and in oils. Under ordinary conditions of storage and use, these preparations may contain a preservative to prevent the growth of microorganisms. The oligonucleotide agents disclosed herein can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids like acetic, oxalic, tartaric, mandelic, and the like. Salts formed with the free carboxyl groups can also be derived from inorganic bases such as, e.g., sodium, potassium, ammonium, calcium, or ferric hydroxides, and such organic bases as isopropylamine, trimethylamine, histidine, procaine, and the like. The carrier can also be a solvent or dispersion medium containing, e.g., water, ethanol, polyol (for example, glycerol, propylene glycol, and liquid polyethylene glycol, and the like), suitable mixtures thereof, and vegetables oils. The proper fluidity can be maintained by the use of a coating (e.g., lecithin), by the maintenance of the required particle size in the case of dispersion, and by the use of surfactants. The prevention of the action of microorganisms can be brought about by various antibacterial and antifungal agents such as, e.g., parabens, chlorobutanol, phenol, sorbic acid, thimerosal, and the like. In some cases, it will be pharmaceutical compositions of the disclosure may include isotonic agents such as, e.g., sugars or sodium chloride. Prolonged absorption of the injectable compositions can be brought about by the use of agents delaying absorption such as, e.g., e.g., aluminum monostearate and gelatin. Sterile injectable solutions are prepared by incorporating the active agents in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required additional ingredients from those disclosed herein. In the case of sterile powders for the preparation of sterile injectable solutions, the preferred methods of preparation may be vacuum-drying and freeze-drying techniques which yield a powder of the active ingredient plus any additional desired ingredient from a previously sterile-filtered solution thereof. Upon formulation, solutions will be administered in a manner compatible with the dosage requirement and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. Sterile aqueous media which can be employed are well-known in the art. For example, one dosage could be dissolved in 1 mL of isotonic NaCl solution and added to 1000 mL of hypodermoclysis fluid or injected at the proposed site of infusion. Some variation in dosage will necessarily occur depending on the condition of the subject being treated. The practitioner responsible for administration can, in any event, using appropriate patient information and art-recognized methods, determine the appropriate dose for the individual subject.

### Methods of Diagnosis

A subject (e.g., a human subject) may, e.g., using methods well-known in the art, be diagnosed as having epilepsy (e.g., TLE), and, thus, identified as in need of treatment using the compositions and methods disclosed herein. For example, diagnosis of an epilepsy in a subject may be guided by neurophysiological testing to identify the epileptogenic focus and the severity of epileptiform activity in the brain of the subject. Exemplary neurophysiological testing methods well-known in the art include electroencephalography (EEG), magnetoencephalography (MEG), functional MRI (fMRI), single-photon emission computed tomography (SPECT), and positron emission tomography (PET). EEG and MEG provide a continuous measure of cortical function with high temporal resolution and facilitate the detection of interictal (period between seizures) epileptiform discharges, which may be indicative of a positive diagnosis of an epileptic condition in the subject. Comparison of brain activity in the subject relative to a norm appropriate for the subject's age, medical history, and lifestyle (e.g., a reference population, such as, e.g., non-epileptic patient population) may be done to determine the diagnosis with respect to an epilepsy in the subject.

The subject may be diagnosed as having any one of a number of epileptic conditions including, but not limited to a TLE (e.g., mTLE or ITLE), benign Rolandic epilepsy, a frontal lobe epilepsy, infantile spasms, a juvenile myoclonic epilepsy, a juvenile absence epilepsy, a childhood absence epilepsy (pyknolepsy), a hot water epilepsy, Lennox-Gastaut syndrome, Landau-Kleffner syndrome, Dravet syndrome, a progressive myoclonus epilepsy, a reflex epilepsy, Rasmussen's syndrome, a limbic epilepsy, status epilepticus, an abdominal epilepsy, massive bilateral myoclonus, a catamenial epilepsy, Jacksonian seizure disorder, Lafora disease, and a photosensitive epilepsy. In cases where the epileptic condition is TLE, the TLE may be characterized by characterized by focal or generalized seizures.

The type of epilepsy that a patient can be diagnosed on the basis of localizing the epileptogenic focus to a particular brain region (e.g., mesial temporal lobe, lateral frontal lobe, frontal lobe, etc.) using the disclosed methods. Electrophysiological signature of epileptic brain activity may also be used to identify the particular type or subtype of epilepsy in a subject. For example, the presence of fast (250-600 Hz) sharp wave ripples (SPW-Rs) in cortical regions (e.g., hippocampus or cerebral cortex) may be indicative of a positive diagnosis of a TLE in the subject. In another example, Lennox-Gastaut syndrome is often characterized by the presence of fast-run electrographic oscillations (10-Hz) recorded across the neocortex and thalamus. Furthermore, video monitoring of a subject in an inpatient facility may be indicative of a diagnosis of epilepsy in the patient if the patient appears to overtly exhibit behavioral manifestations of epileptic seizures, such as, e.g., generalized convulsions, temporary absence (decreased levels of consciousness for periods lasting ˜10 seconds), tonus, myoclonus, loss of bowel or bladder control, biting of the tongue, fatigue, headache, difficulty speaking, abnormal behavior (e.g., motionless staring or automatic movements of hands or mouth), psychosis, and/or localized weakness. Self-reported symptoms from the subject being diagnosed for epilepsy may also be indicative of a positive diagnosis. Such self-reported symptoms may include sensations of déjà vu or jamais vu, auras, amnesia, a spontaneous and unprovoked fear and anxiety, nausea, auditory, visual, olfactory, gustatory, or tactile hallucinations, visual distortions (e.g., macropsia or micropsia), dissociation or derealization, synesthesia, dysphoric or euphoric feelings, fear, anger, or ineffable sensations.

### Pharmaceutical Uses

Disclosed herein are methods for the treatment of an epilepsy (e.g., TLE) in a subject diagnosed with or at risk of developing an epileptic condition by administration of the compositions described above (e.g., an ASO agent or a nucleic acid vector encoding the same). Upon administration, the ASO agents of the disclosure are capable of binding to and inhibiting the expression of a Grik2 mRNA. The targeting of Grik2 by an ASO agent disclosed herein may be manifested by a decrease in the levels of Grik2 mRNA expressed by a first cell or group of cells (e.g., neuronal cells; such cells may be present, for example, in the subject or in a sample derived from a subject) in which Grik2 is transcribed and which has or have been treated (e.g., by contacting the cell or cells with an oligonucleotide of the present disclosure, or by administering an oligonucleotide of the disclosure to a subject in which the cells are or were present). In a particular example, the expression of Grik2 is decreased in the first cell or group of cells, as compared to a second cell or group of cells substantially identical to the first cell or group of cells but which has not or have not been so treated (control cell(s) not treated with an oligonucleotide or not treated with an oligonucleotide targeted to the gene of interest). The degree of decrease in the levels of mRNA of a gene of interest (e.g., Grik2) may be expressed in terms of:

$$\frac{( mRNA in control cells ) - ( mRNA in treated cells )}{( mRNA in control cells} \times 100 \%$$

A change in the levels of expression of a gene (e.g., a Grik2 gene) may be assessed in terms of a reduction of a parameter that is functionally linked to the expression of the gene of interest, e.g., protein expression of the gene of interest or signaling downstream of the protein. A change in the levels of expression of the gene of interest may be determined in any cell expressing the gene of interest, either endogenous or heterologous from an expression construct, and by any assay known in the art.

A change in the level of expression of Grik2 may be manifested by a decrease in the level of the GluK2 protein that is expressed by a cell or group of cells (e.g., the level of GluK2 protein expressed in a sample derived from a subject). As is explained above, for the assessment of Grik2 mRNA suppression, the change in the level of GluK2 protein expression in a treated cell or group of cells may similarly be expressed as a percentage of the level of protein in a control cell or group of cells.

A control cell or group of cells that may be used to assess the change in the expression of the Grik2 gene includes a cell or group of cells that has not yet been contacted with an oligonucleotide of the present disclosure. For example, the control cell or group of cells may be derived from an individual subject (e.g., a human or animal subject) prior to treatment of the subject with an oligonucleotide.

The level of Grik2 mRNA expressed by a cell or group of cells may be determined using any method known in the art for assessing mRNA expression. For example, the level of expression Grik2 mRNA in a sample may be determined by detecting a transcribed polynucleotide, or portion thereof, e.g., mRNA. RNA may be extracted from cells using RNA extraction techniques including, for example, using acid phenol/guanidine isothiocyanate extraction (RNAzol B; Biogenesis), RNEASYTM RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating mRNA may be detected using methods the described in PCT Publication WO 2012/177906, the entire contents of which are hereby incorporated herein by reference. The level of expression of the gene of interest may also be determined using a nucleic acid probe. The term “probe,” as used herein, refers to any molecule that is capable of selectively binding to a specific sequence, e.g. to an mRNA. Probes can be synthesized using well-known and conventional methods of the art or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to the mRNA of a gene of interest. The mRNA may be immobilized on a solid surface and contacted with a probe, for example by running the isolated mRNA on an agarose gel and transferring the mRNA from the gel to a membrane, such as nitrocellulose. The probe(s) may also be immobilized on a solid surface and the mRNA is contacted with the probe(s), for example, in an AFFYMETRIX gene chip array. Known mRNA detection methods in the art may be adapted for use in determining the level of mRNA of a gene of interest.

An alternative method for determining the level of expression of a gene of interest in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques wellknown in the art. These detection schemes are especially useful for the detection of nucleic acid molecules if such molecules are present in very low numbers. In particular aspects of the disclosure, the level of expression of a gene of interest is determined by quantitative fluorogenic RT-PCR (i.e., the TAQMANTm System) or the DUAL-GLO® Luciferase assay.

The expression levels of mRNA of a gene of interest may be monitored using a membrane blot (such as used in hybridization analysis such as northern, Southern, dot, and the like), or microwells, sample tubes, gels, beads or fibers (or any solid support including bound nucleic acids). See U.S. Pat. Nos. 5,770,722; 5,874,219; 5,744,305; 5,677,195; and 5,445,934, which are incorporated herein by reference. The determination of gene expression level may also include using nucleic acid probes in solution.

The level of mRNA expression may also be assessed using branched DNA (bDNA) assays or real time PCR (qPCR). The use of this PCR method is described and exemplified in the Examples presented herein. Such methods can also be used for the detection of nucleic acids of the gene of interest.

Furthermore, the level of GluK2 protein produced by the expression the Grik2 gene may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest. Additionally, the above assays may be used to report a change in the mRNA sequence of interest that results in the recovery or change in protein function thereby providing a therapeutic effect and benefit to the subject, treating a disorder in a subject, and/or reducing of symptoms of a disorder in the subject.

Accordingly, the aforementioned assays for measuring Grik2 mRNA or GluK2 protein expression may be used to identify a subject (e.g., a subject suffering from an epilepsy, such as, e.g., TLE) as being in need of therapeutic treatment with one or more ASO agents disclosed herein (e.g., any one of the ASO agents described in Table 2) or a nucleic acid vector encoding the same. For example, a patient identified as having TLE may exhibit an epileptogenic focus within the temporal lobe of one hemisphere of the brain that causes uncontrollable (e.g., treatment-resistant, e.g., chronic) seizures. As is discussed herein, such an epileptogenic focus may result from, e.g., aberrant sprouting of recurrent dentate granule cell mossy fibers and abnormal (i.e., increased) expression of Grik2 at the recurrent synapses formed by said mossy fibers. Using the assays described above, a determination can be made as to whether the subject would benefit from a therapy using one or more of the Grik2 ASO agents disclosed herein, e.g., by performing a small biopsy on brain tissue collected from the hippocampus of the epileptogenic hemisphere and from the same region in the healthy hemisphere. A showing that the epileptogenic hemisphere exhibits higher (e.g., by at least 5%, 10%, 15%, 20%, 25%, 30%, 35%, 40%, 45%, 50%, 30 55%, 60%, 65%, 70%, 75%, 80%, 85%, 90%, 91%, 92%, 93%, 94%, 95%, 96%, 97%, 98%, 99%, or more) levels of expression of Grik2 mRNA or GluK2 protein as compared to the unaffected hemisphere would indicate that the patient may benefit from therapy using the methods and compositions disclosed herein. In the case that the subject with TLE presents with an epileptogenic focus in both brain hemispheres, Grik2 mRNA or GluK2 protein levels could be compared between hippocampal tissue obtained from one or more hemispheres from the TLE-afflicted subject and hippocampal tissue from the same hemisphere(s) of a healthy control subject (e.g., from post-mortem tissue of a subject without TLE) using the assays disclosed above. A showing that the epileptogenic hemisphere(s) of the TLE-afflicted subject exhibits higher (e.g., by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more) expression of Grik2 mRNA or GluK2 protein as compared to the same hemisphere(s) of a healthy subject would indicate that the TLE-afflicted subject would benefit from therapeutic treatment with the disclosed compositions and methods. Grik2 mRNA levels or GluK2 protein levels in the neuronal cells of a subject suspected to be in need of treatment can also be compared to standard or reference levels of these analytes that are known to indicate a disease state.

In addition, the assays described above may be utilized to determine whether a subject (e.g., a subject suffering from an epilepsy, such as, e.g., TLE) has responded to treatment using the compositions and methods disclosed herein. For example, as is discussed above, hippocampal brain tissue from an epileptogenic brain hemisphere(s) can be obtained from the TLE-afflicted subject by way of a small biopsy prior to treatment with the compositions and methods disclosed herein and expression of Grik2 mRNA or GluK2 protein may be assessed using the aforementioned assays. The subject may then be administered treatment according to the methods and compositions disclosed herein. Subsequent to the recovery of the patient following treatment (e.g., 1, 5, 10, 15, 30, 60, 90, or more days after treatment) with the disclosed methods and compositions, a second biopsy may be performed over the same brain regions assessed prior to treatment and levels of Grik2 mRNA or GluK2 protein may again be assessed. A showing that the TLE-afflicted subject exhibits lower (e.g., by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more) levels of expression of Grik2 mRNA or GluK2 protein would indicate that the subject was responsive to treatment. Alternatively, Grik2 mRNA or GluK2 protein levels may be compared with respect to expression of the same from one or more healthy control subjects. A showing that Grik2 mRNA or GluK2 protein levels in the TLE-afflicted patient after treatment are statistically indistinguishable from levels of the same in one or more healthy control subjects would indicate that the patient is responsive to treatment. Grik2 mRNA levels or GluK2 protein levels in the neuronal cells of a treated subject can also be compared to standard or reference levels of these analytes that are known to indicate the absence of a disease state.

### Methods of Treatment

A subject with epilepsy (e.g., TLE) may be treated using the compositions and methods described herein. The compositions (e.g., an ASO agent-containing composition or a vector containing the same) may be administered as a preventative treatment to a subject in need thereof (e.g., a subject diagnosed as having or being at risk of having an epilepsy (e.g., TLE). A subject at risk of developing an epilepsy may show early symptoms of an epilepsy or may not yet be symptomatic when treatment is administered.

Routes of Administration

The compositions disclosed herein may be administered to a subject (e.g., a subject identified as having TLE) using standard methods. For example, the compositions disclosed herein can be administered by any of a number of different routes including, e.g., systemic administration. Non-limiting examples of systemic administration include enteral (e.g., oral) or parenteral (e.g., intravenous, intra-arterial, transmucosal, intraperitoneal, epicutaneous, intramucosal (e.g., intranasal or sublingual), intramuscular, or transdermal) administration. Additional routes of administration may include intradermal, subcutaneous, and percutaneous injection.

The compositions disclosed herein may also be administered using methods suitable for local delivery of ASO agents or nucleic acid vectors encoding the same. Non-limiting examples of local administration include epicutaneous (e.g., topical), intra-articular, and inhalational routes. In particular, the disclosed compositions may be locally administered to brain tissue (e.g., neural cells, such as e.g., neurons and/or astroglia)) of the subject.

In particular, the ASO agents and nucleic acid vectors encoding the same may be administered locally to brain tissue of the subject, such as brain tissue determined to exhibit increased epileptiform activity. Local administration to the brain generally includes any method suitable for delivery of an ASO agent or a nucleic vector encoding the same to brain cells (e.g., neural cells), such that at least a portion of cells of a selected, synaptically connected cell population is contacted with the composition. Vectors may be delivered to any cells of the CNS, including neurons, astroglia, or both. Generally, the vector is delivered to the cells of the CNS, including, e.g., cells of the spinal cord, brainstem (medulla, pons, and midbrain), cerebellum, diencephalon (e.g., thalamus and hypothalamus), telencephalon (corpus striatum, cerebral cortex (e.g., cortical regions in the occipital, temporal, parietal, or frontal lobes), or combinations thereof, or any suitable subpopulation of cells therein. Further sites for delivery include the red nucleus, amygdala, entorhinal cortex ,and neurons in ventrolateral or anterior nuclei of the thalamus.

The vectors of the disclosure may be delivered by way of stereotactic injections or microinjections directly into the parenchyma or ventricles of the CNS. In a particular example, the vectors of the disclosure may be delivered directly to one or more epileptic foci in the brain of the subject. For example, the subject may be administered a vector of the disclosure by means of a stereotactic injection directly into one or both hemispheres of the allocortex (e.g., hippocampus) or neocortex (e.g., frontal lobe). In a particular example, the subject is administered a vector of the disclosure by means of a stereotactic injection directly into one or both hemispheres of the hippocampus. Alternatively, the vectors of the disclosure may be administered by intravenous injection, for example in the context of vectors that exhibit tropism for CNS tissues, including but not limited to AAV9 or AAVrh10.

To deliver a vector of the disclosure specifically to a particular region and to a particular population of CNS cells, the vector may be administered by stereotaxic microinjection. For example, subjects may have a stereotactic frame base surgically fixed in place (screwed into the skull). The brain with a stereotactic frame base (e.g., MRI compatible stereotactic frame base with fiducial markings) is imaged using high resolution MRI. The MRI images are then transferred to a computer which runs stereotactic software. A series of coronal, sagittal and axial images are used to determine the target injection site and trajectory of the cannula or injection needle used for injecting a composition of the disclosure into the brain. The software directly translates the trajectory into three-dimensional coordinates appropriate for the stereotactic frame. Holes are drilled above the entry site and the stereotactic apparatus is positioned with the injection needle implanted at the given depth. The composition (such as a composition disclosed herein) may be injected at the target sites. In the case that the composition includes an integrating vector, rather than producing viral particles, the spread of the vector is minor and mainly a function of passive diffusion from the site of injection. The degree of diffusion may be controlled by adjusting the ratio of vector to fluid carrier.

Additional routes of administration may also include local application of the vector under direct visualization, e. g., superficial cortical application, or other non-stereotactic application. The vector may be delivered intrathecally (e.g., directly into the cisterna magna), in the ventricles (e.g., using intracerebroventricular (ICV) injection) or by intravenous injection.

In one example, the method of the disclosure includes intracerebral or intracerebroventricular administration through stereotaxic injections. However, other known delivery methods may also be adapted in accordance with the disclosure. For example, for a more widespread distribution of the composition across the CNS, it may be injected into the cerebrospinal fluid, e.g., by lumbar puncture. To direct the composition to the peripheral nervous system, it may be injected into the spinal cord, one or more peripheral ganglia, or under the skin (subcutaneously or intramuscularly) of the body part of interest.

In certain situations, the composition can be administered via an intravascular approach. For example, the composition can be administered intra-arterially (carotid) in situations where the blood-brain barrier is disturbed or not disturbed. Moreover, for more global delivery, the composition can be administered during the “opening” of the blood-brain barrier achieved by infusion of hypertonic solutions including mannitol.

The most suitable route for administration in any given case will depend on the particular composition administered, the subject, the particular epilepsy being treated, pharmaceutical formulation methods, administration methods (e.g., administration time and administration route), the subjects age, body weight, sex, severity of the diseases being treated, the subjects diet, and the subject's excretion rate.

Combination Therapy

The compositions disclosed herein may be administered to a subject in need thereof (e.g., a human subject) to treat an epilepsy (e.g., a TLE) in combination with one or more additional therapeutic modalities (e.g., 1, 2, 3, or more additional therapeutic modalities), including other therapeutic agents or physical interventions (e.g., rehabilitation therapy or surgical intervention). The two or more agents can be administered at the same time (e.g., administration of all agents occurs within minutes, minutes, minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). Sequential or substantially simultaneous administration of each therapeutic agent can be performed by any appropriate route including, but not limited to, oral routes, intravenous routes, intramuscular routes, local routes, and direct absorption through mucous membrane tissues. The therapeutic agents can be administered by the same route or by different routes. For example, a first therapeutic agent of the combination may be administered by intravenous injection while a second therapeutic agent of the combination can be administered locally in a compound-impregnated microcassette. The first therapeutic agent may be administered immediately, up to 1 hour, up to 2 hours, up to 3 hours, up to 4 hours, up to hours, up to 6 hours, up to 7 hours, up to, 8 hours, up to 9 hours, up to hours, up to 11 hours, up to 12 hours, up to 13 hours, 14 hours, up to hours 16, up to 17 hours, up18 hours, up to 19 hours up to hours, up to 21 hours, up to 22 hours, up to 23 hours up to 24 hours or up to 1-7, 1-14, 1-21 or 1-days before or after the second therapeutic agent.

In cases in which the subject is diagnosed as having or at risk of developing an epilepsy (e.g., a TLE), the second therapeutic agent may include one or more antiepileptic drug (AED) including, but not limited to valproate, lamotrigine, ethosuximide, topiramate, lacosamide, levetiracetam, clobazam, stiripentol, benzodiazepine, phenytoin, carbamazepine, primidone, phenobarbital, gabapentin, pregabalin, tiagabine, zonisamide, felbamate, and/or vigabatrin. In some cases, the second therapeutic modality may be surgical intervention, such as, e.g., surgical resection of the epileptogenic brain region (e.g., a temporal lobe resection) using methods well-known in the art, such as, e.g., radiosurgery (e.g., gamma knife or laser ablation). Additional therapeutic modalities that can be administered together with the methods and compositions of the present disclosure include vagus nerve stimulation, deep brain stimulation, transcranial magnetic stimulation, and ketogenic diet.

In particular examples, the subject may be administered an immune suppressant, including a regimen of corticosteroid alone, or tacrolimus or rapamycin (sirolimus), e.g., in combination with mycophenolic acid or in combination with a corticosteroid such as prednisolone and/or methylprednisolone. Other immune suppression regimens well known in the art can be employed in conjunction with the methods and compositions of the disclosure. Such immune suppression treatments may be administered before, after, or concomitantly with the gene therapy.

Dosing

Subjects that can be treated as described herein are subjects diagnosed as having or at risk of developing an epilepsy (e.g., a TLE). A subject that can be treated using the disclosed methods and compositions include, e.g., a subject who has had one or more previous therapeutic interventions related to the treatment of epilepsy or a subject who has had no previous therapeutic intervention for treatment of an epilepsy.

The ASO agent of the disclosure may be administered in an amount and for a time effective to result in one or more of (e.g., 2 or more, 3 or more, 4 or more of): (a) decrease the level of Grik2 mRNA and/or GluK2 protein in a cell of the subject, (b) delayed onset of the disorder, (c) increased survival of subject, (d) increased progression free survival of a subject, (e) recovery or change in GluK2 protein function, (f) reduce risk of seizure recurrence; (g) reduction of excitotoxicity and associated neuronal cell death in the CNS; (h) restoration of a physiological excitation-inhibition balance in the affected region of the CNS (e.g., the hippocampus); and/or (i) reduction in one or more symptoms of a epilepsy (e.g., frequency, duration, or intensity of epileptic seizures, weakness, absence, sudden confusion, trouble understanding or producing speech, cognitive impairment, impaired mobility, dizziness, or loss of balance or coordination, paralysis, and emotional dysregulation).

Accordingly, the present disclosure relates to a method for treating an epilepsy (e.g., TLE) in a subject in need thereof, in which the method includes administering an effective amount of a vector including an oligonucleotide encoding an inhibitory RNA (e.g., an ASO, such as, e.g., siRNA, shRNA, miRNA, or shmiRNA, or shmiRNA) that binds specifically to Grik2 mRNA and inhibits expression of GluK2 protein in the subject. In particular, the invention provides a method of treating an epilepsy in a subject in need thereof including administering to the subject a therapeutically effective amount of an ASO agent disclosed herein or a nucleic acid vector encoding the same.

The epilepsy to be treated utilizing the disclosed methods and compositions may be TLE (e.g., mTLE or ITLE), benign Rolandic epilepsy, a frontal lobe epilepsy, infantile spasms, a juvenile myoclonic epilepsy, a juvenile absence epilepsy, a childhood absence epilepsy (pyknolepsy), a hot water epilepsy, Lennox-Gastaut syndrome, Landau-Kleffner syndrome, Dravet syndrome, a progressive myoclonus epilepsy, a reflex epilepsy, Rasmussen's syndrome, a limbic epilepsy, status epilepticus, an abdominal epilepsy, massive bilateral myoclonus, a catamenial epilepsy, Jacksonian seizure disorder, Lafora disease, and a photosensitive epilepsy. For example, a subject may be diagnosed with a TLE (e.g., mTLE or ITLE), such as, a TLE characterized by focal or generalized seizures. In some cases, the epilepsy may be a chronic epilepsy, such as, e.g., an epilepsy that is refractory to treatment (i.e., a pharmaco-resistant epilepsy, such as a pharmaco-resistant TLE).

For the treatment of epilepsy and to ameliorate the symptoms of seizures and epileptiform discharges as is discussed herein, a useful polynucleotide may be deployed by a vector which encodes a functional RNA, e.g., siRNA, shRNA, miRNA, or shmiRNA, that inhibits the expression of Grik2 mRNA.

The disclosed compositions can be administered in amounts determined to be appropriate by those of skill in the art. In some cases, the rAAV is administered at a dose of 10⁵, 10⁶, 10′, 10⁸, 10⁹, 10¹⁰ 10¹¹, 10¹², 10¹³, 10¹⁴, or 10¹⁵genome copies (GC) per subject. In some embodiments the rAAV is administered at a dose of 10⁵, 10⁶, 10′, 10⁸, 10⁹, 10¹⁰, 10¹¹, 10¹², 10¹³, or 10¹⁴ GC/kg (total weight of the subject).

In some cases, 1×10¹² to x 10¹⁴ GC are administered. In some cases, a flat dose of 1×10¹² to 5×10¹⁴ GC is administered to a pediatric patient or an adult patient.

In some cases, dosages are measured by the number of GC administered to the cerebrospinal fluid (CSF) of the patient (e.g., injected intrathecally, e.g., via suboccipital puncture or lumbar puncture) per gram of the patient's brain mass. In some cases, 10⁵, 10⁶, 10′, 10⁸, 10⁹, 1 0^{9,}10¹° , 10¹¹, 10¹², 1 0¹³, 10¹⁴, or 10¹⁵ genome copies per gram of patient's brain mass are administered. In some cases, 1×10⁵ genome copies per gram of patient's brain mass are administered. In some cases, 1×10⁶ genome copies per gram of patient's brain mass are administered. In some cases, 1×10′ genome copies per gram of patient's brain mass are administered. In some cases, 1×10⁸ genome copies per gram of patient's brain mass are administered. In some cases, 1×10⁹ genome copies per gram of patient's brain mass are administered. In some cases 1×10¹⁰ genome copies per gram of patient's brain mass are administered. In some cases x 10¹⁰ genome copies per gram of patient's brain mass are administered. In some cases 1×10⁹ to 1×10¹¹ genome copies per gram of patient's brain mass are administered. In some cases 1×10⁹ to x 10¹⁰ genome copies per gram of patient's brain mass are administered. In some cases 2×10⁹ to 9×10¹⁰ genome copies per gram of patient's brain mass are administered. In some cases x 10⁹ to 1×10¹¹ genome copies per gram of patient's brain mass are administered. In other cases, 1×10¹⁰ to x 10¹⁰ genome copies per gram of patient's brain mass are administered. In other embodiments, 1×10¹⁰ to 9×10¹⁰ genome copies per gram of patient's brain mass are administered. The patient's (subject's) brain weight estimation is obtained from an MRI brain volume determination, which is converted to brain mass and utilized to calculate a precise dose of drug administered. Brain weights may also be estimated based on age range, using a published database.

Optionally, the disclosed agents may be administered as part of a pharmaceutically acceptable composition suitable for delivery to a subject, as is described herein. The disclosed agents are included within these compositions in amounts sufficient to provide a desired dosage and/or elicit a therapeutically beneficial effect, as can be readily determined by those of skill in the art.

The disclosed compositions described herein may be administered in an amount (e.g., an effective amount) and for a time sufficient to treat the subject or to effect one of the outcomes described above (e.g., a reduction in one or more symptoms of disease in the subject). The disclosed compositions may be administered once or more than once. The disclosed compositions may be administered once daily, twice daily, three times daily, once every two days, once weekly, twice weekly, three times weekly, once biweekly, once monthly, once bimonthly, twice a year, or once yearly. Treatment may be discrete (e.g., an injection) or continuous (e.g., treatment via an implant or infusion pump). Subjects may be evaluated for treatment efficacy 1 week, 2 weeks, 1 month, 2 months, 3 months, 4 months, months, 6 months or more following administration of a composition of the disclosure depending on the composition and the route of administration used for treatment. Methods of evaluating treatment efficacy are disclosed herein (see, e.g., “Pharmaceutical Uses”). Depending on the outcome of the evaluation, treatment may be continued or ceased, treatment frequency or dosage may change, or the patient may be treated with a different disclosed composition. Subjects may be treated for a discrete period of time (e.g., 1, 2, 3, 4, 5, 6, 7, 8, 9, 10, 11, or 12 months) or until the disease or condition is alleviated, or treatment may be chronic depending on the severity and nature of the disease or condition being treated. For example, a subject diagnosed with TLE and treated with a composition disclosed herein may be given one or more (e.g., 1, 2, 3, 4, 5, 6, 7, 8, 9, 10, or more) additional treatments if initial or subsequent rounds of treatment do not elicit a therapeutic benefit (e.g., reduction of any one of the symptoms disclosed herein or a reduction in the levels of Grik2 mRNA or GluK2 protein levels in the afflicted brain region of the subject).

### Kits

The disclosure also provides kits that include a composition disclosed herein that inhibits the expression of a Grik2 gene in a subject (e.g., an ASO targeting a Grik2 mRNA) for use in the prevention or treatment of an epilepsy (e.g., a TLE, such as treatment-refractory TLE). The kits can optionally include an agent or device for delivering the composition to the subject. In other examples, the kits may include one or more sterile applicators, such as syringes or needles. Further, the kits may optionally include other agents, e.g., anesthetics or antibiotics. The kit can also include a package insert that instructs a user of the kit, such as a physician, to perform the methods disclosed herein.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example1A

### Grik2 mRNA-targeting antisense oligonucleotide design and selection

Design of Grik2 mRNA-targeting antisense oligonucleotides

In order to develop antisense oligonucleotides (ASOs) capable of targeting and inhibiting the expression of Grik2 mRNA, a collection of Grik2 mRNA sequences (5′ untranslated region (UTR), coding region (CDS), and 3′ UTR) was obtained from the NCBI GenBank database from Homo sapiens as well as other animal model species, including Mus musculus, Rattus norvegicus, and Macaca mulatta. Focus was placed on the longest transcript variant (i.e., SEQ ID NO: 115), but other transcript variants were included in the collection. Sequences evaluated include: SEQ ID NO: 115 (NM_021956 (H. sapiens)), SEQ ID NO: 117 (NM_175768 (H. sapiens)), SEQ ID NO: 118 (NM_001166247 (H. sapiens)), SEQ ID NO: 119 (NM_001111268 (M. musculus)), SEQ ID NO: 120 (NM_010349 (M. musculus)), SEQ ID NO: 121 (NM_001358866 (M. musculus)), SEQ ID NO: 122 (XM_015136995 (M. mulatta)), SEQ ID NO: 123 (XM_015136997 (M. mulatta)), SEQ ID NO: 124 (NM_019309.2 (R. norvegicus)). A sequence alignment was performed using the sequence alignment program MUSCLE to identify regions of significant sequence homology between species, and guide RNA (i.e., ASO sequence involved in complementary base-pairing with the Grik2 mRNA) design preference was given to these regions in the interest of increasing the likelihood of a given guide sequence to effectively target the desired transcript in each species of interest throughout the therapeutic development process.

The literature suggests that secondary structure of mRNA can have a significant impact on the efficiency of targeting by RNA interference (RNAi)—regions that do not contain secondary structure are more accessible to the RNAi machinery and are therefore hypothesized to be more efficiently targeted. Conversely, regions of the mRNA that are predicted to participate in base pairing, forming an RNA-RNA duplex, are less accessible to the RNAi machinery and are hypothesized to be less efficiently targeted by RNAi approaches. The secondary structure of Grik2 mRNA is not characterized for any species. Thus, a strategy was devised to use predictive software that employs an algorithm based on either reducing the free energy (i.e., minimum free energy structure, or MFE) of the folded RNA or reducing the base pair distance compared to all other possible folded orientations (centroid). This assessment was performed on Grik2 mRNA variants 1 from Homo sapiens, Mus musculus, and Macaca mulatta using the RNAfold WebServer (rna.tbi.univie.ac.at//cgi-bin/RNAWebSuite/RNAfold.cgi). Regions of each transcript with low base pairing probability and/or high positional entropy (colored yellow through blue on FIG. 1A) were identified and prioritized for guide design. These regions included clearly delineated loop regions (designated as Arabic numerals 1-14 in FIG. 1B, which correspond to SEQ ID NOs: 145-158, respectively) as well as regions depicted as stem-like regions but with low probability of base pairing (designated by Arabic numerals 15-19 in FIG. 1B, which correspond to SEQ ID NOs: 159-163, respectively). Priority was given to regions that were predicted to have low base pairing probability and/or high positional entropy in more than one species. An alignment of various identified RNAi guide sequences to Grik2 mRNA (SEQ ID NO: 115) is shown in FIGS. 1C-1G.

Regions of interest in the Grik2 mRNA selected by the above listed methods were then evaluated in silico to predictively determine the likelihood that small inhibitory RNAs designed to target these regions also have the potential to target other transcripts, resulting in undesired off-target effects based on mis-regulation of non-target genes. To predictively evaluate the probability of off-target effects mediated by guide RNAs designed against a particular region on the Grik2 transcripts, the online tool siSPOTR (“sRNA Sequence Probability-of-Off-Targeting Reduction” available at sispotr.icts.uiowa.edu/sispotr/index.html) was used. After entering a transcript of interest, this program identifies a list of siRNAs or shRNAs (either of which could be converted to synthetic ASOs and delivered as such) that are predicted to target the transcript of interest while minimizing the likelihood that the siRNAs or shRNAs will target other transcripts. siSPOTR also indicates if the seed sequence (e.g., the sequence involved in complementary base pairing with the target DNA or mRNA sequence, such as a Grik2 mRNA sequence) from a suggested small RNA molecule also corresponds to the seed sequence of a known, endogenously expressed microRNA (miRNA), suggesting that the candidate molecule could mis-regulate a pathway normally regulated by the native miRNA. These candidates were avoided. As was the case for the use of the RNAfold WebServer, transcript variants 1 from Homo sapiens,

Mus musculus, and Macaca mulatta were evaluated using siSPOTR, both against the human and murine transcriptome. This allowed for the identification of candidate small RNAs derived from each transcript that were predicted to have a low likelihood of targeting mRNAs either in human cells (e.g., cell lines for in vitro or human resections for ex vivo target validation) or in murine cells (e.g., in vivo murine models of efficacy). This also allowed for identification of candidate small RNAs that were homologous in the sequences they targeted in all three input transcripts that also were predicted to cause minimal off-target effects in both human and murine cells, which is of particular interest for minimizing both the variation in efficacy and the off-target profile across various models during the process of drug development.

Finally, for the purpose of screening candidate target sequences using synthetic RNA guides in the luciferase reporter system, the corresponding 21 bp target sequence was evaluated using BLASTn for “Somewhat similar sequences” (available at blast.ncbi.nlm.nih.gov/Blast.cgi?PAGE_TYPE=BIastSearch) in order to determine if any RNAs expressed from the human genome bore significant similarity (e.g., >90%, then >80%) to the proposed target and would therefore have the possibility of being targeted by the guide designed against Grik2.

### In Vitro Screen of Gluk2 Antisense Oligonucleotides Using a Cell Line Reporter Assay

The sequence for the human Grik2 (transcript variant 1) mRNA (including 5′ and 3′ UTRs) was obtained from the NCBI GenBank (NM_021956.4; SEQ ID NO: 115), synthesized and cloned into the 3′ UTR of firefly luciferase under the control of the phosphoglycerate kinase (PGK) promoter in the context of the pMIR-GLO (Promega, Catalog No. E1330) dual luciferase reporter plasmid, termed pmiR-GLO-hGluK2. Therefore, expression from the PGK promoter generated a hybrid firefly luciferase-human Grik mRNA that allowed for the evaluation of small RNA guides targeting human Grik2 mRNA, as RNAi-mediated reduction in Grik2 mRNA expression also resulted in a reduction of the firefly luciferase (ffluc) reporter protein. Luciferase can be quantified by a variety of methods well known in the art. This reporter plasmid also encodes a renilla luciferase reporter under the control of the SVpromoter, whose expression served as a normalization control, as it does not contain any targeting sequences of interest in its 3′ UTR. The ratio of ffluc:renilla signal (expressed in RLU) was then reported and compared to the ratio obtained when an irrelevant small RNA guide (used as a negative control) is evaluated in the same fashion. The results of Grik2 mRNA knockdown was therefore reported as a “% GluK2 knockdown” (also referred to as % KD), and any non-specific effect on GluK2 protein expression was measured as “% residual expression” relative to the ratios obtained from the negative control, as follows.

Here, 5ng pmiR-GLO-hGluK2 was co-transfected into 96 well plates of WT 293T cells with a small RNA guide, in this particular case, pg of a synthetic siRNA designed against Grik2 mRNA or an irrelevant negative control ASO. As an additional control and to determine if transfection of the ASO itself had a more generic, global effect on protein expression, each ASO was additionally co-transfected (in separate wells) with an empty pmiR-GLO plasmid that does not contain the Grik2 mRNA sequence in the 3′ UTR of luciferase and therefore does not produce the ffluc-hGluK2 hybrid mRNA. 48 hours after transfection, cells were lysed and both firefly and renilla luciferase expression were determined using the Dual-Glo Luciferase Assay System (Promega) and reported in relative light units (RLUs). As is discussed above, the ratio of ffluc:renilla luciferase produced in wells receiving experimental ASOs against Grik2 mRNA in combination with the pmiR-GLO-hGluK2 reporter was calculated, was compared to the ratio obtained from wells receiving the irrelevant, negative control siRNA, and was reported as “percent knockdown” (% KD) of the target mRNA, in this case ffluc-hGluK2 (FIG. 1H; Table 2). These values were used to determine which small RNA guides efficiently targeted the Grik2 mRNA sequence.

Additionally, the ratio of ffluc:renilla luciferase produced in wells receiving experimental ASO agents against Grik2 in combination with the pmiR-GLO-empty reporter was calculated, compared to the ratio obtained from wells receiving the irrelevant negative control ASO, and was reported as “percent residual ffluc expression” from the non-targeted ffluc mRNA (FIG. 11). These values were used to determine which small RNA guides had a non-specific effect on protein expression in transfected cells, using the non-targeted ffluc as a surrogate for protein expression. These values were plotted for each guide candidate to identify guides that significantly reduced ffluc-hGluK2 mRNA expression but that did not interfere with non-targeted ffluc expression in a non-specific fashion (FIG. 1J). From this plot, cutoffs were established for guide selection, i.e., >66% GluK2 knockdown and >66% residual non-targeted ffluc expression (dotted lines on graph).

### Example1B

### Thermodynamic Properties of Grik2 Target:siRNA Duplexes

RNA-RNA interactions and their thermodynamic properties were predicted using the RNAup WebServer, as depicted in Tables and 11 below. 19 base pair (bp) siRNAs (having 19 bases of homology with GluK2 mRNA) and 21 base pair Grik2 siRNAs (with 21 base guides having 19 bases of homology with GluK2 mRNA) were queried to assess thermodynamic properties by RNAup, as follows:

Total Free Energy of Binding, Energy from Duplex Formation, and Target Opening Energy (Opening Energy Grik2 mRNA), and these calculations were compared to the determination of percent knockdown (% KD, see Example 1A) and GC% for each guide in order to correlate favorable guide properties. RNAup calculated the thermodynamics of RNA—RNA interactions in essentially two stages. First, the probability that a potential binding site remains unpaired (e.g. the free energy needed to open the target site) is computed, then this “accessibility” is combined with the interaction energy to obtain the total binding energy.

TABLE 10
Thermodynamic property calculations for 19-bp synthetic RNA guides

| | | | | | Total free | | | |
|---|---|---|---|---|---|---|---|---|
| | | | | | energy of | Energy from | Opening | |
| | | % KD for the | | | binding | duplex formation | energy | |
| siRNA | SEQ ID | corresponding | | %GC | (kcal/mol) | (kcal/mol) | GluK2 mRNA | |
| ID | NO | 19 bp guide | Guide sequence (19 base) | (19 bp) | (19 bp guide) | (19 bp guide) | (kcal/mol) | Grik2 Target Region |
| MD | 70 | 0*** | CAUUCCCAUAGCUAAUGCC | 47.4 | -24.33* | -34.51 | 9.12* | Exon 3 |
| G5 | 15 | 89 | UGAUAUUCCAUCUCCUGGC | 47.4 | -28.88** | -35.12* | 5.36 | 3'UTR |
| | | | | | | | | Centroid Unpaired 3 |
| | | | | | | | | Exon 16 |
| Y9 | 88 | 87 | CAUAGGUAAUAGCCAGUGG | 47.4 | -23.71 | -33.91 | 6.64 | Exon 5 |
| TO | 14 | 86 | CGUUCCACUGUCAGAAAGG | 52.6* | -27.58* | -33.3 | 5.36 | Exon 13 |
| | | | | | | | | Centroid Unpaired 1 |
| TK | 74 | 85 | AGUCGAUGACCUUCUCUCG | 52.6* | -25.77* | -35* | 8.16* | 5' UTR |
| | | | | | | | | Exon 1 |
| TL | 20 | 85 | UGUCGAUUACACUGCAAGG | 47.4 | -26.52* | -33.4 | 6.66 | Exon 7 |
| GI | 77 | 83 | GUCUCGAUAUGGAGAACCC | 52.6* | -25.35* | -37.02* | 7.22 | overlaps Exon 3 and |
| | | | | | | | | Exon 4 |
| G0 | 1 | 83 | AAGGGUAGUAUUGGGUAGC | 47.4 | -32.78** | -34.52 | 17.24** | Exon 2 |
| | | | | | | | | Centroid Loop 1 |
| TT | 4 | 83 | UGUUAACACCACUGUAUCG | 42.1 | -26.56* | -31.41 | 4.49 | Exon 6 |
| | | | | | | | | Centroid Loop 2 |
| TP | 13 | 82 | uGUUCCACUGUCAGAAAGG | 47.4 | -27.57* | -33.3 | 5.36 | Exon 13 |
| | | | | | | | | Centroid Unpaired 1 |
| MU | 96 | 82 | UUCAUAUGUAAAGCCAAGG | 36.8 | -25.88* | -29.81 | 3.88 | Exon 10 |
| | | | | | | | | Centroid Loop 5 |
| Y5 | 60 | 81 | UUGAGUCGAAGAUUAUACC | 36.8 | -23.74 | -29.91 | 5.79 | 5' UTR |
| | | | | | | | | Exon 1 |
| TJ | 21 | 80 | GAUGCGUCGAGUGGUGACC | 63.2** | -27.16* | -39.7** | 10.53** | Exon 11 |
| ML | 85 | 80 | AGAGCUCCAACUCCAAACC | 52.6* | -24.8* | -35.72* | 9.63* | Exon 11 |
| CW | 41 | 80 | AUAAACGCAGUCCACUUCC | 47.4 | -24.29* | -32.82 | 8.51* | Exon 7 |
| Y8 | 87 | 80 | uAUAGGUAAUAGCCAGUGG | 42.1 | -23.25 | -32.31 | 6.64 | Overlaps Exon 4 and |
| | | | | | | | | Exon 5 |
| MW | 80 | 79 | CAGAGCAUUGCAGAUGGAC | 52.6* | -26.29* | -36.8* | 9.82* | Exon 4 |
| TM | 17 | 78 | UAUUUCCUUGAUAAUUGGC | 31.6 | -23.42 | -27.73 | 3.91 | 3' UTR |
| G9 | 68 | 77 | CCCAUAGCUAAUGCCUGUU | 47.4 | -27.7* | -34.91 | 5.54 | Overlaps with Exon |
| | | | | | | | | 14 and 15 |
| ME | 69 | 77 | uUGUCAUCAUUCCCAUAGC | 42.1 | -21.76 | -32 | 10.17** | Exon 14 |
| GE | 65 | 76 | UUAGUCGGAGAGCAUCCGG | 57.9* | -26.97* | -37.8* | 5.42 | Exon 13 |
| | | | | | | | | Centroid Unpaired 1 |
| G6 | 18 | 76 | uAUCCUCAAUUCUUCUACC | 36.8 | -24.73* | -30.02 | 5.29 | Exon 16 |
| MH | 94 | 74 | uGUCAUAAAUCCAUCCAGC | 42.1 | -24.6* | -31.82 | 7.21 | Exon 6 |
| TN | 16 | 73 | AUAAGUGAUGAACAUCUGC | 36.8 | -25.22* | -29.71 | 3.91 | 3'UTR |
| | | | | | | | | Centroid Unpaired 4 |
| | | | | | | | | Exon 16 |
| TU | 3 | 72 | AGCAUUGCAGAUGGACUGC | 52.6* | -22.63 | -35.92* | 10.24** | Exon 3 |
| GD | 7 | 72 | UGUAGAUAACUCUCUGAGG | 42.1 | -26.46* | -32.9 | 5.08 | Exon 10 |
| | | | | | | | | Centroid Loop 5 |
| MG | 95 | 71 | uAUCAGUCGUCAUAAAUCC | 36.8 | -23.67 | -29.32 | 5.64 | Exon 6 |
| TR | 11 | 71 | UUAUUAUUUGGGAUAUGGG | 31.6 | -26.86* | -28.01 | 0.88 | 3'UTR |
| | | | | | | | | Centroid Loop 12 |
| | | | | | | | | Exon 16 |
| TH | 22 | 70 | CUCGAACAUAGGUAAUAGC | 42.1 | -23.63 | -31.62 | 7.86* | 5' UTR |
| | | | | | | | | Exon 1 |
| CL | 30 | 69 | uAGCGGGUCUGUAUGUGGG | 57.9* | -28.93** | -38.4* | 8.84* | 3' UTR |
| | | | | | | | | Exon 16 |
| MF | 93 | 69 | UAGAGACUGACAUAGAAGG | 42.1 | -23.86 | -32.6 | 8.39* | Exon 3 |
| XU | 51 | 68 | UGUCUCGAUAUGGAGAACC | 47.4 | -22.97 | -34.42 | 7 | 5' UTR |
| | | | | | | | | Exon 1 |
| XY | 83 | 67 | CACCACGUCUGAGUCAGGG | 63.2** | -29.29** | -40.7** | 9.92* | Exon 6 |
| XX | 82 | 67 | uACCACGUCUGAGUCAGGG | 57.9* | -27.65* | -37.6* | 8.47* | Exon 6 |
| YA | 63 | 67 | GGUUGCAUAUUUCCACAGG | 47.4 | -27.06* | -34.01 | 6.42 | Overlaps Exon 4 and |
| | | | | | | | | Exon 5 |
| GG | 91 | 67 | AGUCGUCAUAAAUCCAUCC | 42.1 | -24.54* | -31.82 | 7.28 | Exon 11 |
| CP | 34 | 66* | uUCGAUGGUUGUUGACUCC | 47.4 | -24.97* | -33.12 | 6.48 | Exon 3 |
| G8 | 92 | 66* | ACUGACAUAGAAGGAAUCU | 36.8 | -21.89 | -30.51 | 8.19* | Overlaps with Exon |
| | | | | | | | | 14 and 15 |
| CX | 42 | 65* | GACCGCAGACACGAUCACG | 63.2** | -28.01** | -39.7** | 10.48** | 5' UTR |
| | | | | | | | | Exon 1 |
| G1 | 5 | 64* | AAUCGGGUUUCGGAGGUGC | 57.9* | -30.11** | -36.52* | 5.58 | Exon 6 |
| | | | | | | | | Centroid Loop 3 |
| GF | 64 | 63* | UGCGUCGAGUGGUGACCGC | 68.4** | -26.88* | -41.02** | 10.61** | Exon 13 |
| | | | | | | | | Centroid Unpaired 1 |
| TI | 75 | 63* | uUCGAACAUAGGUAAUAGC | 36.8 | -21.37 | -28.92 | 7.44 | 5' UTR |
| | | | | | | | | Exon 1 |
| MM | 84 | 62* | uUGAGUCAGGGUUGCAAGG | 52.6* | -30.36** | -36.1* | 5.59 | Exon 11 |
| TQ | 12 | 61* | GAUGUUCGCUGGCUUUCCC | 57.9* | -35.06** | -38.51* | 3.08 | Exon 9 |
| CQ | 35 | 61* | CUCGAUGGUUGUUGACUCC | 52.6* | -26.97* | -35.12* | 6.48 | Exon 7 |
| CY | 43 | 59* | UUCGGCUCCUGGGUUCGGG | 68.4** | -28.2** | -41.9** | 10.6** | 5' UTR |
| | | | | | | | | Exon 1 |
| G3 | 8 | 59* | UUAGUUCACGAACCAUUCC | 42.1 | -25.28* | -30.62 | 5.17 | Exon 10 |
| | | | | | | | | Centroid Loop 6 |
| MQ | 73 | 59* | AUGAUAAGUGUGAAAAACC | 31.6 | -20.68 | -26.63 | 5.8 | Exon 12 |
| XT | 50 | 57* | AGCCUCGUGGAAACCAGGG | 63.2** | -22.87 | -40.2** | 13.29** | Exon 16 |
| | | | | | | | | 3' end of CDS |
| YB | 67 | 56* | uAUCCGGGAGAAAUCCAGC | 52.6* | -22.64 | -32.82 | 7.41 | Exon 3 |
| MI | 90 | 55* | AGGUAAUAGCCAGUGGAGC | 52.6* | -28.09** | -36.82* | 6.47 | Exon 3 |
| CM | 31 | 54* | CAGCGGGUCUGUAUGUGGG | 63.2** | -30.86** | -40.5** | 8.84* | 3' UTR |
| | | | | | | | | Exon 16 |
| TE | 25 | 52* | CUAGCGUUCGGCUCCUGGG | 68.4** | -28.19** | -42** | 11.18** | 3' UTR |
| | | | | | | | | Exon 16 |
| G2 | 6 | 52* | AUCGGGUUUCGGAGGUGCC | 63.2** | -32.31** | -39.1** | 5.5 | Exon 6 |
| | | | | | | | | Centroid Loop 3 |
| G4 | 9 | 52* | AAUAACUGGGGCCUGUGGC | 57.9* | -31.54** | -37.8* | 4.83 | Exon 16 |
| | | | | | | | | Centroid Loop 8 |
| MN | 79 | 51* | GAUAUGGAGAACCCAUGGG | 52.6* | -26.6* | -37.5* | 7.33 | Exon 12 |
| XS | 49 | 51* | GACGACAGUUUGUGCUUGG | 52.6* | -27.79* | -35.41* | 6.21 | Exon 16 |
| | | | | | | | | 3' end of CDS |
| Y7 | 62 | 50* | AGAUAUCAGGGGAGAGAGG | 52.6* | -33.03** | -37.5* | 4.26 | 5' UTR |
| | | | | | | | | Exon 1 |
| D2 | 47 | 50* | AGCGCCAGGGUUUAUGUCG | 57.9* | -26.66* | -35.8* | 8.46* | 3' UTR |
| | | | | | | | | Exon 16 |
| TF | 24 | 49* | uUAGCGUUCGGCUCCUGGG | 63.2** | -26.17* | -39.7** | 11.18** | 5' UTR |
| | | | | | | | | Exon 1 |
| CZ | 44 | 47* | UAAAGCGGGUCCCGAAGCG | 63.2** | -28.97** | -37.5* | 6.72 | Exon 7 |
| GJ | 27 | 45* | AACGUUGGUGGUGCACACG | 57.9* | -25.23* | -34.6 | 6.5 | Exon 1 |
| | | | | | | | | Signal peptide |
| MO | 78 | 43* | uGAUAUGGAGAACCCAUGG | 47.4 | -23.5 | -34.4 | 7.32 | Exon 12 |
| CV | 40 | 43* | AAACGAAUGAGACCAGUGC | 47.4 | -24.81* | -33.51 | 8.57* | 5' UTR |
| | | | | | | | | Exon 1 |
| CU | 39 | 42* | AGACGCCUGGGUUUGUACC | 57.9* | -25.59* | -37.22* | 9.84* | 5' UTR |
| | | | | | | | | Exon 1 |
| Y6 | 61 | 41* | GGCUAGUAACAUCAUCACC | 47.4 | -28.14** | -34.5 | 6.33 | 5' UTR |
| | | | | | | | | Exon 1 |
| Y1 | 56 | 40* | UGCAAUCGUUCCAUCGACC | 52.6* | -25.73* | -35.32* | 8.25* | 3' UTR |
| | | | | | | | | Exon 16 |
| MR | 72 | 39* | AAACCACCAAAUGCCUCCC | 52.6* | -22.95 | -35.32* | 12.37** | Exon 3 |
| Y2 | 57 | 39* | uUGAAUCGGGUUUCGGAGG | 52.6* | -26.46* | -34.7 | 6.37 | Exon 11 |
| CN | 32 | 34* | uACGCACUACCAUUCAUGC | 47.4 | -16.4 | -32.8 | 16.16** | Exon 14 |
| CR | 36 | 33* | AGCGGGUCUGUAUGUGGGG | 63.2** | -30.76** | -40.4** | 8.96* | Exon 7 |
| MV | 81 | 31** | CCCAGAGCAUUGCAGAUGG | 57.9* | -24.69* | -38.5* | 12.07** | Exon 11 |
| G7 | 19 | 31** | CAUCCUCAAUUCUUCUACC | 42.1 | -26.33* | -31.62 | 5.29 | Exon 16 |
| D1 | 46 | 29** | CACGGCACCCACUUCCCCG | 73.7** | -24.74* | -42.7** | 17.19** | 3' UTR |
| | | | | | | | | Exon 16 |
| D0 | 45 | 28** | uACGGCACCCACUUCCCCG | 68.4** | -23.65 | -40.5** | 16.09** | 5' UTR |
| | | | | | | | | Exon 1 |
| D3 | 48 | 28** | GUCUCCGCUUCCCAAACCC | 63.2** | -22.74 | -40.02** | 17.24** | Exon 15 |
| MJ | 89 | 26** | GAGCAACUGCAAGGUCAGC | 57.9* | -28.57** | -38.2* | 8.19* | Exon 6 |
| MK | 86 | 26** | UGAUGGAGCUUUGAUGAGC | 47.4 | -22.88 | -34.81 | 9.56* | Exon 11 |
| TS | 10 | 26** | UUAGUGACACUUUUAUUGG | 31.6 | -24.84* | -27.71 | 1.97 | 3'UTR |
| | | | | | | | | Centroid Loop 11 |
| | | | | | | | | Exon 16 |
| Y3 | 58 | 22** | CUGAAUCGGGUUUCGGAGG | 57.9* | -26.82* | -37.61* | 7.03 | 3'UTR |
| | | | | | | | | Exon 16 |
| | | | | | | | | Centroid loop 9 |
| MP | 76 | 14** | GACACCUGGUGCUUCCAGC | 63.2** | -26.12* | -41.6** | 11.36** | Exon 12 |
| TG | 23 | 14** | GGUUCGGGUAGAAAUGAGG | 52.6* | -24.65* | -35.41* | 9.72* | 5' UTR |
| | | | | | | | | Exon 1 |
| XZ | 54 | 12** | AGACACGAUCACGGCAUGG | 57.9* | -28.13** | -37.9* | 9.44* | Exon 3 |
| XW | 53 | 9** | CGACGCUGGCACUUCAGGG | 68.4** | -28.79** | -41.5** | 10.55** | Exon 6 |
| Y0 | 55 | 7** | UUCCCCGAUCUAGCGUUCG | 57.9* | -21.81 | -37.2* | 14.33** | Exon 4 |
| CK | 29 | 6** | CGGUGCGCCUGAAGACUGG | 68.4** | -31.67** | -40.8** | 7.01 | Exon 3 |
| MT | 98 | 4** | AAUGGACAAUGGAAUGGAA | 36.8* | -9.89 | -12.92 | 2.94 | Exon 10 |
| | | | | | | | | Centroid Loop 6 |
| TC | 28 | 3** | uGGUGCGCCUGAAGACUGG | 63.2** | -31.78** | -39.3** | 6.63 | Exon 3 |
| CT | 38 | 2** | AAAGCGGGUCCCGAAGCGC | 68.4** | -28.56** | -40.62** | 9.87* | 3' UTR |
| | | | | | | | | Exon 16 |
| TD | 26 | 0** | GUUCGGCUCCUGGGUUCGG | 68.4** | -29.76** | -42.3** | 10.6** | Exon 1 |
| | | | | | | | | Signal peptide |
| TV | 2 | 0** | AGGCCCGAAGAUGGCAGCC | 68.4** | -30.02** | -41.92** | 7.99* | Exon 3 |
| Y4 | 59 | 0** | AAGGCCCGAAGAUGGCAGC | 63.2** | -30.44** | -40.02** | 6.53 | 5' UTR |
| | | | | | | | | Exon 1 |
| XV | 52 | 0** | uGACGCUGGCACUUCAGGG | 63.2** | -27.2* | -39.3** | 9.94* | 5' UTR |
| | | | | | | | | Exon 1 |
| CS | 37 | 0** | UUGAACGGCCACAGACACC | 57.9* | -26.83* | -37.12* | 10.11** | Exon 11 |
| CO | 33 | 0** | CACGCACUACCAUUCAUGC | 52.6* | -17.25 | -35.2* | 17.71** | Exon 14 |
| MS | 99 | 0** | AUGGAAUGGAAUGGUUCGU | 42.1 | -10.58 | -14.6 | 1.9 | Exon 10 |
| | | | | | | | | Centroid Loop 6 |

TABLE 11
Thermodynamic property calculations for 21 bp mature miRNA guides

| | | | | Total free | Energy from | | |
|---|---|---|---|---|---|---|---|
| | % KD for | | | energy of | duplex | Opening | |
| | the corre- | | | binding | formation | energy | |
| | sponding | | | (kcal/mol) | (kcal/mol) | GluK2 | |
| siRNA | 21 bp | Guide | %GC | (21 bp | (21 bp | mRNA | Grik2 |
| ID | guide | sequence (21 base) | (21 bp) | guide) | guide) | (kcal/mol) | Target Region |
| MD | 0** | CAUUCCCAUAGCUAAUGCCUG | 47.6 | -28.72* | -39.04* | 9.13* | Exon 3 |
| G5 | 89 | UGAUAUUCCAUCUCCUGGCAA | 42.9 | -31.31** | -37.7 | 5.41 | 3'UTR |
| | | | | | | | Centroid |
| | | | | | | | Unpaired 3 |
| | | | | | | | Exon 16 |
| Y9 | 87 | CAUAGGUAAUAGCCAGUGGAG | 47.6 | -14.06 | -23.76 | 8.7 | Exon 5 |
| TO | 86 | CGUUCCACUGUCAGAAAGGCG | 57.1** | -31.37** | -39.2* | 6.15 | Exon 13 |
| | | | | | | | Centroid |
| | | | | | | | Unpaired 1 |
| TL | 85 | UGUCGAUUACACUGCAAGGAA | 42.9 | -29.31* | -36.31 | 6.72 | Exon 7 |
| TK | 85 | AGUCGAUGACCUUCUCUCGAA | 47.6 | -26.58 | -37.9 | 8.25* | 5' UTR |
| | | | | | | | Exon 1 |
| G0 | 83 | AAGGGUAGUAUUGGGUAGCAA | 42.9 | -35.17** | -37.31 | 1.74 | Exon 2 |
| | | | | | | | Centroid Loop 1 |
| TT | 83 | UGUUAACACCACUGUAUCGGU | 42.9 | -30.19* | -36.31 | 4.49 | Exon 6 |
| | | | | | | | Centroid Loop 2 |
| GI | 83 | GUCUCGAUAUGGAGAACCCAU | 47.6 | -28.24* | -40* | 7.3 | overlaps Exon 3 |
| | | | | | | | and Exon 4 |
| MU | 82 | UUCAUAUGUAAAGCCAAGGAU | 33.3 | -28.52* | -32.31 | 3.67 | Exon 10 |
| | | | | | | | Centroid Loop 5 |
| TP | 82 | uGUUCCACUGUCAGAAAGGCG | 52.4* | -31.61** | -39.4* | 6.15 | Exon 13 |
| | | | | | | | Centroid |
| | | | | | | | Unpaired 1 |
| Y5 | 81 | UUGAGUCGAAGAUUAUACCUU | 33.3 | -26.28 | -32.51 | 5.81 | 5' UTR |
| | | | | | | | Exon 1 |
| CW | 80 | AUAAACGCAGUCCACUUCCAA | 42.9 | -26.87 | -35.41 | 8.52* | Exon 7 |
| Y8 | 80 | uAUAGGUAAUAGCCAGUGGAG | 42.9 | -27.01* | -36.01 | 8.7* | Overlaps Exon 4 |
| | | | | | | | and Exon 5 |
| ML | 80 | AGAGCUCCAACUCCAAACCAG | 52.4* | -29.07* | -40.01* | 9.64** | Exon 11 |
| TJ | 80 | GAUGCGUCGAGUGGUGACCGC | 66.7** | -31.07** | -45.22** | 10.61** | Exon 11 |
| MW | 79 | CAGAGCAUUGCAGAUGGACUG | 52.4* | -25.99 | -39* | 9.82** | Exon 4 |
| TM | 78 | UAUUUCCUUGAUAAUUGGCAU | 28.6 | -25.71 | -30.71 | 4.58 | 3' UTR |
| G9 | 77 | CCCAUAGCUAAUGCCUGUUUU | 42.9 | -29.4* | -36.61 | 5.54 | Overlaps with |
| | | | | | | | Exon 14 and 15 |
| ME | 77 | uUGUCAUCAUUCCCAUAGCUA | 38.1 | -24.39 | -34.7 | 10.25** | Exon 14 |
| G6 | 76 | uAUCCUCAAUUCUUCUACCAU | 33.3 | -25.34 | -25.34 | 5.27 | Exon 16 |
| GE | 76 | UUAGUCGGAGAGCAUCCGGGA | 57.1** | -31.16** | -42.3** | 5.42 | Exon 13 |
| | | | | | | | Centroid |
| | | | | | | | Unpaired 1 |
| MH | 74 | uGUCAUAAAUCCAUCCAGCAA | 38.1 | -26.41 | -34.41 | 7.98 | Exon 6 |
| TN | 73 | AUAAGUGAUGAACAUCUGCUU | 33.3 | -26.75 | -32.31 | 3.94 | 3'UTR |
| | | | | | | | Centroid |
| | | | | | | | Unpaired 4 |
| | | | | | | | Exon 16 |
| GD | 72 | UGUAGAUAACUCUCUGAGGAG | 42.9 | -29.58* | -37.4 | 5.74 | Exon 10 |
| | | | | | | | Centroid Loop 5 |
| TU | 72 | AGCAUUGCAGAUGGACUGCAC | 52.4* | -24.17 | -40.4* | 10.78** | Exon 3 |
| TR | 71 | UUAUUAUUUGGGAUAUGGGGG | 38.1 | -33.64** | -34.81 | 0.89 | 3'UTR |
| | | | | | | | Centroid Loop 12 |
| | | | | | | | Exon 16 |
| MG | 71 | uAUCAGUCGUCAUAAAUCCAU | 33.3 | -25.01 | -30.71 | 5.69 | Exon 6 |
| TH | 70 | CUCGAACAUAGGUAAUAGCCA | 42.9 | -25.56 | -36.61 | 8.79* | 5' UTR |
| | | | | | | | Exon 1 |
| MF | 69 | UAGAGACUGACAUAGAAGGAA | 38.1 | -26.72 | -35.6 | 8.52* | Exon 3 |
| CL | 69 | uAGCGGGUCUGUAUGUGGGGA | 57.1** | -32.51** | -42.4** | 9.01* | 3' UTR |
| | | | | | | | Exon 16 |
| XU | 68 | UGUCUCGAUAUGGAGAACCCA | 47.6 | -27.87* | -39.6* | 7.27 | 5' UTR |
| | | | | | | | Exon 1 |
| YA | 67 | GGUUGCAUAUUUCCACAGGAA | 42.9 | -28.12* | -36.81 | 7.2 | Overlaps Exon 4 |
| | | | | | | | and Exon 5 |
| GG | 67 | AGUCGUCAUAAAUCCAUCCAG | 42.9 | -28.84* | -36.21 | 7.36 | Exon 11 |
| XX | 67 | uACCACGUCUGAGUCAGGGUU | 52.4* | -28.99* | -39.4* | 8.47* | Exon 6 |
| XY | 67 | CACCACGUCUGAGUCAGGGUU | 57.1** | -30.66** | -43.1** | 10.36** | Exon 6 |
| CP | 66* | uUCGAUGGUUGUUGACUCCAU | 42.9 | -27.3* | -35.7 | 6.32 | Exon 3 |
| G8 | 66* | ACUGACAUAGAAGGAAUCUUU | 33.3 | -23.31 | -32.31 | 8.22* | Overlaps with |
| | | | | | | | Exon 14 and 15 |
| CX | 65* | GACCGCAGACACGAUCACGGC | 66.7** | -29.57* | -46.12** | 14.57** | 5' UTR |
| | | | | | | | Exon 1 |
| G1 | 64* | AAUCGGGUUUCGGAGGUGCCU | 57.1** | -32.35** | -41.4** | 6.19 | Exon 6 |
| | | | | | | | Centroid Loop 3 |
| TI | 63* | uUCGAACAUAGGUAAUAGCCA | 38.1 | -23.18 | -33.71 | 8.26 | 5' UTR |
| | | | | | | | Exon 1 |
| GF | 63* | UGCGUCGAGUGGUGACCGCAG | 66.7** | -28.78* | -45.3** | 10.72** | Exon 13 |
| | | | | | | | Centroid |
| | | | | | | | Unpaired 1 |
| MM | 62* | uUGAGUCAGGGUUGCAAGGGU | 52.4* | -34.81** | -40.5* | 5.56 | Exon 11 |
| TQ | 61* | GAUGUUCGCUGGCUUUCCCUU | 52.4* | -37.53** | -41.11** | 3.08 | Exon 9 |
| CQ | 61* | CUCGAUGGUUGUUGACUCCAU | 47.6 | -29.47* | -38.1* | 6.49 | Exon 7 |
| G3 | 59* | UUAGUUCACGAACCAUUCCAU | 38.1 | -28.19* | -33.61 | 5.23 | Exon 10 |
| | | | | | | | Centroid Loop 6 |
| MQ | 59* | AUGAUAAGUGUGAAAAACCAC | 33.3 | -23.62 | -30.93 | 6.35 | Exon 12 |
| CY | 59* | UUCGGCUCCUGGGUUCGGGUA | 61.9** | -30.21* | -44.5** | 10.62** | 5' UTR |
| | | | | | | | Exon 1 |
| XT | 57* | AGCCUCGUGGAAACCAGGGGU | 61.9** | -24.03 | -45.2** | 13.35** | Exon 16 |
| | | | | | | | 3' end of CDS |
| YB | 56* | uAUCCGGGAGAAAUCCAGCAC | 52.4* | -26.46 | -39.6* | 10.38** | Exon 3 |
| M1 | 55* | AGGUAAUAGCCAGUGGAGCAA | 47.6 | -29.53* | -38.3* | 6.47 | Exon 3 |
| CM | 54* | CAGCGGGUCUGUAUGUGGGGA | 61.9** | -35.53** | -45.6** | 9.07* | 3' UTR |
| | | | | | | | Exon 16 |
| G2 | 52* | AUCGGGUUUCGGAGGUGCCUG | 61.9** | -32.87** | -41.7** | 4.35 | Exon 6 |
| | | | | | | | Centroid Loop 3 |
| G4 | 52* | AAUAACUGGGGCCUGUGGCUU | 52.4* | -31.27** | -41.2** | 5.75 | Exon 16 |
| | | | | | | | Centroid Loop 8 |
| TE | 52* | CUAGCGUUCGGCUCCUGGGUU | 61.9** | -30.05* | -43.9** | 11.18** | 3' UTR |
| | | | | | | | Exon 16 |
| XS | 51* | GACGACAGUUUGUGCUUGGGU | 52.4* | -32.54** | -40.41* | 6.29 | Exon 16 |
| | | | | | | | 3' end of CDS |
| MN | 51* | GAUAUGGAGAACCCAUGGGAG | 52.4* | -29.9* | -42.4** | 8.92* | Exon 12 |
| Y7 | 50* | AGAUAUCAGGGGAGAGAGGAU | 47.6 | -36.18** | -40.7* | 4.28 | 5' UTR |
| | | | | | | | Exon 1 |
| D2 | 50* | AGCGCCAGGGUUUAUGUCGAU | 52.4* | -29.53* | -39.01* | 8.47* | 3' UTR |
| | | | | | | | Exon 16 |
| TF | 49* | uUAGCGUUCGGCUCCUGGGUU | 57.1** | -27.5* | -41* | 11.11** | 5' UTR |
| | | | | | | | Exon 1 |
| CZ | 47* | UAAAGCGGGUCCCGAAGCGCC | 66.7** | -32.36** | -45.12** | 10.05** | Exon 7 |
| GJ | 45* | AACGUUGGUGGUGCACACGCA | 57.1** | -25.32 | -40.4* | 11.59** | Exon 1 |
| | | | | | | | Signal peptide |
| MO | 43* | uGAUAUGGAGAACCCAUGGGA | 47.6 | -28* | -38.9* | 7.33 | Exon 12 |
| CV | 43* | AAACGAAUGAGACCAGUGCUG | 47.6 | -29.23* | -38.01* | 8.61* | 5' UTR |
| | | | | | | | Exon 1 |
| CU | 42* | AGACGCCUGGGUUUGUACCAU | 52.4* | -27.53* | -40.2* | 9.85** | 5' UTR |
| | | | | | | | Exon 1 |
| Y6 | 41* | GGCUAGUAACAUCAUCACCUC | 47.6 | -30.87** | -39* | 8.03* | 5' UTR |
| | | | | | | | Exon 1 |
| Y1 | 40* | UGCAAUCGUUCCAUCGACCAC | 52.4* | -30.09* | -39.8* | 8.27* | 3' UTR |
| | | | | | | | Exon 16 |
| Y2 | 39* | uUGAAUCGGGUUUCGGAGGUG | 52.4* | -30.59** | -38.51 | 6.45 | Exon 11 |
| MR | 39* | AAACCACCAAAUGCCUCCCAC | 52.4* | -24.42 | -36.81 | 12.37** | Exon 3 |
| CN | 34* | uACGCACUACCAUUCAUGCUU | 42.9 | -18.32 | -35.3 | 16.16** | Exon 14 |
| CR | 33* | AGCGGGUCUGUAUGUGGGGAA | 57.1** | -33.16** | -43.2** | 9.01* | Exon 7 |
| G7 | 31** | CAUCCUCAAUUCUUCUACCAU | 38.1 | -27.81* | -33.11 | 5.29 | Exon 16 |
| MV | 31** | CCCAGAGCAUUGCAGAUGGAC | 57.1** | -27.12* | -43.2** | 13.26** | Exon 11 |
| D1 | 29** | CACGGCACCCACUUCCCCGAU | 66.7** | -27.54* | -46** | 17.33** | 3' UTR |
| | | | | | | | Exon 16 |
| DO | 28** | uACGGCACCCACUUCCCCGAU | 61.9** | -26.46 | -43.7** | 16.11** | 5' UTR |
| | | | | | | | Exon 1 |
| D3 | 28** | GUCUCCGCUUCCCAAACCCAU | 57.1** | -25.72 | -43** | 17.25** | Exon 15 |
| TS | 26** | UUAGUGACACUUUUAUUGGUU | 28.6 | -26.47 | -29.61 | 1.97 | 3'UTR Centroid |
| | | | | | | | Loop 11 |
| | | | | | | | Exon 16 |
| MJ | 26** | GAGCAACUGCAAGGUCAGCUU | 52.4* | -29.33* | -40.8* | 8.2* | Exon 6 |
| MK | 26** | UGAUGGAGCUUUGAUGAGCUC | 47.6 | -22.76 | -39.31* | 9.87** | Exon 11 |
| Y3 | 22** | CUGAAUCGGGUUUCGGAGGUG | 57.1** | -30.57** | -41.61** | 7.28 | 3'UTR |
| | | | | | | | Exon 16 |
| | | | | | | | Centroid Loop 9 |
| GH | 21** | AUGCGUCGAGUGGUGACCGCA | 61.9** | -27.23* | -43.3** | 10.61** | Exon 11 |
| TG | 14** | GGUUCGGGUAGAAAUGAGGAU | 47.6 | -27.7* | -38.61* | 9.73** | 5' UTR |
| | | | | | | | Exon 1 |
| MP | 14** | GACACCUGGUGCUUCCAGCGG | 66.7** | -31.64** | -47.3** | 11.46** | Exon 12 |
| XZ | 12** | AGACACGAUCACGGCAUGGUC | 57.1** | -30.15* | -42.02** | 9.58** | Exon 3 |
| XW | 9** | CGACGCUGGCACUUCAGGGAC | 66.7** | -32.76** | -46.02** | 11.1** | Exon 6 |
| Y0 | 7** | UUCCCCGAUCUAGCGUUCGGC | 61.9** | -21.79 | -43.8** | 17.49** | Exon 4 |
| CK | 6** | CGGUGCGCCUGAAGACUGGAU | 61.9** | -34.73** | -44** | 7.04 | Exon 3 |
| MT | 4** | AAUGGACAAUGGAAUGGAAUG | 38.1 | -27.94* | -34.81 | 4.66 | Exon 10 |
| | | | | | | | Centroid Loop 6 |
| TC | 3** | uGGUGCGCCUGAAGACUGGAU | 57.1** | -34.65** | -42.7** | 6.65 | Exon 3 |
| CT | 2** | AAAGCGGGUCCCGAAGCGCCA | 66.7** | -32.68** | -45.6** | 10.01** | 3' UTR |
| | | | | | | | Exon 16 |
| MS | 0** | AUGGAAUGGAAUGGUUCGUGA | 42.8 | -30.67** | -36.71 | 1.92 | Exon 10 |
| | | | | | | | Centroid Loop 6 |
| Y4 | 0** | AAGGCCCGAAGAUGGCAGCCA | 61.9** | -31.24** | -44.3** | 7.99 | 5' UTR |
| | | | | | | | Exon 1 |
| TV | 0** | AGGCCCGAAGAUGGCAGCCAC | 66.7** | -32.37** | -46.22** | 8.78* | Exon 3 |
| XV | 0** | uGACGCUGGCACUUCAGGGAC | 61.9** | -31.7** | -44.02** | 10.16** | 5' UTR |
| | | | | | | | Exon 1 |
| CS | 0** | UUGAACGGCCACAGACACCAC | 57.1** | -30.92** | -41.42** | 10.17** | Exon 11 |
| TD | 0** | GUUCGGCUCCUGGGUUCGGGU | 66.7** | -32.95** | -47.2** | 10.62** | Exon 1 |
| | | | | | | | Signal peptide |
| CO | 0** | CACGCACUACCAUUCAUGCUU | 47.6 | -19.24 | -37.81 | 17.74** | Exon 14 |

Column definitions for Table and Table 11:

% KD: Percent knockdown of luciferase-GluK2 mRNA (Grik2 mRNA, variant 1, SEQ ID NO: 122) reporter expression achieved by co-transfection with candidate siRNAs (for the equivalent 19 bp RNA guides as determined by the assay described in Example 1A); cells with two asterisks (**)=less than 33% knockdown (KD), cells with 1 asterisk (*)=33-66% KD, and cells without asterisks=greater than 66% KD.

%GC: Combined percentage of bases in guide sequence of siRNA that are guanine and cytosine; Table 10: cells with two asterisks (**)=greater than 60% GC, cells with one asterisk (*)=50-60% GC, and cells without asterisks=less than 50% GC; Table 11: cells with two asterisks (**)=greater than 55% GC, cells with one asterisk (*)=50-55% GC, and cells without asterisks=less than 55% GC.

Grik2 target regions in bold letters signify an identified loop or unpaired region of the disclosure.

Total Free Energy of Binding (kcal/mol): free energy of the process of an siRNA hybridizing to its corresponding target sequence on the Grik2 mRNA (SEQ ID NO: 122), including opening the target region on the mRNA, generation of single-stranded guide, and hybridization of the single-stranded siRNA guide to its single-stranded mRNA target sequence; Table 10: cells with two asterisks (**)=less than −28 kcal/mol (greatest deltaG; most negative values), cells with one asterisk (*)=−23 to −24 kcal/mol, and cells with no asterisks =-24 kcal/mol (lowest deltaG; closest to zero); Table 11: cells with two asterisks (**)=less than −30.5 kcal/mol (greatest deltaG; most negative values), cells with one asterisk (*)=-30.5 to −27 kcal/mol, and cells with no asterisks=−27 kcal/mol (lowest deltaG; closest to zero).

Energy from Duplex Formation (kcal/mol): free energy of hybridization of single-stranded siRNA guide to single-stranded mRNA target sequence; Table 10: cells with two asterisks (**)=less than −38 kcal/mol (greatest deltaG; most negative values), cells with one asterisk (*)=-38 to -kcal/mol, and cells without asterisks=-kcal/mol (lowest deltaG; closest to zero); Table 11: cells with two asterisks (**)=less than −41 kcal/mol (greatest deltaG; most negative values), cells with one asterisk (*)cells=-41 to −38 kcal/mol, and cells without asterisks =-38 kcal/mol (lowest deltaG; closest to zero).

Opening Energy Gluk2 mRNA (kcal/mol) (also known as Target Opening Energy): energy required to resolve RNA secondary structure at the target location (may include resolution of nearby secondary structures or involvement of distal sequences that form secondary structure with target sequence); Table 10: cells with two asterisks (**)=highest energy requirement (>kcal/mol =most positive/least favorable), cells with one asterisk (*)=moderate energy requirement (between 7.5 and kcal/mol; moderately favorable), cells without asterisks=lowest energy requirement (<7.5 kcal/mol; closest to zero/most favorable); Table 11: cells with two asterisk (**)=highest energy requirement (>9.5 kcal/mol =most positive/least favorable), cells with one asterisk (*)=moderate energy requirement (between 8 and 9.5 kcal/mol; moderately favorable), cells without asterisks =lowest energy requirement (<8 kcal/mol; closest to zero/most favorable).

To determine Opening Energy of GIuK2 mRNA, Energy from Duplex Formation, and Total Free Energy of binding, RNAup WebServer (rna.tbi.univie.ac.at//cgi-bin/RNAWebSuite/RNAup.cgi, part of the ViennaRNA Web Services software suite) was used (Lorenz, R., Bernhart, S.H., HOner zu Siederdissen, C., Tafer, H., Flamm, C., Stadler, P.F., and Hofacker, Ivo L. ViennaRNA Package 2.0, Algorithms for Molecular Biology, 6:1 26, 2011, doi:10.1186/1748-7188-6-26). RNAup folds each of two input RNAs (as performed by the RNAfold server, rna.tbi.univie.ac.at/cgi-bin/RNAWebSuite/RNAfold.cgi) and then, based on the thermodynamics of each of the folded RNAs, calculates various thermodynamic values relating to the hybridization of the input RNAs. These include the energy (in kcal/mol) required to open secondary structures on either RNA (e.g., target RNA and siRNA), the energy generated from duplex formation (hybridization of one unfolded RNA to the other, unfolded RNA), as well as the total energy of binding, which takes into account both the energy required to open each RNA as well as the energy of the hybridization itself.

Here, RNAup was used to calculate the thermodynamic parameters of siRNA guide sequences binding to the human Grik2 mRNA (SEQ ID NO: 122) in order to determine thresholds that may be predictive of siRNA activity (e.g., efficacy of knockdown) in an experimental setting, either in vitro or in vivo. Results are listed in Table for 19 bp synthetic RNA guides, and Table 11 for 21 bp mature miRNA guides. For the purposes of this example, the 19 bases of the 21 base siRNA sequence that are homologous/complementary to the Grik2 mRNA sequence (those siRNAs that were tested in the in vitro luciferase reporter assay as described in Example 1A) were queried against the full length human Grik2 mRNA. While this approach was taken with respect to 19 bases of the siRNA sequence, it can also be used to query longer ASOs, shRNAs, miRNAs, or shmiRNAs as it was done for the 21 bp mature miRNAs, since the principles of folding/unfolding and hybridization are generally considered to be comparable. The above-described thermodynamic parameters were recorded (see Tables and 11) and evaluated for their correlation with % knockdown (KD) in the luciferase reporter assay and, therefore, their ability to predict the efficacy of a given guide sequence.

### Target Opening Energy

When candidate guide sequences were ranked according to the energy predicted to be required to open their corresponding target sequence in the GluK2 mRNA, there is an apparent correlation between guides targeting regions with low opening energy requirements (closest to 0 kcal/mol) and those guides determined to have a greater capacity for reporter knockdown. Target opening energies ranged from 0.88 to 17.71 kcal/mol with a mean of 8.12 kcal/mol and a median of 7.41 kcal/mol; siRNAs that knocked down reporter expression by >66% trended towards having a lower target opening energy (mean=7.12 kcal/mol, median=6.66 kcal/mol), while those siRNAs that did not knockdown reporter expression as efficiently had a higher target opening energy (mean=8.74 kcal/mol, median=8.34 kcal/mol). While there were outliers in both groups (e.g., GO: %KD=83 while opening energy=17.24 kcal/mol; and MT: %KD=4 while opening energy=1.9 kcal/mol), there was an apparent enrichment of targets with lower opening energies amongst the siRNA guides that knocked down reporter expression at levels greater than 66%. Mechanistically, it follows that target sequences that require less energy input to unfold are more amenable to unfolding and can therefore be considered “more accessible” for siRNA binding. Furthermore, there is an enrichment for predicted secondary Grik2 loop and unpaired structures, as shown in FIG. 1B and Table 4. For example, an RNA guide having a length of no more than 23 nucleotides that specifically hybridizes within a single-stranded region of a Grik2 mRNA exhibits a favorable Opening energy of at least <kcal/mol, and in certain instances <7.5 kcal/mol. An enriched cluster of siRNAs, including those guides disclosed herein that bind to predicted secondary Grik2 loop and unpaired structures as shown in FIG. 1B, have a Target Opening Energy of less than kcal/mol and correlated with greater than 66% Gluk2 knockdown. The 19 bp Grik2-targeting guides that have Target Opening Energy predictive values of <7.5 kcal/mol are more likely to be efficacious; whereas Target Opening Energy predictive values of >kcal/mol are in some cases less favorable. The 21 bp Grik2-targeting guides that have Target Opening Energy predictive values of <8.0 kcal/mol are more likely to be efficacious; whereas Target Opening Energy predictive values of >9.5 kcal/mol are in some embodiments less favorable (FIGS. 1J and 1K).

### Energy of Duplex Formation

The same Grik2 mRNA/siRNA guide sequence pairs were then queried to determine the predicted energy of duplex formation. There was an apparent correlation between guides with higher reporter knockdown and those with higher energies of duplex formation (closer to zero). Energies of duplex formation ranged from −42.7 to −12.92 kcal/mol with a mean of 35.28 kcal/mol and a median of −35.41 kcal/mol. Guide siRNA sequences with a >66% KD had, on average, higher energies of duplex formation (mean=−33.5 kcal/mol, median=−33.3 kcal/mol) while those with <66% KD had, on average, lower energies of duplex formation (mean=−36.4 kcal/mol, median=−37.5 kcal/mol). More negative/lower energy of duplex formation indicates that the formation of a given duplex is more favorable than the formation of a duplex for which the energy of duplex formation is higher/closer to zero. Therefore, there is an inverse correlation between favorability of duplex formation and efficiency of siRNA guide sequence knockdown efficiency, which may seem counterintuitive. However, this suggests that energy of duplex formation is more critical when determining the favorability of duplex separation (its inverse) rather than duplex formation. In this context, we refer to this value as a measure of duplex stability; the lower the value, the more stable the duplex. If the target:siRNA duplex is less stable, then the siRNA may be more efficient from a knockdown perspective due to its increased processivity. That is to say, it is more likely to disengage from less stable duplexes in order to target the same region on a different mRNA molecule, which directly relates to its efficiency. Conversely, if the target:siRNA duplex is too stable, then the siRNA (bound to RISC) is less likely to disengage from its target.

The 19 bp Grik2-targeting guides that have Energy of Duplex Formation predictive values of >kcal/mol are more likely to be efficacious; whereas Energy of Duplex Formation predictive values of >−39 kcal/mol are in some embodiments less favorable. The 21 bp Grik2-targeting guides that have Energy of Duplex Formation predictive values of >−38 kcal/mol are more likely to be efficacious; whereas Energy of Duplex Formation predictive values of >−41 kcal/mol are in some embodiments less favorable (FIGS. 1L and 1M).

### Total Energy of Binding

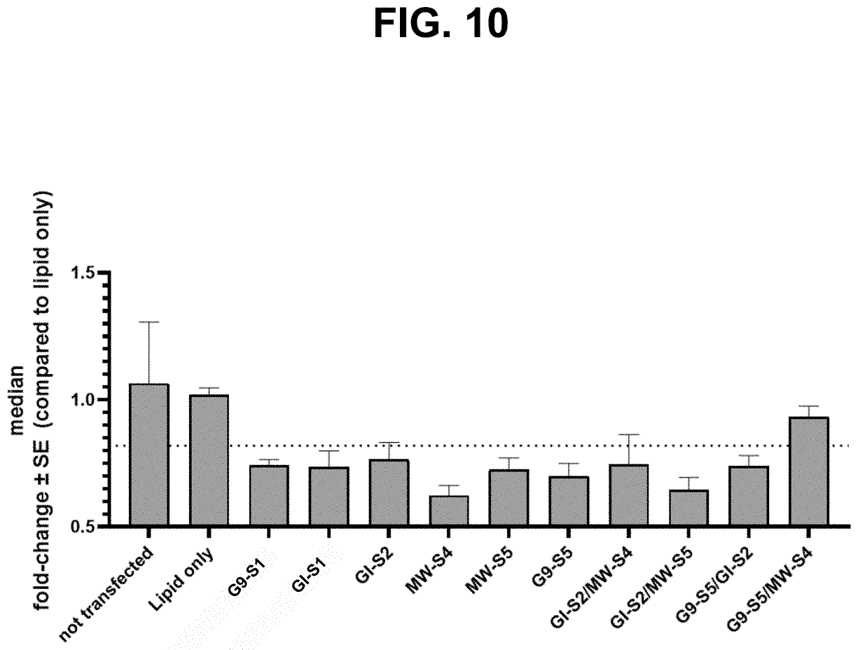

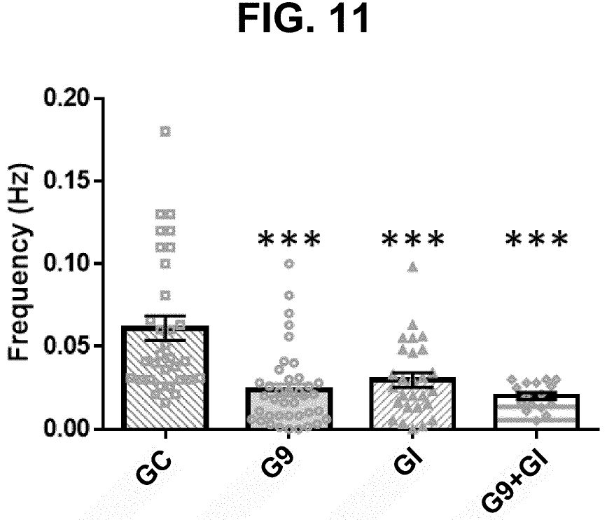

Finally, the Total Energy of Binding was determined for each siRNA/miRNA guide sequence to the target GluK2 mRNA. Total energy of binding ranged from −35.06 to −9.89 kcal/mol, with a mean of −25.93 kcal/mol and a median of −26.29 kcal/mol for 19 bp siRNA guides. While the mean values for total energy of binding for the siRNAs with >66% KD or <66% were not markedly different (˜25.65 vs -26.25 kcal/mol), the difference in median values was larger (˜25.35 vs. -26.66 kcal/mol, respectively), with almost no siRNA guides with >66% KD having a total energy of binding less than −kcal/mol (1 of 37, as compared to <66% KD guides, where 13 of 61 guides had a total energy of binding less than −kcal/mol). Therefore, this suggests that a Total Energy of Binding less than −kcal/mol in some cases indicates that a given guide sequence is more likely to efficiently knockdown a target mRNA. The 19 bp Grik2-targeting guides that have Total Energy of Binding predictive values of >-24 kcal/mol are more likely to be efficacious; whereas Total Energy of Binding predictive values of >−28 kcal/mol are in some embodiments less favorable. The 21 bp Grik2-targeting guides that have Total Energy of Binding predictive values of >−27 kcal/mol are more likely to be efficacious; whereas Total Energy of Binding predictive values of >−30.5 kcal/mol are in some embodiments less favorable (FIGS. 101N and 10).

Guanine-Cytosine (GC) content (% GC)

Finally, the GC content of the siRNA or miRNA guide sequences was determined. The group of siRNA guides causing a >66% KD in the reporter assay had, on average, lower GC content (mean=46.23%, median=47.4%) than the remaining guides with <66% KD (mean=56.4%, median=57.9%). Therefore, a low GC content is a strong predictor of guide efficiency, and, conversely, a high GC content is a contraindicator for guide efficiency. The factors contributing to this correlation are likely complex and relate to a number of thermodynamic parameters. First, low GC content of the guide sequence also indicates that the passenger sequence of the siRNA duplex (or, precursor shRNA or miRNA stem) contains a low percentage of GCs; therefore, this duplex is more easily separated into guide and passenger strands, enabling the guide strand to “mature” due to an enrichment of “weaker” base pairing contributions from A:U pairings. The target sequence in the mRNA is related to the guide sequence that targets it; therefore, if a given guide has low GC content, the target sequence, even if it is not perfectly complementary, likely also has low GC content. If significant secondary structure is predicted at the site of that target sequence, it will require less energy to “open” in order to be accessed by the antisense RNA, which we have also determined to be a favorable parameter for siRNA targeting. Lower GC content in the siRNA:target duplex also results in a lower stability of the duplex (as measured by energy of duplex formation), again due to the lack of more stable G:C base pairing, allowing for disengagement of the antisense RNA to allow additional mRNA molecules to be targeted. Taken together, GC content of the siRNA directly relates to other thermodynamic parameters described here and may be inextricable from those correlations established above.

The 19 bp Grik2-targeting guides that have GC content values of <50% are more likely to be efficacious; whereas GC content values of >60% are in some embodiments less favorable. The 21 bp Grik2-targeting guides that have GC content values of <50% are more likely to be efficacious; whereas GC content values of >55% are in some embodiments less favorable (FIGS. 1P and 10).

### Example2

### Validation of Anti-Epileptic Effects of Grik2-Targeting Antisense Oligonucleotide Constructs in Murine, Rodent, and Human Model Systems

### Materials and Methods

For protocols using mice, experiments were approved by the Institut National de la Sante et de la Recherche Medicale (INSERM) animal care and use committee and authorized by the Ministere de I'Education Nationale, de l'Enseignement Superieur et de la Recherche in accordance with the European community council directives (2010/63/UE).

Murine Hippocampal Organotypic Slices

Organotypic hippocampal slice cultures (350 μm) were prepared from wild-type Swiss mice (P9-10) using a Mcllwain tissue chopper as previously described (Peret et al. 2014). Slices were placed on mesh inserts (Millipore) inside culture dishes containing 1 mL of the following medium: MEM 50%, HS 25%, HBSS 25%, HEPES mM, glucose 6.5 mg/mL and insulin 0.1 mg/mL. Culture medium was changed every two to three days and slices maintained in an incubator at 37° C./5% CO_{2.}

Electrophysiological Recordings

Mice organotypic slices were individually transferred to a recording chamber maintained at 30-32° C. and continuously perfused (2-3 mL/min) with oxygenated (95% 02 and 5% CO₂) ACSF (physiological condition), or ACSF containing a GABAA receptor antagonist, gabazine (5 μM; hyperexcitable condition), or ACSF containing 5 μM gabazine and 50 μM 4-AP (highly hyperexcitable condition). Local field potential (LFP) recordings were made with a monopolar Nichrome wire placed in the granule cell layer of the DG. A DAM-80 amplifier (lowpass filter: 0.1 Hz; highpass filter: 3 KHz; World Precision Instruments, Sarasota, FL) was used. Data were digitized (kHz) with a Digidata 1440A (Molecular Devices) to a computer, and acquired using Clampex 10.1 software (PClamp, Molecular Devices). Signals were analyzed offline using Clampfit 9.2 (PClamp) and MiniAnalysis 6.0.1 (Synaptosoft, Decatur, Ga.).

Design and production of viral vectors targeting Grik2 mRNA by RNAi RNAi (e.g., ASO) sequences were designed using the Smart selection design (Dharmacon)(Birmingham et. al., 2007). The efficiency of RNAi sequences either as shRNAs or as miRNAs using the miR-structure was compared. Lentiviral or AAV9 vectors encoding a human Grik2 antisense sequence (G9; SEQ ID NO: 68) under control of the CAG (SEQ ID NO: 737) promoter or the hSyn promoter were used.

Neuronal Cell Cultures

Gestant rat females were purchased from Janvier Labs (Saint-Berthevin, France), and wild-type mice were produced. Animals were handled and euthanized according to European ethical rules. Dissociated hippocampal neurons from E18 Sprague—Dawley rat embryos or dissociated cortical neurons from PO mice were prepared as described by Kaech S. & Banker G (Culturing hippocampal neurons. Nat. Protoc. 1, 2406-2415 (2006)). Neurons were seeded in six-well plates coated with 1 mg/mL polylysine for two hours at a concentration of 500,000 cells per well. Neurons were cultured in conditioned Neurobasal medium (rats) or Neurobasal-A medium (mice), supplemented with 2 mM L-glutamine and 1 NeuroCult SM1 Neuronal supplement (STEMCELL Technologies) and renewed every three to four days. Two to three days after plating, half of the medium was removed and viral constructs were added with MOI 75000 for four hours. After four hours of contact with the viral constructs in a reduced medium volume, new medium supplemented with Ara-C (3.4 mM) was added to prevent glial cells growing.

WesternBlotting

Ten days after infection, DIV 12-13 murine or rat neuronal cultures were rinsed in ice-cold PBS, and then scraped into 150 p.1_{—} lysis buffer (50 mM HEPES, 100 mM NaCl, 1% Glycerol, 0.5% n-Dodecyl β-D-maltoside, pH 7.2; anti-protease and anti-phosphatase). Homogenates were kept on a wheel for 2 hours at cold temperature and centrifuged at 8000g for min at 4° C. to remove cell debris. The total protein content was quantified by Pierce BCA protein assay kit (23225, ThermoScientific) in duplicate on pt per condition.

Ten pg of proteins per condition were loaded on SDS—PAGE gel for Western blot analysis. A pre-stained protein ladder (26619, ThermoScientific) was loaded to control the weight. Samples were separated on 4-15% gradient pre-cast gels (Bio-Rad) and then transferred to nitrocellulose membranes for immunoblotting analysis. After blocking with 5% bovine serum albumin (BSA; Sigma) in Tris-buffered saline Tween-(TBST; 28 mM Tris, 136.7 mM NaCl, 0.05% Tween-20, pH 7.4), samples were maintained for one hour at room temperature on a shaker platform. Membranes were cut in two parts according to the ladder at the level of the 72KDa marker. Each part was incubated overnight at 4° C. with the respective primary antibodies diluted in blocking reagent (37516, ThermoScientific; antibodies solution could be re-used 3 times). The membrane portion with heavy weight proteins (72-250 kDa) was incubated with rabbit anti-GluK2 antibody (04-921; Merck) diluted at 1:2000 and the membrane with lighter weight proteins (17-72 kDa) was incubated with murine anti-(3-actin antibody (A5316; Sigma). On the next day, the membranes were washed three times for minutes each with TBST. An appropriate HRP-conjugated secondary antibody produced in donkey (Jackson ImmunoResearch) was diluted 1:5000 in 5% BSA—TBST and incubated with the membrane for one hour at room temperature. The target proteins were detected by chemiluminescence with clarity Western ECL (170-5060, Bio-Rad) on the ChemiDoc Touch system (Bio-Rad). The theoretical molecular weight of GluK2 is 103 kDa. For quantification, the intensity of the chemiluminescence signal of each lane was normalized to the control condition and thereafter to the actin band.

Viral Transduction of Organotypic Slices

For transduction of murine slices, 1μL of PBS containing lentiviral or AAV constructs was dropped directly on slices at DIVO for murine slices. Viral titers were 8×10⁸ genome copies (GC)/mL for LV-Control, 7.9×10⁸ GC/mL for LV-G9-shRNA, 3.0×10⁹ GC/mL for LV-G9-miRNA, 2.8×10¹³ GC/mL for AAV9-G9-miRNA, and 9.0×10¹² for AAV9-GFP-GC.

Immunolabelling

For Prox1, and GFP immunostaining, slices were fixed, and then permeabilized (0.5% Triton) in blocking solution containing 5% normal goat serum (NGS) in 0.5% Triton for one hour at room temperature. Slices were then incubated with the polyclonal rabbit anti-Prox1 antibody (Millipore) at 1:2000 in 5% NGS, and with polyclonal chicken anti-EGFP (Abcam, Cambridge, UK; RRID: AB-300798) at 1:1000 in 0.5% Triton overnight at 4° C. Slices were incubated for two hours in secondary antibody Alexa488 (Invitrogen 1:500), then cover-slipped in Fluoromount (Thermo Fisher). Fluorescence images were acquired with a LSM800 Zeiss confocal microscope using 10X/0.3NA and 20X/0.8NA objectives. Images were processed using NIH ImageJ software.

Statistical Analysis

All values are given as means ±SEM otherwise specified. Statistical analyses were performed using Graphpad Prism 7 (GraphPad Software, La Jolla, CA). The Shapiro—Wilk test was used to determine normality of the data. The parametric Student's t-test (paired and unpaired, two-tail) was used to compare normally distributed groups of data. The Mann-Whitney test (unpaired data, two-tail) and Wilcoxon Signed Rank test (paired data) were used for non-normally distributed data. For comparison of cumulative distribution, Kolmogorov-Smirnoff test was used. For the comparison of multiple groups, one and two-way ANOVA test were used. The level of significance was set at p<0.05. Group measures are expressed as mean±SEM; error bars also indicate SEM.

### Results

Design and validation of viral vectors for the downregulation of GluK2 by RNA interference

In order to further confirm the specific role of GluK2/GluKKARs in the generation of epileptiform discharges (EDs) in brain slices, a virally-mediated RNA interference (RNAi) strategy was developed to downregulate the levels of GluK2/GluK5-containing KARs.

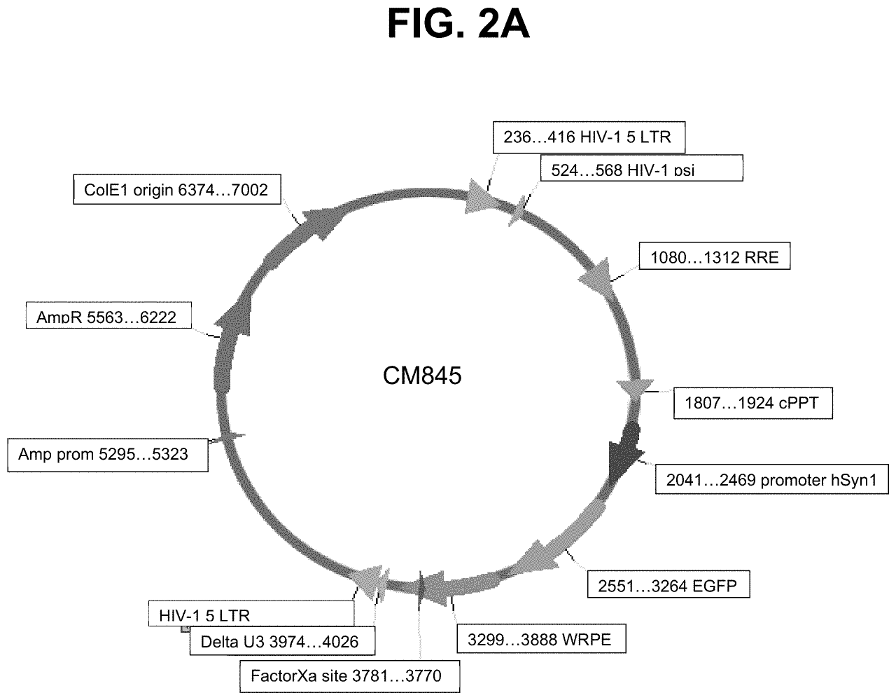

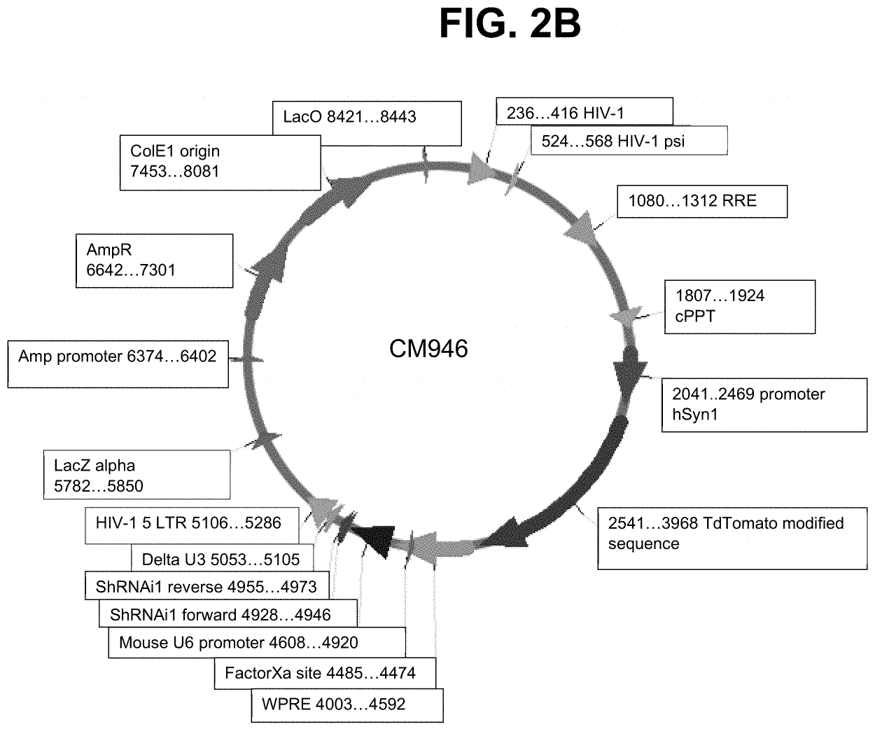

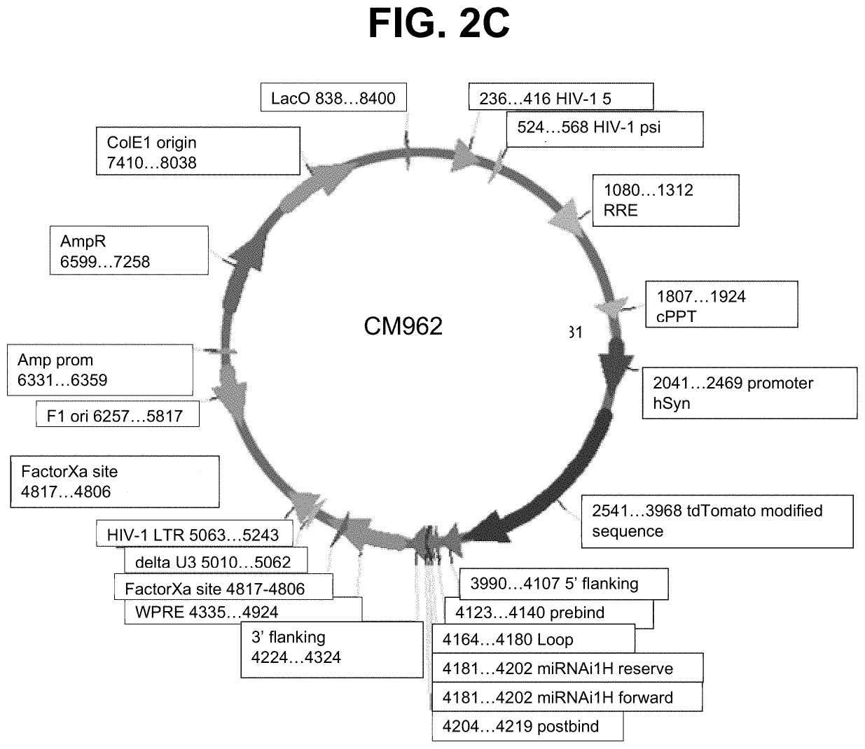

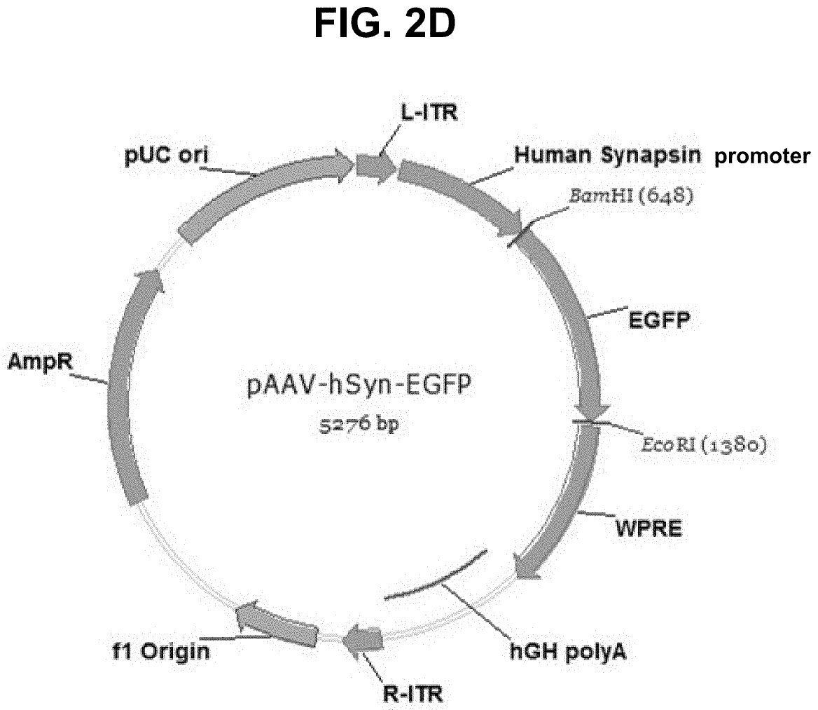

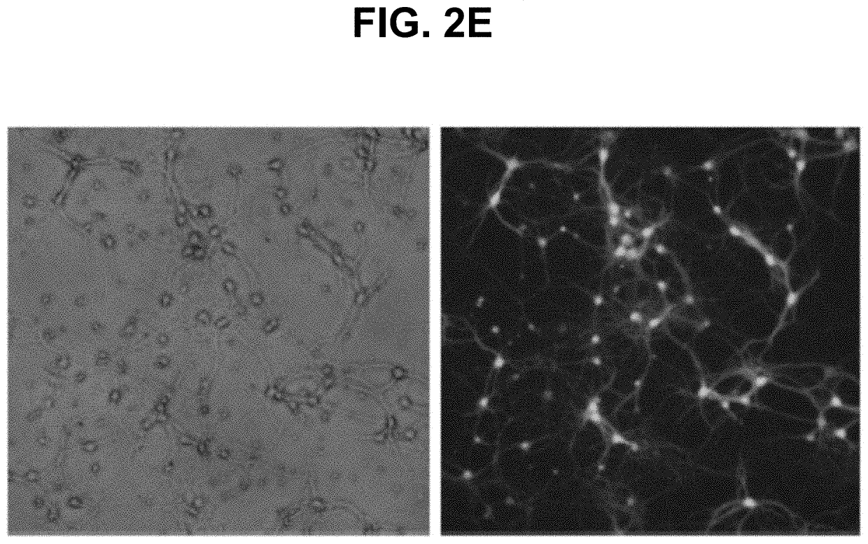

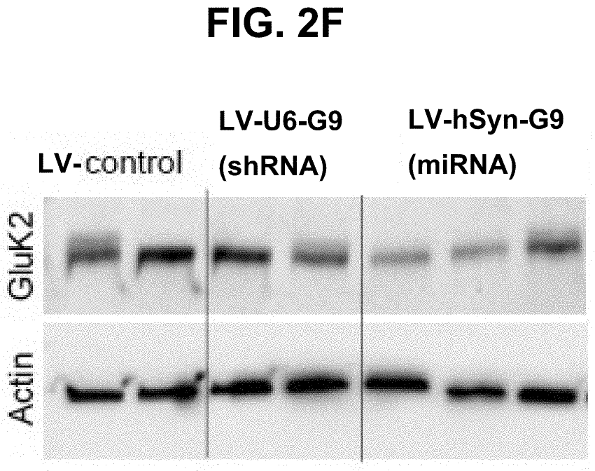

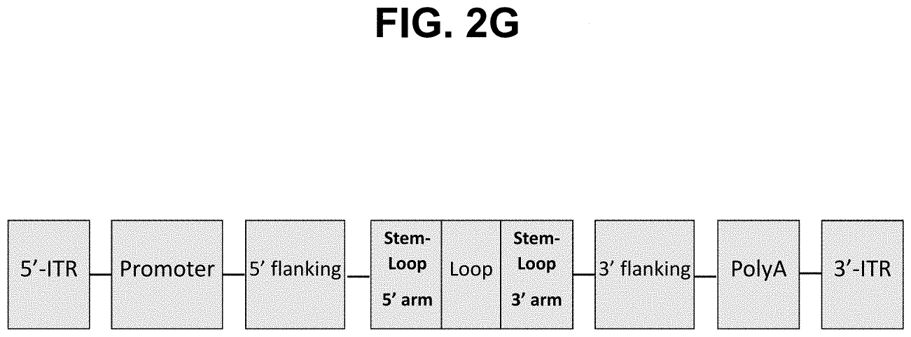

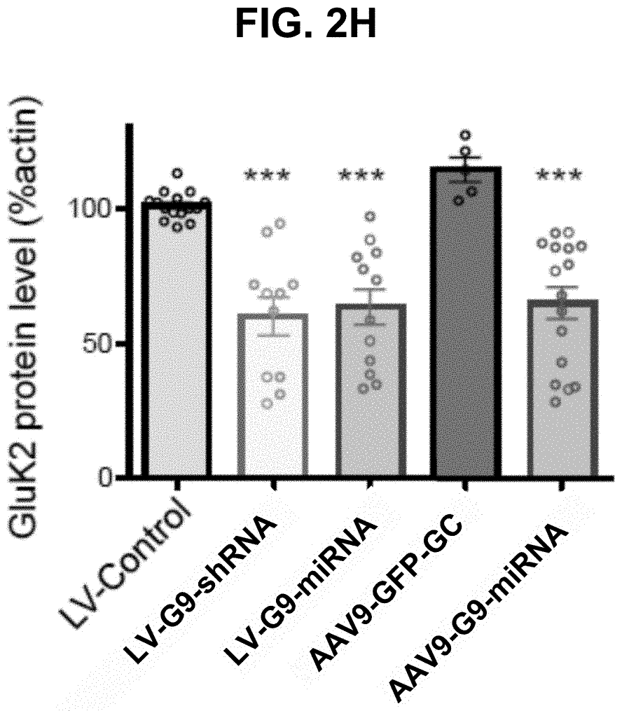

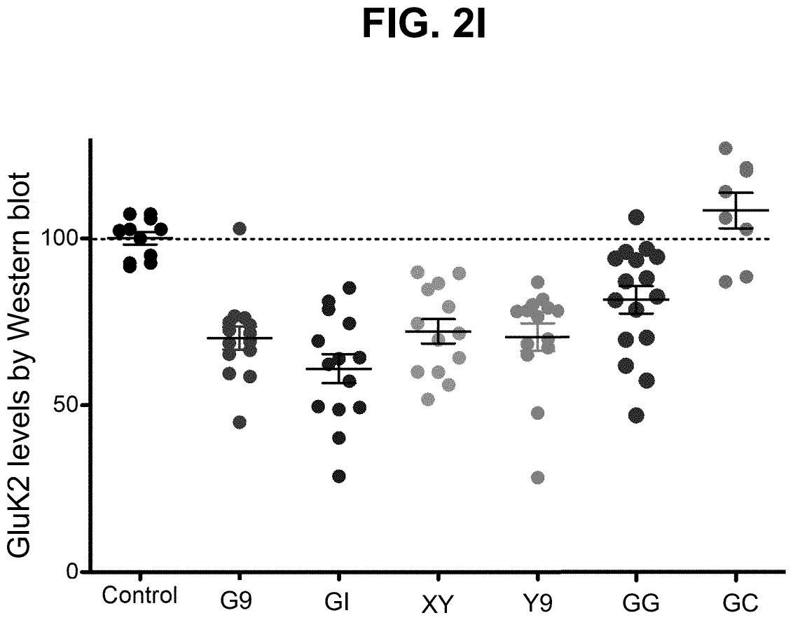

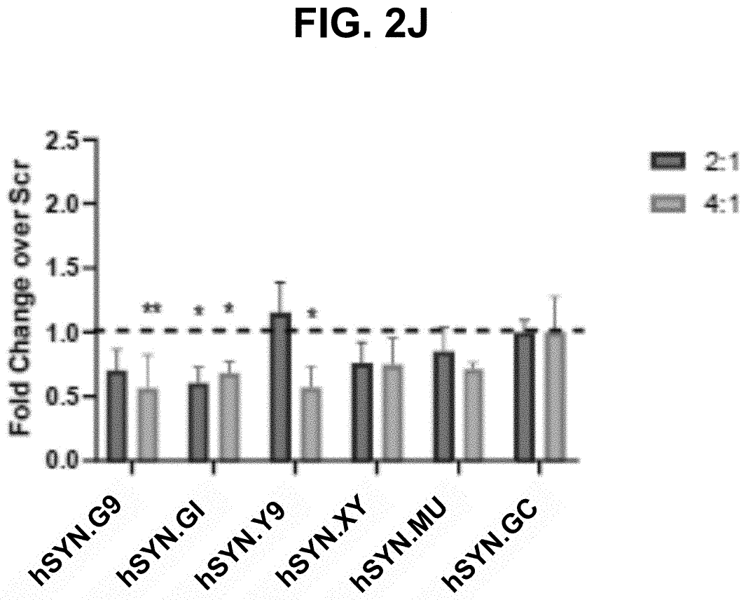

Several guide RNA sequences against the human Grik2 mRNA were designed using a Smart selection design (Birmingham et al. Nature Methods 9:2068-78, 2007). Initial experiments allowed the identification of one RNAi sequence (G9; SEQ ID NO: 68) potentially able to downregulate GluK2 expression in neurons. The efficacy of this RNAi sequence was assessed first using shRNA under the control of the U6 promoter and miRNA using a human miR-structure under the control of hSyn1 promoter. These constructs were delivered either by LV or AAV9 vectors (FIGS. 2A-2D) and were tested in rat hippocampal neurons and in rat hippocampal neurons by Western immunoblotting. Examples of lentiviral and AAV9 vectors used in these experiments are described in Table 12 below:

TABLE 12
Descriptions of lentiviral and AAV9 vector constructs

| | | | | | Viral |
|---|---|---|---|---|---|
| | | Plasmid | | | titer GC/ |
| ID | Construct | ID | Promoter | Tag | mL |
| LV- | LV.hSyn1.eGFP | CM845 | hSyn (SEQ | eGFP | 8E+08 |
| control/scramble | | | ID NO: 682) | | |
| (FIG. 2A) | | | | | |
| LV-U6-G9/LV- | LV.hSyn1.TdTomato.U6.shRNAi#h | CM946 | hSyn (SEQ | TdTomato | 7.9E+08 |
| G9-shRNA | | | ID NO: 683) | | |
| (shRNA; FIG. | | | for | | |
| 2B) | | | TdTomato; | | |
| | | | U6 (SEQ ID | | |
| | | | NO: 772) for | | |
| | | | shRNA | | |
| LV-hSyn-G9/LV- | LV.hSyn1.TdTomato.miRNAi#h | CM962 | hSyn (SEQ | TdTomato | 3.0E+09 |
| G9-miRNA | | | ID NO: 683) | | |
| (miRNA; FIG. | | | | | |
| 2C) | | | | | |
| AAV9-GFP-GC | AAV9.hSyn.EGFP_miR30.Scramble | N/A | hSyn (SEQ | eGFP | 9E+12 |
| | | | ID NO: 682) | | |
| AAV-G9-miRNA | AAV9.hSyn.miR30.miRNAi#h | N/A | hSyn (SEQ | N/A | 2.8E+13 |
| | | | ID NO: 683) | | |

Ten days after infection, the total amount of GluK2 protein was normalized to actin and determined in comparison with a scramble control construct (LV: TTTGTGAGGGTCTGGTC (SEQ ID NO: 771); AAV9: GC (SEQ ID NO: 101), as well as an AAV-CAG (SEQ ID NO: 737)-EGFP construct. The LV-hSyn (SEQ ID NO: 682)-EGFP construct allowed estimating the rate of infection of neurons with the LV vector, which was on the order of 40-50% in murine organotypic slices (FIG. 2E). An exemplary schematic of an AAV expression cassette used for AAV-mediated viral transduction in cells is shown in FIG. 2G). Antisense sequence G9 (SEQ ID NO: 68) was observed to be efficient in reducing the levels of total GluK2 protein, as an shRNA, as a miRNA and both in a LV vector and in an AAV9 vector (FIG. 2F). In rat hippocampal cultures (not shown), the different viral constructs encoding an anti-Grik2 mRNA sequence (G9; SEQ ID NO: 68) significantly reduced the level of GluK2 proteins, whereas the LV-scramble (SEQ ID NO: 771) showed no significant reduction. In rat hippocampal neurons (FIGS. 2H), different viral constructs encoding an anti-Grik2 sequence (G9; SEQ ID NO: 68) significantly reduced the level of GluK2 protein, whereas the LV-scramble (SEQ ID NO: 771) showed no significant reduction.

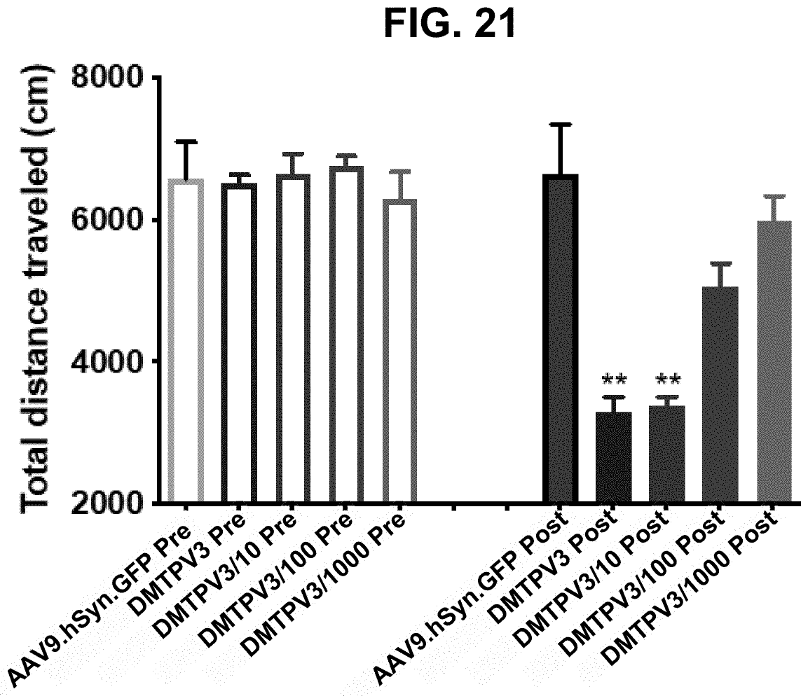

Several other viral vectors encoding anti-Grik2 sequences (G9 (SEQ ID NO: 68); GI (SEQ ID NO: 77); XY (SEQ ID NO: 83); Y9 (SEQ ID NO: 88); GG (SEQ ID NO: 91), and a negative control sequence (GC; UAAUGUUAGUCAUGUCCACcg; SEQ ID NO: 101) were tested for their ability to knockdown GluK2 protein expression (FIG. 21) in murine primary cortical neurons. Cultured cortical neurons were transduced with viral vectors encoding anti-Grik2 sequences at DIV3 at a multiplicity of infection 7.5×10⁴, with each construct tested in triplicates. Cells were lysed days after transduction. Table 13, below, provides a summary of the statistical analysis performed on the data presented in FIG. 21. All antisense constructs exhibited a statistically significant reduction in GluK2 protein levels, whereas the control sequence did not produce a significant knockdown.

TABLE 13
Comparative statistical analysis of the efficacy of various anti-Grik2
ASO sequences in knocking down GluK2 protein expression.

| | Mean | | Statistical | |
|---|---|---|---|---|
| | GluK2 | Range of | significance | |
| Treatment condition | level | GluK2 levels | level | p-value |
| G9 | 29.91 | 14.89 to 44.94 | **** | <0.0001 |
| (SEQ ID NO: 68) | | | | |
| GI | 39.07 | 24.05 to 54.10 | **** | <0.0001 |
| (SEQ ID NO: 77) | | | | |
| XY | 27.87 | 12.59 to 43.15 | **** | <0.0001 |
| (SEQ ID NO: 83) | | | | |
| Y9 | 29.61 | 14.58 to 44.63 | **** | <0.0001 |
| (SEQ ID NO: 88) | | | | |
| GG | 18.47 | 3.868 to 33.08 | ** | 0.0073 |
| (SEQ ID NO: 91) | | | | |
| GC | −8.327 | −25.65 to 9.000 | n.s. | 0.6425 |
| (SEQ ID NO: 101) | | | | |

In a separate set of experiments intended to evaluate the expression of Grik2 mRNA in endogenously expressing cells, induced pluripotent stem cell (iPSC)-derived glutamatergic neurons (iCelle® GlutaNeurons—FCDI) were transfected with plasmid encoding one of five Grik2 mRNA antisense oligonucleotides (G9 (SEQ ID NO: 68), GI (SEQ ID NO: 77), Y9 (SEQ ID NO: 88), XY (SEQ ID NO: 83), or MU (SEQ ID NO: 96)) or a scrambled control sequence (GC; SEQ ID NO: 101) under regulatory control of an hSyn promoter incorporated into a lipid-based transfection reagent. Transfection efficiency was compared using 2:1 and 4:1 rations of DNA : lipid-based transfection reagent. TaqManTm single-plex real-time quantitative polymerase chain reaction (RT-qPCR) was used to quantify levels of Grik2 knockdown in the GlutaNeurons in 384-well plates with three replicates post-transfection. GlutaNeurons were plated at a density of 17,500 cells/well (17.5k c/w). Grik2 mRNA expression was normalized to GAPDH signal. Transfection of plasmids encoding Grik2 antisense constructs was shown to significantly decrease Grik2 mRNA expression in GlutaNeurons after days for constructs containing G9 (SEQ ID NO: 68), GI (SEQ ID NO: 77), and Y9 (SEQ ID NO: 88) at a DNA-to-lipid reagent ratio of 4:1 (FIG. 2J). These findings demonstrate the effectiveness of Grik2 antisense oligonucleotides in knocking down expression of Grik2 mRNA in cells endogenously expressing the Grik2 gene.

GluK2 knockdown suppresses epileptiform activity in murine organotypic hippocampal slices

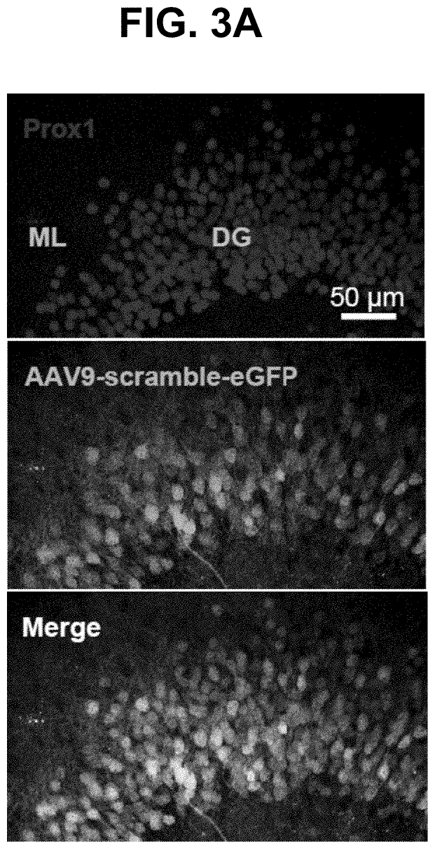

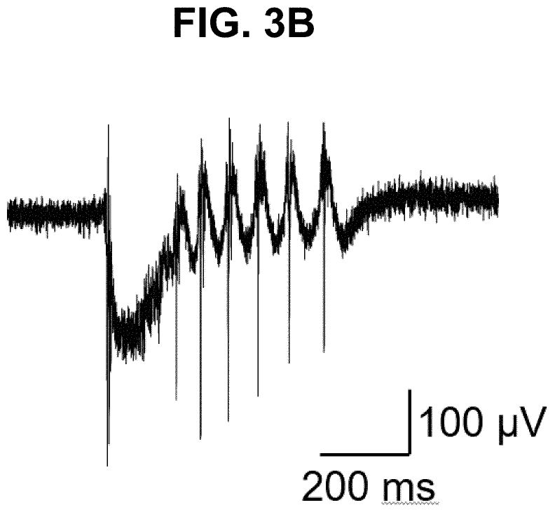

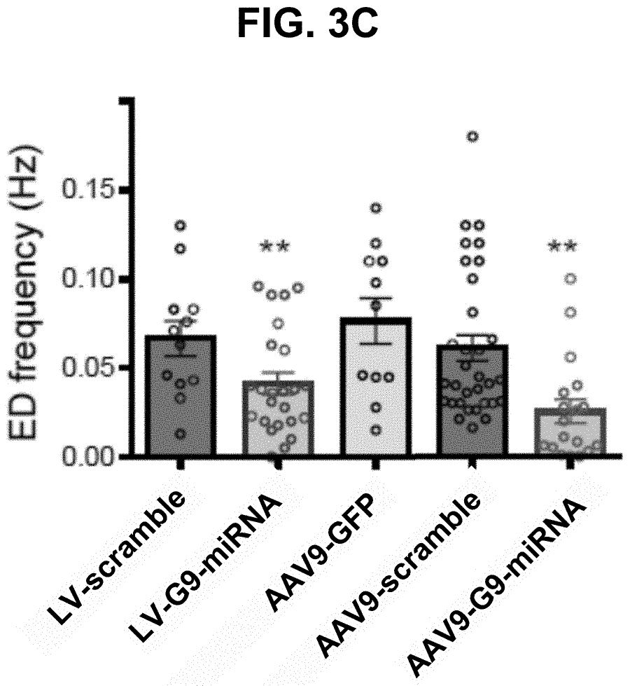

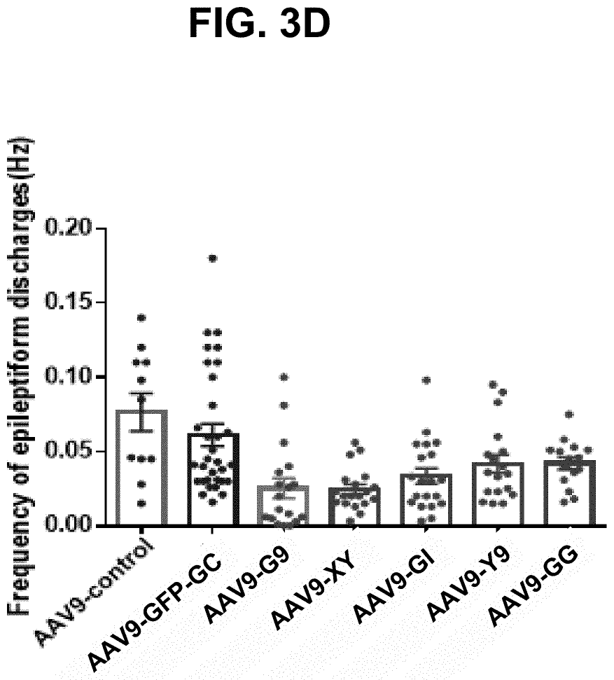

To confirm expression of the AAV9 constructs in target cells, Prospero Homeobox 1 (Prox1; Millipore) and green fluorescent protein (GFP) immunohistochemistry was performed on murine hippocampal slices transduced with AAV9-scramble-eGFP vectors. Indeed, extensive co-labeling of Prox1 and GFP was observed in DG neurons (FIG. 3A), indicating that AAV9 vectors can efficiently transduce murine DG neurons and express a polynucleotide of interest. The efficacy of LV or AAV constructs encoding a Grik2 antisense sequence (G9, SEQ ID NO: 68) as an shRNA or a miRNA in reducing the frequency of EDs recorded in murine hippocampal organotypic slices (exemplary extracelullar voltage trace shown in FIG. 3B) was tested in an extracellular medium generating hyperexcitability (see Methods) as previously described (Peret et al., 2014). LV-hSyn (SEQ ID NO: 682)—G9-miRNA (SEQ ID NO: 68) strongly reduced the frequency of EDs as compared with the control condition (LV-hSyn (SEQ ID NO: 682)-scramble; SEQ ID NO: 771) (0.041 ±0.006 Hz with LV-G9-miRNA, n=24; 0.066 ±0.009 Hz with LV-hSyn (SEQ ID NO: 682)-scramble (SEQ ID NO: 771), n=12, p=0.0246, Mann-Whitney test; FIG. 3C). Next, constructs with AAVs were tested since these viral vectors are commonly selected for human gene therapy. The AAV9 serotype was employed in these experiments. AAV9-hSyn (SEQ ID NO: 683)-G9 (SEQ ID NO: 68) efficiently reduced the frequency of EDs compared with control conditions (AAV9-hSyn (SEQ ID NO: 682)-GC; SEQ ID NO: 101) (0.025 ±0.007 Hz with AAV9-G9-miRNA, n=18; 0.061 ±0.007 Hz with AAV9-GFP-GC, n=32, p=0.0004, One Way ANOVA; FIG. 3C).

Additional AAV9 constructs encoding various anti-Grik2 ASO sequences were tested for their ability to suppress EDs in murine organotypic slices. Briefly, AAV9 vectors encoding various different anti-Grik2 ASO sequences were transduced in wild-type murine organotypic slices (see Methods) at DIVO at a viral titer of 9×10⁹genome copies/mL. Electrophysiological recordings in a hyperexcitable medium containing 5 μM gabazine were performed at DIV10-11. Inhibition of EDs generated in the disinhibited organotypic slice preparation was observed in five (out of five) constructs tested (i.e., G9 (SEQ ID NO: 76), XY (SEQ ID NO: 83), GI (SEQ ID NO: 477), Y9 (SEQ ID NO: 88), and GG (SEQ ID NO: 91; FIG. 3D). Summary of the statistical analysis of the data shown in FIG. 3D is provided in Table 14 below.

TABLE 14
Comparative statistical analysis of the efficacy of AAV9 vectors encoding anti-Grik2 RNA
sequences in suppressing epileptiform activity in murine organotypic brain slices.

| | | AAV9- | | | | | |
|---|---|---|---|---|---|---|---|
| | | GFP-GC | AAV9-G9 | AAV9- XY | AAV-GI | AAV9-Y9 | AAV9-GG |
| | AAV9- | (SEQ ID | (SEQ ID | (SEQ ID | (SEQ ID | (SEQ ID | (SEQ ID |
| | control | NO: 101) | NO: 68) | NO: 83) | NO: 77) | NO: 88) | NO. 91) |
| Mean | 0.07655 | 0.06116 | 0.02529 | 0.02459 | 0.03352 | 0.04183 | 0.04207 |
| SEM | 0.0127 | 0.007364 | 0.006768 | 0.00324 | 0.005286 | 0.005991 | 0.004087 |
| Sample size | 11 | 32 | 18 | 19 | 20 | 18 | 15 |
| Dunn′s multiple | Compared to AAV9-GFP-GC. | | | | | | |
| comparisons test | | | | | | | |
| p-value | >0.9999 | N/A | 0.0004 | 0.0008 | 0.0478 | 0.7569 | >0.9999 |

### Example3

### In Vivo Efficacy of Grik2-Targeting Antisense Constructs in a Murine Model of Temporal Lobe Epilepsy

Methods and Materials

To assess the in vivo efficacy of Grik2-targeting ASO agents, a pilocarpine-induced model of TLE was employed in mice in conjunction with treatment with a virally-encoded scrambled sequence (GC; SEQ ID NO: 101) under control of an hSyn promoter (SEQ ID NO: 682), or a virally encoded anti-Grik2 ASO agent (G9; SEQ ID NO: 68) under control of an hSyn promoter (SEQ ID NO: 683). On day 0 (DO), mice were given bilateral intrahippocampal injections of pilocarpine into dorsal (1 mL/hemisphere) and ventral (1 mL/hemisphere; 4 mL total/mouse) to induce status epilepticus. Mice were then given 3-4 weeks to allow the pathophysiological re-organization of the hippocampal circuit to occur. Seven days prior to injection with virally-encoded ASO agents (D60), mice were subjected to a behavioral assessment. As discussed herein, the neuroanatomical substrate for the etiology of TLE is the hippocampus, a brain region well-known for its critical role in memory and learning. To assess the effect of administering Grik2-targeting ASO agents into the hippocampus on learning and memory, pilocarpine-treated mice were first tested in a Novel Object Recognition (NOR) task for their ability to recognize novel and familiar objects. The basic structure of a NOR task involves presenting a mouse with two similar object during a first session and allowing the mouse to freely explore and familiarize itself with the two objects. As mice become familiar with the objects, the tendency to explore these previously novel objects diminishes. Prior to the start of a second session, one of the objects is replaced by a new object and the mouse is once again allowed to explore both objects. Generally, the latency to explore the new object provides a proxy for recognition memory, i.e., mice will exhibit a shorter latency to explore the novel object than the familiar object. 7 days after the initial behavioral assessment (D67), mice received bilateral intrahippocampal injections of 9×10¹² genome copies/mL of an AAV vector encoding either a scrambled control sequence (GC; SEQ ID NO: 101) with a green fluorescent protein (GFP) under control of an hSyn promoter (SEQ ID NO: 682) or a Grik2-targeting ASO sequence (G9; SEQ ID NO: 68) under control of an hSyn promoter (SEQ ID NO: 683). Details of the aforementioned ASO-encoding vectors are described in Table 15, below.

TABLE 15
Details of antisense constructs used in the study

| Construct | | Original titer | | Number of |
|---|---|---|---|---|
| ID | Genome content | (GC/mL) | Volume | GC/brain |
| G9 | hSyn.miR30.G9 | 9E+12 | 1 μL × 4 | 3.6E+10 |
| | (SEQ ID NO: 68) | | | |
| GC | hSyn.GFP.miR30.GC | 9E+12 | 1 μL × 4 | 3.6E+10 |
| | (SEQ ID NO: 101) | | | |

Seven days after receiving injections with the viral constructs (D82), mice were again subjected to the NOR task. 7 days after the post-injection NOR assessment (D89), mice were intrahippocampally implanted with electroencephalography (EEG) electrodes and electrographic seizures were recorded for five days from D96 to D103. Recordings were not continued any further due to COVID-19. Electrographic seizures were defined as paroxysmal events with an EEG amplitude of at least twice the EEG baseline and a duration of at least 6 seconds.

Results

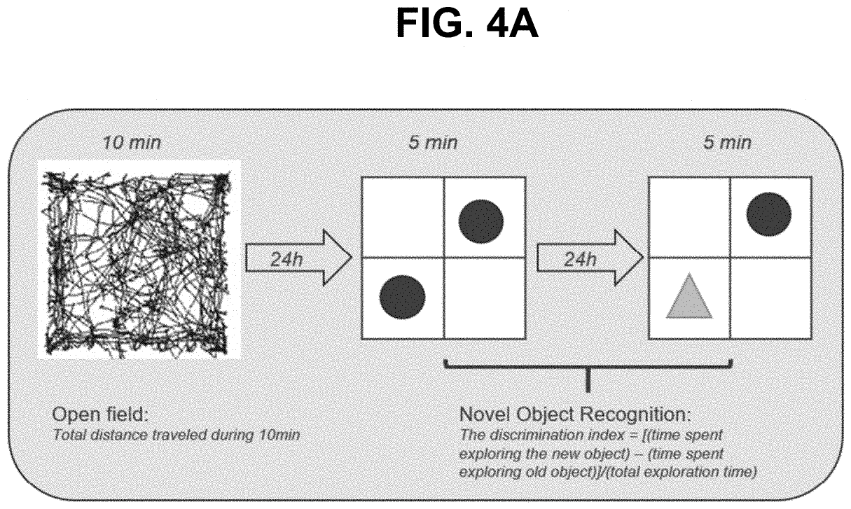

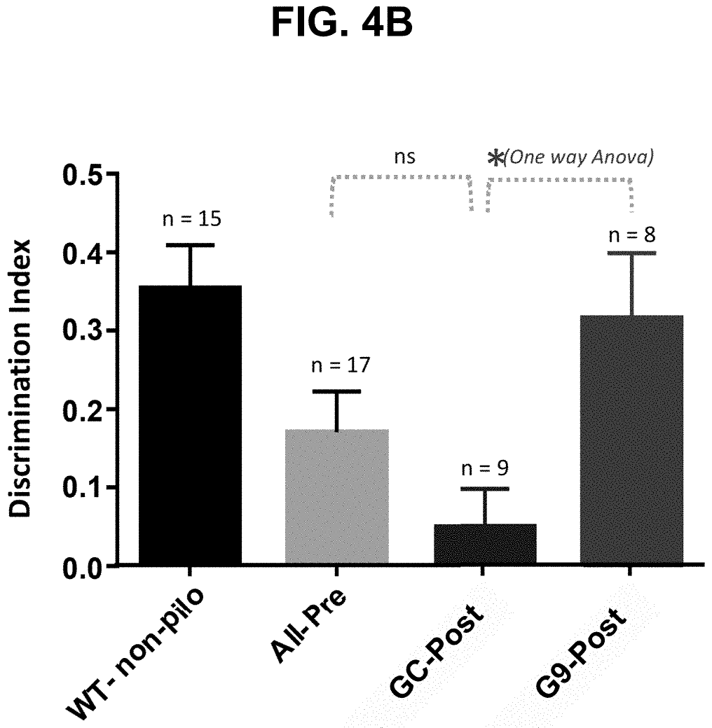

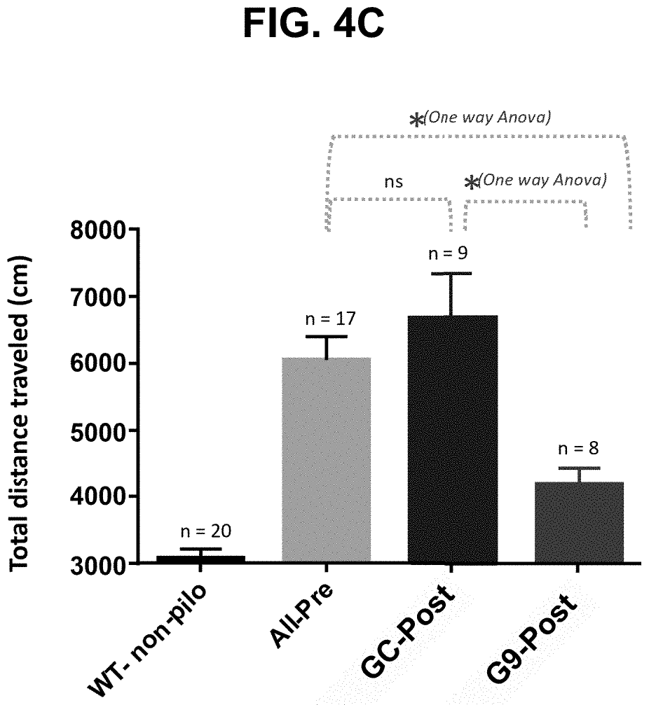

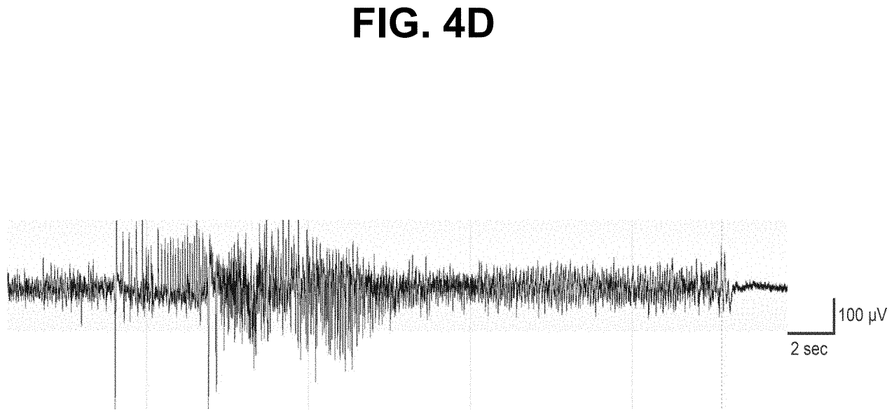

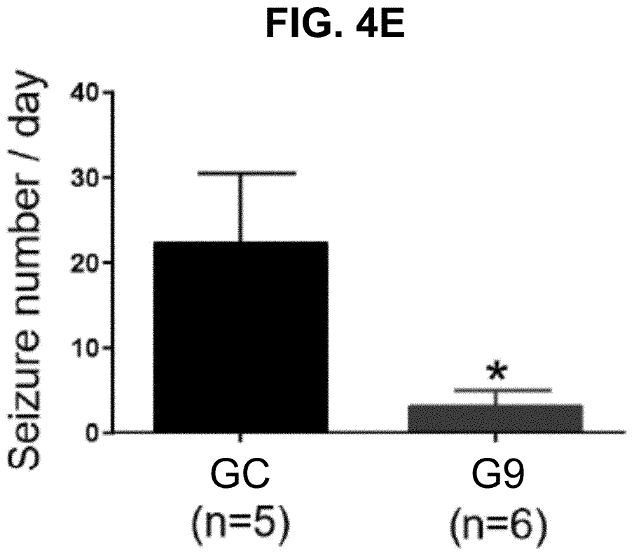

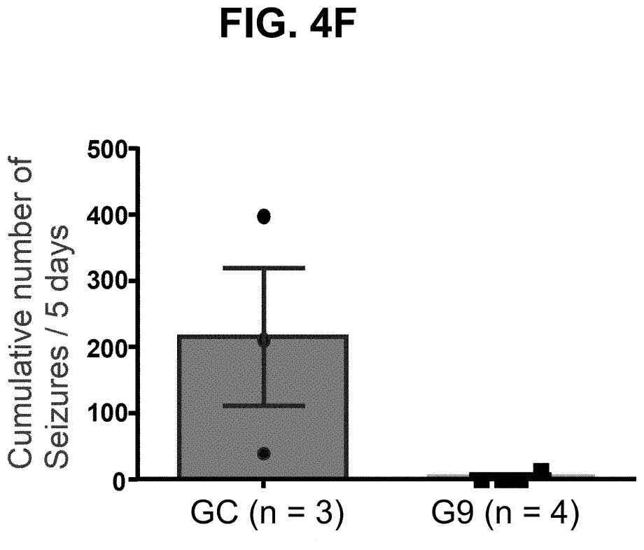

Patients diagnosed with TLE often exhibit other comorbidities in conjunction with epileptic seizures, such as, e.g., deficits in learning and memory, among others. To determine whether inhibition of Grik2 expression in vivo by administration of virally-encoded, Grik2-targeting antisense constructs, the NOR task (FIG. 4A) was performed on mice pre-treated with the pro-convulsant pilocarpine before and after injection with antisense constructs. As measured by the discrimination index (DI), which measures the difference between time spent exploring both objects during the test and training phase, and the total distance traveled, which is also a proxy for the tendency to explore the novel or the familiar object, control mice later to be treated with an AAV vector encoding a scrambled control sequence (GC; SEQ ID NO: 101) performed similarly to mice later to be treated with an AAV vector encoding an anti-Grik2 sequence (G9; SEQ ID NO: 76; FIG. 4B). Subsequent to viral vector injection, mice treated with the anti-Grik2 construct showed an improved discrimination of the novel object over the familiar object, as compared to control mice. Similarly, prior to vector injection, control mice and mice later to be treated with an anti-Grik2 sequence traveled a similar distance while exploring the familiar and novel objects. Subsequent to viral vector injection, mice treated with an anti-Grik2 construct (G9; SEQ ID NO: 68) showed reduced time traveled as compared to control mice (i.e., mice injected with viral vector encoding GC; SEQ ID NO: 101), which is indicative of a reduced tendency to engage in locomotor exploration and an increased tendency to explore the novel object (FIG. 4C). Quantitative analysis of the NOR task data is provided in Table 16 and Table 17, below.

TABLE 16
Quantitative summary of NOR task data

| | Non- | Pre- | GC-Post- | G9-Post- |
|---|---|---|---|---|
| | epileptic | injection | injection | injection |
| Number | 15 | 17 | 9 | 8 |
| of values | | | | |
| Mean | 0.3558 | 0.1705 | 0.04853 | 0.3162 |
| discrimination | | | | |
| index | | | | |
| SEM | 0.05569 | 0.05334 | 0.05029 | 0.08498 |

TABLE 17
Significance analysis of the data in Table 16 (p-values; ANOVA)

| | |
|---|---|
| G9-Post-injection vs. GC-Post-injection | p < 0.05 |
| G9-Post-injection vs. pre-injection | p < 0.05 |
| GC-Post-injection vs. pre-injection | p > 0.05 |

Spontaneous electrographic seizures were subsequently recorded from mice implanted with intrahippocampal EEG electrodes. An exemplary voltage trace of an electrographic seizure is provided in FIG. 4D. Mice treated with AAV vectors encoding Grik2-targeting ASO agents (G9; SEQ ID NO: 68) exhibited a significant reduction in cumulative seizure duration (FIG. 4E) and cumulative number of seizures (FIG. 4F) over days relative to control mice receiving a scrambled sequence (GC; SEQ ID NO: 101). A quantitative summary of the aforementioned experiment is provided in Table 18, below.

TABLE 18
Quantitative summary of EEG data

| | GC | G9 |
|---|---|---|
| Number of values | 5 | 6 |
| Mean number of | 22.17 | 2.988 |
| seizures per day | | |
| SEM | 8.365 | 2.038 |

Combined, these findings indicate that inhibition of expression of Grik2 mRNA using virally-encoded, Grik2-targeting is effective at reducing seizure frequency and duration in a murine model of TLE. Therefore, inhibitory Grik2-targerting ASO agents are a promising therapeutic avenue for the treatment of TLE in human patients.

### Example4

### Treatment of an Epilepsy in a Human Subject By Administration of a Viral Vector Encoding an Antisense Oligonucleotide Targeting a Grik2 Mrna

A subject, such as a human subject (e.g., a pediatric or adult subject) diagnosed as having an epilepsy (e.g., a TLE, such as, e.g., mTLE or ITLE), can be treated with a composition described herein to reduce one of more epilepsy symptoms including, but not limited to one or more of (e.g., 2 or more, 3 or more, 4 or more of): (a) risk of seizure recurrence; (b) reduction of excitotoxicity and associated neuronal cell death in the CNS; (c) restoration of a physiological excitation-inhibition balance in the affected region of the CNS; (d) reduction in one or more symptoms of a epilepsy (e.g., frequency, duration, or intensity of epileptic seizures, weakness, absence, sudden confusion, trouble understanding or producing speech, cognitive impairment, impaired mobility, dizziness, or loss of balance or coordination, paralysis, and emotional dysregulation), and (e) pathological sprouting of recurrent mossy fibers of dentate gyrus granule cells in the hippocampus. The method of treatment can optionally include diagnosing or identifying a patient as a candidate for treatment with a composition of the disclosure in a subject. The composition can include an ASO agent targeting a Grik2 mRNA or a nucleic acid vector (e.g., a viral vector, such as an AAV vector, e.g., an AAV vector having any one of the serotypes selected from AAV1, AAV2, AAV3, AAV4, AAVS, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAVIK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10 , AAV.HSC11, AAV.HSC12, AAV.HSC13, AAV.HSC14, AAV.HSC15, or AAV.HSC16 or a lentiviral vector) containing a polynucleotide encoding the same. Exemplary ASOs may have no less than 85% (e.g., at least 85%, 86%, 87%, 88%, 89%, 90%, 91%, 92%, 93%, 94%, 95%, 96%, 97%, 98%, 99%, or more) sequence identity to any one of the nucleic acid sequences of SEQ ID NOs: 1-100 or they may have the sequence of one or more of SEQ ID NOs: 1-100. Furthermore, the viral vector (e.g., AAV vector) may contain an expression cassette selected from the list provided in Table 9 or Table 10, which are incorporated by reference from U.S. Provisional Patent Application No.: 63/050,742 or any one of the constructs described in FIG. 30 or Table 9.

The subject can be administered the composition by any suitable means, including, e.g., intravenous, intraperitoneal, subcutaneous, or transdermal administration, or by way of administration directly to the central nervous system of the animal (e.g., stereotactic, intraparenchymal, intrathecal, or intracerebroventricular injection). The composition can be administered in a therapeutically effective amount, such as at a dose of 10⁵, 10⁶, 10⁷, 10⁸, 10⁹, 1010,1011, 1012, 1013, 1014_{, or 4015} genome copies (GC) per subject, at a dose of 10⁵, 10⁶, 10⁷, 10⁸, 10⁹, 1010,1011, 1012, 1013, or u 14 I GC/kg (total weight of the subject), at a dose of 10⁵, 10⁶, 10⁷, 10⁸, 10⁹, 1010, 1011, 1012, 1013, 1014, or 1015 LIL, per gram of patient's brain mass are administered. The subject's brain weight estimation is obtained from an MRI brain volume determination, which is converted to brain mass and utilized to calculate a precise dose of drug administered. Brain weights may also be estimated based on age range, using a published database. The agent can be administered bimonthly, once a month, once every two weeks, or at least once a week or more (e.g., 1, 2, 3, 4, 5, 6, or 7 times a week or more). The composition may be administered in combination with a second therapeutic modality, such as a second therapeutic agent (e.g., an anti-epileptic drug), surgical intervention (e.g., surgical resection, radiosurgery, gamma knife, or laser ablation), vagus nerve stimulation, deep brain stimulation, or transcranial magnetic stimulation.

The composition can be administered to the subject in an amount sufficient to decrease one or more of (e.g., 2 or more, 3 or more, 4 or more of): (a) risk of seizure recurrence; (b) reduction of excitotoxicity and associated neuronal cell death in the CNS; (c) restoration of a physiological excitation-inhibition balance in the affected region of the CNS; (d) reduction in one or more symptoms of a epilepsy (e.g., frequency, duration, or intensity of epileptic seizures, weakness, absence, sudden confusion, trouble understanding or producing speech, cognitive impairment, impaired mobility, dizziness, or loss of balance or coordination, paralysis, and emotional dysregulation), and (e) pathological sprouting of recurrent mossy fibers of dentate gyrus granule cells in the hippocampus by 10% or more (e.g., 10%, 20%, 30%, 40%, 50%, 60%, 70%, 80%, 90%, 95% or more). The above-listed symptoms of epilepsy may be assessed using standard methods, such as neurological examination, electroencephalogram, magnetoencephalogram, CT scan, PET scan, fMRI scan, videography, and visual observation. Measures of epilepsy symptoms from before and after administration of the composition can be compared to evaluate the efficacy of the treatment. A finding of a reduction in the symptoms of epilepsy described above indicates that the composition has successfully treated the epilepsy in the subject.

### Example5

### Design of Antisense Expression Constructs Targeting Grik2 Mrna Using Microrna Scaffolds

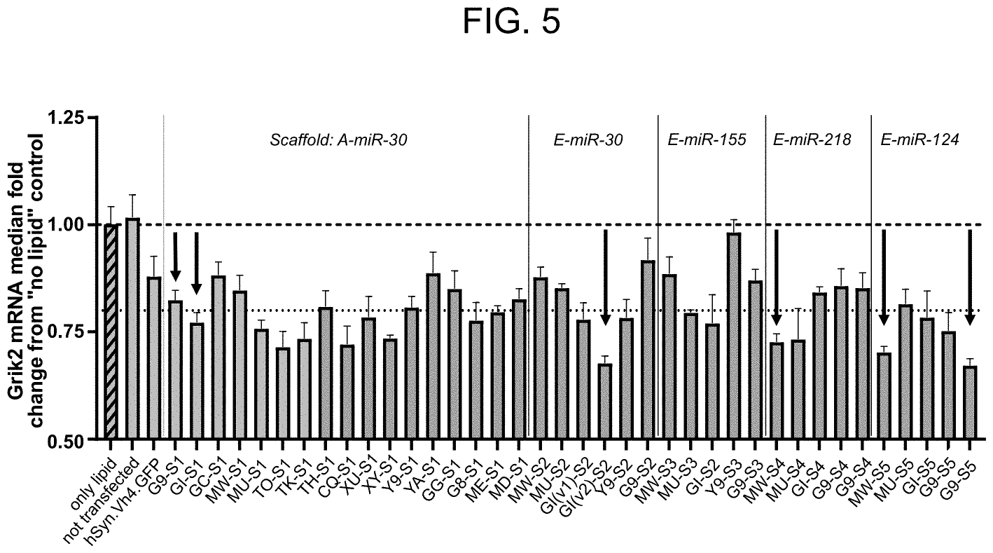

Additional constructs were designed, incorporating other described guides into an A-miR-scaffold (S1), based on the previously described selection criteria from the siRNA screen described in Example 1 A and Example 1B (FIGS. 6A-6E). A subset of these additional guide sequences were incorporated into additional miRNA “scaffolds” (containing a 5′ flanking region, loop sequence and 3′ flanking region from an endogenous miRNA) to improve mature miRNA expression and processing (S2=15 E-miR-scaffold; S3=E-miR-155 scaffold; S4=E-miR-218-1 scaffold; and S5=E-miR-124-3 scaffold). This larger panel of miRNA-expressing plasmid, under the control of the hSyn promoter (SEQ ID NO: 790), was transfected into induced pluripotent stem cell (iPSC)-derived glutamatergic neurons (GlutaNeurons) and evaluated for their ability to reduce Grik2 mRNA levels by qPCR. When compared to non-transfected cells (dashed line) and using a median absolute deviation (MAD) of two to identify functional constructs (dotted line), the majority of constructs were determined to be functional (i.e., they exhibit knockdown Grik2 mRNA below MAD)(Figure 5). The tested antisense sequences included G9 (SEQ ID NO: 68), GI (SEQ ID NO: 77), MW (SEQ ID NO: 80), GU (SEQ ID NO: 96), TO (SEQ ID NO: 14), TK (SEQ ID NO: 74), TH (SEQ ID NO: 22), CQ (SEQ ID NO: 35), XU (SEQ ID NO: 51), XY (SEQ ID NO: 83), Y9 (SEQ ID NO: 88), YA (SEQ ID NO: 63), GG (SEQ ID NO: 91), G8 (SEQ ID NO: 92), ME (SEQ ID NO: 69), and MD (SEQ ID NO: 70). Of these, GI (SEQ ID NO: 77)-S2 (SEQ ID NO: 798), MW (SEQ ID NO: 80)-S4 (SEQ ID NO: 799), MW-S(SEQ ID NO: 800), and G9 (SEQ ID NO: 68)-S(SEQ ID NO: 801) were found to knockdown Grik2 mRNA to the highest degree and were therefore identified as the most functional constructs (i.e., 20% or greater knockdown).

An AAV vector prepared using a plasmid encoding a single miRNA may result in an improperly packaged AAV for the following reasons. First, pri-miRNA sequences are short (<200 bases) and, depending on promoter length, design of a transgene cassette with a single promoter controlling expression of a single miRNA may result in an AAV genome that is significantly shorter than the maximum packaging capacity of AAV (˜4.8 kb). It is therefore possible that a single capsid may be loaded with more than one vector if the anticipated full genome length is <2.4 kb (half the packaging capacity of AAV). This can be mediated by polymerase read-through without proper endonuclease nicking that allows for the production of AAV genome dimers (or trimers) that can then be packaged into the AAV capsid if they are of appropriate length. This subsequently introduces significant heterogeneity into the population of AAV vector particles, which renders manufacturing and characterization of a drug product significantly more difficult. Second, shRNA- and miRNA-based transgenes inherently have significant secondary structure due to the inclusion of the miRNA hairpin. It has been shown that these internal secondary structures within an AAV genome can function as a “false” ITR during AAV genome replication and packaging, resulting in undesired truncation events and a heterogeneous population of AAV vector particles containing a mixture of full and partial vectors.

We tested several constructs for their ability to produce properly packaged AAV. These strategies implemented in the design of these constructs overcome the challenges of improper AAV genome packaging noted above.

AAV vectors encoding synthetic miRNAs in a number of different lengths and formats were produced (FIGS. 6A-6E), genomes extracted and evaluated by alkaline gel electrophoresis to evaluate genome length and integrity (FIG. 7). The genome content of a vector prep produced from a plasmid encoding a single promoter and a single miRNA cassette (expected length: 1.5 kb) was found to be comprised of a mixture of singly (1.5 kb), doubly (3.0 kb), and triply (4.5 kb) packaged genomes, indicating that the short length of the promoter-miRNA transgene cassette allowed for packaging of monomeric, dimeric, and trimeric genomes, with only 57% of vectors being the desired monomer (by densitometry, Table 19).

TABLE 19
Percentage of singly-packaged vector genomes
as a function of genome length

| | | Estimated |
|---|---|---|
| | | percentage |
| | | of singly- |
| | Single | packaged, |
| | genome | full-length |
| Construct | length (kb) | genomes |
| pCis-hSyn (SEQ ID NO: 790)-G9 | 1.5 | 57 |
| (SEQ ID NO: 68).miR30(S1) | | |
| pCis-C1ql2 (SEQ ID NO: 791)-hSyn- | 2.8 | 97 |
| G9.miR30(S1) | | |
| pCis-scAAV9-FLIP-hSyn-G9.mir30(S1) | 2.6 | 65 |
| pCis-scAAV9-hSyn-G9.mir30(S1) | 2.6 | 56 |
| pCis-hSyn-(G9)₃.miR30(S1) | 2.3 | 30 |
| pCis-hSyn-miRNA1- | 2.3 | 30 |
| miRNA2-miRNA3.miR30(S1) | | |

When a second promoter was added upstream of the first (resulting in a tandem promoter and extending the full-length genome length to 2.9kb, slightly more than half of the packaging capacity of AAV, almost all vector prep genomes are full length (97%). The inclusion of a miRNA hairpin did not introduce truncation events in either of these two genome formats, as packaged genome lengths were all multiples of the monomeric, full-length form.

One method of increasing effective genome length is by producing AAV with double-stranded, self-complementary genomes, which doubles the effective length of the vector. This is achieved by introducing a mutant ITR (mITR), in this case the 5′ ITR. Two scAAV vectors (each with a single promoter and a single miRNA) were evaluated for genome integrity: one with the promoter adjacent to the wild-type (wt)ITR and one with the promoter adjacent to the mITR. The majority of vectors for each orientation were found to be full length (2.6kb 65 and 56%, respectively); however, there was evidence of significant genome truncation events, resulting in smaller molecular weight genome fragments, as well as larger molecular weight genomes likely comprised of partially-dimeric genomes (one full length genome +one partial truncated fragment, for example). A less prominent species at -5.2 kb was also apparent, evidence that full-length, dimeric scAAV genomes are also present.

Another method of increasing genome length while also increasing miRNA expression is the introduction of additional miRNA motifs (multiple copies of a single miRNA, single copies of multiple miRNAs, or a combination of these); expression of multiple miRNAs, producing distinct mature, functional miRNAs, also introduces the potential to target distal regions of a given mRNA. Two constructs with concatenated miRNAs were tested: one with three copies of G9 (SEQ ID NO: 68)-S1 and one with one copy each of G9-S1, GI (SEQ ID NO: 77)-S1, and MU (SEQ ID NO: 96)-S1, each resulting in an expected full-length genome of 2.2kb (FIG. 7). As these genomes fall short of being greater than half the packaging capacity of AAV, there was evidence of double packaging, with only 30% of the genome population being the desired, monomeric full-length genome. Additionally, a number of partial genomes were detected whose lengths were suggestive of recombination events between homologous sequences in the individual miRNA cassette flanking sequences due to the inclusion of S1 scaffold for all three cassettes, introducing significant heterogeneity into the overall genome population. These findings indicate that introduction of additional sequences into single-miRNA expression constructs to bring the vector length closer to maximal vector capacity reduces the resulting genome heterogeneity and more reliably produces full-length vectors.

### Example6

### Dual-Antisense Expression Constructs Targeting Grik2 mRNA are Singly Packaged in AAV9 Vectors

Based on the above findings in Example 5, homogeneity of the AAV genome population in this setting is driven by length as well as use of the ssAAV format. In order to build a construct meeting these requirements while avoiding the pitfalls of concatemerizing similarly-sequenced miRNAs, a two promoter, two miRNA cassette strategy was adopted. In this format, one promoter, exemplified here as hSyn (SEQ ID NO: 790), drives expression of a single miRNA in one scaffold, terminating in one polyA sequence, is followed by a second promoter, exemplified here as CaMKII, drives the expression of a second miRNA in a different scaffold, terminating in a second polyA sequence.

This approach minimizes the sequence homology within the genome and results in a genome that is sufficiently long to avoid packaging dimeric or trimeric genomes (˜3.7 kb, well within the length requirements for full-length, singly-packaged AAV). Based on the results of the plasmid screen shown in FIG. 7, four such “dual constructs” were made using combinations of unique miRNA-scaffolds of the top “hits” (DMTPV1-4).

DMTPV1 contained, from 5′ to 3′, a 5′ ITR sequence (SEQ ID NO: 746), hSyn promoter (SEQ ID NO: 790), E-miR-124-3 5′ flanking sequence (SEQ ID NO: 768), a sense passenger strand sequence that is complementary to the antisense sequence of G9 (SEQ ID NO: 68), E-miR-124-3 loop sequence (SEQ ID NO: 770), antisense guide sequence of G9, E-miR-124-3 3′ flanking sequence (SEQ ID NO: 769), bovine growth hormone (BGH) polyA sequence (SEQ ID NO: 793), CaMKII promoter sequence (SEQ ID NO: 802), E-miR-30 5′ flanking sequence (SEQ ID NO: 759), a sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), rabbit beta-globin (RBG) polyA sequence (SEQ ID NO: 792), and a 3′ ITR sequence (SEQ ID NO: 748)(Figure 8A).

DMTPV2 contained, from 5′ to 3′, a 5′ ITR sequence (SEQ ID NO: 746), hSyn promoter (SEQ ID NO: 790), E-miR-124-3 5′ flanking sequence (SEQ ID NO: 768), a sense passenger strand sequence that is complementary to the antisense sequence of G9 (SEQ ID NO: 68), E-miR-124-3 loop sequence (SEQ ID NO: 770), antisense guide sequence of G9, E-miR-124-3 3′ flanking sequence (SEQ ID NO: 769), BGH polyA sequence (SEQ ID NO: 793), CaMKII promoter sequence (SEQ ID NO: 802), E-miR-218 5′ flanking sequence (SEQ ID NO: 765), a sense passenger strand sequence that is complementary to MW (SEQ ID NO: 80), E-miR-218 loop sequence (SEQ ID NO: 767), antisense guide sequence of MW (SEQ ID NO: 80), E-miR-218 3′ flanking sequence (SEQ ID NO: 766), RBG polyA sequence (SEQ ID NO: 792), and 3′ ITR sequence (SEQ ID NO: 748)(Figure 8B). DMTPV3 contained from 5′ to 3′, a 5′ ITR sequence (SEQ ID NO: 746), hSyn promoter (SEQ ID NO: 790), E-miR-30 5′ flanking sequence (SEQ ID NO: 759), a sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), BGH polyA sequence (SEQ ID NO: 793), CaMKII promoter sequence (SEQ ID NO: 802), E-miR-124-3 5′ flanking sequence (SEQ ID NO: 768), a sense passenger strand sequence that is complementary to the antisense sequence of G9 (SEQ ID NO: 68), E-miR-124-3 loop sequence (SEQ ID NO: 770), antisense guide sequence of G9, E-miR-124-3 3′ flanking sequence (SEQ ID NO: 769), RBG polyA sequence (SEQ ID NO: 792), and 3′ ITR sequence (SEQ ID NO: 748)(Figure 8C).

DMTPV4 contained, from 5′ to 3′, a 5′ ITR sequence (SEQ ID NO: 746), hSyn promoter (SEQ ID NO: 790), E-miR-30 5′ flanking sequence (SEQ ID NO: 759), a sense passenger strand sequence that is complementary to GI (SEQ ID NO: 77), E-miR-loop sequence (SEQ ID NO: 761), antisense guide sequence of GI (SEQ ID NO: 77), E-miR-30 3′ flanking sequence (SEQ ID NO: 760), BGH polyA sequence (SEQ ID NO: 793), CaMKII promoter sequence (SEQ ID NO: 802), E-miR-124-3 5′ flanking sequence (SEQ ID NO: 768), a sense passenger strand sequence that is complementary to the antisense sequence of MW (SEQ ID NO: 80), E-miR-124-3 loop sequence (SEQ ID NO: 770), antisense guide sequence of MW (SEQ ID NO: 80), E-miR-124-3 3′ flanking sequence (SEQ ID NO: 769), RBG polyA sequence (SEQ ID NO: 792), and 3′ ITR sequence (SEQ ID NO: 748)(Figure 8D).

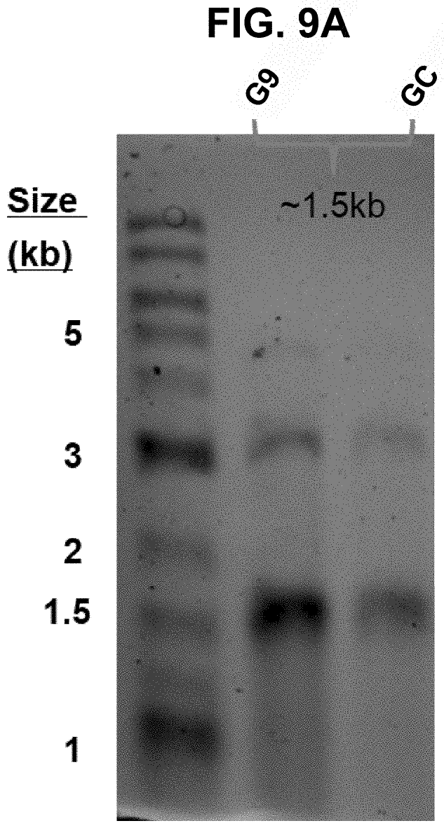

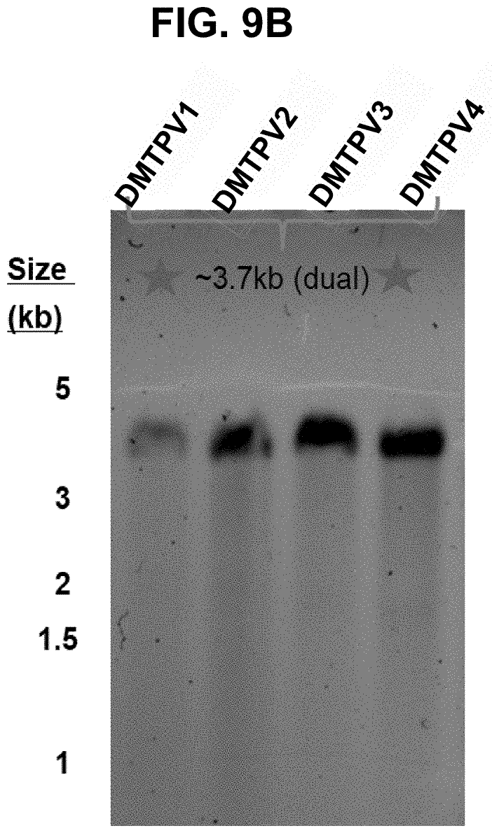

These dual constructs were produced as AAV9 vectors, and the impact of the two promoter, two miRNA cassette strategy on genome integrity was evaluated. When compared to two single promoter, single miRNA vectors (full length =1.5 kb, displaying evidence of dimeric and trimeric packaging), the genome content of these dual construct vectors is shown to be homogenous as evidenced by the presence of a single band at the expected full-length size of −3.7 kb (FIG. 9A Figure and 9B). Therefore, the introduction of a second promoter and a second miRNA cassette is an effective method of increasing the length of the AAV genome such that genomes are packaged as monomers while maintaining genomic integrity and avoiding truncation events and allowing for the expression of multiple miRNAs with the goal of improving targeting efficiency and total miRNA expression.

### Example7

### In Vitro Efficacy of Grik2 Mrna Knockdown Using a Combination of Two Distinct Anti-Grik2 Mirna Sequences

We first tested whether two different miRNA guides, when expressed in the same vector system or as two separate vectors, can mediate efficient, if not equivalent, knockdown of Grik2 expression. Four plasmid constructs were selected to be combined into dual construct vectors (based on findings in FIG. 7). The miRNA components of the dual construct vectors were transfected singly or in combination with each other in GlutaNeurons (keeping total quantity of plasmid transfected constant), and Grik2 mRNA levels were measured by qPCR (FIG. 10). When transfected together, combinations of anti-Grik2 5 miRNAs (both under the control of the synapsin promoter) were able to mediate robust Grik2 mRNA knockdown, supporting the use of vectors expressing more than one unique guide against Grik2.

We then tested the ability of anti-Grik2 miRNA-encoding vector preparations containing different, single miRNA cassettes with a single promoter (hSyn) for their ability to suppress epileptiform activity, as follows:

Mouse Hippocampal Organotypic Slices:

Organotypic slices were prepared from WT Swiss mice (P9-P10) using a Mcllwain tissue chopper as previously described (Peret et al. 2014). Slices were placed on mesh inserts (Millipore) inside culture dishes containing 1 mL of the following medium: MEM 50%, HS 25%, HBSS 25%, HEPES mM, glucose 6.5 mg/mL and insulin 0.1 mg/mL. Culture medium was changed every 2-3 days and slices were maintained in an incubator at 37° C./5% CO₂. Pilocarpine (0.5 μM) was added to the medium at DIV and removed at 7 DIV; slices were utilized for experiments from 9 DIV to 11 DIV.

Viral Infection of Organotypic Slices

For infection of mouse organotypic slices, 1μL of medium (phosphate-buffered saline) containing

AAV9 constructs was dropped directly on slices at DIVO.

Electrophysiological Recordings

Mice organotypic slices were individually transferred to a recording chamber maintained at 30-32° C. and continuously perfused (2-3 mL/min) with oxygenated (95% 02 and 5% CO₂) ACSF in the presence of 5 μM gabazine (Sigma-Aldrich); ACSF contained (in mM): NaCl, 3.5 KCI, 1.2 NaH2PO4, 26 NaHCO3, 1.3 MgCl2, 2.0 CaCl2, and glucose (pH -7.4)(Sigma-Aldrich). Local field potential (LFP) recordings were made with a monopolar Nichrome wire placed in the granule cell layer of the dentate gyrus. DAM-80 amplifier was used for recording (low filter, 0.1 Hz; highpass filter, 3 KHz; World Precision Instruments, Sarasota, FL); data were digitized (kHz) with a Digidata 1440A (Molecular Devices) to a computer, and acquired using Clampex 10.1 software (PClamp, Molecular Devices). Signals were analyzed off-line using Clampfit 9.2 (PClamp) and MiniAnalysis 6.0.1 (Synaptosoft, Decatur, GA).

Statistics

All values are given as means ±SEM. Statistical analyses were performed using GraphPad Prism (GraphPad software 5.01).

Test Constructs: G9, GI, G9-rGI

The tested vectors, G9 and GI (see Table 20, below), correspond to a miRNA sequence delivered by the AAV9 viral vector under the hSyn promotor. The control vector, GC, included GFP and scramble a miRNA sequence (GC, SEQ ID NO: 101) delivered by the AAV9 viral vector under control of the hSyn promotor.

TABLE 20
Vector details for slice electrophysiology experiments

| | | Original titer | Volume/ | Final titer |
|---|---|---|---|---|
| Virus # | Genome content | (GC/mL) | slice/mL | (GC/mL) |
| GC | hSyn.GFP.miR30.scr2 | 9E+12 | 1 μL | 9E+9 |
| G9 | hSyn.miR30.hu1010 | 9E+12 | 1 μL | 9E+9 |
| GI | hSyn.miR30.amiR38 | 6.50E+12 | 1 μL | 6.50E+9 |
| G9 + | hSyn.miR30.hu1010 + | 4.5E+12+ | 1 μL | 9E+9 |
| GI | hSyn.miR30.amiR38 | 4.5E+12 | | |

G9- and GI-encoding constructs, when mixed (equal parts, same total vector copies as those delivered singly) and applied to mouse organotypic slices were shown to reduce epileptiform activity to at least an equivalent degree as each vector prep individually (FIG. 11 and Tables 21 and 22, below).

TABLE 21
Slice electrophysiology data summary

| | GC | G9 | GI | G9 + GI |
|---|---|---|---|---|
| Number of slices | 32 | 39 | 27 | 16 |
| Mean # of | 0.06116 | 0.02377 | 0.02989 | 0.02008 |
| epileptiform | | | | |
| discharges | | | | |
| SEM | 0.007364 | 0.0037 | 0.004403 | 0.002089 |

TABLE 22
Statistical analysis of data in
Table 21 using ANOVA

| Group comparison | p-value |
|---|---|
| G9 vs. GC | p < 0.0001 |
| GI vs GC | p < 0.001 |
| G9 + GI vs GC | p < 0.0001 |

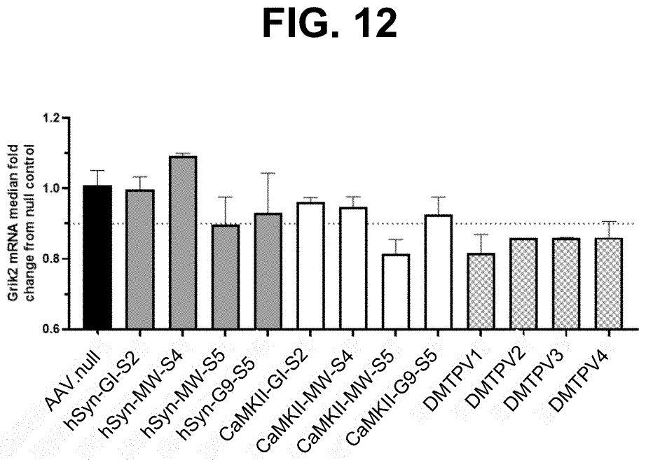

The ability of dual-miRNA AAV9 vectors to mediate Grik2 mRNA knockdown was then evaluated in the human iPSC-derived GlutaNeurons. Cells were seeded at a density of 17,500 cells/well in 384 well plates pre-coated with PEI and laminin, cultured for 11 days, transduced with AAV vectors at an MOI of 3×10⁵ GC/cell, and harvested for qPCR analysis eight days post-transduction. When compared to cells transduced with an AAV9.null vector (AAV9 containing a full-length genome that does not produce RNA, black bar) and using a median absolute deviation (MAD) of 2 to identify functional constructs (dotted line, value=0.9), it was determined that the dual AAV9 constructs (checked bars) mediate significant Grik2 mRNA knockdown, to a degree at least equivalent, if not to a higher degree, than their single construct component parts (hSyn components=gray bars, CaMKII components=white bars) at this timepoint with this MOI (FIG. 12).

### Example8

### In Vivo Efficacy of Grik2 mRNA-Targeting Single- and Dual-miRNA AAV9 Expression Constructs in a Pilocarpine-Induced Murine Model of Temporal Lobe Epilepsy

We used a murine pilocarpine-induced model of TLE to assess the efficacy of Grik2 mRNA-targeting dual-miRNA AAV expression vectors in vivo in conjunction with administration of various expression constructs following induction of status epilepticus.

### Material & Methods

### Ethics

All experiments were approved by the Institut National de la Sant6 et de la Recherche Medicale (INSERM) animal care and use committee and authorized by the Ministere de ('Education Nationale, de l′Enseignement Superieur et de la Recherche, following evaluation by a local ethical committee (agreement number APAFIS#9896-201605301121497v11) in accordance with the European community council directives (2010/63/UE).

### Chronic Epileptic Mice

Experiments were performed using male Swiss mice. The mice were kept at room temperature (20-22° C.) in a 12 h light/dark cycle with ad libitum access to food and water. Mice (30-g) were given scopolamine (1 mg/kg) subcutaneously (s.c.) minutes prior to intra-peritoneal administration of pilocarpine (300-350 mg/kg). A ramp protocol was used whereby animals were given an initial dose of 300 mg/kg followed by half-doses every minutes until seizures appeared. WT mice typically experienced at least two seizures prior to entering status epilepticus (SE). Caffeine (mg/kg) was administered minutes after the first seizure. Diazepam (mg/kg) was administered 1 hour after onset of status epilepticus (SE).

AAV injection

Epileptic mice (>2 months after SE) were anesthetized under isoflurane anesthesia (5% for induction and 2% for maintenance, under 100% 02). Body temperature was maintained using a heating pad, and they were placed in a stereotaxic frame. In the presence of local administration of lidocaine (2%), four holes were drilled to bilaterally inject AAV into the dorsal and ventral dentate gyrus of the hippocampus (AP -1.8 mm, ML ±1 mm, DV - 2 mm, and AP -3.3 mm, ML ±2.3 mm, DV - 2.5 mm). For each injection, a Hamilton syringe was used. After a 5-minute delay to allow the brain tissue to slide over the cannula, a volume =1.0 μL/injection site x 4 injection sites of a solution containing AAV was slowly infused (rate: 0.2 μL/minute). After infusion, the syringe was left in place for a further minutes in order to prevent backflow of the solution along the syringe track. During the recovery, the animals were given 5 mg/kg s.c. carprofen (Rimadyl®) 24 and 48 hours later.

### Locomotor Activity

The locomotion of non-epileptic and epileptic mice (>2 months after SE) was evaluated 1 week before and 2 weeks after AAV injection. Mice were transferred to the behavior analysis room 1 day prior experiments for habituation to the environment; the mice were kept at room temperature (20-22° C.) in a 9:00-18:00 light/dark cycle with ad libitum access to food and water. All materials that have been in contact with the animal tested were washed with acetic acid thereafter in order to prevent olfactory cues. First, spontaneous exploration behavior was tested with the open field test. Briefly, the mice were placed into the center of a 50×50×50 cm blue polyvinyl chloride box for min, and the trajectories were recorded with a video camera connected to a tracking software EthoVision Color (Noldus, The Netherlands); the speed and the total distance covered by the mice during 5-minute exploration were analyzed.

### EEG

Epileptic mice (>2 months after SE) were implanted with one depth wire electrode 3 weeks after AAV injection; as described above, surgeries were performed under isoflurane anesthesia. The electrodes were placed stereotaxically into the dentate gyrus (DG) (Paxinos and Watson coordinates from bregma: AP −2.55 mm, ML +1.65 mm, DV −2.25 mm). An additional screw, placed over the cerebellum, served as ground electrode. The electrode and the screw were secured on the skull with dental cement. During the recovery, the animals were given mg/kg s.c. carprofen (Rimadyl®) 24 and 48 hours later. EEG (amplified (1000×), filtered at 0.16-97 Hz pass, acquired at 500 Hz) was monitored using a telemetric system (Data Sciences International, St. Paul, Minn.) for days, 24 hours per day. Intrahippocampal EEG traces represented the difference in potential between the electrode inserted into the DG and an electrode positioned above the cerebellum.

### Statistics

All values are given as mean±SEM. Statistical analyses were performed using Graphpad Prism 7 (GraphPad Software, La Jolla, Calif.). For between-group comparisons, raw data were analyzed by a Mann-Whitney test. For multiple group comparisons, raw data were analyzed by a one-way ANOVA test. The level of significance was set at p<0.05. *p<0.05, **p<0.01, ***p<0.001.

### Results

### Test Items

The tested vector, G9 (SEQ ID NO: 68), GI (SEQ ID NO: 77) corresponds to a miRNA sequence delivered by the AAV9 viral vector under the hSyn promotor (SEQ ID NO: 790). The GC (SEQ ID NO: 101) and AAV9.GFP correspond to control vectors; GC and AAV9.GFP include GFP and scramble miRNA sequences, and a GFP sequence, respectively, which are delivered by the AAV9 viral vector under the hSyn promoter (see Table 23, below).

TABLE 23
Details for miRNA single constructs

| | | Original | | Number |
|---|---|---|---|---|
| | | titer | | of |
| Vector ID | Genome content | (GC/mL) | Volume | GC/brain |
| G9 | hSyn (SEQ ID NO: | 9E+12 | 1 μL × 4 | 3.6E+10 |
| | 790).miR30.G9 (SEQ ID | | | |
| | NO: 68) | | | |
| GI | hSyn.miR30.GI (SEQ ID | 6.5E+12 | 1 μL × 4 | 2.6E+10 |
| | NO: 77) | | | |
| GC | hSyn.GFP.miR30.GC | 9E+12 | 1 μL × 4 | 3.6E+10 |
| | (SEQ ID NO: 101) | | | |
| AAV9.GFP | hSyn.GFP | 5.11E+13 | 1 μL × 4 | 3.6E+10 |

Evaluation of the Efficacy of G9 and GI miRNA Constructs

The efficacy of G9 and GI were evaluated on hyperlocomotion, a behavioral marker of epilepsy, and the spontaneous recurrent seizures in chronic epileptic mice (>2 months following status epilepticus). In this set of experiments, GC was used as control vector.

Hyperlocomotion

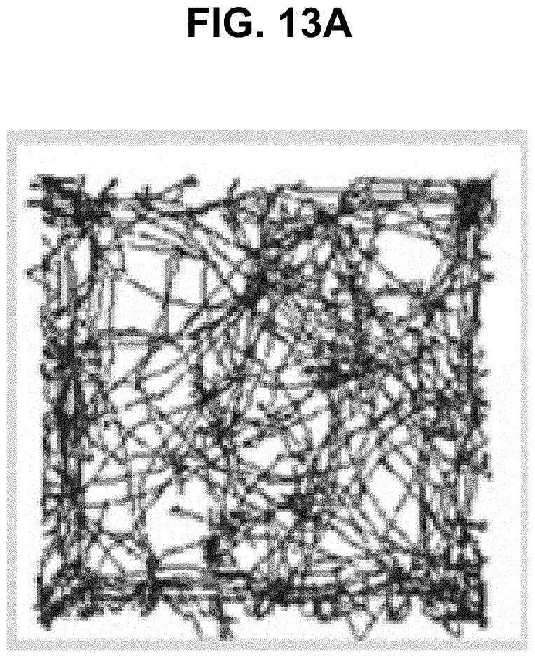

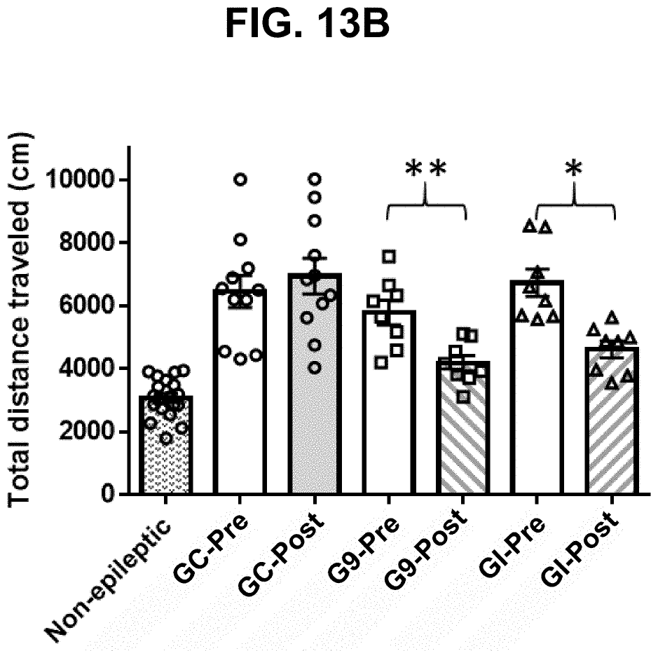

G9- and GI-encoding vectors demonstrated a significant decrease in hyperlocomotion toward levels similar to non-epileptic mice (see FIG. 13A and FIG. 13B). No significant change is observed with GC, the control vector (see Table 24, below).

TABLE 24
Total distance traveled in open field test

| | Non- | | | | | | |
|---|---|---|---|---|---|---|---|
| | Epileptic | Pre-GC | Post-GC | Pre-G9 | Post-G9 | Pre-GI | Post-GI |
| Number of mice | 20 | 11 | 11 | 8 | 8 | 8 | 8 |
| Mean distance | 3085 | 6450 | 6945 | 5789 | 4183 | 6730 | 4618 |
| traveled (cm) | | | | | | | |
| Std. Error of Mean | 135.4 | 595.4 | 658.6 | 393.6 | 243.3 | 430.6 | 265.4 |

EEG

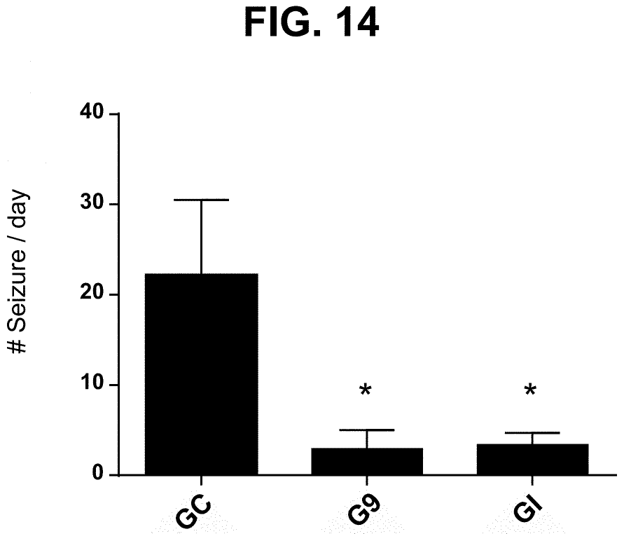

G9- and GI-encoding constructs demonstrated a suppression of epileptic seizures compared with GC-encoding construct, the control vector (see FIG. 14 and Table 25, below).

TABLE 25
Seizure numbers per day

| | GC | G9 | GI |
|---|---|---|---|
| Number of mice | 5 | 6 | 5 |
| Mean | 22.17 | 2.988 | 3.426 |
| Std. Error of Mean | 8.365 | 2.038 | 1.256 |

### Dose-Response of a G9-Encoding AAV9 Construct

A dose-response testing using G9 encoded in an AAV9 expression vector was performed in chronic epileptic mice (>2 months following SE). The efficacy of the dose response of G9 under different dilutions (see Table 26, below) was evaluated on hyperlocomotion, a behavioral marker of epilepsy, and on the spontaneous recurrent seizures. In this set of experiments, AAV9-GC was used as control vector.

TABLE 26
Details for miRNA constructs used in the study

| | | Original | | Number |
|---|---|---|---|---|
| | | titer | | of |
| Vector # | Genome content | (GC/mL) | Volume | GC/brain |
| G9 (SEQ ID | hSyn (SEQ ID NO: | 9E+12 | 1 μL × 4 | 3.6E+10 |
| NO: 68) | 790).miR30.G9 | | | |
| G9/10 | hSyn.miR30.G9 | 9E+11 | 1 μL × 4 | 3.6E+9 |
| G9/100 | hSyn.miR30.G9 | 9E+10 | 1 μL × 4 | 3.6E+8 |
| G9/1000 | hSyn.miR30.G9 | 9E+9 | 1 μL × 4 | 3.6E+7 |
| GC | hSyn.GFP. | 9E+12 | 1 μL × 4 | 3.6E+10 |
| | mir30.GC | | | |

Hyperlocomotion

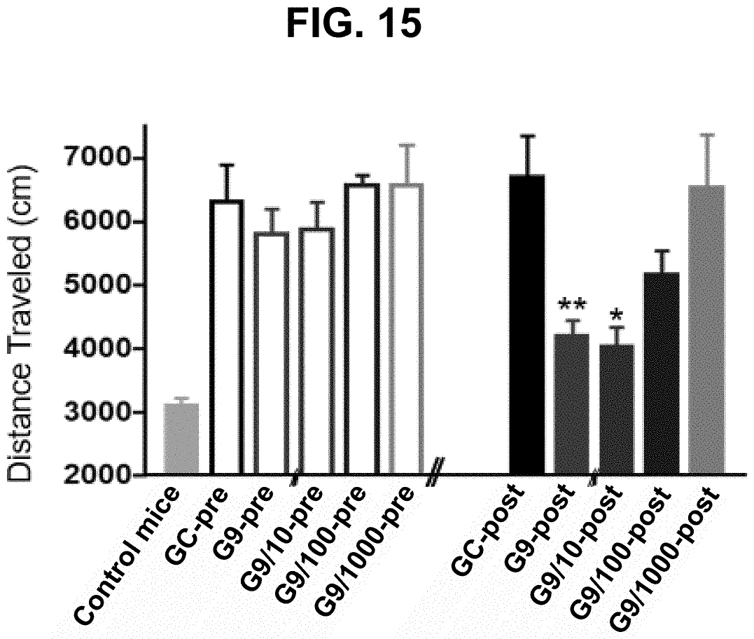

G9 demonstrated a dose response efficacy; we observed a similar efficacy of G9 and G9/10 and a loss of efficacy of G9/1000 when compared before and after injection and with GC (see FIG. 15 and Table 27, below).

TABLE 27
Total distance traveled

| | Pre- | Post- | Pre- | Post- | Pre- | Post- | Pre- | Post- | Pre- | Post- |
|---|---|---|---|---|---|---|---|---|---|---|
| | GC | GC | G9 | G9 | G9/10 | G9/10 | G9/100 | G9/100 | G9/1000 | G9/1000 |
| Number of mice | 9 | 9 | 8 | 8 | 5 | 5 | 5 | 5 | 5 | 5 |
| Mean distance (cm) | 6294 | 6676 | 5789 | 4183 | 5862 | 4015 | 6553 | 5154 | 6551 | 6522 |
| SEM | 595.4 | 658.6 | 393.6 | 243.3 | 441.9 | 312.9 | 162.9 | 378.5 | 636.3 | 831.7 |

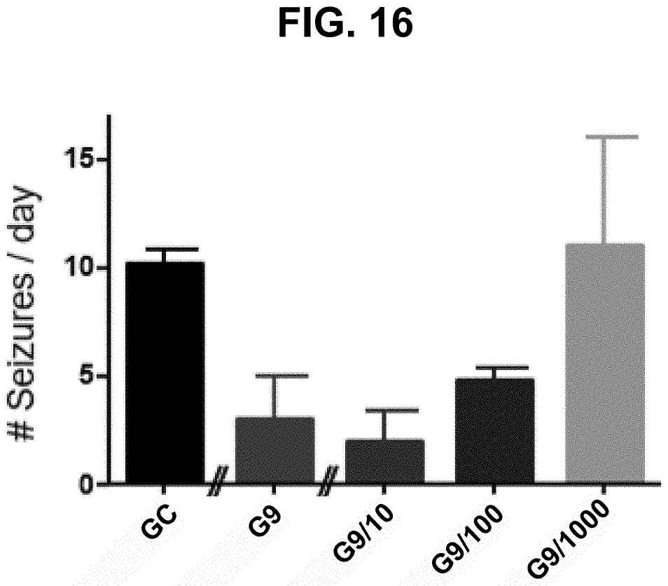

The initial data suggests a dose-dependent suppression of epileptic seizures by G9-encoding constructs in mice (see FIG. 16 and Table 28, below).

TABLE 28
Seizure numbers per day

| | G9 | G9/10 | G9/100 | G9/1000 |
|---|---|---|---|---|
| Number | 6 | 4 | 2 | 2 |
| of mice | | | | |
| Mean | 2.988 | 2.01 | 4.78 | 11 |
| SEM | 2.038 | 1.356 | 0.62 | 5 |

Dual-miRNA AAV9 Expression Constructs

In order to determine the efficacy of Grik2 mRNA-targeting dual-miRNA AAV expression vectors in vivo, a murine pilocarpine-induced model of TLE was employed in conjunction with administration of various single-siRNA and dual-miRNA AAV expression constructs following induction of status epilepticus.

The general structure of the experiment was as follows. First, status epilepticus was induced in mice via systemic administration of pilocarpine and subsequently terminated with diazepam. Mice were given about one week to recover. Over the next three weeks, pilocarpine-treated mice were monitored for seizure development until they displayed a stable, spontaneous recurrence of epileptic seizures (about >2 months from time of pilocarpine treatment). Once this benchmark was reached, mice were tested for locomotion in an open field during the first week following the establishment of recurrent epileptic seizures. During weeks 2-3, mice were treated with one of multiple single-siRNA or dual-miRNA AAV expression vector. The single-siRNA expression vectors included a scrambled control siRNA sequence (GC, SEQ ID NO: 101), one of two Grik2-targeting sequences (G9, SEQ ID NO: 68; or GI, SEQ ID NO: 77), or a GFP transgene under control of an hSyn promoter (SEQ ID NO: 790). The dual-miRNA expression constructs included one of DSPTV1-4 constructs, described above and shown in detail in Table 29, below.

TABLE 29
Details for miRNA dual constructs

| | | Original titer | | Number of |
|---|---|---|---|---|
| Vector# | Genome content | (GC/mL) | Volume | GC/brain |
| DMTPV1 | hSyn.G9.E-miR-124- | 1.21E+14 | 1 μL × 4 | 3.6E+10 |
| (SEQ ID NO: 785) | 3.CaMKII.GI.E-miR-30 | | | |
| DMTPV2 | hSyn.G9.E-miR-124- | 1.29E+14 | 1 μL × 4 | 3.6E+10 |
| (SEQ ID NO: 786) | 3.CaMKII.MW.E-miR-218-1 | | | |
| DMTPV3 | hSyn.GI.E-miR-30.CaMKII.G9.E- | 1.45E+14 | 1 μL × 4 | 3.6E+10 |
| (SEQ ID NO: 787) | miR-124-3 | | | |
| DMTPV4 | hSyn.GI.E-miR-30.CaMKII.MW.E- | 1.62E+14 | 1 μL × 4 | 3.6E+10 |
| (SEQ ID NO: 788) | miR-124-3 | | | |

After about days post-treatment, locomotion was again assessed in mice treated with pilocarpine and one of several siRNA expression constructs using the open field test. DSPTV3 and DSPTV4 constructs exhibited significant suppression of pilocarpine-induced hyperlocomotion in mice,

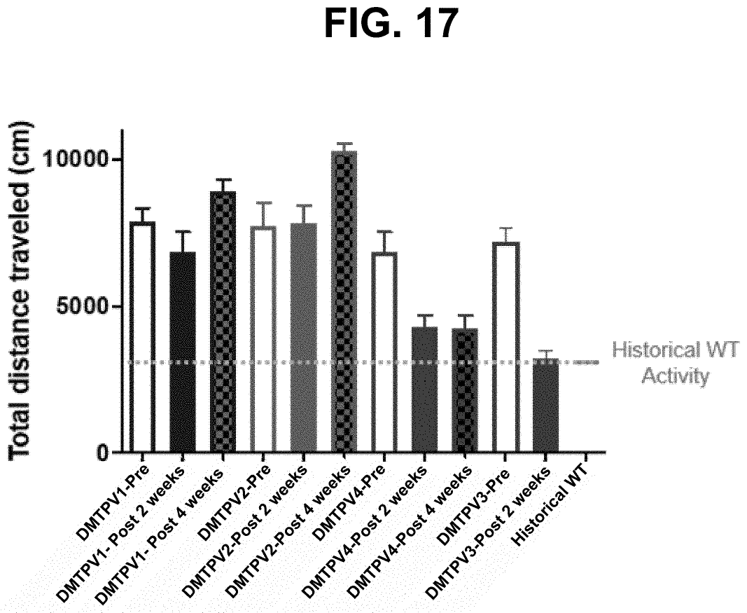

(FIG. 17 and Table 30, below). AAV9-GFP was used as control vector.

TABLE 30
Total distance traveled

| | | AAV9- | AAV9- | | | | | | | | |
|---|---|---|---|---|---|---|---|---|---|---|---|
| | Non- | GFP- | GFP - | DMTPV1- | DMTPV1- | DMTPV2- | DMTPV2- | DMTPV3- | DMTPV3- | DMTPV4- | DMTPV4- |
| | epileptic | pre | Post | Pre | Post | Pre | Post | Pre | Post | Pre | Post |
| Number of mice | 20 | 6 | 6 | 5 | 5 | 6 | 6 | 6 | 6 | 6 | 6 |
| Mean distance | 3085 | 6956 | 6630 | 7877 | 6860 | 7755 | 7857 | 7208 | 3249 | 6866 | 4304 |
| traveled (cm) | | | | | | | | | | | |
| Std. Error of Mean | 135.4 | 727.6 | 506.3 | 461.5 | 653.4 | 751.2 | 576.2 | 420.6 | 228.9 | 650.3 | 403.5 |

Correlation between Seizures and Hyperlocomotion

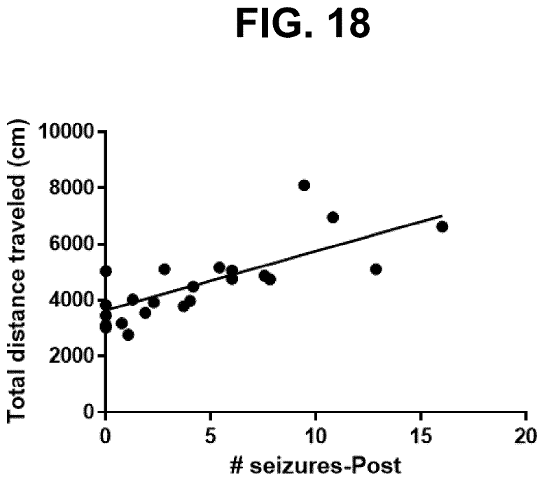

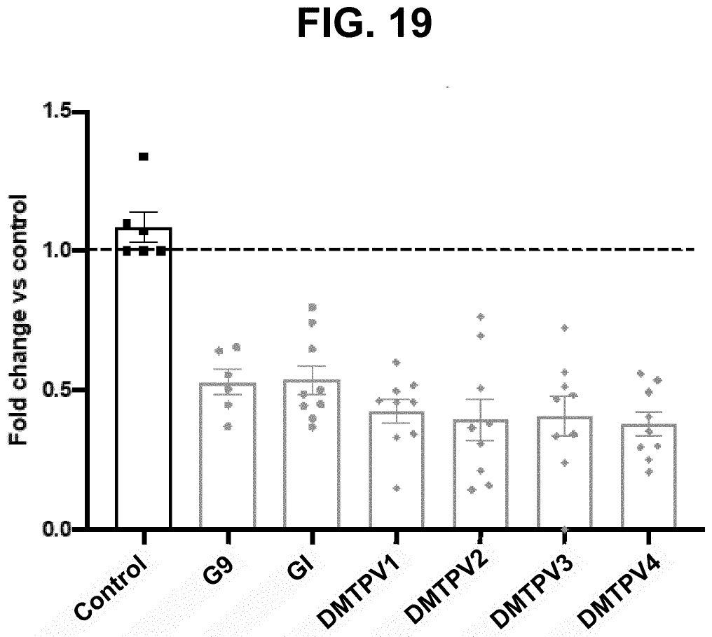

In order to draw conclusions about the anti-epileptogenic effects of Grik2-targeting, AAV-encoded miRNA constructs through measurement of hyperlocomotion in an open field test in mice, the correlation between the number of seizures and hyperlocomotion post-treatment was analyzed. Extreme values of seizures were excluded based on the outliers analysis ROUT (Q =1.000%). Under the aforementioned experimental conditions, there was a significant correlation (R² =0.7388, p<0.0001) between the number of epileptic seizures per day and the total distance traveled in an open field test by experimental mice (FIG. 18). Thus, measures of hyperlocomotion can be used as an index for epileptic status in mice, enabling a quick and humane approach to test the anti-epileptogenic effects of Grik2-targeting antisense constructs.

### Conclusion

These data demonstrate that Grik2 gene silencing, using AAV vectors carrying RNAi sequences targeting Grik2 mRNA, is an efficient strategy to prevent spontaneous chronic seizures in TLE.

### Example9

### In Vitro and In Vivo Efficacy of Vectors Encoding Dual Anti-Grik2 Constructs in Mice

In a first set of experiments intended to evaluate the efficacy of dual-miRNA encoding vectors containing anti-Grik2 sequences in cells endogenously expressing Grik2, GlutaNeurons were transduced with an AAV9 vector encoding a single anti-Grik2 miRNA sequence G9 (SEQ ID NO: 68) or GI (SEQ ID NO: 77), dual anti-Grik2 sequence-encoding vectors DMTPV1 (SEQ ID NO: 785), DMTPV2 (SEQ ID NO: 786), DMTPV3 (SEQ ID NO: 787), or DMTPV4 (SEQ ID NO: 788), or a RNA-null vector incorporated into a lipid-based transfection reagent. TaqManTm single-plex real-time quantitative polymerase chain reaction (RT-qPCR) was used to quantify levels of Grik2 knockdown in the GlutaNeurons in 384-well plates with three replicates post-transfection. GlutaNeurons were plated at a density of 17,500 cells/well (17.5k c/w). Grik2 mRNA expression was normalized to GAPDH signal. Transduction of AAV9 vectors encoding Grik2 antisense constructs was shown to significantly decrease Grik2 mRNA expression in GlutaNeurons after days for constructs containing G9 , GI, and DMTPV1-4 (FIG. 19). These findings demonstrate the effectiveness of dual-miRNA-encoding vectors targeting Grik2 in knocking down expression of Grik2 mRNA in cells endogenously expressing the Grik2 gene.

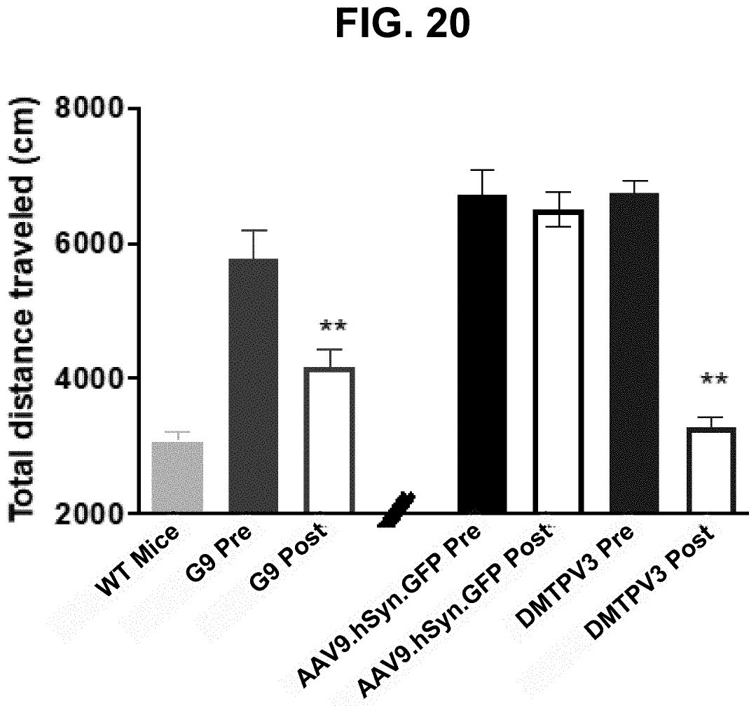

In a second set of experiments, spontaneous exploration behavior was tested with the open field test in mice treated with a vector encoding G9 (SEQ ID NO: 68), DMTPV3 (SEQ ID NO: 787), AAV9.hSyn.GFP, or historical control in untreated mice. Briefly, mice were placed into the center of a 50×50×50 cm blue polyvinyl chloride box for min, and the trajectories were recorded with a video camera connected to a tracking software EthoVision Color (Noldus, The Netherlands); the total distance covered by the mice during 10-minute exploration were analyzed. Mice treated with G9 and DMTPV3 showed a significant reduction in (p<0.01) in hyperlocomotor activity (FIG. 20).

Dose-dependence of the effect of DMTPV3 on hyperlocomotor activity in mice was tested across the four doses shown in Table 31, below.

TABLE 31
Details for miRNA constructs used in the study

| | | | Final virus |
|---|---|---|---|
| Vector # | Genome content | Volume | concentration |
| DMTPV3 | hSyn-GI-AmiR30a- | 1 μL × 4 | 3.6E+10 |
| | CAMKII-G9-miR124-3 | | |
| DMTPV3/10 | hSyn-GI-AmiR30a- | 1 μL × 4 | 3.6E+9 |
| | CAMKH-G9-miR124-3 | | |
| DMTPV3/100 | hSyn-GI-AmiR30a- | 1 μL × 4 | 3.6E+8 |
| | CAMKII-G9-miR124-3 | | |
| DMTPV3/1000 | hSyn-GI-AmiR30a- | 1 μL × 4 | 3.6E+7 |
| | CAMKII-G9-miR124-3 | | |
| Control | AAV9.hSyn.GFP | 1 μL × 4 | 3.6E+10 |

DMTPV3 and DMTPV3/10 vectors produced a significant reduction in hyperlocomotor activity following administration to mice (p<0.05; Mann-Whitney test; FIG. 21), thereby showing a dose-dependent effect of dual-antisense vectors on locomotor activity in mice.

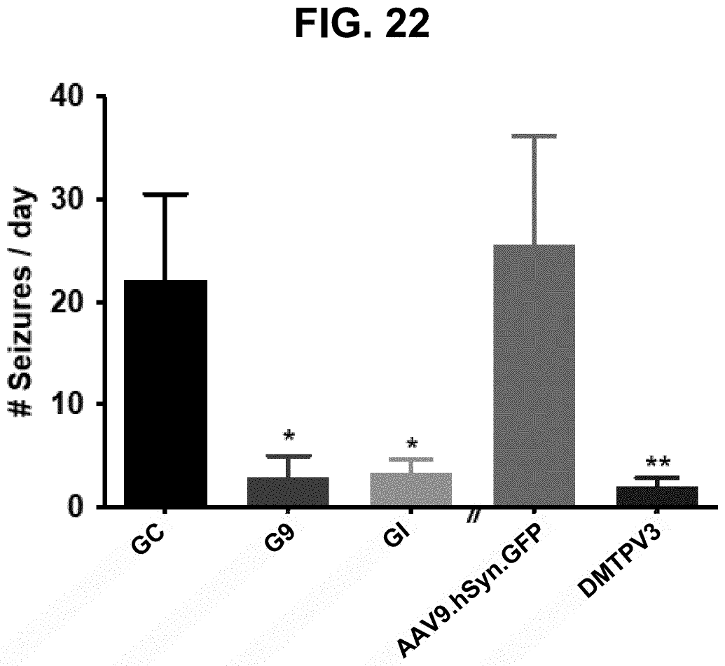

The anti-epileptogenic effects of vectors encoding single and dual miRNAs targeting Grik2 mRNA were also tested in pilocarpine-treated mice using vectors encoding single G9 (SEQ ID NO: 68) and GI (SEQ ID NO: 77) antisense sequences, dual-miRNA DMTPV3 (SEQ ID NO: 787) vector, or control vectors encoding a scrambled sequence GC (SEQ ID NO: 101) or AAV9.hSyn.GFP. Mice treated with G9, GI, and DMTPV3 showed a significant reduction in the number of seizures experienced per day, with DMTPV3 showing a significantly greater reduction than G9 or GI alone (FIG. 22). These findings demonstrate that dual-miRNA constructs targeting Grik2 mRNA show high potency in suppressing seizure activity in mice.

### Example10

### Knockdown of GluK2 Protein Expression Using Grik2 mRNA-Targeting AAV-Encoded Antisense Constructs in Organotypic Hippocampal Slices from Human Epileptic Patients

To assess the efficacy of AAV9 expression vectors encoding a Grik2 mRNA-targeting antisense oligonucleotide in knocking down GluK2 protein expression in human brain tissue, organotypic hippocampal slices were obtained from resected brain tissue of seven human TLE patients (H7, H8, H10, H13, 52, CBR15, and H14). Patient information is provided in Table 32, below.

TABLE 32
Patient information

| Patient code | Age, gender |
|---|---|
| H7 | Unknown |
| H8 | Unknown |
| H10 | 13, F |
| H13 | Unknown |
| H14 | Unknown |
| 52 | 47, M |
| CBR15 | 11, M |

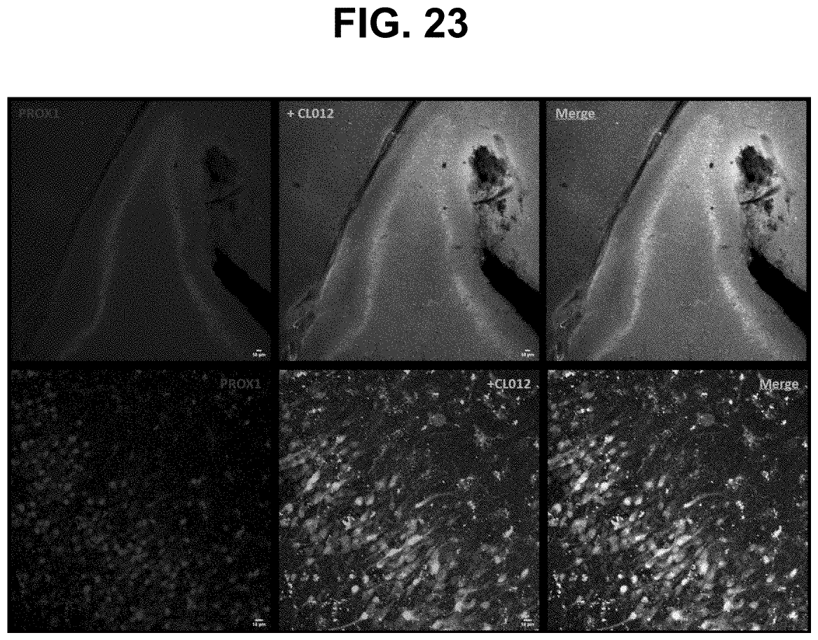

First, an organotypic hippocampal slice was obtained from a single human TLE patient, cultivated for 11-12 days in vitro in ACSF, and treated with a AAV9.GC(SEQ ID NO: 101).GFP vector. The slice was subsequently immunostained for Prox1 to identify dentate granule cells, GFP to identify cells transduced with the vector, and overlaid to determine the degree of overlap between the Prox1 and GFP signals. Over 50% of dentate granule cells were labeled with GFP and a substantial overlap between GFP and Prox1 was observed (FIG. 23). These results indicate that AAV9 vectors encoding RNA oligonucleotides can robustly transfect human dentate granule cells.

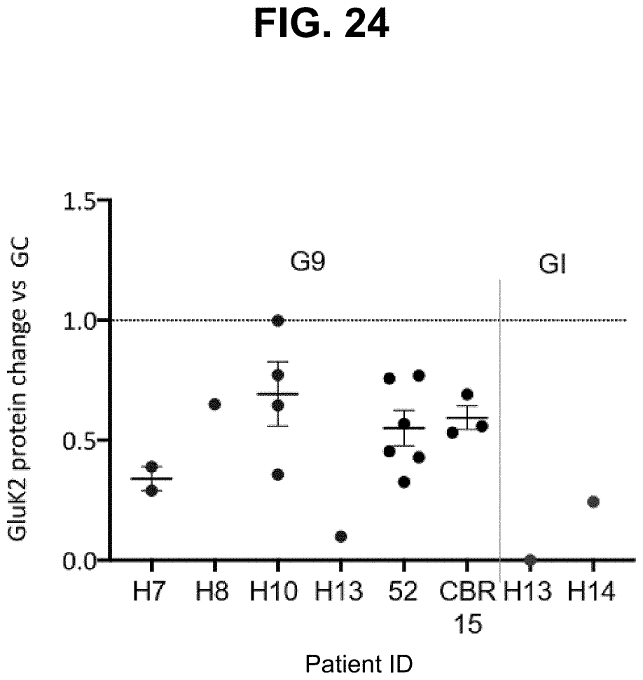

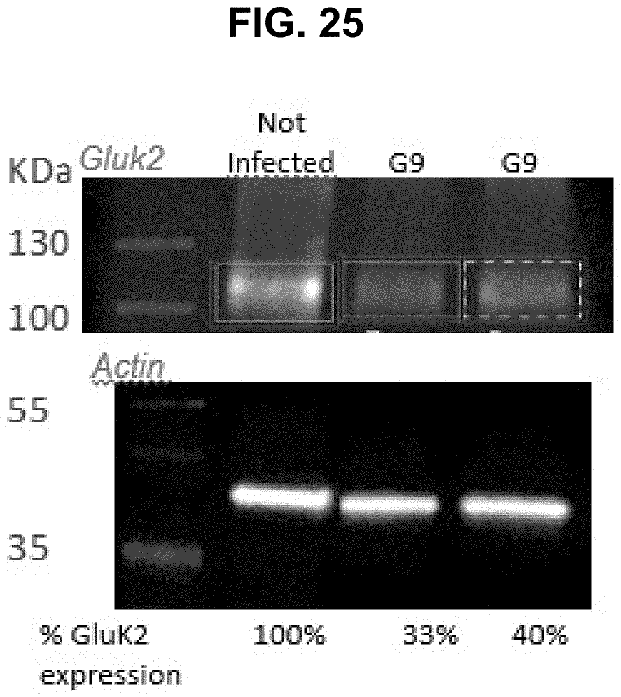

Human organotypic slices were treated with a Grik2 mRNA-targeting antisense construct G9 (SEQ ID NO: 68; 17 slices from six TLE patients) or antisense construct GI (SEQ ID NO: 77; two slices from two TLE patients). Western blots were performed on protein lysates from vector-treated slices. GluK2 protein levels were normalized to actin and quantified. Knockdown of GluK2 protein expression was observed in five out of five sets of human organotypic hippocampal slices treated with G9 (FIG. 24). An exemplary western blot performed on human slices treated with G9 is shown in FIG. 25, showing approximately a 40% reduction in GluK2 levels as compared to untreated slices.

### Example11

### In Vitro Efficacy of a Grik2 Mrna-Targeting Dual-Antisense Expression Construct in Human Hippocampal Slices

The efficacy of Grik2 mRNA-targeting, dual-miRNA AAV expression vectors was tested in vitro using organotypic hippocampal slices obtained from human subjects.

Ethics

For experiments using human cerebral tissue, all patients gave their written consent and protocols were approved by INSERM (No 2017-00031), and AP-HM (No M17-06) under the supervision of CRB TBM/AP-HM (No 271 KAI).

Cultured Human Hippocampal Slices from Epileptic Patients:

Human organotypic slices were prepared from surgical resections of the hippocampus from four patients (11-58 years old) diagnosed with drug-resistant TLE (Hopital de La Timone, Marseille, France). Tissue blocks were carefully transported from the hospital to the laboratory in a cold (2-5° C.), oxygenated modified artificial cerebrospinal fluid (mACSF); mACSF contained (in mM): 132 choline, 2.5 KCI, 1.25 NaH₂PO₄, 25 NaHCO_{3,} 7 MgCl₂, 0.5 CaCl₂, and 8 glucose) 300-400 pm thick slices were prepared under a biosafety cabinet using a vibratome (Leica VT1200S) in the same solution. After cutting, slices were recorded either the same day (acute slices) or cultured for several days in vitro before recordings (organotypic slices, see below). For culture, the slices were rinsed for minutes in oxygenated “washing medium” at room temperature (22° C.); washing medium contained Hanks Balanced Salt Solution (HBSS) completed with HEPES (mM). Glucose (17 mM) and antibiotics (1% Anti-Anti) were added for electrophysiological recordings. The organotypic slices were placed on individual cell cultures inserts (PICMORG50) in a 6-well plate (mm Transwell). One mL of culture medium was dropped in each well; the culture medium contained 50% MEM, 25% horse or human serum, 15% HBSS, 2% B27, 0.5% antibiotics, 11.8 mM glucose, and mM sucrose. Culture plates were maintained in an incubator at 37° C./5% CO₂. The culture medium was changed every two days and contained antibiotics for the first week (Anti-Anti). Electrophysiological recordings of organotypic slices were performed after 11 to DIV.

Viral Infection of Human Organotypic Slices

For infection, PBS medium containing AAV9 constructs was added directly on human organotypic slices at DIV1. Final virus titers were 1.8E+GC/mL for constructs GC (SEQ ID NO: 101) G9-S1 (SEQ ID NO: 775), and DMTPV3 (SEQ ID NO: 787), respectively. AAV9.hSyn.GFP vectors were used as a control for the G9-S1 and DMTPV3 experiments. Detailed information about the tested vectors is provided in Table 33, below.

TABLE 33
Details for miRNA single and dual constructs

| | | Final virus |
|---|---|---|
| Vector# | Genome content | concentration |
| G9-S1 (SEQ ID NO: 775) | hSyn.G9-S1 | 1.8E+10 |
| GC (SEQ ID NO: 101) | hSyn.GFP.GC-S1 | 1.8E+10 |
| DMTPV3 (SEQ ID NO: 787) | hSyn-GI-AmiR30a- | 1.8E+10 |
| | CAMKII-G9-miR124-3 | |

### Electrophysiological Recordings

Human organotypic slices were individually transferred to a recording chamber maintained at 30-32° C. and continuously perfused (2-3 mL/min) with oxygenated (95% 02 and 5% CO₂) ACSF (physiological condition), or hyperexcitable condition (ACSF containing 5 μM gabazine and 50 μM 4-AP). Local field potential (LFP) recordings were made with a monopolar Nichrome wire placed in the granule cell layer of the dentate gyrus. LFP was recorded with a DAM-80 amplifier (low filter, 0.1 Hz; highpass filter, 3 KHz; World Precision Instruments, Sarasota, FL); data were digitized (kHz) with a Digidata 1440A (Molecular Devices) to a computer, and acquired using Clampex 10.1 software (PClamp, Molecular Devices). Signals were analyzed off-line using Clampfit 9.2 (PClamp) and MiniAnalysis 6.0.1 (Synaptosoft, Decatur, Ga.). An epileptiform discharge (ED) consists of an increase in multi-unit firing with a LFP of duration >30 ms.

Statistics

All values are given as mean±SEM. Statistical analyses were performed using Graphpad Prism 7 (GraphPad Software, La Jolla, Calif.). For between-group comparisons, raw data were analyzed by a Mann-Whitney test. The level of significance was set at p<0.05.

Results

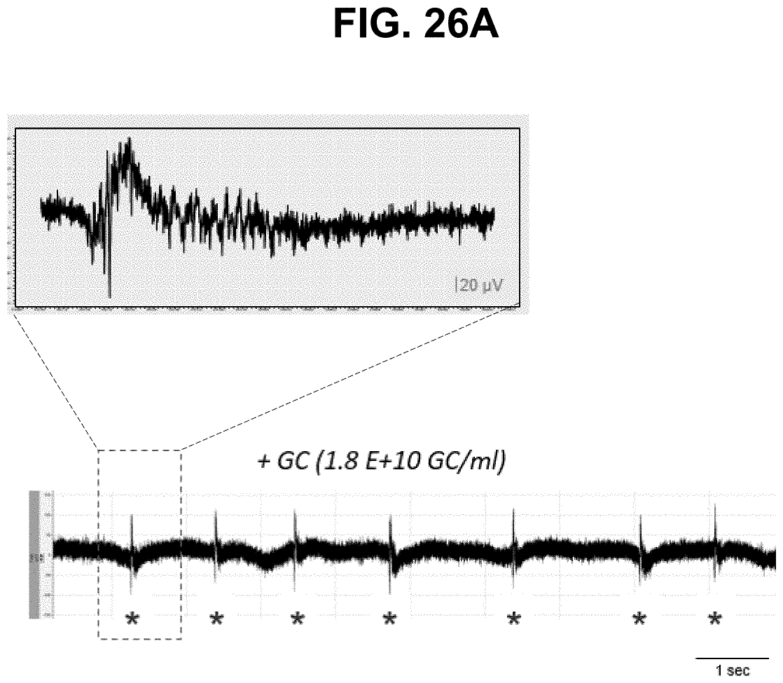

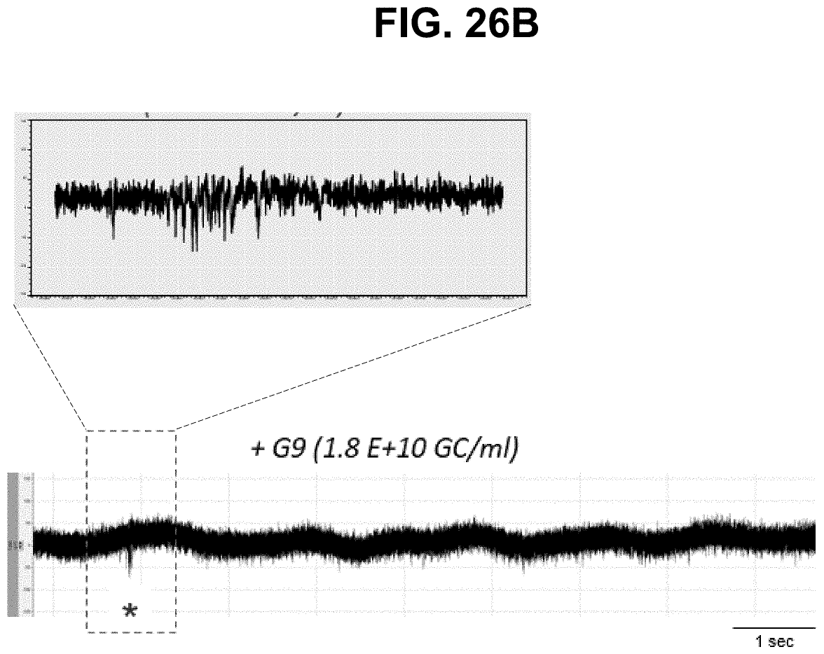

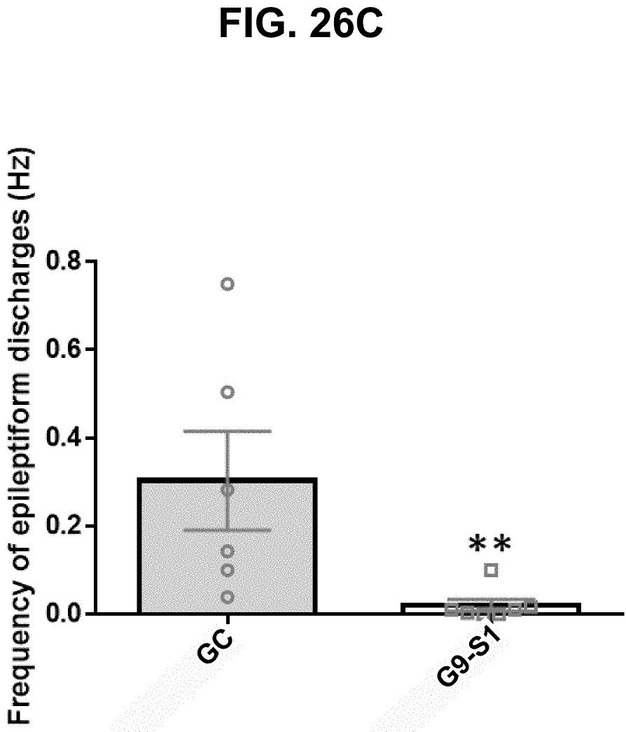

An AAV expression construct encoding a Grik2-targeting microRNA sequence, G9-S1 (SEQ ID NO: 775), a separate AAV construct encoding a scrambled control sequence, GC (SEQ ID NO: 101), and a third vector encoding a dual miRNA construct targeting Grik2, DMTPV3 (SEQ ID NO: 787) were tested in a physiological condition (see methods). Firstly, the functionality of the network to generate spontaneous EDs using a hyperexcitable solution (see methods) was evaluated; slices, which responded with ongoing spontaneous EDs were selected. These slices were then switched back to the standard physiological medium and washed for minutes. In control conditions (e.g., infection of the slices with the GC-encoding construct), the slices displayed numerous spontaneous recurrent EDs (0.3±0.11 Hz, n=6 slices; FIG. 26A). Remarkably, when slices were treated with G9-S1, we observed a suppression of EDs (by around 93%; 0.019 ±0.013 Hz, n=7 slices, p=0.0029, Mann-Whitney test; see FIGS. 26B-26C, Table 34).

TABLE 34
Epileptiform discharges-Mean ± SEM

| | GC | G9-S1 |
|---|---|---|
| Number of slices | 6 | 7 |
| Mean | 0.303 | 0.0198 |
| SEM | 0.1121 | 0.01355 |

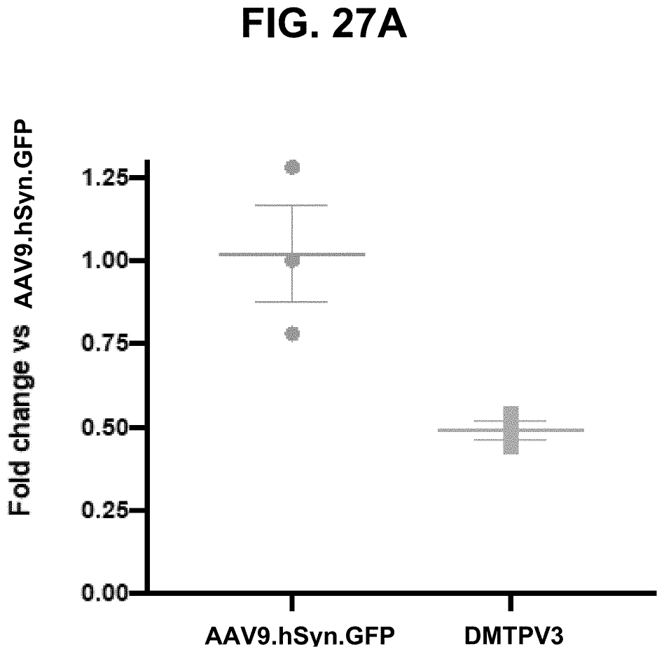

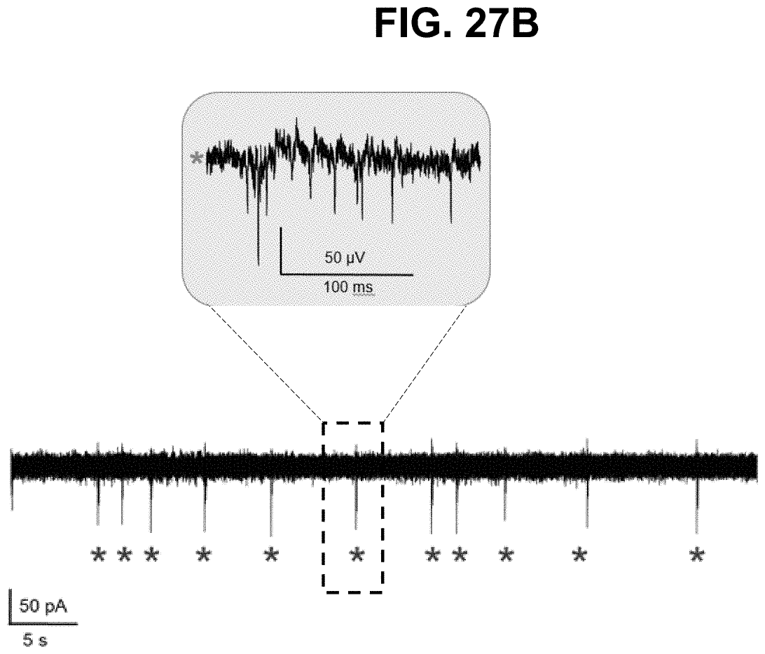

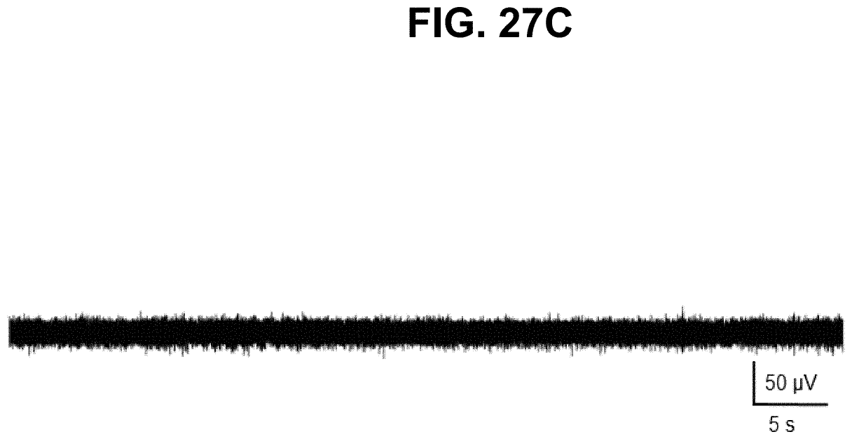

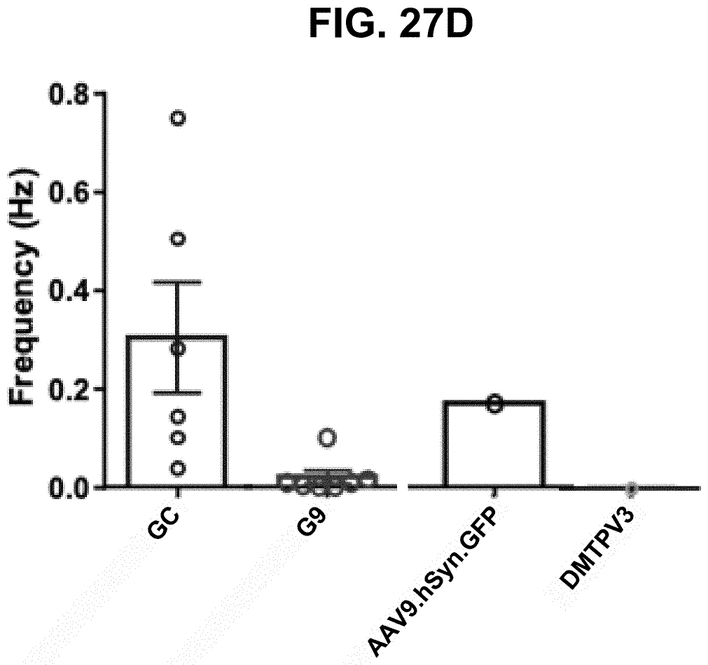

Moreover, treatment of human organotypic hippocampal slices with the dual antisense vector DMTPV3 produced a substantial reduction in levels of Grik2, as measured by qRT-PCR (FIG. 27A). When administered to slices under physiological conditions (e.g., in ACSF), DMTPV3 produced a profound reduction in epileptiform discharges (FIG. 27B) as compared to slices treated with AAV9.hSyn.GFP (FIGS. 27C-27D).

Therefore, these findings demonstrate that downregulation of GluK2 by AAV vectors encoding single and dual antisense sequences targeting Grik2 mRNA is an effective strategy for suppressing epileptiform activities in hippocampal tissue from patients with intractable TLE.

### Example12

### Analysis of Vector Multiplicity of Packaging

The impact of increasing vector length via inclusion of a stuffer sequence was evaluated. The stuffer (or filler) sequence incorporated into the vectors was a non-coding sequence selected for its apparent lack of cytotoxicity following gene delivery. Removal of regulatory elements involved in transcription initiation or completion within the non-coding sequence rendered the stuffer sequence inert with regards to transcriptional activity.

Vectors of various genome sizes were evaluated by electrophoresis using the TapeStation method. Analysis of the DNA molecules was performed according to the manufacturer's recommended protocols for High Sensitivity (HS) D5000 ScreenTape (Agilent #5067- 5592) using D5000 Reagents (Agilent #5067- 5593). TapeStation measures double-stranded (ds)DNA (rather than single-stranded (ss)DNA) and TapeStation evaluation relies on the annealing of two complementary ssAAV genomes to form an equivalent approximation of a dsAAV genome length in base pairs. Vectors encoding genomes having less than 50% the packaging capacity of AAV, exemplified in Table as SMSPV1 (SEQ ID NO: 25 818), SMSPV2 (SEQ ID NO: 820), and SMSPV3 (SEQ ID NO: 822)(SMSPV =Single MicroRNA Single Promoter Vector), each of which encodes a single miRNA cassette, has a genome length of 1.5-1.7 kb, and has the potential to package multiple (e.g., 2 or 3) genomes per capsid. As such, only 44%-61% of “full” capsids from SMSPV1, SMSPV2, and SMSPV3 preparations contain a single AAV genome. Addition of a stuffer sequence (e.g., SEQ ID NO: 815 and/or SEQ ID NO: 816) to each of these constructs (SMSPV4 (SEQ ID NO: 804), SMSPVS (SEQ ID NO: 806), and SMSPV6 (SEQ ID NO: 808), respectively), increases the percentage of capsids within each vector prep containing a single AAV genome to 72%-75%. Finally, addition of a stuffer sequence to a concatemerized genome encoding two distinct miRNAs (e.g., DMSPV1 (SEQ ID NO: 812); DMSPV =Dual MicroRNA Single Promoter Vector) results in 87% of capsids containing a single full-length AAV genome (see Table 35).

TABLE 35
Genome analysis of vectors with and
without stuffer sequence

| | | | | % Single |
|---|---|---|---|---|
| | SEQ | | Genome | full- |
| | ID | | Length | length |
| Construct | NO | Format | (kb) | genomes |
| SMSPV1 | 818 | Single miRNA | 1.5 | 59 |
| SMSPV4 | 804 | Single miRNA + stuffer | 3.1 | 75 |
| SMSPV2 | 820 | Single miRNA | 1.5 | 61 |
| SMSPV5 | 806 | Single miRNA + stuffer | 3.1 | 77 |
| SMSPV3 | 822 | Single miRNA | 1.7 | 44 |
| SMSPV6 | 808 | Single miRNA + stuffer | 3.3 | 72 |
| DMSPV1 | 812 | Two miRNA + stuffer | 3.45 | 87 |

### Example13

### In Vitro Knockdown of Gluk2 Expression in Mouse Cortical Neurons

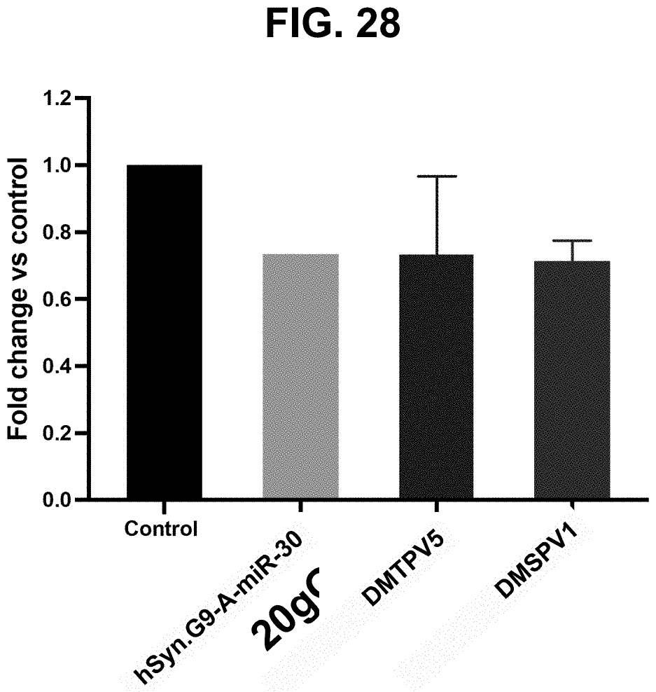

Dissociated cortical neurons from postnatal day 0 (P0)-P1 C57B16/J mouse were prepared and seeded in six-well plates at a concentration of 5.5E+cells per well. Two or three days after plating (days in vivo, DIV2-3), half of the medium was removed, and viral vectors were added with MOI 7.5E+4. At DIV 13, mouse neuronal cultures were lysed and the lysate was used for SDS-PAGE and immunoblotting. For immunostaining, the following antibodies were applied: rabbit anti-GluK2/3 (clone NL9 04-921; Merck-Millipore) diluted at 1:2000 and mouse anti- 3-actin (A5316; Sigma) diluted at 1:5000 were used as primary antibodies and an appropriate 800 nm fluorophore-conjugated secondary antibody produced in goat (IRDye 800 goat anti-mouse Li-COR 926-32210 or IRDye 800 goat anti-rabbit Li-COR 926-32211) diluted 1:15000 was used as secondary antibody. Target proteins were detected by measuring fluorescence at 800 nm on Li-COR. Analysis was performed with the Empiria studio software. For quantification, the intensity of the fluorescent signal of each lane was normalized by 3-actin expression and then by the control condition. Under these experimental conditions, expression constructs DMTPV8 (SEQ ID NO: 813) and DMSPV1 (SEQ ID NO: 812) presented a reduction in GluK2 expression of 73±17% and 71 ±4% vs. control vector respectively (mean ±S.E.M.; see FIG. 28).

### Example14

### In Vivo Knockdown of Gluk2 in a Mouse Model of Temporal Lobe Epilepsy

Experiments were performed using 6-9 weeks old Swiss male mice. Mice (30-g) were given scopolamine (1 mg/kg) subcutaneously (s.c.) minutes prior to intra-peritoneal administration of pilocarpine (300-350 mg/kg). Caffeine (mg/kg) was administered minutes after the first seizure. Diazepam (mg/kg) was administered one hour after onset of status epilepticus. For AAV administration, epileptic mice (>2 months after status epilepticus) were anesthetized under isoflurane anaesthesia (5% for induction and 2% for maintenance, under 100% 02). In the presence of local administration of lidocaine (2%), four holes were drilled to bilaterally inject AAV into the dorsal and ventral dentate gyrus of the hippocampus (AP -1.8 mm, ML ±1 mm, DV-2 mm, and AP-3.3 mm, ML±2.3 mm, DV-2.5 mm). For each injection, a Hamilton syringe was used for AAV delivery. After a 5-minute delay to allow the brain tissue to slide over the cannula, a volume =1.0 μL/injection site x 4 injection sites of a solution containing AAV was slowly infused (rate: 0.2 μL/minute). The locomotion of epileptic mice (>2 months after status epilepticus) was evaluated one week before and two weeks after AAV injection. The locomotion of non-epileptic mice (wild-type Swiss male mice, 18-21 weeks old) were also evaluated. Mice were transferred to the behaviour analysis room one day prior experiments for habituation to the environment; the mice were kept at room temperature (20-22° C.) in a 9:00—18:00 light/dark cycle with ad libitum access to food and water. All materials that have been in contact with the animal tested were washed with acetic acid thereafter in order to prevent olfactory cues. First, spontaneous exploration behaviour was tested with the open field test described above. Briefly, the mice were placed into the center of a 50×50×50 cm blue polyvinyl chloride box for min, and the trajectories were recorded with a video camera connected to a tracking software EthoVision Color (Noldus, The Netherlands); the speed and the total distance covered by the mice during 10-minute spontaneous exploration were analysed.

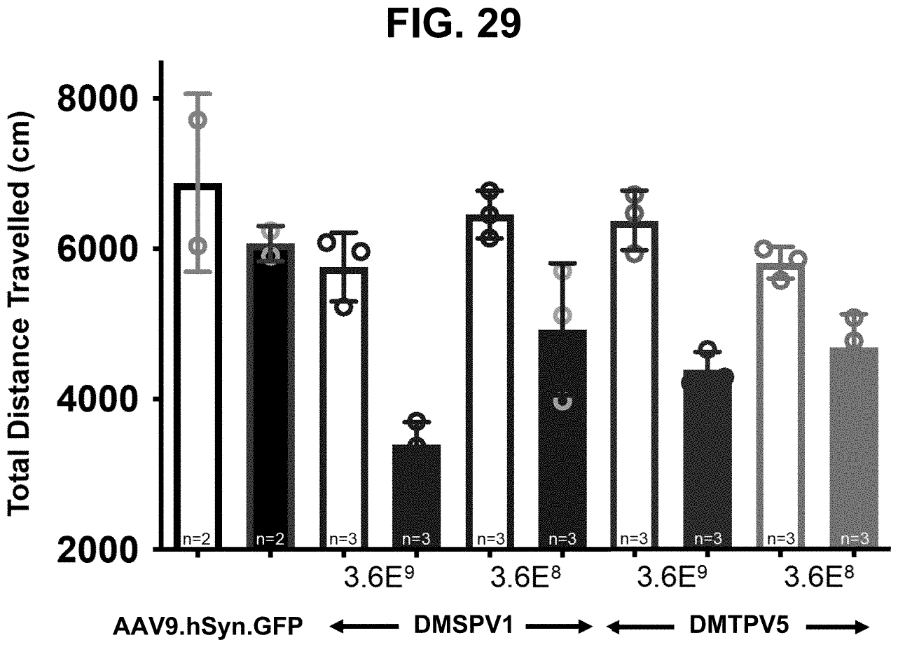

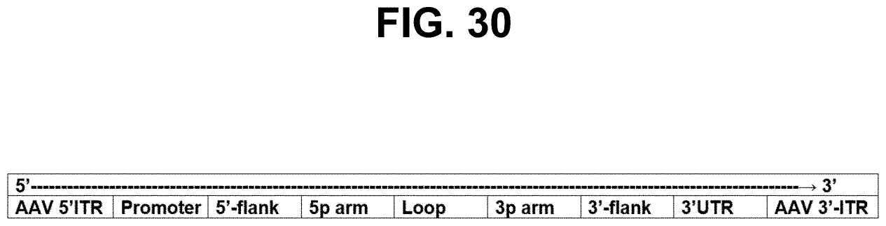

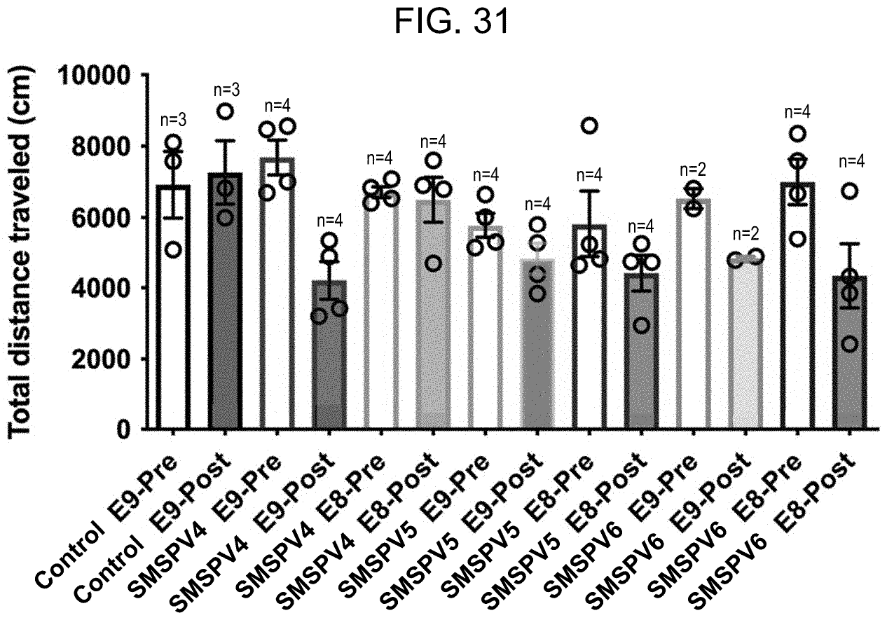

Treatment of mice with DMTPV8 and DMSPV1 led to a decrease in hyper-locomotion, which is a proxy for epileptogenic behavior. This decrease in hyper-locomotion was dose-dependent. While both doses showed efficacy for DMTPV8 and DMSPV1, 3.6xE+9 was more effective than 3.6xE+8. DMSPV1 at the high dose of 3.6xE+9 had the strongest effect of all conditions tested (FIG. 29).

In a separate set of experiments, mice were treated with vector constructs SMSPV4 (SEQ ID NO: 804), SMSPVS (SEQ ID NO: 806), or SMSPV6 (SEQ ID NO: 808) and locomotion was tested in the same way as described above. Treatment of mice with these constructs led to a decrease in hyperlocomotion (a proxy for epileptogenic behavior), whereas treatment with a control vector (CV) did not have any effect (FIG. 31). The CV was designed to mimic an AAV capsid that contains a genome that does not produce mRNA or protein, but instead contains, from 5′ ITR to 3′ ITR: RBG polyA (SEQ ID NO: 751), CpG-depleted chicken beta actin intron (SEQ ID NO: 824), and a non-coding stuffer sequence of SEQ ID NO: 823. The upstream RBG polyA sequence allows accurate ddPCR titer comparison with other AAV vectors and eliminates any potential transcription from the 5′ ITR. The decrease in hyperlocomotion following treatment with the anti-Grik2 constructs was dose-dependent. While both doses, 3.6xE+9 and 3.6xE+8, showed efficacy for SMSPV6, the 3.6xE+9 dose was more effective than 3.6xE+8 for SMSPV4. For SMSPVS, both 3.6xE+9 and 3.6xE+8 dosages, showed similar efficacy. SMSPV4 had the strongest effect at the high dose of 3.6xE+9 of all conditions tested, while SMSPV4 at the low dose of 3.6xE+8 had no effect on hyperlocomotion (FIG. 31).

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

### REFERENCES

Throughout this application, various references describe the state of the art to which this disclosure pertains. The disclosures of these references are hereby incorporated by reference into the present disclosure:

Bahn S., Volk B., Wisden W. (1994). Kainate receptor gene expression in the developing rat brain. J. Neurosci. 14 5525-5547. 10.1523/JNEUROSCI.14-09-05525.1994.

Boudreau Ryan L., Rodriguez-Lehr& Edgardo, Davidson Beverly L., RNAi medicine for the brain: progresses and challenges, Human Molecular Genetics, Volume 20, Issue R1, 15 April 2011, Pages R21—R27

Bouvier G., Larsen R. S., Rodriguez-Moreno A., Paulsen 0., Sjostrom P. J. (2018). Towards resolving the presynaptic NMDA receptor debate. Curr. Opin. Neurobiol. 51 1-7. 10.1016/j.conb.2017.12.020

Crepel V, Mulle C (2015) Physiopathology of kainate receptors in epilepsy. Curr Opin Pharmacol 20:83-88; doi: 10.1016/j.coph.2014.11.012. Epub 2014 Dec 13.

Englot, D J., et al (2013) Seizure outcomes after resective surgery for extra—temporal lobe epilepsy in pediatric patients: A systematic review. J. Neurosurgery. 12(2):97-201

Fritsch B., Reis J., Gasior M., Kaminski R. M., Michael A., Rogawski M. A. (2014). Role of GluK1 kainate receptors in seizures, epileptic discharges, and epileptogenesis. J. Neurosci. 34 5765-5775. 10.1523/JNEUROSC1.5307-13.2014

Gabriel S, Njunting M, Pomper J K, Merschhemke M, Sanabria ERG, Eilers A, Kivi A, Zeller M, Meencke H-J, Cavalheiro E a, Heinemann U, Lehmann T-N (2004) Stimulus and potassium-induced epileptiform activity in the human dentate gyrus from patients with and without hippocampal sclerosis. J Neurosci 24:10416-10430.

Gruber A., Lorenz R., Bernhart S. H., NeubOck R., Hofacker I.L (2008). The Vienna RNA Websuite. Nucleic Acids Research. 36:W70-4

Hardy J. (2010). Genetic analysis of pathways to Parkinson disease. Neuron, 68(2), 201-206. doi:10.1016/j.neuron.2010.10.014

Melyan Z., Lancaster B., Wheal H. V. (2004). Metabotropic regulation of intrinsic excitability by synaptic activation of kainate receptors. J. Neurosci. 24 4530-4534. 10.1523/JNEUROSCI.5356-03.2004

Melyan Z., Wheal H. V., Lancaster B. (2002). Metabotropic-mediated kainate receptor regulation of isAHP and excitability in pyramidal cells. Neuron 34 107-114. 10.1016/S0896-6273(02)00624-4

Mulle C., Sailer A., Perez-Otario I., Dickinson-Anson H., Castillo P. E., Bureau I., et al. (1998). Altered synaptic physiology and reduced susceptibility to kainate-induced seizures in GluR6-deficient mice. Nature 392 601-605.

Peret A, Christie L a., Ouedraogo D W, Gorlewicz A, Epsztein J, Mulle C, Crepel V (2014) Contribution of Aberrant GluK2-Containing Kainate Receptors to Chronic Seizures in Temporal Lobe Epilepsy. Cell Rep8:347-354.

Reiner A, Arant R J, and Isacoff E Y (2012) Assembly Stoichiometry of the GluK2/GluKKainate Receptor Complex. Cell Rep1:234-240.

Represa A, Le Gall La Salle G, Ben-Ari Y (1989a) Hippocampal plasticity in the kindling model of epilepsy in rats. Neurosci Lett 99:345-350.

Represa A, Robain 0, Tremblay E, Ben-Ari Y (1989b) Hippocampal plasticity in childhood epilepsy. Neurosci Lett 99:351-355.

Rodriguez-Moreno A., Herreras 0., Lerma J. (1997). Kainate receptors presynaptically downregulate GABAergic inhibition in the rat hippocampus. Neuron 19 893-901. 10.1016/S0896-6273(00)80970-8.

Rodriguez-Moreno A., Sihra T. S. (2007a). Kainate receptors with a metabotropic modus operandi. Trends Neurosci. 30 630-637.

Rodriguez-Moreno A., Sihra T. S. (2007b). Metabotropic actions of kainate receptors in the CNS. J. Neurochem. 103 2121-2135.

Sapru Mohan K., Yates Jonathan W., Hogan Shea, Jiang Lixin, Halter Jeremy, Bohn Martha C. (2006). Silencing of human a-synuclein in vitro and in rat brain using lentiviral-mediated RNAi. Neurology.198:382-390

Smolders I., Bortolotto Z. A., Clarke V. R., Warre R., Khan G. M., O′Neill M. J., et al. (2002). Antagonists of GLU(K5)-containing kainate receptors prevent pilocarpine-induced limbic seizures. Nat. Neurosci. 5 796-804. 10.1038/nn88

Sutula T, Cascino G, Cavazos J, Parada I, Ramirez L (1989) Mossy fiber synaptic reorganization in the epileptic human temporal lobe. Ann Neurol 26:321-330.

Tauck D L, Nadler J V (1985) Evidence of functional mossy fiber sprouting in hippocampal formation of kainic acid-treated rats. J Neurosci 5:1016-1022

Valbuena S., Lerma J. (2016). Non-canonical signaling, the hidden life of ligand-gated ion channels. Neuron 92 316-329. 10.1016/j.neuron.2016.10.016

Wang L., Bai J., and Hu Y. (2007) Identification of the RA Response Element and Transcriptional Silencer in Human alphaCaMKII Promoter. Mol. Biol. Rep. 35(1):37-44

Zinn, E., Pacouret, S., Khaychuk, V., Turunen, H. T., Carvalho, L. S., Andres-Mateos, E., . . . Vandenberghe, L. H. (2015). In Silico Reconstruction of the Viral Evolutionary Lineage Yields a Potent Gene Therapy Vector. Cell reports, 12(6), 1056-1068. doi:10.1016/j.celrep.2015.07.019

1. An isolated polynucleotide having a length of no more than 800 nucleotides that specifically hybridizes within a single-stranded region of a Grik2 mRNA, wherein the hybridized polynucleotide has a Target Opening Energy of less than 18 kcal/mol, and wherein:
(a) the polynucleotide does not include the nucleic acid sequence of any one of SEQ ID NOs: 772-774;
(b) the polynucleotide comprises the nucleic acid sequence of SEQ ID NO: 68 or SEQ ID NO: 68 and SEQ ID NO: 649; or
(c) the polynucleotide does not have a Total Opening energy that is between 5.53 kcal/mol and 5.55 kcal/mol.

2. An isolated RNA polynucleotide having a length of no more than 23 nucleotides that specifically hybridizes within a single-stranded region of a Grik2 mRNA, wherein the hybridized polynucleotide has a Total Opening Energy of less than 18 kcal/mol, wherein the polynucleotide does not include the nucleic acid sequence of any one of SEQ ID NOs: 772-774.

3. The polynucleotide of claim 1 or 2, wherein the polynucleotide has a Total Opening energy that is greater than 5.4 kcal/mol or less than 5.4 kcal/mol.

4. The polynucleotide of any one of claims 1-3, wherein the hybridized polynucleotide has an Energy of Duplex Formation that is greater than −kcal/mol.

5. The polynucleotide of any one of claim 1-4, wherein the hybridized polynucleotide has an Total Energy of Binding of greater than −24 kcal/mol.

6. The polynucleotide of any one of claims 1-5, wherein the hybridized polynucleotide has a GC content that is less than 50%.

7. The polynucleotide of any one of claims 1-6, wherein the single-stranded region of the Grik2 mRNA is selected from the group consisting of Loop regions 1-14.

8. The polynucleotide of any one of claims 1-7, wherein the polynucleotide specifically hybridizes within:
(a) a Loop1 region of the Grik2 mRNA;
(b) a Loop2 region of the Grik2 mRNA;
(c) a Loop3 region of the Grik2 mRNA;
(d) a Loop4 region of the Grik2 mRNA;
(e) a Loop region of the Grik2 mRNA;
(f) a Loop6 region of the Grik2 mRNA;
(g) a Loop7 region of the Grik2 mRNA;
(h) a Loop8 region of the Grik2 mRNA;
(i) a Loop9 region of the Grik2 mRNA;
(j) a Loop region of the Grik2 mRNA;
(k) a Loop11 region of the Grik2 mRNA;
(I) a Loop12 region of the Grik2 mRNA;
(m) a Loop13 region of the Grik2 mRNA; or
(n) a Loop14 region of the Grik2 mRNA.

9. The polynucleotide of claim 7 or claim 8, wherein:
(a) the Loop1 region is encoded by a nucleic acid sequence having at least 85%, 90%, 92%, 95%, 97%, 99%, or 100% sequence identity over at least contiguous nucleotides of SEQ ID NO: 145;
(b) the Loop2 region is encoded by a nucleic acid sequence having at least 85%, 90%, 92%, 95%, 97%, 99%, or 100% sequence identity over at least contiguous nucleotides of SEQ ID NO: 146;
(c) the Loop3 region is encoded by a nucleic acid sequence having at least 85%, 90%, 92%, 95%, 97%, 99%, or 100% sequence identity over at least contiguous nucleotides of SEQ ID NO: 147;
(d) the Loop4 region is encoded by a nucleic acid sequence having at least 85%, 90%, 92%, 95%, 97%, 99%, or 100% sequence identity over at least contiguous nucleotides of SEQ ID NO: 148;
(e) the Loop region is encoded by a nucleic acid sequence having at least 85%, 90%, 92%, 95%, 97%, 99%, or 100% sequence identity over at least contiguous nucleotides of SEQ ID NO: 149;
(f) the Loop6 region is encoded by a nucleic acid sequence having at least 85%, 90%, 92%, 95%, 97%, 99%, or 100% sequence identity over at least contiguous nucleotides of SEQ ID NO: 150;
(g) the Loop7 region is encoded by a nucleic acid sequence having at least 85%, 90%, 92%, 95%, 97%, 99%, or 100% sequence identity over at least contiguous nucleotides of SEQ ID NO: 151;
(h) the Loop8 region is encoded by a nucleic acid sequence having at least 85%, 90%, 92%, 95%, 97%, 99%, or 100% sequence identity over at least contiguous nucleotides of SEQ ID NO: 152;
(i) the Loop9 region is encoded by a nucleic acid sequence having at least 85%, 90%, 92%, 95%, 97%, 99%, or 100% sequence identity over at least contiguous nucleotides of SEQ ID NO: 153;
(j) the Loop region is encoded by a nucleic acid sequence having at least 85%, 90%, 92%, 95%, 97%, 99%, or 100% sequence identity over at least contiguous nucleotides of SEQ ID NO: 154;
(k) the Loop11 region is encoded by a nucleic acid sequence having at least 85%, 90%, 92%, 95%, 97%, 99%, or 100% sequence identity over at least contiguous nucleotides of SEQ ID NO: 155;
(l) the Loop12 region is encoded by a nucleic acid sequence having at least 85%, 90%, 92%, 95%, 97%, 99%, or 100% sequence identity over at least contiguous nucleotides of SEQ ID NO: 156;
(m) the Loop13 region is encoded by a nucleic acid sequence having at least 85%, 90%, 92%, 95%, 97%, 99%, or 100% sequence identity over at least contiguous nucleotides of SEQ ID NO: 157; and/or
(n) the Loop14 region is encoded by a nucleic acid sequence having at least 85%, 90%, 92%, 95%, 97%, 99%, or 100% sequence identity over at least contiguous nucleotides of SEQ ID NO: 158.

10. The polynucleotide of claim 9, wherein the sequence identity is determined over at least contiguous nucleotides of any one of SEQ ID NOs: 145-158.

11. The polynucleotide of claim 10, wherein the sequence identity is determined over at least contiguous nucleotides of any one of SEQ ID NOs: 145-158.

12. The polynucleotide of claim 11, wherein sequence identity is determined over at least 60 contiguous nucleotides of any one of SEQ ID NOs: 145-158.

13. The polynucleotide of claim 12, wherein sequence identity is determined over the full length of any one of SEQ ID NOs: 145-158.

14. The polynucleotide of any one of claims 1-13, wherein the polynucleotide comprises:
(a) a nucleic acid sequence having at least 85%, 90%, 92%, 95%, 97%, 99%, or 100% sequence identity over at least contiguous nucleotides of SEQ ID NO: 1;
(b) a nucleic acid sequence having at least 85%, 90%, 92%, 95%, 97%, 99%, or 100% sequence identity over at least contiguous nucleotides of SEQ ID NO: 4;
(c) (i) a nucleic acid sequence having at least 85%, 90%, 92%, 95%, 97%, 99%, or 100% sequence identity over at least contiguous nucleotides of SEQ ID NO: 5; or
(ii) a nucleic acid sequence having at least 85%,90%, 92%, 95%, 97%, 99%, or 100% sequence identity over at least contiguous nucleotides of SEQ ID NO: 6;
(d) a nucleic acid sequence having at least 85%, 90%, 92%, 95%, 97%, 99%, or 100% sequence identity over at least contiguous nucleotides of SEQ ID NO: 7;
(e) a nucleic acid sequence having at least 85%, 90%, 92%, 95%, 97%, 99%, or 100% sequence identity over at least contiguous nucleotides of SEQ ID NO: 96;
(f) (i) a nucleic acid sequence having at least 85%, 90%, 92%, 95%, 97%, 99%, or 100% sequence identity over at least contiguous nucleotides of SEQ ID NO: 8;
(ii) a nucleic acid sequence having at least 85%, 90%, 92%, 95%, 97%, 99%, or 100% sequence identity over at least contiguous nucleotides of SEQ ID NO: 98; or
(iii) a nucleic acid sequence having at least 85%, 90%, 92%, 95%, 97%, 99%, or 100% sequence identity over at least contiguous nucleotides of SEQ ID NO: 99;
(g) a nucleic acid sequence having at least 85%, 90%, 92%, 95%, 97%, 99%, or 100% sequence identity over at least contiguous nucleotides of SEQ ID NO: 9;
(h) a nucleic acid sequence having at least 85%, 90%, 92%, 95%, 97%, 99%, or 100% sequence identity over at least contiguous nucleotides of SEQ ID NO: 63;
(i) a nucleic acid sequence having at least 85%, 90%, 92%, 95%, 97%, 99%, or 100% sequence identity over at least contiguous nucleotides of SEQ ID NO: 10; or
(j) a nucleic acid sequence having at least 85%, 90%, 92%, 95%, 97%, 99%, or 100% sequence identity over at least contiguous nucleotides of SEQ ID NO: 11.

15. The polynucleotide of claim 14, wherein sequence identity is determined over at least contiguous nucleotides of any one of SEQ ID NOs: 1, 4-11, 63, 96, 98, or 99.

16. The polynucleotide of claim 15, wherein sequence identity is determined over at least contiguous nucleotides of any one of SEQ ID NOs: 1, 4-11, 63, 96, 98, or 99.

17. The polynucleotide of claim 16, wherein sequence identity is determined over at least contiguous nucleotides of any one of SEQ ID NOs: 1, 4-11, 63, 96, 98, or 99.

18. The polynucleotide of claim 17, wherein sequence identity is determined over the full length of any one of SEQ ID NOs: 1, 4-11, 63, 96, 98, or 99.

19. The polynucleotide of any one of claims 1-18, wherein the polynucleotide comprises a duplex structure formed by the polynucleotide and the single-stranded region of the Grik2 mRNA, wherein the duplex structure comprises at least one mismatch between the nucleotides of the polynucleotide and nucleotides of the single-stranded region of the Grik2 mRNA.

20. The polynucleotide of claim 19, wherein the single-stranded region of the Grik2 mRNA is selected from the group consisting of Loop regions 1-14.

21. The polynucleotide of any one of claims 1-20, wherein the Total Opening Energy, Energy of Duplex Formation, Total Energy of Binding, and/or GC content is calculated over 23 to 79 nucleotides.

22. The polynucleotide of any one of claims 1-7, wherein the single-stranded region of the Grik2 mRNA is selected from the group consisting of Unpaired regions 1-5.

23. The polynucleotide of claim 22, wherein the polynucleotide specifically hybridizes within:
(a) a Unpaired region 1 of the Grik2 mRNA;
(b) a Unpaired region 2 of the Grik2 mRNA;
(c) a Unpaired region 3 of the Grik2 mRNA;
(d) a Unpaired region 4 of the Grik2 mRNA; or
(e) a Unpaired region of the Grik2 mRNA.

24. The polynucleotide of claim 22 or 23, wherein:
(a) the Unpaired region 1 is encoded by a nucleic acid sequence having at least 85%, 90%, 92%, 95%, 97%, 99%, or 100% sequence identity over at least contiguous nucleotides of SEQ ID NO: 159;
(b) the Unpaired region 2 is encoded by a nucleic acid sequence having at least 85%, 90%, 92%, 95%, 97%, 99%, or 100% sequence identity over at least contiguous nucleotides of SEQ ID NO: 160;
(c) the Unpaired region 3 is encoded by a nucleic acid sequence having at least 85%, 90%, 92%, 95%, 97%, 99%, or 100% sequence identity over at least contiguous nucleotides of SEQ ID NO: 161;
(d) the Unpaired region 4 is encoded by a nucleic acid sequence having at least 85%, 90%, 92%, 95%, 97%, 99%, or 100% sequence identity over at least contiguous nucleotides of SEQ ID NO: 162; and/or
(e) the Unpaired region is encoded by a nucleic acid sequence having at least 85%, 90%, 92%, 95%, 97%, 99%, or 100% sequence identity over at least contiguous nucleotides of SEQ ID NO: 163.

25. The polynucleotide of claim 24 wherein sequence identity is determined over at least contiguous nucleotides of any one of SEQ ID NOs: 159-163.

26. The polynucleotide of claim 25, wherein sequence identity is determined over at least contiguous nucleotides of any one of SEQ ID NOs: 159-163.

27. The polynucleotide of claim 26, wherein sequence identity is determined over at least 60 contiguous nucleotides of any one of SEQ ID NOs: 159-163.

28. The polynucleotide of claim 27, wherein sequence identity is determined over the full length of any one of SEQ ID NOs: 159-163.

29. The polynucleotide of any one of claims 22-28, wherein the polynucleotide comprises:
(a) (i) a nucleic acid sequence having at least 85%, 90%, 92%, 95%, 97%, 99%, or 100% sequence identity over at least contiguous nucleotides of SEQ ID NO: 13;
(ii) a nucleic acid sequence having at least 85%, 90%, 92%, 95%, 97%, 99%, or 100% sequence identity over at least contiguous nucleotides of SEQ ID NO: 14;
(iii) a nucleic acid sequence having at least 85%, 90%, 92%, 95%, 97%, 99%, or 100% sequence identity over at least contiguous nucleotides of SEQ ID NO: 72; or
(iv) a nucleic acid sequence having at least 85%, 90%, 92%, 95%, 97%, 99%, or 100% sequence identity over at least contiguous nucleotides of SEQ ID NO: 73;
(b) a nucleic acid sequence having at least 85%, 90%, 92%, 95%, 97%, 99%, or 100% sequence identity over at least contiguous nucleotides of SEQ ID NO: 15; or
(c) a nucleic acid sequence having at least 85%, 90%, 92%, 95%, 97%, 99%, or 100% sequence identity over at least contiguous nucleotides of SEQ ID NO: 16.

30. The polynucleotide of claim 29, wherein sequence identity is determined over at least contiguous nucleotides of any one of SEQ ID NOs: 13-16, 72 or 73.

31. The polynucleotide of claim 30, wherein sequence identity is determined over at least contiguous nucleotides of any one of SEQ ID NOs: 13-16, 72 or 73.

32. The polynucleotide of claim 31, wherein sequence identity is determined over at least contiguous nucleotides of any one of SEQ ID NOs: 13-16, 72 or 73.

33. The polynucleotide of claim 32, wherein sequence identity is determined over the full length of any one of SEQ ID NOs: 13-16, 72 or 73.

34. The polynucleotide of any one of claims 29-33, wherein sequence identity is determined over no more than contiguous nucleotides of any one of SEQ ID NOs: 13-16, 72 or 73.

35. The polynucleotide of claim 34, wherein sequence identity is determined over no more than contiguous nucleotides of any one of SEQ ID NOs: 13-16, 72 or 73.

36. The polynucleotide of any one of claims 22-35, wherein the polynucleotide comprises a duplex structure formed by the polynucleotide and the single-stranded region of the Grik2 mRNA, wherein the duplex structure comprises at least one mismatch between the nucleotides of the polynucleotide and nucleotides of the single-stranded region of the Grik2 mRNA.

37. The polynucleotide of any one of claims 22-36, wherein the average positional entropy is calculated over 23 to 79 nucleotides.

38. The polynucleotide of any one of claims 1-37, wherein the polynucleotide hybridizes to a coding sequence of the Grik2 mRNA.

39. The polynucleotide of claim 38, wherein the polynucleotide hybridizes to:
(a) a region within exon 1 of the Grik2 mRNA;
(b) a region within exon 2 of the Grik2 mRNA;
(c) a region within exon 3 of the Grik2 mRNA;
(d) a region within exon 4 of the Grik2 mRNA;
(e) a region within exon of the Grik2 mRNA;
(f) a region within exon 6 of the Grik2 mRNA;
(g) a region within exon 7 of the Grik2 mRNA;
(h) a region within exon 8 of the Grik2 mRNA;
(i) a region within exon 9 of the Grik2 mRNA;
(j) a region within exon of the Grik2 mRNA;
(k) a region within exon 11 of the Grik2 mRNA;
(l) a region within exon 12 of the Grik2 mRNA;
(m) a region within exon 13 of the Grik2 mRNA;
(n) a region within exon 14 of the Grik2 mRNA;
(o) a region within exon of the Grik2 mRNA; and/or
(p) a region within exon 16 of the Grik2 mRNA.

40. The polynucleotide of claim 39, wherein:
(a) exon 1 of the Grik2 mRNA is encoded by a nucleic acid sequence having at least 85%, 90%, 92%, 95%, 97%, 99%, or 100% sequence identity over at least contiguous nucleotides of SEQ ID NO: 129;
(b) exon 2 of the Grik2 mRNA is encoded by a nucleic acid sequence having at least 85%, 90%, 92%, 95%, 97%, 99%, or 100% sequence identity over at least contiguous nucleotides of SEQ ID NO: 130;
(c) exon 3 of the Grik2 mRNA is encoded by a nucleic acid sequence having at least 85%, 90%, 92%, 95%, 97%, 99%, or 100% sequence identity over at least contiguous nucleotides of SEQ ID NO: 131;
(d) exon 4 of the Grik2 mRNA is encoded by a nucleic acid sequence having at least 85%, 90%, 92%, 95%, 97%, 99%, or 100% sequence identity over at least contiguous nucleotides of SEQ ID NO: 132;
(e) exon of the Grik2 mRNA is encoded by a nucleic acid sequence having at least 85%, 90%, 92%, 95%, 97%, 99%, or 100% sequence identity over at least contiguous nucleotides of SEQ ID NO: 133;
(f) exon 6 of the Grik2 mRNA is encoded by a nucleic acid sequence having at least 85%, 90%, 92%, 95%, 97%, 99%, or 100% sequence identity over at least contiguous nucleotides of SEQ ID NO: 134;
(g) exon 7 of the Grik2 mRNA is encoded by a nucleic acid sequence having at least 85%, 90%, 92%, 95%, 97%, 99%, or 100% sequence identity over at least contiguous nucleotides of SEQ ID NO: 135;
(h) exon 8 of the Grik2 mRNA is encoded by a nucleic acid sequence having at least 85%, 90%, 92%, 95%, 97%, 99%, or 100% sequence identity over at least contiguous nucleotides of SEQ ID NO: 136;
(i) exon 9 of the Grik2 mRNA is encoded by a nucleic acid sequence having at least 85%, 90%, 92%, 95%, 97%, 99%, or 100% sequence identity over at least contiguous nucleotides of SEQ ID NO: 137;
(j) exon of the Grik2 mRNA is encoded by a nucleic acid sequence having at least 85%, 90%, 92%, 95%, 97%, 99%, or 100% sequence identity over at least contiguous nucleotides of SEQ ID NO: 138;
(k) exon 11 of the Grik2 mRNA is encoded by a nucleic acid sequence having at least 85%, 90%, 92%, 95%, 97%, 99%, or 100% sequence identity over at least contiguous nucleotides of SEQ ID NO: 139;
(l) exon 12 of the Grik2 mRNA is encoded by a nucleic acid sequence having at least 85%, 90%, 92%, 95%, 97%, 99%, or 100% sequence identity over at least contiguous nucleotides of SEQ ID NO: 140;
(m) exon 13 of the Grik2 mRNA is encoded by a nucleic acid sequence having at least 85%, 90%, 92%, 95%, 97%, 99%, or 100% sequence identity over at least contiguous nucleotides of SEQ ID NO: 141;
(n) exon 14 of the Grik2 mRNA is encoded by a nucleic acid sequence having at least 85%, 90%, 92%, 95%, 97%, 99%, or 100% sequence identity over at least contiguous nucleotides of SEQ ID NO: 142;
(o) exon of the Grik2 mRNA is encoded by a nucleic acid sequence having at least 85%, 90%, 92%, 95%, 97%, 99%, or 100% sequence identity over at least contiguous nucleotides of SEQ ID NO: 143; and/or
(p) exon 16 of the Grik2 mRNA is encoded by a nucleic acid sequence having at least 85%, 90%, 92%, 95%, 97%, 99%, or 100% sequence identity over at least contiguous nucleotides of SEQ ID NO: 144.

41. The polynucleotide of claim 40, wherein the polynucleotide comprises:
(a) a nucleic acid sequence having at least 85%, 90%, 92%, 95%, 97%, 99%, or 100% sequence identity over at least contiguous nucleotides of SEQ ID NO: 1;
(b) (i) a nucleic acid sequence having at least 85%, 90%, 92%, 95%, 97%, 99%, or 100% sequence identity over at least contiguous nucleotides of SEQ ID NO: 2;
(ii) a nucleic acid sequence having at least 85%, 90%, 92%, 95%, 97%, 99%, or 100% sequence identity over at least contiguous nucleotides of SEQ ID NO: 3;
(iii) a nucleic acid sequence having at least 85%, 90%, 92%, 95%, 97%, 99%, or 100% sequence identity over at least contiguous nucleotides of SEQ ID NO: 30;
(iv) a nucleic acid sequence having at least 85%, 90%, 92%, 95%, 97%, 99%, or 100% sequence identity over at least contiguous nucleotides of SEQ ID NO: 31;
(v) a nucleic acid sequence having at least 85%, 90%, 92%, 95%, 97%, 99%, or 100% sequence identity over at least contiguous nucleotides of SEQ ID NO: 36;
(vi) a nucleic acid sequence having at least 85%, 90%, 92%, 95%, 97%, 99%, or 100% sequence identity over at least contiguous nucleotides of SEQ ID NO: 40;
(vii) a nucleic acid sequence having at least 85%, 90%, 92%, 95%, 97%, 99%, or 100% sequence identity over at least contiguous nucleotides of SEQ ID NO: 59;
(viii) a nucleic acid sequence having at least 85%, 90%, 92%, 95%, 97%, 99%, or 100% sequence identity over at least contiguous nucleotides of SEQ ID NO: 76;
(ix) a nucleic acid sequence having at least 85%, 90%, 92%, 95%, 97%, 99%, or 100% sequence identity over at least contiguous nucleotides of SEQ ID NO: 80;
(x) a nucleic acid sequence having at least 85%, 90%, 92%, 95%, 97%, 99%, or 100% sequence identity over at least contiguous nucleotides of SEQ ID NO: 81;
(xi) a nucleic acid sequence having at least 85%, 90%, 92%, 95%, 97%, 99%, or 100% sequence identity over at least contiguous nucleotides of SEQ ID NO: 92; and/or
(xii) a nucleic acid sequence having at least 85%, 90%, 92%, 95%, 97%, 99%, or 100% sequence identity over at least contiguous nucleotides of SEQ ID NO: 93;
(c) (i) a nucleic acid sequence having at least 85%, 90%, 92%, 95%, 97%, 99%, or 100% sequence identity over at least contiguous nucleotides of SEQ ID NO: 40;
(ii) a nucleic acid sequence having at least 85%, 90%, 92%, 95%, 97%, 99%, or 100% sequence identity over at least contiguous nucleotides of SEQ ID NO: 60;
(iii) a nucleic acid sequence having at least 85%, 90%, 92%, 95%, 97%, 99%, or 100% sequence identity over at least contiguous nucleotides of SEQ ID NO: 68;
(iv) a nucleic acid sequence having at least 85%, 90%, 92%, 95%, 97%, 99%, or 100% sequence identity over at least contiguous nucleotides of SEQ ID NO: 70; and/or
(v) a nucleic acid sequence having at least 85%, 90%, 92%, 95%, 97%, 99%, or 100% sequence identity over at least contiguous nucleotides of SEQ ID NO: 86;
(d) (i) a nucleic acid sequence having at least 85%, 90%, 92%, 95%, 97%, 99%, or 100% sequence identity over at least contiguous nucleotides of SEQ ID NO: 68;
(ii) a nucleic acid sequence having at least 85%, 90%, 92%, 95%, 97%, 99%, or 100% sequence identity over at least contiguous nucleotides of SEQ ID NO: 69; and/or
(iii) a nucleic acid sequence having at least 85%, 90%, 92%, 95%, 97%, 99%, or 100% sequence identity over at least contiguous nucleotides of SEQ ID NO: 70;
(e) (i) a nucleic acid sequence having at least 85%, 90%, 92%, 95%, 97%, 99%, or 100% sequence identity over at least contiguous nucleotides of SEQ ID NO: 4;
(ii) a nucleic acid sequence having at least 85%, 90%, 92%, 95%, 97%, 99%, or 100% sequence identity over at least contiguous nucleotides of SEQ ID NO: 5;
(iii) a nucleic acid sequence having at least 85%, 90%, 92%, 95%, 97%, 99%, or 100% sequence identity over at least contiguous nucleotides of SEQ ID NO: 6;
(vi) a nucleic acid sequence having at least 85%, 90%, 92%, 95%, 97%, 99%, or 100% sequence identity over at least contiguous nucleotides of SEQ ID NO: 56;
(v) a nucleic acid sequence having at least 85%, 90%, 92%, 95%, 97%, 99%, or 100% sequence identity over at least contiguous nucleotides of SEQ ID NO: 57;
(vi) a nucleic acid sequence having at least 85%, 90%, 92%, 95%, 97%, 99%, or 100% sequence identity over at least contiguous nucleotides of SEQ ID NO: 58;
(vii) a nucleic acid sequence having at least 85%, 90%, 92%, 95%, 97%, 99%, or 100% sequence identity over at least contiguous nucleotides of SEQ ID NO: 91;
(viii) a nucleic acid sequence having at least 85%, 90%, 92%, 95%, 97%, 99%, or 100% sequence identity over at least contiguous nucleotides of SEQ ID NO: 94; and/or
(ix) a nucleic acid sequence having at least 85%, 90%, 92%, 95%, 97%, 99%, or 100% sequence identity over at least contiguous nucleotides of SEQ ID NO: 95;
(f) (i) a nucleic acid sequence having at least 85%, 90%, 92%, 95%, 97%, 99%, or 100% sequence identity over at least contiguous nucleotides of SEQ ID NO: 20;
(ii) a nucleic acid sequence having at least 85%, 90%, 92%, 95%, 97%, 99%, or 100% sequence identity over at least contiguous nucleotides of SEQ ID NO: 37;
(iii) a nucleic acid sequence having at least 85%, 90%, 92%, 95%, 97%, 99%, or 100% sequence identity over at least contiguous nucleotides of SEQ ID NO: 38;
(iv) a nucleic acid sequence having at least 85%, 90%, 92%, 95%, 97%, 99%, or 100% sequence identity over at least contiguous nucleotides of SEQ ID NO: 44;
(v) a nucleic acid sequence having at least 85%, 90%, 92%, 95%, 97%, 99%, or 100% sequence identity over at least contiguous nucleotides of SEQ ID NO: 46;
(g) a nucleic acid sequence having at least 85%, 90%, 92%, 95%, 97%, 99%, or 100% sequence identity over at least contiguous nucleotides of SEQ ID NO: 12;
(h) (i) a nucleic acid sequence having at least 85%, 90%, 92%, 95%, 97%, 99%, or 100% sequence identity over at least contiguous nucleotides of SEQ ID NO: 7;
(ii) a nucleic acid sequence having at least 85%, 90%, 92%, 95%, 97%, 99%, or 100% sequence identity over at least contiguous nucleotides of SEQ ID NO: 8;
(iii) a nucleic acid sequence having at least 85%, 90%, 92%, 95%, 97%, 99%, or 100% sequence identity over at least contiguous nucleotides of SEQ ID NO: 96;
(iv) a nucleic acid sequence having at least 85%, 90%, 92%, 95%, 97%, 99%, or 100% sequence identity over at least contiguous nucleotides of SEQ ID NO: 98; and/or
(v) a nucleic acid sequence having at least 85%, 90%, 92%, 95%, 97%, 99%, or 100% sequence identity over at least contiguous nucleotides of SEQ ID NO: 99;
(i) (i) a nucleic acid sequence having at least 85%, 90%, 92%, 95%, 97%, 99%, or 100% sequence identity over at least contiguous nucleotides of SEQ ID NO: 22;
(ii) a nucleic acid sequence having at least 85%, 90%, 92%, 95%, 97%, 99%, or 100% sequence identity over at least contiguous nucleotides of SEQ ID NO: 39;
(iii) a nucleic acid sequence having at least 85%, 90%, 92%, 95%, 97%, 99%, or 100% sequence identity over at least contiguous nucleotides of SEQ ID NO: 62;
(vi) a nucleic acid sequence having at least 85%, 90%, 92%, 95%, 97%, 99%, or 100% sequence identity over at least contiguous nucleotides of SEQ ID NO: 74;
(v) a nucleic acid sequence having at least 85%, 90%, 92%, 95%, 97%, 99%, or 100% sequence identity over at least contiguous nucleotides of SEQ ID NO: 75;
(vi) a nucleic acid sequence having at least 85%, 90%, 92%, 95%, 97%, 99%, or 100% sequence identity over at least contiguous nucleotides of SEQ ID NO: 87;
(vii) a nucleic acid sequence having at least 85%, 90%, 92%, 95%, 97%, 99%, or 100% sequence identity over at least contiguous nucleotides of SEQ ID NO: 88;
(viii) a nucleic acid sequence having at least 85%, 90%, 92%, 95%, 97%, 99%, or 100% sequence identity over at least contiguous nucleotides of SEQ ID NO: 89; and/or
(ix) a nucleic acid sequence having at least 85%, 90%, 92%, 95%, 97%, 99%, or 100% sequence identity over at least contiguous nucleotides of SEQ ID NO: 90;
(j) (i) a nucleic acid sequence having at least 85%, 90%, 92%, 95%, 97%, 99%, or 100% sequence identity over at least contiguous nucleotides of SEQ ID NO: 82;
(ii) a nucleic acid sequence having at least 85%, 90%, 92%, 95%, 97%, 99%, or 100% sequence identity over at least contiguous nucleotides of SEQ ID NO: 83;
(iii) a nucleic acid sequence having at least 85%, 90%, 92%, 95%, 97%, 99%, or 100% sequence identity over at least contiguous nucleotides of SEQ ID NO: 84; and/or
(iv) a nucleic acid sequence having at least 85%, 90%, 92%, 95%, 97%, 99%, or 100% sequence identity over at least contiguous nucleotides of SEQ ID NO: 85;
(k) (i) a nucleic acid sequence having at least 85%, 90%, 92%, 95%, 97%, 99%, or 100% sequence identity over at least contiguous nucleotides of SEQ ID NO: 13;
(ii) a nucleic acid sequence having at least 85%, 90%, 92%, 95%, 97%, 99%, or 100% sequence identity over at least contiguous nucleotides of SEQ ID NO: 14;
(iii) a nucleic acid sequence having at least 85%, 90%, 92%, 95%, 97%, 99%, or 100% sequence identity over at least contiguous nucleotides of SEQ ID NO: 72; and/or
(iv) a nucleic acid sequence having at least 85%, 90%, 92%, 95%, 97%, 99%, or 100% sequence identity over at least contiguous nucleotides of SEQ ID NO: 73;
(I) (i) a nucleic acid sequence having at least 85%, 90%, 92%, 95%, 97%, 99%, or 100% sequence identity over at least contiguous nucleotides of SEQ ID NO: 34;
(ii) a nucleic acid sequence having at least 85%, 90%, 92%, 95%, 97%, 99%, or 100% sequence identity over at least contiguous nucleotides of SEQ ID NO: 35;
(iii) a nucleic acid sequence having at least 85%, 90%, 92%, 95%, 97%, 99%, or 100% sequence identity over at least contiguous nucleotides of SEQ ID NO: 77;
(iv) a nucleic acid sequence having at least 85%, 90%, 92%, 95%, 97%, 99%, or 100% sequence identity over at least contiguous nucleotides of SEQ ID NO: 78; and/or
(v) a nucleic acid sequence having at least 85%, 90%, 92%, 95%, 97%, 99%, or 100% sequence identity over at least contiguous nucleotides of SEQ ID NO: 79;
(m) a nucleic acid sequence having at least 85%, 90%, 92%, 95%, 97%, 99%, or 100% sequence identity over at least contiguous nucleotides of SEQ ID NO: 51;
and/or
(n) (i) a nucleic acid sequence having at least 85%, 90%, 92%, 95%, 97%, 99%, or 100% sequence identity over at least contiguous nucleotides of SEQ ID NO: 9;
(ii) a nucleic acid sequence having at least 85%, 90%, 92%, 95%, 97%, 99%, or 100% sequence identity over at least contiguous nucleotides of SEQ ID NO: 10;
(iii) a nucleic acid sequence having at least 85%, 90%, 92%, 95%, 97%, 99%, or 100% sequence identity over at least contiguous nucleotides of SEQ ID NO: 11;
(iv) a nucleic acid sequence having at least 85%, 90%, 92%, 95%, 97%, 99%, or 100% sequence identity over at least contiguous nucleotides of SEQ ID NO: 15;
(v) a nucleic acid sequence having at least 85%, 90%, 92%, 95%, 97%, 99%, or 100% sequence identity over at least contiguous nucleotides of SEQ ID NO: 16;
(vi) a nucleic acid sequence having at least 85%, 90%, 92%, 95%, 97%, 99%, or 100% sequence identity over at least contiguous nucleotides of SEQ ID NO: 17;
(vii) a nucleic acid sequence having at least 85%, 90%, 92%, 95%, 97%, 99%, or 100% sequence identity over at least contiguous nucleotides of SEQ ID NO: 18;
(viii) a nucleic acid sequence having at least 85%, 90%, 92%, 95%, 97%, 99%, or 100% sequence identity over at least contiguous nucleotides of SEQ ID NO: 27;
(ix) a nucleic acid sequence having at least 85%, 90%, 92%, 95%, 97%, 99%, or 100% sequence identity over at least contiguous nucleotides of SEQ ID NO: 32;
(x) a nucleic acid sequence having at least 85%, 90%, 92%, 95%, 97%, 99%, or 100% sequence identity over at least contiguous nucleotides of SEQ ID NO: 33;
(xi) a nucleic acid sequence having at least 85%, 90%, 92%, 95%, 97%, 99%, or 100% sequence identity over at least contiguous nucleotides of SEQ ID NO: 41;
(xii) a nucleic acid sequence having at least 85%, 90%, 92%, 95%, 97%, 99%, or 100% sequence identity over at least contiguous nucleotides of SEQ ID NO: 49;
(xiii) a nucleic acid sequence having at least 85%, 90%, 92%, 95%, 97%, 99%, or 100% sequence identity over at least contiguous nucleotides of SEQ ID NO: 50;
(xiv) a nucleic acid sequence having at least 85%, 90%, 92%, 95%, 97%, 99%, or 100% sequence identity over at least contiguous nucleotides of SEQ ID NO: 52;
(xv) a nucleic acid sequence having at least 85%, 90%, 92%, 95%, 97%, 99%, or 100% sequence identity over at least contiguous nucleotides of SEQ ID NO: 53;
(xvi) a nucleic acid sequence having at least 85%, 90%, 92%, 95%, 97%, 99%, or 100% sequence identity over at least contiguous nucleotides of SEQ ID NO: 61; and/or
(xvii) a nucleic acid sequence having at least 85%, 90%, 92%, 95%, 97%, 99%, or 100% sequence identity over at least contiguous nucleotides of SEQ ID NO: 63.

42. The polynucleotide of claim 41, wherein sequence identity is determined over at least contiguous nucleotides of any one of SEQ ID NOs: 1-12, 13-18, 20, 22, 27, 30-41, 44, 46, 49-53, 56-63, 68-70, 72-92, or 94-99.

43. The polynucleotide of claim 42, wherein sequence identity is determined over at least contiguous nucleotides of any one of SEQ ID NOs: 1-12, 13-18, 20, 22, 27, 30-41, 44, 46, 49-53, 56-63, 68-70, 72-92, or 94-99.

44. The polynucleotide of claim 43, wherein sequence identity is determined over at least contiguous nucleotides of any one of SEQ ID NOs: 1-12, 13-18, 20, 22, 27, 30-41, 44, 46, 49-53, 56-63, 68-70, 72-92, or 94-99.

45. The polynucleotide of claim 44, wherein sequence identity is determined over the full length of any one of SEQ ID NOs: 1-12, 13-18, 20, 22, 27, 30-41, 44, 46, 49-53, 56-63, 68-70, 72-92, or 94-99.

46. The polynucleotide of claim 1, wherein the polynucleotide comprises a nucleic acid sequence of SEQ ID NO: 68 and SEQ ID NO: 649.

47. The polynucleotide of any one of claims 1-46, wherein the polynucleotide hybridizes to a non-coding sequence of the Grik2 mRNA.

48. The polynucleotide of claim 47, wherein the non-coding sequence comprises a 5′ untranslated region (UTR) of the Grik2 mRNA.

49. The polynucleotide of claim 48, wherein the 5′ UTR is encoded by a polynucleotide having at least 85% sequence identity to the nucleic acid sequence of SEQ ID NO: 126.

50. The polynucleotide of claim 47, wherein the non-coding sequence comprises a 3′ UTR of the Grik2 mRNA.

51. The polynucleotide of claim 50, wherein the 3′ UTR is encoded by a polynucleotide having at least 85% sequence identity to the nucleic acid sequence of SEQ ID NO: 127.

52. The polynucleotide of any one of claims 1-51, wherein the polynucleotide hybridizes to any one of the nucleic acid sequences of SEQ ID NOs: 115-681.

53. The polynucleotide of any one of claims 1-52, wherein the polynucleotide has at least 85% sequence identity to the nucleic acid sequence of any one of SEQ ID NOs: 1-100.

54. The polynucleotide of any one of claims 1-53, wherein the polynucleotide is an antisense oligonucleotide (ASO).

55. The polynucleotide of claim 54, wherein the ASO is a short interfering RNA (siRNA), a short hairpin RNA (shRNA), a microRNA (miRNA), or a short hairpin-adapted miRNA (shmiRNA).

56. The polynucleotide of any one of claims 1-55, wherein the polynucleotide is between 19 to 21 nucleotides.

57. The polynucleotide of claim 56, wherein the polynucleotide is 19 nucleotides.

58. The polynucleotide of claim 57, wherein the polynucleotide is nucleotides.

59. The polynucleotide of claim 58, wherein the polynucleotide is 21 nucleotides.

60. The polynucleotide of any one of claims 1-59, wherein the Grik2 mRNA is encoded by a nucleic acid sequence of SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, or SEQ ID NO: 124.

61. The polynucleotide of any one of claims 1-60, wherein the polynucleotide is capable of reducing a level of Gluk2 protein in a cell.

62. The polynucleotide of claim 61, wherein the polynucleotide reduces a level of GluK2 protein in the cell by at least 10%, at least at least 15%, at least 20%, at least 25%, at least 30%, at least 35%, at least 40%, at least 45%, at least 50%, at least 55%, at least 60%, at least 65%, at least 70%, or at least 75%.

63. The polynucleotide of claim 61 or 62, wherein the cell is a neuron.

64. The polynucleotide of claim 63, wherein the neuron is a hippocampal neuron.

65. The polynucleotide of claim 64, wherein the hippocampal neuron is a dentate granule cell (DGC).

66. A vector comprising the polynucleotide of any one of claims 1-65.

67. The vector of claim 66, wherein the vector is replication-defective.

68. The vector of claim 66 or 67, wherein the vector is a mammalian, bacterial, or viral vector.

69. The vector of any one of claims 66-68, wherein the vector is an expression vector.

70. The vector of claim 68 or claim 69, wherein the viral vector is selected from the group consisting of an adeno-associated virus (AAV), retrovirus, adenovirus, parvovirus, coronavirus, negative strand RNA viruses, orthomyxovirus, rhabdovirus, paramyxovirus, positive strand RNA viruses, picornavirus, alphavirus, a double stranded DNA virus, herpesvirus, Epstein-Barr virus, cytomegalovirus, fowlpox virus, and canarypox virus.

71. The vector of claim 70, wherein the vector is an AAV vector.

72. The vector of claim 71, wherein the AAV vector is an AAV9 or AAVrhvector.

73. The vector of any one of claims 66-72, wherein the vector comprises an expression cassette selected from Table 9.

74. An expression cassette comprising a nucleotide sequence comprising:
(a) a stem-loop sequence comprising, from 5′ to 3′:
(i) a 5′ stem-loop arm comprising a guide nucleotide sequence having at least 85% sequence identity to any one of the guide sequences listed in Table 2 and/or Table 3;
(ii) a loop region, wherein the loop region comprises a microRNA loop sequence;
(iii) a 3′ stem-loop arm comprising a passenger nucleotide sequence complementary or substantially complementary to the guide sequence,
(b) a first flanking region located 5′ to said guide sequence; and
(c) a second flanking region located 3′ to said passenger sequence.

75. The expression cassette of claim 74, wherein the expression cassette comprises any one of the structures described in Table 9.

76. An expression cassette comprising a nucleotide sequence comprising:
(a) a stem-loop sequence comprising, from 5′ to 3′:
(i) a 5′ stem-loop arm comprising a passenger nucleotide sequence which is complementary or substantially complementary to a guide sequence;
(ii) a loop region, wherein the loop region comprises a microRNA loop sequence;
(iii) a 3′ stem-loop arm comprising a guide nucleotide sequence having at least 85% sequence identity to any one of the guide sequences listed in Table 2 and/or Table 3;
(b) a first flanking region located 5′ to said guide sequence; and
(c) a second flanking region located 3′ to said passenger sequence.

77. The expression cassette of claim 76, wherein the expression cassette comprises any one of the structures described in Table 9.

78. The expression cassette of any one of claims 74-77, wherein the first flanking region comprises a polynucleotide having at least 85% sequence identity to the nucleic acid sequence of any one of SEQ ID NOs: 752, 754, 756, 759, 762, 765, or 768.

79. The expression cassette of any one of claims 74-78, wherein the second flanking region comprises a polynucleotide having at least 85% sequence identity to the nucleic acid sequence of any one of SEQ ID NOs: 753, 755, 757, 760, 763, 766, or 769.

80. The expression cassette of any one of claims 74-79, wherein the first flanking region comprises a 5′ spacer sequence and a 5′ flanking sequence

81. The expression cassette of any one of claims 74-80, wherein the second flanking region comprises a 3′ spacer sequence and a 3′ flanking sequence.

82. The expression cassette of any one of claims 74-81, wherein the microRNA loop sequence is a miR-30, miR-155, miR-218-1, or miR-124-3 sequence.

83. The expression cassette of claim 82, wherein the microRNA loop sequence comprises a polynucleotide having at least 85% sequence identity to the nucleic acid sequence of any one of SEQ ID NOs: 758, 761, 764, 767, or 770.

84. The expression cassette of any one of claims 74-83, wherein the expression cassette comprises a promoter selected from the group consisting of a U6 promoter, H1 promoter, 7SK promoter, Apolipoprotein E-Human Alpha 1-Antitrypsin promoter, CAG promoter, CBA promoter, CK8 promoter, mU1a promoter, Elongation Factor 1a promoter, Thyroxine Binding Globulin promoter, Synapsin promoter, RNA Binding Fox-1 Homolog 3 promoter, Calcium/Calmodulin Dependent Protein Kinase II promoter, neuron-specific enolase promoter, Platelet Derived Growth Factor Subunit β, Vesicular Glutamate Transporter promoter, Somatostatin promoter, Neuropeptide Y promoter, Vasoactive Intestinal Peptide promoter, Parvalbumin promoter, Glutamate Decarboxylase 65 promoter, Glutamate Decarboxylase 67 promoter, Dopamine Receptor D1 promoter, Dopamine Receptor D2 promoter, Complement C1q Like 2 promoter, Proopiomelanocortin promoter, Prospero Homeobox 1 promoter, Microtubule Associated Protein 1B promoter, and Tubulin Alpha 1 promoter.

85. The expression cassette of claim 76, wherein the expression cassette comprises a polynucleotide having at least 85% sequence identity to the nucleic acid sequence of any one of SEQ ID NOs: 775, 777, 779, 781, 783-788, 796, 798-801, 803, 805, 807, 809, 813, 817, 819, and 821.

86. An expression cassette comprising, from 5′ to 3′:
(a) a first promoter sequence;
(b) a first guide nucleotide sequence having at least 85% sequence identity to any one of the guide sequences listed in Table 2 and/or Table 3, wherein the first guide nucleotide sequence is operably linked to the first promoter;
(c) a second promoter sequence;
(b) a second guide nucleotide sequence having at least 85% sequence identity to any one of the guide sequences listed in Table 2 and/or Table 3, wherein the second guide nucleotide sequence is operably linked to the second promoter.

87. The expression cassette of claim 86, further comprising a first passenger nucleotide sequence which is complementary or substantially complementary to the first guide nucleotide sequence, wherein the first passenger nucleotide sequence is located 5′ or 3′ relative to the first guide nucleotide sequence.

88. The expression cassette of claim 86 or 87, further comprising a second passenger nucleotide sequence which is complementary or substantially complementary to the second guide nucleotide sequence, wherein the second passenger nucleotide sequence is located 5′ or 3′ relative to the second guide nucleotide sequence.

89. The expression cassette of any one of claims 86-88, further comprising a first 5′ flanking region located 5′ relative to the first guide sequence.

90. The expression cassette of any one of claims 86-89, further comprising a first 3′ flanking region located 3′ relative to the first guide sequence.

91. The expression cassette of any one of claims 86-90, further comprising a second 5′ flanking region located 5′ relative to the second guide sequence.

92. The expression cassette of any one of claims 86-91, further comprising a second 3′ flanking region located 3′ relative to the second guide sequence.

93. The expression cassette of any one of claims 86-92, further comprising a first loop region located between the first guide sequence and the first passenger sequence, wherein the first loop region comprises a first microRNA loop sequence.

94. The expression cassette of any one of claims 86-93, further comprising a second loop region located between the second guide sequence and the second passenger sequence, wherein the second loop region comprises a second microRNA loop sequence.

95. The expression cassette of claim 86, wherein the expression cassette comprises a polynucleotide having at least 85% sequence identity to the nucleic acid sequence of any one of SEQ ID NOs: 785-788.

96. An expression cassette comprising a nucleotide sequence comprising, from 5′ to 3′:
(a) a first promoter sequence;
(b) a first 5′ flanking region located 5′ to a first passenger nucleotide sequence;
(c) a first stem-loop sequence comprising, from 5′ to 3′:
(i) a first 5′ stem-loop arm comprising the first passenger nucleotide sequence which is complementary or substantially complementary to a first guide sequence;
(ii) a first loop region, wherein the first loop region comprises a first microRNA loop sequence;
(iii) a first 3′ stem-loop arm comprising a first guide nucleotide sequence having at least 85% sequence identity to any one of the guide sequences listed in Table 2 and/or Table 3;
(d) a first 3′ flanking region located 3′ to said first guide nucleotide sequence;
(e) a second promoter sequence;
(f) a second 5′ flanking region located 5′ to a second passenger nucleotide sequence;
(g) a second stem-loop sequence comprising, from 5′ to 3′:
(i) a second 5′ stem-loop arm comprising the second passenger nucleotide sequence which is complementary or substantially complementary to a second guide sequence;
(ii) a second loop region, wherein the second loop region comprises a second microRNA loop sequence;
(iii) a second 3′ stem-loop arm comprising a second guide nucleotide sequence having at least 85% sequence identity to any one of the guide sequences listed in Table 2 and/or Table 3;
and
(h) a second 3′ flanking region located 3′ to said second guide nucleotide sequence.

97. An expression cassette comprising a nucleotide sequence comprising, from 5′ to 3′:
(a) a first promoter sequence;
(b) a first 5′ flanking region located 5′ to a first passenger nucleotide sequence;
(c) a first stem-loop sequence comprising, from 5′ to 3′:
(i) a first 5′ stem-loop arm comprising a first passenger nucleotide sequence which is complementary or substantially complementary to a first guide sequence;
(ii) a first loop region, wherein the first loop region comprises a first microRNA loop sequence;
(iii) a first 3′ stem-loop arm comprising a first guide nucleotide sequence having at least 85% sequence identity to any one of the guide sequences listed in Table 2 and/or Table 3;
(d) a first 3′ flanking region located 3′ to said first guide nucleotide sequence;
(e) a second promoter sequence;
(f) a second 5′ flanking region located 5′ to a second guide nucleotide sequence;
(g) a second stem-loop sequence comprising, from 5′ to 3′:
(i) a second 5′ stem-loop arm comprising a guide nucleotide sequence having at least 85% sequence identity to any one of the guide sequences listed in Table 2 and/or Table 3;
(ii) a second loop region, wherein the second loop region comprises a second microRNA loop sequence;
(iii) a second 3′ stem-loop arm comprising a second passenger nucleotide sequence which is complementary or substantially complementary to the second guide sequence;
and
(h) a second 3′ flanking region located 3′ to said second passenger nucleotide sequence.

98. An expression cassette comprising a nucleotide sequence comprising, from 5′ to 3′:
(a) a first promoter sequence;
(b) a first 5′ flanking region located 5′ to a first guide nucleotide sequence;
(c) a first stem-loop sequence comprising, from 5′ to 3′:
(i) a first 5′ stem-loop arm comprising a first guide nucleotide sequence having at least 85% sequence identity to any one of the guide sequences listed in Table 2 and/or Table 3;
(ii) a first loop region, wherein the first loop region comprises a first microRNA loop sequence;
(iii) a first 3′ stem-loop arm comprising a first passenger nucleotide sequence which is complementary or substantially complementary to the first guide sequence;
(d) a first 3′ flanking region located 3′ to said first passenger nucleotide sequence;
(e) a second promoter sequence;
(f) a second 5′ flanking region located 5′ to a second passenger nucleotide sequence;
(g) a second stem-loop sequence comprising, from 5′ to 3′:
(i) a second 5′ stem-loop arm comprising a second passenger nucleotide sequence which is complementary or substantially complementary to a second guide sequence;
(ii) a second loop region, wherein the second loop region comprises a second microRNA loop sequence;
(iii) a second 3′ stem-loop arm comprising a second guide nucleotide sequence having at least 85% sequence identity to any one of the guide sequences listed in Table 2 and/or Table 3;
and
(h) a second 3′ flanking region located 3′ to said second guide nucleotide sequence.

99. An expression cassette comprising a nucleotide sequence comprising, from 5′ to 3′:
(a) a first promoter sequence;
(b) a first 5′ flanking region located 5′ to a first guide nucleotide sequence;
(c) a first stem-loop sequence comprising, from 5′ to 3′:
(i) a first 5′ stem-loop arm comprising a first guide nucleotide sequence having at least 85% sequence identity to any one of the guide sequences listed in Table 2 and/or Table 3;
(ii) a first loop region, wherein the first loop region comprises a first microRNA loop sequence;
(iii) a first 3′ stem-loop arm comprising a first passenger nucleotide sequence which is complementary or substantially complementary to the first guide sequence;
(d) a first 3′ flanking region located 3′ to said first passenger nucleotide sequence;
(e) a second promoter sequence;
(f) a second 5′ flanking region located 5′ to a second guide nucleotide sequence;
(g) a second stem-loop sequence comprising, from 5′ to 3′:
(i) a second 5′ stem-loop arm comprising a guide nucleotide sequence having at least 85% sequence identity to any one of the guide sequences listed in Table 2 and/or Table 3;
(ii) a second loop region, wherein the second loop region comprises a second microRNA loop sequence;
(iii) a second 3′ stem-loop arm comprising a second passenger nucleotide sequence which is complementary or substantially complementary to the second guide sequence;
and
(h) a second 3′ flanking region located 3′ to said second passenger nucleotide sequence.

100. The expression cassette of any one of claims 86-99, wherein the first promoter and/or the second promoter is selected from the group consisting of a U6 promoter, H1 promoter, 7SK promoter, Apolipoprotein E-Human Alpha 1-Antitrypsin promoter, CAG promoter, CBA promoter, CK8 promoter, mU1a promoter, Elongation Factor 1a promoter, Thyroxine Binding Globulin promoter, Synapsin promoter, RNA Binding Fox-1 Homolog 3 promoter, Calcium/Calmodulin Dependent Protein Kinase II promoter, neuron-specific enolase promoter, Platelet Derived Growth Factor Subunit β, Vesicular Glutamate Transporter promoter, Somatostatin promoter, Neuropeptide Y promoter, Vasoactive Intestinal Peptide promoter, Parvalbumin promoter, Glutamate Decarboxylase 65 promoter, Glutamate Decarboxylase 67 promoter, Dopamine Receptor D1 promoter, Dopamine Receptor D2 promoter, Complement C1q Like 2 promoter, Proopiomelanocortin promoter, Prospero Homeobox 1 promoter, Microtubule Associated Protein 1B promoter, and Tubulin Alpha 1 promoter.

101. The expression cassette of any one of claims 86-100, wherein the first 5′ flanking region and/or the second 5′ flanking region comprises a polynucleotide having at least 85% sequence identity to the nucleic acid sequence of any one of SEQ ID NOs: 752, 754, 756, 759, 762, 765, or 768.

102. The expression cassette of any one of claims 86-101, wherein the first 3′ flanking region and/or the second 3′ flanking region comprises a polynucleotide having at least 85% sequence identity to the nucleic acid sequence of any one of SEQ ID NOs: 753, 755, 757, 760, 763, 766, or 769.

103. The expression cassette of any one of claims 86-102, wherein the first microRNA loop sequence and/or the second microRNA loop sequence is a miR-30, miR-155, miR-218-1, or miR-124-3 sequence.

104. The expression cassette of claim 103, wherein the microRNA loop sequence comprises a polynucleotide having at least 85% sequence identity to the nucleic acid sequence of any one of SEQ ID NOs: 758, 761, 764, 767, or 770.

105. The expression cassette of any one of claims 74-104, wherein the expression cassette comprises a 5′-inverted terminal repeat (ITR) sequence on the 5′ end of said expression cassette and a 3′-ITR sequence on the 3′ end of said expression cassette.

106. The expression cassette of claim 105, wherein the 5′-ITR and 3′ ITR sequences are AAV2 5′-ITR and 3′ ITR sequences.

107. The expression cassette of claim 105 or 106, wherein the 5′-ITR sequence comprises a polynucleotide having at least 85% sequence identity to the nucleic acid sequence of SEQ ID NO: 746 or SEQ ID NO: 747.

108. The expression cassette of any one of claims 105-107, wherein the 3′-ITR sequence comprises a polynucleotide having at least 85% sequence identity to the nucleic acid sequence SEQ ID NO: 748, SEQ ID NO: 749, or 789.

109. The expression cassette of any one of claims 74-108, further comprising an enhancer sequence.

110. The expression cassette of claim 109, wherein the enhancer sequence comprises a polynucleotide having at least 85% sequence identity to the nucleic acid sequence of SEQ ID NO: 745.

111. The expression cassette of any one of claims 74-110, further comprising an intron sequence.

112. The expression cassette of claim 111, wherein the intron sequence comprises a polynucleotide having at least 85% sequence identity to the nucleic acid sequence of SEQ ID NO: 743 or SEQ ID NO: 744.

113. The expression cassette of any one of claims 74-112, further comprising one or more polyadenylation signals.

114. The expression cassette of claim 113, wherein the one or more polyadenylation signals is a rabbit beta-globin (RBG) polyadenylation signal or a bovine growth hormone (BGH) polyadenylation signal.

115. The expression cassette of claim 114, wherein the RBG polyadenylation signal comprises a polynucleotide having at least 85% sequence identity to the nucleic acid sequence of SEQ ID NO: 750, SEQ ID NO: 751, or SEQ ID NO: 792.

116. The expression cassette of claim 114, wherein the BGH polyadenylation signal comprises a polynucleotide having at least 85% sequence identity to the nucleic acid sequence of SEQ ID NO: 793.

117. The expression cassette of any one of claims 74-116, wherein the expression cassette is incorporated into the vector of any one of claims 66-73.

118. The expression cassette of clam 96, wherein the expression cassette comprises a polynucleotide having at least 85% sequence identity to the nucleic acid sequence of SEQ ID NO: 785, SEQ ID NO: 787, or SEQ ID NO: 788.

119. The expression cassette of claim 98, wherein the expression cassette comprises a polynucleotide having at least 85% sequence identity to the nucleic acid sequence of SEQ ID NO: 786.

120. The expression cassette of any one of claims 74-119, further comprising a stuffer sequence.

121. The expression cassette of claim 120, wherein the stuffer sequence is positioned at the 3′ end of the expression cassette.

122. The expression cassette of claim 120 or 121, wherein the stuffer sequence has at least 85% sequence identity to the nucleic acid sequence of SEQ ID NO: 815 or SEQ ID NO: 816; optionally, wherein the stuffer sequence has at least 90% sequence identity to the nucleic acid sequence of SEQ ID NO: 815 or SEQ ID NO: 816; optionally, wherein the stuffer sequence has at least 95% sequence identity to the nucleic acid sequence of SEQ ID NO: 815 or SEQ ID NO: 816; ; optionally, wherein the stuffer sequence has at least 99% sequence identity to the nucleic acid sequence of SEQ ID NO: 815 or SEQ ID NO: 816; ; optionally, wherein the stuffer sequence has the nucleic acid sequence of SEQ ID NO: 815 or SEQ ID NO: 816.

123. A method of inhibiting Grik2 expression in a cell comprising contacting the cell with at least one said polynucleotide of any one of claims 1-65, the vector of any one of claims 66-73, or the expression cassette of any one of claims 74-122.

124. The method of claim 123, wherein the polynucleotide specifically hybridizes to a Grik2 mRNA and inhibits or reduces the expression of Grik2 in the cell.

125. The method of claim 123 or 124, wherein the method reduces a level of Gluk2 protein in the cell.

126. The method of claim 125, wherein the method reduces a level of GluK2 protein in the cell by at least 10%, at least 10%, at least at least 15%, at least 20%, at least 25%, at least 30%, at least 35%, at least 40%, at least 45%, at least 50%, at least 55%, at least 60%, at least 65%, at least 70%, or at least 75%.

127. The method of any one of claims 123-126, wherein the cell is a neuron.

128. The method of claim 127, wherein the neuron is a hippocampal neuron.

129. The method of claim 128, wherein the hippocampal neuron is a DGC.

130. The method of claim 129, wherein the DGC comprises an aberrant recurrent mossy fiber axon.

131. A method of treating or ameliorating a disorder in a subject in need thereof comprising administering to the subject at least one said polynucleotide of any one of claims 1-65, the vector of any one of claims 66-73, or the expression cassette of any one of claims 74-122.

132. The method of claim 131, wherein the disorder is an epilepsy.

133. The method of claim 132, wherein the epilepsy is a temporal lobe epilepsy (TLE), chronic epilepsy, and/or a refractory epilepsy.

134. The method of claim 133, wherein the epilepsy is a TLE.

135. The method of claim 134, wherein the TLE is a lateral TLE (ITLE).

136. The method of claim 134, wherein the TLE is a mesial TLE (mTLE).

137. The method of any one of claims 131-136, wherein the subject is a human.

138. A pharmaceutical composition comprising the polynucleotide of any one of claims 1-65, the vector of any one of claims 66-73, or the expression cassette of any one of claims 74-122, and a pharmaceutically acceptable carrier, diluent, or excipient.

139. A kit comprising the pharmaceutical composition of claim 138, and a package insert.

140. The kit of claim 139, wherein the package insert comprises instructions for use of the pharmaceutical composition in the method of any one of claims 131-137.


SEQUENCE LISTING (824 sequences)

SEQ ID NO: 1 - RNA, 21 nt, synthetic construct
  source 1..21: mol_type = other RNA
aagggtagtattgggtagcaa

SEQ ID NO: 2 - RNA, 21 nt, synthetic construct
  source 1..21: mol_type = other RNA
aggcccgaagatggcagccac

SEQ ID NO: 3 - RNA, 21 nt, synthetic construct
  source 1..21: mol_type = other RNA
agcattgcagatggactgcac

SEQ ID NO: 4 - RNA, 21 nt, synthetic construct
  source 1..21: mol_type = other RNA
tgttaacaccactgtatcggt

SEQ ID NO: 5 - RNA, 21 nt, synthetic construct
  source 1..21: mol_type = other RNA
aatcgggtttcggaggtgcct

SEQ ID NO: 6 - RNA, 21 nt, synthetic construct
  source 1..21: mol_type = other RNA
atcgggtttcggaggtgcctg

SEQ ID NO: 7 - RNA, 21 nt, synthetic construct
  source 1..21: mol_type = other RNA
tgtagataactctctgaggag

SEQ ID NO: 8 - RNA, 21 nt, synthetic construct
  source 1..21: mol_type = other RNA
ttagttcacgaaccattccat

SEQ ID NO: 9 - RNA, 21 nt, synthetic construct
  source 1..21: mol_type = other RNA
aataactggggcctgtggctt

SEQ ID NO: 10 - RNA, 21 nt, synthetic construct
  source 1..21: mol_type = other RNA
ttagtgacacttttattggtt

SEQ ID NO: 11 - RNA, 21 nt, synthetic construct
  source 1..21: mol_type = other RNA
ttattatttgggatatggggg

SEQ ID NO: 12 - RNA, 21 nt, synthetic construct
  source 1..21: mol_type = other RNA
gatgttcgctggctttccctt

SEQ ID NO: 13 - RNA, 21 nt, synthetic construct
  source 1..21: mol_type = other RNA
tgttccactgtcagaaaggcg

SEQ ID NO: 14 - RNA, 21 nt, synthetic construct
  source 1..21: mol_type = other RNA
cgttccactgtcagaaaggcg

SEQ ID NO: 15 - RNA, 21 nt, synthetic construct
  source 1..21: mol_type = other RNA
tgatattccatctcctggcaa

SEQ ID NO: 16 - RNA, 21 nt, synthetic construct
  source 1..21: mol_type = other RNA
ataagtgatgaacatctgctt

SEQ ID NO: 17 - RNA, 21 nt, synthetic construct
  source 1..21: mol_type = other RNA
tatttccttgataattggcat

SEQ ID NO: 18 - RNA, 21 nt, synthetic construct
  source 1..21: mol_type = other RNA
tatcctcaattcttctaccat

SEQ ID NO: 19 - RNA, 21 nt, synthetic construct
  source 1..21: mol_type = other RNA
catcctcaattcttctaccat

SEQ ID NO: 20 - RNA, 21 nt, synthetic construct
  source 1..21: mol_type = other RNA
tgtcgattacactgcaaggaa

SEQ ID NO: 21 - RNA, 21 nt, synthetic construct
  source 1..21: mol_type = other RNA
gatgcgtcgagtggtgaccgc

SEQ ID NO: 22 - RNA, 21 nt, synthetic construct
  source 1..21: mol_type = other RNA
ctcgaacataggtaatagcca

SEQ ID NO: 23 - RNA, 21 nt, synthetic construct
  source 1..21: mol_type = other RNA
ggttcgggtagaaatgaggat

SEQ ID NO: 24 - RNA, 21 nt, synthetic construct
  source 1..21: mol_type = other RNA
ttagcgttcggctcctgggtt

SEQ ID NO: 25 - RNA, 21 nt, synthetic construct
  source 1..21: mol_type = other RNA
ctagcgttcggctcctgggtt

SEQ ID NO: 26 - RNA, 21 nt, synthetic construct
  source 1..21: mol_type = other RNA
gttcggctcctgggttcgggt

SEQ ID NO: 27 - RNA, 21 nt, synthetic construct
  source 1..21: mol_type = other RNA
aacgttggtggtgcacacgca

SEQ ID NO: 28 - RNA, 21 nt, synthetic construct
  source 1..21: mol_type = other RNA
tggtgcgcctgaagactggat

SEQ ID NO: 29 - RNA, 21 nt, synthetic construct
  source 1..21: mol_type = other RNA
cggtgcgcctgaagactggat

SEQ ID NO: 30 - RNA, 21 nt, synthetic construct
  source 1..21: mol_type = other RNA
tagcgggtctgtatgtgggga

SEQ ID NO: 31 - RNA, 21 nt, synthetic construct
  source 1..21: mol_type = other RNA
cagcgggtctgtatgtgggga

SEQ ID NO: 32 - RNA, 21 nt, synthetic construct
  source 1..21: mol_type = other RNA
tacgcactaccattcatgctt

SEQ ID NO: 33 - RNA, 21 nt, synthetic construct
  source 1..21: mol_type = other RNA
cacgcactaccattcatgctt

SEQ ID NO: 34 - RNA, 21 nt, synthetic construct
  source 1..21: mol_type = other RNA
ttcgatggttgttgactccat

SEQ ID NO: 35 - RNA, 21 nt, synthetic construct
  source 1..21: mol_type = other RNA
ctcgatggttgttgactccat

SEQ ID NO: 36 - RNA, 21 nt, synthetic construct
  source 1..21: mol_type = other RNA
agcgggtctgtatgtggggaa

SEQ ID NO: 37 - RNA, 21 nt, synthetic construct
  source 1..21: mol_type = other RNA
ttgaacggccacagacaccac

SEQ ID NO: 38 - RNA, 21 nt, synthetic construct
  source 1..21: mol_type = other RNA
aaagcgggtcccgaagcgcca

SEQ ID NO: 39 - RNA, 21 nt, synthetic construct
  source 1..21: mol_type = other RNA
agacgcctgggtttgtaccat

SEQ ID NO: 40 - RNA, 21 nt, synthetic construct
  source 1..21: mol_type = other RNA
aaacgaatgagaccagtgctg

SEQ ID NO: 41 - RNA, 21 nt, synthetic construct
  source 1..21: mol_type = other RNA
ataaacgcagtccacttccaa

SEQ ID NO: 42 - RNA, 21 nt, synthetic construct
  source 1..21: mol_type = other RNA
gaccgcagacacgatcacggc

SEQ ID NO: 43 - RNA, 21 nt, synthetic construct
  source 1..21: mol_type = other RNA
ttcggctcctgggttcgggta

SEQ ID NO: 44 - RNA, 21 nt, synthetic construct
  source 1..21: mol_type = other RNA
taaagcgggtcccgaagcgcc

SEQ ID NO: 45 - RNA, 21 nt, synthetic construct
  source 1..21: mol_type = other RNA
tacggcacccacttccccgat

SEQ ID NO: 46 - RNA, 21 nt, synthetic construct
  source 1..21: mol_type = other RNA
cacggcacccacttccccgat

SEQ ID NO: 47 - RNA, 21 nt, synthetic construct
  source 1..21: mol_type = other RNA
agcgccagggtttatgtcgat

SEQ ID NO: 48 - RNA, 21 nt, synthetic construct
  source 1..21: mol_type = other RNA
gtctccgcttcccaaacccat

SEQ ID NO: 49 - RNA, 21 nt, synthetic construct
  source 1..21: mol_type = other RNA
gacgacagtttgtgcttgggt

SEQ ID NO: 50 - RNA, 21 nt, synthetic construct
  source 1..21: mol_type = other RNA
agcctcgtggaaaccaggggt

SEQ ID NO: 51 - RNA, 21 nt, synthetic construct
  source 1..21: mol_type = other RNA
tgtctcgatatggagaaccca

SEQ ID NO: 52 - RNA, 21 nt, synthetic construct
  source 1..21: mol_type = other RNA
tgacgctggcacttcagggac

SEQ ID NO: 53 - RNA, 21 nt, synthetic construct
  source 1..21: mol_type = other RNA
cgacgctggcacttcagggac

SEQ ID NO: 54 - RNA, 21 nt, synthetic construct
  source 1..21: mol_type = other RNA
agacacgatcacggcatggtc

SEQ ID NO: 55 - RNA, 21 nt, synthetic construct
  source 1..21: mol_type = other RNA
ttccccgatctagcgttcggc

SEQ ID NO: 56 - RNA, 21 nt, synthetic construct
  source 1..21: mol_type = other RNA
tgcaatcgttccatcgaccac

SEQ ID NO: 57 - RNA, 21 nt, synthetic construct
  source 1..21: mol_type = other RNA
ttgaatcgggtttcggaggtg

SEQ ID NO: 58 - RNA, 21 nt, synthetic construct
  source 1..21: mol_type = other RNA
ctgaatcgggtttcggaggtg

SEQ ID NO: 59 - RNA, 21 nt, synthetic construct
  source 1..21: mol_type = other RNA
aaggcccgaagatggcagcca

SEQ ID NO: 60 - RNA, 21 nt, synthetic construct
  source 1..21: mol_type = other RNA
ttgagtcgaagattatacctt

SEQ ID NO: 61 - RNA, 21 nt, synthetic construct
  source 1..21: mol_type = other RNA
ggctagtaacatcatcacctc

SEQ ID NO: 62 - RNA, 21 nt, synthetic construct
  source 1..21: mol_type = other RNA
agatatcaggggagagaggat

SEQ ID NO: 63 - RNA, 21 nt, synthetic construct
  source 1..21: mol_type = other RNA
ggttgcatatttccacaggaa

SEQ ID NO: 64 - RNA, 21 nt, synthetic construct
  source 1..21: mol_type = other RNA
tgcgtcgagtggtgaccgcag

SEQ ID NO: 65 - RNA, 21 nt, synthetic construct
  source 1..21: mol_type = other RNA
ttagtcggagagcatccggga

SEQ ID NO: 66 - RNA, 21 nt, synthetic construct
  source 1..21: mol_type = other RNA
atgcgtcgagtggtgaccgca

SEQ ID NO: 67 - RNA, 21 nt, synthetic construct
  source 1..21: mol_type = other RNA
tatccgggagaaatccagcac

SEQ ID NO: 68 - RNA, 21 nt, synthetic construct
  source 1..21: mol_type = other RNA
cccatagctaatgcctgtttt

SEQ ID NO: 69 - RNA, 21 nt, synthetic construct
  source 1..21: mol_type = other RNA
ttgtcatcattcccatagcta

SEQ ID NO: 70 - RNA, 21 nt, synthetic construct
  source 1..21: mol_type = other RNA
cattcccatagctaatgcctg

SEQ ID NO: 71 - RNA, 21 nt, synthetic construct
  source 1..21: mol_type = other RNA
cccatagctaatgcctgcttt

SEQ ID NO: 72 - RNA, 21 nt, synthetic construct
  source 1..21: mol_type = other RNA
aaaccaccaaatgcctcccac

SEQ ID NO: 73 - RNA, 21 nt, synthetic construct
  source 1..21: mol_type = other RNA
atgataagtgtgaaaaaccac

SEQ ID NO: 74 - RNA, 21 nt, synthetic construct
  source 1..21: mol_type = other RNA
agtcgatgaccttctctcgaa

SEQ ID NO: 75 - RNA, 21 nt, synthetic construct
  source 1..21: mol_type = other RNA
ttcgaacataggtaatagcca

SEQ ID NO: 76 - RNA, 21 nt, synthetic construct
  source 1..21: mol_type = other RNA
gacacctggtgcttccagcgg

SEQ ID NO: 77 - RNA, 21 nt, synthetic construct
  source 1..21: mol_type = other RNA
gtctcgatatggagaacccat

SEQ ID NO: 78 - RNA, 21 nt, synthetic construct
  source 1..21: mol_type = other RNA
tgatatggagaacccatggga

SEQ ID NO: 79 - RNA, 21 nt, synthetic construct
  source 1..21: mol_type = other RNA
gatatggagaacccatgggag

SEQ ID NO: 80 - RNA, 21 nt, synthetic construct
  source 1..21: mol_type = other RNA
cagagcattgcagatggactg

SEQ ID NO: 81 - RNA, 21 nt, synthetic construct
  source 1..21: mol_type = other RNA
cccagagcattgcagatggac

SEQ ID NO: 82 - RNA, 21 nt, synthetic construct
  source 1..21: mol_type = other RNA
taccacgtctgagtcagggtt

SEQ ID NO: 83 - RNA, 21 nt, synthetic construct
  source 1..21: mol_type = other RNA
caccacgtctgagtcagggtt

SEQ ID NO: 84 - RNA, 21 nt, synthetic construct
  source 1..21: mol_type = other RNA
ttgagtcagggttgcaagggt

SEQ ID NO: 85 - RNA, 21 nt, synthetic construct
  source 1..21: mol_type = other RNA
agagctccaactccaaaccag

SEQ ID NO: 86 - RNA, 21 nt, synthetic construct
  source 1..21: mol_type = other RNA
tgatggagctttgatgagctc

SEQ ID NO: 87 - RNA, 21 nt, synthetic construct
  source 1..21: mol_type = other RNA
tataggtaatagccagtggag

SEQ ID NO: 88 - RNA, 21 nt, synthetic construct
  source 1..21: mol_type = other RNA
cataggtaatagccagtggag

SEQ ID NO: 89 - RNA, 21 nt, synthetic construct
  source 1..21: mol_type = other RNA
gagcaactgcaaggtcagctt

SEQ ID NO: 90 - RNA, 21 nt, synthetic construct
  source 1..21: mol_type = other RNA
aggtaatagccagtggagcaa

SEQ ID NO: 91 - RNA, 21 nt, synthetic construct
  source 1..21: mol_type = other RNA
agtcgtcataaatccatccag

SEQ ID NO: 92 - RNA, 21 nt, synthetic construct
  source 1..21: mol_type = other RNA
actgacatagaaggaatcttt

SEQ ID NO: 93 - RNA, 21 nt, synthetic construct
  source 1..21: mol_type = other RNA
tagagactgacatagaaggaa

SEQ ID NO: 94 - RNA, 21 nt, synthetic construct
  source 1..21: mol_type = other RNA
tgtcataaatccatccagcaa

SEQ ID NO: 95 - RNA, 21 nt, synthetic construct
  source 1..21: mol_type = other RNA
tatcagtcgtcataaatccat

SEQ ID NO: 96 - RNA, 21 nt, synthetic construct
  source 1..21: mol_type = other RNA
ttcatatgtaaagccaaggat

SEQ ID NO: 97 - RNA, 21 nt, synthetic construct
  source 1..21: mol_type = other RNA
actgacatagaaggaatcctt

SEQ ID NO: 98 - RNA, 21 nt, synthetic construct
  source 1..21: mol_type = other RNA
aatggacaatggaatggaatg

SEQ ID NO: 99 - RNA, 21 nt, synthetic construct
  source 1..21: mol_type = other RNA
atggaatggaatggttcgtga

SEQ ID NO: 100 - RNA, 21 nt, synthetic construct
  source 1..21: mol_type = other RNA
tagtcggagagcatccgggag

SEQ ID NO: 101 - RNA, 21 nt, synthetic construct
  source 1..21: mol_type = other RNA
taatgttagtcatgtccaccg

SEQ ID NO: 102 - protein, 908 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRSFCSAMVEELRMSLKCQRRLKHKPQAPVIVKTEEVINMHTFNDRRLPGKETMA

SEQ ID NO: 103 - protein, 869 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKESSIWLVPPYHPDTV

SEQ ID NO: 104 - protein, 584 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARF

SEQ ID NO: 105 - protein, 832 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRSFCSAMVEELRMSLKCQRRLKHKPQAPVIVKTEEVINMHTFNDRRLPGKETMA

SEQ ID NO: 106 - protein, 892 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRAKTKLPQDYVFLPILESVSISTVLSSSPSSSSLSSCS

SEQ ID NO: 107 - protein, 682 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKESSIWLVPPYHPDTV

SEQ ID NO: 108 - protein, 687 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRAKTKLPQDYVFLPILESVSISTVLSSSPSSSSLSSCS

SEQ ID NO: 109 - protein, 908 aa, Mus musculus [sequence omitted]

SEQ ID NO: 110 - protein, 869 aa, Mus musculus
MKIISPVLSNLVFSRSIKVLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPSSGLNMTESQKGKPANITDSLSNRSLIVTTILEEPYVLFKKSDKPLYGNDRFEGYCIDLLRELSTILGFTYEIRLVEDGKYGAQDDVNGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKESSIWLVPPYHPDTV

SEQ ID NO: 111 - protein, 908 aa, Mus musculus [sequence omitted]

SEQ ID NO: 112 - protein, 908 aa, Macaca mulatta; identical to SEQ ID NO: 102

SEQ ID NO: 113 - protein, 908 aa, Macaca mulatta; identical to SEQ ID NO: 102

SEQ ID NO: 114 - protein, 908 aa, Rattus norvegicus
MKIISPVLSNLVFSRSIKVLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDVNGQWNGMVRELIDHKADLAVAPLAITYVREKVIDFSKPFMTLGISILYRKPNGTNPGVFSFLNPLSPDIWMYVLLACLGVSCVLFVIARFSPYEWYNPHPCNPDSDVVENNFTLLNSFWFGVGALMRQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRSFCSAMVEELRMSLKCQRRLKHKPQAPVIVKTEEVINMHTFNDRRLPGKETMA

SEQ ID NO: 115 - DNA, 4592 bp, Homo sapiens
  source 1..4592: mol_type = other DNA
gctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctatgaccatgccgtgatcgtgtctgcggtcaccactcgacgcatcctcatttctacccgaacccaggagccgaacgctagatcggggaagtgggtgccgtgcgtgtgggcacagaaacaccatgaagattattttcccgattctaagtaatccagtcttcaggcgcaccgttaaactcctgctctgtttactgtggattggatattctcaaggaaccacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagccagaatgtttcctgtggaaatatgcaacctgtgcaaaataaaatgagttacctcatgccgctgtgtctatgaactagagactctgtgatctaagcagttgcaatgatcagacttgatttacaagcatcatggatcaaccaagttacacggggttacactgttaatcatgggttcctcccttcttctgagtgaatgttaacatgcgcattttgtggctgatttcaaatgcagtccagtgagaaattacaggttccttttgaagctcaactgttgccaggagatggaatatcaatgcccaacagggcaaccaataaaagtgtcactaagaatataaatatttggaatcagcaaaaactgtagtgttacaggaaacagtacagtcttctgaacacccagatcatagaggtgatgatgttactagcccccaactactcagtataattattgtctgaatgcaaagtatgtgtttataggatgtgaaaaaatgtaatgcaaaacaaatttgaatcccatggcagttggaatataaagcagatgttcatcacttattttccttttttcttttcttatttttttttttgacagtctgtgtcactgattgagatagaaatgccaattatcaaggaaataatgttttcttaagttccctaaggcagaagatttaacatgcaattctaccagatcccttcctattcccccaacaccttttctctaacccccatatcccaaataataataataataataataataataataataataataataaaagcagttggttcagtgattctgaattaaaaggataatgttttgcaatgttcaagttgtaaaaactggccgagtattggctgtgtggaagactaaagctttcattctaacattcagacatagcaatccaaacccttgttcctgctgtaaatgaacttgatggagcatgggcagatttcagtgatacgagaaaggggactggtcatctatagaaaaatctgtgagagaacttggaagtggactgcgtttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagaaaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataacagttgagtgcacacagtttgctgtttgaatccaatgcacaaaattaaaaaaaatcattaaaactatgttcattttactttca

SEQ ID NO: 116 - DNA, 2727 bp, Homo sapiens
  source 1..2727: mol_type = other DNA
atgaagattattttcccgattctaagtaatccagtcttcaggcgcaccgttaaactcctgctctgtttactgtggattggatattctcaaggaaccacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataa

SEQ ID NO: 117 - DNA, 2610 bp, Homo sapiens
  source 1..2610: mol_type = other DNA
atgaagattattttcccgattctaagtaatccagtcttcaggcgcaccgttaaactcctgctctgtttactgtggattggatattctcaaggaaccacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaaggaatcttctatttggttagtgccaccataccatccagacactgtttag

SEQ ID NO: 118 - DNA, 2679 bp, Homo sapiens
  source 1..2679: mol_type = other DNA
atgaagattattttcccgattctaagtaatccagtcttcaggcgcaccgttaaactcctgctctgtttactgtggattggatattctcaaggaaccacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagagagccaagactaagttacctcaagactatgtattcctccctattttggagtcagtttccatttctacagtgttgtcatcatcaccatcttcatcatcattatcatcatgttcttaa

SEQ ID NO: 119 - DNA, 10568 bp, Homo sapiens
  source 1..10568: mol_type = other DNA
acttgcgctctcgctggcggctgcgtggccgaggctgggagcccgggacttcccgctgaaccgcctcctgccgcagctctgagaggactaccccagtccctaccctccctcttcaccctagccgcaggctcgcgcggctggacattgtgcttgctggatttttcccggatgctcccagactaacatggatgtcccaccatcccttgcagtggaagcttgctccttggcgcagtgagtgaagaacatgcagagactgctaatgggtttgggaagcggagactccttcctctttctgtgaccatgccgtgattgtgtttgcggccactattccacgcatccttcttctcgtccaagcccggagcctaacgctagatcggggaagtgggtgccgcgcgcgcaggcacggaaacatcatgaagattatttccccagttttaagtaatctagtcttcagtcgctccattaaagtcctgctctgcttgttgtggatcggatattcgcaaggaaccacacatgtgttaagattcggtggtatatttgaatatgtggaatctggccctatgggagctgaagaacttgcattcagatttgctgtgaatacaatcaacaggaacaggactctgctacccaataccacgttaacatatgatacacagaagatcaatctctatgacagttttgaagcatctaagaaagcttgtgatcagctgtctcttggggtggctgccatcttcggtccttcacacagttcatcagcaaatgctgttcagtccatctgcaatgctctgggggttcctcacatacagacccgctggaagcaccaggtgtcagacaataaggattccttctatgtcagtctctacccagacttctcttccctcagccgtgccatcttggatttggtgcagttttttaagtggaaaactgtcacagttgtgtatgacgacagcactggtctcattcgcttgcaagagctcatcaaagctccatcaaggtacaatcttcgacttaaaattcgtcagctgccagctgatacaaaagatgcaaagcctttgctgaaagagatgaagaggggcaaggagttccacgtgatcttcgactgcagccatgaaatggcagcaggcattttaaagcaggcattagctatgggaatgatgacagaatactaccactatatatttacgactctggacctcttcgctcttgatgtggagccctacagatacagtggcgtaaatatgacagggttcagaatactaaatacagagaatacccaagtctcctccatcatcgagaagtggtcgatggaacggttacaggcacctccaaaacctgactcaggtttgctggatggatttatgacgactgatgctgctctgatgtatgatgcagtgcacgttgtgtctgtagctgtccaacagtttccccagatgacagtcagctccttgcaatgcaatcgacacaaaccctggcgctttgggactcgcttcatgagcctaattaaagaggctcattgggaaggtcttacaggcagaattacatttaacaaaaccaatggattgcgaacagattttgatttggatgtgatcagtctcaaggaagaaggtctggagaagattgggacttgggatccatccagtggcctgaatatgacagaaagtcagaaagggaagccagcaaatattacagattcattgtctaatcgttctttgattgttaccaccattttggaagaaccatatgtcctgtttaagaagtctgacaaacctctctatgggaatgatcgatttgaaggctactgtattgatcttctacgagagttatctacaatccttggctttacatatgaaattaggcttgtggaggatgggaaatatggagcccaggatgatgtgaatggacaatggaatggaatggttcgtgagctaattgatcataaagctgaccttgcagttgctccactggctattacctatgttcgtgagaaggtcatcgacttttcaaagccgtttatgactcttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccctatgagtggtataatccacacccttgcaaccctgactcagacgtggtggaaaacaattttaccttgctaaatagtttctggtttggagttggagctctcatgcagcaaggttctgagctcatgcccaaagcactctccaccaggatagtgggaggcatttggtggtttttcacacttatcatcatttcttcgtataccgctaacctagccgcctttctgaccgtggaacgcatggagtcgcctattgactctgctgacgatttagctaagcaaaccaagatagaatatggagcagtagaggacggcgcaaccatgacgtttttcaagaaatcaaaaatctcaacgtatgataaaatgtgggcatttatgagcagcaggagacagtctgtgcttgtcaaaagcaatgaggaagggattcaacgtgtcctcacctccgattatgctttcttaatggagtcaacgaccatcgagtttgttacccagcggaactgtaacctcacgcagattggtggccttatagactccaaaggctatggtgttggcactcccatgggttctccatatcgagacaaaatcaccatagccattcttcagctgcaggaggaaggcaagctgcacatgatgaaggagaagtggtggcgaggcaatggctgcccagaggaggagagcaaagaggccagtgctctaggggtgcagaatattggtggtatcttcattgtcctggcagccggcttggtgctctcagtttttgtggcagtgggagagtttttatacaaatccaaaaaaaacgctcaattggaaaagaggtccttctgtagcgccatggtggaagaactgagaatgtctctgaagtgccagcgtcggctcaaacataagccacaggccccagttattgtgaaaacagaagaagttatcaacatgcacacatttaacgacagaaggttgccaggtaaagaaaccatggcatgaagctgggaggccaatcacccaagcacaaactgtcgtcttttttttttttttcaaacaatttagcgagaatgtttcctgtggaaatatgcaacctgtgcaaaataaaatgagttacctcatgccgctgtgtctatgaactagagactcttgtgatctaagcagtttcagtgatcagacttgatttacaagcaccatggatcgacaaagttacacggggttacactgtttatcatgggttcctcccttcctttgagtgaatgttacatgaaaatgttgtggctggtttcaaatgcagtccagagagaaactgctggttccttctgaagctcaactgttgtcaggagatggaatgttggggcccaaaaggataaccaataaaaatgccataatttataaaagcaaaacaaaaagcgtgtgaaatctgcaaaaattgtagtgtcacaagaaacagtatagtcccatggtcaccaacaaaatgaggtgataatgttactagcccccaatactcagtaaaatcatcatctgaatagataatgtgttcatagaatgtggaaaaaatgtaatgcaaaacatatcagtattcaatcaaagtggaacagaaagcagaccaccatcagttattttcctttctcaatagtctgtgtcatggattgtgatatagatggcaattatctatctaattgttttcttaaaatacccatggcaaatattttaaaatgcaacttgctcccaggaacccctaccctaacctacactagaaataaaaaagccaccactggtataaagattctgatgtaaaagatatgtttttcaatccttgtcatgaattgtaaaacagggctcagtattactggttatatggaagactgaagctttcactctgacattctgatatgtcagctgaaactctccttcctcctggaaaggaccttgatggagcctgggcagattccattgataagactggggacttgtcacctatacagaactacgtgacagaactttgaggtggactgcatttaacaatagtcacaatgttaaaagaacaaaattcttgagcagttttttttttctgttttgttttaaaaaatgttcaggtttatttgtggaaatgcaagatttctatgaaaatagtttttgtatggaaatttttgtaatactttttatcaacaaaataagaacatgtgttcctgtcaggggtgtgatgtcaagcatgaacggtagtgcgtgtgcaccaccaacgtttggtgaaaactatttttatcaagaaaaaggaatcatagaagagaaatattttcaagttagatactataaaagctaggtgcactaccaccacggcttgtcacgccacacccctgagtcccacaaggcagataacatattgtaatgaacagttgtgtgtaaaatgataaaagacacagacctcttgacaacattgtgaaaacagttgagtgcacacagtttgctgtttgaatccaatgcacaaaaattttacaaaactccattaaaattatgtccattttactttcagctttggctttgatttttctcttgcatgtgtaaatgaatgtaacatggtggttttgtatagaaaatatacatcaaggggtcttaggatctcaaagttagaatcttcccaacttacagcaaaaaggaaaaggccatcctggaggtgctcctctctttctctctcccttcctctgtctctctctttgactctgtctctttgtgtctcagtctttctctatatcagtttttctctgtccctctctacctctggctctatcactctctgtctcatttacacacatacacacacacacacacacacacacacacacacgaataaagacatatacattggttttagaattagggtagctggaataaaaagaatatgattgtagagatggcaacctttatcttatctcatttgtagctggaaattgactaagttcactgtgctgcattatgttgtggaatggtaattatctactttggttcaactcatatccaatttcagaattttctgtgcattgatacttcaataatcatcagcagaggaacaaaaagggaaaagtttagaattaataattaattttagatcctaacatattaatagaaacaaactataacagttttacgttttgaaaatcaaatctgtaagattcaacttattttcctgattaattaattaattaattctaagtgtgcaattataattggaatctgacaaaaaaaaaaccccactggaaaagtttccataatgtatttcttaaaatagtaaaaattgcataatcaaattatctcaaattaattaagatttatatatgtgagcactttaaatattttatgctatgattattatcagatttcaatgattattttgttcaagctacaaatgtagctatacaaatcactgctaaagtagcagtactggtgtattagtgccaaccaagtttaaataaggaaaataatataagtattcagattattaagatgctgtttttaacaaacaaattttaaattttatgaaaatatgaatttgtaaaggaaacaaacttcattattaatattatggggaaattctgtgaatatatacaatctgacacatgtagaatttgcacattcgatgagaaactgtgtcaaaaatgatcaattgaagcaactcatttaataaaaaagaactctcactaagcaatgctttaatatgttttaaagatataattttaaacacttggaaatcttatatatgtgagtataaaaacacattaaatattcatatcttaatttacttaaaagagttttaacttcaatttattactgaatttaataatcataaacattgggtataaaattacaactttattgttaagcagccagaagagaatgaattctggtaaaatttgataatgattttattgaaaaattgaattaagttcttaaatatgtaacaaccttactagacattaactttataaatgatattaaatgtttataatatattcttttacattctaattctaattaatacttcatacatgtataagtacttaaattttccaaaacacctggtcataatatatatattaattttaattataataaaaatgctaattactttcatacataatatcaaacaatgaaaaaactctaggttggaagcatgtaagagttctcagcattttctagaggaaaaatcaaaagcaaagagaagctaatacctgttctaggcaacatcagaacctatgaattgagaggatgaatggaggctcatggacttcagctatagaaaccaacaaggatggagacaccagaggttttatacccacatttactaccactaaataatcagtatctccctgggaacaccaaagaaacacacataattaccactgaacaaccctaactctcatgggaacacaagagaaatatagtctagtttataaatgtgtaaaatgaagaatagtttattgatgggaatatttcagatattttgttgctcacttatatctacatataagtgcatatgtaataaaaattcatatcccatggcaccattcttgggtggtatagatatgaaacattgctggtttcaaaatgtttaaggattgtgtcaacttttgatgtctgtttactttgaaatatattgtaactgcagaaaagtgcatatacaaggcatatatacacagaattgctgctagttgttatagttttaaacacagttattgtgccatttaacccaggctcaatgaaagttctctgacttgctgataacattttcagggaagaaattacagtgatctttgatcatgaaatgtataattaactcatctgtgtttgactatgccaatagagtgtgtacagcattgtagagaaacagtcttgggattttgtggcagcttttaaaggtagaatcatagcaatgaaaactttggttatgcctgtcctacctaaatgtatacaaaatttgaaccattttcctccattgtgagctgatacgtgaccagatctgcagacatgtaaaattgtgaagttattcccttgtctttaggtatttgcctcaatgcaaattcaaatgtaataattttttttatttctcttaaaatatgtcagttaattatttgattttttctcaattttggtggttttatagaaatgctgaataggctcaatgggttttagatgttttatgtctattattctactctagtctataattatctggatattcattgactcctagctcaaaattagttccttggaaaatttgaagactttccactatttcctcttccccctcatctatttcccattttctcccccatttccatctctttcccccattcctcatcctttatatatatatatatatatatatatacatatatatatatatatatgtgtgtgtgtgtgtgtgtgtgtgtgtgtgtgtgtgtgtgtgtgtacttgcttttttattatcttgtctaatgttgagattgctgtggaaaacctaaaatcatgatcagatggtcacagttctttgatcatcttcttcttcttcttcttcttcttcttcttcttcttcttcttcttcttcttcttcttcttcttcttcttcttcttcttcctcttcctcctcctcctcctcttcctcctcctcttcctcttcgtcgtcttctccttctcctccttcttttccttctcttcctccacctcttcctttcttcttcttttccctcttcttcctccttctcttgtgcctcctctttttgtttttcttacttctctctttgtctctatctctatgtttctgtttctttgtctctgtctctctctctgtgtctctgtctgtcagtctctgtcggcctgtctgtctttctctctgtctcagatgtagattgatctgtatcaattgtactaacccttactagactccttaataaagctactatttttcaattttaagaaaagaccttgaaataatacttcattgtcttcctttcttcatatttcctttgttctctctaaatctgttcttatgacttgtaattacttaactttgaatcttcagtctatttacttttctatttctctatattgttattttaagtctataatctaaaatacttttttagtttctcctatctttgacattgttttaatttctgtaagttactgtttaattcctatgacatcttcacttatatcatagtagttgatcataattgttctttattcccatcattaggaaaagacattgtatattcctgttcttttctatttttttcacctgagttgaaggtattattgagataacagttgagtcatttaccctccagttactcctcccagttcttttcactttctctcccacatgtatctacttcctgtttgtctctcattcaaaaaggaaaggcctctaagagataacagcaaaatttaacaacataaaatataataacataaaacacacaagcaaaaactgtcacatcaaagttgaacaagataaaccaacaaaacaaaaagagcctcaagaaggcagaagaatcagagagcctcttgttcacttacctcagttgaattagaattggttcttatcctgtgggtagtgacccatttagaggtagaatgaccctttcttaaagttttcctaagacaatcagacaatatagatgtttacattatgatttataaatagcaaaattatagttatgaaatagaggcaaaaataattttatggttttgggtcattacaacattaagaaccatatttgagggttctagccttaggaaggttgaaaaccactgaattataaagggccttgtctccttgtgtcctctacccttttgaatctttcacatttctcacctcatcttctgtgggtgggatttagtggagacatcccactgaaagctgtgttccaaatgcatctgtttgttaaatgtcatccgtcaactcttactgttctctttatgatgatgtaaagtagaataaacatcactggcaaatttttgtttatgttaagttgtgacagaatgttcatctaacttgagtaatcctgtaggtgaatatatgtggactacttgatgaataatctgtccaagtacctccgtttggtttttacttcttgggtatattagatttgcagagaggtgtcttttaacttcattcctaatgccttgaaatcctgatcactatatttcagccaccagagtcaagaacttatcactgcaaattctgccctgtgtgtgagtgaataaagcaatttttgtcccatttattctgaaaatttgcgtttaattatatctcacattcttcatactagaatatacattttcctcttttaaaaatatgcacacctgacagactgtgtttgaaatagtgggaacaccatactctatggttccagtgtctgttttgaccatcacattactatcttctctcccaagggccaagtctaccaagtggaatatgcctttaatgctattaaccaggctggtcttatacttgtagctgtcagaggaaaagactgctcaggttttgtcacatagatgaaagtacatgacaaattactagattccaacacacttgttgaagataactgaaaccagtggttgtataatgatgggagtaacagctgacagactccaggtacaaatggcacactatgaggtagttaattggaaagacaaaggtaactatgagtttcctgtggacatgctatgtaaaagaattgctgatattactcaagtcaacacacagaataatgaaatgaggctgcttcattgttgtttgatttttttttactggtataaatgaaaaaacaaggccctcaagtgtactagtggaatcctataggtgttaagccatgacagcagaagttaaatgaatataaccaaccagcttccttgaaaacaataagtgaggaagaaaattgattggatatttgaataaacatttagataatgtaattacatgtctgcctactattctgtcgattggtttcaaactttcagaaatagaaattggaagagttagagttgaaaatactcgattcaggattcttacagaagcagagattggcactcatcttgttgttctagcagagagactgaacattgtcatcagtttaccaaatctgtgatgccacttgcctgtgtgtttgataaaaaccaacatcatagaggctccacagcttaaaatggaacctcttccactcctgccactgagctgcttaggactctgtataaataaaaacagtccttttggaaaaataaatatgtacactgtacttaaaaataaacacatgaaatttttatgtgctacgttaaaacctaactccaaaatttaaagaaacgctacaattgtccacatccattaaaagactccttgttattttatgttctcttttgtaccactattaaattgattctctattgcta

SEQ ID NO: 120 - DNA, 10655 bp, Mus musculus
  source 1..10655: mol_type = other DNA
acttgcgctctcgctggcggctgcgtggccgaggctgggagcccgggacttcccgctgaaccgcctcctgccgcagctctgagaggactaccccagtccctaccctccctcttcaccctagccgcaggctcgcgcggctggacattgtgcttgctggatttttcccggatgctcccagactaacatggatgtcccaccatcccttgcagtggaagcttgctccttggcgcagtgagtgaagaacatgcagagactgctaatgggtttgggaagcggagactccttcctctttctgtgaccatgccgtgattgtgtttgcggccactattccacgcatccttcttctcgtccaagcccggagcctaacgctagatcggggaagtgggtgccgcgcgcgcaggcacggaaacatcatgaagattatttccccagttttaagtaatctagtcttcagtcgctccattaaagtcctgctctgcttgttgtggatcggatattcgcaaggaaccacacatgtgttaagattcggtggtatatttgaatatgtggaatctggccctatgggagctgaagaacttgcattcagatttgctgtgaatacaatcaacaggaacaggactctgctacccaataccacgttaacatatgatacacagaagatcaatctctatgacagttttgaagcatctaagaaagcttgtgatcagctgtctcttggggtggctgccatcttcggtccttcacacagttcatcagcaaatgctgttcagtccatctgcaatgctctgggggttcctcacatacagacccgctggaagcaccaggtgtcagacaataaggattccttctatgtcagtctctacccagacttctcttccctcagccgtgccatcttggatttggtgcagttttttaagtggaaaactgtcacagttgtgtatgacgacagcactggtctcattcgcttgcaagagctcatcaaagctccatcaaggtacaatcttcgacttaaaattcgtcagctgccagctgatacaaaagatgcaaagcctttgctgaaagagatgaagaggggcaaggagttccacgtgatcttcgactgcagccatgaaatggcagcaggcattttaaagcaggcattagctatgggaatgatgacagaatactaccactatatatttacgactctggacctcttcgctcttgatgtggagccctacagatacagtggcgtaaatatgacagggttcagaatactaaatacagagaatacccaagtctcctccatcatcgagaagtggtcgatggaacggttacaggcacctccaaaacctgactcaggtttgctggatggatttatgacgactgatgctgctctgatgtatgatgcagtgcacgttgtgtctgtagctgtccaacagtttccccagatgacagtcagctccttgcaatgcaatcgacacaaaccctggcgctttgggactcgcttcatgagcctaattaaagaggctcattgggaaggtcttacaggcagaattacatttaacaaaaccaatggattgcgaacagattttgatttggatgtgatcagtctcaaggaagaaggtctggagaagattgggacttgggatccatccagtggcctgaatatgacagaaagtcagaaagggaagccagcaaatattacagattcattgtctaatcgttctttgattgttaccaccattttggaagaaccatatgtcctgtttaagaagtctgacaaacctctctatgggaatgatcgatttgaaggctactgtattgatcttctacgagagttatctacaatccttggctttacatatgaaattaggcttgtggaggatgggaaatatggagcccaggatgatgtgaatggacaatggaatggaatggttcgtgagctaattgatcataaagctgaccttgcagttgctccactggctattacctatgttcgtgagaaggtcatcgacttttcaaagccgtttatgactcttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccctatgagtggtataatccacacccttgcaaccctgactcagacgtggtggaaaacaattttaccttgctaaatagtttctggtttggagttggagctctcatgcagcaaggttctgagctcatgcccaaagcactctccaccaggatagtgggaggcatttggtggtttttcacacttatcatcatttcttcgtataccgctaacctagccgcctttctgaccgtggaacgcatggagtcgcctattgactctgctgacgatttagctaagcaaaccaagatagaatatggagcagtagaggacggcgcaaccatgacgtttttcaagaaatcaaaaatctcaacgtatgataaaatgtgggcatttatgagcagcaggagacagtctgtgcttgtcaaaagcaatgaggaagggattcaacgtgtcctcacctccgattatgctttcttaatggagtcaacgaccatcgagtttgttacccagcggaactgtaacctcacgcagattggtggccttatagactccaaaggctatggtgttggcactcccatgggttctccatatcgagacaaaatcaccatagccattcttcagctgcaggaggaaggcaagctgcacatgatgaaggagaagtggtggcgaggcaatggctgcccagaggaggagagcaaagaggccagtgctctaggggtgcagaatattggtggtatcttcattgtcctggcagccggcttggtgctctcagtttttgtggcagtgggagagtttttatacaaatccaaaaaaaacgctcaattggaaaaggaatcttctatttggttagtgccaccataccatccagacactgtttagtaaacttttgaaactttctaaaagaggtttttaatgatgaggtccttctgtagcgccatggtggaagaactgagaatgtctctgaagtgccagcgtcggctcaaacataagccacaggccccagttattgtgaaaacagaagaagttatcaacatgcacacatttaacgacagaaggttgccaggtaaagaaaccatggcatgaagctgggaggccaatcacccaagcacaaactgtcgtcttttttttttttttcaaacaatttagcgagaatgtttcctgtggaaatatgcaacctgtgcaaaataaaatgagttacctcatgccgctgtgtctatgaactagagactcttgtgatctaagcagtttcagtgatcagacttgatttacaagcaccatggatcgacaaagttacacggggttacactgtttatcatgggttcctcccttcctttgagtgaatgttacatgaaaatgttgtggctggtttcaaatgcagtccagagagaaactgctggttccttctgaagctcaactgttgtcaggagatggaatgttggggcccaaaaggataaccaataaaaatgccataatttataaaagcaaaacaaaaagcgtgtgaaatctgcaaaaattgtagtgtcacaagaaacagtatagtcccatggtcaccaacaaaatgaggtgataatgttactagcccccaatactcagtaaaatcatcatctgaatagataatgtgttcatagaatgtggaaaaaatgtaatgcaaaacatatcagtattcaatcaaagtggaacagaaagcagaccaccatcagttattttcctttctcaatagtctgtgtcatggattgtgatatagatggcaattatctatctaattgttttcttaaaatacccatggcaaatattttaaaatgcaacttgctcccaggaacccctaccctaacctacactagaaataaaaaagccaccactggtataaagattctgatgtaaaagatatgtttttcaatccttgtcatgaattgtaaaacagggctcagtattactggttatatggaagactgaagctttcactctgacattctgatatgtcagctgaaactctccttcctcctggaaaggaccttgatggagcctgggcagattccattgataagactggggacttgtcacctatacagaactacgtgacagaactttgaggtggactgcatttaacaatagtcacaatgttaaaagaacaaaattcttgagcagttttttttttctgttttgttttaaaaaatgttcaggtttatttgtggaaatgcaagatttctatgaaaatagtttttgtatggaaatttttgtaatactttttatcaacaaaataagaacatgtgttcctgtcaggggtgtgatgtcaagcatgaacggtagtgcgtgtgcaccaccaacgtttggtgaaaactatttttatcaagaaaaaggaatcatagaagagaaatattttcaagttagatactataaaagctaggtgcactaccaccacggcttgtcacgccacacccctgagtcccacaaggcagataacatattgtaatgaacagttgtgtgtaaaatgataaaagacacagacctcttgacaacattgtgaaaacagttgagtgcacacagtttgctgtttgaatccaatgcacaaaaattttacaaaactccattaaaattatgtccattttactttcagctttggctttgatttttctcttgcatgtgtaaatgaatgtaacatggtggttttgtatagaaaatatacatcaaggggtcttaggatctcaaagttagaatcttcccaacttacagcaaaaaggaaaaggccatcctggaggtgctcctctctttctctctcccttcctctgtctctctctttgactctgtctctttgtgtctcagtctttctctatatcagtttttctctgtccctctctacctctggctctatcactctctgtctcatttacacacatacacacacacacacacacacacacacacacacgaataaagacatatacattggttttagaattagggtagctggaataaaaagaatatgattgtagagatggcaacctttatcttatctcatttgtagctggaaattgactaagttcactgtgctgcattatgttgtggaatggtaattatctactttggttcaactcatatccaatttcagaattttctgtgcattgatacttcaataatcatcagcagaggaacaaaaagggaaaagtttagaattaataattaattttagatcctaacatattaatagaaacaaactataacagttttacgttttgaaaatcaaatctgtaagattcaacttattttcctgattaattaattaattaattctaagtgtgcaattataattggaatctgacaaaaaaaaaaccccactggaaaagtttccataatgtatttcttaaaatagtaaaaattgcataatcaaattatctcaaattaattaagatttatatatgtgagcactttaaatattttatgctatgattattatcagatttcaatgattattttgttcaagctacaaatgtagctatacaaatcactgctaaagtagcagtactggtgtattagtgccaaccaagtttaaataaggaaaataatataagtattcagattattaagatgctgtttttaacaaacaaattttaaattttatgaaaatatgaatttgtaaaggaaacaaacttcattattaatattatggggaaattctgtgaatatatacaatctgacacatgtagaatttgcacattcgatgagaaactgtgtcaaaaatgatcaattgaagcaactcatttaataaaaaagaactctcactaagcaatgctttaatatgttttaaagatataattttaaacacttggaaatcttatatatgtgagtataaaaacacattaaatattcatatcttaatttacttaaaagagttttaacttcaatttattactgaatttaataatcataaacattgggtataaaattacaactttattgttaagcagccagaagagaatgaattctggtaaaatttgataatgattttattgaaaaattgaattaagttcttaaatatgtaacaaccttactagacattaactttataaatgatattaaatgtttataatatattcttttacattctaattctaattaatacttcatacatgtataagtacttaaattttccaaaacacctggtcataatatatatattaattttaattataataaaaatgctaattactttcatacataatatcaaacaatgaaaaaactctaggttggaagcatgtaagagttctcagcattttctagaggaaaaatcaaaagcaaagagaagctaatacctgttctaggcaacatcagaacctatgaattgagaggatgaatggaggctcatggacttcagctatagaaaccaacaaggatggagacaccagaggttttatacccacatttactaccactaaataatcagtatctccctgggaacaccaaagaaacacacataattaccactgaacaaccctaactctcatgggaacacaagagaaatatagtctagtttataaatgtgtaaaatgaagaatagtttattgatgggaatatttcagatattttgttgctcacttatatctacatataagtgcatatgtaataaaaattcatatcccatggcaccattcttgggtggtatagatatgaaacattgctggtttcaaaatgtttaaggattgtgtcaacttttgatgtctgtttactttgaaatatattgtaactgcagaaaagtgcatatacaaggcatatatacacagaattgctgctagttgttatagttttaaacacagttattgtgccatttaacccaggctcaatgaaagttctctgacttgctgataacattttcagggaagaaattacagtgatctttgatcatgaaatgtataattaactcatctgtgtttgactatgccaatagagtgtgtacagcattgtagagaaacagtcttgggattttgtggcagcttttaaaggtagaatcatagcaatgaaaactttggttatgcctgtcctacctaaatgtatacaaaatttgaaccattttcctccattgtgagctgatacgtgaccagatctgcagacatgtaaaattgtgaagttattcccttgtctttaggtatttgcctcaatgcaaattcaaatgtaataattttttttatttctcttaaaatatgtcagttaattatttgattttttctcaattttggtggttttatagaaatgctgaataggctcaatgggttttagatgttttatgtctattattctactctagtctataattatctggatattcattgactcctagctcaaaattagttccttggaaaatttgaagactttccactatttcctcttccccctcatctatttcccattttctcccccatttccatctctttcccccattcctcatcctttatatatatatatatatatatatatacatatatatatatatatatgtgtgtgtgtgtgtgtgtgtgtgtgtgtgtgtgtgtgtgtgtgtacttgcttttttattatcttgtctaatgttgagattgctgtggaaaacctaaaatcatgatcagatggtcacagttctttgatcatcttcttcttcttcttcttcttcttcttcttcttcttcttcttcttcttcttcttcttcttcttcttcttcttcttcttcctcttcctcctcctcctcctcttcctcctcctcttcctcttcgtcgtcttctccttctcctccttcttttccttctcttcctccacctcttcctttcttcttcttttccctcttcttcctccttctcttgtgcctcctctttttgtttttcttacttctctctttgtctctatctctatgtttctgtttctttgtctctgtctctctctctgtgtctctgtctgtcagtctctgtcggcctgtctgtctttctctctgtctcagatgtagattgatctgtatcaattgtactaacccttactagactccttaataaagctactatttttcaattttaagaaaagaccttgaaataatacttcattgtcttcctttcttcatatttcctttgttctctctaaatctgttcttatgacttgtaattacttaactttgaatcttcagtctatttacttttctatttctctatattgttattttaagtctataatctaaaatacttttttagtttctcctatctttgacattgttttaatttctgtaagttactgtttaattcctatgacatcttcacttatatcatagtagttgatcataattgttctttattcccatcattaggaaaagacattgtatattcctgttcttttctatttttttcacctgagttgaaggtattattgagataacagttgagtcatttaccctccagttactcctcccagttcttttcactttctctcccacatgtatctacttcctgtttgtctctcattcaaaaaggaaaggcctctaagagataacagcaaaatttaacaacataaaatataataacataaaacacacaagcaaaaactgtcacatcaaagttgaacaagataaaccaacaaaacaaaaagagcctcaagaaggcagaagaatcagagagcctcttgttcacttacctcagttgaattagaattggttcttatcctgtgggtagtgacccatttagaggtagaatgaccctttcttaaagttttcctaagacaatcagacaatatagatgtttacattatgatttataaatagcaaaattatagttatgaaatagaggcaaaaataattttatggttttgggtcattacaacattaagaaccatatttgagggttctagccttaggaaggttgaaaaccactgaattataaagggccttgtctccttgtgtcctctacccttttgaatctttcacatttctcacctcatcttctgtgggtgggatttagtggagacatcccactgaaagctgtgttccaaatgcatctgtttgttaaatgtcatccgtcaactcttactgttctctttatgatgatgtaaagtagaataaacatcactggcaaatttttgtttatgttaagttgtgacagaatgttcatctaacttgagtaatcctgtaggtgaatatatgtggactacttgatgaataatctgtccaagtacctccgtttggtttttacttcttgggtatattagatttgcagagaggtgtcttttaacttcattcctaatgccttgaaatcctgatcactatatttcagccaccagagtcaagaacttatcactgcaaattctgccctgtgtgtgagtgaataaagcaatttttgtcccatttattctgaaaatttgcgtttaattatatctcacattcttcatactagaatatacattttcctcttttaaaaatatgcacacctgacagactgtgtttgaaatagtgggaacaccatactctatggttccagtgtctgttttgaccatcacattactatcttctctcccaagggccaagtctaccaagtggaatatgcctttaatgctattaaccaggctggtcttatacttgtagctgtcagaggaaaagactgctcaggttttgtcacatagatgaaagtacatgacaaattactagattccaacacacttgttgaagataactgaaaccagtggttgtataatgatgggagtaacagctgacagactccaggtacaaatggcacactatgaggtagttaattggaaagacaaaggtaactatgagtttcctgtggacatgctatgtaaaagaattgctgatattactcaagtcaacacacagaataatgaaatgaggctgcttcattgttgtttgatttttttttactggtataaatgaaaaaacaaggccctcaagtgtactagtggaatcctataggtgttaagccatgacagcagaagttaaatgaatataaccaaccagcttccttgaaaacaataagtgaggaagaaaattgattggatatttgaataaacatttagataatgtaattacatgtctgcctactattctgtcgattggtttcaaactttcagaaatagaaattggaagagttagagttgaaaatactcgattcaggattcttacagaagcagagattggcactcatcttgttgttctagcagagagactgaacattgtcatcagtttaccaaatctgtgatgccacttgcctgtgtgtttgataaaaaccaacatcatagaggctccacagcttaaaatggaacctcttccactcctgccactgagctgcttaggactctgtataaataaaaacagtccttttggaaaaataaatatgtacactgtacttaaaaataaacacatgaaatttttatgtgctacgttaaaacctaactccaaaatttaaagaaacgctacaattgtccacatccattaaaagactccttgttattttatgttctcttttgtaccactattaaattgattctctattgcta

SEQ ID NO: 121 - DNA, 10557 bp, Mus musculus
  source 1..10557: mol_type = other DNA
aaacaggactaatgcttctaagatgattttggatttgggggttggaggatattagaaactaccctctggtaacgaaacgaacagattaccgtttaacatttcttcatcttcatactgctcgcgcggctggacattgtgcttgctggatttttcccggatgctcccagactaacatggatgtcccaccatcccttgcagtggaagcttgctccttggcgcagtgagtgaagaacatgcagagactgctaatgggtttgggaagcggagactccttcctctttctgtgaccatgccgtgattgtgtttgcggccactattccacgcatccttcttctcgtccaagcccggagcctaacgctagatcggggaagtgggtgccgcgcgcgcaggcacggaaacatcatgaagattatttccccagttttaagtaatctagtcttcagtcgctccattaaagtcctgctctgcttgttgtggatcggatattcgcaaggaaccacacatgtgttaagattcggtggtatatttgaatatgtggaatctggccctatgggagctgaagaacttgcattcagatttgctgtgaatacaatcaacaggaacaggactctgctacccaataccacgttaacatatgatacacagaagatcaatctctatgacagttttgaagcatctaagaaagcttgtgatcagctgtctcttggggtggctgccatcttcggtccttcacacagttcatcagcaaatgctgttcagtccatctgcaatgctctgggggttcctcacatacagacccgctggaagcaccaggtgtcagacaataaggattccttctatgtcagtctctacccagacttctcttccctcagccgtgccatcttggatttggtgcagttttttaagtggaaaactgtcacagttgtgtatgacgacagcactggtctcattcgcttgcaagagctcatcaaagctccatcaaggtacaatcttcgacttaaaattcgtcagctgccagctgatacaaaagatgcaaagcctttgctgaaagagatgaagaggggcaaggagttccacgtgatcttcgactgcagccatgaaatggcagcaggcattttaaagcaggcattagctatgggaatgatgacagaatactaccactatatatttacgactctggacctcttcgctcttgatgtggagccctacagatacagtggcgtaaatatgacagggttcagaatactaaatacagagaatacccaagtctcctccatcatcgagaagtggtcgatggaacggttacaggcacctccaaaacctgactcaggtttgctggatggatttatgacgactgatgctgctctgatgtatgatgcagtgcacgttgtgtctgtagctgtccaacagtttccccagatgacagtcagctccttgcaatgcaatcgacacaaaccctggcgctttgggactcgcttcatgagcctaattaaagaggctcattgggaaggtcttacaggcagaattacatttaacaaaaccaatggattgcgaacagattttgatttggatgtgatcagtctcaaggaagaaggtctggagaagattgggacttgggatccatccagtggcctgaatatgacagaaagtcagaaagggaagccagcaaatattacagattcattgtctaatcgttctttgattgttaccaccattttggaagaaccatatgtcctgtttaagaagtctgacaaacctctctatgggaatgatcgatttgaaggctactgtattgatcttctacgagagttatctacaatccttggctttacatatgaaattaggcttgtggaggatgggaaatatggagcccaggatgatgtgaatggacaatggaatggaatggttcgtgagctaattgatcataaagctgaccttgcagttgctccactggctattacctatgttcgtgagaaggtcatcgacttttcaaagccgtttatgactcttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccctatgagtggtataatccacacccttgcaaccctgactcagacgtggtggaaaacaattttaccttgctaaatagtttctggtttggagttggagctctcatgcagcaaggttctgagctcatgcccaaagcactctccaccaggatagtgggaggcatttggtggtttttcacacttatcatcatttcttcgtataccgctaacctagccgcctttctgaccgtggaacgcatggagtcgcctattgactctgctgacgatttagctaagcaaaccaagatagaatatggagcagtagaggacggcgcaaccatgacgtttttcaagaaatcaaaaatctcaacgtatgataaaatgtgggcatttatgagcagcaggagacagtctgtgcttgtcaaaagcaatgaggaagggattcaacgtgtcctcacctccgattatgctttcttaatggagtcaacgaccatcgagtttgttacccagcggaactgtaacctcacgcagattggtggccttatagactccaaaggctatggtgttggcactcccatgggttctccatatcgagacaaaatcaccatagccattcttcagctgcaggaggaaggcaagctgcacatgatgaaggagaagtggtggcgaggcaatggctgcccagaggaggagagcaaagaggccagtgctctaggggtgcagaatattggtggtatcttcattgtcctggcagccggcttggtgctctcagtttttgtggcagtgggagagtttttatacaaatccaaaaaaaacgctcaattggaaaagaggtccttctgtagcgccatggtggaagaactgagaatgtctctgaagtgccagcgtcggctcaaacataagccacaggccccagttattgtgaaaacagaagaagttatcaacatgcacacatttaacgacagaaggttgccaggtaaagaaaccatggcatgaagctgggaggccaatcacccaagcacaaactgtcgtcttttttttttttttcaaacaatttagcgagaatgtttcctgtggaaatatgcaacctgtgcaaaataaaatgagttacctcatgccgctgtgtctatgaactagagactcttgtgatctaagcagtttcagtgatcagacttgatttacaagcaccatggatcgacaaagttacacggggttacactgtttatcatgggttcctcccttcctttgagtgaatgttacatgaaaatgttgtggctggtttcaaatgcagtccagagagaaactgctggttccttctgaagctcaactgttgtcaggagatggaatgttggggcccaaaaggataaccaataaaaatgccataatttataaaagcaaaacaaaaagcgtgtgaaatctgcaaaaattgtagtgtcacaagaaacagtatagtcccatggtcaccaacaaaatgaggtgataatgttactagcccccaatactcagtaaaatcatcatctgaatagataatgtgttcatagaatgtggaaaaaatgtaatgcaaaacatatcagtattcaatcaaagtggaacagaaagcagaccaccatcagttattttcctttctcaatagtctgtgtcatggattgtgatatagatggcaattatctatctaattgttttcttaaaatacccatggcaaatattttaaaatgcaacttgctcccaggaacccctaccctaacctacactagaaataaaaaagccaccactggtataaagattctgatgtaaaagatatgtttttcaatccttgtcatgaattgtaaaacagggctcagtattactggttatatggaagactgaagctttcactctgacattctgatatgtcagctgaaactctccttcctcctggaaaggaccttgatggagcctgggcagattccattgataagactggggacttgtcacctatacagaactacgtgacagaactttgaggtggactgcatttaacaatagtcacaatgttaaaagaacaaaattcttgagcagttttttttttctgttttgttttaaaaaatgttcaggtttatttgtggaaatgcaagatttctatgaaaatagtttttgtatggaaatttttgtaatactttttatcaacaaaataagaacatgtgttcctgtcaggggtgtgatgtcaagcatgaacggtagtgcgtgtgcaccaccaacgtttggtgaaaactatttttatcaagaaaaaggaatcatagaagagaaatattttcaagttagatactataaaagctaggtgcactaccaccacggcttgtcacgccacacccctgagtcccacaaggcagataacatattgtaatgaacagttgtgtgtaaaatgataaaagacacagacctcttgacaacattgtgaaaacagttgagtgcacacagtttgctgtttgaatccaatgcacaaaaattttacaaaactccattaaaattatgtccattttactttcagctttggctttgatttttctcttgcatgtgtaaatgaatgtaacatggtggttttgtatagaaaatatacatcaaggggtcttaggatctcaaagttagaatcttcccaacttacagcaaaaaggaaaaggccatcctggaggtgctcctctctttctctctcccttcctctgtctctctctttgactctgtctctttgtgtctcagtctttctctatatcagtttttctctgtccctctctacctctggctctatcactctctgtctcatttacacacatacacacacacacacacacacacacacacacacgaataaagacatatacattggttttagaattagggtagctggaataaaaagaatatgattgtagagatggcaacctttatcttatctcatttgtagctggaaattgactaagttcactgtgctgcattatgttgtggaatggtaattatctactttggttcaactcatatccaatttcagaattttctgtgcattgatacttcaataatcatcagcagaggaacaaaaagggaaaagtttagaattaataattaattttagatcctaacatattaatagaaacaaactataacagttttacgttttgaaaatcaaatctgtaagattcaacttattttcctgattaattaattaattaattctaagtgtgcaattataattggaatctgacaaaaaaaaaaccccactggaaaagtttccataatgtatttcttaaaatagtaaaaattgcataatcaaattatctcaaattaattaagatttatatatgtgagcactttaaatattttatgctatgattattatcagatttcaatgattattttgttcaagctacaaatgtagctatacaaatcactgctaaagtagcagtactggtgtattagtgccaaccaagtttaaataaggaaaataatataagtattcagattattaagatgctgtttttaacaaacaaattttaaattttatgaaaatatgaatttgtaaaggaaacaaacttcattattaatattatggggaaattctgtgaatatatacaatctgacacatgtagaatttgcacattcgatgagaaactgtgtcaaaaatgatcaattgaagcaactcatttaataaaaaagaactctcactaagcaatgctttaatatgttttaaagatataattttaaacacttggaaatcttatatatgtgagtataaaaacacattaaatattcatatcttaatttacttaaaagagttttaacttcaatttattactgaatttaataatcataaacattgggtataaaattacaactttattgttaagcagccagaagagaatgaattctggtaaaatttgataatgattttattgaaaaattgaattaagttcttaaatatgtaacaaccttactagacattaactttataaatgatattaaatgtttataatatattcttttacattctaattctaattaatacttcatacatgtataagtacttaaattttccaaaacacctggtcataatatatatattaattttaattataataaaaatgctaattactttcatacataatatcaaacaatgaaaaaactctaggttggaagcatgtaagagttctcagcattttctagaggaaaaatcaaaagcaaagagaagctaatacctgttctaggcaacatcagaacctatgaattgagaggatgaatggaggctcatggacttcagctatagaaaccaacaaggatggagacaccagaggttttatacccacatttactaccactaaataatcagtatctccctgggaacaccaaagaaacacacataattaccactgaacaaccctaactctcatgggaacacaagagaaatatagtctagtttataaatgtgtaaaatgaagaatagtttattgatgggaatatttcagatattttgttgctcacttatatctacatataagtgcatatgtaataaaaattcatatcccatggcaccattcttgggtggtatagatatgaaacattgctggtttcaaaatgtttaaggattgtgtcaacttttgatgtctgtttactttgaaatatattgtaactgcagaaaagtgcatatacaaggcatatatacacagaattgctgctagttgttatagttttaaacacagttattgtgccatttaacccaggctcaatgaaagttctctgacttgctgataacattttcagggaagaaattacagtgatctttgatcatgaaatgtataattaactcatctgtgtttgactatgccaatagagtgtgtacagcattgtagagaaacagtcttgggattttgtggcagcttttaaaggtagaatcatagcaatgaaaactttggttatgcctgtcctacctaaatgtatacaaaatttgaaccattttcctccattgtgagctgatacgtgaccagatctgcagacatgtaaaattgtgaagttattcccttgtctttaggtatttgcctcaatgcaaattcaaatgtaataattttttttatttctcttaaaatatgtcagttaattatttgattttttctcaattttggtggttttatagaaatgctgaataggctcaatgggttttagatgttttatgtctattattctactctagtctataattatctggatattcattgactcctagctcaaaattagttccttggaaaatttgaagactttccactatttcctcttccccctcatctatttcccattttctcccccatttccatctctttcccccattcctcatcctttatatatatatatatatatatatatacatatatatatatatatatgtgtgtgtgtgtgtgtgtgtgtgtgtgtgtgtgtgtgtgtgtgtacttgcttttttattatcttgtctaatgttgagattgctgtggaaaacctaaaatcatgatcagatggtcacagttctttgatcatcttcttcttcttcttcttcttcttcttcttcttcttcttcttcttcttcttcttcttcttcttcttcttcttcttcttcctcttcctcctcctcctcctcttcctcctcctcttcctcttcgtcgtcttctccttctcctccttcttttccttctcttcctccacctcttcctttcttcttcttttccctcttcttcctccttctcttgtgcctcctctttttgtttttcttacttctctctttgtctctatctctatgtttctgtttctttgtctctgtctctctctctgtgtctctgtctgtcagtctctgtcggcctgtctgtctttctctctgtctcagatgtagattgatctgtatcaattgtactaacccttactagactccttaataaagctactatttttcaattttaagaaaagaccttgaaataatacttcattgtcttcctttcttcatatttcctttgttctctctaaatctgttcttatgacttgtaattacttaactttgaatcttcagtctatttacttttctatttctctatattgttattttaagtctataatctaaaatacttttttagtttctcctatctttgacattgttttaatttctgtaagttactgtttaattcctatgacatcttcacttatatcatagtagttgatcataattgttctttattcccatcattaggaaaagacattgtatattcctgttcttttctatttttttcacctgagttgaaggtattattgagataacagttgagtcatttaccctccagttactcctcccagttcttttcactttctctcccacatgtatctacttcctgtttgtctctcattcaaaaaggaaaggcctctaagagataacagcaaaatttaacaacataaaatataataacataaaacacacaagcaaaaactgtcacatcaaagttgaacaagataaaccaacaaaacaaaaagagcctcaagaaggcagaagaatcagagagcctcttgttcacttacctcagttgaattagaattggttcttatcctgtgggtagtgacccatttagaggtagaatgaccctttcttaaagttttcctaagacaatcagacaatatagatgtttacattatgatttataaatagcaaaattatagttatgaaatagaggcaaaaataattttatggttttgggtcattacaacattaagaaccatatttgagggttctagccttaggaaggttgaaaaccactgaattataaagggccttgtctccttgtgtcctctacccttttgaatctttcacatttctcacctcatcttctgtgggtgggatttagtggagacatcccactgaaagctgtgttccaaatgcatctgtttgttaaatgtcatccgtcaactcttactgttctctttatgatgatgtaaagtagaataaacatcactggcaaatttttgtttatgttaagttgtgacagaatgttcatctaacttgagtaatcctgtaggtgaatatatgtggactacttgatgaataatctgtccaagtacctccgtttggtttttacttcttgggtatattagatttgcagagaggtgtcttttaacttcattcctaatgccttgaaatcctgatcactatatttcagccaccagagtcaagaacttatcactgcaaattctgccctgtgtgtgagtgaataaagcaatttttgtcccatttattctgaaaatttgcgtttaattatatctcacattcttcatactagaatatacattttcctcttttaaaaatatgcacacctgacagactgtgtttgaaatagtgggaacaccatactctatggttccagtgtctgttttgaccatcacattactatcttctctcccaagggccaagtctaccaagtggaatatgcctttaatgctattaaccaggctggtcttatacttgtagctgtcagaggaaaagactgctcaggttttgtcacatagatgaaagtacatgacaaattactagattccaacacacttgttgaagataactgaaaccagtggttgtataatgatgggagtaacagctgacagactccaggtacaaatggcacactatgaggtagttaattggaaagacaaaggtaactatgagtttcctgtggacatgctatgtaaaagaattgctgatattactcaagtcaacacacagaataatgaaatgaggctgcttcattgttgtttgatttttttttactggtataaatgaaaaaacaaggccctcaagtgtactagtggaatcctataggtgttaagccatgacagcagaagttaaatgaatataaccaaccagcttccttgaaaacaataagtgaggaagaaaattgattggatatttgaataaacatttagataatgtaattacatgtctgcctactattctgtcgattggtttcaaactttcagaaatagaaattggaagagttagagttgaaaatactcgattcaggattcttacagaagcagagattggcactcatcttgttgttctagcagagagactgaacattgtcatcagtttaccaaatctgtgatgccacttgcctgtgtgtttgataaaaaccaacatcatagaggctccacagcttaaaatggaacctcttccactcctgccactgagctgcttaggactctgtataaataaaaacagtccttttggaaaaataaatatgtacactgtacttaaaaataaacacatgaaatttttatgtgctacgttaaaacctaactccaaaatttaaagaaacgctacaattgtccacatccattaaaagactccttgttattttatgttctcttttgtaccactattaaattgattctctattgcta

SEQ ID NO: 122 - DNA, 4579 bp, Macaca mulatta
  source 1..4579: mol_type = other DNA
gctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctgtgaccatgccgtgatcgtgtctgcggtcaccactcgacgcatccttatttctgcccgaacccgggagccgaacgctagatcggggaagtgggtgccgtgcgtgtgggcacagaaacaccatgaagattattttccccattctaagtaatccagtcttcaggcgcaccgttaaactcctgctctgtttactgtggattggatattctcaaggaactacacatgtattaagatttggtggtatttttgaatatgtggaatctggcccaatgggagctgaggaacttgcattcagatttgctgtgaatacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaatgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtacaatcttcgactcaaaattcgtcagctacctgctgatacaaaggatgcaaaacccttactaaaagaaatgaaaagaggcaaggagtttcacgtaatctttgattgtagccatgaaatggcagcaggcattttaaaacaggcattagctatgggaatgatgacagaatactatcattacatctttaccactctggacctctttgctcttgacgttgagccgtaccgatacagtggtgttaatatgacagggttcagaatattaaatacagaaaatacccaagtttcctccatcattgaaaagtggtcaatggaacgattgcaggcacctccgaaacccgattcaggtttgctggatggatttatgacgactgatgctgctctaatgtatgatgctgtgcatgtggtgtctgtggccgtccaacagtttccccagatgacagtcagttccttgcagtgtaatcgacataaaccctggcgcttcgggactcgctttatgagtctaattaaagaggcacattgggaaggcctcacaggcagaataactttcaacaaaaccaatggcttgcgaacagattttgatttggatgtgatcagtctcaaggaagaaggtctagaaaagattggaacgtgggatccagccagtggcctgaatatgacagaaagtcaaaagggaaagccggcaaacatcacagattccttatccaatcgttctttgattgttaccaccattttggaagaaccctatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactccaaaggttatggcgttggcactcccatgggttctccatatcgagacaaaattaccatagcaattcttcagctgcaagaggaaggcaagctgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcagttggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagcgagaatgtttcctgtggaaatatgcaacctgtgcaaaataaaatgagttacctcatgccgctgtgtctatgaactagagactctgtgatctaagcaattgcaatgatcagacttgatttacaagcatcatggatcaaccaagttacacggggttacactgttaatcatgggttcctcccttcttctgagtgaatgttacatgagcattttgtggctggtttcaaatgcagtccagtgagaaattacaggttccttttgaagctcaactgttgccaggagatggaatatcgacgcccaacagggcaaccaatcaaagtgtcactaagaatacaaatatttggaatcagcaaaaactgtagtgttacagaacagtacagtcttctgaacacccagatcatagaggtgatgatgctactagcccccaactactcagtataattattgtctgaatacacagtatatgtttataggatgtgaaaaaatgtaatgcaaaacaaatttgaatcccatggcagttggaatataaagcagatgtttatcacttattttcctttttttcttttcctttttgttttttttttttttgacagtctgtgtctctgattgagatagaaatgccaatttttaaggcaataatgcttttcttaagttccctaaggcagaagatttaacatgcaactctaccatatccctttctattcccccaacaccttttctctaacctccgtatcccaaataataataataatagtaataataaaaacagttggttcagtgattctgaattaaaaggataatgttttgcaatctttaagttgtaaaaagggctgagtattggctgtgtggaagactaaagctttcattctaacattcagacatagcaatccaaaccctgttcctgctgtaaatgaacttgatggagcatgggcagatctcagtggtacgagaaaggggactggtcatctatagaaaaatctgtgagagaacttggacatggactgcatttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagagaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataactgttgagtgcacacagtttgctgtttgaatccaatgcacaaaattaaaaaaaaatcattaaaattatgttcattttactttca

SEQ ID NO: 123 - DNA, 4722 bp, Macaca mulatta
  source 1..4722: mol_type = other DNA
ccgtcttcgcctttgccgtcactcgcgctcggcgccggcggctgcgctggtcggtctgggagccggggactttccgcccgacctcctccggctgctcctccccgaggaccaccccaccccctccccacccacctcacccctagcgccaggctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctgtgaccatgccgtgatcgtgtctgcggtcaccactcgacgcatccttatttctgcccgaacccgggagccgaacgctagatcggggaagtgggtgccgtgcgtgtgggcacagaaacaccatgaagattattttccccattctaagtaatccagtcttcaggcgcaccgttaaactcctgctctgtttactgtggattggatattctcaaggaactacacatgtattaagatttggtggtatttttgaatatgtggaatctggcccaatgggagctgaggaacttgcattcagatttgctgtgaatacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaatgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtacaatcttcgactcaaaattcgtcagctacctgctgatacaaaggatgcaaaacccttactaaaagaaatgaaaagaggcaaggagtttcacgtaatctttgattgtagccatgaaatggcagcaggcattttaaaacaggcattagctatgggaatgatgacagaatactatcattacatctttaccactctggacctctttgctcttgacgttgagccgtaccgatacagtggtgttaatatgacagggttcagaatattaaatacagaaaatacccaagtttcctccatcattgaaaagtggtcaatggaacgattgcaggcacctccgaaacccgattcaggtttgctggatggatttatgacgactgatgctgctctaatgtatgatgctgtgcatgtggtgtctgtggccgtccaacagtttccccagatgacagtcagttccttgcagtgtaatcgacataaaccctggcgcttcgggactcgctttatgagtctaattaaagaggcacattgggaaggcctcacaggcagaataactttcaacaaaaccaatggcttgcgaacagattttgatttggatgtgatcagtctcaaggaagaaggtctagaaaagattggaacgtgggatccagccagtggcctgaatatgacagaaagtcaaaagggaaagccggcaaacatcacagattccttatccaatcgttctttgattgttaccaccattttggaagaaccctatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactccaaaggttatggcgttggcactcccatgggttctccatatcgagacaaaattaccatagcaattcttcagctgcaagaggaaggcaagctgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcagttggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagcgagaatgtttcctgtggaaatatgcaacctgtgcaaaataaaatgagttacctcatgccgctgtgtctatgaactagagactctgtgatctaagcaattgcaatgatcagacttgatttacaagcatcatggatcaaccaagttacacggggttacactgttaatcatgggttcctcccttcttctgagtgaatgttacatgagcattttgtggctggtttcaaatgcagtccagtgagaaattacaggttccttttgaagctcaactgttgccaggagatggaatatcgacgcccaacagggcaaccaatcaaagtgtcactaagaatacaaatatttggaatcagcaaaaactgtagtgttacagaacagtacagtcttctgaacacccagatcatagaggtgatgatgctactagcccccaactactcagtataattattgtctgaatacacagtatatgtttataggatgtgaaaaaatgtaatgcaaaacaaatttgaatcccatggcagttggaatataaagcagatgtttatcacttattttcctttttttcttttcctttttgttttttttttttttgacagtctgtgtctctgattgagatagaaatgccaatttttaaggcaataatgcttttcttaagttccctaaggcagaagatttaacatgcaactctaccatatccctttctattcccccaacaccttttctctaacctccgtatcccaaataataataataatagtaataataaaaacagttggttcagtgattctgaattaaaaggataatgttttgcaatctttaagttgtaaaaagggctgagtattggctgtgtggaagactaaagctttcattctaacattcagacatagcaatccaaaccctgttcctgctgtaaatgaacttgatggagcatgggcagatctcagtggtacgagaaaggggactggtcatctatagaaaaatctgtgagagaacttggacatggactgcatttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagagaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataactgttgagtgcacacagtttgctgtttgaatccaatgcacaaaattaaaaaaaaatcattaaaattatgttcatttta

SEQ ID NO: 124 - DNA, 3058 bp, Rattus norvegicus
  source 1..3058: mol_type = other DNA
ggctggacattgtgcttgctggatttttcccggatgctcccggactaacatggatgtcccaccatcccttgcagtggaagcttgctccttggcgcagtgagagtgaagaacatgcagcgactgctaatgggtttgggaagcggagactccttcctctttctgtgaccatgccgtgattgtgtctgcggccactactccacgcatcttccttctcgtccaagcccggagcctaacgctagatcggggaagtgggtgccgcgcgcgcaggcacggaaacatcatgaagattatttccccagttttaagtaatctagtcttcagtcgctccattaaagtcctgctctgcttattgtggatcggatattcgcaaggaaccacacatgtgttaagattcggtggtatatttgaatatgtggaatctggccccatgggagcagaagaacttgcattcagatttgctgtgaataccatcaacagaaacaggactttgctgcccaacaccactttaacttatgatactcagaagatcaatctctatgacagttttgaagcatctaagaaagcttgtgatcagctgtctcttggggtggctgctatcttcggtccttcacacagttcatcagccaatgctgtgcagtccatctgcaatgctctgggggttccccacatacagacccgctggaagcaccaggtgtcagacaacaaggattccttctacgtcagtctctacccagacttctcttccctgagccgcgccatcttggatttggtgcagttttttaagtggaaaactgtcacagttgtgtatgacgacagcactggtctcattcgcttgcaagagctcatcaaagctccatcgaggtacaatcttcgacttaaaattcgtcagctgccagctgataccaaagatgcaaaacctttgctgaaggagatgaaaagaggcaaggagttccacgtgatcttcgactgcagccatgagatggcagcaggcattttaaaacaggcattagctatgggaatgatgacagaatactatcactatatatttacaactctggacctctttgctcttgacgtggagccctacagatacagtggcgtaaatatgacagggttcaggatactaaatacagagaatacccaagtctcctccatcatcgaaaagtggtctatggaacggttacaggcgcctccaaaacctgactcaggtttgctggatggatttatgacgactgatgctgctctgatgtatgatgcagtgcacgttgtgtctgtggctgtccaacagtttccccagatgacagtcagctccttgcaatgcaatcgacacaaaccctggcgctttgggacccgcttcatgagtctaattaaagaggctcactgggaaggtctcacaggcagaataacatttaacaaaaccaatggattacggacagattttgatttggatgtgatcagtctcaaggaagaaggtctggagaagattggaacttgggatccagccagtggcctgaatatgacagaaagtcagaaaggaaagccagcaaatatcacagactcattgtctaatcgttctttgattgttaccaccattttggaagaaccgtatgttctgtttaagaagtctgacaaaccactctatgggaatgatcgatttgaaggctactgtattgatctcctacgagagttatctacaatccttggctttacatatgagattaggcttgtggaggatgggaaatatggagcccaggatgatgtgaacggacaatggaatggaatggttcgtgaactaatcgatcataaagctgaccttgcagttgctccactggctataacctatgttcgtgagaaggtcatcgacttttcaaagccgtttatgacacttggaataagtattttgtaccgcaagcccaatggtacaaacccaggcgtcttctccttcctgaatcctctctcccctgatatctggatgtatgttctgctggcttgcttgggtgtcagttgtgtgctctttgtcatagccaggtttagtccctatgagtggtataacccacacccttgcaaccctgactcagacgtggtggaaaacaattttaccttgctaaatagtttctggtttggagttggagctctcatgcggcaaggttctgagctcatgcccaaagcactctccaccaggatagtgggaggcatttggtggtttttcacacttatcatcatttcttcgtataccgctaacctagccgcctttctgactgtggaacgcatggagtcgcccattgactctgctgacgatttagctaagcaaaccaagatagagtatggagcagtggaggacggcgcaaccatgacgttttttaagaaatcaaaaatttcaacgtatgataaaatgtgggcgtttatgagcagcaggagacagtctgtgcttgtcaaaagcaatgaggaagggatccaacgagtcctcacctcggattatgctttcttaatggagtcaacaaccatcgagtttgttacacagcggaactgtaacctcacgcagattggcggccttatagactccaaaggctatggcgttggcactcctatgggctctccatatcgagacaaaatcaccatagcaattcttcagctgcaggaggaaggcaagctgcacatgatgaaggagaaatggtggcggggcaatggctgcccagaggaggagagcaaagaggccagtgctctgggggtgcagaatattggtggtatcttcattgtcctggcagccggcttggtgctctcagtttttgtggcagtgggagagtttttatacaaatccaaaaaaaacgctcaattggaaaagaggtccttctgtagcgctatggtggaagagctgagaatgtccctgaagtgccagcgtcggctcaaacataagccacaggccccagttattgtgaaaacagaagaagttatcaacatgcacacatttaacgacagaaggttgccaggtaaagaaaccatggcatgaagctgggaggccaatcacccaagcacaaactgtcgtctttttttttttttt

SEQ ID NO: 125 - DNA, 2631 bp, Homo sapiens
  source 1..2631: mol_type = other DNA
accacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggca

SEQ ID NO: 126 - DNA, 293 bp, Homo sapiens
  source 1..293: mol_type = other DNA
gctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctatgaccatgccgtgatcgtgtctgcggtcaccactcgacgcatcctcatttctacccgaacccaggagccgaacgctagatcggggaagtgggtgccgtgcgtgtgggcacagaaacacc

SEQ ID NO: 127 - DNA, 1572 bp, Homo sapiens
  source 1..1572: mol_type = other DNA
agctgggaggccaaacacccaagcacaaactgtcgtctttttccaaacaatttagccagaatgtttcctgtggaaatatgcaacctgtgcaaaataaaatgagttacctcatgccgctgtgtctatgaactagagactctgtgatctaagcagttgcaatgatcagacttgatttacaagcatcatggatcaaccaagttacacggggttacactgttaatcatgggttcctcccttcttctgagtgaatgttaacatgcgcattttgtggctgatttcaaatgcagtccagtgagaaattacaggttccttttgaagctcaactgttgccaggagatggaatatcaatgcccaacagggcaaccaataaaagtgtcactaagaatataaatatttggaatcagcaaaaactgtagtgttacaggaaacagtacagtcttctgaacacccagatcatagaggtgatgatgttactagcccccaactactcagtataattattgtctgaatgcaaagtatgtgtttataggatgtgaaaaaatgtaatgcaaaacaaatttgaatcccatggcagttggaatataaagcagatgttcatcacttattttccttttttcttttcttatttttttttttgacagtctgtgtcactgattgagatagaaatgccaattatcaaggaaataatgttttcttaagttccctaaggcagaagatttaacatgcaattctaccagatcccttcctattcccccaacaccttttctctaacccccatatcccaaataataataataataataataataataataataataataataaaagcagttggttcagtgattctgaattaaaaggataatgttttgcaatgttcaagttgtaaaaactggccgagtattggctgtgtggaagactaaagctttcattctaacattcagacatagcaatccaaacccttgttcctgctgtaaatgaacttgatggagcatgggcagatttcagtgatacgagaaaggggactggtcatctatagaaaaatctgtgagagaacttggaagtggactgcgtttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagaaaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataacagttgagtgcacacagtttgctgtttgaatccaatgcacaaaattaaaaaaaatcattaaaactatgttcattttactttca

SEQ ID NO: 128 - DNA, 93 bp, Homo sapiens
  source 1..93: mol_type = other DNA
atgaagattattttcccgattctaagtaatccagtcttcaggcgcaccgttaaactcctgctctgtttactgtggattggatattctcaagga

SEQ ID NO: 129 - DNA, 408 bp, Homo sapiens
  source 1..408: mol_type = other DNA
gctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctatgaccatgccgtgatcgtgtctgcggtcaccactcgacgcatcctcatttctacccgaacccaggagccgaacgctagatcggggaagtgggtgccgtgcgtgtgggcacagaaacaccatgaagattattttcccgattctaagtaatccagtcttcaggcgcaccgttaaactcctgctctgtttactgtggattggatattctcaaggaaccacacatgtattaagatttg

SEQ ID NO: 130 - DNA, 168 bp, Homo sapiens
  source 1..168: mol_type = other DNA
gtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaag

SEQ ID NO: 131 - DNA, 258 bp, Homo sapiens
  source 1..258: mol_type = other DNA
cctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactg

SEQ ID NO: 132 - DNA, 182 bp, Homo sapiens
  source 1..182: mol_type = other DNA
gtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacag

SEQ ID NO: 133 - DNA, 54 bp, Homo sapiens
  source 1..54: mol_type = other DNA
gcattagctatgggaatgatgacagaatactatcattatatctttaccactctg

SEQ ID NO: 134 - DNA, 174 bp, Homo sapiens
  source 1..174: mol_type = other DNA
gacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacg

SEQ ID NO: 135 - DNA, 144 bp, Homo sapiens
  source 1..144: mol_type = other DNA
actgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagag

SEQ ID NO: 136 - DNA, 108 bp, Homo sapiens
  source 1..108: mol_type = other DNA
gcacattgggaaggcctcacaggcagaataactttcaacaaaaccaatggcttgagaacagattttgatttggatgtgatcagtctgaaggaagaaggtctagaaaag

SEQ ID NO: 137 - DNA, 114 bp, Homo sapiens
  source 1..114: mol_type = other DNA
attggaacgtgggatccagccagtggcctgaatatgacagaaagtcaaaagggaaagccagcgaacatcacagattccttatccaatcgttctttgattgttaccaccattttg

SEQ ID NO: 138 - DNA, 207 bp, Homo sapiens
  source 1..207: mol_type = other DNA
gaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcat

SEQ ID NO: 139 - DNA, 224 bp, Homo sapiens
  source 1..224: mol_type = other DNA
aaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccag

SEQ ID NO: 140 - DNA, 119 bp, Homo sapiens
  source 1..119: mol_type = other DNA
gtttagtccttatgagtggtataatccacacccttgcaaccctgactcagacgtggtggaaaacaattttaccttgctaaatagtttctggtttggagttggagctctcatgcagcaag

SEQ ID NO: 141 - DNA, 218 bp, Homo sapiens
  source 1..218: mol_type = other DNA
gttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaag

SEQ ID NO: 142 - DNA, 226 bp, Homo sapiens
  source 1..226: mol_type = other DNA
aaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgg

SEQ ID NO: 143 - DNA, 251 bp, Homo sapiens
  source 1..251: mol_type = other DNA
gttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaag

SEQ ID NO: 144 - DNA, 1737 bp, Homo sapiens
  source 1..1737: mol_type = other DNA
aggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagccagaatgtttcctgtggaaatatgcaacctgtgcaaaataaaatgagttacctcatgccgctgtgtctatgaactagagactctgtgatctaagcagttgcaatgatcagacttgatttacaagcatcatggatcaaccaagttacacggggttacactgttaatcatgggttcctcccttcttctgagtgaatgttaacatgcgcattttgtggctgatttcaaatgcagtccagtgagaaattacaggttccttttgaagctcaactgttgccaggagatggaatatcaatgcccaacagggcaaccaataaaagtgtcactaagaatataaatatttggaatcagcaaaaactgtagtgttacaggaaacagtacagtcttctgaacacccagatcatagaggtgatgatgttactagcccccaactactcagtataattattgtctgaatgcaaagtatgtgtttataggatgtgaaaaaatgtaatgcaaaacaaatttgaatcccatggcagttggaatataaagcagatgttcatcacttattttccttttttcttttcttatttttttttttgacagtctgtgtcactgattgagatagaaatgccaattatcaaggaaataatgttttcttaagttccctaaggcagaagatttaacatgcaattctaccagatcccttcctattcccccaacaccttttctctaacccccatatcccaaataataataataataataataataataataataataataataaaagcagttggttcagtgattctgaattaaaaggataatgttttgcaatgttcaagttgtaaaaactggccgagtattggctgtgtggaagactaaagctttcattctaacattcagacatagcaatccaaacccttgttcctgctgtaaatgaacttgatggagcatgggcagatttcagtgatacgagaaaggggactggtcatctatagaaaaatctgtgagagaacttggaagtggactgcgtttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagaaaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataacagttgagtgcacacagtttgctgtttgaatccaatgcacaaaattaaaaaaaatcattaaaactatgttcattttactttca

SEQ ID NO: 145 - DNA, 31 bp, Homo sapiens
  source 1..31: mol_type = other DNA
cagaacattgctacccaatactacccttacc

SEQ ID NO: 146 - DNA, 27 bp, Homo sapiens
  source 1..27: mol_type = other DNA
taccgatacagtggtgttaacatgaca

SEQ ID NO: 147 - DNA, 41 bp, Homo sapiens
  source 1..41: mol_type = other DNA
caggcacctccgaaacccgattcaggtttgctggatggatt

SEQ ID NO: 148 - DNA, 27 bp, Homo sapiens
  source 1..27: mol_type = other DNA
aaaagggaaagccagcgaacatcacag

SEQ ID NO: 149 - DNA, 65 bp, Homo sapiens
  source 1..65: mol_type = other DNA
atttgaaggctattgcattgatctcctcagagagttatctacaatccttggctttacatatgaaa

SEQ ID NO: 150 - DNA, 64 bp, Homo sapiens
  source 1..64: mol_type = other DNA
gatgatgccaatggacaatggaatggaatggttcgtgaactaattgatcataaagctgaccttg

SEQ ID NO: 151 - DNA, 24 bp, Homo sapiens
  source 1..24: mol_type = other DNA
gggcaatggttgcccagaagagga

SEQ ID NO: 152 - DNA, 40 bp, Homo sapiens
  source 1..40: mol_type = other DNA
ttaaaacataagccacaggccccagttattgtgaaaacag

SEQ ID NO: 153 - DNA, 27 bp, Homo sapiens
  source 1..27: mol_type = other DNA
aaacaatttagccagaatgtttcctgt

SEQ ID NO: 154 - DNA, 23 bp, Homo sapiens
  source 1..23: mol_type = other DNA
gtctatgaactagagactctgtg

SEQ ID NO: 155 - DNA, 32 bp, Homo sapiens
  source 1..32: mol_type = other DNA
aaccaataaaagtgtcactaagaatataaata

SEQ ID NO: 156 - DNA, 69 bp, Homo sapiens
  source 1..69: mol_type = other DNA
ctaacccccatatcccaaataataataataataataataataataataataataataataaaagcagtt

SEQ ID NO: 157 - DNA, 43 bp, Homo sapiens
  source 1..43: mol_type = other DNA
aaaattaaaaaaaatcattaaaactatgttcattttactttca

SEQ ID NO: 158 - DNA, 24 bp, Homo sapiens
  source 1..24: mol_type = other DNA
caagttagataatataaaagctag

SEQ ID NO: 159 - DNA, 79 bp, Homo sapiens
  source 1..79: mol_type = other DNA
tttggtggtttttcacacttatcatcatttcttcgtatactgctaacttagccgcctttctgacagtggaacgcatgga

SEQ ID NO: 160 - DNA, 37 bp, Homo sapiens
  source 1..37: mol_type = other DNA
ggtgcaaccatgacttttttcaagaaatcaaaaatct

SEQ ID NO: 161 - DNA, 45 bp, Homo sapiens
  source 1..45: mol_type = other DNA
aggttccttttgaagctcaactgttgccaggagatggaatatcaa

SEQ ID NO: 162 - DNA, 53 bp, Homo sapiens
  source 1..53: mol_type = other DNA
catggcagttggaatataaagcagatgttcatcacttattttccttttttctt

SEQ ID NO: 163 - DNA, 28 bp, Homo sapiens
  source 1..28: mol_type = other DNA
atgccaattatcaaggaaataatgtttt

SEQ ID NO: 164 - DNA, 20 bp, Homo sapiens
  source 1..20: mol_type = other DNA
cagaacattgctacccaata

SEQ ID NO: 165 - DNA, 20 bp, Homo sapiens
  source 1..20: mol_type = other DNA
agaacattgctacccaatac

SEQ ID NO: 166 - DNA, 20 bp, Homo sapiens
  source 1..20: mol_type = other DNA
gaacattgctacccaatact

SEQ ID NO: 167 - DNA, 20 bp, Homo sapiens
  source 1..20: mol_type = other DNA
aacattgctacccaatacta

SEQ ID NO: 168 - DNA, 20 bp, Homo sapiens
  source 1..20: mol_type = other DNA
acattgctacccaatactac

SEQ ID NO: 169 - DNA, 20 bp, Homo sapiens
  source 1..20: mol_type = other DNA
cattgctacccaatactacc

SEQ ID NO: 170 - DNA, 20 bp, Homo sapiens
  source 1..20: mol_type = other DNA
attgctacccaatactaccc

SEQ ID NO: 171 - DNA, 20 bp, Homo sapiens
  source 1..20: mol_type = other DNA
ttgctacccaatactaccct

SEQ ID NO: 172 - DNA, 20 bp, Homo sapiens
  source 1..20: mol_type = other DNA
tgctacccaatactaccctt

SEQ ID NO: 173 - DNA, 20 bp, Homo sapiens
  source 1..20: mol_type = other DNA
gctacccaatactaccctta

SEQ ID NO: 174 - DNA, 20 bp, Homo sapiens
  source 1..20: mol_type = other DNA
ctacccaatactacccttac

SEQ ID NO: 175 - DNA, 20 bp, Homo sapiens
  source 1..20: mol_type = other DNA
tacccaatactacccttacc

SEQ ID NO: 176 - DNA, 20 bp, Homo sapiens
  source 1..20: mol_type = other DNA
taccgatacagtggtgttaa

SEQ ID NO: 177 - DNA, 20 bp, Homo sapiens
  source 1..20: mol_type = other DNA
accgatacagtggtgttaac

SEQ ID NO: 178 - DNA, 20 bp, Homo sapiens
  source 1..20: mol_type = other DNA
ccgatacagtggtgttaaca

SEQ ID NO: 179 - DNA, 20 bp, Homo sapiens
  source 1..20: mol_type = other DNA
cgatacagtggtgttaacat

SEQ ID NO: 180 - DNA, 20 bp, Homo sapiens
  source 1..20: mol_type = other DNA
gatacagtggtgttaacatg

SEQ ID NO: 181 - DNA, 20 bp, Homo sapiens
  source 1..20: mol_type = other DNA
atacagtggtgttaacatga

SEQ ID NO: 182 - DNA, 20 bp, Homo sapiens
  source 1..20: mol_type = other DNA
tacagtggtgttaacatgac

SEQ ID NO: 183 - DNA, 20 bp, Homo sapiens
  source 1..20: mol_type = other DNA
acagtggtgttaacatgaca

SEQ ID NO: 184 - DNA, 20 bp, Homo sapiens
  source 1..20: mol_type = other DNA
caggcacctccgaaacccga

SEQ ID NO: 185 - DNA, 20 bp, Homo sapiens
  source 1..20: mol_type = other DNA
aggcacctccgaaacccgat

SEQ ID NO: 186 - DNA, 20 bp, Homo sapiens
  source 1..20: mol_type = other DNA
ggcacctccgaaacccgatt

SEQ ID NO: 187 - DNA, 20 bp, Homo sapiens
  source 1..20: mol_type = other DNA
gcacctccgaaacccgattc

SEQ ID NO: 188 - DNA, 20 bp, Homo sapiens
  source 1..20: mol_type = other DNA
cacctccgaaacccgattca

SEQ ID NO: 189 - DNA, 20 bp, Homo sapiens
  source 1..20: mol_type = other DNA
acctccgaaacccgattcag

SEQ ID NO: 190 - DNA, 20 bp, Homo sapiens
  source 1..20: mol_type = other DNA
cctccgaaacccgattcagg

SEQ ID NO: 191 - DNA, 20 bp, Homo sapiens
  source 1..20: mol_type = other DNA
ctccgaaacccgattcaggt

SEQ ID NO: 192 - DNA, 20 bp, Homo sapiens
  source 1..20: mol_type = other DNA
tccgaaacccgattcaggtt

SEQ ID NO: 193 - DNA, 20 bp, Homo sapiens
  source 1..20: mol_type = other DNA
ccgaaacccgattcaggttt

SEQ ID NO: 194 - DNA, 20 bp, Homo sapiens
  source 1..20: mol_type = other DNA
cgaaacccgattcaggtttg

SEQ ID NO: 195 - DNA, 20 bp, Homo sapiens
  source 1..20: mol_type = other DNA
gaaacccgattcaggtttgc

SEQ ID NO: 196 - DNA, 20 bp, Homo sapiens
  source 1..20: mol_type = other DNA
aaacccgattcaggtttgct

SEQ ID NO: 197 - DNA, 20 bp, Homo sapiens
  source 1..20: mol_type = other DNA
aacccgattcaggtttgctg

SEQ ID NO: 198 - DNA, 20 bp, Homo sapiens
  source 1..20: mol_type = other DNA
acccgattcaggtttgctgg

SEQ ID NO: 199 - DNA, 20 bp, Homo sapiens
  source 1..20: mol_type = other DNA
cccgattcaggtttgctgga

SEQ ID NO: 200 - DNA, 20 bp, Homo sapiens
  source 1..20: mol_type = other DNA
ccgattcaggtttgctggat

SEQ ID NO: 201 - DNA, 20 bp, Homo sapiens
  source 1..20: mol_type = other DNA
cgattcaggtttgctggatg

SEQ ID NO: 202 - DNA, 20 bp, Homo sapiens
  source 1..20: mol_type = other DNA
gattcaggtttgctggatgg

SEQ ID NO: 203 - DNA, 20 bp, Homo sapiens
  source 1..20: mol_type = other DNA
attcaggtttgctggatgga

SEQ ID NO: 204 - DNA, 20 bp, Homo sapiens
  source 1..20: mol_type = other DNA
ttcaggtttgctggatggat

SEQ ID NO: 205 - DNA, 20 bp, Homo sapiens
  source 1..20: mol_type = other DNA
tcaggtttgctggatggatt

SEQ ID NO: 206 - DNA, 20 bp, Homo sapiens
  source 1..20: mol_type = other DNA
aaaagggaaagccagcgaac

SEQ ID NO: 207 - DNA, 20 bp, Homo sapiens
  source 1..20: mol_type = other DNA
aaagggaaagccagcgaaca

SEQ ID NO: 208 - DNA, 20 bp, Homo sapiens
  source 1..20: mol_type = other DNA
aagggaaagccagcgaacat

SEQ ID NO: 209 - DNA, 20 bp, Homo sapiens
  source 1..20: mol_type = other DNA
agggaaagccagcgaacatc

SEQ ID NO: 210 - DNA, 20 bp, Homo sapiens
  source 1..20: mol_type = other DNA
gggaaagccagcgaacatca

SEQ ID NO: 211 - DNA, 20 bp, Homo sapiens
  source 1..20: mol_type = other DNA
ggaaagccagcgaacatcac

SEQ ID NO: 212 - DNA, 20 bp, Homo sapiens
  source 1..20: mol_type = other DNA
gaaagccagcgaacatcaca

SEQ ID NO: 213 - DNA, 20 bp, Homo sapiens
  source 1..20: mol_type = other DNA
aaagccagcgaacatcacag

SEQ ID NO: 214 - DNA, 20 bp, Homo sapiens
  source 1..20: mol_type = other DNA
atttgaaggctattgcattg

SEQ ID NO: 215 - DNA, 20 bp, Homo sapiens
  source 1..20: mol_type = other DNA
tttgaaggctattgcattga

SEQ ID NO: 216 - DNA, 20 bp, Homo sapiens
  source 1..20: mol_type = other DNA
ttgaaggctattgcattgat

SEQ ID NO: 217 - DNA, 20 bp, Homo sapiens
  source 1..20: mol_type = other DNA
tgaaggctattgcattgatc

SEQ ID NO: 218 - DNA, 20 bp, Homo sapiens
  source 1..20: mol_type = other DNA
gaaggctattgcattgatct

SEQ ID NO: 219 - DNA, 20 bp, Homo sapiens
  source 1..20: mol_type = other DNA
aaggctattgcattgatctc

SEQ ID NO: 220 - DNA, 20 bp, Homo sapiens
  source 1..20: mol_type = other DNA
aggctattgcattgatctcc

SEQ ID NO: 221 - DNA, 20 bp, Homo sapiens
  source 1..20: mol_type = other DNA
ggctattgcattgatctcct

SEQ ID NO: 222 - DNA, 20 bp, Homo sapiens
  source 1..20: mol_type = other DNA
gctattgcattgatctcctc

SEQ ID NO: 223 - DNA, 20 bp, Homo sapiens
  source 1..20: mol_type = other DNA
ctattgcattgatctcctca

SEQ ID NO: 224 - DNA, 20 bp, Homo sapiens
  source 1..20: mol_type = other DNA
tattgcattgatctcctcag

SEQ ID NO: 225 - DNA, 20 bp, Homo sapiens
  source 1..20: mol_type = other DNA
attgcattgatctcctcaga

SEQ ID NO: 226 - DNA, 20 bp, Homo sapiens
  source 1..20: mol_type = other DNA
ttgcattgatctcctcagag

SEQ ID NO: 227 - DNA, 20 bp, Homo sapiens
  source 1..20: mol_type = other DNA
tgcattgatctcctcagaga

SEQ ID NO: 228 - DNA, 20 bp, Homo sapiens
  source 1..20: mol_type = other DNA
gcattgatctcctcagagag

SEQ ID NO: 229 - DNA, 20 bp, Homo sapiens
  source 1..20: mol_type = other DNA
cattgatctcctcagagagt

SEQ ID NO: 230 - DNA, 20 bp, Homo sapiens
  source 1..20: mol_type = other DNA
attgatctcctcagagagtt

SEQ ID NO: 231 - DNA, 20 bp, Homo sapiens
  source 1..20: mol_type = other DNA
ttgatctcctcagagagtta

SEQ ID NO: 232 - DNA, 20 bp, Homo sapiens
  source 1..20: mol_type = other DNA
tgatctcctcagagagttat

SEQ ID NO: 233 - DNA, 20 bp, Homo sapiens
  source 1..20: mol_type = other DNA
gatctcctcagagagttatc

SEQ ID NO: 234 - DNA, 20 bp, Homo sapiens
  source 1..20: mol_type = other DNA
atctcctcagagagttatct

SEQ ID NO: 235 - DNA, 20 bp, Homo sapiens
  source 1..20: mol_type = other DNA
tctcctcagagagttatcta

SEQ ID NO: 236 - DNA, 20 bp, Homo sapiens
  source 1..20: mol_type = other DNA
ctcctcagagagttatctac

SEQ ID NO: 237 - DNA, 20 bp, Homo sapiens
  source 1..20: mol_type = other DNA
tcctcagagagttatctaca

SEQ ID NO: 238 - DNA, 20 bp, Homo sapiens
  source 1..20: mol_type = other DNA
cctcagagagttatctacaa

SEQ ID NO: 239 - DNA, 20 bp, Homo sapiens
  source 1..20: mol_type = other DNA
ctcagagagttatctacaat

SEQ ID NO: 240 - DNA, 20 bp, Homo sapiens
  source 1..20: mol_type = other DNA
tcagagagttatctacaatc

SEQ ID NO: 241 - DNA, 20 bp, Homo sapiens
  source 1..20: mol_type = other DNA
cagagagttatctacaatcc

SEQ ID NO: 242 - DNA, 20 bp, Homo sapiens
  source 1..20: mol_type = other DNA
agagagttatctacaatcct

SEQ ID NO: 243 - DNA, 20 bp, Homo sapiens
  source 1..20: mol_type = other DNA
gagagttatctacaatcctt

SEQ ID NO: 244 - DNA, 20 bp, Homo sapiens
  source 1..20: mol_type = other DNA
agagttatctacaatccttg

SEQ ID NO: 245 - DNA, 20 bp, Homo sapiens
  source 1..20: mol_type = other DNA
gagttatctacaatccttgg

SEQ ID NO: 246 - DNA, 20 bp, Homo sapiens
  source 1..20: mol_type = other DNA
agttatctacaatccttggc

SEQ ID NO: 247 - DNA, 20 bp, Homo sapiens
  source 1..20: mol_type = other DNA
gttatctacaatccttggct

SEQ ID NO: 248 - DNA, 20 bp, Homo sapiens
  source 1..20: mol_type = other DNA
ttatctacaatccttggctt

SEQ ID NO: 249 - DNA, 20 bp, Homo sapiens
  source 1..20: mol_type = other DNA
tatctacaatccttggcttt

SEQ ID NO: 250 - DNA, 20 bp, Homo sapiens
  source 1..20: mol_type = other DNA
atctacaatccttggcttta

SEQ ID NO: 251 - DNA, 20 bp, Homo sapiens
  source 1..20: mol_type = other DNA
tctacaatccttggctttac

SEQ ID NO: 252 - DNA, 20 bp, Homo sapiens
  source 1..20: mol_type = other DNA
ctacaatccttggctttaca

SEQ ID NO: 253 - DNA, 20 bp, Homo sapiens
  source 1..20: mol_type = other DNA
tacaatccttggctttacat

SEQ ID NO: 254 - DNA, 20 bp, Homo sapiens
  source 1..20: mol_type = other DNA
acaatccttggctttacata

SEQ ID NO: 255 - DNA, 20 bp, Homo sapiens
  source 1..20: mol_type = other DNA
caatccttggctttacatat

SEQ ID NO: 256 - DNA, 20 bp, Homo sapiens
  source 1..20: mol_type = other DNA
aatccttggctttacatatg

SEQ ID NO: 257 - DNA, 20 bp, Homo sapiens
  source 1..20: mol_type = other DNA
atccttggctttacatatga

SEQ ID NO: 258 - DNA, 20 bp, Homo sapiens
  source 1..20: mol_type = other DNA
tccttggctttacatatgaa

SEQ ID NO: 259 - DNA, 20 bp, Homo sapiens
  source 1..20: mol_type = other DNA
ccttggctttacatatgaaa

SEQ ID NO: 260 - DNA, 20 bp, Homo sapiens
  source 1..20: mol_type = other DNA
gatgatgccaatggacaatg

SEQ ID NO: 261 - DNA, 20 bp, Homo sapiens
  source 1..20: mol_type = other DNA
atgatgccaatggacaatgg

SEQ ID NO: 262 - DNA, 20 bp, Homo sapiens
  source 1..20: mol_type = other DNA
tgatgccaatggacaatgga

SEQ ID NO: 263 - DNA, 20 bp, Homo sapiens
  source 1..20: mol_type = other DNA
gatgccaatggacaatggaa

SEQ ID NO: 264 - DNA, 20 bp, Homo sapiens
  source 1..20: mol_type = other DNA
atgccaatggacaatggaat

SEQ ID NO: 265 - DNA, 20 bp, Homo sapiens
  source 1..20: mol_type = other DNA
tgccaatggacaatggaatg

SEQ ID NO: 266 - DNA, 20 bp, Homo sapiens
  source 1..20: mol_type = other DNA
gccaatggacaatggaatgg

SEQ ID NO: 267 - DNA, 20 bp, Homo sapiens
  source 1..20: mol_type = other DNA
ccaatggacaatggaatgga

SEQ ID NO: 268 - DNA, 20 bp, Homo sapiens
  source 1..20: mol_type = other DNA
caatggacaatggaatggaa

SEQ ID NO: 269 - DNA, 20 bp, Homo sapiens
  source 1..20: mol_type = other DNA
aatggacaatggaatggaat

SEQ ID NO: 270 - DNA, 20 bp, Homo sapiens
  source 1..20: mol_type = other DNA
atggacaatggaatggaatg

SEQ ID NO: 271 - DNA, 20 bp, Homo sapiens
  source 1..20: mol_type = other DNA
tggacaatggaatggaatgg

SEQ ID NO: 272 - DNA, 20 bp, Homo sapiens
  source 1..20: mol_type = other DNA
ggacaatggaatggaatggt

SEQ ID NO: 273 - DNA, 20 bp, Homo sapiens
  source 1..20: mol_type = other DNA
gacaatggaatggaatggtt

SEQ ID NO: 274 - DNA, 20 bp, Homo sapiens
  source 1..20: mol_type = other DNA
acaatggaatggaatggttc

SEQ ID NO: 275 - DNA, 20 bp, Homo sapiens
  source 1..20: mol_type = other DNA
caatggaatggaatggttcg

SEQ ID NO: 276 - DNA, 20 bp, Homo sapiens
  source 1..20: mol_type = other DNA
aatggaatggaatggttcgt

SEQ ID NO: 277 - DNA, 20 bp, Homo sapiens
  source 1..20: mol_type = other DNA
atggaatggaatggttcgtg

SEQ ID NO: 278 - DNA, 20 bp, Homo sapiens
  source 1..20: mol_type = other DNA
tggaatggaatggttcgtga

SEQ ID NO: 279 - DNA, 20 bp, Homo sapiens
  source 1..20: mol_type = other DNA
ggaatggaatggttcgtgaa

SEQ ID NO: 280 - DNA, 20 bp, Homo sapiens
  source 1..20: mol_type = other DNA
gaatggaatggttcgtgaac

SEQ ID NO: 281 - DNA, 20 bp, Homo sapiens
  source 1..20: mol_type = other DNA
aatggaatggttcgtgaact

SEQ ID NO: 282 - DNA, 20 bp, Homo sapiens
  source 1..20: mol_type = other DNA
atggaatggttcgtgaacta

SEQ ID NO: 283 - DNA, 20 bp, Homo sapiens
  source 1..20: mol_type = other DNA
tggaatggttcgtgaactaa

SEQ ID NO: 284 - DNA, 20 bp, Homo sapiens
  source 1..20: mol_type = other DNA
ggaatggttcgtgaactaat

SEQ ID NO: 285 - DNA, 20 bp, Homo sapiens
  source 1..20: mol_type = other DNA
gaatggttcgtgaactaatt

SEQ ID NO: 286 - DNA, 20 bp, Homo sapiens
  source 1..20: mol_type = other DNA
aatggttcgtgaactaattg

SEQ ID NO: 287 - DNA, 20 bp, Homo sapiens
  source 1..20: mol_type = other DNA
atggttcgtgaactaattga

SEQ ID NO: 288 - DNA, 20 bp, Homo sapiens
  source 1..20: mol_type = other DNA
tggttcgtgaactaattgat

SEQ ID NO: 289 - DNA, 20 bp, Homo sapiens
  source 1..20: mol_type = other DNA
ggttcgtgaactaattgatc

SEQ ID NO: 290 - DNA, 20 bp, Homo sapiens
  source 1..20: mol_type = other DNA
gttcgtgaactaattgatca

SEQ ID NO: 291 - DNA, 20 bp, Homo sapiens
  source 1..20: mol_type = other DNA
ttcgtgaactaattgatcat

SEQ ID NO: 292 - DNA, 20 bp, Homo sapiens
  source 1..20: mol_type = other DNA
tcgtgaactaattgatcata

SEQ ID NO: 293 - DNA, 20 bp, Homo sapiens
  source 1..20: mol_type = other DNA
cgtgaactaattgatcataa

SEQ ID NO: 294 - DNA, 20 bp, Homo sapiens
  source 1..20: mol_type = other DNA
gtgaactaattgatcataaa

SEQ ID NO: 295 - DNA, 20 bp, Homo sapiens
  source 1..20: mol_type = other DNA
tgaactaattgatcataaag

SEQ ID NO: 296 - DNA, 20 bp, Homo sapiens
  source 1..20: mol_type = other DNA
gaactaattgatcataaagc

SEQ ID NO: 297 - DNA, 20 bp, Homo sapiens
  source 1..20: mol_type = other DNA
aactaattgatcataaagct

SEQ ID NO: 298 - DNA, 20 bp, Homo sapiens
  source 1..20: mol_type = other DNA
actaattgatcataaagctg

SEQ ID NO: 299 - DNA, 20 bp, Homo sapiens
  source 1..20: mol_type = other DNA
ctaattgatcataaagctga

SEQ ID NO: 300 - DNA, 20 bp, Homo sapiens
  source 1..20: mol_type = other DNA
taattgatcataaagctgac

SEQ ID NO: 301 - DNA, 20 bp, Homo sapiens
  source 1..20: mol_type = other DNA
aattgatcataaagctgacc

SEQ ID NO: 302 - DNA, 20 bp, Homo sapiens
  source 1..20: mol_type = other DNA
attgatcataaagctgacct

SEQ ID NO: 303 - DNA, 20 bp, Homo sapiens
  source 1..20: mol_type = other DNA
ttgatcataaagctgacctt

SEQ ID NO: 304 - DNA, 20 bp, Homo sapiens
  source 1..20: mol_type = other DNA
tgatcataaagctgaccttg

SEQ ID NO: 305 - DNA, 20 bp, Homo sapiens
  source 1..20: mol_type = other DNA
gggcaatggttgcccagaag

SEQ ID NO: 306 - DNA, 20 bp, Homo sapiens
  source 1..20: mol_type = other DNA
ggcaatggttgcccagaaga

SEQ ID NO: 307 - DNA, 20 bp, Homo sapiens
  source 1..20: mol_type = other DNA
gcaatggttgcccagaagag

SEQ ID NO: 308 - DNA, 20 bp, Homo sapiens
  source 1..20: mol_type = other DNA
caatggttgcccagaagagg

SEQ ID NO: 309 - DNA, 20 bp, Homo sapiens
  source 1..20: mol_type = other DNA
aatggttgcccagaagagga

SEQ ID NO: 310 - DNA, 20 bp, Homo sapiens
  source 1..20: mol_type = other DNA
ttaaaacataagccacaggc

SEQ ID NO: 311 - DNA, 20 bp, Homo sapiens
  source 1..20: mol_type = other DNA
taaaacataagccacaggcc

SEQ ID NO: 312 - DNA, 20 bp, Homo sapiens
  source 1..20: mol_type = other DNA
aaaacataagccacaggccc

SEQ ID NO: 313 - DNA, 20 bp, Homo sapiens
  source 1..20: mol_type = other DNA
aaacataagccacaggcccc

SEQ ID NO: 314 - DNA, 20 bp, Homo sapiens
  source 1..20: mol_type = other DNA
aacataagccacaggcccca

SEQ ID NO: 315 - DNA, 20 bp, Homo sapiens
  source 1..20: mol_type = other DNA
acataagccacaggccccag

SEQ ID NO: 316 - DNA, 20 bp, Homo sapiens
  source 1..20: mol_type = other DNA
cataagccacaggccccagt

SEQ ID NO: 317 - DNA, 20 bp, Homo sapiens
  source 1..20: mol_type = other DNA
ataagccacaggccccagtt

SEQ ID NO: 318 - DNA, 20 bp, Homo sapiens
  source 1..20: mol_type = other DNA
taagccacaggccccagtta

SEQ ID NO: 319 - DNA, 20 bp, Homo sapiens
  source 1..20: mol_type = other DNA
aagccacaggccccagttat

SEQ ID NO: 320 - DNA, 20 bp, Homo sapiens
  source 1..20: mol_type = other DNA
agccacaggccccagttatt

SEQ ID NO: 321 - DNA, 20 bp, Homo sapiens
  source 1..20: mol_type = other DNA
gccacaggccccagttattg

SEQ ID NO: 322 - DNA, 20 bp, Homo sapiens
  source 1..20: mol_type = other DNA
ccacaggccccagttattgt

SEQ ID NO: 323 - DNA, 20 bp, Homo sapiens
  source 1..20: mol_type = other DNA
cacaggccccagttattgtg

SEQ ID NO: 324 - DNA, 20 bp, Homo sapiens
  source 1..20: mol_type = other DNA
acaggccccagttattgtga

SEQ ID NO: 325 - DNA, 20 bp, Homo sapiens
  source 1..20: mol_type = other DNA
caggccccagttattgtgaa

SEQ ID NO: 326 - DNA, 20 bp, Homo sapiens
  source 1..20: mol_type = other DNA
aggccccagttattgtgaaa

SEQ ID NO: 327 - DNA, 20 bp, Homo sapiens
  source 1..20: mol_type = other DNA
ggccccagttattgtgaaaa

SEQ ID NO: 328 - DNA, 20 bp, Homo sapiens
  source 1..20: mol_type = other DNA
gccccagttattgtgaaaac

SEQ ID NO: 329 - DNA, 20 bp, Homo sapiens
  source 1..20: mol_type = other DNA
ccccagttattgtgaaaaca

SEQ ID NO: 330 - DNA, 20 bp, Homo sapiens
  source 1..20: mol_type = other DNA
cccagttattgtgaaaacag

SEQ ID NO: 331 - DNA, 20 bp, Homo sapiens
  source 1..20: mol_type = other DNA
aaacaatttagccagaatgt

SEQ ID NO: 332 - DNA, 20 bp, Homo sapiens
  source 1..20: mol_type = other DNA
aacaatttagccagaatgtt

SEQ ID NO: 333 - DNA, 20 bp, Homo sapiens
  source 1..20: mol_type = other DNA
acaatttagccagaatgttt

SEQ ID NO: 334 - DNA, 20 bp, Homo sapiens
  source 1..20: mol_type = other DNA
caatttagccagaatgtttc

SEQ ID NO: 335 - DNA, 20 bp, Homo sapiens
  source 1..20: mol_type = other DNA
aatttagccagaatgtttcc

SEQ ID NO: 336 - DNA, 20 bp, Homo sapiens
  source 1..20: mol_type = other DNA
atttagccagaatgtttcct

SEQ ID NO: 337 - DNA, 20 bp, Homo sapiens
  source 1..20: mol_type = other DNA
tttagccagaatgtttcctg

SEQ ID NO: 338 - DNA, 20 bp, Homo sapiens
  source 1..20: mol_type = other DNA
ttagccagaatgtttcctgt

SEQ ID NO: 339 - DNA, 20 bp, Homo sapiens
  source 1..20: mol_type = other DNA
gtctatgaactagagactct

SEQ ID NO: 340 - DNA, 20 bp, Homo sapiens
  source 1..20: mol_type = other DNA
tctatgaactagagactctg

SEQ ID NO: 341 - DNA, 20 bp, Homo sapiens
  source 1..20: mol_type = other DNA
ctatgaactagagactctgt

SEQ ID NO: 342 - DNA, 20 bp, Homo sapiens
  source 1..20: mol_type = other DNA
tatgaactagagactctgtg

SEQ ID NO: 343 - DNA, 20 bp, Homo sapiens
  source 1..20: mol_type = other DNA
aaccaataaaagtgtcacta

SEQ ID NO: 344 - DNA, 20 bp, Homo sapiens
  source 1..20: mol_type = other DNA
accaataaaagtgtcactaa

SEQ ID NO: 345 - DNA, 20 bp, Homo sapiens
  source 1..20: mol_type = other DNA
ccaataaaagtgtcactaag

SEQ ID NO: 346 - DNA, 20 bp, Homo sapiens
  source 1..20: mol_type = other DNA
caataaaagtgtcactaaga

SEQ ID NO: 347 - DNA, 20 bp, Homo sapiens
  source 1..20: mol_type = other DNA
aataaaagtgtcactaagaa

SEQ ID NO: 348 - DNA, 20 bp, Homo sapiens
  source 1..20: mol_type = other DNA
ataaaagtgtcactaagaat

SEQ ID NO: 349 - DNA, 20 bp, Homo sapiens
  source 1..20: mol_type = other DNA
taaaagtgtcactaagaata

SEQ ID NO: 350 - DNA, 20 bp, Homo sapiens
  source 1..20: mol_type = other DNA
aaaagtgtcactaagaatat

SEQ ID NO: 351 - DNA, 20 bp, Homo sapiens
  source 1..20: mol_type = other DNA
aaagtgtcactaagaatata

SEQ ID NO: 352 - DNA, 20 bp, Homo sapiens
  source 1..20: mol_type = other DNA
aagtgtcactaagaatataa

SEQ ID NO: 353 - DNA, 20 bp, Homo sapiens
  source 1..20: mol_type = other DNA
agtgtcactaagaatataaa

SEQ ID NO: 354 - DNA, 20 bp, Homo sapiens
  source 1..20: mol_type = other DNA
gtgtcactaagaatataaat

SEQ ID NO: 355 - DNA, 20 bp, Homo sapiens
  source 1..20: mol_type = other DNA
tgtcactaagaatataaata

SEQ ID NO: 356 - DNA, 20 bp, Homo sapiens
  source 1..20: mol_type = other DNA
ctaacccccatatcccaaat

SEQ ID NO: 357 - DNA, 20 bp, Homo sapiens
  source 1..20: mol_type = other DNA
taacccccatatcccaaata

SEQ ID NO: 358 - DNA, 20 bp, Homo sapiens
  source 1..20: mol_type = other DNA
aacccccatatcccaaataa

SEQ ID NO: 359 - DNA, 20 bp, Homo sapiens
  source 1..20: mol_type = other DNA
acccccatatcccaaataat

SEQ ID NO: 360 - DNA, 20 bp, Homo sapiens
  source 1..20: mol_type = other DNA
cccccatatcccaaataata

SEQ ID NO: 361 - DNA, 20 bp, Homo sapiens
  source 1..20: mol_type = other DNA
ccccatatcccaaataataa

SEQ ID NO: 362 - DNA, 20 bp, Homo sapiens
  source 1..20: mol_type = other DNA
cccatatcccaaataataat

SEQ ID NO: 363 - DNA, 20 bp, Homo sapiens
  source 1..20: mol_type = other DNA
ccatatcccaaataataata

SEQ ID NO: 364 - DNA, 20 bp, Homo sapiens
  source 1..20: mol_type = other DNA
catatcccaaataataataa

SEQ ID NO: 365 - DNA, 20 bp, Homo sapiens
  source 1..20: mol_type = other DNA
atatcccaaataataataat

SEQ ID NO: 366 - DNA, 20 bp, Homo sapiens
  source 1..20: mol_type = other DNA
tatcccaaataataataata

SEQ ID NO: 367 - DNA, 20 bp, Homo sapiens
  source 1..20: mol_type = other DNA
atcccaaataataataataa

SEQ ID NO: 368 - DNA, 20 bp, Homo sapiens
  source 1..20: mol_type = other DNA
tcccaaataataataataat

SEQ ID NO: 369 - DNA, 20 bp, Homo sapiens
  source 1..20: mol_type = other DNA
cccaaataataataataata

SEQ ID NO: 370 - DNA, 20 bp, Homo sapiens
  source 1..20: mol_type = other DNA
ccaaataataataataataa

SEQ ID NO: 371 - DNA, 20 bp, Homo sapiens
  source 1..20: mol_type = other DNA
caaataataataataataat

SEQ ID NO: 372 - DNA, 20 bp, Homo sapiens
  source 1..20: mol_type = other DNA
aaataataataataataata

SEQ ID NO: 373 - DNA, 20 bp, Homo sapiens
  source 1..20: mol_type = other DNA
aataataataataataataa

SEQ ID NO: 374 - DNA, 20 bp, Homo sapiens
  source 1..20: mol_type = other DNA
ataataataataataataat

SEQ ID NO: 375 - DNA, 20 bp, Homo sapiens
  source 1..20: mol_type = other DNA
taataataataataataata

SEQ ID NO: 376 - DNA, 20 bp, Homo sapiens
  source 1..20: mol_type = other DNA
aataataataataataataa

SEQ ID NO: 377 - DNA, 20 bp, Homo sapiens
  source 1..20: mol_type = other DNA
ataataataataataataat

SEQ ID NO: 378 - DNA, 20 bp, Homo sapiens
  source 1..20: mol_type = other DNA
taataataataataataata

SEQ ID NO: 379 - DNA, 20 bp, Homo sapiens
  source 1..20: mol_type = other DNA
aataataataataataataa

SEQ ID NO: 380 - DNA, 20 bp, Homo sapiens
  source 1..20: mol_type = other DNA
ataataataataataataat

SEQ ID NO: 381 - DNA, 20 bp, Homo sapiens
  source 1..20: mol_type = other DNA
taataataataataataata

SEQ ID NO: 382 - DNA, 20 bp, Homo sapiens
  source 1..20: mol_type = other DNA
aataataataataataataa

SEQ ID NO: 383 - DNA, 20 bp, Homo sapiens
  source 1..20: mol_type = other DNA
ataataataataataataat

SEQ ID NO: 384 - DNA, 20 bp, Homo sapiens
  source 1..20: mol_type = other DNA
taataataataataataata

SEQ ID NO: 385 - DNA, 20 bp, Homo sapiens
  source 1..20: mol_type = other DNA
aataataataataataataa

SEQ ID NO: 386 - DNA, 20 bp, Homo sapiens
  source 1..20: mol_type = other DNA
ataataataataataataat

SEQ ID NO: 387 - DNA, 20 bp, Homo sapiens
  source 1..20: mol_type = other DNA
taataataataataataata

SEQ ID NO: 388 - DNA, 20 bp, Homo sapiens
  source 1..20: mol_type = other DNA
aataataataataataataa

SEQ ID NO: 389 - DNA, 20 bp, Homo sapiens
  source 1..20: mol_type = other DNA
ataataataataataataat

SEQ ID NO: 390 - DNA, 20 bp, Homo sapiens
  source 1..20: mol_type = other DNA
taataataataataataata

SEQ ID NO: 391 - DNA, 20 bp, Homo sapiens
  source 1..20: mol_type = other DNA
aataataataataataataa

SEQ ID NO: 392 - DNA, 20 bp, Homo sapiens
  source 1..20: mol_type = other DNA
ataataataataataataat

SEQ ID NO: 393 - DNA, 20 bp, Homo sapiens
  source 1..20: mol_type = other DNA
taataataataataataata

SEQ ID NO: 394 - DNA, 20 bp, Homo sapiens
  source 1..20: mol_type = other DNA
aataataataataataataa

SEQ ID NO: 395 - DNA, 20 bp, Homo sapiens
  source 1..20: mol_type = other DNA
ataataataataataataat

SEQ ID NO: 396 - DNA, 20 bp, Homo sapiens
  source 1..20: mol_type = other DNA
taataataataataataata

SEQ ID NO: 397 - DNA, 20 bp, Homo sapiens
  source 1..20: mol_type = other DNA
aataataataataataataa

SEQ ID NO: 398 - DNA, 20 bp, Homo sapiens
  source 1..20: mol_type = other DNA
ataataataataataataaa

SEQ ID NO: 399 - DNA, 20 bp, Homo sapiens
  source 1..20: mol_type = other DNA
taataataataataataaaa

SEQ ID NO: 400 - DNA, 20 bp, Homo sapiens
  source 1..20: mol_type = other DNA
aataataataataataaaag

SEQ ID NO: 401 - DNA, 20 bp, Homo sapiens
  source 1..20: mol_type = other DNA
ataataataataataaaagc

SEQ ID NO: 402 - DNA, 20 bp, Homo sapiens
  source 1..20: mol_type = other DNA
taataataataataaaagca

SEQ ID NO: 403 - DNA, 20 bp, Homo sapiens
  source 1..20: mol_type = other DNA
aataataataataaaagcag

SEQ ID NO: 404 - DNA, 20 bp, Homo sapiens
  source 1..20: mol_type = other DNA
ataataataataaaagcagt

SEQ ID NO: 405 - DNA, 20 bp, Homo sapiens
  source 1..20: mol_type = other DNA
taataataataaaagcagtt

SEQ ID NO: 406 - DNA, 20 bp, Homo sapiens
  source 1..20: mol_type = other DNA
aaaattaaaaaaaatcatta

SEQ ID NO: 407 - DNA, 20 bp, Homo sapiens
  source 1..20: mol_type = other DNA
aaattaaaaaaaatcattaa

SEQ ID NO: 408 - DNA, 20 bp, Homo sapiens
  source 1..20: mol_type = other DNA
aattaaaaaaaatcattaaa

SEQ ID NO: 409 - DNA, 20 bp, Homo sapiens
  source 1..20: mol_type = other DNA
attaaaaaaaatcattaaaa

SEQ ID NO: 410 - DNA, 20 bp, Homo sapiens
  source 1..20: mol_type = other DNA
ttaaaaaaaatcattaaaac

SEQ ID NO: 411 - DNA, 20 bp, Homo sapiens
  source 1..20: mol_type = other DNA
taaaaaaaatcattaaaact

SEQ ID NO: 412 - DNA, 20 bp, Homo sapiens
  source 1..20: mol_type = other DNA
aaaaaaaatcattaaaacta

SEQ ID NO: 413 - DNA, 20 bp, Homo sapiens
  source 1..20: mol_type = other DNA
aaaaaaatcattaaaactat

SEQ ID NO: 414 - DNA, 20 bp, Homo sapiens
  source 1..20: mol_type = other DNA
aaaaaatcattaaaactatg

SEQ ID NO: 415 - DNA, 20 bp, Homo sapiens
  source 1..20: mol_type = other DNA
aaaaatcattaaaactatgt

SEQ ID NO: 416 - DNA, 20 bp, Homo sapiens
  source 1..20: mol_type = other DNA
aaaatcattaaaactatgtt

SEQ ID NO: 417 - DNA, 20 bp, Homo sapiens
  source 1..20: mol_type = other DNA
aaatcattaaaactatgttc

SEQ ID NO: 418 - DNA, 20 bp, Homo sapiens
  source 1..20: mol_type = other DNA
aatcattaaaactatgttca

SEQ ID NO: 419 - DNA, 20 bp, Homo sapiens
  source 1..20: mol_type = other DNA
atcattaaaactatgttcat

SEQ ID NO: 420 - DNA, 20 bp, Homo sapiens
  source 1..20: mol_type = other DNA
tcattaaaactatgttcatt

SEQ ID NO: 421 - DNA, 20 bp, Homo sapiens
  source 1..20: mol_type = other DNA
cattaaaactatgttcattt

SEQ ID NO: 422 - DNA, 20 bp, Homo sapiens
  source 1..20: mol_type = other DNA
attaaaactatgttcatttt

SEQ ID NO: 423 - DNA, 20 bp, Homo sapiens
  source 1..20: mol_type = other DNA
ttaaaactatgttcatttta

SEQ ID NO: 424 - DNA, 20 bp, Homo sapiens
  source 1..20: mol_type = other DNA
taaaactatgttcattttac

SEQ ID NO: 425 - DNA, 20 bp, Homo sapiens
  source 1..20: mol_type = other DNA
aaaactatgttcattttact

SEQ ID NO: 426 - DNA, 20 bp, Homo sapiens
  source 1..20: mol_type = other DNA
aaactatgttcattttactt

SEQ ID NO: 427 - DNA, 20 bp, Homo sapiens
  source 1..20: mol_type = other DNA
aactatgttcattttacttt

SEQ ID NO: 428 - DNA, 20 bp, Homo sapiens
  source 1..20: mol_type = other DNA
actatgttcattttactttc

SEQ ID NO: 429 - DNA, 20 bp, Homo sapiens
  source 1..20: mol_type = other DNA
ctatgttcattttactttca

SEQ ID NO: 430 - DNA, 20 bp, Homo sapiens
  source 1..20: mol_type = other DNA
caagttagataatataaaag

SEQ ID NO: 431 - DNA, 20 bp, Homo sapiens
  source 1..20: mol_type = other DNA
aagttagataatataaaagc

SEQ ID NO: 432 - DNA, 20 bp, Homo sapiens
  source 1..20: mol_type = other DNA
agttagataatataaaagct

SEQ ID NO: 433 - DNA, 20 bp, Homo sapiens
  source 1..20: mol_type = other DNA
gttagataatataaaagcta

SEQ ID NO: 434 - DNA, 20 bp, Homo sapiens
  source 1..20: mol_type = other DNA
ttagataatataaaagctag

SEQ ID NO: 435 - DNA, 20 bp, Homo sapiens
  source 1..20: mol_type = other DNA
tttggtggtttttcacactt

SEQ ID NO: 436 - DNA, 20 bp, Homo sapiens
  source 1..20: mol_type = other DNA
ttggtggtttttcacactta

SEQ ID NO: 437 - DNA, 20 bp, Homo sapiens
  source 1..20: mol_type = other DNA
tggtggtttttcacacttat

SEQ ID NO: 438 - DNA, 20 bp, Homo sapiens
  source 1..20: mol_type = other DNA
ggtggtttttcacacttatc

SEQ ID NO: 439 - DNA, 20 bp, Homo sapiens
  source 1..20: mol_type = other DNA
gtggtttttcacacttatca

SEQ ID NO: 440 - DNA, 20 bp, Homo sapiens
  source 1..20: mol_type = other DNA
tggtttttcacacttatcat

SEQ ID NO: 441 - DNA, 20 bp, Homo sapiens
  source 1..20: mol_type = other DNA
ggtttttcacacttatcatc

SEQ ID NO: 442 - DNA, 20 bp, Homo sapiens
  source 1..20: mol_type = other DNA
gtttttcacacttatcatca

SEQ ID NO: 443 - DNA, 20 bp, Homo sapiens
  source 1..20: mol_type = other DNA
tttttcacacttatcatcat

SEQ ID NO: 444 - DNA, 20 bp, Homo sapiens
  source 1..20: mol_type = other DNA
ttttcacacttatcatcatt

SEQ ID NO: 445 - DNA, 20 bp, Homo sapiens
  source 1..20: mol_type = other DNA
tttcacacttatcatcattt

SEQ ID NO: 446 - DNA, 20 bp, Homo sapiens
  source 1..20: mol_type = other DNA
ttcacacttatcatcatttc

SEQ ID NO: 447 - DNA, 20 bp, Homo sapiens
  source 1..20: mol_type = other DNA
tcacacttatcatcatttct

SEQ ID NO: 448 - DNA, 20 bp, Homo sapiens
  source 1..20: mol_type = other DNA
cacacttatcatcatttctt

SEQ ID NO: 449 - DNA, 20 bp, Homo sapiens
  source 1..20: mol_type = other DNA
acacttatcatcatttcttc

SEQ ID NO: 450 - DNA, 20 bp, Homo sapiens
  source 1..20: mol_type = other DNA
cacttatcatcatttcttcg

SEQ ID NO: 451 - DNA, 20 bp, Homo sapiens
  source 1..20: mol_type = other DNA
acttatcatcatttcttcgt

SEQ ID NO: 452 - DNA, 20 bp, Homo sapiens
  source 1..20: mol_type = other DNA
cttatcatcatttcttcgta

SEQ ID NO: 453 - DNA, 20 bp, Homo sapiens
  source 1..20: mol_type = other DNA
ttatcatcatttcttcgtat

SEQ ID NO: 454 - DNA, 20 bp, Homo sapiens
  source 1..20: mol_type = other DNA
tatcatcatttcttcgtata

SEQ ID NO: 455 - DNA, 20 bp, Homo sapiens
  source 1..20: mol_type = other DNA
atcatcatttcttcgtatac

SEQ ID NO: 456 - DNA, 20 bp, Homo sapiens
  source 1..20: mol_type = other DNA
tcatcatttcttcgtatact

SEQ ID NO: 457 - DNA, 20 bp, Homo sapiens
  source 1..20: mol_type = other DNA
catcatttcttcgtatactg

SEQ ID NO: 458 - DNA, 20 bp, Homo sapiens
  source 1..20: mol_type = other DNA
atcatttcttcgtatactgc

SEQ ID NO: 459 - DNA, 20 bp, Homo sapiens
  source 1..20: mol_type = other DNA
tcatttcttcgtatactgct

SEQ ID NO: 460 - DNA, 20 bp, Homo sapiens
  source 1..20: mol_type = other DNA
catttcttcgtatactgcta

SEQ ID NO: 461 - DNA, 20 bp, Homo sapiens
  source 1..20: mol_type = other DNA
atttcttcgtatactgctaa

SEQ ID NO: 462 - DNA, 20 bp, Homo sapiens
  source 1..20: mol_type = other DNA
tttcttcgtatactgctaac

SEQ ID NO: 463 - DNA, 20 bp, Homo sapiens
  source 1..20: mol_type = other DNA
ttcttcgtatactgctaact

SEQ ID NO: 464 - DNA, 20 bp, Homo sapiens
  source 1..20: mol_type = other DNA
tcttcgtatactgctaactt

SEQ ID NO: 465 - DNA, 20 bp, Homo sapiens
  source 1..20: mol_type = other DNA
cttcgtatactgctaactta

SEQ ID NO: 466 - DNA, 20 bp, Homo sapiens
  source 1..20: mol_type = other DNA
ttcgtatactgctaacttag

SEQ ID NO: 467 - DNA, 20 bp, Homo sapiens
  source 1..20: mol_type = other DNA
tcgtatactgctaacttagc

SEQ ID NO: 468 - DNA, 20 bp, Homo sapiens
  source 1..20: mol_type = other DNA
cgtatactgctaacttagcc

SEQ ID NO: 469 - DNA, 20 bp, Homo sapiens
  source 1..20: mol_type = other DNA
gtatactgctaacttagccg

SEQ ID NO: 470 - DNA, 20 bp, Homo sapiens
  source 1..20: mol_type = other DNA
tatactgctaacttagccgc

SEQ ID NO: 471 - DNA, 20 bp, Homo sapiens
  source 1..20: mol_type = other DNA
atactgctaacttagccgcc

SEQ ID NO: 472 - DNA, 20 bp, Homo sapiens
  source 1..20: mol_type = other DNA
tactgctaacttagccgcct

SEQ ID NO: 473 - DNA, 20 bp, Homo sapiens
  source 1..20: mol_type = other DNA
actgctaacttagccgcctt

SEQ ID NO: 474 - DNA, 20 bp, Homo sapiens
  source 1..20: mol_type = other DNA
ctgctaacttagccgccttt

SEQ ID NO: 475 - DNA, 20 bp, Homo sapiens
  source 1..20: mol_type = other DNA
tgctaacttagccgcctttc

SEQ ID NO: 476 - DNA, 20 bp, Homo sapiens
  source 1..20: mol_type = other DNA
gctaacttagccgcctttct

SEQ ID NO: 477 - DNA, 20 bp, Homo sapiens
  source 1..20: mol_type = other DNA
ctaacttagccgcctttctg

SEQ ID NO: 478 - DNA, 20 bp, Homo sapiens
  source 1..20: mol_type = other DNA
taacttagccgcctttctga

SEQ ID NO: 479 - DNA, 20 bp, Homo sapiens
  source 1..20: mol_type = other DNA
aacttagccgcctttctgac

SEQ ID NO: 480 - DNA, 20 bp, Homo sapiens
  source 1..20: mol_type = other DNA
acttagccgcctttctgaca

SEQ ID NO: 481 - DNA, 20 bp, Homo sapiens
  source 1..20: mol_type = other DNA
cttagccgcctttctgacag

SEQ ID NO: 482 - DNA, 20 bp, Homo sapiens
  source 1..20: mol_type = other DNA
ttagccgcctttctgacagt

SEQ ID NO: 483 - DNA, 20 bp, Homo sapiens
  source 1..20: mol_type = other DNA
tagccgcctttctgacagtg

SEQ ID NO: 484 - DNA, 20 bp, Homo sapiens
  source 1..20: mol_type = other DNA
agccgcctttctgacagtgg

SEQ ID NO: 485 - DNA, 20 bp, Homo sapiens
  source 1..20: mol_type = other DNA
gccgcctttctgacagtgga

SEQ ID NO: 486 - DNA, 20 bp, Homo sapiens
  source 1..20: mol_type = other DNA
ccgcctttctgacagtggaa

SEQ ID NO: 487 - DNA, 20 bp, Homo sapiens
  source 1..20: mol_type = other DNA
cgcctttctgacagtggaac

SEQ ID NO: 488 - DNA, 20 bp, Homo sapiens
  source 1..20: mol_type = other DNA
gcctttctgacagtggaacg

SEQ ID NO: 489 - DNA, 20 bp, Homo sapiens
  source 1..20: mol_type = other DNA
cctttctgacagtggaacgc

SEQ ID NO: 490 - DNA, 20 bp, Homo sapiens
  source 1..20: mol_type = other DNA
ctttctgacagtggaacgca

SEQ ID NO: 491 - DNA, 20 bp, Homo sapiens
  source 1..20: mol_type = other DNA
tttctgacagtggaacgcat

SEQ ID NO: 492 - DNA, 20 bp, Homo sapiens
  source 1..20: mol_type = other DNA
ttctgacagtggaacgcatg

SEQ ID NO: 493 - DNA, 20 bp, Homo sapiens
  source 1..20: mol_type = other DNA
tctgacagtggaacgcatgg

SEQ ID NO: 494 - DNA, 20 bp, Homo sapiens
  source 1..20: mol_type = other DNA
ctgacagtggaacgcatgga

SEQ ID NO: 495 - DNA, 20 bp, Homo sapiens
  source 1..20: mol_type = other DNA
ggtgcaaccatgactttttt

SEQ ID NO: 496 - DNA, 20 bp, Homo sapiens
  source 1..20: mol_type = other DNA
gtgcaaccatgacttttttc

SEQ ID NO: 497 - DNA, 20 bp, Homo sapiens
  source 1..20: mol_type = other DNA
tgcaaccatgacttttttca

SEQ ID NO: 498 - DNA, 20 bp, Homo sapiens
  source 1..20: mol_type = other DNA
gcaaccatgacttttttcaa

SEQ ID NO: 499 - DNA, 20 bp, Homo sapiens
  source 1..20: mol_type = other DNA
caaccatgacttttttcaag

SEQ ID NO: 500 - DNA, 20 bp, Homo sapiens
  source 1..20: mol_type = other DNA
aaccatgacttttttcaaga

SEQ ID NO: 501 - DNA, 20 bp, Homo sapiens
  source 1..20: mol_type = other DNA
accatgacttttttcaagaa

SEQ ID NO: 502 - DNA, 20 bp, Homo sapiens
  source 1..20: mol_type = other DNA
ccatgacttttttcaagaaa

SEQ ID NO: 503 - DNA, 20 bp, Homo sapiens
  source 1..20: mol_type = other DNA
catgacttttttcaagaaat

SEQ ID NO: 504 - DNA, 20 bp, Homo sapiens
  source 1..20: mol_type = other DNA
atgacttttttcaagaaatc

SEQ ID NO: 505 - DNA, 20 bp, Homo sapiens
  source 1..20: mol_type = other DNA
tgacttttttcaagaaatca

SEQ ID NO: 506 - DNA, 20 bp, Homo sapiens
  source 1..20: mol_type = other DNA
gacttttttcaagaaatcaa

SEQ ID NO: 507 - DNA, 20 bp, Homo sapiens
  source 1..20: mol_type = other DNA
acttttttcaagaaatcaaa

SEQ ID NO: 508 - DNA, 20 bp, Homo sapiens
  source 1..20: mol_type = other DNA
cttttttcaagaaatcaaaa

SEQ ID NO: 509 - DNA, 20 bp, Homo sapiens
  source 1..20: mol_type = other DNA
ttttttcaagaaatcaaaaa

SEQ ID NO: 510 - DNA, 20 bp, Homo sapiens
  source 1..20: mol_type = other DNA
tttttcaagaaatcaaaaat

SEQ ID NO: 511 - DNA, 20 bp, Homo sapiens
  source 1..20: mol_type = other DNA
ttttcaagaaatcaaaaatc

SEQ ID NO: 512 - DNA, 20 bp, Homo sapiens
  source 1..20: mol_type = other DNA
tttcaagaaatcaaaaatct

SEQ ID NO: 513 - DNA, 20 bp, Homo sapiens
  source 1..20: mol_type = other DNA
aggttccttttgaagctcaa

SEQ ID NO: 514 - DNA, 20 bp, Homo sapiens
  source 1..20: mol_type = other DNA
ggttccttttgaagctcaac

SEQ ID NO: 515 - DNA, 20 bp, Homo sapiens
  source 1..20: mol_type = other DNA
gttccttttgaagctcaact

SEQ ID NO: 516 - DNA, 20 bp, Homo sapiens
  source 1..20: mol_type = other DNA
ttccttttgaagctcaactg

SEQ ID NO: 517 - DNA, 20 bp, Homo sapiens
  source 1..20: mol_type = other DNA
tccttttgaagctcaactgt

SEQ ID NO: 518 - DNA, 20 bp, Homo sapiens
  source 1..20: mol_type = other DNA
ccttttgaagctcaactgtt

SEQ ID NO: 519 - DNA, 20 bp, Homo sapiens
  source 1..20: mol_type = other DNA
cttttgaagctcaactgttg

SEQ ID NO: 520 - DNA, 20 bp, Homo sapiens
  source 1..20: mol_type = other DNA
ttttgaagctcaactgttgc

SEQ ID NO: 521 - DNA, 20 bp, Homo sapiens
  source 1..20: mol_type = other DNA
tttgaagctcaactgttgcc

SEQ ID NO: 522 - DNA, 20 bp, Homo sapiens
  source 1..20: mol_type = other DNA
ttgaagctcaactgttgcca

SEQ ID NO: 523 - DNA, 20 bp, Homo sapiens
  source 1..20: mol_type = other DNA
tgaagctcaactgttgccag

SEQ ID NO: 524 - DNA, 20 bp, Homo sapiens
  source 1..20: mol_type = other DNA
gaagctcaactgttgccagg

SEQ ID NO: 525 - DNA, 20 bp, Homo sapiens
  source 1..20: mol_type = other DNA
aagctcaactgttgccagga

SEQ ID NO: 526 - DNA, 20 bp, Homo sapiens
  source 1..20: mol_type = other DNA
agctcaactgttgccaggag

SEQ ID NO: 527 - DNA, 20 bp, Homo sapiens
  source 1..20: mol_type = other DNA
gctcaactgttgccaggaga

SEQ ID NO: 528 - DNA, 20 bp, Homo sapiens
  source 1..20: mol_type = other DNA
ctcaactgttgccaggagat

SEQ ID NO: 529 - DNA, 20 bp, Homo sapiens
  source 1..20: mol_type = other DNA
tcaactgttgccaggagatg

SEQ ID NO: 530 - DNA, 20 bp, Homo sapiens
  source 1..20: mol_type = other DNA
caactgttgccaggagatgg

SEQ ID NO: 531 - DNA, 20 bp, Homo sapiens
  source 1..20: mol_type = other DNA
aactgttgccaggagatgga

SEQ ID NO: 532 - DNA, 20 bp, Homo sapiens
  source 1..20: mol_type = other DNA
actgttgccaggagatggaa

SEQ ID NO: 533 - DNA, 20 bp, Homo sapiens
  source 1..20: mol_type = other DNA
ctgttgccaggagatggaat

SEQ ID NO: 534 - DNA, 20 bp, Homo sapiens
  source 1..20: mol_type = other DNA
tgttgccaggagatggaata

SEQ ID NO: 535 - DNA, 20 bp, Homo sapiens
  source 1..20: mol_type = other DNA
gttgccaggagatggaatat

SEQ ID NO: 536 - DNA, 20 bp, Homo sapiens
  source 1..20: mol_type = other DNA
ttgccaggagatggaatatc

SEQ ID NO: 537 - DNA, 20 bp, Homo sapiens
  source 1..20: mol_type = other DNA
tgccaggagatggaatatca

SEQ ID NO: 538 - DNA, 20 bp, Homo sapiens
  source 1..20: mol_type = other DNA
gccaggagatggaatatcaa

SEQ ID NO: 539 - DNA, 20 bp, Homo sapiens
  source 1..20: mol_type = other DNA
catggcagttggaatataaa

SEQ ID NO: 540 - DNA, 20 bp, Homo sapiens
  source 1..20: mol_type = other DNA
atggcagttggaatataaag

SEQ ID NO: 541 - DNA, 20 bp, Homo sapiens
  source 1..20: mol_type = other DNA
tggcagttggaatataaagc

SEQ ID NO: 542 - DNA, 20 bp, Homo sapiens
  source 1..20: mol_type = other DNA
ggcagttggaatataaagca

SEQ ID NO: 543 - DNA, 20 bp, Homo sapiens
  source 1..20: mol_type = other DNA
gcagttggaatataaagcag

SEQ ID NO: 544 - DNA, 20 bp, Homo sapiens
  source 1..20: mol_type = other DNA
cagttggaatataaagcaga

SEQ ID NO: 545 - DNA, 20 bp, Homo sapiens
  source 1..20: mol_type = other DNA
agttggaatataaagcagat

SEQ ID NO: 546 - DNA, 20 bp, Homo sapiens
  source 1..20: mol_type = other DNA
gttggaatataaagcagatg

SEQ ID NO: 547 - DNA, 20 bp, Homo sapiens
  source 1..20: mol_type = other DNA
ttggaatataaagcagatgt

SEQ ID NO: 548 - DNA, 20 bp, Homo sapiens
  source 1..20: mol_type = other DNA
tggaatataaagcagatgtt

SEQ ID NO: 549 - DNA, 20 bp, Homo sapiens
  source 1..20: mol_type = other DNA
ggaatataaagcagatgttc

SEQ ID NO: 550 - DNA, 20 bp, Homo sapiens
  source 1..20: mol_type = other DNA
gaatataaagcagatgttca

SEQ ID NO: 551 - DNA, 20 bp, Homo sapiens
  source 1..20: mol_type = other DNA
aatataaagcagatgttcat

SEQ ID NO: 552 - DNA, 20 bp, Homo sapiens
  source 1..20: mol_type = other DNA
atataaagcagatgttcatc

SEQ ID NO: 553 - DNA, 20 bp, Homo sapiens
  source 1..20: mol_type = other DNA
tataaagcagatgttcatca

SEQ ID NO: 554 - DNA, 20 bp, Homo sapiens
  source 1..20: mol_type = other DNA
ataaagcagatgttcatcac

SEQ ID NO: 555 - DNA, 20 bp, Homo sapiens
  source 1..20: mol_type = other DNA
taaagcagatgttcatcact

SEQ ID NO: 556 - DNA, 20 bp, Homo sapiens
  source 1..20: mol_type = other DNA
aaagcagatgttcatcactt

SEQ ID NO: 557 - DNA, 20 bp, Homo sapiens
  source 1..20: mol_type = other DNA
aagcagatgttcatcactta

SEQ ID NO: 558 - DNA, 20 bp, Homo sapiens
  source 1..20: mol_type = other DNA
agcagatgttcatcacttat

SEQ ID NO: 559 - DNA, 20 bp, Homo sapiens
  source 1..20: mol_type = other DNA
gcagatgttcatcacttatt

SEQ ID NO: 560 - DNA, 20 bp, Homo sapiens
  source 1..20: mol_type = other DNA
cagatgttcatcacttattt

SEQ ID NO: 561 - DNA, 20 bp, Homo sapiens
  source 1..20: mol_type = other DNA
agatgttcatcacttatttt

SEQ ID NO: 562 - DNA, 20 bp, Homo sapiens
  source 1..20: mol_type = other DNA
gatgttcatcacttattttc

SEQ ID NO: 563 - DNA, 20 bp, Homo sapiens
  source 1..20: mol_type = other DNA
atgttcatcacttattttcc

SEQ ID NO: 564 - DNA, 20 bp, Homo sapiens
  source 1..20: mol_type = other DNA
tgttcatcacttattttcct

SEQ ID NO: 565 - DNA, 20 bp, Homo sapiens
  source 1..20: mol_type = other DNA
gttcatcacttattttcctt

SEQ ID NO: 566 - DNA, 20 bp, Homo sapiens
  source 1..20: mol_type = other DNA
ttcatcacttattttccttt

SEQ ID NO: 567 - DNA, 20 bp, Homo sapiens
  source 1..20: mol_type = other DNA
tcatcacttattttcctttt

SEQ ID NO: 568 - DNA, 20 bp, Homo sapiens
  source 1..20: mol_type = other DNA
catcacttattttccttttt

SEQ ID NO: 569 - DNA, 20 bp, Homo sapiens
  source 1..20: mol_type = other DNA
atcacttattttcctttttt

SEQ ID NO: 570 - DNA, 20 bp, Homo sapiens
  source 1..20: mol_type = other DNA
tcacttattttccttttttc

SEQ ID NO: 571 - DNA, 20 bp, Homo sapiens
  source 1..20: mol_type = other DNA
cacttattttccttttttct

SEQ ID NO: 572 - DNA, 20 bp, Homo sapiens
  source 1..20: mol_type = other DNA
acttattttccttttttctt

SEQ ID NO: 573 - DNA, 20 bp, Homo sapiens
  source 1..20: mol_type = other DNA
atgccaattatcaaggaaat

SEQ ID NO: 574 - DNA, 20 bp, Homo sapiens
  source 1..20: mol_type = other DNA
tgccaattatcaaggaaata

SEQ ID NO: 575 - DNA, 20 bp, Homo sapiens
  source 1..20: mol_type = other DNA
gccaattatcaaggaaataa

SEQ ID NO: 576 - DNA, 20 bp, Homo sapiens
  source 1..20: mol_type = other DNA
ccaattatcaaggaaataat

SEQ ID NO: 577 - DNA, 20 bp, Homo sapiens
  source 1..20: mol_type = other DNA
caattatcaaggaaataatg

SEQ ID NO: 578 - DNA, 20 bp, Homo sapiens
  source 1..20: mol_type = other DNA
aattatcaaggaaataatgt

SEQ ID NO: 579 - DNA, 20 bp, Homo sapiens
  source 1..20: mol_type = other DNA
attatcaaggaaataatgtt

SEQ ID NO: 580 - DNA, 20 bp, Homo sapiens
  source 1..20: mol_type = other DNA
ttatcaaggaaataatgttt

SEQ ID NO: 581 - DNA, 20 bp, Homo sapiens
  source 1..20: mol_type = other DNA
tatcaaggaaataatgtttt

SEQ ID NO: 582 - DNA, 21 bp, Homo sapiens
  source 1..21: mol_type = other DNA
ttgctacccaatactaccctt

SEQ ID NO: 583 - DNA, 21 bp, Homo sapiens
  source 1..21: mol_type = other DNA
gtggctgccatcttcgggcct

SEQ ID NO: 584 - DNA, 21 bp, Homo sapiens
  source 1..21: mol_type = other DNA
gtgcagtccatctgcaatgct

SEQ ID NO: 585 - DNA, 21 bp, Homo sapiens
  source 1..21: mol_type = other DNA
accgatacagtggtgttaaca

SEQ ID NO: 586 - DNA, 21 bp, Homo sapiens
  source 1..21: mol_type = other DNA
aggcacctccgaaacccgatt

SEQ ID NO: 587 - DNA, 21 bp, Homo sapiens
  source 1..21: mol_type = other DNA
caggcacctccgaaacccgat

SEQ ID NO: 588 - DNA, 21 bp, Homo sapiens
  source 1..21: mol_type = other DNA
ctcctcagagagttatctaca

SEQ ID NO: 589 - DNA, 21 bp, Homo sapiens
  source 1..21: mol_type = other DNA
atggaatggttcgtgaactaa

SEQ ID NO: 590 - DNA, 21 bp, Homo sapiens
  source 1..21: mol_type = other DNA
aagccacaggccccagttatt

SEQ ID NO: 591 - DNA, 21 bp, Homo sapiens
  source 1..21: mol_type = other DNA
aaccaataaaagtgtcactaa

SEQ ID NO: 592 - DNA, 21 bp, Homo sapiens
  source 1..21: mol_type = other DNA
cccccatatcccaaataataa

SEQ ID NO: 593 - DNA, 21 bp, Homo sapiens
  source 1..21: mol_type = other DNA
aagggaaagccagcgaacatc

SEQ ID NO: 594 - DNA, 20 bp, Homo sapiens
  source 1..20: mol_type = other DNA
cgcctttctgacagtggaac

SEQ ID NO: 595 - DNA, 21 bp, Homo sapiens
  source 1..21: mol_type = other DNA
cgcctttctgacagtggaacg

SEQ ID NO: 596 - DNA, 21 bp, Homo sapiens
  source 1..21: mol_type = other DNA
ttgccaggagatggaatatca

SEQ ID NO: 597 - DNA, 21 bp, Homo sapiens
  source 1..21: mol_type = other DNA
aagcagatgttcatcacttat

SEQ ID NO: 598 - DNA, 21 bp, Homo sapiens
  source 1..21: mol_type = other DNA
atgccaattatcaaggaaata

SEQ ID NO: 599 - DNA, 20 bp, Homo sapiens
  source 1..20: mol_type = other DNA
atggtagaagaattgaggat

SEQ ID NO: 600 - DNA, 21 bp, Homo sapiens
  source 1..21: mol_type = other DNA
atggtagaagaattgaggatg

SEQ ID NO: 601 - DNA, 21 bp, Homo sapiens
  source 1..21: mol_type = other DNA
ttccttgcagtgtaatcgaca

SEQ ID NO: 602 - DNA, 21 bp, Homo sapiens
  source 1..21: mol_type = other DNA
gcggtcaccactcgacgcatc

SEQ ID NO: 603 - DNA, 21 bp, Homo sapiens
  source 1..21: mol_type = other DNA
tggctattacctatgttcgag

SEQ ID NO: 604 - DNA, 21 bp, Homo sapiens
  source 1..21: mol_type = other DNA
atcctcatttctacccgaacc

SEQ ID NO: 605 - DNA, 20 bp, Homo sapiens
  source 1..20: mol_type = other DNA
aacccaggagccgaacgcta

SEQ ID NO: 606 - DNA, 21 bp, Homo sapiens
  source 1..21: mol_type = other DNA
aacccaggagccgaacgctag

SEQ ID NO: 607 - DNA, 21 bp, Homo sapiens
  source 1..21: mol_type = other DNA
acccgaacccaggagccgaac

SEQ ID NO: 608 - DNA, 21 bp, Homo sapiens
  source 1..21: mol_type = other DNA
tgcgtgtgcaccaccaacgtt

SEQ ID NO: 609 - DNA, 21 bp, Homo sapiens
  source 1..21: mol_type = other DNA
atccagtcttcaggcgcacca

SEQ ID NO: 610 - DNA, 21 bp, Homo sapiens
  source 1..21: mol_type = other DNA
atccagtcttcaggcgcaccg

SEQ ID NO: 611 - DNA, 20 bp, Homo sapiens
  source 1..20: mol_type = other DNA
tccccacatacagacccgct

SEQ ID NO: 612 - DNA, 21 bp, Homo sapiens
  source 1..21: mol_type = other DNA
tccccacatacagacccgctg

SEQ ID NO: 613 - DNA, 20 bp, Homo sapiens
  source 1..20: mol_type = other DNA
aagcatgaatggtagtgcgt

SEQ ID NO: 614 - DNA, 21 bp, Homo sapiens
  source 1..21: mol_type = other DNA
aagcatgaatggtagtgcgtg

SEQ ID NO: 615 - DNA, 20 bp, Homo sapiens
  source 1..20: mol_type = other DNA
atggagtcaacaaccatcga

SEQ ID NO: 616 - DNA, 21 bp, Homo sapiens
  source 1..21: mol_type = other DNA
atggagtcaacaaccatcgag

SEQ ID NO: 617 - DNA, 21 bp, Homo sapiens
  source 1..21: mol_type = other DNA
ttccccacatacagacccgct

SEQ ID NO: 618 - DNA, 21 bp, Homo sapiens
  source 1..21: mol_type = other DNA
gtggtgtctgtggccgttcaa

SEQ ID NO: 619 - DNA, 21 bp, Homo sapiens
  source 1..21: mol_type = other DNA
tggcgcttcgggacccgcttt

SEQ ID NO: 620 - DNA, 21 bp, Homo sapiens
  source 1..21: mol_type = other DNA
atggtacaaacccaggcgtct

SEQ ID NO: 621 - DNA, 21 bp, Homo sapiens
  source 1..21: mol_type = other DNA
cagcactggtctcattcgttt

SEQ ID NO: 622 - DNA, 21 bp, Homo sapiens
  source 1..21: mol_type = other DNA
ttggaagtggactgcgtttat

SEQ ID NO: 623 - DNA, 21 bp, Homo sapiens
  source 1..21: mol_type = other DNA
gccgtgatcgtgtctgcggtc

SEQ ID NO: 624 - DNA, 21 bp, Homo sapiens
  source 1..21: mol_type = other DNA
tacccgaacccaggagccgaa

SEQ ID NO: 625 - DNA, 21 bp, Homo sapiens
  source 1..21: mol_type = other DNA
ggcgcttcgggacccgcttta

SEQ ID NO: 626 - DNA, 20 bp, Homo sapiens
  source 1..20: mol_type = other DNA
atcggggaagtgggtgccgt

SEQ ID NO: 627 - DNA, 21 bp, Homo sapiens
  source 1..21: mol_type = other DNA
atcggggaagtgggtgccgtg

SEQ ID NO: 628 - DNA, 21 bp, Homo sapiens
  source 1..21: mol_type = other DNA
atcgacataaaccctggcgct

SEQ ID NO: 629 - DNA, 21 bp, Homo sapiens
  source 1..21: mol_type = other DNA
atgggtttgggaagcggagac

SEQ ID NO: 630 - DNA, 21 bp, Homo sapiens
  source 1..21: mol_type = other DNA
acccaagcacaaactgtcgtc

SEQ ID NO: 631 - DNA, 21 bp, Homo sapiens
  source 1..21: mol_type = other DNA
acccctggtttccacgaggct

SEQ ID NO: 632 - DNA, 21 bp, Homo sapiens
  source 1..21: mol_type = other DNA
tgggttctccatatcgagaca

SEQ ID NO: 633 - DNA, 21 bp, Homo sapiens
  source 1..21: mol_type = other DNA
gtccctgaagtgccagcgtca

SEQ ID NO: 634 - DNA, 21 bp, Homo sapiens
  source 1..21: mol_type = other DNA
gtccctgaagtgccagcgtcg

SEQ ID NO: 635 - DNA, 21 bp, Homo sapiens
  source 1..21: mol_type = other DNA
gaccatgccgtgatcgtgtct

SEQ ID NO: 636 - DNA, 21 bp, Homo sapiens
  source 1..21: mol_type = other DNA
gccgaacgctagatcggggaa

SEQ ID NO: 637 - DNA, 21 bp, Homo sapiens
  source 1..21: mol_type = other DNA
gtggtcgatggaacgattgca

SEQ ID NO: 638 - DNA, 20 bp, Homo sapiens
  source 1..20: mol_type = other DNA
cacctccgaaacccgattca

SEQ ID NO: 639 - DNA, 21 bp, Homo sapiens
  source 1..21: mol_type = other DNA
cacctccgaaacccgattcag

SEQ ID NO: 640 - DNA, 21 bp, Homo sapiens
  source 1..21: mol_type = other DNA
tggctgccatcttcgggcctt

SEQ ID NO: 641 - DNA, 21 bp, Homo sapiens
  source 1..21: mol_type = other DNA
aaggtataatcttcgactcaa

SEQ ID NO: 642 - DNA, 21 bp, Homo sapiens
  source 1..21: mol_type = other DNA
gaggtgatgatgttactagcc

SEQ ID NO: 643 - DNA, 21 bp, Homo sapiens
  source 1..21: mol_type = other DNA
atcctctctcccctgatatct

SEQ ID NO: 644 - DNA, 21 bp, Homo sapiens
  source 1..21: mol_type = other DNA
ttcctgtggaaatatgcaacc

SEQ ID NO: 645 - DNA, 21 bp, Homo sapiens
  source 1..21: mol_type = other DNA
ctgcggtcaccactcgacgca

SEQ ID NO: 646 - DNA, 21 bp, Homo sapiens
  source 1..21: mol_type = other DNA
tcccggatgctctccgactaa

SEQ ID NO: 647 - DNA, 21 bp, Homo sapiens
  source 1..21: mol_type = other DNA
tgcggtcaccactcgacgcat

SEQ ID NO: 648 - DNA, 20 bp, Homo sapiens
  source 1..20: mol_type = other DNA
gtgctggatttctcccggat

SEQ ID NO: 649 - DNA, 21 bp, Homo sapiens
  source 1..21: mol_type = other DNA
aaaacaggcattagctatggg

SEQ ID NO: 650 - DNA, 21 bp, Homo sapiens
  source 1..21: mol_type = other DNA
tagctatgggaatgatgacaa

SEQ ID NO: 651 - DNA, 21 bp, Homo sapiens
  source 1..21: mol_type = other DNA
caggcattagctatgggaatg

SEQ ID NO: 652 - DNA, 20 bp, Homo sapiens
  source 1..20: mol_type = other DNA
aaagcaggcattagctatgg

SEQ ID NO: 653 - DNA, 21 bp, Homo sapiens
  source 1..21: mol_type = other DNA
gtgggaggcatttggtggttt

SEQ ID NO: 654 - DNA, 21 bp, Homo sapiens
  source 1..21: mol_type = other DNA
gtggtttttcacacttatcat

SEQ ID NO: 655 - DNA, 21 bp, Homo sapiens
  source 1..21: mol_type = other DNA
ttcgagagaaggtcatcgact

SEQ ID NO: 656 - DNA, 20 bp, Homo sapiens
  source 1..20: mol_type = other DNA
tggctattacctatgttcga

SEQ ID NO: 657 - DNA, 21 bp, Homo sapiens
  source 1..21: mol_type = other DNA
ccgctggaagcaccaggtgtc

SEQ ID NO: 658 - DNA, 21 bp, Homo sapiens
  source 1..21: mol_type = other DNA
atgggttctccatatcgagac

SEQ ID NO: 659 - DNA, 21 bp, Homo sapiens
  source 1..21: mol_type = other DNA
tcccatgggttctccatatca

SEQ ID NO: 660 - DNA, 21 bp, Homo sapiens
  source 1..21: mol_type = other DNA
ctcccatgggttctccatatc

SEQ ID NO: 661 - DNA, 21 bp, Homo sapiens
  source 1..21: mol_type = other DNA
cagtccatctgcaatgctctg

SEQ ID NO: 662 - DNA, 21 bp, Homo sapiens
  source 1..21: mol_type = other DNA
gtccatctgcaatgctctggg

SEQ ID NO: 663 - DNA, 20 bp, Homo sapiens
  source 1..20: mol_type = other DNA
aaccctgactcagacgtggt

SEQ ID NO: 664 - DNA, 21 bp, Homo sapiens
  source 1..21: mol_type = other DNA
aaccctgactcagacgtggtg

SEQ ID NO: 665 - DNA, 21 bp, Homo sapiens
  source 1..21: mol_type = other DNA
acccttgcaaccctgactcag

SEQ ID NO: 666 - DNA, 21 bp, Homo sapiens
  source 1..21: mol_type = other DNA
ctggtttggagttggagctct

SEQ ID NO: 667 - DNA, 21 bp, Homo sapiens
  source 1..21: mol_type = other DNA
gagctcatcaaagctccatca

SEQ ID NO: 668 - DNA, 20 bp, Homo sapiens
  source 1..20: mol_type = other DNA
ctccactggctattacctat

SEQ ID NO: 669 - DNA, 21 bp, Homo sapiens
  source 1..21: mol_type = other DNA
ctccactggctattacctatg

SEQ ID NO: 670 - DNA, 21 bp, Homo sapiens
  source 1..21: mol_type = other DNA
aagctgaccttgcagttgctc

SEQ ID NO: 671 - DNA, 21 bp, Homo sapiens
  source 1..21: mol_type = other DNA
ttgctccactggctattacct

SEQ ID NO: 672 - DNA, 21 bp, Homo sapiens
  source 1..21: mol_type = other DNA
ctggatggatttatgacgact

SEQ ID NO: 673 - DNA, 21 bp, Homo sapiens
  source 1..21: mol_type = other DNA
aaagattccttctatgtcagt

SEQ ID NO: 674 - DNA, 21 bp, Homo sapiens
  source 1..21: mol_type = other DNA
ttccttctatgtcagtctcta

SEQ ID NO: 675 - DNA, 21 bp, Homo sapiens
  source 1..21: mol_type = other DNA
ttgctggatggatttatgaca

SEQ ID NO: 676 - DNA, 21 bp, Homo sapiens
  source 1..21: mol_type = other DNA
atggatttatgacgactgata

SEQ ID NO: 677 - DNA, 21 bp, Homo sapiens
  source 1..21: mol_type = other DNA
atccttggctttacatatgaa

SEQ ID NO: 678 - DNA, 21 bp, Homo sapiens
  source 1..21: mol_type = other DNA
aaggattccttctatgtcagt

SEQ ID NO: 679 - DNA, 21 bp, Homo sapiens
  source 1..21: mol_type = other DNA
cattccattccattgtccatt

SEQ ID NO: 680 - DNA, 21 bp, Homo sapiens
  source 1..21: mol_type = other DNA
tcacgaaccattccattccat

SEQ ID NO: 681 - DNA, 21 bp, Homo sapiens
  source 1..21: mol_type = other DNA
ctcccggatgctctccgacta

SEQ ID NO: 682 - DNA, 429 bp, Homo sapiens
  source 1..429: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgc

SEQ ID NO: 683 - DNA, 469 bp, Homo sapiens
  source 1..469: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcag

SEQ ID NO: 684 - DNA, 401 bp, Homo sapiens
  source 1..401: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcg

SEQ ID NO: 685 - DNA, 477 bp, Homo sapiens
  source 1..477: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcc

SEQ ID NO: 686 - DNA, 60 bp, Homo sapiens
  source 1..60: mol_type = other DNA
gaggaggaggagagagaccgggagggcgcccgggaggcagggcgcgcgcacactccgagg

SEQ ID NO: 687 - DNA, 60 bp, Homo sapiens
  source 1..60: mol_type = other DNA
gatgctgacgaaggctcgcgaggctgtgagcagccacagtgccctgctcagaagccccgg

SEQ ID NO: 688 - DNA, 60 bp, Homo sapiens
  source 1..60: mol_type = other DNA
gtctcccgcgcccgcgcccgtgtcgccgccgtgcccgcgagcgggagccggagtcgccgc

SEQ ID NO: 689 - DNA, 60 bp, Homo sapiens
  source 1..60: mol_type = other DNA
cgtgtgcagatgcagggcgccggtgccctgcgggtgcgggtgcaggagcagcgtgtgcag

SEQ ID NO: 690 - DNA, 60 bp, Homo sapiens
  source 1..60: mol_type = other DNA
ccccacgccaccctttctggtcatctcccctcccgccccgcccctgcgcacactccctcg

SEQ ID NO: 691 - DNA, 60 bp, Homo sapiens
  source 1..60: mol_type = other DNA
tctccccggtaaagtctcgcggtgctgccgggctcagccccgtctcctcctcttgctccc

SEQ ID NO: 692 - DNA, 60 bp, Homo sapiens
  source 1..60: mol_type = other DNA
cgcctcctccgcccgccgcccgggagccgcagccgccgccgccactgccactcccgctct

SEQ ID NO: 693 - DNA, 60 bp, Homo sapiens
  source 1..60: mol_type = other DNA
tgggtgcccccacccttcccccatcctcctcccttccccactccaccctcgtcggtcccc

SEQ ID NO: 694 - DNA, 60 bp, Homo sapiens
  source 1..60: mol_type = other DNA
aaaaaaaaaaaaaaagcccaccctccagcctcgctgcaaagagaaaaccggagcagccgc

SEQ ID NO: 695 - DNA, 60 bp, Homo sapiens
  source 1..60: mol_type = other DNA
tctcgcactctcccttctcctttataaaggccggaacagctgaaagggtggcaacttctc

SEQ ID NO: 696 - DNA, 60 bp, Homo sapiens
  source 1..60: mol_type = other DNA
gccgcgtccacctgtcggccgggcccagccgagcgcgcagcgggcacgccgcgcgcgcgg

SEQ ID NO: 697 - DNA, 60 bp, Homo sapiens
  source 1..60: mol_type = other DNA
gcgccccgcccccggcgctgagtcctgtgacagcccccgggccgcctgcacttgcagcct

SEQ ID NO: 698 - DNA, 60 bp, Homo sapiens
  source 1..60: mol_type = other DNA
aaagaagagtcccctattcctgaaacttactctgtccgtggtgctgaaacattgtaccga

SEQ ID NO: 699 - DNA, 60 bp, Homo sapiens
  source 1..60: mol_type = other DNA
cgtcctcaaagagcagcaagccttctccatcttaatttgactctaccgcagagcagactt

SEQ ID NO: 700 - DNA, 60 bp, Homo sapiens
  source 1..60: mol_type = other DNA
atgcagctattctgttgtattctcattctcactctccctcccttctctcactctcactct

SEQ ID NO: 701 - DNA, 60 bp, Homo sapiens
  source 1..60: mol_type = other DNA
catgttagcgtccccagctgcagcccagggagggagagaggctgcgctcagtctgagagt

SEQ ID NO: 702 - DNA, 60 bp, Homo sapiens
  source 1..60: mol_type = other DNA
tgacgtcagagagagagtttaaaacagagggagacggttgagagcacacaagccgcttta

SEQ ID NO: 703 - DNA, 60 bp, Homo sapiens
  source 1..60: mol_type = other DNA
gagtgaaaataaaagattgtataaatcgtggggcatgtggaattgtgtgtgcctgtgcgt

SEQ ID NO: 704 - DNA, 60 bp, Homo sapiens
  source 1..60: mol_type = other DNA
gccgcggcgaggaagctccataaaagccctgtcgcgacccgctctctgcaccccatccgc

SEQ ID NO: 705 - DNA, 60 bp, Homo sapiens
  source 1..60: mol_type = other DNA
cagtcctaagtataagccctataaaatgatgggctttgaaatgctggtcagggtagagtg

SEQ ID NO: 706 - DNA, 60 bp, Homo sapiens
  source 1..60: mol_type = other DNA
ttttccattaatgttttcagactgctgttgaccacaggtaactgaaatcatggaaagaga

SEQ ID NO: 707 - DNA, 60 bp, Homo sapiens
  source 1..60: mol_type = other DNA
tggtcatatgagcagaaatgatgagaaaagcactttttaatcttttcgcacttgctctgc

SEQ ID NO: 708 - DNA, 60 bp, Homo sapiens
  source 1..60: mol_type = other DNA
aatagccagagcagaagcctatataggtggccatcccacctccaggctcacttcccgaca

SEQ ID NO: 709 - DNA, 60 bp, Homo sapiens
  source 1..60: mol_type = other DNA
cagcgctcagattttgcagcataaatttgcatccaggacagaccagagcagaggctgagg

SEQ ID NO: 710 - DNA, 60 bp, Homo sapiens
  source 1..60: mol_type = other DNA
gcacgcacgcgcgcgcagggccaagcccgaggcagctcgcccgcagctcgcactcgcagg

SEQ ID NO: 711 - DNA, 60 bp, Homo sapiens
  source 1..60: mol_type = other DNA
cccgcctctggctcgcccgaggacgcgctggcacgcctcccaccccctcactctgactcc

SEQ ID NO: 712 - DNA, 60 bp, Homo sapiens
  source 1..60: mol_type = other DNA
cactgggctccctttccctcaaatgctctggggctctccgcgctttcctgagtccgggct

SEQ ID NO: 713 - DNA, 60 bp, Homo sapiens
  source 1..60: mol_type = other DNA
cacagaaaactcctctgggccacgcttcccgcctcgccgaggtctccccagtctgcccct

SEQ ID NO: 714 - DNA, 60 bp, Homo sapiens
  source 1..60: mol_type = other DNA
ctctgcccccgcctaccccggagccgtgcagccgcctctccgaatctctctcttctcctg

SEQ ID NO: 715 - DNA, 60 bp, Homo sapiens
  source 1..60: mol_type = other DNA
ctggatttataatcgccctataaagctccagaggcggtcaggcacctgcagaggagcccc

SEQ ID NO: 716 - DNA, 61 bp, Homo sapiens
  source 1..61: mol_type = other DNA
gggacgcgcgggcggggtgggctgtgccccgcgggaaccccgccggcctgtgcgcttgctg

SEQ ID NO: 717 - DNA, 60 bp, Homo sapiens
  source 1..60: mol_type = other DNA
ctccctcccgcgctccccgcgctcgggcgccgcagagctgtccagcttcagtgccgaacc

SEQ ID NO: 718 - DNA, 60 bp, Homo sapiens
  source 1..60: mol_type = other DNA
gtactggtgtacaaggacaaggtgactttttttcttttcccagattgaaagggccaaaga

SEQ ID NO: 719 - DNA, 60 bp, Homo sapiens
  source 1..60: mol_type = other DNA
cctccgccgctcagccccggactccttacgtcagggtagcggggtcccccctccgcgcgg

SEQ ID NO: 720 - DNA, 60 bp, Homo sapiens
  source 1..60: mol_type = other DNA
ccaggagagctcggcaagtatataaggacagaggagcgcgggaccaagcggcggcgaagg

SEQ ID NO: 721 - DNA, 60 bp, Homo sapiens
  source 1..60: mol_type = other DNA
ttccttcagctgtgtcttaaagtaaatcttgttgtggagcggagccctcagctgagggag

SEQ ID NO: 722 - DNA, 60 bp, Homo sapiens
  source 1..60: mol_type = other DNA
gtaagtatcttcttcttcccctcgtgagtccctccccttttccagaatcacttgcactgt

SEQ ID NO: 723 - DNA, 60 bp, Homo sapiens
  source 1..60: mol_type = other DNA
ggggcggagcggagacagtaccttcggagataatcctttctcctgccgcagtggagagga

SEQ ID NO: 724 - DNA, 60 bp, Homo sapiens
  source 1..60: mol_type = other DNA
ccctgcctagtctccatataaaagcggcgccgcctccccgccctctctcactccccgctc

SEQ ID NO: 725 - DNA, 60 bp, Homo sapiens
  source 1..60: mol_type = other DNA
gggcggcccagccccaggttacgtcgtccccagaaagaatctggccaacagtctggccgt

SEQ ID NO: 726 - DNA, 60 bp, Homo sapiens
  source 1..60: mol_type = other DNA
atgctaatacaccttaattttacgattttttcacttttcctccccacagcgtgagtgcat

SEQ ID NO: 727 - DNA, 60 bp, Homo sapiens
  source 1..60: mol_type = other DNA
taaccccagtcccctttcttctccttccgcccctccccaaccccgccccataatggatgc

SEQ ID NO: 728 - DNA, 527 bp, Homo sapiens
  source 1..527: mol_type = other DNA
ccccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgctcgcttcggcagcacatatactaaaattggaacgatacagagaagattagcatggcccctgcgcaaggatgacacgcaaattcgtgaagcgttccatatttttacatcaggttgtttttctgtttttacatcaggttgtttttctgtttggttttttttttacaccacgtttatacgccggtgcacggtttacca

SEQ ID NO: 729 - DNA, 250 bp, Homo sapiens
  source 1..250: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccg

SEQ ID NO: 730 - DNA, 112 bp, Homo sapiens
  source 1..112: mol_type = other DNA
gagggcctatttcccatgattccttcatatttgcatatacgatagcttaccgtaacttgaaagtatttcgatttcttggctttatatatcttgtggaaaggacgaaacaccg

SEQ ID NO: 731 - DNA, 47 bp, Homo sapiens
  source 1..47: mol_type = other DNA
tttcgatttcttggctttatatatcttgtggaaaggacgaaacaccg

SEQ ID NO: 732 - DNA, 76 bp, Homo sapiens
  source 1..76: mol_type = other DNA
atacgatagcttaccgtaacttgaaagtatttcgatttcttggctttatatatcttgtggaaaggacgaaacaccg

SEQ ID NO: 733 - DNA, 80 bp, Homo sapiens
  source 1..80: mol_type = other DNA
gagggcctatttcccatgattccttcatatttgcattttcgatttcttggctttatatatcttgtggaaaggacgaaacg

SEQ ID NO: 734 - DNA, 100 bp, Homo sapiens
  source 1..100: mol_type = other DNA
aatatttgcatgtcgctatgtgttctgggaaatcaccataaacgtgaaatgtctttggatttgggaatcttataagttctgtatgagaccactctttccc

SEQ ID NO: 735 - DNA, 244 bp, Homo sapiens
  source 1..244: mol_type = other DNA
ctgcagtatttagcatgccccacccatctgcaagtgcattctggatagtgtcaaaacaggcggaaatcaagtccgtttatctcaaactttagcattttgggaataaatgatatttgctatgctggttaaattagattttagttaaatttcctgatgaagctctagtacgataagcaacttgacctaagtgtaaagttgagatttccttcaggtttatatagcttgtgcgccgcctgggtacctc

SEQ ID NO: 736 - DNA, 723 bp, synthetic construct
  source 1..723: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 737 - DNA, 1725 bp, synthetic construct
  source 1..1725: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 738 - DNA, 594 bp, Gallus gallus
  source 1..594: mol_type = other DNA
cgcgtggtacctctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcgg

SEQ ID NO: 739 - DNA, 865 bp, synthetic construct
  source 1..865: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 740 - DNA, 251 bp, Mus musculus
  source 1..251: mol_type = other DNA
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 741 - DNA, 212 bp, Homo sapiens
  source 1..212: mol_type = other DNA
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 742 - DNA, 460 bp, Homo sapiens
  source 1..460: mol_type = other DNA
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 743 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 744 - DNA, 82 bp, synthetic construct
  source 1..82: mol_type = other DNA
gtgagtatctcagggatccagacatggggatatgggaggtgcctctgatcccagggctcactgtgggtctctctgttcacag

SEQ ID NO: 745 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 746 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 747 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 748 - DNA, 143 bp, synthetic construct
  source 1..143: mol_type = other DNA
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 749 - DNA, 110 bp, synthetic construct
  source 1..110: mol_type = other DNA
ttggccactccctctctgcgcgctcgctcgctcactgaggccgggcgaccaaaggtcgcccgacgcccgggctttgcccgggcggcctcagtgagcgagcgagcgcgcag

SEQ ID NO: 750 - DNA, 55 bp, Oryctolagus cuniculus
  source 1..55: mol_type = other DNA
ataaaggaaatttattttcattgcaatagtgtgttggaattttttgtgtctctca

SEQ ID NO: 751 - DNA, 127 bp, Oryctolagus cuniculus
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 752 - DNA, 128 bp, Homo sapiens
  source 1..128: mol_type = other DNA
gtttgaatgaggcttcagtactttacagaatcgttgcctgcacatcttggaaacacttgctgggattacttcttcaggttaacccaacagaaggctcgagaaggtatattgctgttgacagtgagcgc

SEQ ID NO: 753 - DNA, 122 bp, Homo sapiens
  source 1..122: mol_type = other DNA
ttgcctactgcctcggaattcaaggggctactttaggagcaattatcttgtttactaaaactgaataccttgctatctctttgatacatttttacaaagctgaattaaaatggtataaatta

SEQ ID NO: 754 - DNA, 129 bp, Homo sapiens
  source 1..129: mol_type = other DNA
gtttgaatgaggcttcagtactttacagaatcgttgcctgcacatcttggaaacacttgctgggattacttcttcaggttaacccaacagaaggctcgagaaggtatattgctgttgacagtgagcgac

SEQ ID NO: 755 - DNA, 123 bp, Homo sapiens
  source 1..123: mol_type = other DNA
gctgcctactgcctcggaattcaaggggctactttaggagcaattatcttgtttactaaaactgaataccttgctatctctttgatacatttttacaaagctgaattaaaatggtataaatta

SEQ ID NO: 756 - DNA, 128 bp, Homo sapiens
  source 1..128: mol_type = other DNA
gtttgaatgaggcttcagtactttacagaatcgttgcctgcacatcttggaaacacttgctgggattacttcttcaggttaacccaacagaaggctcgagaaggtatattgctgttgacagtgagcga

SEQ ID NO: 757 - DNA, 121 bp, Homo sapiens
  source 1..121: mol_type = other DNA
tgcctactgcctcggaattcaaggggctactttaggagcaattatcttgtttactaaaactgaataccttgctatctctttgatacatttttacaaagctgaattaaaatggtataaatta

SEQ ID NO: 758 - DNA, 17 bp, Homo sapiens
  source 1..17: mol_type = other DNA
tagtgaagccacagatg

SEQ ID NO: 759 - DNA, 129 bp, Homo sapiens
  source 1..129: mol_type = other DNA
gtttgaatgaggcttcagtactttacagaatcgttgcctgcacatcttggaaacacttgctgggattacttcttcaggttaacccaacagaaggctaaagaaggtatattgctgttgacagtgagcgac

SEQ ID NO: 760 - DNA, 123 bp, Homo sapiens
  source 1..123: mol_type = other DNA
gctgcctactgcctcggacttcaaggggctactttaggagcaattatcttgtttactaaaactgaataccttgctatctctttgatacatttttacaaagctgaattaaaatggtataaatta

SEQ ID NO: 761 - DNA, 19 bp, Homo sapiens
  source 1..19: mol_type = other DNA
ctgtgaagccacagatggg

SEQ ID NO: 762 - DNA, 84 bp, Homo sapiens
  source 1..84: mol_type = other DNA
caaaccaggaaggggaaatctgtggtttaaattctttatgcctcatcctctgagtgctgaaggcttgctgtaggctgtatgctg

SEQ ID NO: 763 - DNA, 203 bp, Homo sapiens
  source 1..203: mol_type = other DNA
cagtgtatgatgcctgttactagcattcacatggaacaaattgctgccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagta

SEQ ID NO: 764 - DNA, 13 bp, Homo sapiens
  source 1..13: mol_type = other DNA
tttgcctccaact

SEQ ID NO: 765 - DNA, 106 bp, Homo sapiens
  source 1..106: mol_type = other DNA
acgtttccagaacgtctgtagcttttctcctccttccctccattttcctcttggtcttacctttggcctagtggttggtgtagtgataatgtagcgagattttctg

SEQ ID NO: 766 - DNA, 124 bp, Homo sapiens
  source 1..124: mol_type = other DNA
tggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 767 - DNA, 20 bp, Homo sapiens
  source 1..20: mol_type = other DNA
ggttgcgaggtatgagtaaa

SEQ ID NO: 768 - DNA, 212 bp, Homo sapiens
  source 1..212: mol_type = other DNA
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctc

SEQ ID NO: 769 - DNA, 213 bp, Homo sapiens
  source 1..213: mol_type = other DNA
gagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 770 - DNA, 18 bp, Homo sapiens
  source 1..18: mol_type = other DNA
atttaatgtctatacaat

SEQ ID NO: 771 - DNA, 17 bp, synthetic construct
  source 1..17: mol_type = other DNA
tttgtgagggtctggtc

SEQ ID NO: 772 - DNA, 19 bp, synthetic construct
  source 1..19: mol_type = other DNA
aaarcaggcattagctatg

SEQ ID NO: 773 - DNA, 19 bp, Homo sapiens
  source 1..19: mol_type = other DNA
aaaacaggcattagctatg

SEQ ID NO: 774 - DNA, 19 bp, Mus musculus
  source 1..19: mol_type = other DNA
aaagcaggcattagctatg

SEQ ID NO: 775 - DNA, 1541 bp, synthetic construct
  source 1..1541: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 776 - skipped

SEQ ID NO: 777 - DNA, 2893 bp, synthetic construct
  source 1..2893: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttcgatcctatcacgagactagcctcgagaagcttgatatcagcacccacatagcagctcacaaatgtctgaaactccaattcttgggaatctgacacgatcacacatgcaggcaaaataccaatgtacatgaattaaaaaaaaaaaaaacaacctttaaaagaaacaagggttcagtaccactactgacatcttgtttccccagaggccttactttaattatttattgtttccacttagttgctcaattaattaatttagaggtttttttcttcctttctttttcttttttctttctctcttttttttcttcttaagacagggtttctctgtgtagctcaggctatcctggaactcactctgtagaccaggctggccttgtactcaaagatctgcctgcctctgcctccccagtgctgggattaaagacatgcaccatcactgccctgctttcctctttttattttgaaaattgttcatcaacagttactaaacgtgttcgaattccaagagctgactagacatataagaccattcagccttctgaataagatgtaggtgtgcccctcctcttactcctctatttggaagttggttactttctgtatgtagtatgcgaatccccctctgccaccccgctttctgttttaaaacagaaaaggctgcaacatacagtgtgtgcttctgttcttgaactggaagcttaggctgtcctggacttgggttgagacctgggctcatccagataggaaatggatttggtgaccccgccaggacttcgcaggcaccacatcgtggtcgtgtgtgggtgctgtatgcacccactgattgcgcgcgtgggttccagagcttggtggtctgcgagaggagagtgggcaagagtgggtgtgtctgtggagccccagctaggggctgctgcccgctgctcccacttgtggctcctgggcgccgccagcaggcacatctccggaggacgccgcgggatgggagctgatgacaggagagcgccgtctcccgagtgatggcagcgcacgctgctgcctcgccgcctccgccgctcagtcctgatcttacgttagggtagctgggtaccccctccgcccgggaaccagctagtagagggagaacagagcagagcgtgcggcagagccgatcccgcgtcccgccgaaccctgccaagccccgccaatcccagcagagcaggaaccagcgcagctgagccaacaccggacgccgcactgagacccagcattccccagccgccactacccggtccccgccggggtgccgggctcgtcctgtgagcccctcgtcatgcgtgtcgggctcttcgactctccagatcagttccagagcgctgagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 778 - DNA, 311 bp, synthetic construct
  source 1..311: mol_type = other DNA
gtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaatta

SEQ ID NO: 779 - DNA, 1289 bp, synthetic construct
  source 1..1289: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggtaccctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcacgggatccaagcttgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggagagatcgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 780 - DNA, 311 bp, synthetic construct; identical to SEQ ID NO: 778
  source 1..311: mol_type = other DNA

SEQ ID NO: 781 - DNA, 1290 bp, synthetic construct
  source 1..1290: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggtacccggccgccgagtgagagacacaaaaaattccaacacactattgcaatgaaaataaatttcctttattagccagaagtcagatgctcaaggggcttcatgatgtccccataatttttggcagagggaaaaagatcggatcccgtgataatttataccattttaattcagctttgtaaaaatgtatcaaagagatagcaaggtattcagttttagtaaacaagataattgctcctaaagtagccccttgaattccgaggcagtaggcaataaaacaggcattagctatgggcatctgtggcttcactacccatagctaatgcctgttttagcgctcactgtcaacagcaatataccttctcgagccttctgttgggttaacctgaagaagtaatcccagcaagtgtttccaagatgtgcaggcaacgattctgtaaagtactgaagcctcattcaaacaattaccctgttatccctagtcgagtcgacctagcccgggctaggcgcgccctgcgctctcaggcacgacacgactcctccgctgcaattggcagactgaggcagcgctgagtcgccggcgccgcagcgcagatggtcgcgcccgtgcccccctatctcgcgcctcgcgtggtgcggtccggctgggccggcggcggcgcggacgcgaccaaggtggccgggaaggggagtttgcgggggaccggcgagtgacgtcagcgcgccttcagtgctgaggcggcggtggcgcgcgccgccaggcgggggcgaaggcactgtccgcggtgctgaagctggcagtgcgcacgcgcctcgccgcatcctgtttcccctccccctctctgataggggatgcgcaatttggggaatgggggttgggtgcttgtccagtgggtcggggtcggtcgtcaggtaggcacccccaccccgcctcatcctggtcctaaaacccacttgcactcatacgcagggccctctgcaggctagcgaagcaattctagcaggcatgctggggagagatcgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 782 - DNA, 311 bp, synthetic construct
  source 1..311: mol_type = other DNA
taatttataccattttaattcagctttgtaaaaatgtatcaaagagatagcaaggtattcagttttagtaaacaagataattgctcctaaagtagccccttgaattccgaggcagtaggcaataaaacaggcattagctatgggcatctgtggcttcactacccatagctaatgcctgttttagcgctcactgtcaacagcaatataccttctcgagccttctgttgggttaacctgaagaagtaatcccagcaagtgtttccaagatgtgcaggcaacgattctgtaaagtactgaagcctcattcaaac

SEQ ID NO: 783 - DNA, 2247 bp, synthetic construct
  source 1..2247: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcacgggatccggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcacgggatccggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 784 - DNA, 2247 bp, synthetic construct
  source 1..2247: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcacgggatccggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgacatgggttctccatatcgagactagtgaagccacagatggtctcgatatggagaacccatgctgcctactgcctcggaattcaaggggctactttaggagcaattatcttgtttactaaaactgaataccttgctatctctttgatacatttttacaaagctgaattaaaatggtataaattatcacgggatccggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgaaatccttggctttacatatgaatagtgaagccacagatgttcatatgtaaagccaaggattctgcctactgcctcggaattcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 785 - DNA, 3643 bp, synthetic construct
  source 1..3643: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatgggatttaatgtctatacaatcccatagctaatgcctgttttagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcggccgcccgagtttaattggtttatagaactcttcaagctagcactagtgaagcaattcgttgcattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgaattccaattggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacaccggtctcgagggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 786 - DNA, 3594 bp, synthetic construct
  source 1..3594: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatgggatttaatgtctatacaatcccatagctaatgcctgttttagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcggccgcccgagtttaattggtttatagaactcttcaagctagcactagtgaagcaattcgttgcattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgaattccaattggcgcgcctagcccgggctaggtcgacacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgaccggtctcgagggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 787 - DNA, 3649 bp, synthetic construct
  source 1..3649: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcggccgcccgagtttaattggtttatagaactcttcaagctagcactagtgaagcaattcgttgcattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgaattccaattggcgcgcctagcccgggctaggtcgactctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatgggatttaatgtctatacaatcccatagctaatgcctgttttagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtaccggtctcgagggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 788 - DNA, 3649 bp, synthetic construct
  source 1..3649: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcggccgcccgagtttaattggtttatagaactcttcaagctagcactagtgaagcaattcgttgcattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgaattccaattggcgcgcctagcccgggctaggtcgactctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtaccggtctcgagggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 789 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 790 - DNA, 477 bp, synthetic construct
  source 1..477: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcc

SEQ ID NO: 791 - DNA, 1357 bp, synthetic construct
  source 1..1357: mol_type = other DNA
cgatcctatcacgagactagcctcgagaagcttgatatcagcacccacatagcagctcacaaatgtctgaaactccaattcttgggaatctgacacgatcacacatgcaggcaaaataccaatgtacatgaattaaaaaaaaaaaaaacaacctttaaaagaaacaagggttcagtaccactactgacatcttgtttccccagaggccttactttaattatttattgtttccacttagttgctcaattaattaatttagaggtttttttcttcctttctttttcttttttctttctctcttttttttcttcttaagacagggtttctctgtgtagctcaggctatcctggaactcactctgtagaccaggctggccttgtactcaaagatctgcctgcctctgcctccccagtgctgggattaaagacatgcaccatcactgccctgctttcctctttttattttgaaaattgttcatcaacagttactaaacgtgttcgaattccaagagctgactagacatataagaccattcagccttctgaataagatgtaggtgtgcccctcctcttactcctctatttggaagttggttactttctgtatgtagtatgcgaatccccctctgccaccccgctttctgttttaaaacagaaaaggctgcaacatacagtgtgtgcttctgttcttgaactggaagcttaggctgtcctggacttgggttgagacctgggctcatccagataggaaatggatttggtgaccccgccaggacttcgcaggcaccacatcgtggtcgtgtgtgggtgctgtatgcacccactgattgcgcgcgtgggttccagagcttggtggtctgcgagaggagagtgggcaagagtgggtgtgtctgtggagccccagctaggggctgctgcccgctgctcccacttgtggctcctgggcgccgccagcaggcacatctccggaggacgccgcgggatgggagctgatgacaggagagcgccgtctcccgagtgatggcagcgcacgctgctgcctcgccgcctccgccgctcagtcctgatcttacgttagggtagctgggtaccccctccgcccgggaaccagctagtagagggagaacagagcagagcgtgcggcagagccgatcccgcgtcccgccgaaccctgccaagccccgccaatcccagcagagcaggaaccagcgcagctgagccaacaccggacgccgcactgagacccagcattccccagccgccactacccggtccccgccggggtgccgggctcgtcctgtgagcccctcgtcatgcgtgtcgggctcttcgactctccagatcagttccagagcgct

SEQ ID NO: 792 - DNA, 132 bp, synthetic construct
  source 1..132: mol_type = other DNA
gatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 793 - DNA, 19 bp, synthetic construct
  source 1..19: mol_type = other DNA
tagcaggcatgctggggag

SEQ ID NO: 794 - DNA, 339 bp, synthetic construct
  source 1..339: mol_type = other DNA
tcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcac

SEQ ID NO: 795 - DNA, 311 bp, synthetic construct; identical to SEQ ID NO: 778
  source 1..311: mol_type = other DNA

SEQ ID NO: 796 - DNA, 1541 bp, synthetic construct
  source 1..1541: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgacatgggttctccatatcgagactagtgaagccacagatggtctcgatatggagaacccatgctgcctactgcctcggaattcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 797 - DNA, 339 bp, synthetic construct
  source 1..339: mol_type = other DNA
tcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgacatgggttctccatatcgagactagtgaagccacagatggtctcgatatggagaacccatgctgcctactgcctcggaattcaaggggctactttaggagcaattatcttgtttactaaaactgaataccttgctatctctttgatacatttttacaaagctgaattaaaatggtataaattatcac

SEQ ID NO: 798 - DNA, 341 bp, synthetic construct
  source 1..341: mol_type = other DNA
tcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcac

SEQ ID NO: 799 - DNA, 292 bp, synthetic construct
  source 1..292: mol_type = other DNA
acgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 800 - DNA, 487 bp, synthetic construct
  source 1..487: mol_type = other DNA
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 801 - DNA, 487 bp, synthetic construct
  source 1..487: mol_type = other DNA
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatgggatttaatgtctatacaatcccatagctaatgcctgttttagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 802 - DNA, 1289 bp, synthetic construct
  source 1..1289: mol_type = other DNA
cattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 803 - DNA, 2619 bp, synthetic construct
  source 1..2619: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccgcccgagtttaattggtttatagaactcttca

SEQ ID NO: 804 - DNA, 3153 bp, synthetic construct
  source 1..3153: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 805 - DNA, 2564 bp, synthetic construct
  source 1..2564: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattcacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccgcccgagtttaattggtttatagaactcttca

SEQ ID NO: 806 - DNA, 3098 bp, synthetic construct
  source 1..3098: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattcacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 807 - DNA, 2759 bp, synthetic construct
  source 1..2759: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccgcccgagtttaattggtttatagaactcttca

SEQ ID NO: 808 - DNA, 3293 bp, synthetic construct
  source 1..3293: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 809 - DNA, 2759 bp, synthetic construct
  source 1..2759: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatgggatttaatgtctatacaatcccatagctaatgcctgttttagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccgcccgagtttaattggtttatagaactcttca

SEQ ID NO: 810 - DNA, 3293 bp, synthetic construct
  source 1..3293: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatgggatttaatgtctatacaatcccatagctaatgcctgttttagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 811 - DNA, 2892 bp, synthetic construct
  source 1..2892: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttga

SEQ ID NO: 812 - DNA, 3432 bp, synthetic construct
  source 1..3432: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 813 - DNA, 2920 bp, synthetic construct
  source 1..2920: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcggccgcccgagtttaattggtttatagaactcttcaagctagcactagtgaagcaattcgttgcattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgaattccaattggcgcgcctagcccgggctaggtcgacacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgaccggtctcgagggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttca

SEQ ID NO: 814 - DNA, 3454 bp, synthetic construct
  source 1..3454: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcggccgcccgagtttaattggtttatagaactcttcaagctagcactagtgaagcaattcgttgcattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgaattccaattggcgcgcctagcccgggctaggtcgacacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgaccggtctcgagggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 815 - DNA, 1602 bp, synthetic construct
  source 1..1602: mol_type = other DNA
atagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttga

SEQ ID NO: 816 - DNA, 29 bp, synthetic construct
  source 1..29: mol_type = other DNA
cgagtttaattggtttatagaactcttca

SEQ ID NO: 817 - DNA, 971 bp, synthetic construct
  source 1..971: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 818 - DNA, 1543 bp, synthetic construct
  source 1..1543: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 819 - DNA, 916 bp, synthetic construct
  source 1..916: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattcacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 820 - DNA, 1488 bp, synthetic construct
  source 1..1488: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattcacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 821 - DNA, 1111 bp, synthetic construct
  source 1..1111: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 822 - DNA, 1683 bp, synthetic construct
  source 1..1683: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 823 - DNA, 1602 bp, synthetic construct; identical to SEQ ID NO: 815
  source 1..1602: mol_type = other DNA

SEQ ID NO: 824 - DNA, 837 bp, synthetic construct
  source 1..837: mol_type = other DNA
gtgagcggcggatgcccttctcctctggctgtaattagtcttggtttacttgtttcttttctgtggctgttgaaagccttgaggggctctggagggccctttgtgtgggggagtgcttgggggtgttgttgtgtgtgtgttggggagtctttgtgctcttctgcctgtgctgtgagtctgtggttgttgggctttgtgtctctcagtgtgttaggggagttgctggggtgtgcccttgtgtgggggggctgtaggggaacaaaggctgttgtgggtgtgtgttgggggggtgagcagggggtgtgggttttgttggctgcaaccccccctgcacccccctccctagttgctgagcatgcctgctttggtgtgggctcttatgggttggttgggcttcttgctggtgggggtggtgcaggtgggggtgctggtgggtgggctccttggctgggagggcttggggaggggttgtgcccctgagtctgtgctgttaggttgtagctcagccattgccttttttgtagagggtcagggacttcctttgtcccaaatctgtgtgagctaaatctgggaggtctctcaccccctctagtggttgggtaagtgtgtgtctgcaggaaggaaagggtgggagggccttttgtttcttctcttccccttctccctctccagccttgggctgtcttgggggatgctgcctttggggggatgggcagggtgggtttgcttctggttgtgactgtgctctagagcctctgctaacctgttctgccttcttctttttcctacagctcctgggcaattgctggttattgtgctgtctctattttggcaaag